(19) United States
(12) Patent Application Publication
(10) Pub. No.: US 2026/0098025 A1
(43) Pub. Date: Apr. 9, 2026

(54) DIMERIC COMPOUNDS AS INHIBITORS OF GLYCOGEN SYNTHASE 1 (GYS1) AND METHODS OF USE THEREOF

(71) Applicant: Maze Therapeutics, Inc., South San Francisco, CA (US)

(72) Inventors: David John MORGANS, JR., Los Altos, CA (US); Kevin MELLEM, Redwood City, CA (US); Patrick Sang Tae LEE, Walnut Creek, CA (US); Christopher Joseph SINZ, Walnut Creek, CA (US); Alexander Wayne SCHAMMEL, San Francisco, CA (US); Chris ZIEBENHAUS, San Francisco, CA (US); Jessica WAHLERS, South San Francisco, CA (US); Christos TZITZILONIS, Emeryville, CA (US); Nathan FASTMAN, Palo Alto, CA (US)

(21) Appl. No.: 19/111,739

(22) PCT Filed: Sep. 13, 2023
(86) PCT No.: PCT/US2023/074115

§ 371 Date: Mar. 13, 2025

Related U.S. Application Data
(60) Provisional application No. 63/406,683, filed on Sep. 14, 2022.

(51) Int. Cl. C07D 401/14; A61K 31/444; A61K 31/513; A61K 38/47; A61K 47/55; C07D 213/61

(52) U.S. Cl. CPC C07D 401/14; A61K 31/444; A61K 31/513; A61K 38/47; A61K 47/55; C07D 213/61; C12Y 302/0102

(57) ABSTRACT

Provided herein are compounds of formula (I): (G1-Z1)-L-(G2-Z2), or a stereoisomer or tautomer thereof, or a pharmaceutically acceptable salt of any of the foregoing, wherein G1, G2, Z1, Z2, and L, are as defined elsewhere herein. Also provided herein are methods of preparing compounds of formula (I). Also provided herein are methods of inhibiting GYS1 and methods of treating a GYS1-mediated disease, disorder, or condition in an individual in need thereof.

### CROSS REFERENCE TO RELATED APPLICATIONS

This application claims priority to U.S. Provisional Application No. 63/406,683, filed on Sep. 14, 2022, the entire content of which is incorporated herein by reference for all purposes.

### BACKGROUND OF THE INVENTION

Pathological accumulation of glycogen is a hallmark of several devastating and chronic human diseases. For some of these disorders, the cellular etiology driving this aberrant accumulation has clear genetic underpinnings and for others the mechanistic driving force is more complex. Nonetheless, the consequence of elevated levels of glycogen is altered cellular homeostasis and impaired tissue function over time. The rate limiting enzyme in the glycogen synthesis pathway is the protein Glycogen Synthase (GYS). In humans there are two isoforms GYS1 & GYS2. The former is ubiquitously expressed but highly abundant in muscle cells, while the latter is expressed exclusively in liver. Glycogen synthesis ultimately begins with transport of glucose into cells via the GLUT transporter family of proteins. Conversion of glucose into glycogen follows along a well characterized biochemical conversion pathway to the step where GYS covalently links glucose molecules into long branches via α1,4-glycosidic linkages. The final spherical structure of glycogen results from the action of Glycogen Branching Enzyme (GBE) which introduces α1,6-linkage branch points along the strands. The result of this biochemical chain of events is the generation of an energy dense and highly soluble molecule that can be stored in the cytosol of cells and rapidly catabolized into glucose for energy usage when needed. An imbalance in the equilibrium of either glycogen synthesis or glycogenolysis can result in aberrant accumulation of cellular stores of glycogen. It has long been hypothesized that substrate reduction therapy targeted to inhibit glycogen synthase could be an effective treatment for diseases of glycogen storage. Indeed, substrate reduction therapy drugs have been very successful in modulating patient disease course in other storage disorders including Gaucher and Fabry diseases (Platt F M, Butters T D. Substrate Reduction Therapy. Lysosomal Storage Disorders, Springer US chapter 11, pgs 153-168, 2007.; Shemesh E, et al. Enzyme replacement and substrate reduction therapy for Gaucher disease. Cochrane Database of Systematic Reviews, Issue 3, 2015). It is the aim of this invention to inhibit glycogen synthase enzyme activity resulting in reduction of tissue glycogen stores with therapeutic benefit to patients suffering the consequences of aberrant cellular glycogen accumulation.

GYS1 exists as a homotetramer in complex with glycogenin (Fastman N M, et al. The structural mechanism of human glycogen synthesis by the GYS1-GYG1 complex. Cell Reports, vol. 40, 2022; McCorvie, T J, et al. Molecular basis for the regulation of human glycogen synthase by phosphorylation and glucose-6-phosphate. Nature Structural & Molecular Biology, vol. 29, 2022).

Pompe Disease is a rare genetic disorder caused by the pathological buildup of cellular glycogen due to loss of function (LOF) mutations in the lysosomal enzyme α-glucosidase (GAA). GAA catabolizes lysosomal glycogen and in its absence, glycogen builds up in lysosomes. This triggers a disease cascade beginning with lysosome and autophagosome dysfunction, leading ultimately to cell death and muscle atrophy over time (Young S P, et al. Genetics in Medicine, vol 11, no. 11, 2009). In humans, the clinical manifestation of the disease results in a spectrum of severity and occurs at a prevalence of one in 40,000 live births (Meena N K, Raben N. Pompe disease: new developments in an old lysosomal storage disorder. Biomolecules, vol. 10, 2020). Infantile onset patients are born with cellular pathology and rapidly develop severe impairments including myopathy, heart defects, organomegaly, and hypotonia which collectively left untreated will take the child's life within a year. The later onset children may develop heart enlargement but are characterized consistently by the progressive loss of motor function, degeneration of skeletal muscle, and ultimate failure of the respiratory system leading to early death. Late onset adult Pompe patients exhibit normal heart function but develop progressive muscle weakness and respiratory decline then failure. The current standard of care for Pompe patients is enzyme replacement therapy (ERT) with recombinant human GAA. ERT treatment has been successful in slowing the rate of disease progression but in the majority of patients there remains incredible unmet need (Schoser B, et al. The humanistic burden of Pompe disease: are there still unmet needs? A systematic review. BMC Neurology, vol. 17, 2017). For over a decade, substrate reduction therapy targeting GYS1 has been hypothesized to beneficial for the treatment of Pompe disease. In fact, three separate preclinical modalities have demonstrated that GYS1 genetic LOF in Pompe model mice effectively reduces tissue glycogen and improves mouse disease outcomes (Douillard-Guilloux G, et al. Modulation of glycogen synthesis by RNA interference: towards a new therapeutic approach for glycogenosis type II. Human Molecular Genetics, vol. 17, no. 24, 2008; Douillard-Guilloux G, et al. Restoration of muscle functionality by genetic suppression of glycogen synthesis in a murine model of Pompe disease. Human Molecular Genetics, vol. 19, no. 4, 2010; Clayton N P, et al. Antisense oligonucleotide-mediated suppression of muscle glycogen synthase 1 synthesis as an approach for substrate reduction therapy of Pompe Disease. Molecular Therapy—Nucleic Acids, vol. 3, 2014). A small molecule GYS1 inhibitor could be used to address the current unmet needs for Pompe patients either as a single therapy or in combination with standard of care ERT.

Pompe disease is only one of more than a dozen diseases caused by an inborn error of metabolism that result in aberrant build-up of glycogen in various tissues of the body. For some glycogen storage diseases (GSDs), specific dietary regimes effectively manage the disease but for others there are no clinically approved therapeutic interventions to modify disease course. Therefore, inhibition of glycogen synthesis and the concomitant reduction in tissue glycogen levels may be a viable treatment option for these patients. Cori disease, GSD III, is caused by mutations in the glycogen debranching enzyme (GDE) which results in pathological glycogen accumulation in the heart, skeletal muscle, and liver (Kishnani P, et al. Glycogen storage disease type III diagnosis and management guidelines. Genetics in Medicine, vol. 12, no. 7, 2010). While dietary management can be effective in ameliorating aspects of the disease there is currently no treatment to prevent the progressive myopathy in GSD III. Adult polyglucosan body disease (APBD) is an adult-onset disorder caused by loss of activity in the glycogen branching enzyme (GBE1). Deficiency in GBE results in accumulation of long strands of unbranched glycogen which precipitate in the cytosol generating polyglucosan bodies, and ultimately triggering neurological deficits in both the central and peripheral nervous systems. Genetic deletion of GYS1 in the APBD mouse model rescued deleterious accumulation of glycogen, improved life span, and neuromuscular function (Chown E E, et al. GYS1 or PPP1R3C deficiency rescues murine adult polyglucosan body disease. Annals of Clinical and Translational Neurology, vol. 7, no. 11, 2020). Lafora Disease (LD) is a very debilitating juvenile onset epilepsy disorder also characterized by accumulation of polyglucosan bodies. Genetic cross of LD mouse models with GYS1 knock out (KO) mice resulted in rescue of disease phenotypes (Pedersen B, et al. Inhibiting glycogen synthesis prevents Lafora disease in a mouse model. Annals of Neurology, vol. 74, no. 2, 2013; Varea O, et al. Suppression of glycogen synthesis as a treatment for Lafora disease: establishing the window of opportunity. Neurobiology of Disease, 2020).

The reliance on high levels of glycogen by clear cell cancers has recently emerged as a novel therapeutic target. Ewing sarcoma (ES), clear cell renal cell carcinoma (ccRCC), glycogen rich clear cell carcinoma breast cancer (GRCC), acute myeloid leukemia (AML), and nonsmall-cell lung carcinoma (NSCLC) are all examples of cancers histopathologically defined by PAS+ abnormally high levels of cellular glycogen. Elevated transcriptional levels of GYS1 have been significantly correlated with poor disease outcomes in NSCLC (Giatromanolaki A, et al. Expression of enzymes related to glucose metabolism in non-small cell lung cancer and prognosis. Experimental Lung Research, vol. 43, no. 4-5, 2017) and AML (Falantes J F, et al. Overexpression of GYS1, MIF, and MYC is associated with adverse outcome and poor response to azacitidine in myelodysplastic syndromes and acute myeloid leukemia. Clinical Lymphoma, Myeloma & Leukemia, vol. 15, no. 4, 2015). Lentiviral knockdown of GYS1 in cultured myeloid leukemia cells potently inhibited in vitro cancer cell growth and in vivo tumorigenesis (Bhanot H, et al. Pathological glycogenesis through glycogen synthase I and suppression of excessive AMP kinase activity in myeloid leukemia cells. Leukemia, vol. 29, no. 7, 2015). Genetic knock-down of GYS1 in ccRCC cell models both suppresses tumor growth in vivo and increases the synthetic lethality of sunitinib (Chen S, et al. GYS1 induces glycogen accumulation and promotes tumor progression via the NF-kB pathway in clear cell renal carcinoma. Theranostics, vol. 10, no. 20, 2020).

Reduction of GYS1 enzyme activity and reduced cellular stores of glycogen in preclinical models of Pompe disease, APBD, LD, AML, ccRCC, and NSCLC all provide compelling evidence of the potential therapeutic benefit of inhibiting glycogen synthesis. It is the aim of this invention to inhibit glycogen synthase enzyme activity resulting in reduction of tissue glycogen stores with therapeutic benefit to patients suffering the consequences of accumulated cellular glycogen.

### BRIEF SUMMARY OF THE INVENTION

In one aspect, provided herein is a compound of formula (I):

(G1-Z1)-L-(G2-Z2) (I),

or a stereoisomer or tautomer thereof, or a pharmaceutically acceptable salt of any of the foregoing, wherein:
(i) G1 and/or G1-Z1, and (ii) G2 and/or G2-Z2 are each, independently, a GYS1 inhibiting moiety; and
L is a linker.

In one aspect, provided herein is a compound of formula (I), or a stereoisomer or tautomer thereof, or a pharmaceutically acceptable salt of any of the foregoing, wherein (G1-Z1) and (G2-Z2) are each, independently, of formula (I-1) or (I-2):

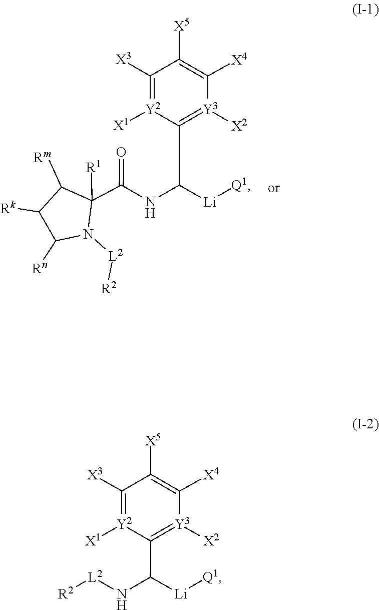

or a stereoisomer or tautomer thereof, or a pharmaceutically acceptable salt of any of the foregoing, wherein:
Y² and Y³ are each C, or
one of Y² and Y³ is N and the other of Y² and Y³ is C;
X¹ and X² are each independently H, C₁₋₆alkyl, or C₁₋₆alkoxy;
X³ and X⁴ are each independently H, halo, C₁₋₆alkyl, C₁₋₆alkoxy, or 5-20 membered heteroaryl, wherein the C₁₋₆alkyl of X³ and X⁴ is optionally substituted with one of more halo;
X⁵ is H, C₁₋₆alkyl, C₁₋₆alkoxy, or C₃₋₁₀cycloalkyl;
either
(1) L¹ is absent; and
Q¹ is selected from (i) to (iv):
(i) phenyl, wherein the phenyl of Q¹ is substituted with one or more halo, C₁₋₆alkyl, C₂₋₆alkenyl, —NH₂, —NH—C(O)—(C₁₋₆alkyl), —NH—C(O)-(3-15 membered heterocyclyl), C₃₋₁₀cycloalkyl, or 5-20 membered heteroaryl, wherein
   the C₁₋₆alkyl is optionally substituted with one or more halo, —NH—C(O)—NH(C₁₋₆alkyl), —NH—C(O)—C₁₋₆alkyl, or —NH—C(O)—C₁₋₆alkoxy,
   the C₃₋₁₀cycloalkyl is optionally substituted with one or more halo or C₁₋₆alkyl, and
   the 5-20 membered heteroaryl is optionally substituted with one or more C₁₋₆alkyl,
(ii) 3-15 membered heterocyclyl, wherein the 3-15 membered heterocyclyl of Q¹ is optionally substituted with one or more oxo, or C₁₋₆alkyl,
(iii) 5-20 membered heteroaryl, wherein the 5-20 membered heteroaryl of Q¹ is optionally substituted with one or more halo, C₁₋₆alkyl, C₂₋₆alkenyl, C₁₋₆alkoxy, —NH₂, or C₃₋₁₀cycloalkyl, wherein,
   the C₁₋₆alkyl is optionally substituted with one or more halo, and
   the C₃₋₁₀cycloalkyl is optionally substituted with one or more halo or C₁₋₆alkyl, and
(iv) C₃₋₁₀cycloalkyl;
or
(2) L¹ is —CH₂—; and
Q¹ is C₃₋₁₀cycloalkyl;
L² is —C(O)— or —S(O)₂—
R¹ is H or C₁₋₆alkyl;
R^{k} is H, halo, —OH, —NH₂, or —NH—C(O)C₁₋₆alkyl;
R^{m} is H, —OH, or C₁₋₆alkyl;
Rⁿ is H, C₁₋₆alkyl, or C₃₋₁₀cycloalkyl or Rⁿ taken together with the carbon atom to which it is attached forms C₃₋₅ cycloalkyl;
or R^{k} is taken together with either R^{m} or Rⁿ, and the atoms to which they are attached, to form cyclopropyl; and
R² is selected from (i) to (vii):
(i) C₁₋₆alkyl, wherein the C₁₋₆alkyl of R² is optionally substituted with one or more R^{a}, wherein R^{a} is:
   (a) —OH,
   (b) cyano,
   (c) C₂₋₆alkynyl,
   (d) C₆₋₂₀aryl, wherein the C₆₋₂₀aryl of R^{a} is optionally substituted with one or more halo, cyano, C₁₋₆alkoxy, or —NH—C(O)—C₁₋₆alkyl,
   (e) 3-15 membered heterocyclyl, wherein the 3-15 membered heterocyclyl of R^{a} is optionally substituted with one or more R^{c}, wherein
      R^{c} is halo, oxo, C₁₋₆alkyl, C₁₋₆alkoxy, —C(O)—C₁₋₆alkyl, or —C(O)—C₁₋₆alkoxy, wherein
         the C₁₋₆alkyl of R^{c} is optionally substituted with one or more halo or C₂₋₆alkynyl, and
         the —C(O)—C₁₋₆alkoxy of R^{c} is optionally substituted with one or more halo,
   (f) —N(R^{c})(R^{d}), wherein R^{c} and R^{d} of —N(R^{c})(R^{d}) are, independently of each other, H, C₁₋₆alkyl, —C(O)—C₁₋₆alkyl, —C(O)—C₁₋₆alkoxy, —C(O)—NH₂, —C(O)—NH(C₁₋₆alkyl), —C(O)—N(C₁₋₆alkyl)₂, —C(O)-(3-15 membered heterocyclyl), —CH₂—C(O)—NH₂, 3-15 membered heterocyclyl, or 5-20 membered heteroaryl, wherein
      the C₁₋₆alkyl of R^{c} or R^{d} is optionally substituted with one or more —C(O)—NH₂,
      the —C(O)—C₁₋₆alkyl of R^{c} or R^{d} is optionally substituted with one or more halo,
      the 3-15 membered heterocyclyl and the 5-20 membered heteroaryl of R^{c} or R^{d} are independently optionally substituted with one or more C₁₋₆alkyl,
      the —C(O)-(3-15 membered heterocyclyl) of R^{c} or R^{d} is optionally substituted with one or more halo, —C(O)—C₁₋₆alkoxy, or C₁₋₆alkyl, wherein the C₁₋₆alkyl is optionally substituted with one or more halo, C₁₋₆alkoxy, or C₃₋₁₀cycloalkyl, and
      the C₁₋₆alkyl of the —C(O)—N(C₁₋₆alkyl)₂ of R^{c} or R^{d} are, independently of each other, optionally substituted with one or more halo or C₆₋₂₀aryl,
   (g) —O—R^{e}, wherein R^{e} is C₁₋₆alkyl, C₆₋₂₀aryl, —C(O)-(3-15 membered heterocyclyl), —C(O)—N—(C₁₋₆alkyl)₂, or 5-20 membered heteroaryl, wherein
      the C₁₋₆alkyl of R^{e} is optionally substituted with one or more C₁₋₆alkoxy, wherein the C₁₋₆alkoxy is optionally substituted with one or more C₂₋₆alkynyl,
      the C₆₋₂₀aryl of R^{e} is optionally substituted with one or more C₁₋₆alkyl, and
      the —C(O)-(3-15 membered heterocyclyl) of R^{e} is optionally substituted with one or more C₁₋₆alkyl, C₁₋₆alkoxy, or —C(O)—C₁₋₆alkoxy, wherein the C₁₋₆alkyl is optionally substituted with one or more halo, C₁₋₆alkoxy, or C₃₋₁₀cycloalkyl,
   (h) —C(O)—R^{e}, wherein R^{e} is —NH₂, —OH, or 3-15 membered heterocyclyl, or
   (i) —S(O)₂—R^{f}, wherein R^{f} is C₁₋₆alkyl or 3-15 membered heterocyclyl,
(ii) C₃₋₁₀cycloalkyl, wherein the C₃₋₁₀cycloalkyl of R² is optionally substituted with one or more R^{q}, wherein R^{q} is 5-20 membered heteroaryl or C₆₋₂₀aryl, wherein the C₆₋₂₀aryl of R^{q} is optionally substituted with one or more C₁₋₆alkoxy,
(iii) 3-15 membered heterocyclyl, wherein the 3-15 membered heterocyclyl of R² is optionally substituted with one or more halo, oxo, C₁₋₆alkyl, —C(O)—C₁₋₆alkyl, or 5-20 membered heteroaryl,
(iv) 5-20 membered heteroaryl or —(C₁₋₄alkyl)(5-20 membered heteroaryl), wherein the C₁₋₄ alkyl is optionally substituted with one or more or more —OH, halo, —NH₂, —NH(C₁₋₆alkyl), —N(C₁₋₆alkyl)₂, and wherein the 5-20 membered heteroaryl of the 5-20 membered heteroaryl or —(C₁₋₄alkyl)(5-20 membered heteroaryl), is optionally substituted with one or more R^{s}, wherein
   R^{s} is halo, C₁₋₆alkyl, C₁₋₆alkoxy, —NH₂, —NH(C₁₋₆alkyl), —N(C₁₋₆alkyl)₂, —NH—C(O)—C₁₋₆alkyl, C₆₋₂₀aryl, C₃₋₁₀cycloalkyl, 3-15 membered heterocyclyl, 5-20 membered heteroaryl, or —C(O)—C₁₋₆alkoxy, wherein
      the C₁₋₆alkyl of R^{s} is optionally substituted with one or more halo, C₁₋₆alkoxy, —NH₂, —NH(C₁₋₆alkyl), —N(C₁₋₆alkyl)₂, —NH—C(O)C₁₋₆alkyl, or —NH—C(O)—C₁₋₆alkoxy, and
      the 3-15-membered heterocyclyl of R^{s} is optionally substituted with one or more halo or —C(O)—C₁₋₆alkoxy,
(v) —N(R^{g})(R^{h}), wherein R^{g} and R^{h} are independently H or C₁₋₆alkyl,
(vi) —C(O)—R^{j}, wherein R^{j} is C₃₋₁₀cycloalkyl, —NH(C₁₋₆alkyl), —N(C₁₋₆alkyl)₂, or —NH(5-20 membered heteroaryl), and
(vii) C₆₋₂₀aryl, wherein the C₆₋₂₀aryl of R² is optionally substituted with one or more 5-20 membered heteroaryl or —O—R^{p}, wherein R^{p} is 3-15 membered heterocyclyl, wherein the 3-15 membered heterocyclyl of R^{p} is optionally substituted with one or more —C(O)—C₁₋₆alkyl; and
wherein (G1-Z1) and (Z2-G2) are each independently substituted with a group bound to L.

In one aspect, provided herein is a compound of formula (I), or a stereoisomer or tautomer thereof, or a pharmaceutically acceptable salt of any of the foregoing, wherein (G1-Z1) and (G2-Z2) are each, independently, of formula (I-A):

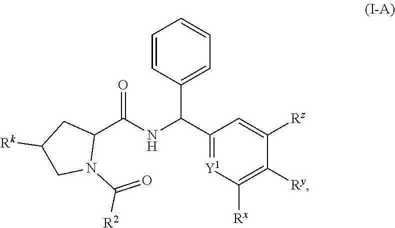

or a stereoisomer or tautomer thereof, or a pharmaceutically acceptable salt of any of the foregoing, wherein Y¹, R², R^{k}, R^{x}, R^{y}, and R^{z} are as defined elsewhere herein.

In one aspect, provided herein is a compound of formula (I), or a stereoisomer or tautomer thereof, or a pharmaceutically acceptable salt of any of the foregoing, wherein (G1-Z1) and (G2-Z2) are each, independently, of formula (I-B):

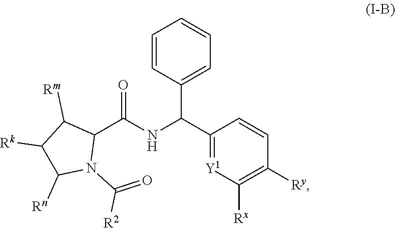

or a stereoisomer or tautomer thereof, or a pharmaceutically acceptable salt of any of the foregoing, wherein Y¹, R², R^{k}, R^{m}, Rⁿ, R^{x}, and R^{y} are as defined elsewhere herein.

In one aspect, provided herein is a compound of formula (I), or a stereoisomer or tautomer thereof, or a pharmaceutically acceptable salt of any of the foregoing, wherein (G1-Z1) and (G2-Z2) are each, independently, of formula (I-C):

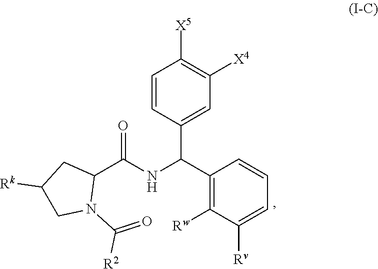

or a stereoisomer or tautomer thereof, or a pharmaceutically acceptable salt of any of the foregoing, wherein X⁴, X⁵, R², R^{k}, R^{v}, and R^{w} are as defined elsewhere herein.

In one aspect, provided herein is a compound of formula (I), or a stereoisomer or tautomer thereof, or a pharmaceutically acceptable salt of any of the foregoing, wherein (G1-Z1) and (G2-Z2) are each, independently, of formula (I-D):

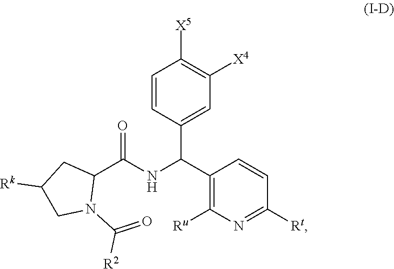

or a stereoisomer or tautomer thereof, or a pharmaceutically acceptable salt of any of the foregoing, wherein X⁴, X⁵, R², R^{k}, R^{u}, and R^{t} are as defined elsewhere herein.

In one aspect, provided herein is a compound of formula (I), or a stereoisomer or tautomer thereof, or a pharmaceutically acceptable salt of any of the foregoing, wherein (G1-Z1) and (G2-Z2) are each, independently, of formula (I-D1):

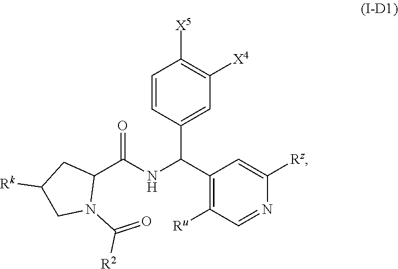

wherein X⁴, X⁵, R², R^{k}, R^{u}, and R^{z} of formula (I-D1) are as defined elsewhere herein.

In one aspect, provided herein is a compound of formula (I), or a stereoisomer or tautomer thereof, or a pharmaceutically acceptable salt of any of the foregoing, wherein (G1-Z1) and (G2-Z2) are each, independently, of formula (I-D2):

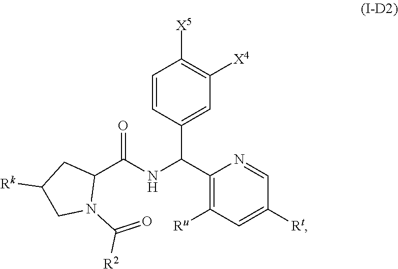

wherein X⁴, X⁵, R², R^{k}, R^{t}, and R^{u} of formula (I-D2) are as defined elsewhere herein.

In one aspect, provided herein is a compound of formula (I), or a stereoisomer or tautomer thereof, or a pharmaceutically acceptable salt of any of the foregoing, wherein (G1-Z1) and (G2-Z2) are each, independently, of formula (I-E):

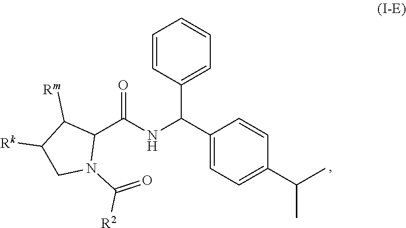

or a stereoisomer or tautomer thereof, or a pharmaceutically acceptable salt of any of the foregoing, wherein R², R^{k}, and R^{m} are as defined elsewhere herein.

In one aspect, provided herein is a compound of formula (I), or a stereoisomer or tautomer thereof, or a pharmaceutically acceptable salt of any of the foregoing, wherein (G1-Z1) and (G2-Z2) are each, independently, of formula (I-F):

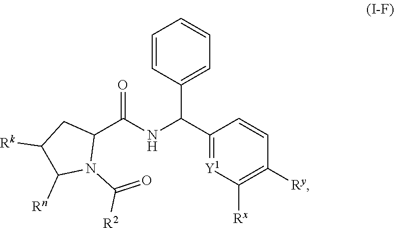

or a stereoisomer or tautomer thereof, or a pharmaceutically acceptable salt of any of the foregoing, wherein Y¹, R², R^{k}, Rⁿ, R^{x}, and R^{y} are as defined elsewhere herein.

In one aspect, provided herein is a compound of formula (I), or a stereoisomer or tautomer thereof, or a pharmaceutically acceptable salt of any of the foregoing, wherein (G1-Z1) and (G2-Z2) are each, independently, of formula (I-G):

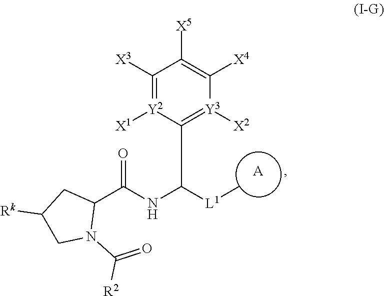

or a stereoisomer or tautomer thereof, or a pharmaceutically acceptable salt of any of the foregoing, wherein ring A, L¹, Y², Y³, R², R^{k}, X¹, X², X³, X⁴, and X⁵ are as defined elsewhere herein.

In one aspect, provided herein is a compound of formula (I), or a stereoisomer or tautomer thereof, or a pharmaceutically acceptable salt of any of the foregoing, wherein (G1-Z1) and (G2-Z2) are each, independently, of formula (I-H):

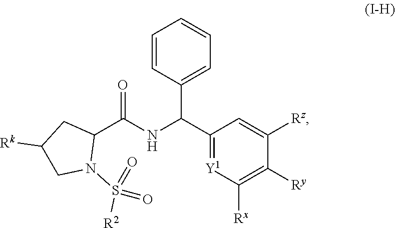

or a stereoisomer or tautomer thereof, or a pharmaceutically acceptable salt of any of the foregoing, wherein Y¹, R², R^{k}, Rⁿ, R^{x}, and R^{y} are as defined elsewhere herein.

In one aspect, provided herein is a compound of formula (I), wherein the compound is a compound of formula (II):

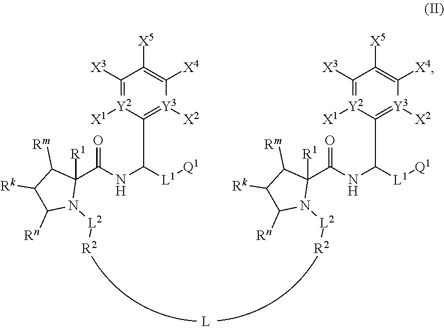

or a stereoisomer or tautomer thereof, or a pharmaceutically acceptable salt of any of the foregoing, wherein L, L¹, L², Y², Y³, R¹, R², R^{k}, R^{m}, Rⁿ, X¹, X², X³, X⁴, X⁵, and Q¹ are as defined elsewhere herein.

In one aspect, provided herein is a compound of formula (I), wherein the compound is a compound of formula (III):

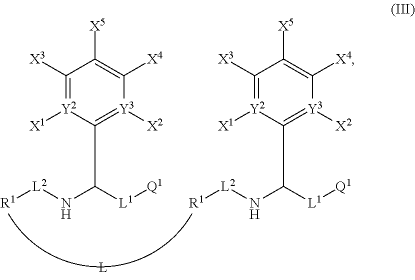

or a stereoisomer or tautomer thereof, or a pharmaceutically acceptable salt of any of the foregoing, wherein L, L¹, L², Y², Y³, R², X¹, X², X³, X⁴, X⁵, and Q¹ are as defined elsewhere herein.

In one aspect, provided herein is a compound of formula (I), wherein the compound is a compound of formula (IV):

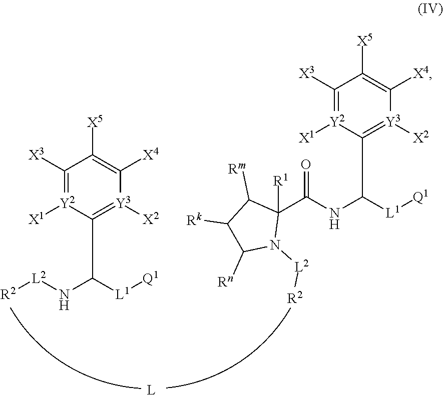

or a stereoisomer or tautomer thereof, or a pharmaceutically acceptable salt of any of the foregoing, wherein L, L¹, L², Y², Y³, R², R^{k}, R^{m}, Rⁿ, X¹, X², X³, X⁴, X⁵, and Q¹ are as defined elsewhere herein.

In one aspect, provided herein is a compound of formula (I), wherein the compound is a compound of formula (V):

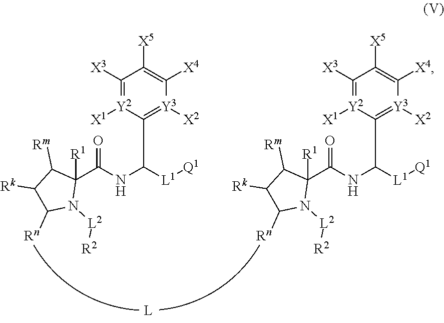

or a stereoisomer or tautomer thereof, or a pharmaceutically acceptable salt of any of the foregoing, wherein L, L¹, L², Y², Y³, R², R^{k}, R^{m}, Rⁿ, X¹, X², X³, X⁴, X⁵, and Q¹ are as defined elsewhere herein.

In one aspect, provided herein is a pharmaceutical composition, comprising (i) a compound of formula (I), or any variation or embodiment thereof, or a stereoisomer or tautomer thereof, or a pharmaceutically acceptable salt of any of the foregoing, and (ii) one or more pharmaceutically acceptable excipients.

In one aspect, provided herein is a method of modulating GYS1 in a cell, comprising exposing the cell to (i) a composition comprising an effective amount of a compound of formula (I), or any variation or embodiment thereof, or a stereoisomer or tautomer thereof, or a pharmaceutically acceptable salt of any of the foregoing, or (ii) a pharmaceutical composition, comprising a compound of formula (I), or any variation or embodiment thereof, or a stereoisomer or tautomer thereof, or a pharmaceutically acceptable salt of any of the foregoing, and one or more pharmaceutically acceptable excipients.

In one aspect, provided herein is a method of inhibiting GYS1 in a cell, comprising exposing the cell to (i) a composition comprising an effective amount of a compound of formula (I), or any variation or embodiment thereof, or a stereoisomer or tautomer thereof, or a pharmaceutically acceptable salt of any of the foregoing, or (ii) a pharmaceutical composition, comprising a compound of formula (I), or any variation or embodiment thereof, or a stereoisomer or tautomer thereof, or a pharmaceutically acceptable salt of any of the foregoing, and one or more pharmaceutically acceptable excipients.

In one aspect, provided herein is a method of reducing tissue glycogen stores in an individual in need thereof, comprising administering to the individual an effective amount of (i) a compound of formula (I), or (ii) a pharmaceutical composition comprising a compound of formula (I) and one or more pharmaceutically acceptable excipients. In some embodiments, the GYS1 inhibitor is selective for GYS1 over GYS2. In some embodiments, the compound of formula (I) is greater than 500 or 1,000 or 1,500 or 1,700-fold selective for GYS1 over GYS2.

In one aspect, provided herein is a method of reducing tissue glycogen stores in an individual in need thereof, comprising administering to the individual an effective amount of (i) a composition comprising an effective amount of a compound of formula (I), or any variation or embodiment thereof, or a stereoisomer or tautomer thereof, or a pharmaceutically acceptable salt of any of the foregoing, or (ii) a pharmaceutical composition, comprising a compound of formula (I), or any variation or embodiment thereof, or a stereoisomer or tautomer thereof, or a pharmaceutically acceptable salt of any of the foregoing, and one or more pharmaceutically acceptable excipients.

In one aspect, provided herein is a method of modulating GYS1 in a cell of an individual in need thereof, comprising administering to the individual an effective amount of (i) a composition comprising an effective amount of a compound of formula (I), or any variation or embodiment thereof, or a stereoisomer or tautomer thereof, or a pharmaceutically acceptable salt of any of the foregoing, or (ii) a pharmaceutical composition, comprising a compound of formula (I), or any variation or embodiment thereof, or a stereoisomer or tautomer thereof, or a pharmaceutically acceptable salt of any of the foregoing, and one or more pharmaceutically acceptable excipients.

In one aspect, provided herein is a method of treating a GYS1-mediated disease, disorder, or condition in an individual in need thereof, comprising subjecting the individual to glycogen substrate reduction therapy. In some embodiments, the glycogen substrate reduction therapy comprises administration of a compound of formula (I). In some embodiments, the compound of formula (I) is selective for GYS1 over GYS2. In some embodiments, the compound of formula (I) is greater than 500 or 1,000 or 1,500 or 1,700-fold selective for GYS1 over GYS2.

In one aspect, provided herein is a method of treating a GYS1-mediated disease, disorder, or condition in an individual in need thereof, comprising administering to the individual an effective amount of (i) a composition comprising an effective amount of a compound of formula (I), or any variation or embodiment thereof, or a stereoisomer or tautomer thereof, or a pharmaceutically acceptable salt of any of the foregoing, or (ii) a pharmaceutical composition, comprising a compound of formula (I), or any variation or embodiment thereof, or a stereoisomer or tautomer thereof, or a pharmaceutically acceptable salt of any of the foregoing, and one or more pharmaceutically acceptable excipients.

In one aspect, provided herein is a method of treating a glycogen storage disease, disorder, or condition in an individual in need thereof, comprising subjecting the individual to glycogen substrate reduction therapy. In some embodiments, the glycogen substrate reduction therapy comprises administration of a compound of formula (I). In some embodiments, the compound of formula (I) is selective for GYS1 over GYS2. In some embodiments, the compound of formula (I) is greater than 500 or 1,000 or 1,500 or 1,700-fold selective for GYS1 over GYS2.

In one aspect, provided herein is a kit, comprising (i) a composition comprising an effective amount of a compound of formula (I), or any variation or embodiment thereof, or a stereoisomer or tautomer thereof, or a pharmaceutically acceptable salt of any of the foregoing, or a pharmaceutical composition, comprising a compound of formula (I), or any variation or embodiment thereof, or a stereoisomer or tautomer thereof, or a pharmaceutically acceptable salt of any of the foregoing, and one or more pharmaceutically acceptable excipients, and (ii) instructions for use in treating an GYS1-mediated disease, disorder, or condition in an individual in need thereof.

In some aspect, provided herein are methods of preparing a compound of formula (I), or any embodiment or variation thereof, such as a compound of formula (II), (II-A), (III), (II-A), (IV), (IV-A), (V), or (V-A), or a stereoisomer or tautomer thereof, or a pharmaceutically acceptable salt of any of the foregoing.

### BRIEF DESCRIPTION OF THE DRAWINGS

FIG. 1 depicts the pathway in which PPP1R3A Loss of Function (LoF) leads to reduction in muscle glycogen.

FIGS. 2A and 2B depict the association between PPP1R3A protein truncating variant (PTV) and left ventricular ejection (LVEF) (%) and left ventricle wall thickness (mm) in UK Biobank.

FIGS. 2C and 2D depict the association between PPP1R3A protein truncating variant (PTV) and exercise output (watts) and max heart rate (HR) exercise (bpm) in UK Biobank.

FIGS. 2E and 2F depict the association between PPP1R3A protein truncating variant (PTV) and PQ interval (ms) and QRS duration (ms) in UK Biobank.

FIGS. 2G and 2H depict the association between PPP1R3A protein truncating variant (PTV) and QT interval (ms) and serum glucose (mmol/L) in UK Biobank.

### DETAILED DESCRIPTION OF THE INVENTION

“Individual” refers to mammals and includes humans and non-human mammals. Examples of individuals include, but are not limited to, mice, rats, hamsters, guinea pigs, pigs, rabbits, cats, dogs, goats, sheep, cows, and humans. In some embodiments, individual refers to a human.

As used herein, “about” a parameter or value includes and describes that parameter or value per se. For example, “about X” includes and describes X per se.

As used herein, an “at risk” individual is an individual who is at risk of developing a disease or condition. An individual “at risk” may or may not have a detectable disease or condition, and may or may not have displayed detectable disease prior to the treatment methods described herein. “At risk” denotes that an individual has one or more so-called risk factors, which are measurable parameters that correlate with development of a disease or condition and are known in the art. An individual having one or more of these risk factors has a higher probability of developing the disease or condition than an individual without these risk factor(s).

“Treatment” or “treating” is an approach for obtaining beneficial or desired results including clinical results. Beneficial or desired results may include one or more of the following: decreasing one or more symptom resulting from the disease or condition; diminishing the extent of the disease or condition; slowing or arresting the development of one or more symptom associated with the disease or condition (e.g., stabilizing the disease or condition, preventing or delaying the worsening or progression of the disease or condition); and relieving the disease, such as by causing the regression of clinical symptoms (e.g., ameliorating the disease state, enhancing the effect of another medication, delaying the progression of the disease, increasing the quality of life, and/or prolonging survival).

As used herein, “delaying” development of a disease or condition means to defer, hinder, slow, retard, stabilize and/or postpone development of the disease or condition. This delay can be of varying lengths of time, depending on the history of the disease and/or individual being treated. As is evident to one skilled in the art, a sufficient or significant delay can, in effect, encompass prevention, in that the individual does not develop the disease or condition.

As used herein, the term “therapeutically effective amount” or “effective amount” intends such amount of a compound of the disclosure or a pharmaceutically salt thereof sufficient to effect treatment when administered to an individual. As is understood in the art, an effective amount may be in one or more doses, e.g., a single dose or multiple doses may be required to achieve the desired treatment endpoint. An effective amount may be considered in the context of administering one or more therapeutic agents, and a single agent may be considered to be given in an effective amount if, in conjunction with one or more other agents, a desirable or beneficial result may be or is achieved.

As used herein, “unit dosage form” refers to physically discrete units, suitable as unit dosages, each unit containing a predetermined quantity of active ingredient, or compound, which may be in a pharmaceutically acceptable carrier.

As used herein, by “pharmaceutically acceptable” is meant a material that is not biologically or otherwise undesirable, e.g., the material may be incorporated into a pharmaceutical composition administered to an individual without causing significant undesirable biological effects.

The term “alkyl”, as used herein, refers to an unbranched or branched saturated univalent hydrocarbon chain. As used herein, alkyl has 1-20 carbons (i.e., C₁₋₂₀alkyl), 1-16 carbons (i.e., C₁₋₆alkyl), 1-12 carbons (i.e., C₁₋₁₂alkyl), 1-10 carbons (i.e., C₁₋₁₀alkyl), 1-8 carbons (i.e., C₁₋₈alkyl), 1-6 carbons (i.e., C₁₋₆alkyl), 1-4 carbons (i.e., C₁₋₄alkyl), or 1-3 carbons (i.e., C₁₋₃alkyl). Examples of alkyl groups include, but are not limited to, methyl, ethyl, propyl, iso-propyl, n-butyl, sec-butyl, iso-butyl, tert-butyl, pentyl, 2-pentyl, iso-pentyl, neo-pentyl, hexyl, 2-hexyl, 3-hexyl, and 3-methylpentyl. When an alkyl residue having a specific number of carbons is named by chemical name or molecular formula, all positional isomers having that number of carbon atoms may be encompassed—for example, “butyl” includes n-butyl, sec-butyl, iso-butyl, and tert-butyl; and “propyl” includes n-propyl and iso-propyl. Certain commonly used alternative names may be used and will be understood by those of ordinary skill in the art. For instance, a divalent group, such as a divalent “alkyl” group, may be referred to as an “alkylene”.

The term “alkenyl”, as used herein, refers to a branched or unbranched univalent hydrocarbon chain comprising at least one carbon-carbon double bond. As used herein, alkenyl has 2-20 carbons (i.e., C₂₋₂₀alkenyl), 2-16 carbons (i.e., C₂₋₁₆alkenyl), 2-12 carbons (i.e., C₂₋₁₂alkenyl), 2-10 carbons (i.e., C₂₋₁₀alkenyl), 2-8 carbons (i.e., C₂₋₈alkenyl), 2-6 carbons (i.e., C₂₋₆alkenyl), 2-4 carbons (i.e., C₂₋₄alkenyl), or 2-3 carbons (i.e., C₂₋₃alkenyl). Examples of alkenyl include, but are not limited to, ethenyl, prop-1-enyl, prop-2-enyl 1,2-butadienyl, and 1,3-butadienyl. When an alkenyl residue having a specific number of carbons is named by chemical name or molecular formula, all positional isomers having that number of carbon atoms may be encompassed—for example, “propenyl” includes prop-1-enyl and prop-2-enyl. Certain commonly used alternative names may be used and will be understood by those of ordinary skill in the art. For instance, a divalent group, such as a divalent “alkenyl” group, may be referred to as an “alkenylene”.

The term “alkynyl”, as used herein, refers to a branched or unbranched univalent hydrocarbon chain comprising at least one carbon-carbon triple bond. As used herein, alkynyl has 2-20 carbons (i.e., C₂₋₂₀alkynyl), 2-16 carbons (i.e., C₂₋₁₆alkynyl), 2-12 carbons (i.e., C₂₋₁₂alkynyl), 2-10 carbons (i.e., C₂₋₁₀alkynyl), 2-8 carbons (i.e., C₂₋₈alkynyl), 2-6 carbons (i.e., C₂₋₆alkynyl), 2-4 carbons (i.e., C₂₋₄alkynyl), or 2-3 carbons (i.e., C₂₋₃alkynyl). Examples of alkynyl include, but are not limited to, ethynyl, prop-1-ynyl, prop-2-ynyl, but-1-ynyl, but-2-ynyl, and but-3-ynyl. When an alkynyl residue having a specific number of carbons is named by chemical name or molecular formula, all positional isomers having that number of carbon atoms may be encompassed—for example, “propynyl” includes prop-1-ynyl and prop-2-ynyl. Certain commonly used alternative names may be used and will be understood by those of ordinary skill in the art. For instance, a divalent group, such as a divalent “alkynyl” group, may be referred to as an “alkynylene”.

The term “alkoxy”, as used herein, refers to an —O-alkyl moiety. Examples of alkoxy groups include, but are not limited to, methoxy, ethoxy, n-propoxy, iso-propoxy, n-butoxy, tert-butoxy, sec-butoxy, n-pentoxy, n-hexoxy, and 1,2-dimethylbutoxy.

The term “aryl”, as used herein, refers to a fully unsaturated carbocyclic ring moiety. The term “aryl” encompasses monocyclic and polycyclic fused-ring moieties. As used herein, aryl encompasses ring moieties comprising, for example, 6 to 20 annular carbon atoms (i.e., C₆₋₂₀aryl), 6 to 16 annular carbon atoms (i.e., C₆₋₁₆aryl), 6 to 12 annular carbon atoms (i.e., C₆₋₁₂aryl), or 6 to 10 annular carbon atoms (i.e., C₆₋₁₀aryl). Examples of aryl moieties include, but are not limited to, phenyl, naphthyl, fluorenyl, and anthryl.

The term “cycloalkyl”, as used herein, refers to a saturated or partially unsaturated carbocyclic ring moiety. The term “cycloalkyl” encompasses monocyclic and polycyclic ring moieties, wherein the polycyclic moieties may be fused, branched, or spiro. Cycloalkyl includes cycloalkenyl groups, wherein the ring moiety comprises at least one annular double bond.

Cycloalkyl includes any polycyclic carbocyclic ring moiety comprising at least one non-aromatic ring, regardless of the point of attachment to the remainder of the molecule. As used herein, cycloalkyl includes rings comprising, for example, 3 to 20 annular carbon atoms (i.e., a C₃₋₂₀cycloalkyl), 3 to 16 annular carbon atoms (i.e., a C₃₋₁₆cycloalkyl), 3 to 12 annular carbon atoms (i.e., a C₃₋₁₂cycloalkyl), 3 to 10 annular carbon atoms (i.e., a C₃₋₁₀cycloalkyl), 3 to 8 annular carbon atoms (i.e., a C₃₋₈cycloalkyl), 3 to 6 annular carbon atoms (i.e., a C₃₋₆cycloalkyl), or 3 to 5 annular carbon atoms (i.e., a C₃₋₅cycloalkyl). Monocyclic cycloalkyl ring moieties include, for example, cyclopropyl, cyclobutyl, cyclopentyl, cyclohexyl, cycloheptyl, and cyclooctyl. Polycyclic groups include, for example, bicyclo[2.2.1]heptanyl, bicyclo[2.2.2]octanyl, adamantyl, norbornyl, decalinyl, 7,7-dimethyl-bicyclo[2.2.1]heptanyl, and the like. Still further, cycloalkyl also includes spiro cycloalkyl ring moieties, for example, spiro[2.5]octanyl, spiro[4.5]decanyl, or spiro [5.5]undecanyl.

The term “halo”, as used herein, refers to atoms occupying groups VIIA of The Periodic Table and includes fluorine (fluoro), chlorine (chloro), bromine (bromo), and iodine (iodo).

The term “heteroaryl”, as used herein, refers to an aromatic (fully unsaturated) ring moiety that comprises one or more annular heteroatoms independently selected from the group consisting of nitrogen, oxygen, and sulfur. The term “heteroaryl” includes both monocyclic and polycyclic fused-ring moieties. As used herein, a heteroaryl comprises, for example, 5 to 20 annular atoms (i.e., a 5-20 membered heteroaryl), 5 to 16 annular atoms (i.e., a 5-16 membered heteroaryl), 5 to 12 annular atoms (i.e., a 5-12 membered heteroaryl), 5 to 10 annular atoms (i.e., a 5-10 membered heteroaryl), 5 to 8 annular atoms (i.e., a 5-8 membered heteroaryl), or 5 to 6 annular atoms (i.e., a 5-6 membered heteroaryl). Any monocyclic or polycyclic aromatic ring moiety comprising one or more annular heteroatoms is considered a heteroaryl, regardless of the point of attachment to the remainder of the molecule (i.e., the heteroaryl moiety may be attached to the remainder of the molecule through any annular carbon or any annular heteroatom of the heteroaryl moiety). Examples of heteroaryl groups include, but are not limited to, acridinyl, benzimidazolyl, benzothiazolyl, benzindolyl, benzofuranyl, benzothiazolyl, benzothiadiazolyl, benzonaphthofuranyl, benzoxazolyl, benzothienyl (benzothiophenyl), benzotriazolyl, benzo[4,6]imidazo[1,2-a]pyridyl, carbazolyl, cinnolinyl, dibenzofuranyl, dibenzothiophenyl, furanyl, isothiazolyl, imidazolyl, indazolyl, indolyl, indazolyl, isoindolyl, isoquinolyl, isoxazolyl, naphthyridinyl, oxadiazolyl, oxazolyl, 1-oxidopyridinyl, 1-oxidopyrimidinyl, 1-oxidopyrazinyl, 1-oxidopyridazinyl, phenazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, thiazolyl, thiadiazolyl, triazolyl, tetrazolyl, and triazinyl. Examples of the fused-heteroaryl rings include, but are not limited to, benzo[d]thiazolyl, quinolinyl, isoquinolinyl, benzo[b]thiophenyl, indazolyl, benzo[d]imidazolyl, pyrazolo[1,5-a]pyridinyl, and imidazo[1,5-a]pyridinyl, wherein the heteroaryl can be bound via either ring of the fused system.

The term “heterocyclyl”, as used herein, refers to a saturated or partially unsaturated cyclic moiety that encompasses one or more annular heteroatoms independently selected from the group consisting of nitrogen, oxygen, and sulfur. The term “heterocyclyl” includes both monocyclic and polycyclic ring moieties, wherein the polycyclic ring moieties may be fused, bridged, or spiro. Any non-aromatic monocyclic or polycyclic ring moiety comprising at least one annular heteroatom is considered a heterocyclyl, regardless of the point of attachment to the remainder of the molecule (i.e., the heterocyclyl moiety may be attached to the remainder of the molecule through any annular carbon or any annular heteroatom of the heterocyclyl moiety). Further, the term heterocyclyl is intended to encompass any polycyclic ring moiety comprising at least one annular heteroatom wherein the polycyclic ring moiety comprises at least one non-aromatic ring, regardless of the point of attachment to the remainder of the molecule. As used herein, a heterocyclyl comprises, for example, 3 to 20 annular atoms (i.e., a 3-20 membered heterocyclyl), 3 to 16 annular atoms (i.e., a 3-16 membered heterocyclyl), 3 to 12 annular atoms (i.e., a 3-12 membered heterocyclyl), 3 to 10 annular atoms (i.e., a 3-10 membered heterocyclyl), 3 to 8 annular atoms (i.e., a 3-8 membered heterocyclyl), 3 to 6 annular atoms (i.e., a 3-6 membered heterocyclyl), 3 to 5 annular atoms (i.e., a 3-5 membered heterocyclyl), 5 to 8 annular atoms (i.e., a 5-8 membered heterocyclyl), or 5 to 6 annular atoms (i.e., a 5-6 membered heterocyclyl). Examples of heterocyclyl groups include, e.g., azetidinyl, azepinyl, benzodioxolyl, benzo[b][1,4]dioxepinyl, 1,4-benzodioxanyl, benzopyranyl, benzodioxinyl, benzopyranonyl, benzofuranonyl, dioxolanyl, dihydropyranyl, hydropyranyl, thienyl[1,3]dithianyl, decahydroisoquinolyl, furanonyl, imidazolinyl, imidazolidinyl, indolinyl, indolizinyl, isoindolinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, oxiranyl, oxetanyl, phenothiazinyl, phenoxazinyl, piperidinyl, piperazinyl, 4-piperidonyl, pyrrolidinyl, pyrazolidinyl, quinuclidinyl, thiazolidinyl, tetrahydrofuryl, tetrahydropyranyl, trithianyl, tetrahydroquinolinyl, thiophenyl (i.e., thienyl), thiomorpholinyl, thiamorpholinyl, 1-oxo-thiomorpholinyl, and 1,1-dioxo-thiomorpholinyl. Examples of spiro heterocyclyl rings include, but are not limited to, bicyclic and tricyclic ring systems, such as oxabicyclo[2.2.2]octanyl, 2-oxa-7-azaspiro[3.5]nonanyl, 2-oxa-6-azaspiro[3.4]octanyl, and 6-oxa-1-azaspiro[3.3]heptanyl. Examples of fused heterocyclyl rings include, but are not limited to, 1,2,3,4-tetrahydroisoquinolinyl, 4,5,6,7-tetrahydrothieno[2,3-c]pyridinyl, indolinyl, and isoindolinyl, where the heterocyclyl can be bound via either ring of the fused system.

The term “oxo”, as used herein, refers to a ═O moiety.

The terms “optional” and “optionally”, as used herein, mean that the subsequently described event or circumstance may or may not occur and that the description includes instances where the event or circumstance occurs and instances where it does not. Accordingly, the term “optionally substituted” infers that any one or more (e.g., 1, 2, 1 to 5, 1 to 3, 1 to 2, etc.) hydrogen atoms on the designated atom or moiety or group may be replaced or not replaced by an atom or moiety or group other than hydrogen. By way of illustration and not limitation, the phrase “methyl optionally substituted with one or more chloro” encompasses —CH₃, —CH₂Cl, —CHCl₂, and —CCl₃ moieties.

It is understood that aspects and embodiments described herein as “comprising” include “consisting of” and “consisting essentially of” embodiments.

The term “pharmaceutically acceptable salt”, as used herein, of a given compound refers to salts that retain the biological effectiveness and properties of the given compound and which are not biologically or otherwise undesirable. “Pharmaceutically acceptable salts” include, for example, salts with inorganic acids, and salts with an organic acid. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. See, e.g., Handbook of Pharmaceutical Salts Properties, Selection, and Use, International Union of Pure and Applied Chemistry, John Wiley & Sons (2008), which is incorporated herein by reference. Those skilled in the art will recognize various synthetic methodologies that may be used to prepare nontoxic pharmaceutically acceptable addition salts. Pharmaceutically acceptable acid addition salts may be prepared from inorganic or organic acids. Salts derived from inorganic acids include, e.g., hydrochloric acid, hydrobromic acid, sulfuric acid, nitric acid, phosphoric acid, and the like. Salts derived from organic acids include, e.g., acetic acid, propionic acid, gluconic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, trifluoroacetic acid, and the like. Likewise, pharmaceutically acceptable base addition salts can be prepared from inorganic or organic bases. Salts derived from inorganic bases include, by way of example only, sodium, potassium, lithium, aluminum, ammonium, calcium, and magnesium salts. Salts derived from organic bases include, but are not limited to, salts of primary, secondary, and tertiary amines. Specific examples of suitable amines include, by way of example only, isopropylamine, trimethyl amine, diethyl amine, tri(iso-propyl) amine, tri(n-propyl) amine, ethanolamine, 2-dimethylaminoethanol, piperazine, piperidine, morpholine, N-ethylpiperidine, and the like.

Isotopically labeled forms of the compounds depicted herein may be prepared. Isotopically labeled compounds have structures depicted herein, except that one or more atoms are replaced by an atom having a selected atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. In some embodiments, a compound of formula (A) is provided wherein one or more hydrogen is replaced by deuterium or tritium.

Some of the compounds provided herein may exist as tautomers. Tautomers are in equilibrium with one another. By way of illustration, amide containing compounds may exist in equilibrium with imidic acid tautomers. Regardless of which tautomer is shown and regardless of the nature of the equilibrium among tautomers, the compounds of this disclosure are understood by one of ordinary skill in the art to comprise both amide and imidic acid tautomers. Thus, for example, amide-containing compounds are understood to include their imidic acid tautomers. Likewise, imidic-acid containing compounds are understood to include their amide tautomers.

Also provided herein are prodrugs of the compounds depicted herein, or a pharmaceutically acceptable salt thereof. Prodrugs are compounds that may be administered to an individual and release, in vivo, a compound depicted herein as the parent drug compound. It is understood that prodrugs may be prepared by modifying a functional group on a parent drug compound in such a way that the modification is cleaved in vitro or in vivo to release the parent drug compound. See, e.g., Rautio, J., Kumpulainen, H., Heimbach, T. et al. Prodrugs: design and clinical applications. Nat Rev Drug Discov 7, 255-270 (2008), which is incorporated herein by reference.

The compounds of the present disclosure, or their pharmaceutically acceptable salts, may include an asymmetric center and may thus give rise to enantiomers, diastereomers, and other stereoisomeric forms that may be defined, in terms of absolute stereochemistry, as (R)- or (S)- (or as (D)- or (L)- for amino acids). The present disclosure is meant to include all such possible isomers, as well as their racemic and optically pure forms and mixtures thereof in any ratio. Optically active (+) and (−), (R)- and (S)-, or (D)- and (L)-isomers may be prepared using chiral synthons or chiral reagents, or may be resolved using conventional techniques, for example, chromatography and/or fractional crystallization. Conventional techniques for the preparation/isolation of individual enantiomers include chiral synthesis from a suitable optically pure precursor or the resolution of the racemate (or the racemate of a salt or derivative) using, for example, chiral high pressure liquid chromatography (HPLC), and chiral supercritical fluid chromatography (SFC). When the compounds described herein contain olefinic double bonds or other centers of geometric asymmetry, unless specified otherwise, it is intended that the present disclosure includes both E and Z geometric isomers. Likewise, cis- and trans- are used in their conventional sense to describe relative spatial relationships.

A “stereoisomer” refers to a compound made up of the same atoms bonded by the same bonds, but having different three-dimensional structures, which are not interchangeable. The present disclosure contemplates various stereoisomers, or mixtures thereof, and includes “enantiomers,” which refers to two stereoisomers whose structures are non-superimposable mirror images of one another. “Diastereomers” are stereoisomers that have at least two asymmetric atoms, but which are not mirror images of each other.

Where enantiomeric and/or diastereomeric forms exist of a given structure, flat bonds indicate that all stereoisomeric forms of the depicted structure may be present, e.g.,

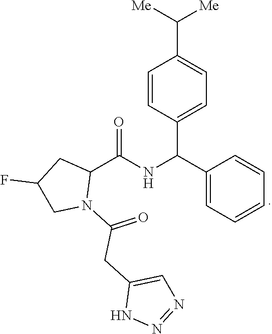 [SMILES: CC(C)c1ccc(C(NC(=O)C2CC(F)CN2C(=O)Cc2cnn[nH]2)c2ccccc2)cc1]

Where enantiomeric and/or diastereomeric forms exist of a given structure, flat bonds and the presence of a “*” symbol indicate that the composition is made up of at least 90%, by weight, of a single isomer with unknown stereochemistry, e.g.,

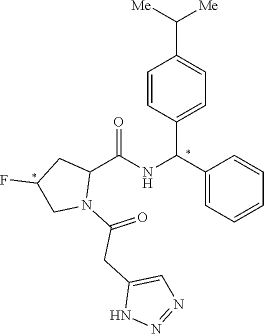 [SMILES: CC(C)c1ccc(C(NC(=O)C2CC(F)CN2C(=O)Cc2cnn[nH]2)c2ccccc2)cc1]

Where enantiomeric and/or diastereomeric forms exist of a given structure, wedged or hashed bonds indicate the composition is made up of at least 90%, by weight, of a single enantiomer or diastereomer with known stereochemistry, e.g.,

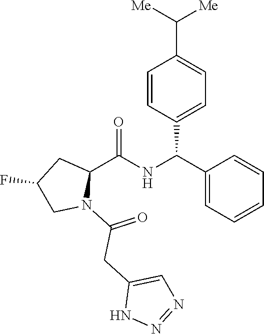 [SMILES: CC(C)c1ccc([C@@H](NC(=O)[C@@H]2C[C@@H](F)CN2C(=O)Cc2cnn[nH]2)c2ccccc2)cc1]

Where relevant, combinations of the above notation may be used. Exemplified species may contain stereogenic centers with known stereochemistry and stereogenic centers with unknown stereochemistry, stereochemistry, e.g.,

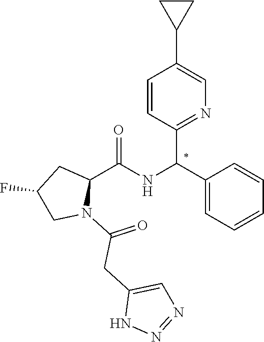 [SMILES: O=C(NC(c1ccccc1)c1ccc(C2CC2)cn1)[C@@H]1C[C@@H](F)CN1C(=O)Cc1cnn[nH]1]

Where relevant, combinations of the above notation may be used. Exemplified species may contain stereogenic centers with known stereochemistry and stereogenic centers bearing a mixture of isomers, e.g.,

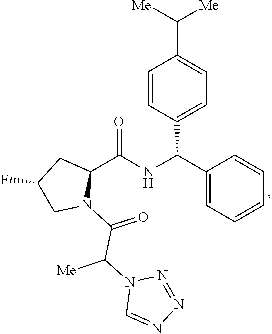 [SMILES: CC(C)c1ccc([C@@H](NC(=O)[C@@H]2C[C@@H](F)CN2C(=O)C(C)n2cnnn2)c2ccccc2)cc1]

### Compounds

In one aspect, provided herein is a compound of formula (I):

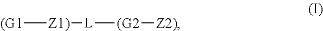

or a stereoisomer or tautomer thereof, or a pharmaceutically acceptable salt of any of the foregoing, wherein:
(i) G1 and/or G1-Z1, and (ii) G2 and/or G2-Z2 are each, independently, a GYS1 inhibiting moiety; and L is a linker.

In some embodiments of a compound of formula (I), or a stereoisomer or tautomer thereof, or a pharmaceutically acceptable salt of any of the foregoing, L is selected from the group consisting of a bond or (C₁-C₅₀)alkyl, wherein
the (C₁-C₅₀)alkyl is optionally substituted with one or more deutero, halo, (C₁-C₆)alkyl, (C₁-C₆)haloalkyl, oxo (C═O), —O(C₁-C₆)alkyl, —O(C₁-C₆)haloalkyl, —O(C₁-C₆)cycloalkyl, —N(R^{x})(R^{y})—, C₃-C₁₅ carbocyclcyl, 3- to 15-membered heterocyclcyl, C₆-C₁₄ aryl, or 5- to 20-membered heteroaryl; and
one or more of the C atoms in the (C₁-C₅₀)alkyl is optionally replaced with one or more —C(R^{x})═C(R^{x})—, —C≡C—, —O—, —C(O)—, —C(O)O—, —OC(O)—, —OC(O)O—, —OC(O)N(R^{x})—, —N(R^{x})C(O)O—, —N(R^{x})C(O)N(R^{y})—, —C(O)N(R^{x})—, —N(R^{x})C(O)—, —N(R^{x})—, —S(O)ₙ—, —S(O)ₙO—, —OS(O)ₙ—, —OS(O)ₙO—, —OS(O)ₙN(R^{x})—, —N(R^{x})S(O)ₙO—, —N(R^{x})S(O)ₙN(R^{y})—, —S(O)ₙN(R^{x})—, —N(R^{k})S(O)ₙ—, C₃-C₁₅ carbocyclcyl, 3- to 15-membered heterocyclcyl, C₆-C₁₄ aryl, or 5- to 20-membered heteroaryl;
wherein,
   each C₃-C₁₅ carbocyclcyl, 3- to 15-membered heterocyclcyl, C₆-C₁₄ aryl, or 5- to 20-membered heteroaryl is optionally substituted with one or more deutero, halo, (C₁-C₆)alkyl, (C₁-C₆)haloalkyl, oxo (C═O), —O(C₁-C₆)alkyl, —O(C₁-C₆)haloalkyl, —O(C₁-C₆)cycloalkyl, —N(R^{xx})(R^{yy}), —C(O)R^{z}, —C(O)OR^{z}, —OC(O)R^{z}, —OC(O)OR^{z}, —OC(O)N(R^{xx})(R^{yy}), —N(R^{xx})C(O)OR^{z}, —N(R^{xx})C(O)N((R^{xx})(R^{yy}), —C(O)N(R^{xx})(R^{yy}), —N(R^{xx})C(O)R^{z}, —N(R^{xx})(R^{yy}), —S(O)ₙR^{z}, —S(O)ₙOR^{z}, —OS(O)ₙR^{z}, —OS(O)ₙOR^{z}, —OS(O)ₙN(R^{xx})(R^{yy}), —N(R^{xx})S(O)ₙOR^{z}, —N(R^{xx})S(O)ₙN(R^{xx})(R^{yy}), —S(O)ₙN(R^{xx})(R^{yy}), or —N(R^{xx})S(O)ₙR^{z};
   each R^{x}, R^{xx}, R^{y}, R^{yy}, and R^{z} are independently H, (C₁-C₆)alkyl, (C₁-C₆)haloalkyl, or (C₁-C₆)cycloalkyl; and
   each n is independently 0-2.

In some embodiments of a compound of formula (I), or a stereoisomer or tautomer thereof, or a pharmaceutically acceptable salt of any of the foregoing, L is (C₁-C₅₀)alkyl optionally substituted with one or more deutero, halo, (C₁-C₆)alkyl, (C₁-C₆)haloalkyl, oxo (C═O), —O(C₁-C₆)alkyl, —O(C₁-C₆)haloalkyl, —O(C₁-C₆)cycloalkyl, —N(R^{x})(R^{y})—, C₃-C₁₅ carbocyclcyl, 3- to 15-membered heterocyclcyl, C₆-C₁₄ aryl, or 5- to 20-membered heteroaryl, wherein
one or more of the C atoms of the (C₁-C₅₀)alkyl is optionally replaced with one or more —C(R^{x})═C(R^{x})—, —C≡C—, —O—, —C(O)—, —N(R^{x})C(O)N(R^{y})—, —C(O)N(R^{x})—, —N(R^{x})C(O)—, —N(R^{x})—, —S(O)ₙ—, —N(R^{x})S(O)ₙN(R^{y})—, —S(O)ₙN(R^{x})—, —N(R^{x})S(O)ₙ—, C₃-C₁₅ carbocyclcyl, 3- to 15-membered heterocyclcyl, C₆-C₁₄ aryl, or 5- to 20-membered heteroaryl;
wherein
   each C₃-C₁₅ carbocyclcyl, 3- to 15-membered heterocyclcyl, C₆-C₁₄ aryl, and 5- to 20-membered heteroaryl are optionally substituted with one or more deutero, halo, (C₁-C₆)alkyl, (C₁-C₆)haloalkyl, oxo (C═O), —O(C₁-C₆)alkyl, —O(C₁-C₆)haloalkyl, —O(C₁-C₆)cycloalkyl, —N(R^{xx})(R^{yy}), —C(O)R^{z}, —C(O)OR^{z}, —OC(O)R^{z}, —N(R^{xx})C(O)N((R^{xx})(R^{yy}), —C(O)N(R^{xx})(R^{yy}), —N(R^{xx})(R^{yy}), —S(O)ₙR^{z}, —N(R^{xx})S(O)ₙN(R^{xx})(R^{yy}), —S(O)ₙN(R^{xx})(R^{yy}), or —N(R^{xx})S(O)ₙR^{z};
   each R^{x}, R^{xx}, R^{y}, R^{yy}, and R^{z} are independently H, (C₁-C₆)alkyl, (C₁-C₆)haloalkyl, or (C₁-C₆)cycloalkyl; and
   each n is independently 0-2.

In some embodiments of a compound of formula (I), or a stereoisomer or tautomer thereof, or a pharmaceutically acceptable salt of any of the foregoing, L is (C₁-C₂₀)alkyl, wherein
the (C₁-C₂₀)alkyl is optionally substituted with one or more deutero, halo, (C₁-C₆)alkyl, (C₁-C₆)haloalkyl, oxo (C═O), —O(C₁-C₆)alkyl, —O(C₁-C₆)haloalkyl, —O(C₁-C₆)cycloalkyl, —N(R^{x})(R^{y})—, C₃-C₁₅ carbocyclcyl, 3- to 15-membered heterocyclcyl, C₆-C₁₄ aryl, or 5- to 20-membered heteroaryl; and
one or more of the C atoms of the (C₁-C₂₀)alkyl is optionally replaced with one or more —C(R^{x})═C(R^{x})—, —C≡C—, —O—, —C(O)—, —N(R^{x})C(O)N(R^{y})—, —C(O)N(R^{x})—, —N(R^{x})C(O)—, —N(R^{x})—, —S(O)ₙ—, —N(R^{x})S(O)ₙN(R^{y})—, —S(O)ₙN(R^{x})—, —N(R^{x})S(O)ₙ—, C₃-C₁₅ carbocyclcyl, 3- to 15-membered heterocyclcyl, C₆-C₁₄ aryl, or 5- to 20-membered heteroaryl;
wherein
   each C₃-C₁₅ carbocyclcyl, 3- to 15-membered heterocyclcyl, C₆-C₁₄ aryl, and 5- to 20-membered heteroaryl are optionally substituted with one or more deutero, halo, (C₁-C₆)alkyl, (C₁-C₆)haloalkyl, oxo (C═O), —O(C₁-C₆)alkyl, —O(C₁-C₆)haloalkyl, —O(C₁-C₆)cycloalkyl, —N(R^{xx})(R^{yy}), —C(O)R^{z}, —C(O)OR^{z}, —OC(O)R^{z}, —N(R^{xx})C(O)N((R^{xx})(R^{yy}), —C(O)N(R^{xx})(R^{yy}), —N(R^{xx})(R^{yy}), —S(O)ₙR^{z}, —N(R^{xx})S(O)ₙN(R^{xx})(R^{yy}), —S(O)ₙN(R^{xx})(R^{yy}), or —N(R^{x})S(O)ₙR^{z};
   each R^{x}, R^{xx}, R^{y}, R^{yy}, and R^{z} are independently H, (C₁-C₆)alkyl, (C₁-C₆)haloalkyl, or (C₁-C₆)cycloalkyl; and
   each n is independently 0-2.

In some embodiments of a compound of formula (I), or a stereoisomer or tautomer thereof, or a pharmaceutically acceptable salt of any of the foregoing, L is (C₁-C₂₀)alkyl, wherein the (C₁-C₂₀)alkyl is optionally substituted with one or more deutero, halo, (C₁-C₆)alkyl, (C₁-C₆)haloalkyl, oxo (C═O), —O(C₁-C₆)alkyl, —O(C₁-C₆)haloalkyl, —O(C₁-C₆)cycloalkyl, —N(R^{x})(R^{y})—, C₃-C₈ cycloalkyl, 3- to 8-membered heterocyclcyl, C₆-C₁₀ aryl, or 5- to 10-membered heteroaryl; and
one or more of the C atoms of the (C₁-C₂₀)alkyl is optionally replaced with one or more —C(R^{x})═C(R^{x})—, —C≡C—, —O—, —C(O)—, —N(R^{x})C(O)N(R^{y})—, —C(O)N(R^{x})—, —N(R^{x})C(O)—, —N(R^{x})—, —S(O)ₙ—, —N(R^{x})S(O)ₙN(R^{y})—, —S(O)ₙN(R^{x})—, —N(R^{x})S(O)ₙ—, C₃-C₈ cycloalkyl, 3- to 8-membered heterocyclcyl, C₆-C₁₀ aryl, or 5- to 10-membered heteroaryl;
wherein
   each C₃-C₈ cycloalkyl, 3- to 8-membered heterocyclcyl, C₆-C₁₀ aryl, and 5- to 10-membered heteroaryl are optionally substituted with one or more deutero, halo, (C₁-C₆)alkyl, (C₁-C₆)haloalkyl, oxo (C═O), —O(C₁-C₆)alkyl, —O(C₁-C₆)haloalkyl, —O(C₁-C₆)cycloalkyl, —N(R^{xx})(R^{yy}), —C(O)R^{z}, —C(O)OR^{z}, —OC(O)R^{z}, —N(R^{xx})C(O)N((R^{xx})(R^{yy}), —C(O)N(R^{xx})(R^{yy}), —N(R^{xx})(R^{yy}), —S(O)ₙR^{z}, —N(R^{xx})S(O)ₙN(R^{xx})(R^{yy}), —S(O)ₙN(R^{xx})(R^{yy}), or —N(R^{xx})S(O)ₙR^{z};
   each R^{x}, R^{xx}, R^{y}, R^{yy}, and R^{z} are independently H, (C₁-C₆)alkyl, (C₁-C₆)haloalkyl, or (C₁-C₆)cycloalkyl; and
   each n is independently 0-2.

In some embodiments of a compound of formula (I), or a stereoisomer or tautomer thereof, or a pharmaceutically acceptable salt of any of the foregoing, L is (C₁-C₂₀)alkyl, wherein the (C₁-C₂₀)alkyl is optionally substituted with one or more deutero, halo, (C₁-C₆)alkyl, (C₁-C₆)haloalkyl, oxo (C═O), —O(C₁-C₆)alkyl, —O(C₁-C₆)haloalkyl, —O(C₁-C₆)cycloalkyl, —N(R^{x})(R^{y})—, C₃-C₈ cycloalkyl, 3- to 8-membered heterocyclcyl, C₆-C₁₀ aryl, or 5- to 10-membered heteroaryl; and
one or more of the C atoms of the (C₁-C₂₀)alkyl is optionally replaced with one or more —C(R^{x})═C(R^{x})—, —C≡C—, —O—, —C(O)—, —N(R^{x})C(O)N(R^{y})—, —C(O)N(R^{x})—, —N(R^{x})C(O)—, —N(R^{x})—, —S(O)ₙ—, —N(R^{x})S(O)ₙN(R^{y})—, —S(O)ₙN(R^{x})—, —N(R^{x})S(O)ₙ—, C₃-C₈ cycloalkyl, 3- to 8-membered heterocyclcyl, C₆-C₁₀ aryl, or 5- to 10-membered heteroaryl;
wherein
   each R^{x} and R^{y} are independently H, (C₁-C₆)alkyl, (C₁-C₆)haloalkyl, or (C₁-C₆)cycloalkyl; and
   each n is independently 0-2.

In some embodiments of a compound of formula (I), or a stereoisomer or tautomer thereof, or a pharmaceutically acceptable salt of any of the foregoing, L is (C₁-C₂₀)alkyl, wherein the (C₁-C₂₀)alkyl is optionally substituted with one or more deutero, halo, (C₁-C₆)alkyl, (C₁-C₆)haloalkyl, oxo (C═O), —O(C₁-C₆)alkyl, —O(C₁-C₆)haloalkyl, —O(C₁-C₆)cycloalkyl, or —N(R^{x})(R^{y})—; and
one or more of the C atoms of the (C₁-C₂₀)alkyl is optionally replaced with one or more —O—, —C(O)—, —N(R^{x})C(O)N(R^{y})—, —C(O)N(R^{x})—, —N(R^{x})C(O)—, —N(R^{x})—, —S(O)ₙ—, —N(R^{x})S(O)ₙN(R^{y})—, —S(O)ₙN(R^{x})—, —N(R^{x})S(O)ₙ—, C₃-C₈ cycloalkyl, 3- to 8-membered heterocyclcyl, C₆-C₁₀ aryl, or 5- to 10-membered heteroaryl;
wherein
   each R^{x} and R^{y} are independently H, (C₁-C₆)alkyl, (C₁-C₆)haloalkyl, or (C₁-C₆)cycloalkyl; and
   each n is independently 0-2.

In some embodiments of a compound of formula (I), or a stereoisomer or tautomer thereof, or a pharmaceutically acceptable salt of any of the foregoing, L is (C₁-C₂₀)alkyl, wherein the (C₁-C₂₀)alkyl is optionally substituted with one or more deutero, halo, (C₁-C₄)alkyl, (C₁-C₄)haloalkyl, oxo (C═O), —O(C₁-C₄)alkyl, —O(C₁-C₄)haloalkyl, or —O(C₁-C₄)cycloalkyl; and
one or more of the C atoms of the (C₁-C₂₀)alkyl is optionally replaced with one or more —O—, —C(O)—, —N(R^{x})C(O)N(R^{y})—, —C(O)N(R^{x})—, —N(R^{x})C(O)—, —N(R^{x})—, 3- to 8-membered heterocyclcyl, C₆-C₁₀ aryl, or 5- to 10-membered heteroaryl;
wherein
   each R^{x} and R^{y} are independently H, (C₁-C₃)alkyl, (C₁-C₃)haloalkyl, or (C₁-C₃)cycloalkyl; and
   each n is independently 0-2.

In some embodiments of a compound of formula (I), or a stereoisomer or tautomer thereof, or a pharmaceutically acceptable salt of any of the foregoing, L is (C₁-C₂₀)alkyl, wherein one or more C atoms of the (C₁-C₂₀)alkyl is optionally replaced with one or more —O—, —C(O)—, —N(R^{x})C(O)N(R^{y})—, —C(O)N(R^{x})—, —N(R^{x})C(O)—, or —N(R^{x})—; wherein
each R^{x} and R^{y} are independently H, (C₁-C₃)alkyl, (C₁-C₃)haloalkyl, or (C₁-C₃)cycloalkyl; and
each n is independently 0-2.

In some embodiments of a compound of formula (I), or a stereoisomer or tautomer thereof, or a pharmaceutically acceptable salt of any of the foregoing, L is (C₁-C₂₀)alkyl, wherein wherein one or more of the C atoms of the (C₁-C₂₀)alkyl is replaced with one or more —O—.

In some embodiments of a compound of formula (I), or a stereoisomer or tautomer thereof, or a pharmaceutically acceptable salt of any of the foregoing, L is -(—O—CH₂CH₂)ₘ—O—, wherein m is 1-12.

In some embodiments of a compound of formula (I), or a stereoisomer or tautomer thereof, or a pharmaceutically acceptable salt of any of the foregoing, L is alkyl. In some embodiments, the alkyl is C₁-C₂, C₁-C₃, C₁-C₄, C₁-C₅, C₁-C₆, C₁-C₈, C₁-C₁₀, C₁-C₁₂, C₁-C₁₅, C₁-C₂₀, C₁-C₃₀, C₁-C₄₀, or C₁-C₅₀. In some embodiments, the alkyl is C₁-C₅₀. In some embodiments, the alkyl is C₁-C₄₀. In some embodiments, the alkyl is C₁-C₃₀. In some embodiments, the alkyl is C₁-C₂₀. In some embodiments, the alkyl is C₁-C₁₅. In some embodiments, the alkyl is C₁-C₁₂. In some embodiments, the alkyl is C₁-C₁₀. In some embodiments, the alkyl is C₁-C₈. In some embodiments, the alkyl is C₁-C₆. In some embodiments, the alkyl is C₁-C₄. In some embodiments, the alkyl is C₁-C₃. In some embodiments, the alkyl is methyl or ethyl. In some embodiments, the alkyl is methyl. In some embodiments, the alkyl is ethyl. In some embodiments, the alkyl is unbranched. In some embodiments, the alkyl is branched.

In some embodiments of a compound of formula (I), or a stereoisomer or tautomer thereof, or a pharmaceutically acceptable salt of any of the foregoing, the alkyl of L is optionally substituted with one or more deutero, halo, (C₁-C₆)alkyl, (C₁-C₆)haloalkyl, oxo (C═O), —O(C₁-C₆)alkyl, —O(C₁-C₆)haloalkyl, —O(C₁-C₆)cycloalkyl, —N(R^{x})(R^{y})—, C₃-C₁₅ carbocyclcyl, 3- to 15-membered heterocyclcyl, C₆-C₁₄ aryl, or 5- to 20-membered heteroaryl. In some embodiments, the alkyl is optionally substituted with one or more deutero, halo, (C₁-C₆)alkyl, (C₁-C₆)haloalkyl, oxo (C═O), —O(C₁-C₆)alkyl, —O(C₁-C₆)haloalkyl, —O(C₁-C₆)cycloalkyl, —N(R^{x})(R^{y})—, C₃-C₈ cycloalkyl, 3- to 8-membered heterocyclcyl, C₆-C₁₀ aryl, or 5- to 10-membered heteroaryl.

In some embodiments of a compound of formula (I), or a stereoisomer or tautomer thereof, or a pharmaceutically acceptable salt of any of the foregoing, the alkyl of L is optionally substituted with one or more deutero, halo, (C₁-C₆)alkyl, (C₁-C₆)haloalkyl, oxo (C═O), —O(C₁-C₆)alkyl, —O(C₁-C₆)haloalkyl, —O(C₁-C₆)cycloalkyl, or —N(R^{x})(R^{y})—. In some embodiments, the alkyl is optionally substituted with one or more deutero, halo, (C₁-C₄)alkyl, (C₁-C₄)haloalkyl, oxo (C═O), —O(C₁-C₄)alkyl, —O(C₁-C₄)haloalkyl, or —O(C₁-C₄)cycloalkyl.

In some embodiments of a compound of formula (I), or a stereoisomer or tautomer thereof, or a pharmaceutically acceptable salt of any of the foregoing, one or more of the C atoms of the alkyl of L is optionally replaced with one or more —C(R^{x})═C(R^{x})—, —C≡C—, —O—, —C(O)—, —C(O)O—, —OC(O)—, —OC(O)O—, —OC(O)N(R^{x})—, —N(R^{x})C(O)O—, —N(R^{x})C(O)N(R^{y})—, —C(O)N(R^{x})—, —N(R^{x})C(O)—, —N(R^{x})—, —S(O)ₙ—, —S(O)ₙO—, —OS(O)ₙ—, —OS(O)ₙO—, —OS(O)ₙN(R^{x})—, —N(R^{x})S(O)ₙO—, —N(R^{x})S(O)ₙN(R^{y})—, —S(O)ₙN(R^{x})—, —N(R^{x})S(O)ₙ—, C₃-C₁₅ carbocyclcyl, 3- to 15-membered heterocyclcyl, C₆-C₁₄ aryl, or 5- to 20-membered heteroaryl. In some embodiments, one or more of the C atoms of the alkyl is optionally replaced with one or more —C(R^{x})═C(R^{x})—, —C≡C—, —O—, —C(O)—, —N(R^{x})C(O)N(R^{y})—, —C(O)N(R^{x})—, —N(R^{x})C(O)—, —N(R^{x})—, —S(O)ₙ—, —N(R^{x})S(O)ₙN(R^{y})—, —S(O)ₙN(R^{x})—, —N(R^{x})S(O)ₙ—, C₃-C₁₅ carbocyclcyl, 3- to 15-membered heterocyclcyl, C₆-C₁₄ aryl, or 5- to 20-membered heteroaryl. In some embodiments, one or more of the C atoms of the alkyl is optionally replaced with one or more —C(R^{x})═C(R^{x})—, —C≡C—, —O—, —C(O)—, —N(R^{x})C(O)N(R^{y})—, —C(O)N(R^{x})—, —N(R^{x})C(O)—, —N(R^{x})—, —S(O)ₙ—, —N(R^{x})S(O)ₙN(R^{y})—, —S(O)ₙN(R^{x})—, —N(R^{x})S(O)ₙ—, C₃-C₈ cycloalkyl, 3- to 8-membered heterocyclcyl, C₆-C₁₀ aryl, or 5- to 10-membered heteroaryl. In some embodiments, one of more of the C atoms of the alkyl is optionally replaced with one or more —O—, —C(O)—, —N(R^{x})C(O)N(R^{y})—, —C(O)N(R)—, —N(R^{x})C(O)—, —N(R^{x})—, —S(O)ₙ—, —N(R^{x})S(O)ₙN(R^{y})—, —S(O)ₙN(R^{x})—, —N(R^{x})S(O)ₙ—, C₃-C₈ cycloalkyl, 3- to 8-membered heterocyclcyl, C₆-C₁₀ aryl, or 5- to 10-membered heteroaryl. In some embodiments, one or more of the C atoms of the alkyl is optionally replaced with one or more —O—, —C(O)—, —N(R^{x})C(O)N(R^{y})—, —C(O)N(R^{x})—, —N(R^{x})C(O)—, —N(R^{x})—, 3- to 8-membered heterocyclcyl, C₆-C₁₀ aryl, or 5- to 10-membered heteroaryl. In some embodiments, one or more of the C atoms of the alkyl is optionally replaced with one or more —O—, —C(O)—, —N(R^{x})C(O)N(R^{y})—, —C(O)N(R^{x})—, —N(R^{x})C(O)—, or —N(R^{x})—. In some embodiments, the one or more of the C atoms of the alkyl is optionally replaced with one or more —O— or —N(R^{x})—. In some embodiments, the one or more of the C atoms of the alkyl is optionally replaced with one or more —O—.

In some embodiments of a compound of formula (I), or a stereoisomer or tautomer thereof, or a pharmaceutically acceptable salt of any of the foregoing, each C₃-C₁₅ carbocyclcyl, 3- to 15-membered heterocyclcyl, C₆-C₁₄ aryl, or 5- to 20-membered heteroaryl of L is optionally substituted with one or more deutero, halo, (C₁-C₆)alkyl, (C₁-C₆)haloalkyl, oxo (C═O), —O(C₁-C₆)alkyl, —O(C₁-C₆)haloalkyl, —O(C₁-C₆)cycloalkyl, —N(R^{xx})(R^{yy}), —C(O)R^{z}, —C(O)OR^{z}, —OC(O)R^{z}, —OC(O)OR^{z}, —OC(O)N(R^{xx})(R^{yy}), —N(R^{xx})C(O)OR^{z}, —N(R^{xx})C(O)N((R^{xx})(Rⁿ), —C(O)N(R^{xx})(R^{yy}), —N(R^{xx})C(O)R^{z}, —N(R^{xx})(R^{yy}), —S(O)ₙR^{z}, —S(O)ₙOR^{z}, —OS(O)ₙR^{z}, —OS(O)ₙOR^{z}, —OS(O)ₙN(R^{xx})(R^{yy}), —N(R^{xx})S(O)ₙOR^{z}, —N(R^{xx})S(O)ₙN(R^{x})(R^{y}), —S(O)ₙN(R^{xx})(R^{yy}), or —N(R^{xx})S(O)ₙR^{z}; wherein each R^{x}, R^{xx}, R^{y}, R^{yy}, and R^{z} are independently H, (C₁-C₆)alkyl, (C₁-C₆)haloalkyl, or (C₁-C₆)cycloalkyl; and each n is independently 0-2.

In some embodiments of a compound of formula (I), or a stereoisomer or tautomer thereof, or a pharmaceutically acceptable salt of any of the foregoing, each C₃-C₁₅ carbocyclcyl, 3- to 15-membered heterocyclcyl, C₆-C₁₄ aryl, and 5- to 20-membered heteroaryl of L is optionally substituted with one or more deutero, halo, (C₁-C₆)alkyl, (C₁-C₆)haloalkyl, oxo (C═O), —O(C₁-C₆)alkyl, —O(C₁-C₆)haloalkyl, —O(C₁-C₆)cycloalkyl, —N(R^{xx})(R^{yy}), —C(O)R^{z}, —C(O)OR^{z}, —OC(O)R^{z}, —N(R^{xx})C(O)N((R^{x})(R^{y}), —C(O)N(R^{xx})(R^{yy}), —N(R^{xx})(R^{yy}), —S(O)ₙR^{z}, —N(R^{xx})S(O)ₙN(R^{xx})(R^{yy}), —S(O)ₙN(R^{xx})(R^{yy}), or —N(R^{xx})S(O)ₙR^{z}; wherein
each R^{x}, R^{xx}, R^{y}, R^{yy}, and R^{z} are independently H, (C₁-C₆)alkyl, (C₁-C₆)haloalkyl, or (C₁-C₆)cycloalkyl; and
each n is independently 0-2.

In some embodiments of a compound of formula (I), or a stereoisomer or tautomer thereof, or a pharmaceutically acceptable salt of any of the foregoing, each C₃-C₈ cycloalkyl, 3- to 8-membered heterocyclcyl, C₆-C₁₀ aryl, and 5- to 10-membered heteroaryl of L is optionally substituted with one or more deutero, halo, (C₁-C₆)alkyl, (C₁-C₆)haloalkyl, oxo (C═O), —O(C₁-C₆)alkyl, —O(C₁-C₆)haloalkyl, —O(C₁-C₆)cycloalkyl, —N(R^{xx})(Rⁿ), —C(O)R^{z}, —C(O)OR^{z}, —OC(O)R^{z}, —N(R^{xx})C(O)N((R^{xx})(R^{yy}), —C(O)N(R^{xx})(R^{yy}), —N(R^{xx})(R^{yy}), —S(O)ₙR^{z}, —N(R^{xx})S(O)ₙN(R^{xx})(R^{yy}), —S(O)ₙN(R^{xx})(R^{yy}), or —N(R^{xx})S(O)ₙR^{z}; wherein
each R^{x}, R^{xx}, R^{y}, R^{yy}, and R^{z} are independently H, (C₁-C₆)alkyl, (C₁-C₆)haloalkyl, or (C₁-C₆)cycloalkyl; and
each n is independently 0-2.

In some embodiments of a compound of formula (I), or a stereoisomer or tautomer thereof, or a pharmaceutically acceptable salt of any of the foregoing, L is -(—O—CH₂CH₂)ₘ—O—, wherein m is 1-25. In some embodiments, L is -(—O—CH₂CH₂)ₘ—O—, wherein m is 1-20. In some embodiments, L is -(—O—CH₂CH₂)ₘ—O—, wherein m is 1-15. In some embodiments, L is -(—O—CH₂CH₂)ₘ—O—, wherein m is 1-10. In some embodiments, L is -(—O—CH₂CH₂)ₘ—O—, wherein m is 1-6. In some embodiments, L is -(—O—CH₂CH₂)ₘ—O—, wherein m is 1-4. In some embodiments, L is -(—O—CH₂CH₂)ₘ—O—, wherein m is 1-3. In some embodiments, L is -(—O—CH₂CH₂)ₘ—O—, wherein m is 1-2. In some embodiments, L is -(—O—CH₂CH₂)—O—. In some embodiments, m is 1. In some embodiments, m is 2. In some embodiments, m is 3. In some embodiments, m is 4. In some embodiments, m is 5. In some embodiments, m is 6. In some embodiments, m is 10. In some embodiments, m is 11.

In some embodiments of a compound of formula (I), or a stereoisomer or tautomer thereof, or a pharmaceutically acceptable salt of any of the foregoing, L is -(—O—CH₂CH₂)ₘ—O—, wherein m is 1-25. In some embodiments, L is -(—O—CH₂CH₂)ₘ—O—, wherein m is 1-20. In some embodiments, L is -(—O—CH₂CH₂)ₘ—O—, wherein m is 1-15. In some embodiments, L is -(—O—CH₂CH₂)ₘ—O—, wherein m is 1-10. In some embodiments, L is -(—O—CH₂CH₂)ₘ—O—, wherein m is 1-6. In some embodiments, L is -(—O—CH₂CH₂)ₘ—O—, wherein m is 1-4. In some embodiments, L is -(—O—CH₂CH₂)ₘ—O—, wherein m is 1-3. In some embodiments, L is -(—O—CH₂CH₂)ₘ—O—, wherein m is 1-2. In some embodiments, L is -(—O—CH₂CH₂)—O—. In some embodiments, m is 1. In some embodiments, m is 2. In some embodiments, m is 3. In some embodiments, m is 4. In some embodiments, m is 5. In some embodiments, m is 6. In some embodiments, m is 10. In some embodiments, m is 11.

In some embodiments of a compound of formula (I), or a stereoisomer or tautomer thereof, or a pharmaceutically acceptable salt of any of the foregoing, L is -(—O—CH₂CH₂)ₘ—O—, wherein m is 1-25. In some embodiments, L is -(—O—CH₂CH₂)ₘ—O—, wherein m is 1-20, and one or more O is replaced with NH. In some embodiments, L is -(—O—CH₂CH₂)ₘ—O—, wherein m is 1-15, and one or more O is replaced with NH. In some embodiments, L is -(—O—CH₂CH₂)ₘ—O—, wherein m is 1-10, and one or more O is replaced with NH. In some embodiments, L is -(—O—CH₂CH₂)ₘ—O—, wherein m is 1-6, and one or more O is replaced with NH. In some embodiments, L is -(—O—CH₂CH₂)ₘ—O—, wherein m is 1-4, and one or more O is replaced with NH. In some embodiments, L is -(—O—CH₂CH₂)ₘ—O—, wherein m is 1-3, and one or more O is replaced with NH. In some embodiments, L is -(—O—CH₂CH₂)ₘ—O—, wherein m is 1-2, and one or more O is replaced with NH. In some embodiments, L is -(—O—CH₂CH₂)—O—. In some embodiments, m is 1, and one O is replaced with NH. In some embodiments, m is 2, and one O is replaced with NH. In some embodiments, m is 3, and one O is replaced with NH. In some embodiments, m is 4, and one O is replaced with NH. In some embodiments, m is 5. In some embodiments, m is 6, and one O is replaced with NH. In some embodiments, m is 10, and one O is replaced with NH. In some embodiments, m is 11, and one O is replaced with NH. In some embodiments, L is —O—CH₂CH₂—O—CH₂CH₂—O—CH₂CH₂—NH—CH₂CH₂—O—CH₂CH₂—O—.

In some embodiments of a compound of formula (I), or a stereoisomer or tautomer thereof, or a pharmaceutically acceptable salt of any of the foregoing, L comprises an optionally substituted alkylene, optionally substituted heteroalkylene, optionally substituted alkenylene, optionally substituted heteroalkenylene, optionally substituted alkynylene, optionally substituted heteroalkynylene, optionally substituted carbocyclylene, optionally substituted heterocyclylene, optionally substituted arylene, optionally substituted heteroarylene, or any combination thereof.

In some embodiments of a compound of formula (I), or a stereoisomer or tautomer thereof, or a pharmaceutically acceptable salt of any of the foregoing, L is selected from the group consisting of ethylene glycol, polyethylene glycol (PEG), and PEG derivatives. In some embodiments a PEG derivative comprises a polyethylene glycol (PEG), wherein one or more of the C atoms of the PEG is substituted with one or more groups, and/or one or more of the C atoms of the PEG is replaced with one or more groups. In some embodiments the one or more C atoms in PEG are each independently substituted with one or more deutero, halo, (C₁-C₆)alkyl, (C₁-C₆)haloalkyl, oxo (C═O), —O(C₁-C₆)alkyl, —O(C₁-C₆)haloalkyl, —O(C₁-C₆)cycloalkyl, —N(R^{x})(R^{y})—, C₃-C₁₅ carbocyclcyl, 3- to 15-membered heterocyclcyl, C₆-C₁₄ aryl, or 5- to 20-membered heteroaryl; and/or the one or more of the C atoms in the PEG moiety are each independently replaced with one or more —C(R^{x})═C(R^{x})—, —C≡C—, —O—, —C(O)—, —C(O)O—, —OC(O)—, —OC(O)O—, —OC(O)N(R^{x})—, —N(R^{x})C(O)O—, —N(R^{x})C(O)N(R^{y})—, —C(O)N(R^{x})—, —N(R^{x})C(O)—, —N(R^{x})—, —S(O)ₙ—, —S(O)ₙO—, —OS(O)ₙ—, —OS(O)ₙO—, —OS(O)ₙN(R^{x})—, —N(R^{x})S(O)ₙO—, —N(R^{x})S(O)ₙN(R^{y})—, —S(O)ₙN(R^{x})—, —N(R^{x})S(O)ₙ—, C₃-C₁₅ carbocyclcyl, 3- to 15-membered heterocyclcyl, C₆-C₁₄ aryl, or 5- to 20-membered heteroaryl;

wherein
each C₃-C₁₅ carbocyclcyl, 3- to 15-membered heterocyclcyl, C₆-C₁₄ aryl, or 5- to 20-membered heteroaryl is optionally substituted with one or more deutero, halo, (C₁-C₆)alkyl, (C₁-C₆)haloalkyl, oxo (C═O), —O(C₁-C₆)alkyl, —O(C₁-C₆)haloalkyl, —O(C₁-C₆)cycloalkyl, —N(R^{xx})(R^{yy}), —C(O)R^{z}, —C(O)OR^{z}, —OC(O)R^{z}, —OC(O)OR^{z}, —OC(O)N(R^{xx})(R^{yy}), —N(R^{xx})C(O)OR^{z}, —N(R^{x})C(O)N((R^{x})(R^{y}), —C(O)N(R^{xx})(R^{yy}), —N(R^{xx})C(O)R^{z}, —N(R^{xx})(R^{yy}), —S(O)ₙR^{z}, —S(O)ₙOR^{z}, —OS(O)ₙR^{z}, —OS(O)ₙOR^{z}, —OS(O)ₙN(R^{x})(R^{y}Y), —N(R^{xx})S(O)ₙOR^{z}, —N(R^{xx})S(O)ₙN(R^{xx})(R^{y}), —S(O)ₙN(R^{xx})(R^{yy}), or —N(R′)S(O)ₙR^{z};
each R^{x}, R^{xx}, R^{y}, R^{yy}, and R^{z} are independently H, (C₁-C₆)alkyl, (C₁-C₆)haloalkyl, or (C₁-C₆)cycloalkyl; and
each n is independently 0-2.

In some embodiments of a compound of formula (I), or a stereoisomer or tautomer thereof, or a pharmaceutically acceptable salt of any of the foregoing, L comprises a polymer.

In some embodiments of a compound of formula (I), or a stereoisomer or tautomer thereof, or a pharmaceutically acceptable salt of any of the foregoing, L comprises a polymer. In some embodiments, the polymer is polyethylene glycol (PEG). In some embodiments, the polyethylene glycol comprises from about 2 to about 20 ethylene glycol units.

In some embodiments of a compound of formula (I), or a stereoisomer or tautomer thereof, or a pharmaceutically acceptable salt of any of the foregoing, L is a bond.

In some embodiments of a compound of formula (I), or a stereoisomer or tautomer thereof, or a pharmaceutically acceptable salt of any of the foregoing, L is bound at any available position of G1 or Z1, and any available position of G2 or Z2.

In some embodiments of a compound of formula (I), or a stereoisomer or tautomer thereof, or a pharmaceutically acceptable salt of any of the foregoing, L is bound to:
(i) the Z1 moiety of (G1-Z1); and
(ii) the Z2 moiety of (G2-Z2).

In some embodiments of a compound of formula (I), or a stereoisomer or tautomer thereof, or a pharmaceutically acceptable salt of any of the foregoing, L is bound to:
(i) the G1 moiety of (G1-Z1); and
(ii) the G2 moiety of (G2-Z2).

In some embodiments of a compound of formula (I), or a stereoisomer or tautomer thereof, or a pharmaceutically acceptable salt of any of the foregoing, L is bound to:
(i) the Z1 moiety of (G1-Z1); and
(ii) the G2 moiety of (G2-Z2).

In some embodiments of a compound of formula (I), or a stereoisomer or tautomer thereof, or a pharmaceutically acceptable salt of any of the foregoing, L is bound to:
(i) the G1 moiety of (G1-Z1); and
(ii) the Z2 moiety of (G2-Z2).

In some embodiments of a compound of formula (I-1), or (I-2), or a stereoisomer or tautomer thereof, or a pharmaceutically acceptable salt of any of the foregoing, when R² is unsubstituted methyl, then either:
(1) Q¹ is 5-20 membered heteroaryl, wherein the 5-20 membered heteroaryl of Q¹ is optionally substituted with one or more halo, C₁₋₆alkyl, C₂₋₆alkenyl, C₁₋₆alkoxy, —NH₂, C₃₋₁₀cycloalkyl, or —OH, and wherein Q¹ is not unsubstituted pyridyl, or
(2) Q¹ is phenyl, wherein the phenyl of Q¹ is substituted with
   (i) at least one C₃₋₆alkyl, wherein the at least one C₃₋₆alkyl is optionally substituted with one or more halo, or
   (ii) at least one C₃₋₁₀cycloalkyl, wherein the at least one C₃₋₁₀cycloalkyl is optionally substituted with one or more halo or C₁₋₆alkyl, or
   (iii) at least one 5-20 membered heteroaryl, wherein the at least one 5-20 membered heteroaryl is optionally substituted with one or more C₁₋₆alkyl.

In some embodiments of a compound of formula (I), or a stereoisomer or tautomer thereof, or a pharmaceutically acceptable salt of any of the foregoing, (i) G1 or G1-Z1, and (ii) G2 or G2-Z2 are identical. In some embodiments, G1 and G2 are identical and/or G1-Z1 and G2-Z2 are identical. In some embodiments, (i) G1 and (ii) G2 are identical. In some embodiments, (i) G1-Z1, and (ii) G2-Z2 are identical.

In some embodiments of a compound of formula (I), or a stereoisomer or tautomer thereof, or a pharmaceutically acceptable salt of any of the foregoing, (i) G1 or G1-Z1, and (ii) G2 or G2-Z2 are different. In some embodiments, G1 and G2 are identical and/or G1-Z1 and G2-Z2 are different. In some embodiments, (i) G1 and (ii) G2 are different. In some embodiments, (i) G1-Z1, and (ii) G2-Z2 are different.

In some embodiments of a compound of formula (I), or a stereoisomer or tautomer thereof, or a pharmaceutically acceptable salt of any of the foregoing, (i) G1 or G1-Z1, and (ii) G2 or G2-Z2, has enhanced GYS1 inhibition compared to the other of (i) G1 or G1-Z1, and (ii) G2 or G2-Z2. In some embodiments, one of G1 and G2 has enhanced GYS1 inhibition compared to the other of G1 and G2. In some embodiments, one of G1-Z1 and G2-Z2 has enhanced GYS1 inhibition compared to the other of G1-Z1 and G2-Z2.

In some embodiments of a compound of formula (I), or a stereoisomer or tautomer thereof, or a pharmaceutically acceptable salt of any of the foregoing, the compound of formula (I) has enhanced GYS1 inhibition compared to G1, G1-Z1, G2, and/or G2-Z2. In some embodiments, the compound of formula (I) has enhanced GYS1 inhibition compared to G1. In some embodiments, the compound of formula (I) has enhanced GYS1 inhibition compared to G1-Z1. In some embodiments, the compound of formula (I) has enhanced GYS1 inhibition compared to G2. In some embodiments, the compound of formula (I) has enhanced GYS1 inhibition compared to G2-Z2.

In some embodiments, (G1-Z1)-L-(G2-Z2) is a bivalent inhibitor with the potential to simultaneously engage two GYS1 monomers within a single tetrameric complex.

In some embodiments of a compound of formula (I), or a stereoisomer or tautomer thereof, or a pharmaceutically acceptable salt of any of the foregoing, (G1-Z1) and (G2-Z2) are each, independently, of formula (I-1) or (I-2):

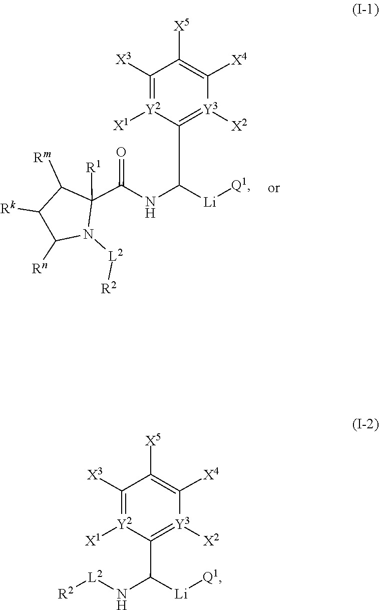

or a stereoisomer or tautomer thereof, or a pharmaceutically acceptable salt of any of the foregoing, wherein:
Y² and Y³ are each C, or
one of Y² and Y³ is N and the other of Y² and Y³ is C;
X¹ and X² are each independently H, C₁₋₆alkyl, or C₁₋₆alkoxy;
X³ and X⁴ are each independently H, halo, C₁₋₆alkyl, C₁₋₆alkoxy, or 5-20 membered heteroaryl, wherein the C₁₋₆alkyl of X³ and X⁴ is optionally substituted with one of more halo;
X⁵ is H, C₁₋₆alkyl, C₁₋₆alkoxy, or C₃₋₁₀cycloalkyl;
either
(1) L¹ is absent; and
Q¹ is selected from (i) to (iv):
(i) phenyl, wherein the phenyl of Q¹ is substituted with one or more halo, C₁₋₆alkyl, C₂₋₆alkenyl, —NH₂, —NH—C(O)—(C₁₋₆alkyl), —NH—C(O)-(3-15 membered heterocyclyl), C₃₋₁₀cycloalkyl, or 5-20 membered heteroaryl, wherein
   the C₁₋₆alkyl is optionally substituted with one or more halo, —NH—C(O)—NH(C₁₋₆alkyl), —NH—C(O)—C₁₋₆alkyl, or —NH—C(O)—C₁₋₆alkoxy,
   the C₃₋₁₀cycloalkyl is optionally substituted with one or more halo or C₁₋₆alkyl, and
   the 5-20 membered heteroaryl is optionally substituted with one or more C₁₋₆alkyl,
(ii) 3-15 membered heterocyclyl, wherein the 3-15 membered heterocyclyl of Q¹ is optionally substituted with one or more oxo, or C₁₋₆alkyl,
(iii) 5-20 membered heteroaryl, wherein the 5-20 membered heteroaryl of Q¹ is optionally substituted with one or more halo, C₁₋₆alkyl, C₂₋₆alkenyl, C₁₋₆alkoxy, —NH₂, or C₃₋₁₀cycloalkyl, wherein,
   the C₁₋₆alkyl is optionally substituted with one or more halo, and
   the C₃₋₁₀cycloalkyl is optionally substituted with one or more halo or C₁₋₆alkyl, and
(iv) C₃₋₁₀cycloalkyl;
or
(2) L¹ is —CH₂—; and
Q¹ is C₃₋₁₀cycloalkyl;
L² is —C(O)— or —S(O)₂—
R¹ is H or C₁₋₆alkyl;
R^{k} is H, halo, —OH, —NH₂, or —NH—C(O)C₁₋₆alkyl;
R^{m} is H, —OH, or C₁₋₆alkyl;
Rⁿ is H, C₁₋₆alkyl, or C₃₋₁₀cycloalkyl or Rⁿ taken together with the carbon atom to which it is attached forms C₃₋₅ cycloalkyl;
or R^{k} is taken together with either R^{m} or Rⁿ, and the atoms to which they are attached, to form cyclopropyl; and
R² is selected from (i) to (vii):
(i) C₁₋₆alkyl, wherein the C₁₋₆alkyl of R² is optionally substituted with one or more R^{a}, wherein R^{a} is:
   (a) —OH,
   (b) cyano,
   (c) C₂₋₆alkynyl,
   (d) C₆₋₂₀aryl, wherein the C₆₋₂₀aryl of R^{a} is optionally substituted with one or more halo, cyano, C₁₋₆alkoxy, or —NH—C(O)—C₁₋₆alkyl,
   (e) 3-15 membered heterocyclyl, wherein the 3-15 membered heterocyclyl of R^{a} is optionally substituted with one or more R^{c}, wherein
      R^{c} is halo, oxo, C₁₋₆alkyl, C₁₋₆alkoxy, —C(O)—C₁₋₆alkyl, or —C(O)—C₁₋₆alkoxy, wherein
         the C₁₋₆alkyl of R^{c} is optionally substituted with one or more halo or C₂₋₆alkynyl, and
         the —C(O)—C₁₋₆alkoxy of R^{c} is optionally substituted with one or more halo,
   (f) —N(R^{c})(R^{d}), wherein R^{c} and R^{d} of —N(R^{c})(R^{d}) are, independently of each other, H, C₁₋₆alkyl, —C(O)—C₁₋₆alkyl, —C(O)—C₁₋₆alkoxy, —C(O)—NH₂, —C(O)—NH(C₁₋₆alkyl), —C(O)—N(C₁₋₆alkyl)₂, —C(O)-(3-15 membered heterocyclyl), —CH₂—C(O)—NH₂, 3-15 membered heterocyclyl, or 5-20 membered heteroaryl, wherein
      the C₁₋₆alkyl of R^{c} or R^{d} is optionally substituted with one or more —C(O)—NH₂,
      the —C(O)—C₁₋₆alkyl of R^{c} or R^{d} is optionally substituted with one or more halo,
      the 3-15 membered heterocyclyl and the 5-20 membered heteroaryl of R^{c} or R^{d} are independently optionally substituted with one or more C₁₋₆alkyl,
      the —C(O)-(3-15 membered heterocyclyl) of R^{c} or R^{d} is optionally substituted with one or more halo, —C(O)—C₁₋₆alkoxy, or C₁₋₆alkyl, wherein the C₁₋₆alkyl is optionally substituted with one or more halo, C₁₋₆alkoxy, or C₃₋₁₀cycloalkyl, and
      the C₁₋₆alkyl of the —C(O)—N(C₁₋₆alkyl)₂ of R^{c} or R^{d} are, independently of each other, optionally substituted with one or more halo or C₆₋₂₀aryl,
   (g) —O—R^{e}, wherein R^{e} is C₁₋₆alkyl, C₆₋₂₀aryl, —C(O)-(3-15 membered heterocyclyl), —C(O)—N—(C₁₋₆alkyl)₂, or 5-20 membered heteroaryl, wherein
      the C₁₋₆alkyl of R^{e} is optionally substituted with one or more C₁₋₆alkoxy, wherein the C₁₋₆alkoxy is optionally substituted with one or more C₂₋₆alkynyl,
      the C₆₋₂₀aryl of R^{e} is optionally substituted with one or more C₁₋₆alkyl, and
      the —C(O)-(3-15 membered heterocyclyl) of R^{e} is optionally substituted with one or more C₁₋₆alkyl, C₁₋₆alkoxy, or —C(O)—C₁₋₆alkoxy, wherein the C₁₋₆alkyl is optionally substituted with one or more halo, C₁₋₆alkoxy, or C₃₋₁₀cycloalkyl,
   (h) —C(O)—R^{e}, wherein R^{e} is —NH₂, —OH, or 3-15 membered heterocyclyl, or
   (i) —S(O)₂—R^{f}, wherein R^{f} is C₁₋₆alkyl or 3-15 membered heterocyclyl,
(ii) C₃₋₁₀cycloalkyl, wherein the C₃₋₁₀cycloalkyl of R² is optionally substituted with one or more R^{q}, wherein R^{q} is 5-20 membered heteroaryl or C₆₋₂₀aryl, wherein the C₆₋₂₀aryl of R^{q} is optionally substituted with one or more C₁₋₆alkoxy,
(iii) 3-15 membered heterocyclyl, wherein the 3-15 membered heterocyclyl of R² is optionally substituted with one or more halo, oxo, C₁₋₆alkyl, —C(O)—C₁₋₆alkyl, or 5-20 membered heteroaryl,
(iv) 5-20 membered heteroaryl or —(C₁₋₄alkyl)(5-20 membered heteroaryl), wherein the C₁₋₄ alkyl is optionally substituted with one or more or more —OH, halo, —NH₂, —NH(C₁₋₆alkyl), —N(C₁₋₆alkyl)₂, and wherein the 5-20 membered heteroaryl of the 5-20 membered heteroaryl or —(C₁₋₄alkyl)(5-20 membered heteroaryl), is optionally substituted with one or more R^{s}, wherein
   R^{s} is halo, C₁₋₆alkyl, C₁₋₆alkoxy, —NH₂, —NH(C₁₋₆alkyl), —N(C₁₋₆alkyl)₂, —NH—C(O)—C₁₋₆alkyl, C₆₋₂₀aryl, C₃₋₁₀cycloalkyl, 3-15 membered heterocyclyl, 5-20 membered heteroaryl, or —C(O)—C₁₋₆alkoxy, wherein
      the C₁₋₆alkyl of R^{s} is optionally substituted with one or more halo, C₁₋₆alkoxy, —NH₂, —NH(C₁₋₆alkyl), —N(C₁₋₆alkyl)₂, —NH—C(O)C₁₋₆alkyl, or —NH—C(O)—C₁₋₆alkoxy, and
      the 3-15-membered heterocyclyl of R^{s} is optionally substituted with one or more halo or —C(O)—C₁₋₆alkoxy,
(v) —N(R^{g})(R^{h}), wherein R^{g} and R^{h} are independently H or C₁₋₆alkyl,
(vi) —C(O)—R^{j}, wherein R^{j} is C₃₋₁₀cycloalkyl, —NH(C₁₋₆alkyl), —N(C₁₋₆alkyl)₂, or —NH(5-20 membered heteroaryl), and
(vii) C₆₋₂₀aryl, wherein the C₆₋₂₀aryl of R² is optionally substituted with one or more 5-20 membered heteroaryl or —O—R^{p}, wherein R^{p} is 3-15 membered heterocyclyl, wherein the 3-15 membered heterocyclyl of R^{p} is optionally substituted with one or more —C(O)—C₁₋₆alkyl; and
   wherein (G1-Z1) and (Z2-G2) are each independently substituted with a group bound to L.

In some embodiments of a compound of formula (I), (I-1), or (I-2), or a stereoisomer or tautomer thereof, or a pharmaceutically acceptable salt of any of the foregoing, the group bound to L comprises a carbon, oxygen, nitrogen, or sulfur atom. In some embodiments, the group bound to L comprises a carbon atom. In some embodiments, the group bound to L comprises an oxygen atom. In some embodiments, the group bound to L comprises a nitrogen atom. In some embodiments, the group bound to L comprises a sulfur atom.

In some embodiments of a compound of formula (I), (I-1), or (I-2), or a stereoisomer or tautomer thereof, or a pharmaceutically acceptable salt of any of the foregoing, the group bound to L is at any available position of G1 or Z1, and any available position of G2 or Z2. In some embodiments, the group bound to L is at R² or Rⁿ. In some embodiments, the group bound to L is at R². In some embodiments, the group bound to L is at Rⁿ.

In some embodiments of a compound of formula (I), (I-1), or (I-2), or a stereoisomer or tautomer thereof, or a pharmaceutically acceptable salt of any of the foregoing the group bound to L comprises a reactive site suitable for binding to L. In some embodiments, the group bound to L comprises sulfhydryl groups to form disulfide bonds or thioether bonds, aldehyde, ketone, and hydrazine groups to form hydrazone bonds, carboxylic and amino groups to form peptide bonds, carboxylic and hydroxy groups to form ester bonds, sulfonic acids to form sulfonamide bonds, alcohols to form carbamate bonds, and amines to form amide bonds, sulfonamide bonds or carbamate bonds.

In some embodiments of a compound of formula (I), (I-1), or (I-2), such as a compound of formula (II), (III), (IV), or (V), or a stereoisomer or tautomer thereof, or a pharmaceutically acceptable salt of any of the foregoing, G1 and G2 are each, independently, the moiety of formula (I-1) represented by

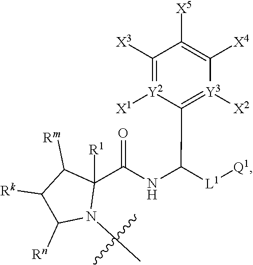

or the moiety of formula (I-1) or (I-2) represented by

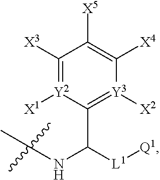

or a stereoisomer or tautomer thereof, or a pharmaceutically acceptable salt of any of the foregoing, wherein R^{k}, R^{m}, Rⁿ, R¹, X¹, X², X³, X⁴, X⁵, Y², Y³, L¹, and Q¹ are as defined for a compound of formula (I-1), or (I-2). In some embodiments, R^{k}, R^{m}, Rⁿ, R¹, X¹, X², X³, X⁴, X⁵, Y², Y³, L¹, and Q¹ are as defined for a compound of formula (I), (I-A), (I-A1), (I-A2), (I-A3), (I-A4), (I-B), (I-B1), (I-B2), (I-C), (I-D), (I-D1), (I-D2), (I-E), (I-F), (I-G), or (I-H) defined elsewhere herein.

In some embodiments of a compound of formula (I), (I-1), or (I-2), such as a compound of formula (II), (III), (IV), or (V), or a stereoisomer or tautomer thereof, or a pharmaceutically acceptable salt of any of the foregoing, G1 and G2 are each, independently, the moiety of formula (I-1) represented by

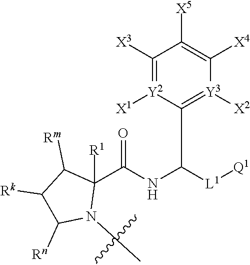

or a stereoisomer or tautomer thereof, or a pharmaceutically acceptable salt of any of the foregoing. In some embodiments, G1 and G2 are each, independently, the moiety of formula (I-2) represented by

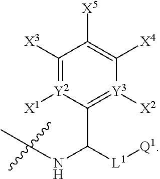

In some embodiments of a compound of formula (I), (I-1) or (I-2), or a stereoisomer or tautomer thereof, or a pharmaceutically acceptable salt of any of the foregoing, Z1 and Z2, are each, independently, the moiety of formula (I-1) or (I-2) represented by

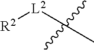

wherein R² and L² are as defined for a compound of formula (I-1), or (I-2). In some embodiments, R² and L² are as defined for a compound of formula (I), (I-A), (I-A1), (I-A2), (I-A3), (I-A4), (I-B), (I-B1), (I-B2), (I-C), (I-D), (I-D1), (I-D2), (I-E), (I-F), (I-G), or (I-H) defined elsewhere herein.

In some embodiments of a compound of formula (I-1) or (I-2), or a stereoisomer or tautomer thereof, or a pharmaceutically acceptable salt of any of the foregoing, Z1 and Z2, are each, independently, the moiety of formula (I-1) or (I-2) represented by

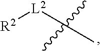

wherein L² is —C(O)— or —S(O)₂—, and R² is C₁₋₃alkyl, wherein the C₁₋₃alkyl or R² is optionally substituted with one or more 3-10 membered heterocycle, and wherein the 3-10 membered heterocycle is further optionally substituted with one or more oxo, or C₁₋₃alkyl. In some embodiments, Z1 and Z2, are each, independently, the moiety of formula (I-1) or (I-2) represented by

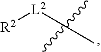

wherein L² is —C(O)—, and R² is C₁₋₃alkyl, wherein the C₁₋₃alkyl or R² is optionally substituted with one or more 3-10 membered heterocycle, and wherein the 3-10 membered heterocycle is further optionally substituted with one or more oxo, or C₁₋₃alkyl. In some embodiments, Z1 and Z2, are each, independently, the moiety of formula (I-1) or (I-2) represented by

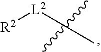

wherein L² is —S(O)₂—, and R² is C₁₋₃alkyl, wherein the C₁₋₃alkyl or R² is optionally substituted with one or more 3-10 membered heterocycle, and wherein the 3-10 membered heterocycle is further optionally substituted with one or more oxo, or C₁₋₃alkyl.

In some embodiments of a compound of formula (I-1) or (I-2), or a stereoisomer or tautomer thereof, or a pharmaceutically acceptable salt of any of the foregoing, Z1 and Z2, are each, independently, the moiety of formula (I-1) or (I-2) represented by

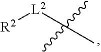

wherein L² is —C(O)— or —S(O)₂—, and R² is C₁₋₃alkyl. In some embodiments, Z1 and Z2, are each, independently, the moiety of formula (I-1) or (I-2) represented by

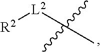

wherein L² is —C(O)—, and R² is C₁₋₃alkyl. In some embodiments, Z1 and Z2, are each, independently, the moiety of formula (I-1) or (I-2) represented by

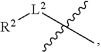

wherein L² is —S(O)₂—, and R² is C₁₋₃alkyl.

In some embodiments of a compound of formula (I-1) or (I-2), or a stereoisomer or tautomer thereof, or a pharmaceutically acceptable salt of any of the foregoing, Z1 and Z2, are each, independently, the moiety of formula (I-1) or (I-2) represented by

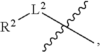

wherein L² is —C(O)— or —S(O)₂—. In some embodiments, Z1 and Z2, are each, independently, the moiety of formula (I-1) or (I-2) represented by

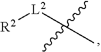

wherein L² is —C(O)—. In some embodiments, Z1 and Z2, are each, independently, the moiety of formula (I-1) or (I-2) represented by

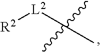

wherein L² is —S(O)₂—.

In some embodiments of a compound of formula (I-1) or (I-2), or a stereoisomer or tautomer thereof, or a pharmaceutically acceptable salt of any of the foregoing, L² is —C(O)— or —S(O)₂—. In some embodiments, L² is —C(O)—. In some embodiments, L² is —S(O)₂—.

In some embodiments of a compound of formula (I-1), or (I-2), or a stereoisomer or tautomer thereof, or a pharmaceutically acceptable salt of any of the foregoing, Q¹ is 5-20 membered heteroaryl, wherein the 5-20 membered heteroaryl of Q¹ is optionally substituted with one or more halo, C₁₋₆alkyl, C₂₋₆alkenyl, C₁₋₆alkoxy, —NH₂, or C₃₋₁₀cycloalkyl, wherein the C₁₋₆alkyl is optionally substituted with one or more halo, and the C₃₋₁₀cycloalkyl is optionally substituted with one or more C₁₋₆alkyl or halo. In some embodiments, Q¹ is 5-6 membered heteroaryl, wherein the 5-6 membered heteroaryl of Q¹ is optionally substituted with one or more halo, C₁₋₆alkyl, C₂₋₆alkenyl, C₁₋₆alkoxy, —NH₂, or C₃₋₁₀cycloalkyl, wherein the C₁₋₆alkyl is optionally substituted with one or more halo, and the C₃₋₁₀cycloalkyl is optionally substituted with one or more C₁₋₆alkyl or halo. In some embodiments, Q¹ is pyridinyl, wherein the pyridinyl of Q¹ is optionally substituted with one or more halo, C₁₋₆alkyl, C₂₋₆alkenyl, C₁₋₆alkoxy, —NH₂, or C₃₋₁₀cycloalkyl, wherein the C₁₋₆alkyl is optionally substituted with one or more halo, and the C₃₋₁₀cycloalkyl is optionally substituted with one or more C₁₋₆alkyl or halo. In some embodiments, Q¹ is 2-pyridinyl or 3-pyridinyl, wherein the 2-pyridinyl or 3-pyridinyl of Q¹ is optionally substituted with one or more halo, C₁₋₆alkyl, C₂₋₆alkenyl, C₁₋₆alkoxy, —NH₂, or C₃₋₁₀cycloalkyl, wherein the C₁₋₆alkyl is optionally substituted with one or more halo, and the C₃₋₁₀cycloalkyl is optionally substituted with one or more C₁₋₆alkyl or halo. In some embodiments, Q¹ is 2-pyridinyl, wherein the 2-pyridinyl of Q¹ is optionally substituted with one or more halo, C₁₋₆alkyl, C₁₋₆alkoxy, —NH₂, or C₃₋₁₀cycloalkyl, wherein the C₁₋₆alkyl is optionally substituted with one or more halo, and the C₃₋₁₀cycloalkyl is optionally substituted with one or more C₁₋₆alkyl or halo. In some embodiments Q¹ is 2-pyridinyl, wherein the 2-pyridinyl of Q¹ is optionally substituted with one or more halo, C₁₋₆alkyl, C₁₋₆alkoxy, or C₃₋₁₀cycloalkyl, wherein the C₁₋₆alkyl is optionally substituted with one or more halo, and the C₃₋₁₀cycloalkyl is optionally substituted with one or more C₁₋₆alkyl or halo. In some embodiments, Q¹ is 2-pyridinyl, wherein the 2-pyridinyl of Q¹ is optionally substituted with one or more fluoro, chloro, methyl, iso-propyl, tert-butyl, cyclopropyl, cyclobutyl, or methoxy, wherein the methyl is optionally substituted with one or more fluoro and the cyclopropyl and cyclobutyl are independently optionally substituted with one or more methyl or fluoro.

In some embodiments of a compound of formula (I-1), or (I-2), or a stereoisomer or tautomer thereof, or a pharmaceutically acceptable salt of any of the foregoing, Q¹ is selected

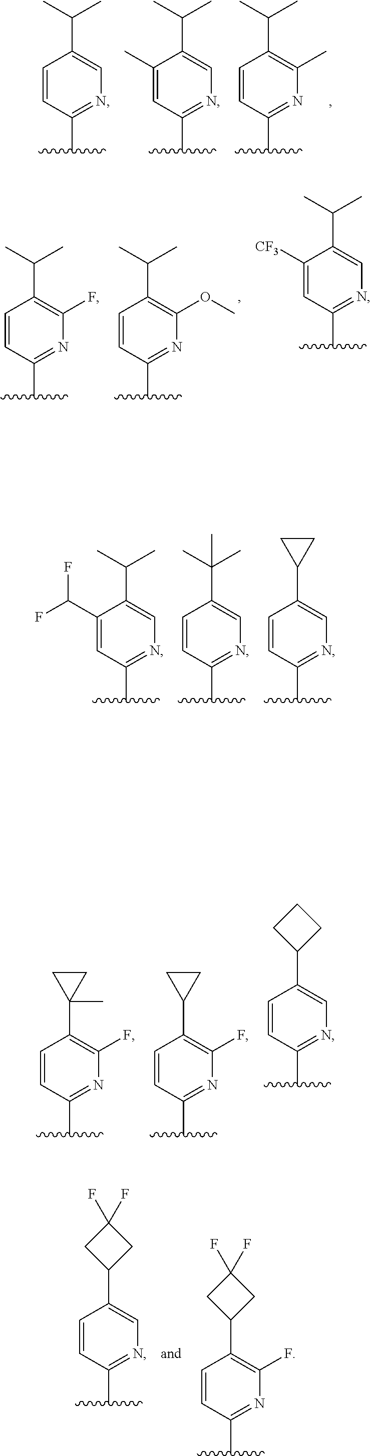

In some embodiments of a compound of formula (I-1), or (I-2), or a stereoisomer or tautomer thereof, or a pharmaceutically acceptable salt of any of the foregoing, Q¹ is selected from the group consisting of

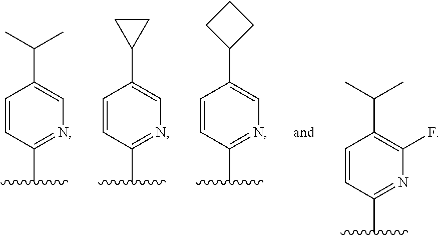

In some embodiments of a compound of formula (I-1), or (I-2), or a stereoisomer or tautomer thereof, or a pharmaceutically acceptable salt of any of the foregoing, Q¹ is phenyl, wherein the phenyl of Q¹ is substituted with one or more halo, C₁₋₆alkyl, C₂₋₆ alkenyl, —NH₂, —NH—C(O)—(C₁₋₆alkyl), —NH—C(O)-(3-15 membered heterocyclyl), or C₃₋₁₀cycloalkyl, wherein the C₁₋₆alkyl is optionally substituted with one or more halo, and the C₃₋₁₀cycloalkyl is optionally substituted with one or more halo or C₁₋₆alkyl. In some embodiments, Q¹ is phenyl, wherein the phenyl of Q¹ is substituted with one or more halo, C₁₋₆alkyl, C₂₋₆ alkenyl, or C₃₋₁₀cycloalkyl, wherein the C₁₋₆alkyl is optionally substituted with one or more halo, and the C₃₋₁₀cycloalkyl is optionally substituted with one or more halo or C₁₋₆alkyl. In some embodiments, Q¹ is phenyl, wherein the phenyl of Q¹ is substituted with one or more fluoro, chloro, methyl, iso-propyl, sec-butyl, tert-butyl, prop-1-en-2-yl, cyclopropyl, or cyclobutyl, wherein the methyl, iso-propyl, sec-butyl, and tert-butyl are independently optionally substituted with one or more halo, and the cyclopropyl and cyclobutyl are independently optionally substituted with one or more fluoro or methyl.

In some embodiments of a compound of formula (I-1), or (I-2), or a stereoisomer or tautomer thereof, or a pharmaceutically acceptable salt of any of the foregoing, Q¹ is phenyl, wherein the phenyl of Q¹ is substituted with one or more halo, C₁₋₆alkyl, C₂₋₆alkenyl, —NH₂, —NH—C(O)—(C₁₋₆alkyl), —NH—C(O)-(3-15 membered heterocyclyl), C₃₋₁₀cycloalkyl, or 5-20 membered heteroaryl, wherein the C₁₋₆alkyl is optionally substituted with one or more halo, —NH—C(O)—NH(C₁₋₆alkyl), —NH—C(O)—C₁₋₆alkyl, or —NH—C(O)—C₁₋₆alkoxy, the C₃₋₁₀cycloalkyl is optionally substituted with one or more halo or C₁₋₆alkyl, and the 5-20 membered heteroaryl is optionally substituted with one or more C₁₋₆alkyl.

In some embodiments of a compound of formula (I-1), or (I-2), or a stereoisomer or tautomer thereof, or a pharmaceutically acceptable salt of any of the foregoing, Q¹ is selected from the group consisting of

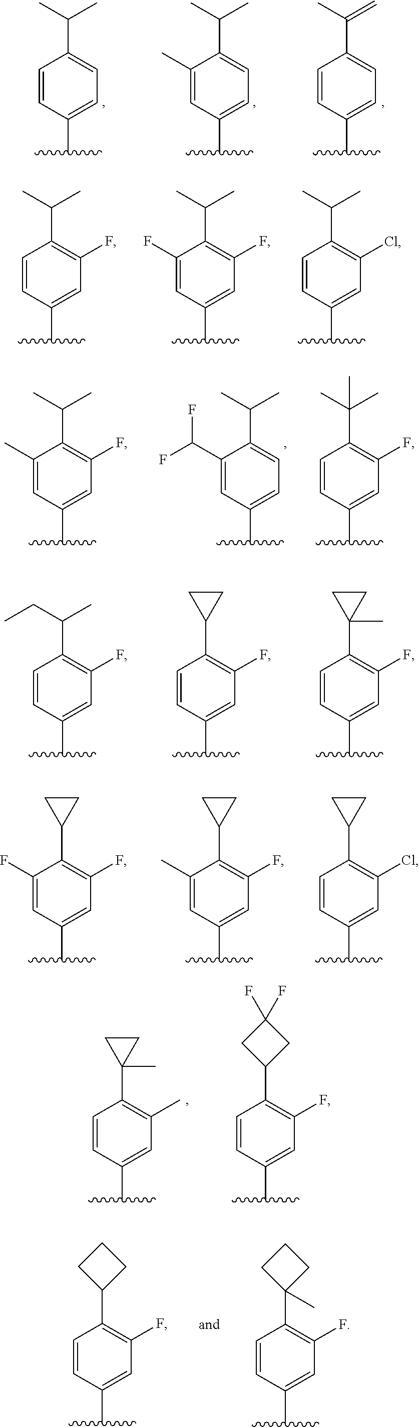

In some embodiments of a compound of formula (I-1), or (I-2), or a stereoisomer or tautomer thereof, or a pharmaceutically acceptable salt of any of the foregoing, Q¹ is selected from the group consisting of

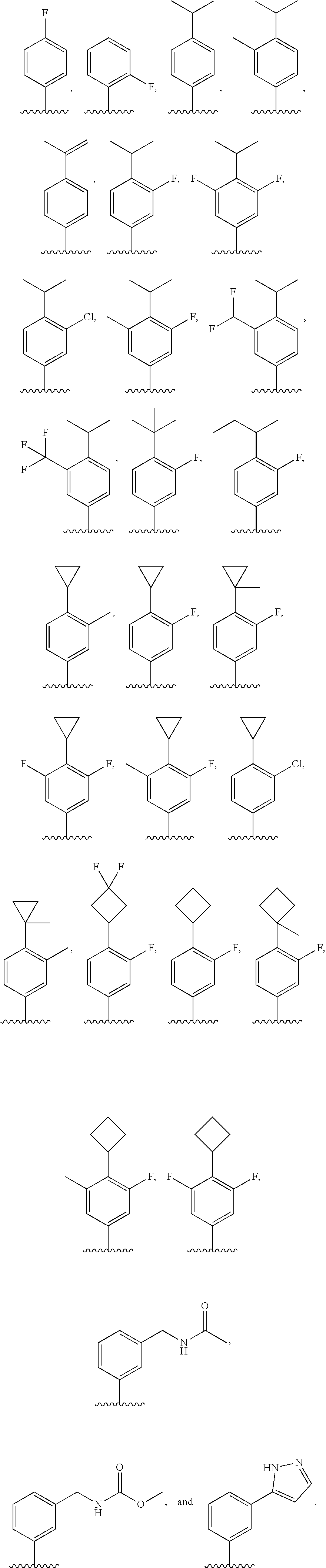

In some embodiments of a compound of formula (I-1), or (I-2), or a stereoisomer or tautomer thereof, or a pharmaceutically acceptable salt of any of the foregoing, Q¹ is selected from the group consisting of

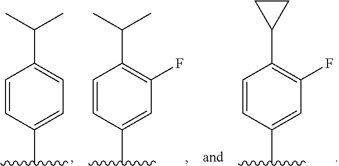

In some embodiments of a compound of formula (I-1), or (I-2), or a stereoisomer or tautomer thereof, or a pharmaceutically acceptable salt of any of the foregoing, Q¹ is 3-15 membered heterocyclyl, wherein the 3-15 membered heterocyclyl of Q¹ is optionally substituted with one or more oxo. In some embodiments, Q¹ is

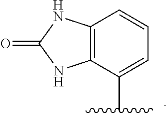 [SMILES: *c1cccc2[nH]c(=O)[nH]c12]

In some embodiments of a compound of formula (I-1), or (I-2), or a stereoisomer or tautomer thereof, or a pharmaceutically acceptable salt of any of the foregoing, Q¹ is (i) 3-15 membered heterocyclyl, wherein the 3-15 membered heterocyclyl of Q¹ is optionally substituted with one or more oxo, or C₁₋₆alkyl, (ii) 5-20 membered heteroaryl, wherein the 5-20 membered heteroaryl of Q¹ is optionally substituted with one or more halo, C₁₋₆alkyl, C₂₋₆alkenyl, C₁₋₆alkoxy, —NH₂, or C₃₋₁₀cycloalkyl, wherein, the C₁₋₆alkyl is optionally substituted with one or more halo, and the C₃₋₁₀cycloalkyl is optionally substituted with one or more halo or C₁₋₆alkyl, or (iii) C₃₋₁₀cycloalkyl.

In some embodiments of a compound of formula (I-1), or (I-2), or a stereoisomer or tautomer thereof, or a pharmaceutically acceptable salt of any of the foregoing, Q¹ is 3-15 membered heterocyclyl, wherein the 3-15 membered heterocyclyl of Q¹ is optionally substituted with one or more oxo, or C₁₋₆alkyl. In some embodiments Q¹ is selected from the group consisting of

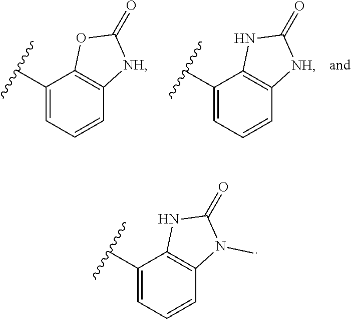 [SMILES: *c1cccc2[nH]c(=O)oc12 ; *c1cccc2[nH]c(=O)[nH]c12 ; *c1cccc2c1[nH]c(=O)n2C]

In some embodiments of a compound of formula (I-1), or (I-2), or a stereoisomer or tautomer thereof, or a pharmaceutically acceptable salt of any of the foregoing, Q¹ is 5-20 membered heteroaryl, wherein the 5-20 membered heteroaryl of Q¹ is optionally substituted with one or more halo, C₁₋₆alkyl, C₂₋₆alkenyl, C₁₋₆alkoxy, —NH₂, or C₃₋₁₀cycloalkyl, wherein, the C₁₋₆alkyl is optionally substituted with one or more halo, and the C₃₋₁₀cycloalkyl is optionally substituted with one or more halo or C₁₋₆alkyl. In some embodiments Q¹ is 5-20 membered heteroaryl, wherein the 5-20 membered heteroaryl of Q¹ is optionally substituted with one or more C₁₋₆alkyl. In some embodiments, is 5-20 membered heteroaryl, wherein the 5-20 membered heteroaryl of Q¹ comprises one or more annular N. In some embodiments, is 5-20 membered heteroaryl, wherein the 5-20 membered heteroaryl of Q¹ comprises two annular N. In some embodiments, is 5-20 membered heteroaryl, wherein the 5-20 membered heteroaryl of Q¹ is monocyclic of bicyclic. In some embodiments, is 5-20 membered heteroaryl, wherein the 5-20 membered heteroaryl of Q¹ is monocyclic. In some embodiments, is 5-20 membered heteroaryl, wherein the 5-20 membered heteroaryl of Q¹ is bicyclic. In some embodiments Q¹ is selected from the group consisting of

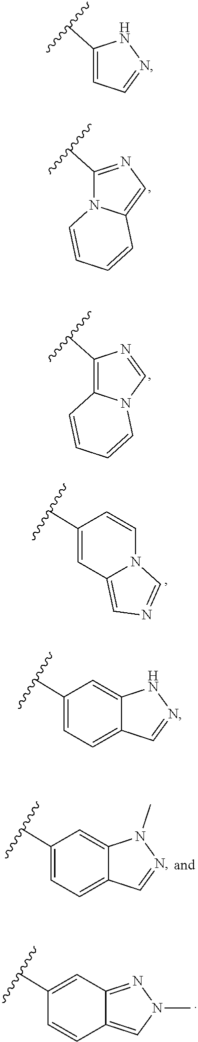

In some embodiments of a compound of formula (I-1), or (I-2), or a stereoisomer or tautomer thereof, or a pharmaceutically acceptable salt of any of the foregoing, Q¹ is C₃₋₁₀cycloalkyl. In some embodiments, Q¹ is C₃₋₆cycloalkyl. In some embodiments Q¹ is cyclopropyl.

In some embodiments of a compound of formula (I-1), or (I-2), or a stereoisomer or tautomer thereof, or a pharmaceutically acceptable salt of any of the foregoing, L¹ is absent or is —CH₂—. In some embodiments, L¹ is absent. In some embodiments, L¹ is —CH₂—. In some embodiments, L¹ is absent and Q¹ is C₃₋₁₀cycloalkyl. In some embodiments, L¹ is absent and Q¹ is C₃₋₆cycloalkyl. In some embodiments L¹ is absent and Q¹ is cyclopropyl. In some embodiments, L¹ is —CH₂— and Q¹ is C₃₋₁₀cycloalkyl. In some embodiments, L¹ is —CH₂— and Q¹ is C₃₋₆cycloalkyl. In some embodiments L¹ is —CH₂— and Q¹ is cyclopropyl.

In some embodiments of a compound of formula (I-1), or (I-2), or a stereoisomer or tautomer thereof, or a pharmaceutically acceptable salt of any of the foregoing, X¹, X², X³, X⁴, and X⁵ are each H.

In some embodiments of a compound of formula (I-1), or (I-2), or a stereoisomer or tautomer thereof, or a pharmaceutically acceptable salt of any of the foregoing, R¹ is H or C₁₋₆alkyl. In some embodiments R¹ is H. In some embodiments, R¹ is C₁₋₃alkyl. In some embodiments, R¹ is methyl.

In some embodiments of a compound of formula (I-1), or (I-2), or a stereoisomer or tautomer thereof, or a pharmaceutically acceptable salt of any of the foregoing, R^{k} is H, halo, —OH, —NH₂, or —NH—C(O)C₁₋₆alkyl. In some embodiments, R^{k} is H. In some embodiments, R^{k} is halo. In some embodiments, R^{k} is F.

In some embodiments of a compound of formula (I-1), or (I-2), or a stereoisomer or tautomer thereof, or a pharmaceutically acceptable salt of any of the foregoing, R^{m} is H, —OH, or C₁₋₆alkyl. In some embodiments R^{m} is H.

In some embodiments of a compound of formula (I-1), or (I-2), or a stereoisomer or tautomer thereof, or a pharmaceutically acceptable salt of any of the foregoing, Rⁿ is H, C₁₋₆alkyl, or C₃₋₁₀cycloalkyl. In some embodiments Rⁿ is H.

In some embodiments of a compound of formula (I-1), or (I-2), or a stereoisomer or tautomer thereof, or a pharmaceutically acceptable salt of any of the foregoing, R^{m} is H, Rⁿ is H, and R^{k} is H, halo, —OH, —NH₂, or —NH—C(O)C₁₋₆alkyl. In some embodiments, R^{m} is H, Rⁿ is H, and R^{k} is halo, —OH, or —NH₂. In some embodiments, R^{m} is H, Rⁿ is H, and R^{k} is halo. In some embodiments, R^{m} is H, Rⁿ is H, and R^{k} is fluoro.

In some embodiments of a compound of formula (I-1), or (I-2), or a stereoisomer or tautomer thereof, or a pharmaceutically acceptable salt of any of the foregoing, R^{k} is taken together with either R^{m} or Rⁿ, and the atoms to which they are attached, to form cyclopropyl.

In some embodiments of a compound of formula (I-1), or (I-2), or a stereoisomer or tautomer thereof, or a pharmaceutically acceptable salt of any of the foregoing, Rⁿ taken together with the carbon atom to which it is attached forms C₃₋₅ cycloalkyl. In some embodiments, Rⁿ taken together with the carbon atom to which it is attached forms cyclopropyl.

In some embodiments of a compound of formula (I-1), or (I-2), or a stereoisomer or tautomer thereof, or a pharmaceutically acceptable salt of any of the foregoing, R¹ is H.

In some embodiments of a compound of formula (I-1), or (I-2), or a stereoisomer or tautomer thereof, or a pharmaceutically acceptable salt of any of the foregoing, R² is C₁₋₆alkyl, wherein the C₁₋₆alkyl of R² is optionally substituted with one or more R^{a}. In some embodiments, R² is C₁₋₆alkyl, wherein the C₁₋₆alkyl of R² is substituted with one or more R^{a}, wherein R^{a} is —OH or 5-20 membered heteroaryl, wherein the 5-20 membered heteroaryl of R^{a} is optionally substituted with one or more R^{b}. In some embodiments, R² is methyl, wherein the methyl of R² is substituted with one or more R^{a}, wherein R^{a} is —OH or 5-10 membered heteroaryl, wherein the 5-10 membered heteroaryl of R^{a} is optionally substituted with one or more R^{b}. In some embodiments, R² is methyl, wherein the methyl of R² is substituted with one or more R^{a}, wherein R^{a} is —OH or 5-10 membered heteroaryl, wherein the 5-10 membered heteroaryl of R^{a} is optionally substituted with one or more C₁₋₆alkyl, wherein the C₁₋₆alkyl is optionally substituted with one or more halo, —NH₂, —NH(C₁₋₆alkyl), —N(C₁₋₆alkyl)₂, —NH—C(O)C₁₋₆alkyl, or —NH—C(O)—C₁₋₆alkoxy. In some embodiments, R² is methyl, wherein the methyl of R² is substituted with one or more R^{a}, wherein R^{a} is —OH or 5-10 membered heteroaryl, wherein the 5-10 membered heteroaryl of R^{a} is optionally substituted with one or more methyl, wherein the methyl is optionally substituted with one or more fluoro.

In some embodiments of a compound of formula (I-1), or (I-2), or a stereoisomer or tautomer thereof, or a pharmaceutically acceptable salt of any of the foregoing, R² is 5-20 membered heteroaryl or —(C₁₋₄alkyl)(5-20 membered heteroaryl), wherein the C₁₋₄ alkyl is optionally substituted with one or more or more —OH, halo, —NH₂, —NH(C₁₋₆alkyl), —N(C₁₋₆alkyl)₂, and wherein the 5-20 membered heteroaryl is optionally substituted with one or more R^{s}. In some embodiments, R² is 5-20 membered heteroaryl, wherein the C₁₋₄ alkyl is optionally substituted with one or more or more —OH, halo, —NH₂, —NH(C₁₋₆alkyl), —N(C₁₋₆alkyl)₂, and wherein the 5-20 membered heteroaryl is optionally substituted with one or more R^{s}. In some embodiments, R² is (methyl)(5-20 membered heteroaryl), wherein the methyl is optionally substituted with one or more or more —OH, halo, —NH₂, —NH(C₁₋₆alkyl), —N(C₁₋₆alkyl)₂, and wherein the 5-20 membered heteroaryl is optionally substituted with one or more R^{s}. In some embodiments, R^{s} is halo, C₁₋₆alkyl, C₁₋₆alkoxy, —NH₂, —NH(C₁₋₆alkyl), —N(C₁₋₆alkyl)₂, —NH—C(O)—C₁₋₆alkyl, C₆₋₂₀aryl, C₃₋₁₀cycloalkyl, 3-15 membered heterocyclyl, 5-20 membered heteroaryl, or —C(O)—C₁₋₆alkoxy. In some embodiments, the C₁₋₆alkyl of R^{s} is optionally substituted with one or more halo, C₁₋₆alkoxy, —NH₂, —NH(C₁₋₆alkyl), —N(C₁₋₆alkyl)₂, —NH—C(O)C₁₋₆alkyl, or —NH—C(O)—C₁₋₆alkoxy, and the 3-15-membered heterocyclyl of R^{s} is optionally substituted with one or more halo or —C(O)—C₁₋₆alkoxy.

In some embodiments of a compound of formula (I-1), or (I-2), or a stereoisomer or tautomer thereof, or a pharmaceutically acceptable salt of any of the foregoing, R² is selected from the group consisting of

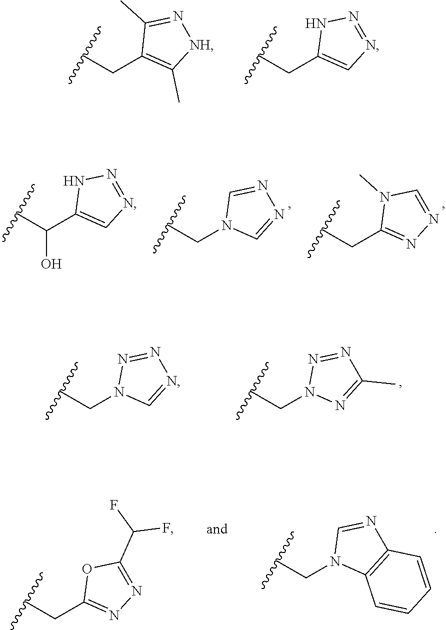

In some embodiments of a compound of formula (I), or a stereoisomer or tautomer thereof, or a pharmaceutically acceptable salt of any of the foregoing, R² is

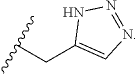 [SMILES: *Cc1cnn[nH]1]

In some embodiments of a compound of formula (I-1), or (I-2), or a stereoisomer or tautomer thereof, or a pharmaceutically acceptable salt of any of the foregoing, R² is selected from the group consisting of

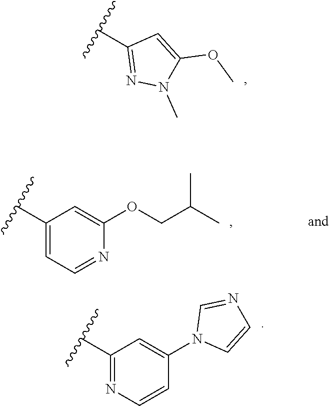

In some embodiments of a compound of formula (I-1), or (I-2), or a stereoisomer or tautomer thereof, or a pharmaceutically acceptable salt of any of the foregoing, R² is selected from the group consisting of

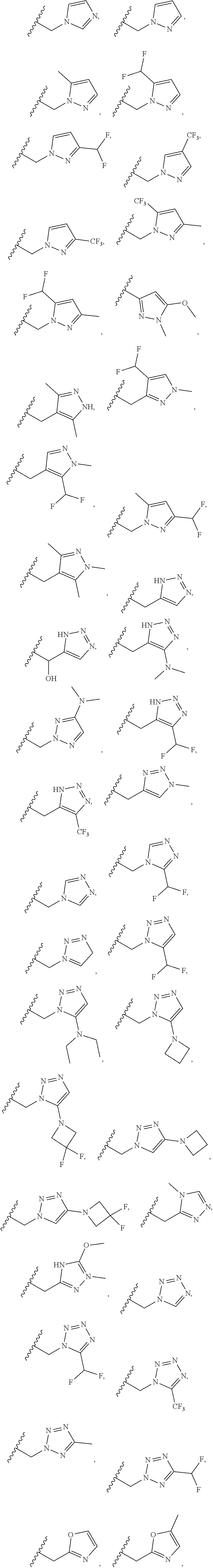

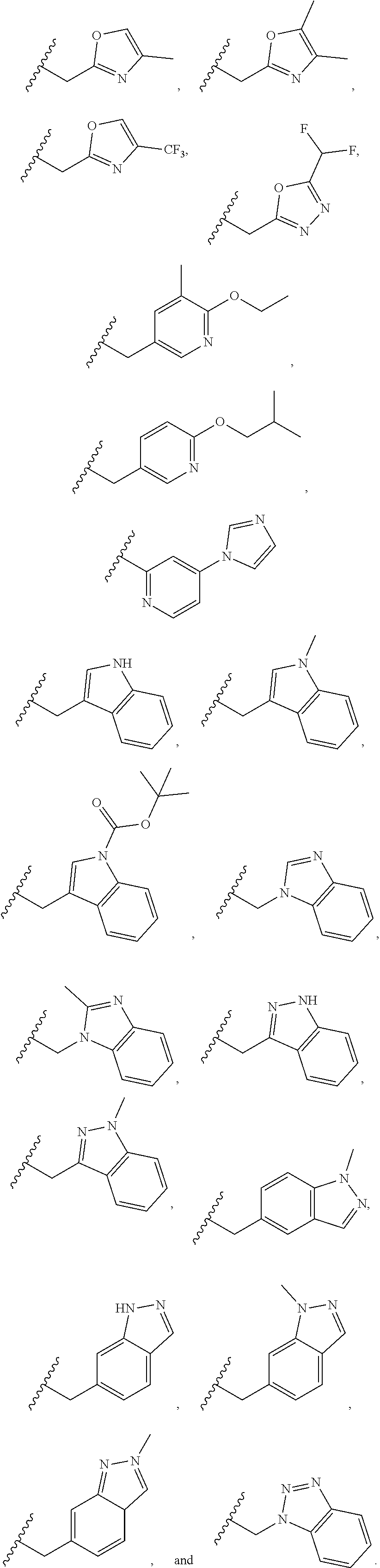

embodiments, R² is

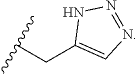 [SMILES: *Cc1cnn[nH]1]

In some embodiments of a compound of formula (I-1), or (I-2), or a stereoisomer or tautomer thereof, or a pharmaceutically acceptable salt of any of the foregoing, R² is C₁₋₆alkyl, wherein the C₁₋₆alkyl of R² is substituted with one or more R^{a}, wherein R^{a} is 3-15 membered heterocyclyl, wherein the 3-15 membered heterocyclyl of R^{a} is optionally substituted with one or more R^{c}.

In some embodiments of a compound of formula (I-1), or (I-2), or a stereoisomer or tautomer thereof, or a pharmaceutically acceptable salt of any of the foregoing, In some embodiments, R² is methyl, wherein the methyl of R² is substituted with one or more R^{a}, wherein R^{a} is 3-8 membered heterocyclyl, wherein the 3-8 membered heterocyclyl of R^{a} is optionally substituted with one or more R^{c}, wherein R^{c} is oxo, C₁₋₆alkyl, or —C(O)—C₁₋₆alkoxy, wherein the C₁₋₆alkyl of R^{c} is optionally substituted with one or more halo, and the —C(O)—C₁₋₆alkoxy of R^{c} is optionally substituted with one or more halo. In some embodiments, R² is selected from the group consisting of

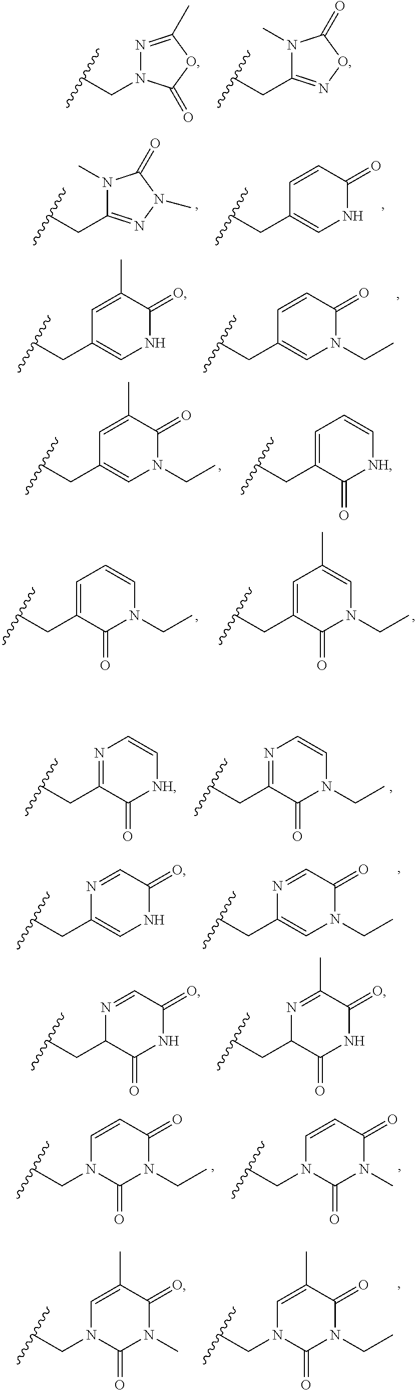

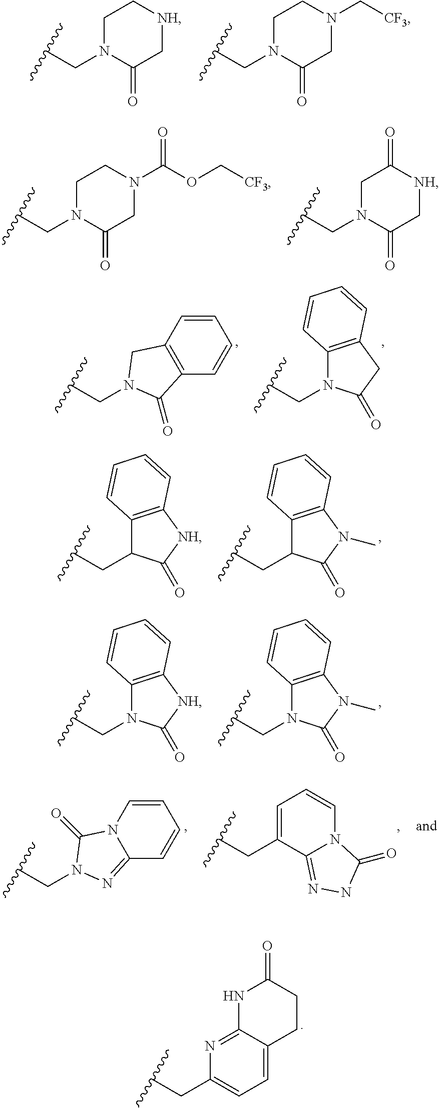

In some embodiments of a compound of formula (I-1), or (I-2), or a stereoisomer or tautomer thereof, or a pharmaceutically acceptable salt of any of the foregoing, R² is methyl, wherein the methyl of R² is substituted with one or more R^{a}, wherein R^{a} is —O—R^{e}, wherein R^{e} is —C(O)-(3-15 membered heterocyclyl) wherein the —C(O)-(3-15 membered heterocyclyl) of R^{e} is optionally substituted with one or more C₁₋₆alkyl, wherein the C₁₋₆alkyl is optionally substituted with one or more halo, C₁₋₆alkoxy, or C₃₋₁₀cycloalkyl. In some embodiments, R² is selected from the group consisting of

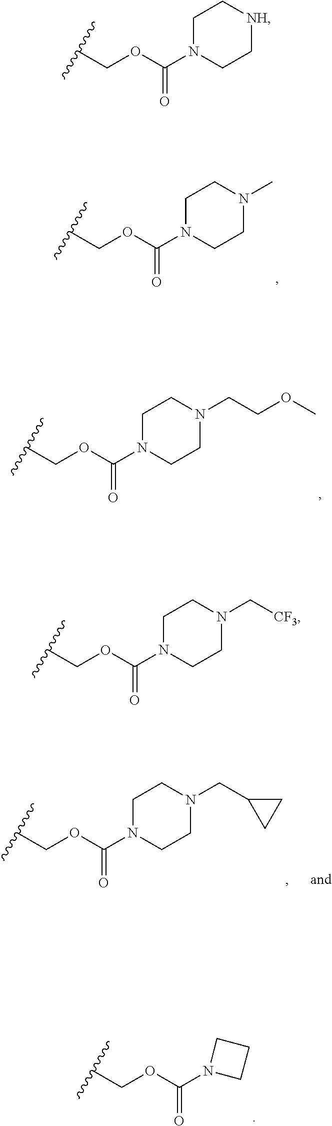 [SMILES: *COC(=O)N1CCNCC1 ; *COC(=O)N1CCN(C)CC1 ; *COC(=O)N1CCN(CCOC)CC1 ; *COC(=O)N1CCN(CC(F)(F)F)CC1 ; *COC(=O)N1CCN(CC2CC2)CC1 ; *COC(=O)N1CCC1]

In some embodiments of a compound of formula (I-1), or (I-2), or a stereoisomer or tautomer thereof, or a pharmaceutically acceptable salt of any of the foregoing, R² is methyl, wherein the methyl of R² is substituted with one or more R^{a}, wherein R^{a} is —N(R^{c})(R^{d}), wherein one of R^{c} and R^{d} is H, and the other of R^{c} and R^{d}, —C(O)—C₁₋₆alkyl, —C(O)—N(C₁₋₆alkyl)₂, or —C(O)-(3-15 membered heterocyclyl). In some embodiments, R² is selected from the group consisting of

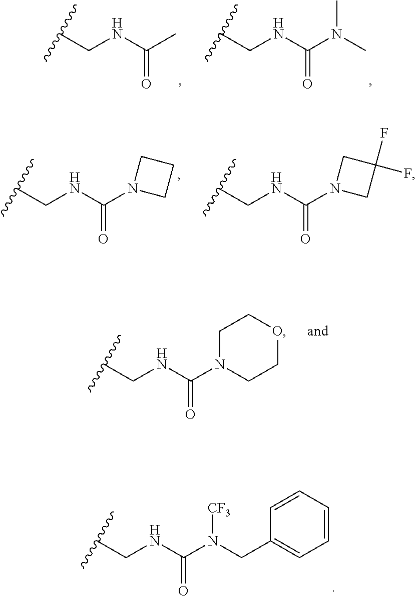

In some embodiments of a compound of formula (I-1), or (I-2), or a stereoisomer or tautomer thereof, or a pharmaceutically acceptable salt of any of the foregoing, R² is ethyl, wherein the ethyl of R² is substituted with one or more R^{a}, wherein R^{a} is —N(R^{c})(R^{d}), wherein one of R^{c} and R^{d} is H, and the other of R^{c} and R^{d}, is —C(O)—C₁₋₆alkyl. In some embodiments, R² is

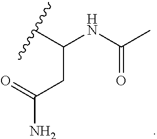 [SMILES: *C(CC(N)=O)NC(C)=O]

In some embodiments of a compound of formula (I-1), or (I-2), or a stereoisomer or tautomer thereof, or a pharmaceutically acceptable salt of any of the foregoing, X¹ and X² are each independently H, C₁₋₆alkyl, or C₁₋₆alkoxy. In some embodiments, X¹ and X² are each H.

In some embodiments of a compound of formula (I-1), or (I-2), or a stereoisomer or tautomer thereof, or a pharmaceutically acceptable salt of any of the foregoing, X³ and X⁴ are each independently H, halo, C₁₋₆alkyl, C₁₋₆alkoxy, or 5-20 membered heteroaryl, wherein the C₁₋₆alkyl of X³ and X⁴ is optionally substituted with one of more halo.

In some embodiments of a compound of formula (I-1), or (I-2), or a stereoisomer or tautomer thereof, or a pharmaceutically acceptable salt of any of the foregoing, X⁵ is H, C₁₋₆alkyl, C₁₋₆alkoxy, or C₃₋₁₀cycloalkyl. In some embodiments, X⁵ is H, C₁₋₄alkyl, C₁₋₃alkoxy, or C₃₋₆cycloalkyl. In some embodiments, X⁵ is H. In some embodiments, X⁵ is isopropyl, n-butyl, iso-butyl or t-butyl.

In some embodiments a compound of formula (I-1), or (I-2), or a stereoisomer or tautomer thereof, or a pharmaceutically acceptable salt of any of the foregoing, X¹—X⁵ are each H, and Q¹ is a 5-20 membered heteroaryl optionally substituted with one or more halo, C₁₋₆alkyl, —NH₂, or C₃₋₁₀cycloalkyl, wherein the C₃₋₁₀cycloalkyl is optionally substituted with one or more halo or C₁₋₆alkyl. In some embodiments, X¹—X⁵ are each H, and Q¹ is a 5-6 membered heteroaryl optionally substituted with one or more halo, C₁₋₆alkyl, —NH₂, or C₃₋₁₀cycloalkyl, wherein the C₃₋₁₀cycloalkyl is optionally substituted with one or more halo or C₁₋₆alkyl. In some embodiments, X¹—X⁵ are each H, and Q¹ is a pyridinyl optionally substituted with one or more halo, C₁₋₆alkyl, —NH₂, or C₃₋₁₀cycloalkyl, wherein the C₃₋₁₀cycloalkyl is optionally substituted with one or more halo or C₁₋₆alkyl. In some embodiments, X¹—X⁵ are each H, and Q¹ is a pyridinyl optionally substituted with one or more halo, C₁₋₄alkyl, —NH₂, or C₃₋₄cycloalkyl, wherein the C₃₋₄cycloalkyl is optionally substituted with one or more halo or C₁₋₆alkyl. In some embodiments of the foregoing, R^{m} is H and Rⁿ is H. In some embodiments of the foregoing, R¹ is H.

In some embodiments of a compound of formula (I-1), or (I-2), or a stereoisomer or tautomer thereof, or a pharmaceutically acceptable salt of any of the foregoing, or (I-2), X¹—X⁵ are each H, and Q¹ is phenyl substituted with one or more halo, C₁₋₆alkyl, C₂₋₆ alkenyl, —NH₂, —NH—C(O)—(C₁₋₆alkyl), —NH—C(O)-(3-15 membered heterocyclyl), or C₃₋₁₀cycloalkyl, wherein the C₁₋₆alkyl is optionally substituted with one or more halo, and the C₃₋₁₀cycloalkyl is optionally substituted with one or more halo or C₁₋₆alkyl. In some embodiments, X¹—X⁵ are each H, and Q¹ is phenyl substituted with one or more halo, C₁₋₆alkyl, C₂₋₆ alkenyl, —NH₂, —NH—C(O)—(C₁₋₆alkyl), —NH—C(O)-(3-10 membered heterocyclyl), or C₃₋₁₀cycloalkyl, wherein the C₁₋₆alkyl is optionally substituted with one or more halo, and the C₃₋₁₀cycloalkyl is optionally substituted with one or more halo or C₁₋₆alkyl. In some embodiments, X¹—X⁵ are each H, and Q¹ is phenyl substituted with one or more halo, C₁₋₄alkyl, C₂₋₄ alkenyl, —NH₂, —NH—C(O)—(C₁₋₄alkyl), —NH—C(O)-(3-10 membered heterocyclyl), or C₃₋₄cycloalkyl, wherein the C₁₋₄alkyl is optionally substituted with one or more halo, and the C₃₋₄cycloalkyl is optionally substituted with one or more halo or C₁₋₆alkyl.

In some embodiments of a compound of formula (I-1), or (I-2), or a stereoisomer or tautomer thereof, or a pharmaceutically acceptable salt of any of the foregoing, R¹ is H. In some embodiments of the foregoing, R^{m} is H and Rⁿ is H. In some embodiments of the foregoing, R^{k} is taken together with either R^{m} or Rⁿ, and the atoms to which they are attached, to form cyclopropyl.

In some embodiments of a compound of formula (I-1), or any embodiment or variation thereof, such as a compound of formula (I-A), (I-A1), (I-A2), (I-A3), (I-A4), (I-B), (I-B1), (I-B2), (I-C), (I-D), (I-D1), (I-D2), (I-E), (I-F), (I-G), or (I-H), or a stereoisomer or tautomer thereof, or a pharmaceutically acceptable salt of any of the foregoing, the moiety represented by

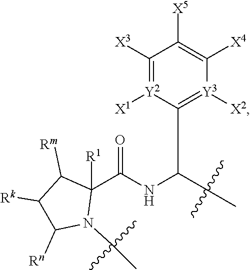

with carbon atoms bearing moieties

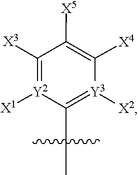

R^{k}, R^{m}, Rⁿ, and R¹, has a stereochemical configuration of the formula

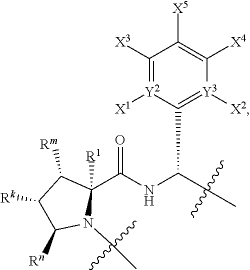

wherein X¹, X², X³, X⁴, X⁵, Y², Y³, R¹, R^{k}, R^{m}, and Rⁿ are as defined elsewhere herein.

In some embodiments of a compound of formula (I-1), or any embodiment or variation thereof, such as a compound of formula (I-A), (I-A1), (I-A2), (I-A3), (I-A4), (I-B), (I-B1), (I-B2), (I-C), (I-D), (I-D1), (I-D2), (I-E), (I-F), (I-G), or (I-H), or a stereoisomer or tautomer thereof, or a pharmaceutically acceptable salt of any of the foregoing, the moiety represented by

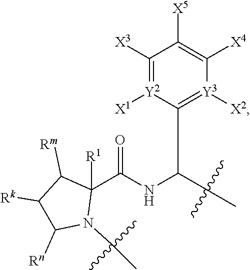

with carbon atoms bearing moieties

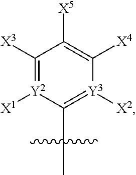

R^{k}, R^{m}, Rⁿ, and R¹, has a stereochemical configuration of the formula

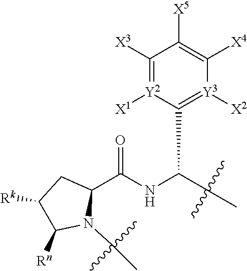

wherein R¹ and R^{m} are both H, and X¹, X², X³, X⁴, X⁵, Y², Y³, R^{k}, and Rⁿ are as defined elsewhere herein.

In some embodiments of a compound of formula (I-1), or any embodiment or variation thereof, such as a compound of formula (I-A), (I-A1), (I-A2), (I-A3), (I-A4), (I-B), (I-B1), (I-B2), (I-C), (I-D), (I-D1), (I-D2), (I-E), (I-F), (I-G), or (I-H), or a stereoisomer or tautomer thereof, or a pharmaceutically acceptable salt of any of the foregoing, the moiety represented by

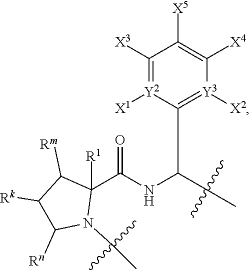

with carbon atoms bearing moieties

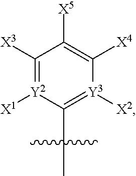

R^{k}, R^{m}, Rⁿ, and R¹, has a stereochemical configuration of the formula

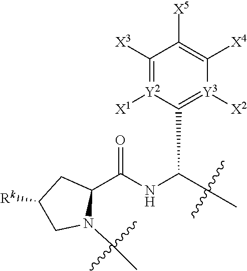

wherein R¹, R^{m}, and Rⁿ are each H, and X¹, X², X³, X⁴, X⁵, Y², Y³, and R^{k} are as defined elsewhere herein.

In some embodiments of a compound of formula (I-1), or any embodiment or variation thereof, such as a compound of formula (I-A), (I-A1), (I-A2), (I-A3), (I-A4), (I-B), (I-B1), (I-B2), (I-C), (I-D), (I-D1), (I-D2), (I-E), (I-F), (I-G), or (I-H), or a stereoisomer or tautomer thereof, or a pharmaceutically acceptable salt of any of the foregoing, the moiety represented by

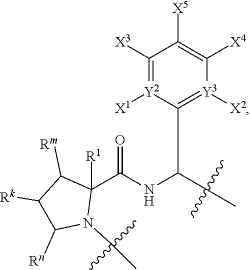

with carbon atoms bearing moieties

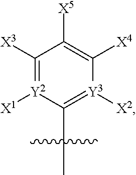

R^{k}, R^{m}, Rⁿ, and R¹, has a stereochemical configuration of the formula

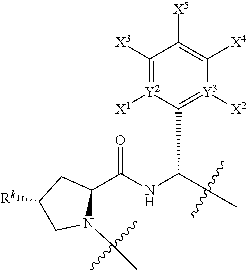

wherein R¹, R^{m}, and Rⁿ are each H, R^{k} is halo or H, and X¹, X², X³, X⁴, X⁵, Y², and Y³ are as defined elsewhere herein. In some embodiments, the moiety R^{k} is fluoro.

In some embodiments of a compound of formula (I-1), or any embodiment or variation thereof, such as a compound of formula (I-A), (I-A1), (I-A2), (I-A3), (I-A4), (I-B), (I-B1), (I-B2), (I-C), (I-D), (I-D1), (I-D2), (I-E), (I-F), (I-G), or (I-H), or a stereoisomer or tautomer thereof, or a pharmaceutically acceptable salt of any of the foregoing, the moiety represented by

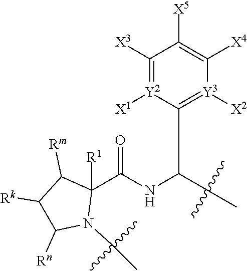

with carbon atoms beaning moieties

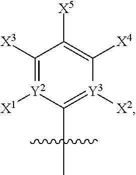

R^{k}, R^{m}, Rⁿ, and R¹, has a stereochemical configuration of the formula

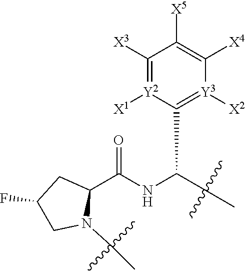

wherein R¹, R^{m}, and Rⁿ are each H, R^{k} is fluoro, and X¹, X², X³, X⁴, X⁵, Y², and Y³ are as defined elsewhere herein. In some embodiments Y² and Y³ are each C. In some embodiments one Y² and Y³ is C and the other of Y² and Y³ is N.

In some embodiments, provided herein is a compound of formula (I-1), or a stereoisomer or tautomer thereof, or a pharmaceutically acceptable salt thereof, wherein the compound is a compound of formula (I-A):

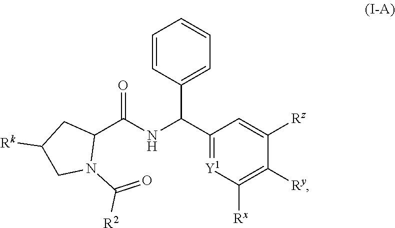

or a stereoisomer or tautomer thereof, or a pharmaceutically acceptable salt of any of the foregoing, wherein Y¹ is CH or N; R^{x} and R^{z} are independently H, halo, C₁₋₆alkyl, or —NH₂, wherein, when Y¹ is CH, the C₁₋₆alkyl of R^{x} or R^{z} may be optionally substituted with one or more halo; and R^{y} is (i) C₁₋₆alkyl, (ii), C₂₋₆alkenyl, or (iii) C₃₋₁₀cycloalkyl, wherein the C₃₋₁₀cycloalkyl is optionally substituted with one or more halo or C₁₋₆alkyl.

In some embodiments of a compound of formula (I-A), or a stereoisomer or tautomer thereof, or a pharmaceutically acceptable salt of any of the foregoing, R^{x} and R^{z} are independently H, fluoro, chloro, or methyl; and R^{y} is (i) isopropyl, (ii) isopropenyl, or (iii) C₃₋₄cycloalkyl, wherein the C₃₋₄cycloalkyl is optionally substituted with one or more fluoro or methyl. In some embodiments, R^{x} and R^{z} are independently H, fluoro, chloro, or methyl; and R^{y} is (i) isopropyl or (ii) C₃₋₄cycloalkyl, wherein the C₃₋₄cycloalkyl is optionally substituted with one or more fluoro or methyl. In some embodiments, R^{x} is H, fluoro, chloro, or methyl; R^{z} is H; and R^{y} is (i) isopropyl, or (ii) C₃₋₄cycloalkyl, wherein the C₃₋₄cycloalkyl is optionally substituted with one or more fluoro or methyl.

In some embodiments of a compound of formula (I-A), or a stereoisomer or tautomer thereof, or a pharmaceutically acceptable salt of any of the foregoing, R^{x} is fluoro or methyl optionally substituted with one or more fluoro; R^{y} is (i) isopropyl (ii) isopropenyl or (iii) C₃₋₄cycloalkyl optionally substituted with one or more halo or C₁₋₆alkyl or (iv) butyl; and R^{z} is fluoro or methyl; provided that at least one of R^{x} and R^{z} is halo, CF₂ or CF₃.

In some embodiments of a compound of formula (I-A), or a stereoisomer or tautomer thereof, or a pharmaceutically acceptable salt of any of the foregoing, R^{k} is H or halo. In some embodiments of a compound of formula (I-A), or a stereoisomer or tautomer thereof, or a pharmaceutically acceptable salt of any of the foregoing, R^{k} is H or fluoro. In some embodiments of a compound of formula (I-A), or a stereoisomer or tautomer thereof, or a pharmaceutically acceptable salt of any of the foregoing, R^{k} is fluoro.

In some embodiments of a compound of formula (I-A), or a stereoisomer or tautomer thereof, or a pharmaceutically acceptable salt of any of the foregoing, R² is C₁₋₆alkyl, wherein the C₁₋₆alkyl of R² is optionally substituted with one or more R^{a}. In some embodiments, R² is C₁₋₆alkyl, wherein the C₁₋₆alkyl of R² is substituted with one or more R^{a}, wherein R^{a} is —OH or 5-20 membered heteroaryl, wherein the 5-20 membered heteroaryl of R^{a} is optionally substituted with one or more R^{b}. In some embodiments, R² is methyl, wherein the methyl of R² is substituted with one or more R^{a}, wherein R^{a} is —OH or 5-10 membered heteroaryl, wherein the 5-10 membered heteroaryl of R^{a} is optionally substituted with one or more R^{b}. In some embodiments, R² is methyl, wherein the methyl of R² is substituted with one or more R^{a}, wherein R^{a} is —OH or 5-10 membered heteroaryl, wherein the 5-10 membered heteroaryl of R^{a} is optionally substituted with one or more C₁₋₆alkyl, wherein the C₁₋₆alkyl is optionally substituted with one or more halo, —NH₂, —NH(C₁₋₆alkyl), —N(C₁₋₆alkyl)₂, —NH—C(O)C₁₋₆alkyl, or —NH—C(O)—C₁₋₆alkoxy. In some embodiments, R² is methyl, wherein the methyl of R² is substituted with one or more R^{a}, wherein R^{a} is —OH or 5-10 membered heteroaryl, wherein the 5-10 membered heteroaryl of R^{a} is optionally substituted with one or more methyl, wherein the methyl is optionally substituted with one or more fluoro.

In some embodiments of a compound of formula (I-A), or a stereoisomer or tautomer thereof, or a pharmaceutically acceptable salt of any of the foregoing, R² is 5-20 membered heteroaryl or —(C₁₋₄alkyl)(5-20 membered heteroaryl), wherein the C₁₋₄ alkyl is optionally substituted with one or more or more —OH, halo, —NH₂, —NH(C₁₋₆alkyl), —N(C₁₋₆alkyl)₂, and wherein the 5-20 membered heteroaryl is optionally substituted with one or more R^{s}. In some embodiments, R^{s} is halo, C₁₋₆alkyl, C₁₋₆alkoxy, —NH₂, —NH(C₁₋₆alkyl), —N(C₁₋₆alkyl)₂, —NH—C(O)—C₁₋₆alkyl, C₆₋₂₀aryl, C₃₋₁₀cycloalkyl, 3-15 membered heterocyclyl, 5-20 membered heteroaryl, or —C(O)—C₁₋₆alkoxy. In some embodiments, the C₁₋₆alkyl of R^{s} is optionally substituted with one or more halo, C₁₋₆alkoxy, —NH₂, —NH(C₁₋₆alkyl), —N(C₁₋₆alkyl)₂, —NH—C(O)C₁₋₆alkyl, or —NH—C(O)—C₁₋₆alkoxy, and the 3-15-membered heterocyclyl of R^{s} is optionally substituted with one or more halo or —C(O)—C₁₋₆alkoxy.

In some embodiments of a compound of formula (I-A), or a stereoisomer or tautomer thereof, or a pharmaceutically acceptable salt of any of the foregoing, R² is selected from the group consisting of

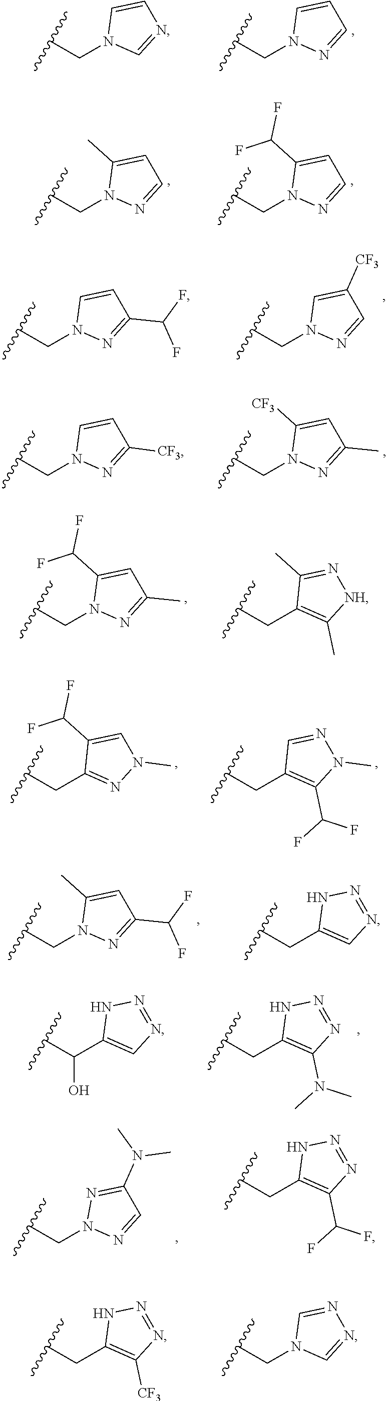

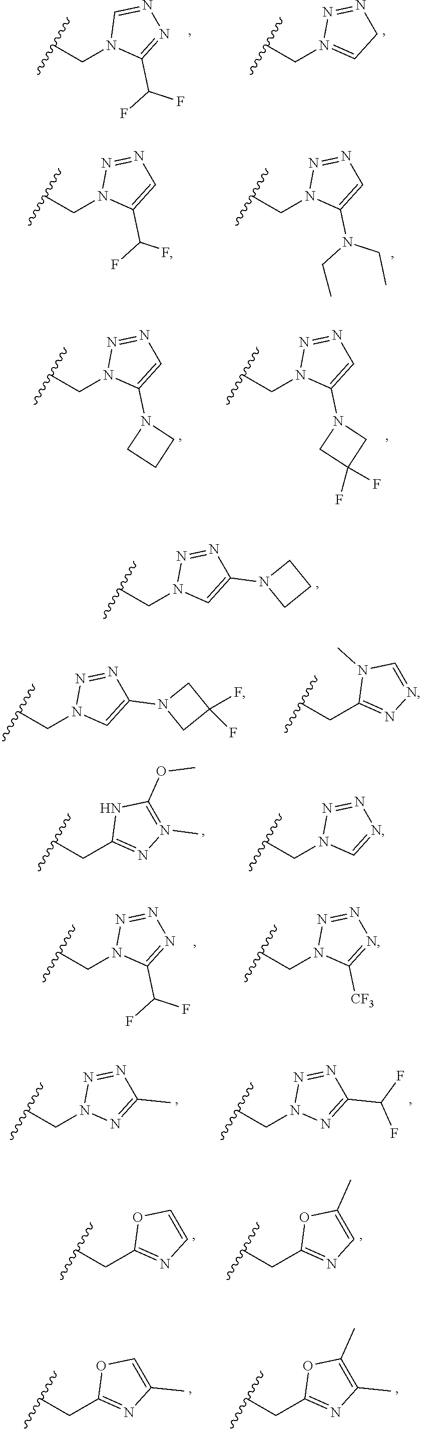

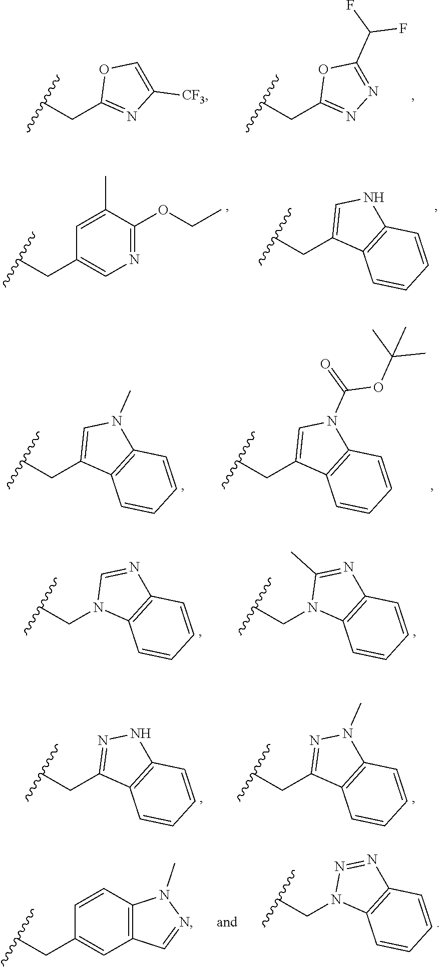

In some embodiments, R² is

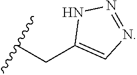 [SMILES: *Cc1cnn[nH]1]

In some embodiments of a compound of formula (I-A), or a stereoisomer or tautomer thereof, or a pharmaceutically acceptable salt of any of the foregoing, In some embodiments, R² is methyl, wherein the methyl of R² is substituted with one or more R^{a}, wherein R^{a} is 3-8 membered heterocyclyl, wherein the 3-8 membered heterocyclyl of R^{a} is optionally substituted with one or more R^{c}, wherein R^{c} is oxo, C₁₋₆alkyl, or —C(O)—C₁₋₆alkoxy, wherein the C₁₋₆alkyl of R^{c} is optionally substituted with one or more halo, and the —C(O)—C₁₋₆alkoxy of R^{c} is optionally substituted with one or more halo. In some embodiments, R² is selected from the group consisting of

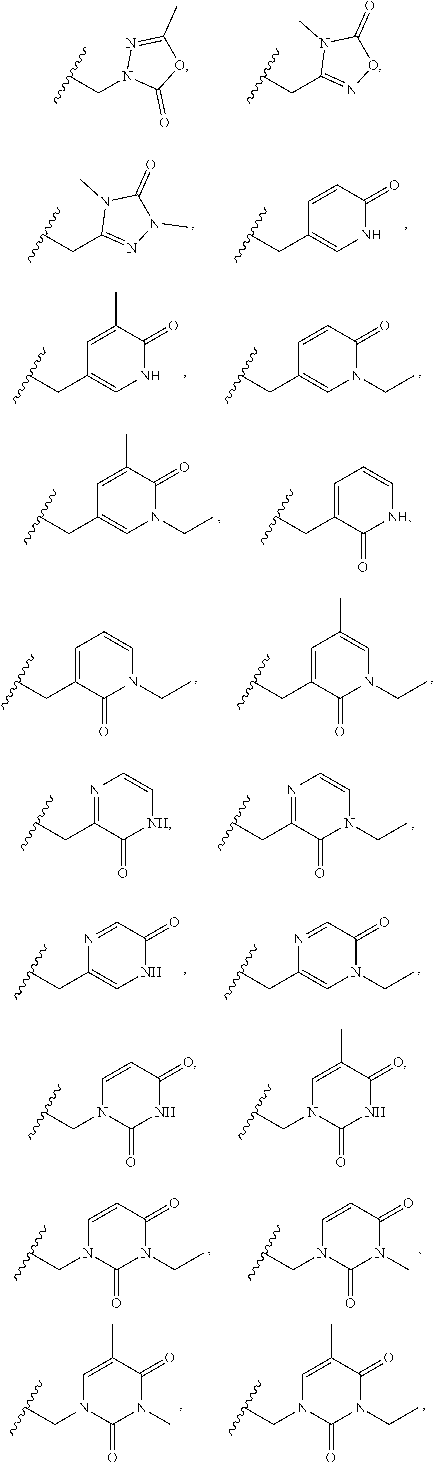

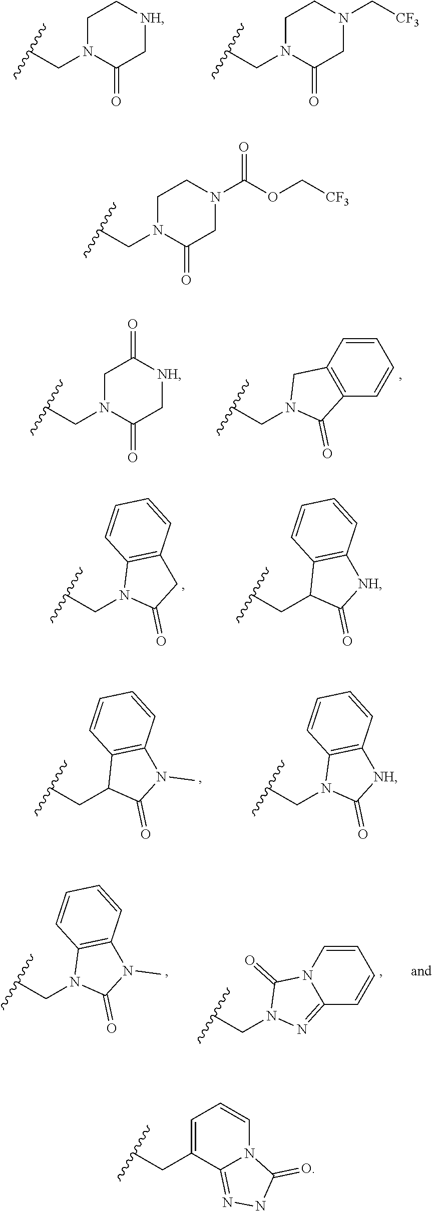

In some embodiments of a compound of formula (I-A), or a stereoisomer or tautomer thereof, or a pharmaceutically acceptable salt of any of the foregoing, R² is methyl, wherein the methyl of R² is substituted with one or more R^{a}, wherein R^{a} is —O—R^{e}, wherein R^{e} is —C(O)-(3-15 membered heterocyclyl) wherein the —C(O)-(3-15 membered heterocyclyl) of R^{e} is optionally substituted with one or more C₁₋₆alkyl, wherein the C₁₋₆alkyl is optionally substituted with one or more halo, C₁₋₆alkoxy, or C₃₋₁₀cycloalkyl. In some embodiments, R² is selected from the group consisting of

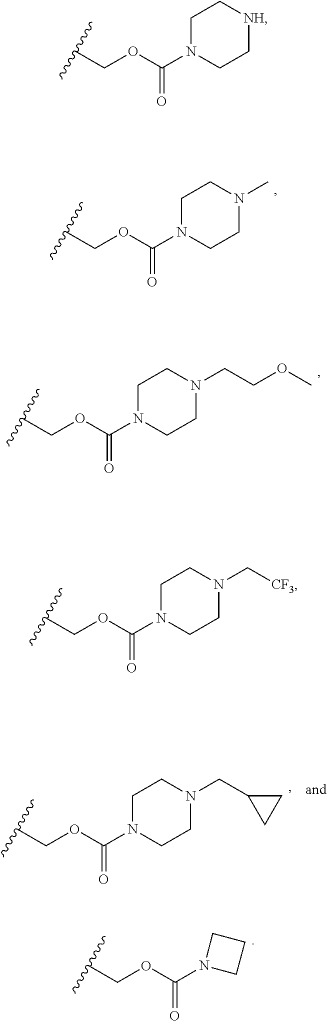 [SMILES: *COC(=O)N1CCNCC1 ; *COC(=O)N1CCN(C)CC1 ; *COC(=O)N1CCN(CCOC)CC1 ; *COC(=O)N1CCN(CC(F)(F)F)CC1 ; *COC(=O)N1CCN(CC2CC2)CC1 ; *COC(=O)N1CCC1]

In some embodiments of a compound of formula (I-A), or a stereoisomer or tautomer thereof, or a pharmaceutically acceptable salt of any of the foregoing, R² is methyl, wherein the methyl of R² is substituted with one or more R^{a}, wherein R^{a} is —N(R^{c})(R^{d}), wherein one of R^{c} and R^{d} is H, and the other of R^{c} and R^{d}, —C(O)—C₁₋₆alkyl, —C(O)—N(C₁₋₆alkyl)₂, or —C(O)-(3-15 membered heterocyclyl). In some embodiments, R² is selected from the group consisting of

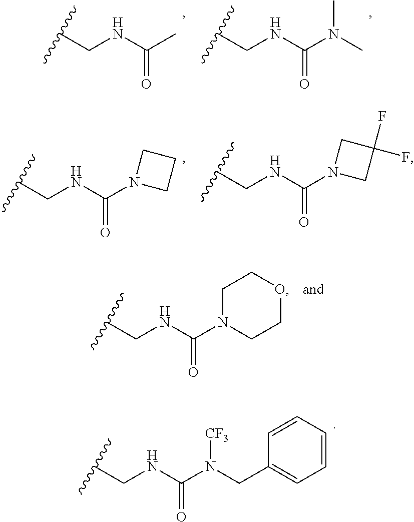

In some embodiments of a compound of formula (I-A), or a stereoisomer or tautomer thereof, or a pharmaceutically acceptable salt of any of the foregoing, R² is ethyl, wherein the ethyl of R² is substituted with one or more R^{a}, wherein R^{a} is —N(R)(R^{d}), wherein one of R^{c} and R^{d} is H, and the other of R^{c} and R^{d}, is —C(O)—C₁₋₆alkyl. In some embodiments, R² is

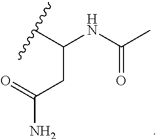 [SMILES: *C(CC(N)=O)NC(C)=O]

In some embodiments of a compound of formula (I-A), or a stereoisomer or tautomer thereof, or a pharmaceutically acceptable salt of any of the foregoing, Y¹ is CH or N; R^{x} and R^{z} are independently H or halo; R^{y} is C₁₋₆alkyl or C₃₋₁₀cycloalkyl; R^{k} is H or halo; and R² is selected from (i) to (iii):
(i) C₁₋₆alkyl, wherein the C₁₋₆alkyl of R² is substituted with one or more R^{a}, wherein R^{a} is
   (a) —OH,
   (b) C₆₋₂₀aryl, wherein the C₆₋₂₀aryl of R^{a} is optionally substituted with one or more halo, cyano, C₁₋₆alkoxy, or —NH—C(O)—C₁₋₆alkyl,
   (c) 3-15 membered heterocyclyl, wherein the 3-15 membered heterocyclyl of R^{a} is optionally substituted with one or more R^{c}, wherein
      R^{c} is halo, oxo, C₁₋₆alkyl, C₁₋₆alkoxy, —C(O)—C₁₋₆alkyl, or —C(O)—C₁₋₆alkoxy, wherein
         the C₁₋₆alkyl of R^{c} is optionally substituted with one or more halo or C₂₋₆alkynyl, and
         the —C(O)—C₁₋₆alkoxy of R^{c} is optionally substituted with one or more halo,
   (d) —N(R^{c})(R^{d}), wherein R^{c} and R^{d} of N(R^{c})(R^{d}) are, independently of each other, H, C₁₋₆alkyl, —C(O)—C₁₋₆alkyl, —C(O)—C₁₋₆alkoxy, —C(O)—NH₂, —C(O)—NH(C₁₋₆alkyl), —C(O)—N(C₁₋₆alkyl)₂, —C(O)-(3-15 membered heterocyclyl), —CH₂—C(O)—NH₂, 3-15 membered heterocyclyl, or 5-20 membered heteroaryl, wherein the C₁₋₆alkyl of R^{c} or R^{d} is optionally substituted with one or more —C(O)—NH₂,
      the —C(O)—C₁₋₆alkyl of R^{c} or R^{d} is optionally substituted with one or more halo,
      the 3-15 membered heterocyclyl and the 5-20 membered heteroaryl of R^{c} or R^{d} are independently optionally substituted with one or more C₁₋₆alkyl,
      the —C(O)-(3-15 membered heterocyclyl) of R^{c} or R^{d} is optionally substituted with one or more halo, —C(O)—C₁₋₆alkoxy, or C₁₋₆alkyl, wherein the C₁₋₆alkyl is optionally substituted with one or more halo, C₁₋₆alkoxy, or C₃₋₁₀cycloalkyl, and
      the C₁₋₆alkyl of the —C(O)—N(C₁₋₆alkyl)₂ of R^{c} or R^{d} are, independently of each other, optionally substituted with one or more halo or C₆₋₂₀aryl,
   (e) —O—R^{e}, wherein R^{e} is C₁₋₆alkyl, C₆₋₂₀aryl, —C(O)-(3-15 membered heterocyclyl), —C(O)—N—(C₁₋₆alkyl)₂, or 5-20 membered heteroaryl, wherein
      the C₁₋₆alkyl of R^{e} is optionally substituted with one or more C₁₋₆alkoxy, wherein the C₁₋₆alkoxy is optionally substituted with one or more C₂₋₆alkynyl,
      the C₆₋₂₀aryl of R^{e} is optionally substituted with one or more C₁₋₆alkyl, and
      the —C(O)-(3-15 membered heterocyclyl) of R^{e} is optionally substituted with one or more C₁₋₆alkyl, C₁₋₆alkoxy, or —C(O)—C₁₋₆alkoxy, wherein the C₁₋₆alkyl is optionally substituted with one or more halo, C₁₋₆alkoxy, or C₃₋₁₀cycloalkyl, or
   (f) —C(O)—R^{e}, wherein R^{e} of —C(O)—R^{e} is —NH₂, —OH, or 3-15 membered heterocyclyl,
(ii) 3-15 membered heterocyclyl, wherein the 3-15 membered heterocyclyl of R² is optionally substituted with one or more halo, oxo, C₁₋₆alkyl, —C(O)—C₁₋₆alkyl, or 5-20 membered heteroaryl,
(iii) 5-20 membered heteroaryl or —(C₁₋₄alkyl)(5-20 membered heteroaryl), wherein the C₁₋₄ alkyl is optionally substituted with one or more or more —OH, halo, —NH₂, —NH(C₁₋₆alkyl), —N(C₁₋₆alkyl)₂, and wherein the 5-20 membered heteroaryl is optionally substituted with one or more R^{s}, wherein
   R^{s} is halo, C₁₋₆alkyl, C₁₋₆alkoxy, —NH₂, —NH(C₁₋₆alkyl), —N(C₁₋₆alkyl)₂, —NH—C(O)—C₁₋₆alkyl, C₆₋₂₀aryl, C₃₋₁₀cycloalkyl, 3-15 membered heterocyclyl, 5-20 membered heteroaryl, or —C(O)—C₁₋₆alkoxy, wherein
      the C₁₋₆alkyl of R^{s} is optionally substituted with one or more halo, C₁₋₆alkoxy, —NH₂, —NH(C₁₋₆alkyl), —N(C₁₋₆alkyl)₂, —NH—C(O)C₁₋₆alkyl, or —NH—C(O)—C₁₋₆alkoxy, and
      the 3-15-membered heterocyclyl of R^{s} is optionally substituted with one or more halo or —C(O)—C₁₋₆alkoxy.

In some embodiments of formula (I-A), Y¹ is CH or N; R^{x} and R^{z} are independently H or halo; R^{y} is C₁₋₆alkyl or C₃₋₁₀cycloalkyl; R^{k} is H or halo; R² is C₁₋₆alkyl, wherein the C₁₋₆alkyl of R² is substituted with one or more R^{a}; R^{a} is
(a) —OH,
(b) C₆₋₁₀aryl optionally substituted with one or more halo, cyano, C₁₋₃alkoxy, or —NH—C(O)—C₁₋₃alkyl, or
(c) 3-15 membered heterocyclyl optionally substituted with one or more halo, oxo, C₁₋₆alkyl, C₁₋₆alkoxy, —C(O)—C₁₋₆alkyl, or —C(O)—C₁₋₆alkoxy.

In some embodiments of formula (I-A), Y¹ is CH or N; R^{x} and R^{z} are independently H or halo; R^{y} is C₁₋₃alkyl or C₃₋₅cycloalkyl; R^{k} is halo; R² is C₁₋₄alkyl substituted with one or more R^{a}; R^{a} is
(a) —OH,
(b) C₆₋₁₀aryl optionally substituted with one or more halo, cyano, C₁₋₃alkoxy, or —NH—C(O)—C₁₋₃alkyl, or
(c) C₃₋₈heteroaryl optionally substituted with one or more halo, oxo, C₁₋₆alkyl, C₁₋₆alkoxy, —C(O)—C₁₋₆alkyl, or —C(O)—C₁₋₆alkoxy.

In some embodiments, provided herein is a compound of formula (I-1) or formula (I-A), or a stereoisomer or tautomer thereof, or a pharmaceutically acceptable salt thereof, wherein the compound is of formula (I-A1):

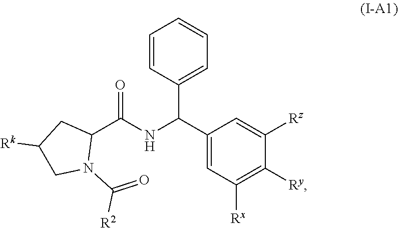

or a stereoisomer or tautomer thereof, or a pharmaceutically acceptable salt of any of the foregoing, wherein R^{x} and R^{z} are independently H, halo, C₁₋₆alkyl, or —NH₂, wherein the C₁₋₆alkyl is optionally substituted with one or more halo. In some embodiments, R^{x} is H, halo, or C₁₋₆alkyl; R^{y} is (i) C₁₋₆alkyl, (ii) C₂₋₆alkenyl, or (ii) C₃₋₁₀cycloalkyl; and R^{z} is H, halo or C₁₋₆alkyl. In some embodiments, R^{z} is H. In some embodiments, at least one of R^{x} and R^{z} is halo.

In some embodiments of a compound of formula (I-A1), or a stereoisomer or tautomer thereof, or a pharmaceutically acceptable salt of any of the foregoing, R^{x} is fluoro or methyl; R^{y}is (i) isopropyl or (ii) C₃₋₄cycloalkyl; and R^{z} is fluoro or methyl; provided that at least one of R^{x} and R^{z} is halo.

In some embodiments of a compound of formula (I-A1), or a stereoisomer or tautomer thereof, or a pharmaceutically acceptable salt of any of the foregoing, R^{x} is fluoro or methyl optionally substituted with one or more fluoro; R^{y} is (i) isopropyl (ii) C₃₋₄cycloalkyl optionally substituted with one or more halo or C₁₋₆alkyl or (iii) butyl; and R^{z} is fluoro or methyl; provided that at least one of R^{x} and R^{z} is halo, CF₂ or CF₃.

In some embodiments of a compound of formula (I-A1), or a stereoisomer or tautomer thereof, or a pharmaceutically acceptable salt of any of the foregoing, R^{k} is H or halo. In some embodiments of a compound of formula (I-A1), or a stereoisomer or tautomer thereof, or a pharmaceutically acceptable salt of any of the foregoing, R^{k} is H or fluoro. In some embodiments of a compound of formula (I-A1), or a stereoisomer or tautomer thereof, or a pharmaceutically acceptable salt of any of the foregoing, R^{k} is fluoro.

In some embodiments of a compound of formula (I-A1), or a stereoisomer or tautomer thereof, or a pharmaceutically acceptable salt of any of the foregoing, R² is C₁₋₆alkyl, wherein the C₁₋₆alkyl of R² is optionally substituted with one or more R^{a}. In some embodiments, R² is C₁₋₆alkyl, wherein the C₁₋₆alkyl of R² is substituted with one or more R^{a}, wherein R^{a} is —OH or 5-20 membered heteroaryl, wherein the 5-20 membered heteroaryl of R^{a} is optionally substituted with one or more R^{b}. In some embodiments, R² is methyl, wherein the methyl of R² is substituted with one or more R^{a}, wherein R^{a} is —OH or 5-10 membered heteroaryl, wherein the 5-10 membered heteroaryl of R^{a} is optionally substituted with one or more R^{b}. In some embodiments, R² is methyl, wherein the methyl of R² is substituted with one or more R^{a}, wherein R^{a} is —OH or 5-10 membered heteroaryl, wherein the 5-10 membered heteroaryl of R^{a} is optionally substituted with one or more C₁₋₆alkyl, wherein the C₁₋₆alkyl is optionally substituted with one or more halo, —NH₂, —NH(C₁₋₆alkyl), —N(C₁₋₆alkyl)₂, —NH—C(O)C₁₋₆alkyl, or —NH—C(O)—C₁₋₆alkoxy. In some embodiments, R² is methyl, wherein the methyl of R² is substituted with one or more R^{a}, wherein R^{a} is —OH or 5-10 membered heteroaryl, wherein the 5-10 membered heteroaryl of R^{a} is optionally substituted with one or more methyl, wherein the methyl is optionally substituted with one or more fluoro.

In some embodiments of a compound of formula (I-A1), or a stereoisomer or tautomer thereof, or a pharmaceutically acceptable salt of any of the foregoing, R² is 5-20 membered heteroaryl or —(C₁₋₄alkyl)(5-20 membered heteroaryl), wherein the C₁₋₄ alkyl is optionally substituted with one or more or more —OH, halo, —NH₂, —NH(C₁₋₆alkyl), —N(C₁₋₆alkyl)₂, and wherein the 5-20 membered heteroaryl is optionally substituted with one or more R^{s}. In some embodiments, R^{s} is halo, C₁₋₆alkyl, C₁₋₆alkoxy, —NH₂, —NH(C₁₋₆alkyl), —N(C₁₋₆alkyl)₂, —NH—C(O)—C₁₋₆alkyl, C₆₋₂₀aryl, C₃₋₁₀cycloalkyl, 3-15 membered heterocyclyl, 5-20 membered heteroaryl, or —C(O)—C₁₋₆alkoxy. In some embodiments, the C₁₋₆alkyl of R^{s} is optionally substituted with one or more halo, C₁₋₆alkoxy, —NH₂, —NH(C₁₋₆alkyl), —N(C₁₋₆alkyl)₂, —NH—C(O)C₁₋₆alkyl, or —NH—C(O)—C₁₋₆alkoxy, and the 3-15-membered heterocyclyl of R^{s} is optionally substituted with one or more halo or —C(O)—C₁₋₆alkoxy.

In some embodiments of a compound of formula (I-A1), or a stereoisomer or tautomer thereof, or a pharmaceutically acceptable salt of any of the foregoing, R² is selected from the group consisting of

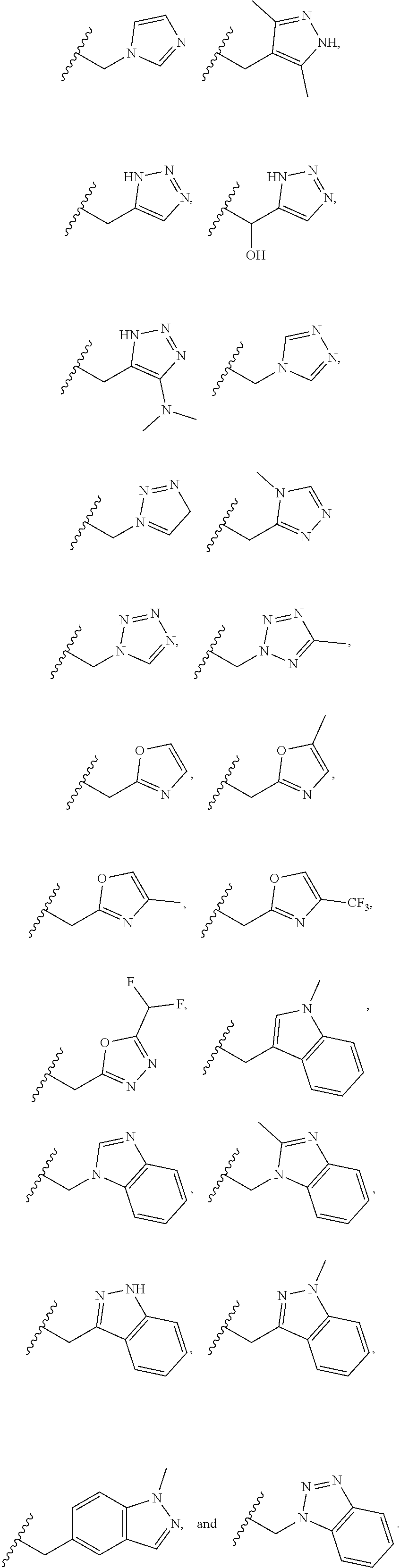

In some embodiments, R² is

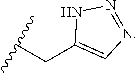 [SMILES: *Cc1cnn[nH]1]

In some embodiments of a compound of formula (I-A1), or a stereoisomer or tautomer thereof, or a pharmaceutically acceptable salt of any of the foregoing, In some embodiments, R² is methyl, wherein the methyl of R² is substituted with one or more R^{a}, wherein R^{a} is 3-8 membered heterocyclyl, wherein the 3-8 membered heterocyclyl of R^{a} is optionally substituted with one or more R^{c}, wherein R^{c} is oxo, C₁₋₆alkyl, or —C(O)—C₁₋₆alkoxy, wherein the C₁₋₆alkyl of R^{c} is optionally substituted with one or more halo, and the —C(O)—C₁₋₆alkoxy of R^{c} is optionally substituted with one or more halo. In some embodiments, R² is

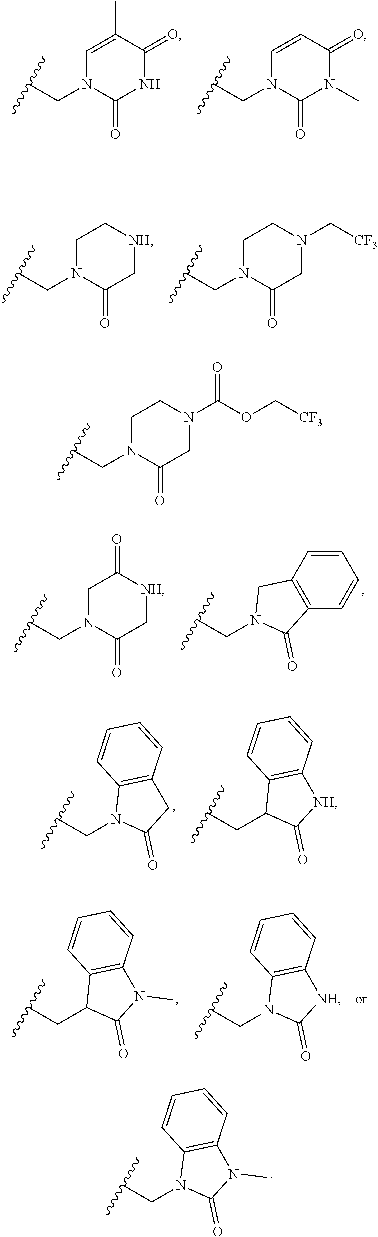

In some embodiments of a compound of formula (I-A1), or a stereoisomer or tautomer thereof, or a pharmaceutically acceptable salt of any of the foregoing, R² is methyl, wherein the methyl of R² is substituted with one or more R^{a}, wherein R^{a} is —O—R^{e}, wherein R^{e} is —C(O)-(3-15 membered heterocyclyl) wherein the —C(O)-(3-15 membered heterocyclyl) of R^{e} is optionally substituted with one or more C₁₋₆alkyl, wherein the C₁₋₆alkyl is optionally substituted with one or more halo, C₁₋₆alkoxy, or C₃₋₁₀cycloalkyl. In some embodiments, R² is

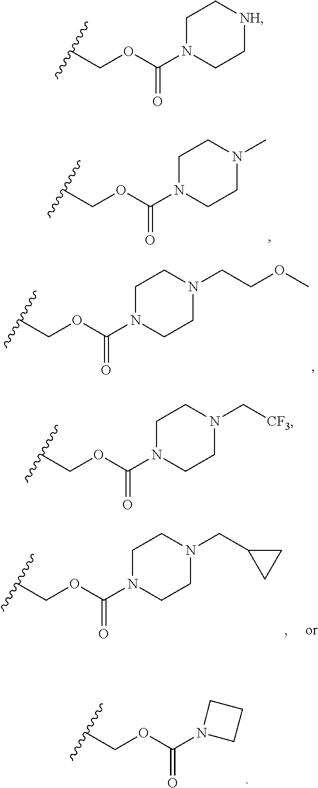

In some embodiments of a compound of formula (I-A1), or a stereoisomer or tautomer thereof, or a pharmaceutically acceptable salt of any of the foregoing, R² is methyl, wherein the methyl of R² is substituted with one or more R^{a}, wherein R^{a} is —N(R^{c})(R^{d}), wherein one of R^{c} and R^{d} is H, and the other of R^{c} and R^{d}, —C(O)—C₁₋₆alkyl, —C(O)—N(C₁₋₆alkyl)₂, or —C(O)-(3-15 membered heterocyclyl). In some embodiments, R² is

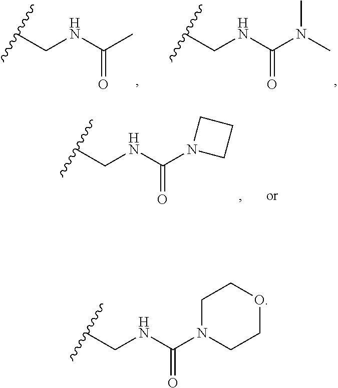

In some embodiments of a compound of formula (I-A1), or a stereoisomer or tautomer thereof, or a pharmaceutically acceptable salt of any of the foregoing, R² is ethyl, wherein the ethyl of R² is substituted with one or more R^{a}, wherein R^{a} is —N(R)(R^{d}), wherein one of R^{c} and R^{d} is H, and the other of R^{c} and R^{d}, is —C(O)—C₁₋₆alkyl. In some embodiments, R² is

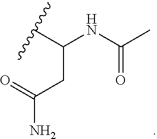 [SMILES: *C(CC(N)=O)NC(C)=O]

In some embodiments, provided is a compound of formula (I-1) or formula (I-A), or a stereoisomer or tautomer thereof, or a pharmaceutically acceptable salt of any of the foregoing, wherein the compound is a compound of formula (I-A2):

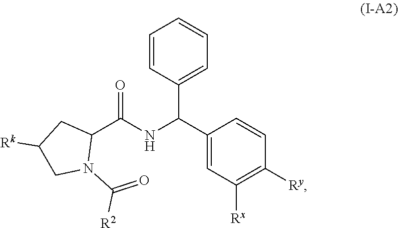

or a stereoisomer or tautomer thereof, or a pharmaceutically acceptable salt of any of the foregoing, wherein R^{x} is H, halo, C₁₋₆alkyl, or —NH₂, wherein the C₁₋₆alkyl is optionally substituted with one or more halo; and R^{y} is (i) C₁₋₆alkyl, (ii), C₂₋₆alkenyl, or (iii) C₃₋₁₀cycloalkyl, wherein the C₃₋₁₀cycloalkyl is optionally substituted with one or more halo or C₁₋₆alkyl. In some embodiments, R^{x} is H, halo, or C₁₋₆alkyl, wherein the C₁₋₆alkyl is optionally substituted with one or more halo; and R^{y} is (i), C₁₋₆alkyl, (ii) C₂₋₆alkenyl, or (iii) C₃₋₁₀cycloalkyl, wherein the C₃₋₁₀cycloalkyl is optionally substituted with one or more halo or C₁₋₆alkyl. In some embodiments, R^{x} is H, halo, or C₁₋₆alkyl; and R^{y} is (i) C₁₋₆alkyl, (ii) C₂₋₆alkenyl, or (iii) C₃₋₁₀cycloalkyl, wherein the C₃₋₁₀cycloalkyl is optionally substituted with one or more halo or C₁₋₆alkyl.

In some embodiments of a compound of formula (I-A2), or a stereoisomer or tautomer thereof, or a pharmaceutically acceptable salt of any of the foregoing, R^{x} is H, fluoro, chloro, or methyl, wherein the methyl is optionally substituted with one or more fluoro; and R^{y} is (i) isopropyl, (ii) isopropenyl, (iii) sec-butyl, (iv) tert-butyl, or (v) C₃₋₄cycloalkyl, wherein the C₃₋₄cycloalkyl is optionally substituted with one or more fluoro or methyl.

In some embodiments of a compound of formula (I-A2), or a stereoisomer or tautomer thereof, or a pharmaceutically acceptable salt of any of the foregoing, R^{x} is fluoro or methyl optionally substituted with one or more fluoro; and R^{y} is (i) isopropyl (ii) C₃₋₄cycloalkyl optionally substituted with one or more halo or C₁₋₆alkyl or (iii) butyl.

In some embodiments of a compound of formula (I-A2), or a stereoisomer or tautomer thereof, or a pharmaceutically acceptable salt of any of the foregoing, R^{k} is H or halo. In some embodiments of a compound of formula (I-A2), or a stereoisomer or tautomer thereof, or a pharmaceutically acceptable salt of any of the foregoing, R^{k} is H or fluoro. In some embodiments of a compound of formula (I-A2), or a stereoisomer or tautomer thereof, or a pharmaceutically acceptable salt of any of the foregoing, R^{k} is fluoro.

In some embodiments of a compound of formula (I-A2), or a stereoisomer or tautomer thereof, or a pharmaceutically acceptable salt of any of the foregoing, R² is C₁₋₆alkyl, wherein the C₁₋₆alkyl of R² is optionally substituted with one or more R^{a}. In some embodiments, R² is C₁₋₆alkyl, wherein the C₁₋₆alkyl of R² is substituted with one or more R^{a}, wherein R^{a} is —OH or 5-20 membered heteroaryl, wherein the 5-20 membered heteroaryl of R^{a} is optionally substituted with one or more R^{b}. In some embodiments, R² is methyl, wherein the methyl of R² is substituted with one or more R^{a}, wherein R^{a} is —OH or 5-10 membered heteroaryl, wherein the 5-10 membered heteroaryl of R^{a} is optionally substituted with one or more R^{b}. In some embodiments, R² is methyl, wherein the methyl of R² is substituted with one or more R^{a}, wherein R^{a} is —OH or 5-10 membered heteroaryl, wherein the 5-10 membered heteroaryl of R^{a} is optionally substituted with one or more C₁₋₆alkyl, wherein the C₁₋₆alkyl is optionally substituted with one or more halo, —NH₂, —NH(C₁₋₆alkyl), —N(C₁₋₆alkyl)₂, —NH—C(O)C₁₋₆alkyl, or —NH—C(O)—C₁₋₆alkoxy. In some embodiments, R² is methyl, wherein the methyl of R² is substituted with one or more R^{a}, wherein R^{a} is —OH or 5-10 membered heteroaryl, wherein the 5-10 membered heteroaryl of R^{a} is optionally substituted with one or more methyl, wherein the methyl is optionally substituted with one or more fluoro.

In some embodiments of a compound of formula (I-A2), or a stereoisomer or tautomer thereof, or a pharmaceutically acceptable salt of any of the foregoing, R² is —(C₁₋₄alkyl)(5-20 membered heteroaryl), wherein the C₁₋₄ alkyl is optionally substituted with one or more or more —OH, and wherein the 5-20 membered heteroaryl is optionally substituted with one or more R^{s}. In some embodiments, R^{s} is halo, C₁₋₆alkyl, C₁₋₆alkoxy, —NH₂, —NH(C₁₋₆alkyl), —N(C₁₋₆alkyl)₂, —NH—C(O)—C₁₋₆alkyl, C₆₋₂₀aryl, C₃₋₁₀cycloalkyl, 3-15 membered heterocyclyl, 5-20 membered heteroaryl, or —C(O)—C₁₋₆alkoxy. In some embodiments, the C₁₋₆alkyl of R^{s} is optionally substituted with one or more halo, C₁₋₆alkoxy, —NH₂, —NH(C₁₋₆alkyl), —N(C₁₋₆alkyl)₂, —NH—C(O)C₁₋₆alkyl, or —NH—C(O)—C₁₋₆alkoxy, and the 3-15-membered heterocyclyl of R^{s} is optionally substituted with one or more halo or —C(O)—C₁₋₆alkoxy.

In some embodiments of a compound of formula (I-A2), or a stereoisomer or tautomer thereof, or a pharmaceutically acceptable salt of any of the foregoing, R² is selected from the group consisting of

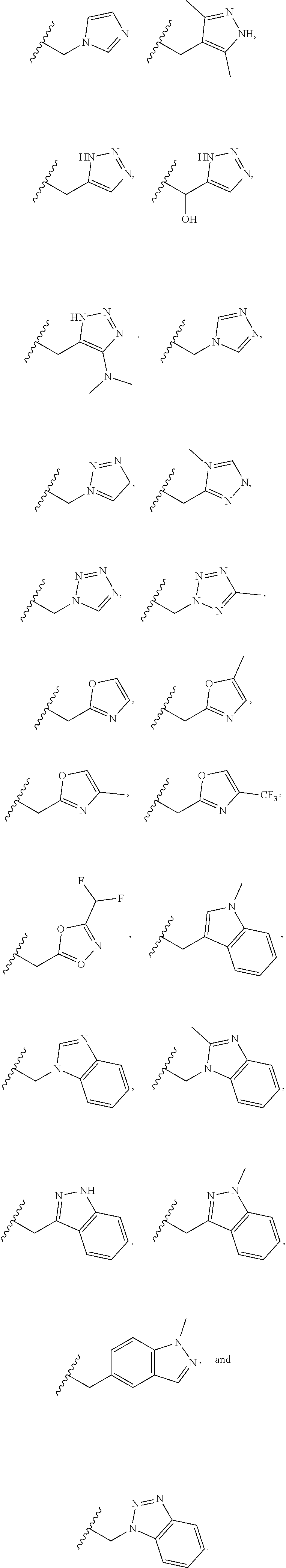

In some embodiments, R² is

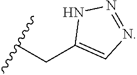 [SMILES: *Cc1cnn[nH]1]

In some embodiments of a compound of formula (I-A2), or a stereoisomer or tautomer thereof, or a pharmaceutically acceptable salt of any of the foregoing, R² is methyl, wherein the methyl of R² is substituted with one or more R^{a}, wherein R^{a} is 3-8 membered heterocyclyl, wherein the 3-8 membered heterocyclyl of R^{a} is optionally substituted with one or more R^{c}, wherein R^{c} is oxo, C₁₋₆alkyl, or —C(O)—C₁₋₆alkoxy, wherein the C₁₋₆alkyl of R^{c} is optionally substituted with one or more halo, and the —C(O)—C₁₋₆alkoxy of R^{c} is optionally substituted with one or more halo. In some embodiments, R² is selected from the group consisting of

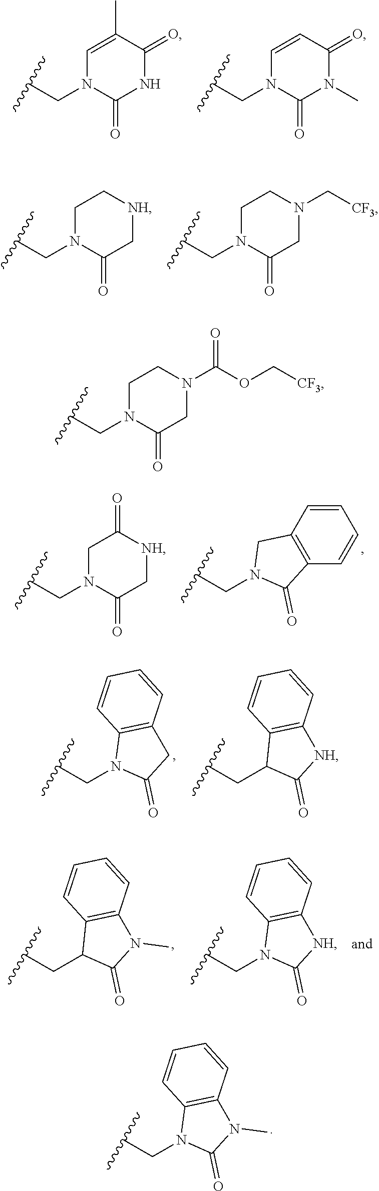

In some embodiments of a compound of formula (I-A2), or a stereoisomer or tautomer thereof, or a pharmaceutically acceptable salt of any of the foregoing, R² is methyl, wherein the methyl of R² is substituted with one or more R^{a}, wherein R^{a} is —O—R^{e}, wherein R^{e} is —C(O)-(3-15 membered heterocyclyl) wherein the —C(O)-(3-15 membered heterocyclyl) of R^{e} is optionally substituted with one or more C₁₋₆alkyl, wherein the C₁₋₆alkyl is optionally substituted with one or more halo, C₁₋₆alkoxy, or C₃₋₁₀cycloalkyl. In some embodiments, R² is selected from the group consisting of

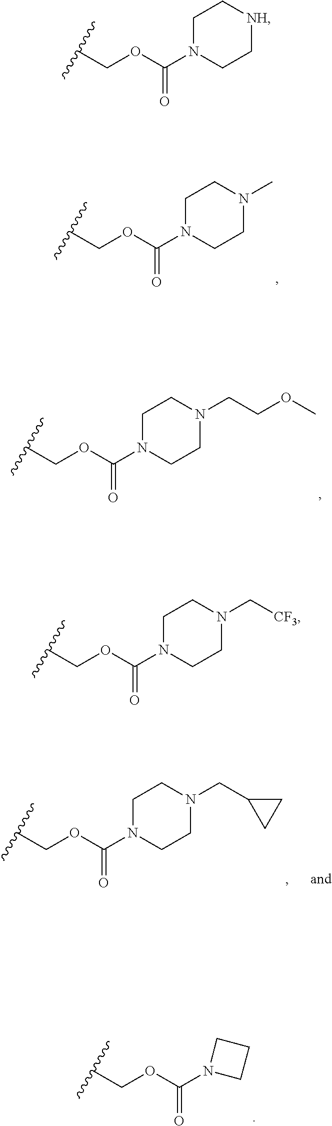 [SMILES: *COC(=O)N1CCNCC1 ; *COC(=O)N1CCN(C)CC1 ; *COC(=O)N1CCN(CCOC)CC1 ; *COC(=O)N1CCN(CC(F)(F)F)CC1 ; *COC(=O)N1CCN(CC2CC2)CC1 ; *COC(=O)N1CCC1]

In some embodiments of a compound of formula (I-A2), or a stereoisomer or tautomer thereof, or a pharmaceutically acceptable salt of any of the foregoing, R² is methyl, wherein the methyl of R² is substituted with one or more R^{a}, wherein R^{a} is —N(R^{c})(R^{d}), wherein one of R^{c} and R^{d} is H, and the other of R^{c} and R^{d}, —C(O)—C₁₋₆alkyl, —C(O)—N(C₁₋₆alkyl)₂, or —C(O)-(3-15 membered heterocyclyl). In some embodiments, R² is selected from the group consisting of

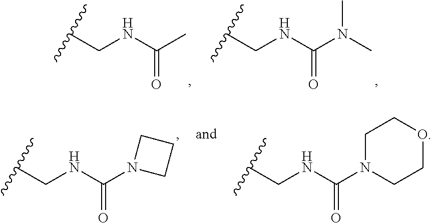

In some embodiments of a compound of formula (I-A2), or a stereoisomer or tautomer thereof, or a pharmaceutically acceptable salt of any of the foregoing, R² is ethyl, wherein the ethyl of R² is substituted with one or more R^{a}, wherein R^{a} is —N(R)(R^{d}), wherein one of R^{c} and R^{d} is H, and the other of R^{c} and R^{d}, is —C(O)—C₁₋₆alkyl. In some embodiments, R² is

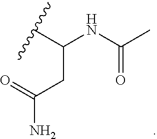 [SMILES: *C(CC(N)=O)NC(C)=O]

In some embodiments, provided herein is a compound of formula (I-1) or formula (I-A), or a stereoisomer or tautomer thereof, or a pharmaceutically acceptable salt of any of the foregoing, wherein the compound is a compound of formula (I-A3):

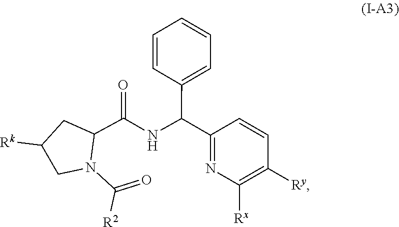

or a stereoisomer or tautomer thereof, or a pharmaceutically acceptable salt of any of the foregoing, wherein R^{x} is H, halo, C₁₋₆alkyl, or —NH₂, wherein the C₁₋₆alkyl is optionally substituted with one or more halo; and R^{y} is (i) C₁₋₆alkyl, (ii), C₂₋₆alkenyl, or (iii) C₃₋₁₀cycloalkyl, wherein the C₃₋₁₀cycloalkyl is optionally substituted with one or more halo or C₁₋₆alkyl. In some embodiments, R^{x} is H, halo, C₁₋₆alkyl, or —NH₂; and R^{y} is (i) C₁₋₆alkyl or (ii) C₃₋₁₀cycloalkyl, wherein the C₃₋₁₀cycloalkyl is optionally substituted with one or more halo or C₁₋₆alkyl.

In some embodiments of a compound of formula (I-A3), or a stereoisomer or tautomer thereof, or a pharmaceutically acceptable salt of any of the foregoing, R^{x} is H, fluoro, or methyl; and R^{y} is (i) H, (ii) isopropyl, (iii) tert-butyl, or (iv) C₃₋₄cycloalkyl, wherein the C₃₋₄cycloalkyl is optionally substituted with one or more fluoro or methyl. In some embodiments, R^{x} is H, fluoro, or methyl; and R^{y} is (i) isopropyl or (ii) C₃₋₄cycloalkyl, wherein the C₃₋₄cycloalkyl is optionally substituted with one or more fluoro or methyl.

In some embodiments of a compound of formula (I-A3), or a stereoisomer or tautomer thereof, or a pharmaceutically acceptable salt of any of the foregoing, R^{k} is H or halo. In some embodiments of a compound of formula (I-A3), or a stereoisomer or tautomer thereof, or a pharmaceutically acceptable salt of any of the foregoing, R^{k} is H or fluoro. In some embodiments of a compound of formula (I-A3), or a stereoisomer or tautomer thereof, or a pharmaceutically acceptable salt of any of the foregoing, R^{k} is fluoro.

In some embodiments of a compound of formula (I-A3), or a stereoisomer or tautomer thereof, or a pharmaceutically acceptable salt of any of the foregoing, R² is C₁₋₆alkyl, wherein the C₁₋₆alkyl of R² is optionally substituted with one or more R^{a}. In some embodiments, R² is C₁₋₆alkyl, wherein the C₁₋₆alkyl of R² is substituted with one or more R^{a}, wherein R^{a} is —OH or 5-20 membered heteroaryl, wherein the 5-20 membered heteroaryl of R^{a} is optionally substituted with one or more R^{b}. In some embodiments, R² is methyl, wherein the methyl of R² is substituted with one or more R^{a}, wherein R^{a} is —OH or 5-10 membered heteroaryl, wherein the 5-10 membered heteroaryl of R^{a} is optionally substituted with one or more R^{b}. In some embodiments, R² is methyl, wherein the methyl of R² is substituted with one or more R^{a}, wherein R^{a} is —OH or 5-10 membered heteroaryl, wherein the 5-10 membered heteroaryl of R^{a} is optionally substituted with one or more C₁₋₆alkyl, wherein the C₁₋₆alkyl is optionally substituted with one or more halo, —NH₂, —NH(C₁₋₆alkyl), —N(C₁₋₆alkyl)₂, —NH—C(O)C₁₋₆alkyl, or —NH—C(O)—C₁₋₆alkoxy. In some embodiments, R² is methyl, wherein the methyl of R² is substituted with one or more R^{a}, wherein R^{a} is —OH or 5-10 membered heteroaryl, wherein the 5-10 membered heteroaryl of R^{a} is optionally substituted with one or more methyl, wherein the methyl is optionally substituted with one or more fluoro.

In some embodiments of a compound of formula (I-A3), or a stereoisomer or tautomer thereof, or a pharmaceutically acceptable salt of any of the foregoing, R² is —(C₁₋₄alkyl)(5-20 membered heteroaryl), wherein the 5-20 membered heteroaryl is optionally substituted with one or more R^{s}. In some embodiments, R^{s} is halo, C₁₋₆alkyl, C₁₋₆alkoxy, —NH₂, —NH(C₁₋₆alkyl), —N(C₁₋₆alkyl)₂, —NH—C(O)—C₁₋₆alkyl, C₆₋₂₀aryl, C₃₋₁₀cycloalkyl, 3-15 membered heterocyclyl, 5-20 membered heteroaryl, or —C(O)—C₁₋₆alkoxy. In some embodiments, the C₁₋₆alkyl of R^{s} is optionally substituted with one or more halo, C₁₋₆alkoxy, —NH₂, —NH(C₁₋₆alkyl), —N(C₁₋₆alkyl)₂, —NH—C(O)C₁₋₆alkyl, or —NH—C(O)—C₁₋₆alkoxy, and the 3-15-membered heterocyclyl of R^{s} is optionally substituted with one or more halo or —C(O)—C₁₋₆alkoxy.

In some embodiments of a compound of formula (I-A3), or a stereoisomer or tautomer thereof, or a pharmaceutically acceptable salt of any of the foregoing, R² is selected from the group consisting of

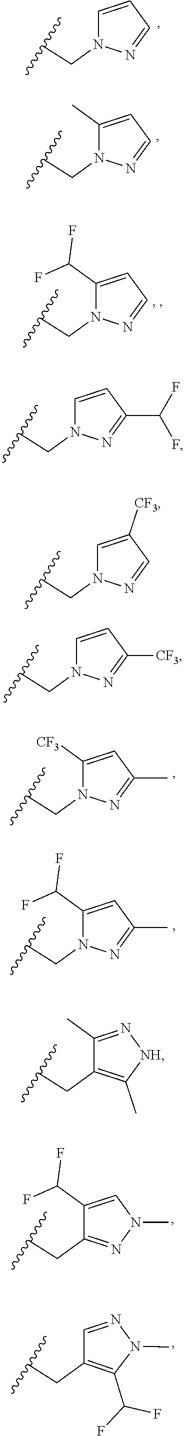

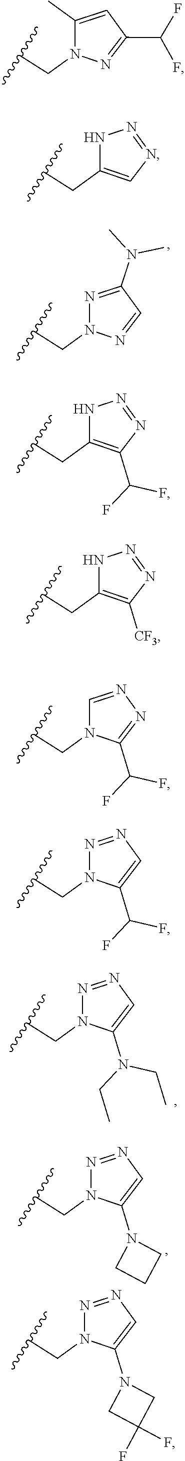

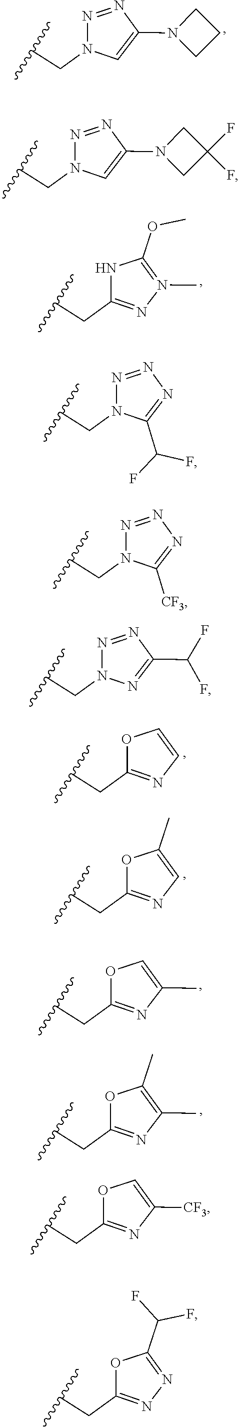

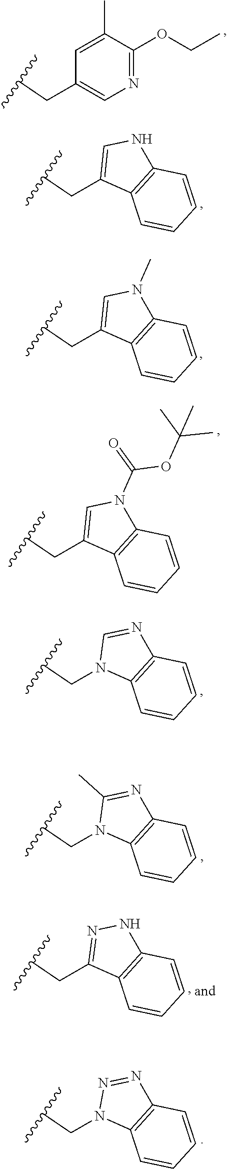

In some embodiments, R² is

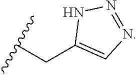 [SMILES: *Cc1cnn[nH]1]

In some embodiments, R² is

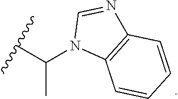 [SMILES: *C(C)n1cnc2ccccc21]

In some embodiments of a compound of formula (I-A3), or a stereoisomer or tautomer thereof, or a pharmaceutically acceptable salt of any of the foregoing, In some embodiments, R² is methyl, wherein the methyl of R² is substituted with one or more R^{a}, wherein R^{a} is 3-8 membered heterocyclyl, wherein the 3-8 membered heterocyclyl of R^{a} is optionally substituted with one or more R^{c}, wherein R^{c} is oxo, C₁₋₆alkyl, or —C(O)—C₁₋₆alkoxy, wherein the C₁₋₆alkyl of R^{c} is optionally substituted with one or more halo, and the —C(O)—C₁₋₆alkoxy of R^{c} is optionally substituted with one or more halo. In some embodiments, R² is

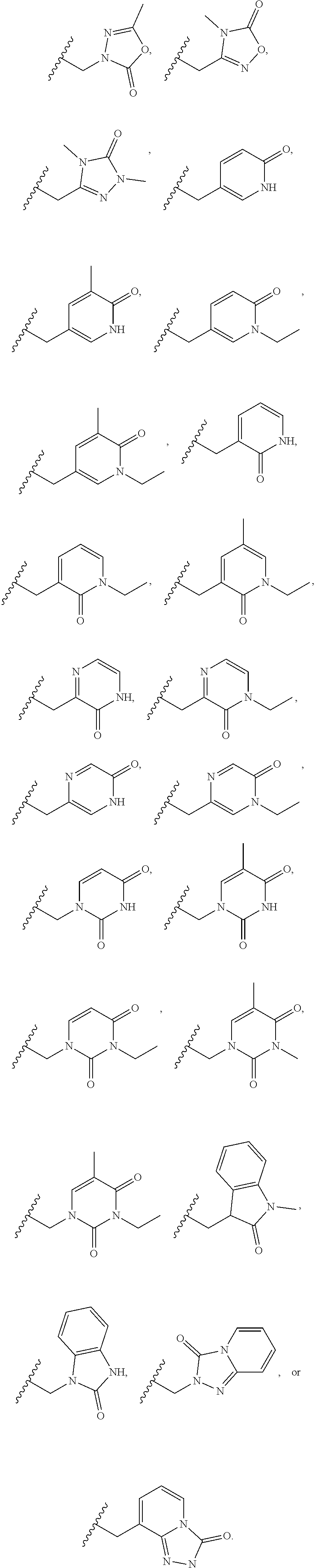

In some embodiments of a compound of formula (I-A3), or a stereoisomer or tautomer thereof, or a pharmaceutically acceptable salt of any of the foregoing, R² is C₁₋₆alkyl, wherein the C₁₋₆alkyl of R² is substituted with one or more R^{a}, wherein R^{a} is —O—R^{e}. In some embodiments, R² is methyl, wherein the methyl of R² is substituted with one or more R^{a}, wherein R^{a} is —O—R^{e}. In some embodiments, R² is methyl, wherein the methyl of R² is substituted with one or more R^{a}, wherein R^{a} is —O—R^{e}, wherein R^{e} is —C(O)-(3-15 membered heterocyclyl). In some embodiments, R² is

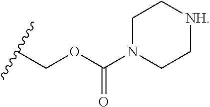 [SMILES: *COC(=O)N1CCNCC1]

In some embodiments of a compound of formula (I-A3), or a stereoisomer or tautomer thereof, or a pharmaceutically acceptable salt of any of the foregoing, R² is methyl, wherein the methyl of R² is substituted with one or more R^{a}, wherein R^{a} is —N(R^{c})(R^{d}), wherein one of R^{c} and R^{d} is H, and the other of R^{c} and R^{d}, —C(O)—C₁₋₆alkyl, —C(O)—N(C₁₋₆alkyl)₂, or —C(O)-(3-15 membered heterocyclyl), wherein the —C(O)-(3-15 membered heterocyclyl) of R^{c} or R^{d} is optionally substituted with one or more halo, —C(O)—C₁₋₆alkoxy, or C₁₋₆alkyl, and the C₁₋₆alkyl of the —C(O)—N(C₁₋₆alkyl)₂ of R^{c} or R^{d} are, independently of each other, optionally substituted with one or more halo or C₆₋₂₀aryl. In some embodiments, R² is

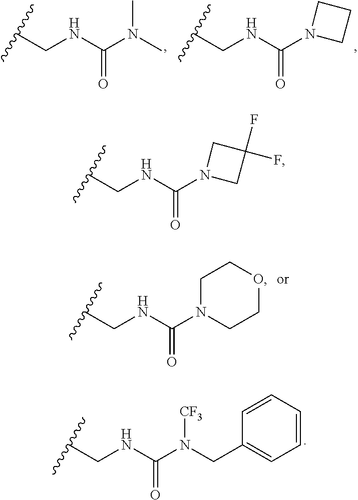

In some embodiments of a compound of formula (I-A3), or a stereoisomer or tautomer thereof, or a pharmaceutically acceptable salt of any of the foregoing, R² is methyl, wherein the methyl of R² is substituted with one or more R^{a}, wherein R^{a} is —N(R^{c})(R^{d}), wherein one of R^{c} and R^{d} is H, and the other of R^{c} and R^{d} is 5-20 membered heteroaryl, wherein the 5-20 membered heteroaryl of R^{c} or R^{d} are independently optionally substituted with one or more C₁₋₆alkyl. In some embodiments, R² is

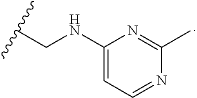 [SMILES: *CNc1ccnc(C)n1]

In some embodiments, provided herein is a compound of formula (I-1) or formula (I-A), or a stereoisomer or tautomer thereof, or a pharmaceutically acceptable salt of any of the foregoing, wherein the compound is a compound of formula (I-A4):

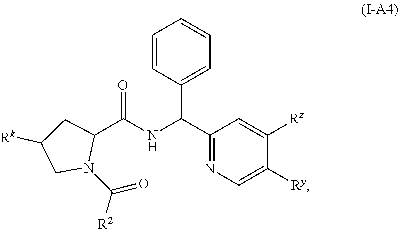

or a stereoisomer or tautomer thereof, or a pharmaceutically acceptable salt of any of the foregoing, wherein R^{z} is H, halo, C₁₋₆alkyl, or —NH₂ and R^{y} is (i) C₁₋₆alkyl, (ii), C₂₋₆alkenyl, or (iii) C₃₋₁₀cycloalkyl, wherein the C₃₋₁₀cycloalkyl is optionally substituted with one or more halo or C₁₋₆alkyl. In some embodiments, R^{z} is H, halo, or C₁₋₆alkyl; and R^{y} is (i) C₁₋₆alkyl, (ii), C₂₋₆alkenyl, or (iii) C₃₋₁₀cycloalkyl, wherein the C₃₋₁₀cycloalkyl is optionally substituted with one or more halo or C₁₋₆alkyl. In some embodiments, R^{z} is H or C₁₋₆alkyl; and R^{y} is (i) C₁₋₆alkyl or (ii) C₃₋₁₀cycloalkyl, wherein the C₃₋₁₀cycloalkyl is optionally substituted with one or more halo or C₁₋₆alkyl. In some embodiments of a compound of formula (I-A4), or a stereoisomer or tautomer thereof, or a pharmaceutically acceptable salt of any of the foregoing, R^{z} is H or methyl; and R^{y} is (i) isopropyl, or (ii) C₃₋₄cycloalkyl.

In some embodiments of a compound of formula (I-A4), or a stereoisomer or tautomer thereof, or a pharmaceutically acceptable salt of any of the foregoing, R^{k} is H or halo. In some embodiments of a compound of formula (I-A4), or a stereoisomer or tautomer thereof, or a pharmaceutically acceptable salt of any of the foregoing, R^{k} is H or fluoro. In some embodiments of a compound of formula (I-A4), or a stereoisomer or tautomer thereof, or a pharmaceutically acceptable salt of any of the foregoing, R^{k} is fluoro.

In some embodiments of a compound of formula (I-A4), or a stereoisomer or tautomer thereof, or a pharmaceutically acceptable salt of any of the foregoing, R² is C₁₋₆alkyl, wherein the C₁₋₆alkyl of R² is optionally substituted with one or more R^{a}. In some embodiments, R² is C₁₋₆alkyl, wherein the C₁₋₆alkyl of R² is substituted with one or more R^{a}, wherein R^{a} is —OH or 5-20 membered heteroaryl, wherein the 5-20 membered heteroaryl of R^{a} is optionally substituted with one or more. R^{b} In some embodiments, R² is methyl, wherein the methyl of R² is substituted with one or more R^{a}, wherein R^{a} is —OH or 5-10 membered heteroaryl, wherein the 5-10 membered heteroaryl of R^{a} is optionally substituted with one or more R^{b}. In some embodiments, R² is methyl, wherein the methyl of R² is substituted with one or more R^{a}, wherein R^{a} is —OH or 5-10 membered heteroaryl, wherein the 5-10 membered heteroaryl of R^{a} is optionally substituted with one or more C₁₋₆alkyl, wherein the C₁₋₆alkyl is optionally substituted with one or more halo, —NH₂, —NH(C₁₋₆alkyl), —N(C₁₋₆alkyl)₂, —NH—C(O)C₁₋₆alkyl, or —NH—C(O)—C₁₋₆alkoxy. In some embodiments, R² is methyl, wherein the methyl of R² is substituted with one or more R^{a}, wherein R^{a} is —OH or 5-10 membered heteroaryl, wherein the 5-10 membered heteroaryl of R^{a} is optionally substituted with one or more methyl, wherein the methyl is optionally substituted with one or more fluoro.

In some embodiments of a compound of formula (I-A4), or a stereoisomer or tautomer thereof, or a pharmaceutically acceptable salt of any of the foregoing, R² is selected from the group consisting of

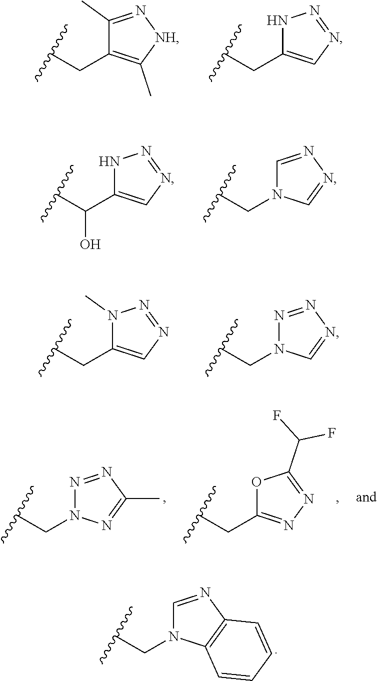

In some embodiments, R² is

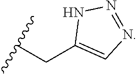 [SMILES: *Cc1cnn[nH]1]

In some embodiments of a compound of formula (I-A4), or a stereoisomer or tautomer thereof, or a pharmaceutically acceptable salt of any of the foregoing, R² is C₁₋₆alkyl, wherein the C₁₋₆alkyl of R² is substituted with one or more R^{a}, wherein R^{a} is 3-15 membered heterocyclyl, wherein the 3-15 membered heterocyclyl of R^{a} is optionally substituted with one or more R^{c}. In some embodiments, R² is methyl, wherein the methyl of R² is substituted with one or more R^{a}, wherein R^{a} is 3-15 membered heterocyclyl, wherein the 3-15 membered heterocyclyl of R^{a} is optionally substituted with one or more R^{c}. In some embodiments, R² is methyl, wherein the methyl of R² is substituted with one or more R^{a}, wherein R^{a} is 3-8 membered heterocyclyl, wherein the 3-8 membered heterocyclyl of R^{a} is optionally substituted with one or more R^{c}. In some embodiments, R² is methyl, wherein the methyl of R² is substituted with one or more R^{a}, wherein R^{a} is 3-8 membered heterocyclyl, wherein the 3-8 membered heterocyclyl of R^{a} is optionally substituted with one or more oxo or C₁₋₆alkyl. In some embodiments, R² is

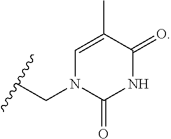 [SMILES: *Cn1cc(C)c(=O)[nH]c1=O]

In some embodiments of a compound of formula (I-A4), or a stereoisomer or tautomer thereof, or a pharmaceutically acceptable salt of any of the foregoing, R² is C₁₋₆alkyl, wherein the C₁₋₆alkyl of R² is substituted with one or more R^{a}, wherein R^{a} is —O—R^{e}. In some embodiments, R² is methyl, wherein the methyl of R² is substituted with one or more R^{a}, wherein R^{a} is —O—R^{e}. In some embodiments, R² is methyl, wherein the methyl of R² is substituted with one or more R^{a}, wherein R^{a} is —O—R^{e}, wherein R^{e} is —C(O)-(3-15 membered heterocyclyl). In some embodiments, R² is

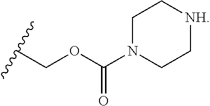 [SMILES: *COC(=O)N1CCNCC1]

In some embodiments of a compound of formula (I-A4), or a stereoisomer or tautomer thereof, or a pharmaceutically acceptable salt of any of the foregoing, R² is C₁₋₆alkyl, wherein the C₁₋₆alkyl of R² is substituted with one or more R^{a}, wherein R^{a} is —N(R^{c})(R^{d}). In some embodiments, R² is methyl, wherein the methyl of R² is substituted with one or more R^{a}, wherein R^{a} is —N(R^{c})(R^{d}). In some embodiments, R² is methyl, wherein the methyl of R² is substituted with one or more R^{a}, wherein R^{a} is —N(R^{c})(R^{d}), wherein one of R^{c} and R^{d} is H, and the other of R^{c} and R^{d} is —C(O)—N(C₁₋₆alkyl)₂. In some embodiments, R² is

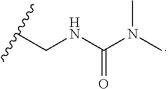 [SMILES: *CNC(=O)N(C)C]

In some embodiments, provided herein is a compound of formula (I-1), or a stereoisomer or tautomer thereof, or a pharmaceutically acceptable salt of any of the foregoing, wherein the compound is a compound of formula (I-B):

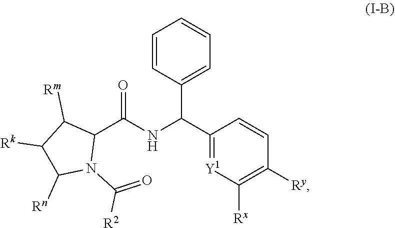

or a stereoisomer or tautomer thereof, or a pharmaceutically acceptable salt of any of the foregoing, or any variation or embodiment thereof, wherein Y¹ is CR^{x} or N; wherein, when the ring bearing R^{x}, and R^{y} is phenyl, R^{x}, and R^{y} are each independently H, halo, C₁₋₆alkyl, C₂₋₆alkenyl, —NH₂, —NH—C(O)—(C₁₋₆alkyl), —NH—C(O)-(3-15 membered heterocyclyl), C₃₋₁₀cycloalkyl, or 5-20 membered heteroaryl, wherein the C₁₋₆alkyl is optionally substituted with one or more halo, —NH—C(O)—NH(C₁₋₆alkyl), —NH—C(O)—C₁₋₆alkyl, or —NH—C(O)—C₁₋₆alkoxy, and the C₃₋₁₀cycloalkyl is optionally substituted with one or more halo or C₁₋₆alkyl, and the 5-20 membered heteroaryl is optionally substituted with one or more C₁₋₆alkyl; and wherein when the ring bearing R^{x}, and R^{y} is pyridyl, R^{x}, and R^{y} are each independently H, halo, C₁₋₆alkyl, C₂₋₆alkenyl, C₁₋₆alkoxy, —NH₂, or C₃₋₁₀cycloalkyl, wherein, the C₁₋₆alkyl is optionally substituted with one or more halo, and the C₃₋₁₀cycloalkyl is optionally substituted with one or more halo or C₁₋₆alkyl.

In some embodiments of a compound of formula (I-B), or a stereoisomer or tautomer thereof, or a pharmaceutically acceptable salt of any of the foregoing, Y¹ is CH or N; R^{x} is H, halo, C₁₋₆alkyl, or NH₂, wherein, when Y¹ is CH, the C₁₋₆alkyl of R^{x} may be optionally substituted with one or more halo; and R^{y} is (i) C₁₋₆alkyl, (ii), C₂₋₆alkenyl, or (iii) C₃₋₁₀cycloalkyl, wherein the C₃₋₁₀cycloalkyl is optionally substituted with one or more halo or C₁₋₆alkyl. In some embodiments, Y¹ is CH or N; R^{x} is H or halo; R^{y} is C₁₋₆alkyl or C₃₋₁₀cycloalkyl; and R^{k} is taken together with either R^{m} or Rⁿ, and the atoms to which they are attached, to form cyclopropyl. In some embodiments, Y¹ is CH or N; R^{x} is H or fluoro; R^{y} is (i) isopropyl or (ii) C₃₋₄cycloalkyl; and R^{k} is taken together with either R^{m} or Rⁿ, and the atoms to which they are attached, to form cyclopropyl. In some embodiments, Y¹ is CH or N; R^{x} is H or fluoro; R^{y} is (i) isopropyl or (ii) C₃₋₄cycloalkyl; and R^{k} is taken together with R^{m} and the atoms to which they are attached to form cyclopropyl. In some embodiments, Y¹ is CH or N; R^{x} is H or fluoro; R^{y} is (i) isopropyl or (ii) C₃₋₄cycloalkyl; and R^{k} is taken together with Rⁿ and the atoms to which they are attached to form cyclopropyl. In some embodiments, Y¹ is CH; R^{x} is H or fluoro; R^{y} is (i) isopropyl or (ii) C₃₋₄cycloalkyl; and R^{k} is taken together with Rⁿ or Rⁿ and the atoms to which they are attached to form cyclopropyl. In some embodiments, Y¹ is N; R^{x} is H or fluoro; R^{y} is (i) isopropyl or (ii) C₃₋₄cycloalkyl; and R^{k} is taken together with Rⁿ or Rⁿ and the atoms to which they are attached to form cyclopropyl.

In some embodiments of a compound of formula (I-B), or a stereoisomer or tautomer thereof, or a pharmaceutically acceptable salt of any of the foregoing, R² is C₁₋₆alkyl, wherein the C₁₋₆alkyl of R² is optionally substituted with one or more R^{a}. In some embodiments, R² is C₁₋₆alkyl, wherein the C₁₋₆alkyl of R² is substituted with one or more R^{a}, wherein R^{a} is —OH or 5-20 membered heteroaryl, wherein the 5-20 membered heteroaryl of R^{a} is optionally substituted with one or more R^{b}. In some embodiments, R² is methyl, wherein the methyl of R² is substituted with one or more R^{a}, wherein R^{a} is —OH or 5-10 membered heteroaryl, wherein the 5-10 membered heteroaryl of R^{a} is optionally substituted with one or more R^{b}. In some embodiments, R² is methyl, wherein the methyl of R² is substituted with one or more R^{a}, wherein R^{a} is —OH or 5-10 membered heteroaryl, wherein the 5-10 membered heteroaryl of R^{a} is optionally substituted with one or more C₁₋₆alkyl, wherein the C₁₋₆alkyl is optionally substituted with one or more halo, —NH₂, —NH(C₁₋₆alkyl), —N(C₁₋₆alkyl)₂, —NH—C(O)C₁₋₆alkyl, or —NH—C(O)—C₁₋₆alkoxy. In some embodiments, R² is methyl, wherein the methyl of R² is substituted with one or more R^{a}, wherein R^{a} is —OH or 5-10 membered heteroaryl, wherein the 5-10 membered heteroaryl of R^{a} is optionally substituted with one or more methyl, wherein the methyl is optionally substituted with one or more fluoro.

In some embodiments of a compound of formula (I-B), or a stereoisomer or tautomer thereof, or a pharmaceutically acceptable salt of any of the foregoing, R² is selected from the group consisting of

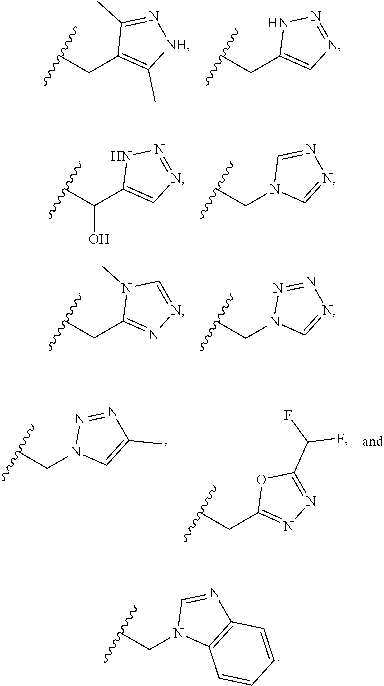

In some embodiments, R² is

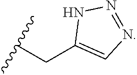 [SMILES: *Cc1cnn[nH]1]

In some embodiments of a compound of formula (I-B), or a stereoisomer or tautomer thereof, or a pharmaceutically acceptable salt of any of the foregoing, R² is C₁₋₆alkyl, wherein the C₁₋₆alkyl of R² is substituted with one or more R^{a}, wherein R^{a} is 3-15 membered heterocyclyl, wherein the 3-15 membered heterocyclyl of R^{a} is optionally substituted with one or more R^{c}. In some embodiments, R² is methyl, wherein the methyl of R² is substituted with one or more R^{a}, wherein R^{a} is 3-15 membered heterocyclyl, wherein the 3-15 membered heterocyclyl of R^{a} is optionally substituted with one or more R^{c}. In some embodiments, R² is methyl, wherein the methyl of R² is substituted with one or more R^{a}, wherein R^{a} is 3-8 membered heterocyclyl, wherein the 3-8 membered heterocyclyl of R^{a} is optionally substituted with one or more R^{c}. In some embodiments, R² is methyl, wherein the methyl of R² is substituted with one or more R^{a}, wherein R^{a} is 3-8 membered heterocyclyl, wherein the 3-8 membered heterocyclyl of R^{a} is optionally substituted with one or more oxo or C₁₋₆alkyl. In some embodiments, R² is

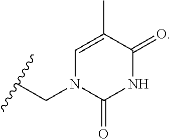 [SMILES: *Cn1cc(C)c(=O)[nH]c1=O]

In some embodiments of a compound of formula (I-B), or a stereoisomer or tautomer thereof, or a pharmaceutically acceptable salt of any of the foregoing, R² is C₁₋₆alkyl, wherein the C₁₋₆alkyl of R² is substituted with one or more R^{a}, wherein R^{a} is —O—R^{e}. In some embodiments, R² is methyl, wherein the methyl of R² is substituted with one or more R^{a}, wherein R^{a} is —O—R^{e}. In some embodiments, R² is methyl, wherein the methyl of R² is substituted with one or more R^{a}, wherein R^{a} is —O—R^{e}, wherein R^{e} is —C(O)-(3-15 membered heterocyclyl). In some embodiments, R² is

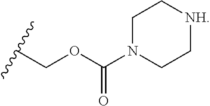 [SMILES: *COC(=O)N1CCNCC1]

In some embodiments of a compound of formula (I-B), or a stereoisomer or tautomer thereof, or a pharmaceutically acceptable salt of any of the foregoing, R² is C₁₋₆alkyl, wherein the C₁₋₆alkyl of R² is substituted with one or more R^{a}, wherein R^{a} is —N(R^{c})(R^{d}). In some embodiments, R² is methyl, wherein the methyl of R² is substituted with one or more R^{a}, wherein R^{a} is —N(R^{c})(R^{d}). In some embodiments, R² is methyl, wherein the methyl of R² is substituted with one or more R^{a}, wherein R^{a} is —N(R^{c})(R^{d}), wherein one of R^{c} and R^{d} is H, and the other of R^{c} and R^{d} is —C(O)—N(C₁₋₆alkyl)₂. In some embodiments, R² is

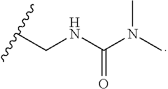 [SMILES: *CNC(=O)N(C)C]

In some embodiments, provided here is a compound of formula (I-1) or formula (I-B), or a stereoisomer or tautomer thereof, or a pharmaceutically acceptable salt of any of the foregoing, wherein the compound is a compound of formula (I-B1):

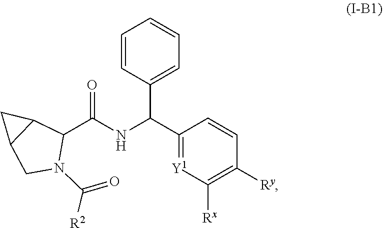

or a stereoisomer or tautomer thereof, or a pharmaceutically acceptable salt of any of the foregoing, or any variation or embodiment thereof, wherein Y¹ is CR^{x} or N; wherein, when the ring bearing R^{x}, and R^{y} is phenyl, R^{x}, and R^{y} are each independently H, halo, C₁₋₆alkyl, C₂₋₆alkenyl, —NH₂, —NH—C(O)—(C₁₋₆alkyl), —NH—C(O)-(3-15 membered heterocyclyl), C₃₋₁₀cycloalkyl, or 5-20 membered heteroaryl, wherein the C₁₋₆alkyl is optionally substituted with one or more halo, —NH—C(O)—NH(C₁₋₆alkyl), —NH—C(O)—C₁₋₆alkyl, or —NH—C(O)—C₁₋₆alkoxy, the C₃₋₁₀cycloalkyl is optionally substituted with one or more halo or C₁₋₆alkyl, and the 5-20 membered heteroaryl is optionally substituted with one or more C₁₋₆alkyl; and wherein when the ring bearing R^{x}, and R^{y} is pyridyl, R^{x}, and R^{y} are each independently H, halo, C₁₋₆alkyl, C₂₋₆alkenyl, C₁₋₆alkoxy, —NH₂, or C₃₋₁₀cycloalkyl, wherein, the C₁₋₆alkyl is optionally substituted with one or more halo, and the C₃₋₁₀cycloalkyl is optionally substituted with one or more halo or C₁₋₆alkyl. In some embodiments, Y¹ is CH or N; R^{x} is H or fluoro; and R^{y} is (i) isopropyl or (ii) C₃₋₄cycloalkyl. In some embodiments, Y¹ is CH; R^{x} is H or fluoro; and R^{y} is (i) isopropyl or (ii) C₃₋₄cycloalkyl. In some embodiments, Y¹ is N; R^{x} is H or fluoro; and R^{y} is (i) isopropyl or (ii) C₃₋₄cycloalkyl.

In some embodiments of a compound of formula (I-B1), or a stereoisomer or tautomer thereof, or a pharmaceutically acceptable salt of any of the foregoing, R² is C₁₋₆alkyl, wherein the C₁₋₆alkyl of R² is optionally substituted with one or more R^{a}. In some embodiments, R² is C₁₋₆alkyl, wherein the C₁₋₆alkyl of R² is substituted with one or more R^{a}, wherein R^{a} is —OH or 5-20 membered heteroaryl, wherein the 5-20 membered heteroaryl of R^{a} is optionally substituted with one or more R^{b} In some embodiments, R² is methyl, wherein the methyl of R² is substituted with one or more R^{a}, wherein R^{a} is —OH or 5-10 membered heteroaryl, wherein the 5-10 membered heteroaryl of R^{a} is optionally substituted with one or more R^{b}. In some embodiments, R² is methyl, wherein the methyl of R² is substituted with one or more R^{a}, wherein R^{a} is —OH or 5-10 membered heteroaryl, wherein the 5-10 membered heteroaryl of R^{a} is optionally substituted with one or more C₁₋₆alkyl, wherein the C₁₋₆alkyl is optionally substituted with one or more halo, —NH₂, —NH(C₁₋₆alkyl), —N(C₁₋₆alkyl)₂, —NH—C(O)C₁₋₆alkyl, or —NH—C(O)—C₁₋₆alkoxy. In some embodiments, R² is methyl, wherein the methyl of R² is substituted with one or more R^{a}, wherein R^{a} is —OH or 5-10 membered heteroaryl, wherein the 5-10 membered heteroaryl of R^{a} is optionally substituted with one or more methyl, wherein the methyl is optionally substituted with one or more fluoro.

In some embodiments of a compound of formula (I-B1), or a stereoisomer or tautomer thereof, or a pharmaceutically acceptable salt of any of the foregoing, R² is selected from the group consisting of

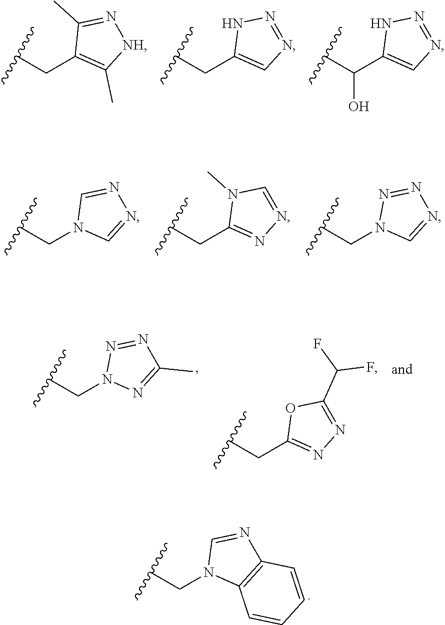

In some embodiments, R² is

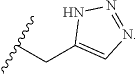 [SMILES: *Cc1cnn[nH]1]

In some embodiments of a compound of formula (I-B1), or a stereoisomer or tautomer thereof, or a pharmaceutically acceptable salt of any of the foregoing, R² is C₁₋₆alkyl, wherein the C₁₋₆alkyl of R² is substituted with one or more R^{a}, wherein R^{a} is 3-15 membered heterocyclyl, wherein the 3-15 membered heterocyclyl of R^{a} is optionally substituted with one or more R^{c}. In some embodiments, R² is methyl, wherein the methyl of R² is substituted with one or more R^{a}, wherein R^{a} is 3-15 membered heterocyclyl, wherein the 3-15 membered heterocyclyl of R^{a} is optionally substituted with one or more R^{c}. In some embodiments, R² is methyl, wherein the methyl of R² is substituted with one or more R^{a}, wherein R^{a} is 3-8 membered heterocyclyl, wherein the 3-8 membered heterocyclyl of R^{a} is optionally substituted with one or more R^{c}. In some embodiments, R² is methyl, wherein the methyl of R² is substituted with one or more R^{a}, wherein R^{a} is 3-8 membered heterocyclyl, wherein the 3-8 membered heterocyclyl of R^{a} is optionally substituted with one or more oxo or C₁₋₆alkyl. In some embodiments, R² is

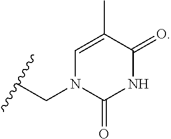 [SMILES: *Cn1cc(C)c(=O)[nH]c1=O]

In some embodiments of a compound of formula (I-B1), or a stereoisomer or tautomer thereof, or a pharmaceutically acceptable salt of any of the foregoing, R² is C₁₋₆alkyl, wherein the C₁₋₆alkyl of R² is substituted with one or more R^{a}, wherein R^{a} is —O—R^{e}. In some embodiments, R² is methyl, wherein the methyl of R² is substituted with one or more R^{a}, wherein R^{a} is —O—R^{e}. In some embodiments, R² is methyl, wherein the methyl of R² is substituted with one or more R^{a}, wherein R^{a} is —O—R^{e}, wherein R^{e} is —C(O)-(3-15 membered heterocyclyl). In some embodiments, R² is

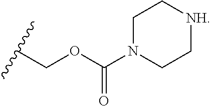 [SMILES: *COC(=O)N1CCNCC1]

In some embodiments of a compound of formula (I-B1), or a stereoisomer or tautomer thereof, or a pharmaceutically acceptable salt of any of the foregoing, R² is C₁₋₆alkyl, wherein the C₁₋₆alkyl of R² is substituted with one or more R^{a}, wherein R^{a} is —N(R^{c})(R^{d}). In some embodiments, R² is methyl, wherein the methyl of R² is substituted with one or more R^{a}, wherein R^{a} is —N(R^{c})(R^{d}). In some embodiments, R² is methyl, wherein the methyl of R² is substituted with one or more R^{a}, wherein R^{a} is —N(R^{c})(R^{d}), wherein one of R^{c} and R^{d} is H, and the other of R^{c} and R^{d} is —C(O)—N(C₁₋₆alkyl)₂. In some embodiments, R² is

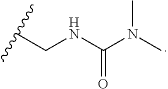 [SMILES: *CNC(=O)N(C)C]

In some embodiments, provided herein is a compound of formula (I-1) or formula (I-B), or a stereoisomer or tautomer thereof, or a pharmaceutically acceptable salt of any of the foregoing, wherein the compound is a compound of formula (I-B2):

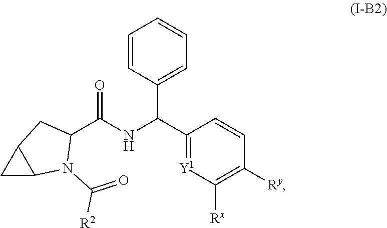

or a stereoisomer or tautomer thereof, or a pharmaceutically acceptable salt of any of the foregoing, or any variation or embodiment thereof, wherein Y¹ is CR^{x} or N; wherein, when the ring bearing R^{x}, and R^{y} is phenyl, R^{x}, and R^{y} are each independently H, halo, C₁₋₆alkyl, C₂₋₆alkenyl, —NH₂, —NH—C(O)—(C₁₋₆alkyl), —NH—C(O)-(3-15 membered heterocyclyl), C₃₋₁₀cycloalkyl, or 5-20 membered heteroaryl, wherein the C₁₋₆alkyl is optionally substituted with one or more halo, —NH—C(O)—NH(C₁₋₆alkyl), —NH—C(O)—C₁₋₆alkyl, or —NH—C(O)—C₁₋₆alkoxy, the C₃₋₁₀cycloalkyl is optionally substituted with one or more halo or C₁₋₆alkyl, and the 5-20 membered heteroaryl is optionally substituted with one or more C₁₋₆alkyl; and wherein when the ring bearing R^{x}, and R^{y} is pyridyl, R^{x}, and R^{y} are each independently H, halo, C₁₋₆alkyl, C₂₋₆alkenyl, C₁₋₆alkoxy, —NH₂, or C₃₋₁₀cycloalkyl, wherein, the C₁₋₆alkyl is optionally substituted with one or more halo, and the C₃₋₁₀cycloalkyl is optionally substituted with one or more halo or C₁₋₆alkyl. In some embodiments, Y¹ is CH or N; R^{x} is H or fluoro; and R^{y} is (i) isopropyl or (ii) C₃₋₄cycloalkyl. In some embodiments, Y¹ is CH; R^{x} is H or fluoro; and R^{y} is (i) isopropyl or (ii) C₃₋₄cycloalkyl. In some embodiments, Y¹ is N; R^{x} is H or fluoro; and R^{y} is (i) isopropyl or (ii) C₃₋₄cycloalkyl.

In some embodiments of a compound of formula (I-B2), or a stereoisomer or tautomer thereof, or a pharmaceutically acceptable salt of any of the foregoing, R² is C₁₋₆alkyl, wherein the C₁₋₆alkyl of R² is optionally substituted with one or more R^{a}. In some embodiments, R² is C₁₋₆alkyl, wherein the C₁₋₆alkyl of R² is substituted with one or more R^{a}, wherein R^{a} is —OH or 5-20 membered heteroaryl, wherein the 5-20 membered heteroaryl of R^{a} is optionally substituted with one or more R^{b}. In some embodiments, R² is methyl, wherein the methyl of R² is substituted with one or more R^{a}, wherein R^{a} is —OH or 5-10 membered heteroaryl, wherein the 5-10 membered heteroaryl of R^{a} is optionally substituted with one or more R^{b}. In some embodiments, R² is methyl, wherein the methyl of R² is substituted with one or more R^{a}, wherein R^{a} is —OH or 5-10 membered heteroaryl, wherein the 5-10 membered heteroaryl of R^{a} is optionally substituted with one or more C₁₋₆alkyl, wherein the C₁₋₆alkyl is optionally substituted with one or more halo, —NH₂, —NH(C₁₋₆alkyl), —N(C₁₋₆alkyl)₂, —NH—C(O)C₁₋₆alkyl, or —NH—C(O)—C₁₋₆alkoxy. In some embodiments, R² is methyl, wherein the methyl of R² is substituted with one or more R^{a}, wherein R^{a} is —OH or 5-10 membered heteroaryl, wherein the 5-10 membered heteroaryl of R^{a} is optionally substituted with one or more methyl, wherein the methyl is optionally substituted with one or more fluoro.

In some embodiments of a compound of formula (I-B2), or a stereoisomer or tautomer thereof, or a pharmaceutically acceptable salt of any of the foregoing, R² is selected from the group consisting of

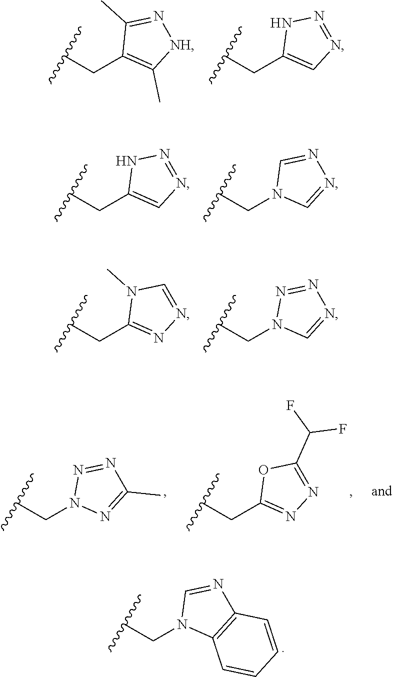

In some embodiments, R² is

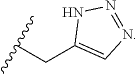 [SMILES: *Cc1cnn[nH]1]

In some embodiments of a compound of formula (I-B2), or a stereoisomer or tautomer thereof, or a pharmaceutically acceptable salt of any of the foregoing, R² is C₁₋₆alkyl, wherein the C₁₋₆alkyl of R² is substituted with one or more R^{a}, wherein R^{a} is 3-15 membered heterocyclyl, wherein the 3-15 membered heterocyclyl of R^{a} is optionally substituted with one or more R^{c}. In some embodiments, R² is methyl, wherein the methyl of R² is substituted with one or more R^{a}, wherein R^{a} is 3-15 membered heterocyclyl, wherein the 3-15 membered heterocyclyl of R^{a} is optionally substituted with one or more R^{c}. In some embodiments, R² is methyl, wherein the methyl of R² is substituted with one or more R^{a}, wherein R^{a} is 3-8 membered heterocyclyl, wherein the 3-8 membered heterocyclyl of R^{a} is optionally substituted with one or more R^{c}. In some embodiments, R² is methyl, wherein the methyl of R² is substituted with one or more R^{a}, wherein R^{a} is 3-8 membered heterocyclyl, wherein the 3-8 membered heterocyclyl of R^{a} is optionally substituted with one or more oxo or C₁₋₆alkyl. In some embodiments, R² is

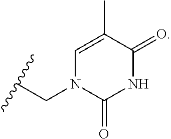 [SMILES: *Cn1cc(C)c(=O)[nH]c1=O]

In some embodiments of a compound of formula (I-B2), or a stereoisomer or tautomer thereof, or a pharmaceutically acceptable salt of any of the foregoing, R² is C₁₋₆alkyl, wherein the C₁₋₆alkyl of R² is substituted with one or more R^{a}, wherein R^{a} is —O—R^{e}. In some embodiments, R² is methyl, wherein the methyl of R² is substituted with one or more R^{a}, wherein R^{a} is —O—R^{e}. In some embodiments, R² is methyl, wherein the methyl of R² is substituted with one or more R^{a}, wherein R^{a} is —O—R^{e}, wherein R^{e} is —C(O)-(3-15 membered heterocyclyl). In some embodiments, R² is

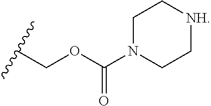 [SMILES: *COC(=O)N1CCNCC1]

In some embodiments of a compound of formula (I-B2), or a stereoisomer or tautomer thereof, or a pharmaceutically acceptable salt of any of the foregoing, R² is C₁₋₆alkyl, wherein the C₁₋₆alkyl of R² is substituted with one or more R^{a}, wherein R^{a} is —N(R^{c})(R^{d}). In some embodiments, R² is methyl, wherein the methyl of R² is substituted with one or more R^{a}, wherein R^{a} is —N(R^{c})(R^{d}). In some embodiments, R² is methyl, wherein the methyl of R² is substituted with one or more R^{a}, wherein R^{a} is —N(R^{c})(R^{d}), wherein one of R^{c} and R^{d} is H, and the other of R^{c} and R^{d} is —C(O)—N(C₁₋₆alkyl)₂. In some embodiments, R² is

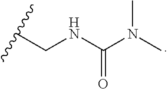 [SMILES: *CNC(=O)N(C)C]

In some embodiments, provided herein is a compound of formula (I-1), or a stereoisomer or tautomer thereof, or a pharmaceutically acceptable salt of any of the foregoing, wherein the compound is a compound of formula (I-C):

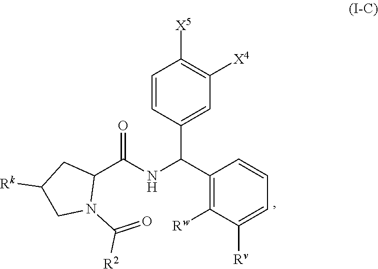

or a stereoisomer or tautomer thereof, or a pharmaceutically acceptable salt of any of the foregoing, or any variation or embodiment thereof, wherein X⁵ is H, C₁₋₆alkyl, C₁₋₆alkoxy, or C₃₋₁₀cycloalkyl; X⁴ is H, halo, C₁₋₆alkyl, C₁₋₆alkoxy, or 5-20 membered heteroaryl; R^{v} is —NH₂, —NH—C(O)—(C₁₋₆alkyl), or —NH—C(O)-(3-15 membered heterocyclyl); and R^{w} is H, —NH₂, —NH—C(O)—(C₁₋₆alkyl), or —NH—C(O)-(3-15 membered heterocyclyl). In some embodiments, X⁵ is H or C₁₋₆alkyl; X⁴ is H; R^{v} is —NH₂, —NH—C(O)—(C₁₋₆alkyl), or —NH—C(O)-(3-15 membered heterocyclyl); and R^{w} is H, —NH₂, —NH—C(O)—(C₁₋₆alkyl), or —NH—C(O)-(3-15 membered heterocyclyl). In some embodiments, R^{w} is H and R^{v} is —NH—C(O)C₁₋₆alkyl. In some embodiments, R^{w} is H and R^{v} is —NH—C(O)CH₃.

In some embodiments, provided is a compound of formula (I-1), or a stereoisomer or tautomer thereof, or a pharmaceutically acceptable salt of any of the foregoing, wherein the compound is a compound of formula (I-D):

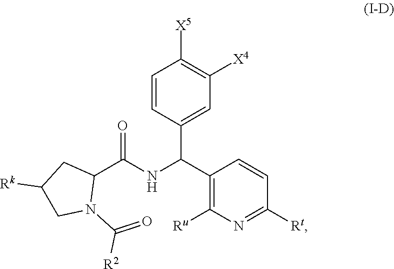

or a stereoisomer or tautomer thereof, or a pharmaceutically acceptable salt of any of the foregoing, or any variation or embodiment thereof, wherein X⁵ is H, C₁₋₆alkyl, C₁₋₆alkoxy, or C₃₋₁₀cycloalkyl; X⁴ is H, halo, C₁₋₆alkyl, C₁₋₆alkoxy, or 5-20 membered heteroaryl; and R^{t} and R^{u} are independently H, C₁₋₆alkoxy, or —NH₂. In some embodiments, X⁵ is C₁₋₆alkyl; X⁴ is H, halo, or C₁₋₆alkyl; and R^{t} and R^{u} are independently H or —NH₂. In some embodiments, at least one of R^{t} and R^{u} is —NH₂. In some embodiments, R^{t} is H and R^{u} is —NH₂. In some embodiments, R^{t} is —NH₂ and R^{u} is H.

In some embodiments of a compound of formula (I-C) or (I-D), or a stereoisomer or tautomer thereof, or a pharmaceutically acceptable salt of any of the foregoing, R^{k} is H or halo.

In some embodiments of a compound of formula (I-C) or (I-D), or a stereoisomer or tautomer thereof, or a pharmaceutically acceptable salt of any of the foregoing, R^{k} is H or fluoro. In some embodiments of a compound of formula (I-C) or (I-D), or a stereoisomer or tautomer thereof, or a pharmaceutically acceptable salt of any of the foregoing, R^{k} is fluoro.

In some embodiments, provided is a compound of formula (I-1), wherein the compound is a compound of formula (I-D1):

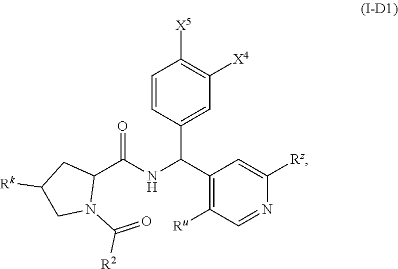

or a stereoisomer or tautomer thereof, or a pharmaceutically acceptable salt of any of the foregoing, or any variation or embodiment thereof, wherein X⁵ is H, C₁₋₆alkyl, C₁₋₆alkoxy, or C₃₋₁₀cycloalkyl; X⁴ is H, halo, C₁₋₆alkyl, C₁₋₆alkoxy, or 5-20 membered heteroaryl; and R^{t} and R^{u} are independently H, C₁₋₆alkoxy, or —NH₂. In some embodiments, X⁵ is C₁₋₆alkyl; X⁴ is H, halo, or C₁₋₆alkyl; and R^{u} and R^{z} are independently H, halo or —NH₂. In some embodiments, at least one of R^{u} and R^{z} is —NH₂. In some embodiments, R^{u} is H and R^{z} is —NH₂. In some embodiments, R^{u} is —NH₂ and R^{z} is H. In some embodiments, at least one of R^{u} and R^{z} is halo. In some embodiments, R^{u} is H and R^{z} is fluoro. In some embodiments, R^{u} is fluoro and R^{z} is H.

In some embodiments, provided is a compound of formula (I-1), wherein the compound is a compound of formula (I-D2):

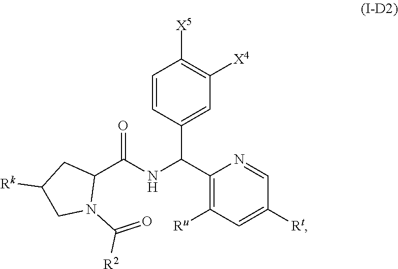

or a stereoisomer or tautomer thereof, or a pharmaceutically acceptable salt of any of the foregoing, or any variation or embodiment thereof, wherein X⁵ is H, C₁₋₆alkyl, C₁₋₆alkoxy, or C₃₋₁₀cycloalkyl; X⁴ is H, halo, C₁₋₆alkyl, C₁₋₆alkoxy, or 5-20 membered heteroaryl, wherein the C₁₋₆alkyl of X⁴ is optionally substituted with one of more halo; and R^{t} and R^{u} are independently H, C₁₋₆alkoxy, or —NH₂. In some embodiments, X⁵ is C₁₋₆alkyl; X⁴ is H, halo, or C₁₋₆alkyl; and R^{u} and R^{z} are independently H, halo or —NH₂. In some embodiments, at least one of R^{u} and R^{z} is —NH₂. In some embodiments, R^{u} is H and R^{z} is —NH₂. In some embodiments, R^{u} is —NH₂ and R^{z} is H. In some embodiments, at least one of R^{u} and R^{z} is halo. In some embodiments, R^{u} is H and R^{z} is fluoro. In some embodiments, R^{u} is fluoro and R^{z} is H.

In some embodiments, provided is a compound of formula (I-1), wherein the compound is a compound of formula (I-E):

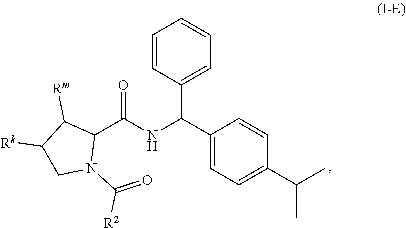

or a stereoisomer or tautomer thereof, or a pharmaceutically acceptable salt of any of the foregoing, or any variation or embodiment thereof, wherein R^{k} and R^{m} are independently H, OH, —NH₂, or —NH—C(O)C₁₋₆alkyl. In some embodiments, R^{k} is H and R^{m} is H, OH, —NH₂, or —NH—C(O)C₁₋₆alkyl. In some embodiments, R^{k} is H and R^{m} is OH. In some embodiments, R^{k} is H, OH, —NH₂, or —NH—C(O)C₁₋₆alkyl, and R^{m} is H. In some embodiments, R^{k} is OH, —NH₂, or —NH—C(O)C₁₋₆alkyl, and R^{m} is H. In some embodiments, R^{k} is OH, —NH₂, or —NH—C(O)CH₃, and R^{m} is H.

In some embodiments, provided is a compound of formula (I-1), or a stereoisomer or tautomer thereof, or a pharmaceutically acceptable salt of any of the foregoing, wherein the compound is a compound of formula (I-F):

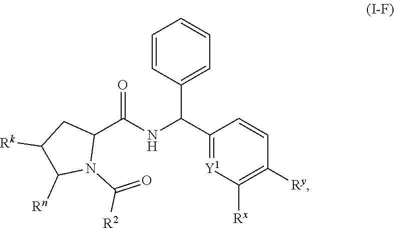

or a stereoisomer or tautomer thereof, or a pharmaceutically acceptable salt of any of the foregoing, or any variation or embodiment thereof, wherein Y¹ is CH or N; R^{x} is H, halo, C₁₋₆alkyl, or —NH₂, wherein, when Y¹ is CH, the C₁₋₆alkyl of R^{x} may be optionally substituted with one or more halo; R^{y} is (i) C₁₋₆alkyl, (ii), C₂₋₆alkenyl or (iii) C₃₋₁₀cycloalkyl, wherein the C₃₋₁₀cycloalkyl is optionally substituted with one or more halo or C₁₋₆alkyl; R^{k} is H, halo, —OH, —NH₂, or —NH—C(O)C₁₋₆alkyl; and Rⁿ is H, C₁₋₆alkyl, or C₃₋₁₀cycloalkyl. In some embodiments, Y¹ is CH or N; R^{x} is H, halo, or C₁₋₆alkyl; R^{y} is (i) C₁₋₆alkyl, or (ii) C₃₋₁₀cycloalkyl, wherein the C₃₋₁₀cycloalkyl is optionally substituted with one or more halo or C₁₋₆alkyl; R^{k} is H, halo, —OH, —NH₂, or —NH—C(O)C₁₋₆alkyl; and Rⁿ is H, C₁₋₆alkyl, or C₃₋₁₀cycloalkyl.

In some embodiments of a compound of formula (I-F), Y¹ is CH or N; R^{x} is H, halo, or C₁₋₆alkyl; R^{y} is (i) C₁₋₆alkyl, (ii), C₂₋₆alkenyl, or (iii) C₃₋₁₀cycloalkyl, wherein the C₃₋₁₀cycloalkyl is optionally substituted with one or more halo or C₁₋₆alkyl; R^{k} is H or halo; and Rⁿ is H, C₁₋₆alkyl, or C₃₋₆cycloalkyl. In some embodiments, Y¹ is N or CH, R^{x} is H or halo, R^{y} is C₁₋₆alkyl or C₃₋₆cycloalkyl, R^{k} is H or halo, and Rⁿ is C₁₋₆alkyl or C₃₋₆cycloalkyl. In some embodiments, Y¹ is N or CH, R^{x} is H or fluoro, R^{y} is C₁₋₆alkyl or C₃₋₆cycloalkyl, R^{k} is H or fluoro, and Rⁿ is C₁₋₆alkyl or C₃₋₆cycloalkyl. In some embodiments, Y¹ is N or CH, R^{x} is H or fluoro, R^{y} is C₁₋₆alkyl or C₃₋₆cycloalkyl, R^{k} is H or fluoro, and R^{y} is C₁₋₆alkyl or C₃₋₆cycloalkyl. In some embodiments, Y¹ is N or CH, R^{x} is H or fluoro, R^{y} is C₁₋₆alkyl or C₃₋₆cycloalkyl, R^{k} is H, and Rⁿ is C₁₋₆alkyl or C₃₋₆cycloalkyl. In some embodiments, Y¹ is N or CH, R^{x} is H or fluoro, R^{y} is C₁₋₃alkyl or C₃₋₆cycloalkyl, R^{k} is H, and Rⁿ is C₁₋₃alkyl or C₃₋₆cycloalkyl.

In some embodiments of a compound of formula (I-C), (I-D), (I-E), or (I-F), or a stereoisomer or tautomer thereof, or a pharmaceutically acceptable salt of any of the foregoing, R² is C₁₋₆alkyl, wherein the C₁₋₆alkyl of R² is optionally substituted with one or more R^{a}. In some embodiments, R² is C₁₋₆alkyl, wherein the C₁₋₆alkyl of R² is substituted with one or more R^{a}, wherein R^{a} is —OH or 5-20 membered heteroaryl, wherein the 5-20 membered heteroaryl of R^{a} is optionally substituted with one or more R^{b}. In some embodiments, R² is methyl, wherein the methyl of R² is substituted with one or more R^{a}, wherein R^{a} is —OH or 5-10 membered heteroaryl, wherein the 5-10 membered heteroaryl of R^{a} is optionally substituted with one or more R^{b}. In some embodiments, R² is methyl, wherein the methyl of R² is substituted with one or more R^{a}, wherein R^{a} is —OH or 5-10 membered heteroaryl, wherein the 5-10 membered heteroaryl of R^{a} is optionally substituted with one or more C₁₋₆alkyl, wherein the C₁₋₆alkyl is optionally substituted with one or more halo, —NH₂, —NH(C₁₋₆alkyl), —N(C₁₋₆alkyl)₂, —NH—C(O)C₁₋₆alkyl, or —NH—C(O)—C₁₋₆alkoxy. In some embodiments, R² is methyl, wherein the methyl of R² is substituted with one or more R^{a}, wherein R^{a} is —OH or 5-10 membered heteroaryl, wherein the 5-10 membered heteroaryl of R^{a} is optionally substituted with one or more methyl, wherein the methyl is optionally substituted with one or more fluoro.

In some embodiments of a compound of formula (I-C), (I-D), (I-E), or (I-F), or a stereoisomer or tautomer thereof, or a pharmaceutically acceptable salt of any of the foregoing, R² is selected from the group consisting of

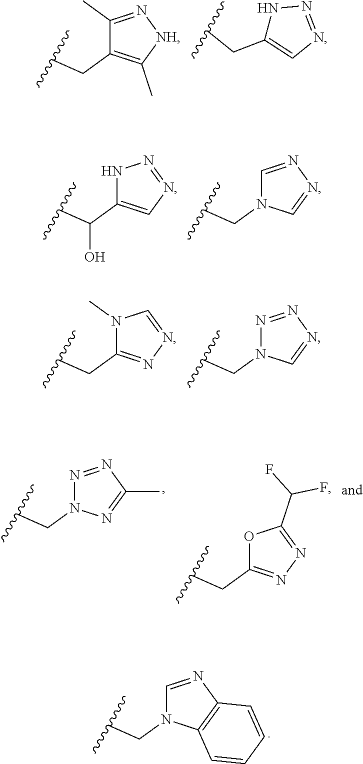

In some embodiments, R² is.

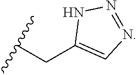 [SMILES: *Cc1cnn[nH]1]

In some embodiments of a compound of formula (I-C), (I-D), (I-E), or (I-F), or a stereoisomer or tautomer thereof, or a pharmaceutically acceptable salt of any of the foregoing, R² is selected from the group consisting of

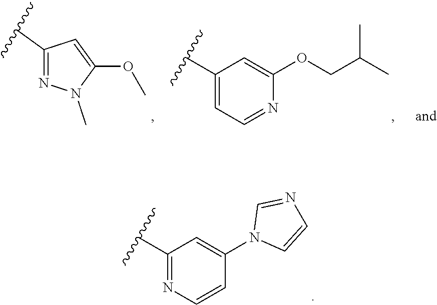

In some embodiments of a compound of formula (I-C), (I-D), (I-E), or (I-F), or a stereoisomer or tautomer thereof, or a pharmaceutically acceptable salt of any of the foregoing, R² is selected from the group consisting of

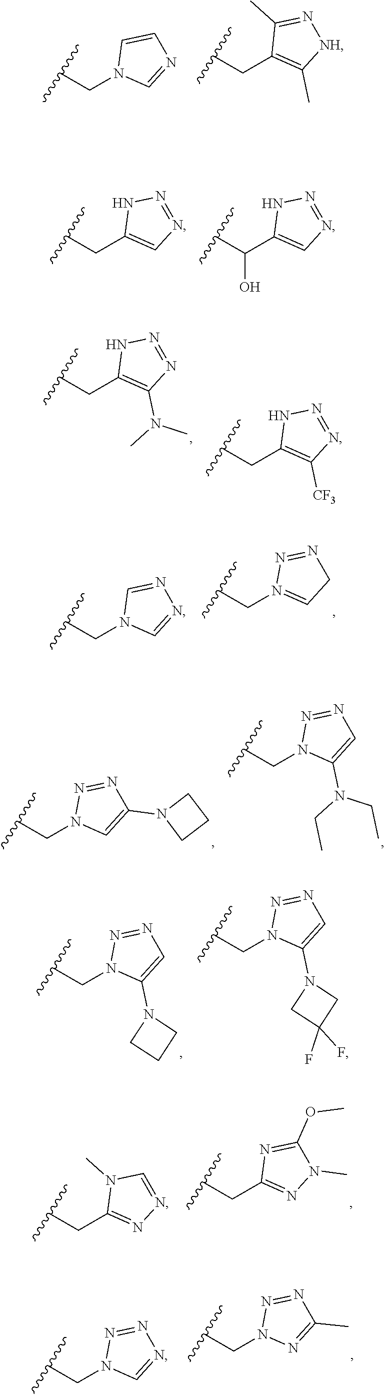

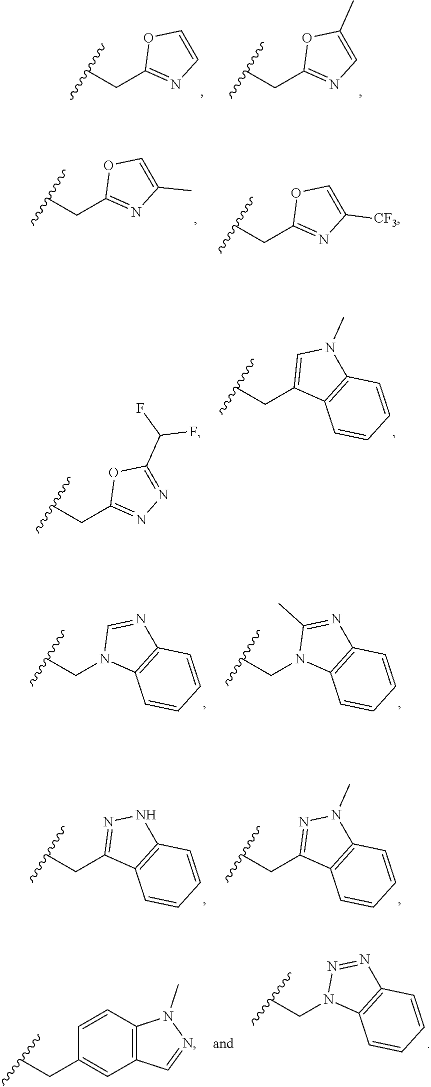

In some embodiments, R² is

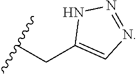 [SMILES: *Cc1cnn[nH]1]

In some embodiments of a compound of formula (I-C), (I-D), (I-E), or (I-F), or a stereoisomer or tautomer thereof, or a pharmaceutically acceptable salt of any of the foregoing, R² is

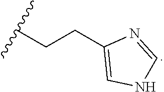 [SMILES: *CCc1c[nH]cn1]

In some embodiments of a compound of formula (I-C), (I-D), (I-E), or (I-F), or a stereoisomer or tautomer thereof, or a pharmaceutically acceptable salt of any of the foregoing, R² is methyl, wherein the methyl of R² is substituted with one or more R^{a}, wherein R^{a} is 3-8 membered heterocyclyl, wherein the 3-8 membered heterocyclyl of R^{a} is optionally substituted with one or more R^{c}, wherein R^{c} is oxo, C₁₋₆alkyl, or —C(O)—C₁₋₆alkoxy, wherein the C₁₋₆alkyl of R^{c} is optionally substituted with one or more halo, and the —C(O)—C₁₋₆alkoxy of R^{c} is optionally substituted with one or more halo. In some embodiments, R² is selected from the group consisting of

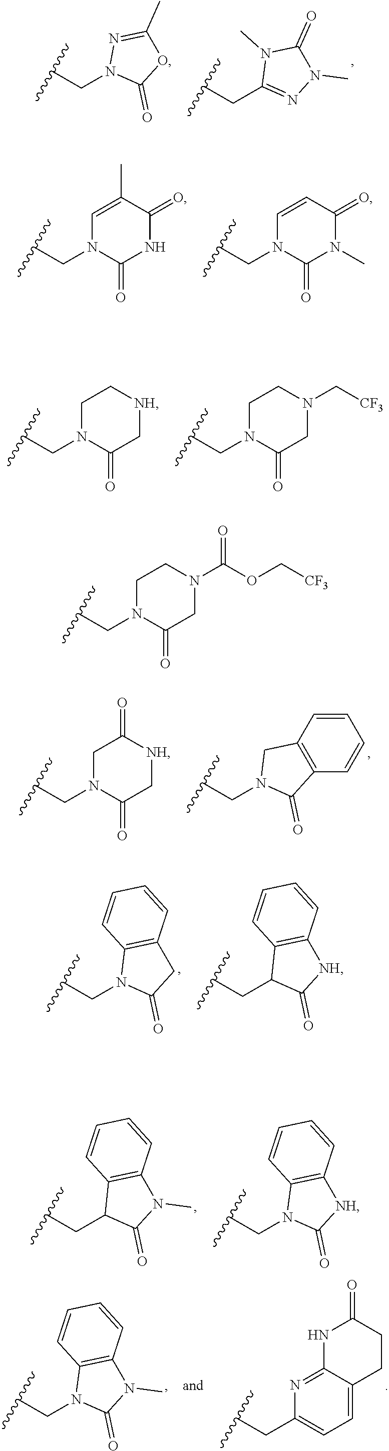

In some embodiments of a compound of formula (I-C), (I-D), (I-E), or (I-F), or a stereoisomer or tautomer thereof, or a pharmaceutically acceptable salt of any of the foregoing, R² is methyl, wherein the methyl of R² is substituted with one or more R^{a}, wherein R^{a} is —O—R^{e}, wherein R^{e} is —C(O)-(3-15 membered heterocyclyl) wherein the —C(O)-(3-15 membered heterocyclyl) of R^{e} is optionally substituted with one or more C₁₋₆alkyl, wherein the C₁₋₆alkyl is optionally substituted with one or more halo, C₁₋₆alkoxy, or C₃₋₁₀cycloalkyl. In some embodiments, R² is selected from the group consisting of

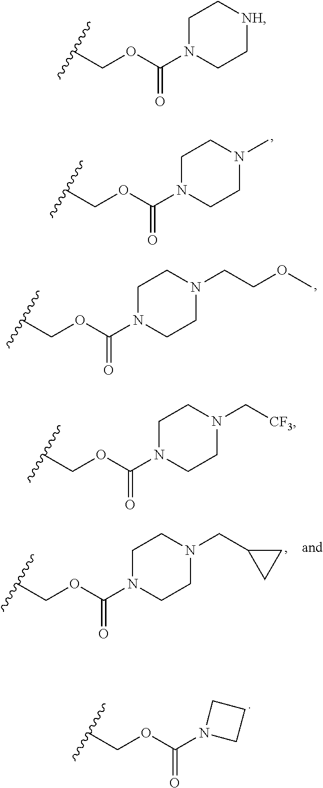

In some embodiments of a compound of formula (I-C), (I-D), (I-E), or (I-F), or a stereoisomer or tautomer thereof, or a pharmaceutically acceptable salt of any of the foregoing, R² is methyl, wherein the methyl of R² is substituted with one or more R^{a}, wherein R^{a} is —N(R^{c})(R^{d}), wherein one of R^{c} and R^{d} is H, and the other of R^{c} and R^{d}, —C(O)—C₁₋₆alkyl, —C(O)—N(C₁₋₆alkyl)₂, or —C(O)-(3-15 membered heterocyclyl). In some embodiments, R² is

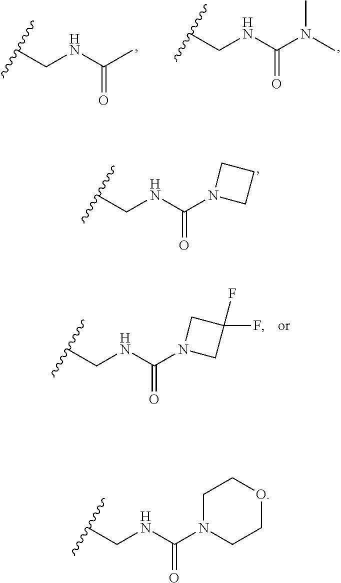

In some embodiments, provided herein is a compound of formula (I-1), wherein the compound is a compound of formula (I-G):

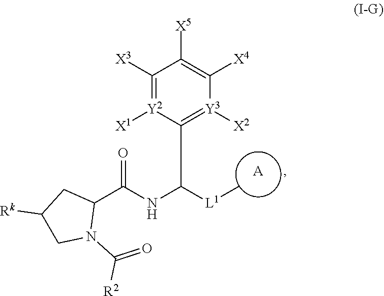

or a stereoisomer or tautomer thereof, or a pharmaceutically acceptable salt of any of the foregoing, wherein ring A is (i) 3-15 membered heterocyclyl, wherein the 3-15 membered heterocyclyl of ring A is optionally substituted with one or more oxo, (ii) 5-20 membered heteroaryl, wherein the 5-20 membered heteroaryl of ring A is optionally substituted with one or more halo, C₁₋₆alkyl, C₂₋₆alkenyl, C₁₋₆alkoxy, —NH₂, or C₃₋₁₀cycloalkyl, wherein, the C₁₋₆alkyl is optionally substituted with one or more halo, and the C₃₋₁₀cycloalkyl is optionally substituted with one or more halo or C₁₋₆alkyl, or (iii) C₃₋₁₀cycloalkyl.

In some embodiments of a compound of formula (I-G), or a stereoisomer or tautomer thereof, or a pharmaceutically acceptable salt of any of the foregoing, X³ is H, fluoro or methyl optionally substituted with one or more fluoro; X⁴ is (i) isopropyl (ii) C₃₋₄cycloalkyl optionally substituted with one or more halo or C₁₋₆alkyl or (iii) butyl; and R^{z} is fluoro or methyl; provided that at least one of X³ and X⁴ is halo, CF₂ or CF₃.

In some embodiments of a compound of formula (I-G), or a stereoisomer or tautomer thereof, or a pharmaceutically acceptable salt of any of the foregoing, R^{k} is H or halo. In some embodiments of a compound of formula (I-G), or a stereoisomer or tautomer thereof, or a pharmaceutically acceptable salt of any of the foregoing, R^{k} is H or fluoro. In some embodiments of a compound of formula (I-G), or a stereoisomer or tautomer thereof, or a pharmaceutically acceptable salt of any of the foregoing, R^{k} is fluoro.

In some embodiments of a compound of formula (I-G), or a stereoisomer or tautomer thereof, or a pharmaceutically acceptable salt of any of the foregoing, R² is 5-20 membered heteroaryl or —(C₁₋₄alkyl)(5-20 membered heteroaryl), wherein the C₁₋₄ alkyl is optionally substituted with one or more or more —OH, halo, —NH₂, —NH(C₁₋₆alkyl), —N(C₁₋₆alkyl)₂, and wherein the 5-20 membered heteroaryl is optionally substituted with one or more R^{s}. In some embodiments, R^{s} is halo, C₁₋₆alkyl, C₁₋₆alkoxy, —NH₂, —NH(C₁₋₆alkyl), —N(C₁₋₆alkyl)₂, —NH—C(O)—C₁₋₆alkyl, C₆₋₂₀aryl, C₃₋₁₀cycloalkyl, 3-15 membered heterocyclyl, 5-20 membered heteroaryl, or —C(O)—C₁₋₆alkoxy. In some embodiments, the C₁₋₆alkyl of R^{s} is optionally substituted with one or more halo, C₁₋₆alkoxy, —NH₂, —NH(C₁₋₆alkyl), —N(C₁₋₆alkyl)₂, —NH—C(O)C₁₋₆alkyl, or —NH—C(O)—C₁₋₆alkoxy, and the 3-15-membered heterocyclyl of R^{s} is optionally substituted with one or more halo or —C(O)—C₁₋₆alkoxy.

In some embodiments of a compound of formula (I-G), or a stereoisomer or tautomer thereof, or a pharmaceutically acceptable salt of any of the foregoing, R² is selected from the group consisting of

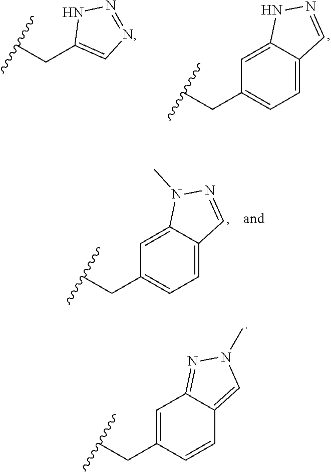 [SMILES: *Cc1cnn[nH]1 ; *Cc1ccc2cn[nH]c2c1 ; *Cc1ccc2cnn(C)c2c1 ; *Cc1ccc2cn(C)nc2c1]

In some embodiments of a compound of formula (I-G), or a stereoisomer or tautomer thereof, or a pharmaceutically acceptable salt of any of the foregoing, R² is methyl, wherein the methyl of R² is substituted with one or more R^{a}, wherein R^{a} is —N(R^{c})(R^{d}), wherein one of R^{c} and R^{d} is H, and the other of R^{c} and R^{d}, —C(O)—C₁₋₆alkyl, —C(O)—N(C₁₋₆alkyl)₂, or —C(O)-(3-15 membered heterocyclyl). In some embodiments, R² is

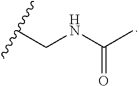 [SMILES: *CNC(C)=O]

In some embodiments of a compound of formula (I-G), or a stereoisomer or tautomer thereof, or a pharmaceutically acceptable salt of any of the foregoing, ring A is (i) 3-15 membered heterocyclyl, wherein the 3-15 membered heterocyclyl of ring A is optionally substituted with one or more oxo, or C₁₋₆alkyl, (ii) 5-20 membered heteroaryl, wherein the 5-20 membered heteroaryl of ring A is optionally substituted with one or more halo, C₁₋₆alkyl, C₂₋₆alkenyl, C₁₋₆alkoxy, —NH₂, or C₃₋₁₀cycloalkyl, wherein, the C₁₋₆alkyl is optionally substituted with one or more halo, and the C₃₋₁₀cycloalkyl is optionally substituted with one or more halo or C₁₋₆alkyl, or (iii) C₃₋₁₀cycloalkyl.

In some embodiments of a compound of formula (I-G), or a stereoisomer or tautomer thereof, or a pharmaceutically acceptable salt of any of the foregoing, ring A is 3-15 membered heterocyclyl, wherein the 3-15 membered heterocyclyl of ring A is optionally substituted with one or more oxo, or C₁₋₆alkyl. In some embodiments ring A is selected from the group consisting of

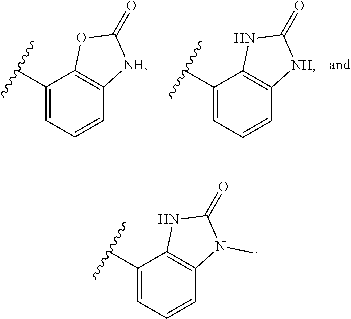 [SMILES: *c1cccc2[nH]c(=O)oc12 ; *c1cccc2[nH]c(=O)[nH]c12 ; *c1cccc2c1[nH]c(=O)n2C]

In some embodiments of a compound of formula (I-G), or a stereoisomer or tautomer thereof, or a pharmaceutically acceptable salt of any of the foregoing, ring A is 5-20 membered heteroaryl, wherein the 5-20 membered heteroaryl of ring A is optionally substituted with one or more halo, C₁₋₆alkyl, C₂₋₆alkenyl, C₁₋₆alkoxy, —NH₂, or C₃₋₁₀cycloalkyl, wherein, the C₁₋₆alkyl is optionally substituted with one or more halo, and the C₃₋₁₀cycloalkyl is optionally substituted with one or more halo or C₁₋₆alkyl. In some embodiments ring A is 5-20 membered heteroaryl, wherein the 5-20 membered heteroaryl of ring A is optionally substituted with one or more C₁₋₆alkyl. In some embodiments ring A is selected from the group consisting of

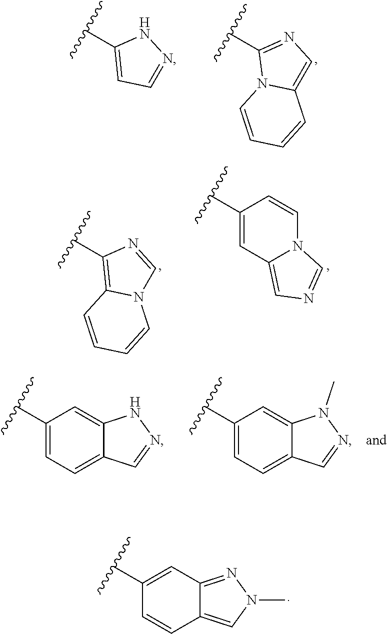

In some embodiments of a compound of formula (I-G), or a stereoisomer or tautomer thereof, or a pharmaceutically acceptable salt of any of the foregoing, ring A is C₃₋₁₀cycloalkyl. In some embodiments, ring A is C₃₋₆cycloalkyl. In some embodiments ring A is cyclopropyl.

In some embodiments, provided herein is a compound of formula (I-1), wherein the compound is a compound of formula (I-H):

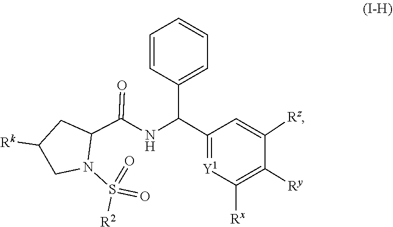

or a stereoisomer or tautomer thereof, or a pharmaceutically acceptable salt of any of the foregoing, wherein Y¹ is CR^{x} or N; wherein, when the ring bearing R^{x}, R^{y} and R^{z} is phenyl, R^{x}, R^{y} and R^{z} is H, halo, C₁₋₆alkyl, C₂₋₆alkenyl, —NH₂, —NH—C(O)—(C₁₋₆alkyl), —NH—C(O)-(3-15 membered heterocyclyl), C₃₋₁₀cycloalkyl, or 5-20 membered heteroaryl, wherein the C₁₋₆alkyl is optionally substituted with one or more halo, —NH—C(O)—NH(C₁₋₆alkyl), —NH—C(O)—C₁₋₆alkyl, or —NH—C(O)—C₁₋₆alkoxy, the C₃₋₁₀cycloalkyl is optionally substituted with one or more halo or C₁₋₆alkyl, and the 5-20 membered heteroaryl is optionally substituted with one or more C₁₋₆alkyl; and wherein when the ring bearing R^{x}, R^{y} and R^{z} is pyridyl, R^{x}, R^{y} and R^{z} are each interpedently H, halo, C₁₋₆alkyl, C₂₋₆alkenyl, C₁₋₆alkoxy, —NH₂, or C₃₋₁₀cycloalkyl, wherein, the C₁₋₆alkyl is optionally substituted with one or more halo, and the C₃₋₁₀cycloalkyl is optionally substituted with one or more halo or C₁₋₆alkyl.

In some embodiments, provided herein is a compound of formula (I-1), such as a compound of formula (I), (I-A), (I-A1), (I-A2), (I-A3), (I-A4), (I-B), (I-B1), (I-B2), (I-C), (I-D), (I-D1), (I-D2), (I-E), (I-F), (I-G), or (I-H) or a stereoisomer or tautomer thereof, or a pharmaceutically acceptable salt of any of the foregoing, wherein R² is C₁₋₆alkyl, wherein the C₁₋₆alkyl of R² is optionally substituted with one or more R^{a}. In some embodiments, R² is C₃₋₁₀cycloalkyl, wherein the C₃₋₁₀cycloalkyl of R² is optionally substituted with one or more R^{q}. In other embodiments, R² is 3-15 membered heterocyclyl, wherein the 3-15 membered heterocyclyl of R² is optionally substituted with one or more halo, oxo, C₁₋₆alkyl, —C(O)—C₁₋₆alkyl, or 5-20 membered heteroaryl. In some embodiments, R² is 5-20 membered heteroaryl, or —(C₁₋₄alkyl)(5-20 membered heteroaryl), wherein the C₁₋₄ alkyl is optionally substituted with one or more or more —OH, halo, —NH₂, —NH(C₁₋₆alkyl), —N(C₁₋₆alkyl)₂, and wherein the 5-20 membered heteroaryl is optionally substituted with one or more R^{s}. In some embodiments, R² is —N(R^{g})(R^{h}), wherein R^{g} and R^{h} are independently H or C₁₋₆alkyl. In some embodiments, R² is —C(O)—R^{j}, wherein R^{j} is C₃₋₁₀cycloalkyl, —NH(C₁₋₆alkyl), —N(C₁₋₆alkyl)₂, or —NH(5-20 membered heteroaryl). In some embodiments, R² is C₆₋₂₀aryl, wherein the C₆₋₂₀aryl of R² is optionally substituted with one or more 5-20 membered heteroaryl or —O(R^{p}), wherein R^{p} is 3-15 membered heterocyclyl, wherein the 3-15 membered heterocyclyl of R^{p} is optionally substituted with one or more —C(O)—C₁₋₆alkyl.

In some embodiments of a compound of formula (I), or any variation or embodiment thereof, or a stereoisomer or tautomer thereof, or a pharmaceutically acceptable salt of any of the foregoing, (G1-Z1) and (G2-Z2) are each independently a moiety selected from Table 1A. In some embodiments, the (G1-Z1) and (G2-Z2) moieties selected from Table 1A are each independently substituted with a group bound to L. In some embodiments, the (G1-Z1) and (G2-Z2) moieties selected from Table 1A are each independently bound to L. In some embodiments, L is bound at any available position. In some embodiments, L is bound at the group corresponding to R². In some embodiments, L is bound at the group corresponding to Rⁿ.

Compound Names included in Table 1A and for all intermediates and compounds were generated using ChemDraw® Professional software version 17.1.1.0 or Collaborative Drug Discovery Inc. (CDD) CDD Vault update #3.

A Knime workflow was created to retrieve structures from an internal ChemAxon Compound Registry, generate the canonical smiles using RDKit Canon SMILES node, remove the stereochemistry using ChemAxon/Infocom MolConverter node, and name the structure using ChemAxon/Infocom Naming node. The following denotes the version of the Knime Analytics Platform and extensions utilized in the workflow:
Knime Analytics Platform 4.2.2
RDKit Knime Integration 4.0.1.v202006261025 (this extension includes the RDKit Canon SMILES node)
ChemAxon/Infocom Marvin Extensions Feature 4.3.0v202100 (this extension includes the MolConverter node)
ChemAxon/Infocom JChem Extensions Feature 4.3.0v202100 (this extension includes the Naming node)

TABLE 1A

| (G1-Z1)/(G2-Z2) # | Structure | IUPAC |
|---|---|---|
| GZ-1 | 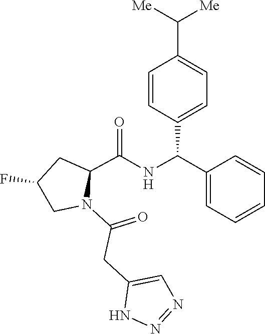 [SMILES: CC(C)c1ccc([C@@H](NC(=O)[C@@H]2C[C@@H](F)CN2C(=O)Cc2cnn[nH]2)c2ccccc2)cc1] | (2S,4R)-1-(2-(1H-1,2,3-triazol-5- yl)acetyl)-4-fluoro-N-((S)-(4- isopropylphenyl)(phenyl)methyl) pyrrolidine-2-carboxamide |
| GZ-2 | 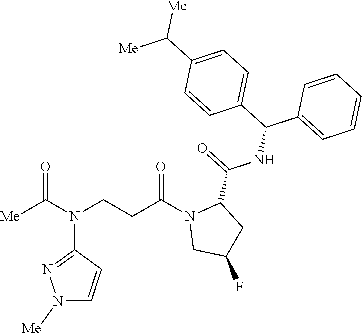 | (2S,4R)-4-fluoro-1-{3-[N-(1- methyl-1H-pyrazol-3- yl)acetamido]propanoyl} -N-[(S)- phenyl[4-(propan-2- yl)phenyl]methyl]pyrrolidine-2- carboxamide |
| GZ-3 | 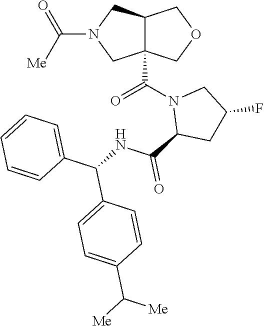 [SMILES: CC(=O)N1C[C@H]2COC[C@@]2(C(=O)N2C[C@H](F)C[C@H]2C(=O)N[C@@H](c2ccccc2)c2ccc(C(C)C)cc2)C1] | (2S,4R)-1-[(3aS,6aS)-5-acetyl- hexahydro-1H-furo[3,4-c]pyrrole- 3a-carbonyl]-4-fluoro-N-[(S)- phenyl[4-(propan-2-yl)phenyl methyl]pyrrolidine-2- carboxamide |
| GZ-4 | 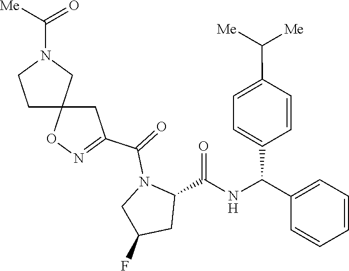 [SMILES: CC(=O)N1CCC2(CC(C(=O)N3C[C@H](F)C[C@H]3C(=O)N[C@@H](c3ccccc3)c3ccc(C(C)C)cc3)=NO2)C1] | (2S,4R)-1-{7-acetyl-1-oxa-2,7- diazaspiro[4.4]non-2-ene-3- carbonyl}-4-fluoro-N-[(S)- phenyl[4-(propan-2- yl)phenyl methyl]pyrrolidine-2- carboxamide |
| GZ-5 | 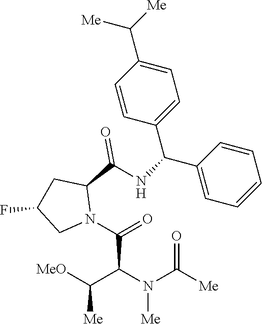 [SMILES: CO[C@H](C)[C@@H](C(=O)N1C[C@H](F)C[C@H]1C(=O)N[C@@H](c1ccccc1)c1ccc(C(C)C)cc1)N(C)C(C)=O] | (2S,4R)-4-fluoro-1-[(2S,3R)-3- methoxy-2-(N- methylacetamido)butanoyl]-N-[(S)- phenyl[4-(propan-2- yl)phenyl methyl]pyrrolidine-2- carboxamide |
| GZ-6 | 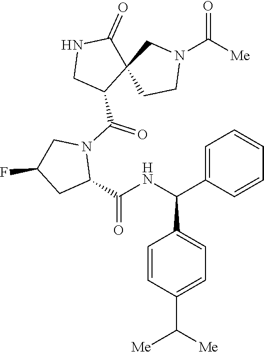 [SMILES: CC(=O)N1CC[C@@]2(C1)C(=O)NC[C@H]2C(=O)N1C[C@H](F)C[C@H]1C(=O)N[C@@H](c1ccccc1)c1ccc(C(C)C)cc1] | (2S,4R)-1-[(4S,5R)-7-acetyl-1-oxo- 2,7-diazaspiro[4.4]nonane-4- carbonyl]-4-fluoro-N-[(S)- phenyl[4-(propan-2- yl)phenyl]methyl]pyrrolidine-2- carboxamide |
| GZ-7 | 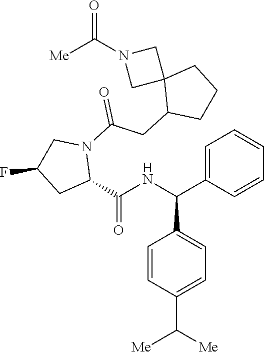 [SMILES: CC(=O)N1CC2(CCCC2CC(=O)N2C[C@H](F)C[C@H]2C(=O)N[C@@H](c2ccccc2)c2ccc(C(C)C)cc2)C1] | (2S,4R)-1-(2-{2-acetyl-2- azaspiro[3.4]octan-5-yl} acetyl)-4- fluoro-N-[(S)-phenyl[4-(propan-2- yl)phenyl methyl]pyrrolidine-2- carboxamide |
| GZ-8 | 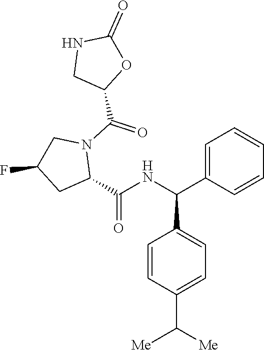 [SMILES: CC(C)c1ccc([C@@H](NC(=O)[C@@H]2C[C@@H](F)CN2C(=O)[C@@H]2CNC(=O)O2)c2ccccc2)cc1] | (2S,4R)-4-fluoro-1-[(5S)-2-oxo- 1,3-oxazolidine-5-carbonyl]-N- [(S)-phenyl[4-(propan-2- yl)phenyl\|methyl]pyrrolidine-2- carboxamide |
| GZ-9 | 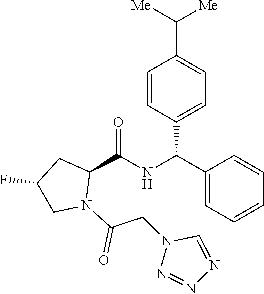 [SMILES: CC(C)c1ccc([C@@H](NC(=O)[C@@H]2C[C@@H](F)CN2C(=O)Cn2cnnn2)c2ccccc2)cc1] | (2S,4R)-4-fluoro-N-[(S)-phenyl[4- (propan-2-yl)phenyl]methyl]-1-[2- (1H-1,2,3,4-tetrazol-1- yl)acetyl]pyrrolidine-2- carboxamide |
| GZ-10 | 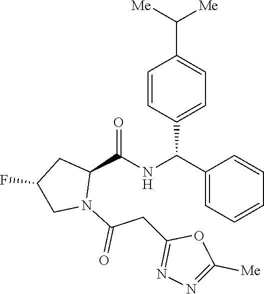 [SMILES: Cc1nnc(CC(=O)N2C[C@H](F)C[C@H]2C(=O)N[C@@H](c2ccccc2)c2ccc(C(C)C)cc2)o1] | (2S,4R)-4-fluoro-1-[2-(5-methyl- 1,3,4-oxadiazol-2-yl)acetyl]-N- [(S)-phenyl[4-(propan-2- yl)phenyl]methyl]pyrrolidine-2- carboxamide |
| GZ-11 | 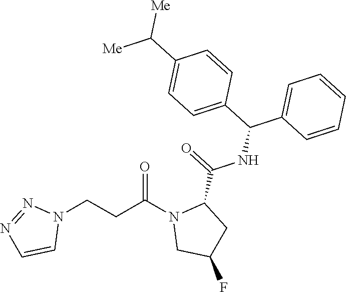 [SMILES: CC(C)c1ccc([C@@H](NC(=O)[C@@H]2C[C@@H](F)CN2C(=O)CCn2ccnn2)c2ccccc2)cc1] | (2S,4R)-4-fluoro-N-[(S)-phenyl[4- (propan-2-yl)phenyl]methyl]-1-[3- (1H-1,2,3-triazol-1- yl)propanoyl]pyrrolidine-2- carboxamide |
| GZ-12 | 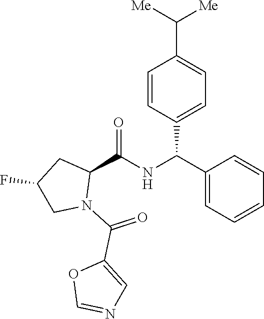 [SMILES: CC(C)c1ccc([C@@H](NC(=O)[C@@H]2C[C@@H](F)CN2C(=O)c2cnco2)c2ccccc2)cc1] | (2S,4R)-4-fluoro-1-(1,3-oxazole-5- carbonyl)-N-[(S)-phenyl[4-(propan- 2-yl)phenyl]methyl]pyrrolidine-2- carboxamide |
| GZ-13 | 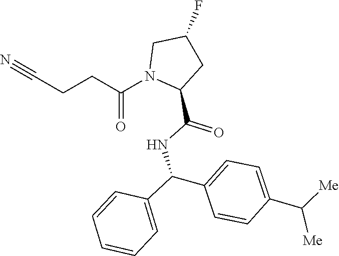 [SMILES: CC(C)c1ccc([C@@H](NC(=O)[C@@H]2C[C@@H](F)CN2C(=O)CCC#N)c2ccccc2)cc1] | (2S,4R)-1-(3-cyanopropanoyl)-4- fluoro-N-[(S)-phenyl[4-(propan-2- yl)phenyl]methyl]pyrrolidine-2- carboxamide |
| GZ-14 | 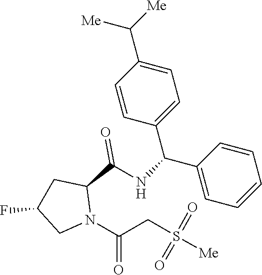 [SMILES: CC(C)c1ccc([C@@H](NC(=O)[C@@H]2C[C@@H](F)CN2C(=O)CS(C)(=O)=O)c2ccccc2)cc1] | (2S,4R)-4-fluoro-1-(2- methanesulfonylacetyl)-N-[(S)- phenyl[4-(propan-2- yl)phenyl]methyl]pyrrolidine-2- carboxamide |
| GZ-15 | 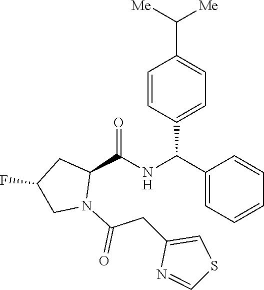 [SMILES: CC(C)c1ccc([C@@H](NC(=O)[C@@H]2C[C@@H](F)CN2C(=O)Cc2cscn2)c2ccccc2)cc1] | (2S,4R)-4-fluoro-N-[(S)-phenyl[4- (propan-2-yl)phenyl]methyl]-1-[2- (1,3-thiazol-4-yl)acetyl]pyrrolidine- 2-carboxamide |
| GZ-16 | 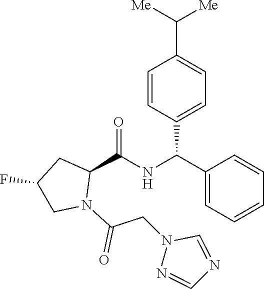 [SMILES: CC(C)c1ccc([C@@H](NC(=O)[C@@H]2C[C@@H](F)CN2C(=O)Cn2cncn2)c2ccccc2)cc1] | (2S,4R)-4-fluoro-N-[(S)-phenyl[4- (propan-2-yl)phenyl]methyl]-1-[2- (1H-1,2,4-triazol-1- yl)acetyl]pyrrolidine-2- carboxamide |
| GZ-17 | 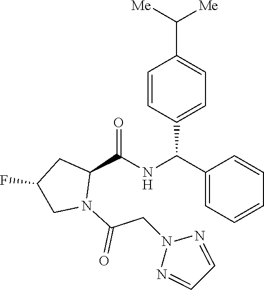 [SMILES: CC(C)c1ccc([C@@H](NC(=O)[C@@H]2C[C@@H](F)CN2C(=O)Cn2nccn2)c2ccccc2)cc1] | (2S,4R)-4-fluoro-N-[(S)-phenyl[4- (propan-2-yl)phenyl]methyl]-1-[2- (2H-1,2,3-triazol-2- yl)acetyl]pyrrolidine-2- carboxamide |
| GZ-18 | 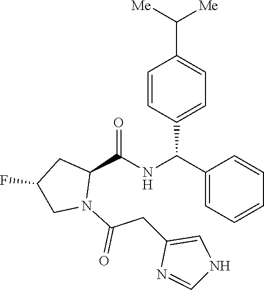 [SMILES: CC(C)c1ccc([C@@H](NC(=O)[C@@H]2C[C@@H](F)CN2C(=O)Cc2c[nH]cn2)c2ccccc2)cc1] | (2S,4R)-4-fluoro-1-[2-(1H- imidazol-4-yl)acetyl]-N-[(S)- phenyl[4-(propan-2- yl)phenyl methyl]pyrrolidine-2- carboxamide |
| GZ-19 | 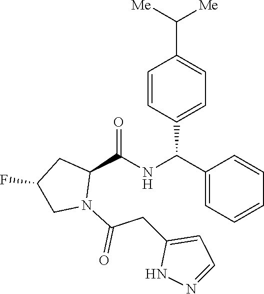 [SMILES: CC(C)c1ccc([C@@H](NC(=O)[C@@H]2C[C@@H](F)CN2C(=O)Cc2ccn[nH]2)c2ccccc2)cc1] | (2S,4R)-4-fluoro-N-[(S)-phenyl[4- (propan-2-yl)phenyl]methyl]-1-[2- (1H-pyrazol-5- yl)acetyl]pyrrolidine-2- carboxamide |
| GZ-20 | 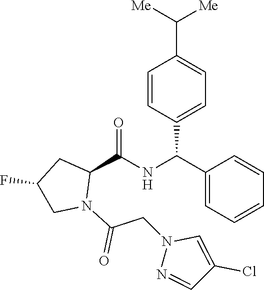 [SMILES: CC(C)c1ccc([C@@H](NC(=O)[C@@H]2C[C@@H](F)CN2C(=O)Cn2cc(Cl)cn2)c2ccccc2)cc1] | (2S,4R)-1-[2-(4-chloro-1H-pyrazol- 1-yl)acetyl]-4-fluoro-N-[(S)- phenyl[4-(propan-2- yl)phenyl]methyl]pyrrolidine-2- carboxamide |
| GZ-21 | 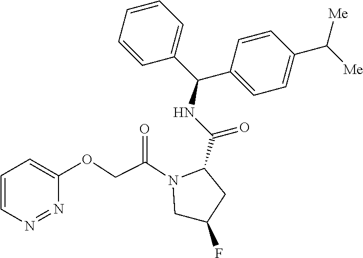 [SMILES: CC(C)c1ccc([C@@H](NC(=O)[C@@H]2C[C@@H](F)CN2C(=O)COc2cccnn2)c2ccccc2)cc1] | (2S,4R)-4-fluoro-N-[(S)-phenyl[4- (propan-2-yl)phenyl]methyl]-1-[2- (pyridazin-3- yloxy)acetyl]pyrrolidine-2- carboxamide |
| GZ-22 | 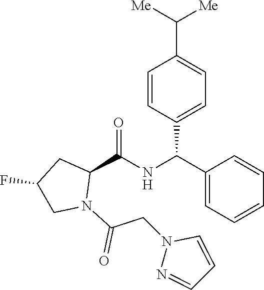 [SMILES: CC(C)c1ccc([C@@H](NC(=O)[C@@H]2C[C@@H](F)CN2C(=O)Cn2cccn2)c2ccccc2)cc1] | (2S,4R)-4-fluoro-N-[(S)-phenyl[4- (propan-2-yl)phenyl]methyl]-1-[2- (1H-pyrazol-1- yl)acetyl]pyrrolidine-2- carboxamide |
| GZ-23 | 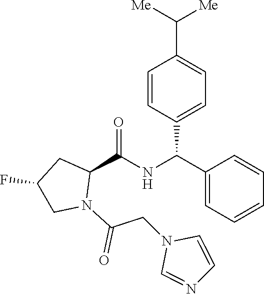 [SMILES: CC(C)c1ccc([C@@H](NC(=O)[C@@H]2C[C@@H](F)CN2C(=O)Cn2ccnc2)c2ccccc2)cc1] | (2S,4R)-4-fluoro-1-[2-(1H- imidazol-1-yl)acetyl]-N-[(S)- phenyl[4-(propan-2- yl)phenyl methyl]pyrrolidine-2- carboxamide |
| GZ-24 | 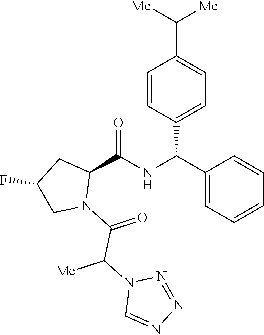 [SMILES: CC(C)c1ccc([C@@H](NC(=O)[C@@H]2C[C@@H](F)CN2C(=O)C(C)n2cnnn2)c2ccccc2)cc1] | (2S,4R)-4-fluoro-N-[(S)-phenyl[4- (propan-2-yl)phenyl]methyl]-1-[2- (1H-1,2,3,4-tetrazol-1- yl)propanoyl]pyrrolidine-2- carboxamide |
| GZ-25 | 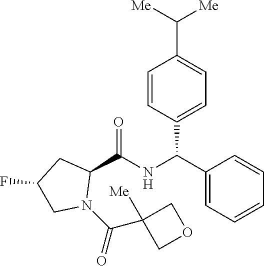 [SMILES: CC(C)c1ccc([C@@H](NC(=O)[C@@H]2C[C@@H](F)CN2C(=O)C2(C)COC2)c2ccccc2)cc1] | (2S,4R)-4-fluoro-1-(3- methyloxetane-3-carbonyl)-N-[(S)- phenyl[4-(propan-2- yl)phenyl]methyl]pyrrolidine-2- carboxamide |
| GZ-26 | 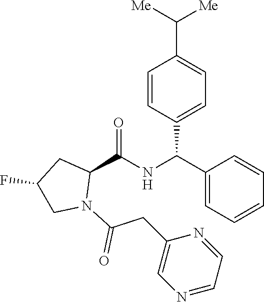 [SMILES: CC(C)c1ccc([C@@H](NC(=O)[C@@H]2C[C@@H](F)CN2C(=O)Cc2cnccn2)c2ccccc2)cc1] | (2S,4R)-4-fluoro-N-[(S)-phenyl[4- (propan-2-yl)phenyl]methyl]-1-[2- (pyrazin-2-yl)acetyl]pyrrolidine-2- carboxamide |
| GZ-27 | 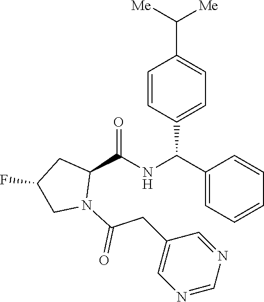 [SMILES: CC(C)c1ccc([C@@H](NC(=O)[C@@H]2C[C@@H](F)CN2C(=O)Cc2cncnc2)c2ccccc2)cc1] | (2S,4R)-4-fluoro-N-[(S)-phenyl[4- (propan-2-yl)phenyl]methyl]-1-[2- (pyrimidin-5-yl)acetyl]pyrrolidine- 2-carboxamide |
| GZ-28 | 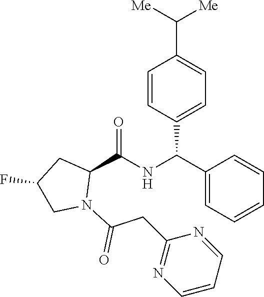 [SMILES: CC(C)c1ccc([C@@H](NC(=O)[C@@H]2C[C@@H](F)CN2C(=O)Cc2ncccn2)c2ccccc2)cc1] | (2S,4R)-4-fluoro-N-[(S)-phenyl[4- (propan-2-yl)phenyl]methyl]-1-[2- (pyrimidin-2-yl)acetyl]pyrrolidine- 2-carboxamide |
| GZ-29 | 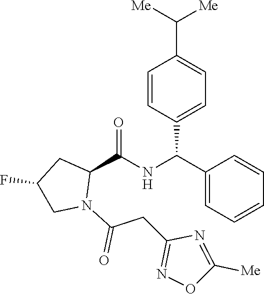 [SMILES: Cc1nc(CC(=O)N2C[C@H](F)C[C@H]2C(=O)N[C@@H](c2ccccc2)c2ccc(C(C)C)cc2)no1] | (2S,4R)-4-fluoro-1-[2-(5-methyl- 1,2,4-oxadiazol-3-yl)acetyl]-N- [(S)-phenyl[4-(propan-2- yl)phenyl methyl]pyrrolidine-2- carboxamide |
| GZ-30 | 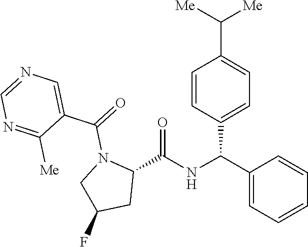 [SMILES: Cc1ncncc1C(=O)N1C[C@H](F)C[C@H]1C(=O)N[C@@H](c1ccccc1)c1ccc(C(C)C)cc1] | (2S,4R)-4-fluoro-1-(4- methylpyrimidine-5-carbonyl)-N- [(S)-phenyl[4-(propan-2- yl)phenyl]methyl]pyrrolidine-2- carboxamide |
| GZ-31 | 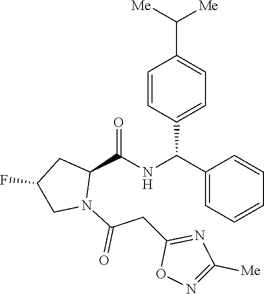 [SMILES: Cc1noc(CC(=O)N2C[C@H](F)C[C@H]2C(=O)N[C@@H](c2ccccc2)c2ccc(C(C)C)cc2)n1] | (2S,4R)-4-fluoro-1-[2-(3-methyl- 1,2,4-oxadiazol-5-yl)acetyl]-N- [(S)-phenyl[4-(propan-2- yl)phenyl methyl]pyrrolidine-2- carboxamide |
| GZ-32 | 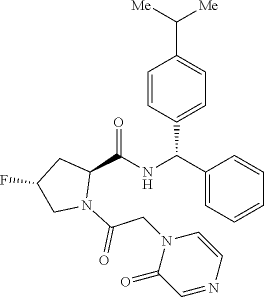 [SMILES: CC(C)c1ccc([C@@H](NC(=O)[C@@H]2C[C@@H](F)CN2C(=O)Cn2ccncc2=O)c2ccccc2)cc1] | (2S,4R)-4-fluoro-1-[2-(2-oxo-1,2- dihydropyrazin-1-yl)acetyl]-N-[(S)- phenyl[4-(propan-2- yl)phenyl]methyl]pyrrolidine-2- carboxamide |
| GZ-33 | 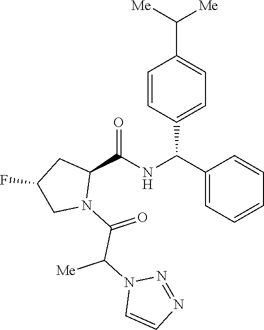 [SMILES: CC(C)c1ccc([C@@H](NC(=O)[C@@H]2C[C@@H](F)CN2C(=O)C(C)n2ccnn2)c2ccccc2)cc1] | (2S,4R)-4-fluoro-N-[(S)-phenyl[4- (propan-2-yl)phenyl methyl]-1-[2- (1H-1,2,3-triazol-1- yl)propanoyl]pyrrolidine-2- carboxamide |
| GZ-34 | 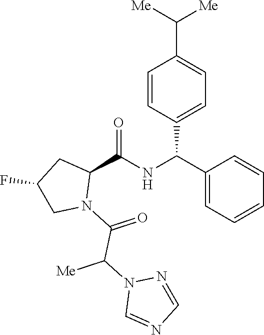 [SMILES: CC(C)c1ccc([C@@H](NC(=O)[C@@H]2C[C@@H](F)CN2C(=O)C(C)n2cncn2)c2ccccc2)cc1] | (2S,4R)-4-fluoro-N-[(S)-phenyl[4- (propan-2-yl)phenyl]methyl]-1-[2- (1H-1,2,4-triazol-1- yl)propanoyl]pyrrolidine-2- carboxamide |
| GZ-35 | 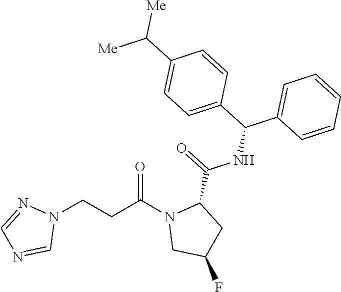 [SMILES: CC(C)c1ccc([C@@H](NC(=O)[C@@H]2C[C@@H](F)CN2C(=O)CCn2cncn2)c2ccccc2)cc1] | (2S,4R)-4-fluoro-N-[(S)-phenyl[4- (propan-2-yl)phenyl]methyl]-1-[3- (1H-1,2,4-triazol-1- yl)propanoyl]pyrrolidine-2- carboxamide |
| GZ-36 | 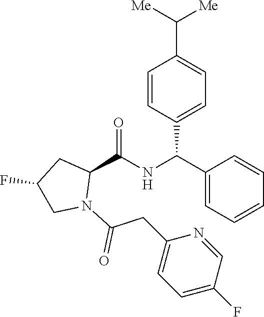 [SMILES: CC(C)c1ccc([C@@H](NC(=O)[C@@H]2C[C@@H](F)CN2C(=O)Cc2ccc(F)cn2)c2ccccc2)cc1] | (2S,4R)-4-fluoro-1-[2-(5- fluoropyridin-2-yl)acetyl]-N-[(S)- phenyl[4-(propan-2- yl)phenyl]methyl]pyrrolidine-2- carboxamide |
| GZ-37 | 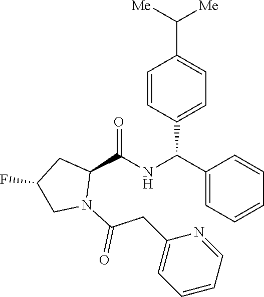 [SMILES: CC(C)c1ccc([C@@H](NC(=O)[C@@H]2C[C@@H](F)CN2C(=O)Cc2ccccn2)c2ccccc2)cc1] | (2S,4R)-4-fluoro-N-[(S)-phenyl[4- (propan-2-yl)phenyl]methyl]-1-[2- (pyridin-2-yl)acetyl]pyrrolidine-2- carboxamide |
| GZ-38 | 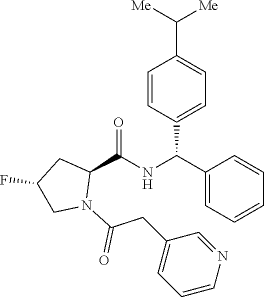 [SMILES: CC(C)c1ccc([C@@H](NC(=O)[C@@H]2C[C@@H](F)CN2C(=O)Cc2cccnc2)c2ccccc2)cc1] | (2S,4R)-4-fluoro-N-[(S)-phenyl[4- (propan-2-yl)phenyl]methyl]-1-[2- (pyridin-3-yl)acetyl]pyrrolidine-2- carboxamide |
| GZ-39 | 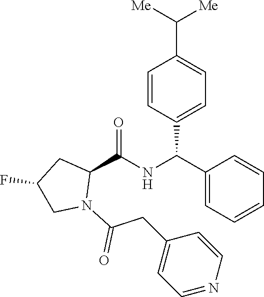 [SMILES: CC(C)c1ccc([C@@H](NC(=O)[C@@H]2C[C@@H](F)CN2C(=O)Cc2ccncc2)c2ccccc2)cc1] | (2S,4R)-4-fluoro-N-[(S)-phenyl[4- (propan-2-yl)phenyl]methyl]-1-[2- (pyridin-4-yl)acetyl]pyrrolidine-2- carboxamide |
| GZ-40 | 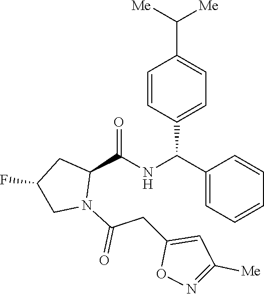 [SMILES: Cc1cc(CC(=O)N2C[C@H](F)C[C@H]2C(=O)N[C@@H](c2ccccc2)c2ccc(C(C)C)cc2)on1] | (2S,4R)-4-fluoro-1-[2-(3-methyl- 1,2-oxazol-5-yl)acetyl]-N-[(S)- phenyl[4-(propan-2- yl)phenyl]methyl]pyrrolidine-2- carboxamide |
| GZ-41 | 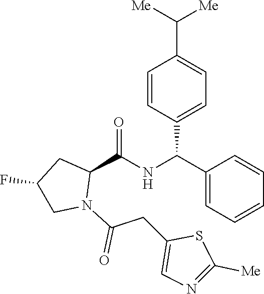 [SMILES: Cc1ncc(CC(=O)N2C[C@H](F)C[C@H]2C(=O)N[C@@H](c2ccccc2)c2ccc(C(C)C)cc2)s1] | (2S,4R)-4-fluoro-1-[2-(2-methyl- 1,3-thiazol-5-yl)acetyl]-N-[(S)- phenyl[4-(propan-2- yl)phenyl]methyl]pyrrolidine-2- carboxamide |
| GZ-42 | 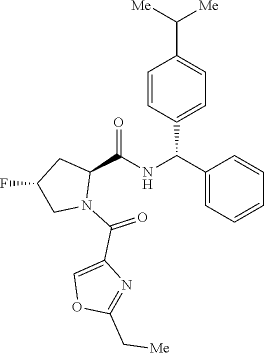 [SMILES: CCc1nc(C(=O)N2C[C@H](F)C[C@H]2C(=O)N[C@@H](c2ccccc2)c2ccc(C(C)C)cc2)co1] | (2S,4R)-1-(2-ethyl-1,3-oxazole-4- carbonyl)-4-fluoro-N-[(S)- phenyl[4-(propan-2- yl)phenyl methyl]pyrrolidine-2- carboxamide |
| GZ-43 | 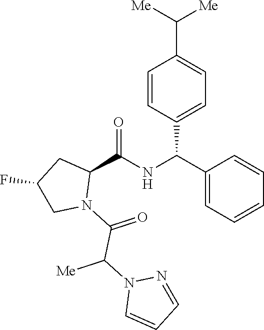 [SMILES: CC(C)c1ccc([C@@H](NC(=O)[C@@H]2C[C@@H](F)CN2C(=O)C(C)n2cccn2)c2ccccc2)cc1] | (2S,4R)-4-fluoro-N-[(S)-phenyl[4- (propan-2-yl)phenyl]methyl]-1-[2- (1H-pyrazol-1- yl)propanoyl]pyrrolidine-2- carboxamide |
| GZ-44 | 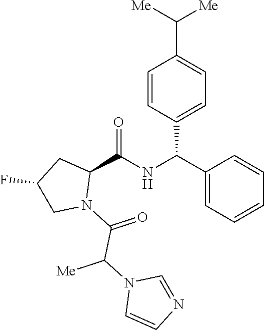 [SMILES: CC(C)c1ccc([C@@H](NC(=O)[C@@H]2C[C@@H](F)CN2C(=O)C(C)n2ccnc2)c2ccccc2)cc1] | (2S,4R)-4-fluoro-1-[2-(1H- imidazol-1-yl)propanoyl]-N-[(S)- phenyl[4-(propan-2- yl)phenyl]methyl]pyrrolidine-2- carboxamide |
| GZ-45 | 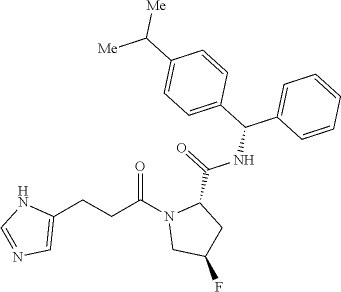 [SMILES: CC(C)c1ccc([C@@H](NC(=O)[C@@H]2C[C@@H](F)CN2C(=O)CCc2cnc[nH]2)c2ccccc2)cc1] | (2S,4R)-4-fluoro-1-[3-(1H- imidazol-5-yl)propanoyl]-N-[(S)- phenyl[4-(propan-2- yl)phenyl]methyl]pyrrolidine-2- carboxamide |
| GZ-46 | 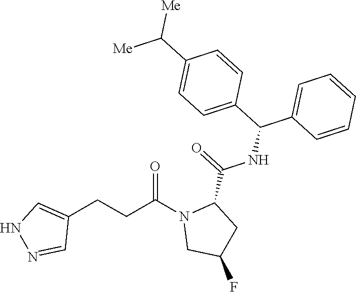 [SMILES: CC(C)c1ccc([C@@H](NC(=O)[C@@H]2C[C@@H](F)CN2C(=O)CCc2cn[nH]c2)c2ccccc2)cc1] | (2S,4R)-4-fluoro-N-[(S)-phenyl[4- (propan-2-yl)phenyl]methyl]-1-[3- (1H-pyrazol-4- yl)propanoyl]pyrrolidine-2- carboxamide |
| GZ-47 | 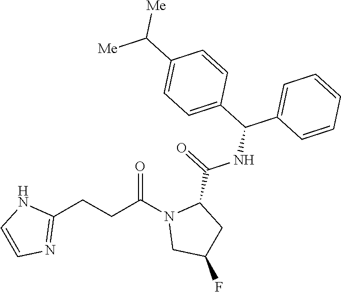 [SMILES: CC(C)c1ccc([C@@H](NC(=O)[C@@H]2C[C@@H](F)CN2C(=O)CCc2ncc[nH]2)c2ccccc2)cc1] | (2S,4R)-4-fluoro-1-[3-(1H- imidazol-2-yl)propanoyl]-N-[(S)- phenyl[4-(propan-2- yl)phenyl]methyl]pyrrolidine-2- carboxamide |
| GZ-48 | 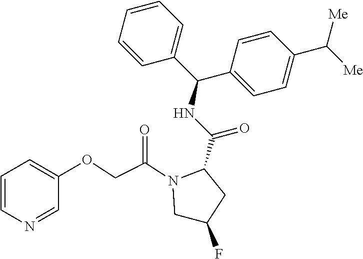 [SMILES: CC(C)c1ccc([C@@H](NC(=O)[C@@H]2C[C@@H](F)CN2C(=O)COc2cccnc2)c2ccccc2)cc1] | (2S,4R)-4-fluoro-N-[(S)-phenyl[4- (propan-2-yl)phenyl]methyl]-1-[2- (pyridin-3- yloxy)acetyl]pyrrolidine-2- carboxamide |
| GZ-49 | 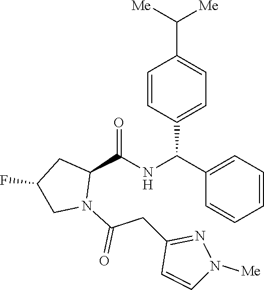 [SMILES: CC(C)c1ccc([C@@H](NC(=O)[C@@H]2C[C@@H](F)CN2C(=O)Cc2ccn(C)n2)c2ccccc2)cc1] | (2S,4R)-4-fluoro-1-[2-(1-methyl- 1H-pyrazol-3-yl)acetyl]-N-[(S)- phenyl[4-(propan-2- yl)phenyl\|methyl]pyrrolidine-2- carboxamide |
| GZ-50 | 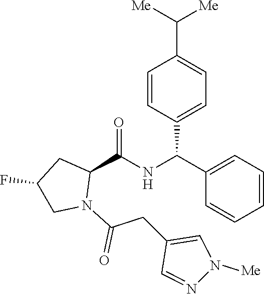 [SMILES: CC(C)c1ccc([C@@H](NC(=O)[C@@H]2C[C@@H](F)CN2C(=O)Cc2cnn(C)c2)c2ccccc2)cc1] | (2S,4R)-4-fluoro-1-[2-(1-methyl- 1H-pyrazol-4-yl)acetyl]-N-[(S)- phenyl[4-(propan-2- yl)phenyl]methyl]pyrrolidine-2- carboxamide |
| GZ-51 | 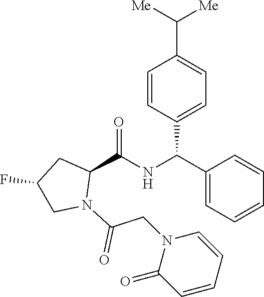 [SMILES: CC(C)c1ccc([C@@H](NC(=O)[C@@H]2C[C@@H](F)CN2C(=O)Cn2ccccc2=O)c2ccccc2)cc1] | (2S,4R)-4-fluoro-1-[2-(2-oxo-1,2- dihydropyridin-1-yl)acetyl]-N-[(S)- phenyl[4-(propan-2- yl)phenyl]methyl]pyrrolidine-2- carboxamide |
| GZ-52 | 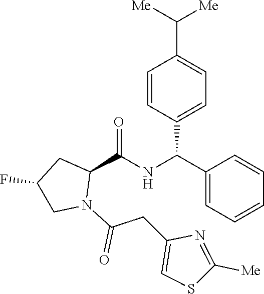 [SMILES: Cc1nc(CC(=O)N2C[C@H](F)C[C@H]2C(=O)N[C@@H](c2ccccc2)c2ccc(C(C)C)cc2)cs1] | (2S,4R)-4-fluoro-1-[2-(2-methyl- 1,3-thiazol-4-yl)acetyl]-N-[(S)- phenyl[4-(propan-2- yl)phenyl]methyl]pyrrolidine-2- carboxamide |
| GZ-53 | 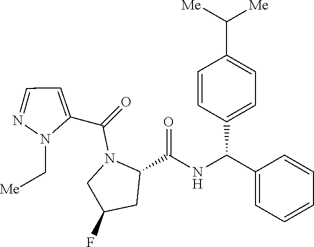 [SMILES: CCn1nccc1C(=O)N1C[C@H](F)C[C@H]1C(=O)N[C@@H](c1ccccc1)c1ccc(C(C)C)cc1] | (2S,4R)-1-(1-ethyl-1H-pyrazole-5- carbonyl)-4-fluoro-N-[(S)- phenyl[4-(propan-2- yl)phenyl]methyl]pyrrolidine-2- carboxamide |
| GZ-54 | 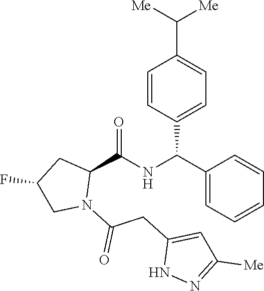 [SMILES: Cc1cc(CC(=O)N2C[C@H](F)C[C@H]2C(=O)N[C@@H](c2ccccc2)c2ccc(C(C)C)cc2)[nH]n1] | (2S,4R)-4-fluoro-1-[2-(3-methyl- 1H-pyrazol-5-yl)acetyl]-N-[(S)- phenyl[4-(propan-2- yl)phenyl\|methyl]pyrrolidine-2- carboxamide |
| GZ-55 | 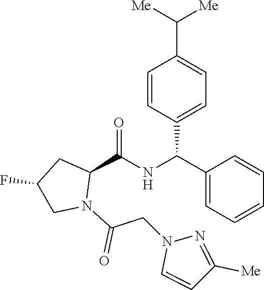 [SMILES: Cc1ccn(CC(=O)N2C[C@H](F)C[C@H]2C(=O)N[C@@H](c2ccccc2)c2ccc(C(C)C)cc2)n1] | (2S,4R)-4-fluoro-1-[2-(3-methyl- 1H-pyrazol-1-yl)acetyl]-N-[(S)- phenyl[4-(propan-2- yl)phenyl methyl]pyrrolidine-2- carboxamide |
| GZ-56 | 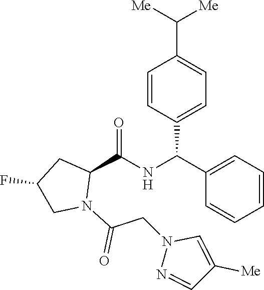 [SMILES: Cc1cnn(CC(=O)N2C[C@H](F)C[C@H]2C(=O)N[C@@H](c2ccccc2)c2ccc(C(C)C)cc2)c1] | (2S,4R)-4-fluoro-1-[2-(4-methyl- 1H-pyrazol-1-yl)acetyl]-N-[(S)- phenyl[4-(propan-2- yl)phenyl\|methyl]pyrrolidine-2- carboxamide |
| GZ-57 | 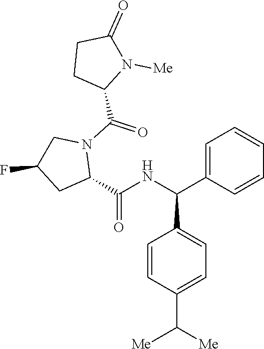 [SMILES: CC(C)c1ccc([C@@H](NC(=O)[C@@H]2C[C@@H](F)CN2C(=O)[C@@H]2CCC(=O)N2C)c2ccccc2)cc1] | (2S,4R)-4-fluoro-1-[(2S)-1-methyl- 5-oxopyrrolidine-2-carbonyl]-N- [(S)-phenyl[4-(propan-2- yl)phenyl]methyl]pyrrolidine-2- carboxamide |
| GZ-58 | 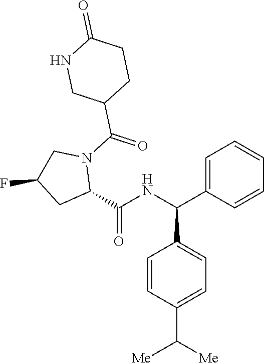 [SMILES: CC(C)c1ccc([C@@H](NC(=O)[C@@H]2C[C@@H](F)CN2C(=O)C2CCC(=O)NC2)c2ccccc2)cc1] | (2S,4R)-4-fluoro-1-(6- oxopiperidine-3-carbonyl)-N-[(S)- phenyl[4-(propan-2- yl)phenyl methyl]pyrrolidine-2- carboxamide |
| GZ-59 | 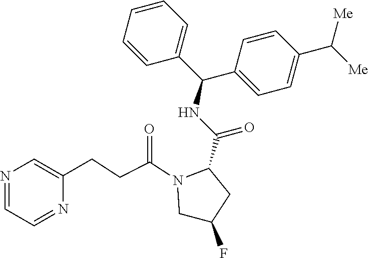 [SMILES: CC(C)c1ccc([C@@H](NC(=O)[C@@H]2C[C@@H](F)CN2C(=O)CCc2cnccn2)c2ccccc2)cc1] | (2S,4R)-4-fluoro-N-[(S)-phenyl[4- (propan-2-yl)phenyl]methyl]-1-[3- (pyrazin-2- yl)propanoyl]pyrrolidine-2- carboxamide |
| GZ-60 | 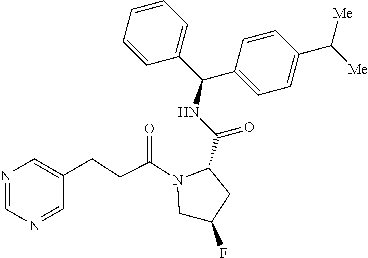 [SMILES: CC(C)c1ccc([C@@H](NC(=O)[C@@H]2C[C@@H](F)CN2C(=O)CCc2cncnc2)c2ccccc2)cc1] | (2S,4R)-4-fluoro-N-[(S)-phenyl[4- (propan-2-yl)phenyl\|methyl]-1-[3- (pyrimidin-5- yl)propanoyl]pyrrolidine-2- carboxamide |
| GZ-61 | 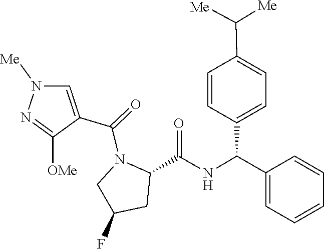 [SMILES: COc1nn(C)cc1C(=O)N1C[C@H](F)C[C@H]1C(=O)N[C@@H](c1ccccc1)c1ccc(C(C)C)cc1] | (2S,4R)-4-fluoro-1-(3-methoxy-1- methyl-1H-pyrazole-4-carbonyl)- N-[(S)-phenyl[4-(propan-2- yl)phenyl]methyl]pyrrolidine-2- carboxamide |
| GZ-62 | 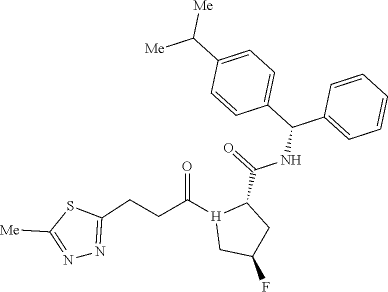 | (2S,4R)-4-fluoro-1-[3-(5-methyl- 1,3,4-thiadiazol-2-yl)propanoyl]-N- [(S)-phenyl[4-(propan-2- yl)phenyl]methyl]pyrrolidine-2- carboxamide |
| GZ-63 | 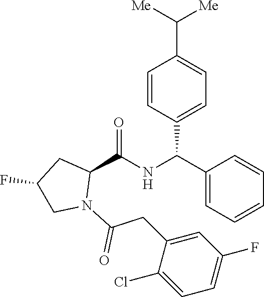 [SMILES: CC(C)c1ccc([C@@H](NC(=O)[C@@H]2C[C@@H](F)CN2C(=O)Cc2cc(F)ccc2Cl)c2ccccc2)cc1] | (2S,4R)-1-[2-(2-chloro-5- fluorophenyl)acetyl]-4-fluoro-N- [(S)-phenyl[4-(propan-2- yl)phenyl]methyl]pyrrolidine-2- carboxamide |
| GZ-64 | 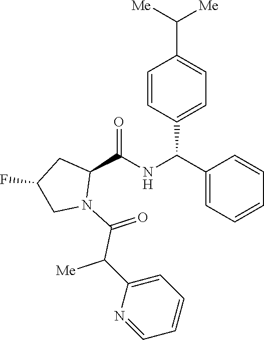 [SMILES: CC(C)c1ccc([C@@H](NC(=O)[C@@H]2C[C@@H](F)CN2C(=O)C(C)c2ccccn2)c2ccccc2)cc1] | (2S,4R)-4-fluoro-N-[(S)-phenyl[4- (propan-2-yl)phenyl]methyl]-1-[2- (pyridin-2- yl)propanoyl]pyrrolidine-2- carboxamide |
| GZ-65 | 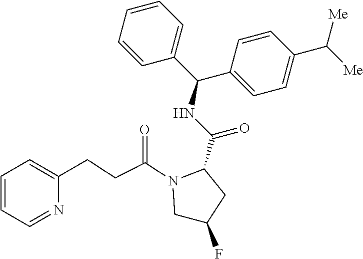 [SMILES: CC(C)c1ccc([C@@H](NC(=O)[C@@H]2C[C@@H](F)CN2C(=O)CCc2ccccn2)c2ccccc2)cc1] | (2S,4R)-4-fluoro-N-[(S)-phenyl[4- (propan-2-yl)phenyl]methyl]-1-[3- (pyridin-2- yl)propanoyl]pyrrolidine-2- carboxamide |
| GZ-66 | 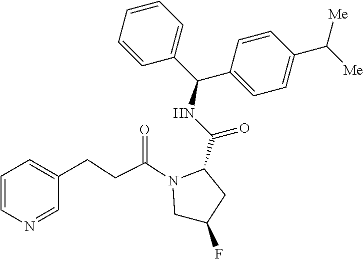 [SMILES: CC(C)c1ccc([C@@H](NC(=O)[C@@H]2C[C@@H](F)CN2C(=O)CCc2cccnc2)c2ccccc2)cc1] | (2S,4R)-4-fluoro-N-[(S)-phenyl[4- (propan-2-yl)phenyl]methyl]-1-[3- (pyridin-3- yl)propanoyl]pyrrolidine-2- carboxamide |
| GZ-67 | 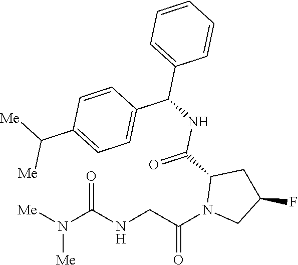 [SMILES: CC(C)c1ccc([C@@H](NC(=O)[C@@H]2C[C@@H](F)CN2C(=O)CNC(=O)N(C)C)c2ccccc2)cc1] | (2S,4R)-1-{2- [(dimethylcarbamoyl)amino]acetyl}- 4-fluoro-N-[(S)-phenyl[4-(propan- 2-yl)phenyl]methyl]pyrrolidine-2- carboxamide |
| GZ-68 | 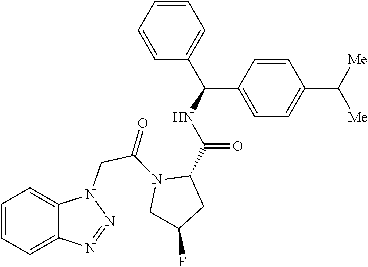 [SMILES: CC(C)c1ccc([C@@H](NC(=O)[C@@H]2C[C@@H](F)CN2C(=O)Cn2nnc3ccccc32)c2ccccc2)cc1] | (2S,4R)-1-[2-(1H-1,2,3- benzotriazol-1-yl)acetyl]-4-fluoro- N-[(S)-phenyl[4-(propan-2- yl)phenyl methyl]pyrrolidine-2- carboxamide |
| GZ-69 | 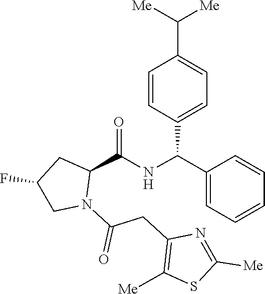 [SMILES: Cc1nc(CC(=O)N2C[C@H](F)C[C@H]2C(=O)N[C@@H](c2ccccc2)c2ccc(C(C)C)cc2)c(C)s1] | (2S,4R)-1-[2-(2,5-dimethyl-1,3- thiazol-4-yl)acetyl]-4-fluoro-N- [(S)-phenyl[4-(propan-2- yl)phenyl\|methyl]pyrrolidine-2- carboxamide |
| GZ-70 | 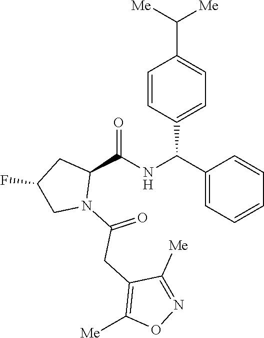 [SMILES: Cc1noc(C)c1CC(=O)N1C[C@H](F)C[C@H]1C(=O)N[C@@H](c1ccccc1)c1ccc(C(C)C)cc1] | (2S,4R)-1-[2-(3,5-dimethyl-1,2- oxazol-4-yl)acetyl]-4-fluoro-N- [(S)-phenyl[4-(propan-2- yl)phenyl methyl]pyrrolidine-2- carboxamide |
| GZ-71 | 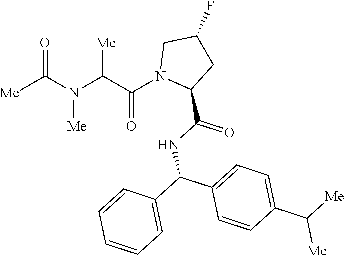 [SMILES: CC(=O)N(C)C(C)C(=O)N1C[C@H](F)C[C@H]1C(=O)N[C@@H](c1ccccc1)c1ccc(C(C)C)cc1] | (2S,4R)-4-fluoro-1-[2-(N- methylacetamido)propanoyl]-N- [(S)-phenyl[4-(propan-2- yl)phenyl]methyl]pyrrolidine-2- carboxamide |
| GZ-72 | 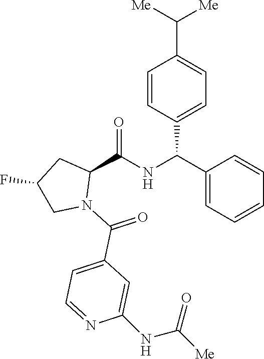 [SMILES: CC(=O)Nc1cc(C(=O)N2C[C@H](F)C[C@H]2C(=O)N[C@@H](c2ccccc2)c2ccc(C(C)C)cc2)ccn1] | (2S,4R)-1-(2-acetamidopyridine-4- carbonyl)-4-fluoro-N-[(S)- phenyl[4-(propan-2- yl)phenyl]methyl]pyrrolidine-2- carboxamide |
| GZ-73 | 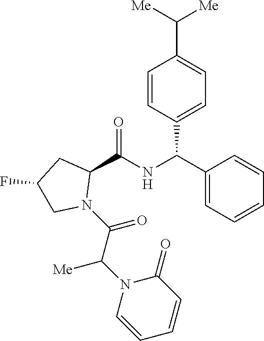 [SMILES: CC(C)c1ccc([C@@H](NC(=O)[C@@H]2C[C@@H](F)CN2C(=O)C(C)n2ccccc2=O)c2ccccc2)cc1] | (2S,4R)-4-fluoro-1-[2-(2-oxo-1,2- dihydropyridin-1-yl)propanoyl]-N- [(S)-phenyl[4-(propan-2- yl)phenyl methyl]pyrrolidine-2- carboxamide |
| GZ-74 | 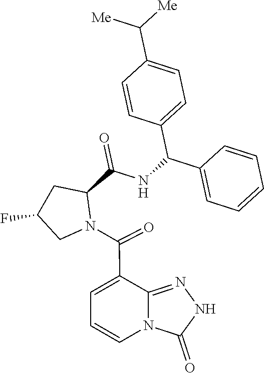 | (2S,4R)-4-fluoro-1-{3-oxo-2H,3H- [1,2,4]triazolo[4,3-a]pyridine-8- carbonyl}-N-[(S)-phenyl[4- (propan-2- yl)phenyl]methyl]pyrrolidine-2- carboxamide |
| GZ-75 | 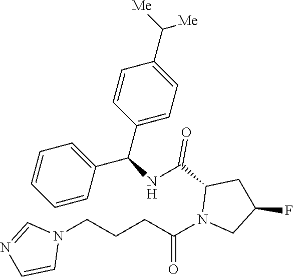 [SMILES: CC(C)c1ccc([C@@H](NC(=O)[C@@H]2C[C@@H](F)CN2C(=O)CCCn2ccnc2)c2ccccc2)cc1] | (2S,4R)-4-fluoro-1-[4-(1H- imidazol-1-yl)butanoyl]-N-[(S)- phenyl[4-(propan-2- yl)phenyl\|methyl]pyrrolidine-2- carboxamide |
| GZ-76 | 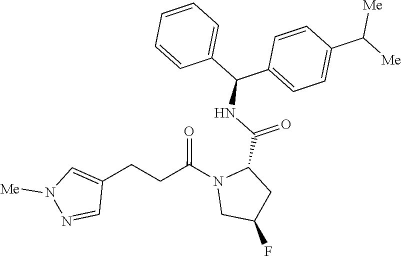 [SMILES: CC(C)c1ccc([C@@H](NC(=O)[C@@H]2C[C@@H](F)CN2C(=O)CCc2cnn(C)c2)c2ccccc2)cc1] | (2S,4R)-4-fluoro-1-[3-(1-methyl- 1H-pyrazol-4-yl)propanoyl]-N- [(S)-phenyl[4-(propan-2- yl)phenyl]methyl]pyrrolidine-2- carboxamide |
| GZ-77 | 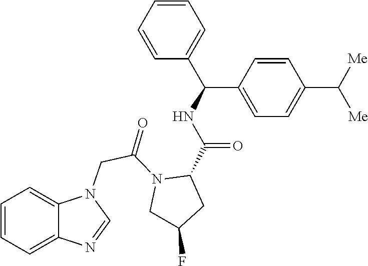 [SMILES: CC(C)c1ccc([C@@H](NC(=O)[C@@H]2C[C@@H](F)CN2C(=O)Cn2cnc3ccccc32)c2ccccc2)cc1] | (2S,4R)-1-[2-(1H-1,3-benzodiazol- 1-yl)acetyl]-4-fluoro-N-[(S)- phenyl[4-(propan-2- yl)phenyl]methyl]pyrrolidine-2- carboxamide |
| GZ-78 | 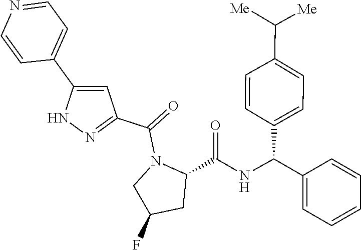 [SMILES: CC(C)c1ccc([C@@H](NC(=O)[C@@H]2C[C@@H](F)CN2C(=O)c2cc(-c3ccncc3)[nH]n2)c2ccccc2)cc1] | (2S,4R)-4-fluoro-N-[(S)-phenyl[4- (propan-2-yl)phenyl methyl]-1-[5- (pyridin-4-yl)-1H-pyrazole-3- carbonyl]pyrrolidine-2- carboxamide |
| GZ-79 | 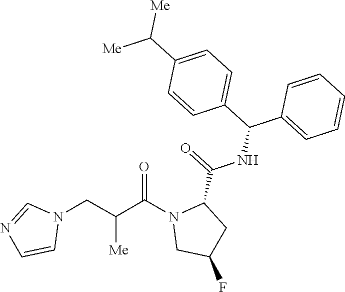 [SMILES: CC(Cn1ccnc1)C(=O)N1C[C@H](F)C[C@H]1C(=O)N[C@@H](c1ccccc1)c1ccc(C(C)C)cc1] | (2S,4R)-4-fluoro-1-[3-(1H- imidazol-1-yl)-2-methylpropanoyl]- N-[(S)-phenyl[4-(propan-2- yl)phenyl]methyl]pyrrolidine-2- carboxamide |
| GZ-80 | 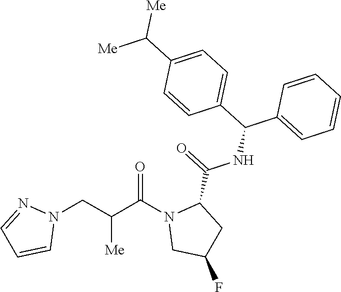 [SMILES: CC(Cn1cccn1)C(=O)N1C[C@H](F)C[C@H]1C(=O)N[C@@H](c1ccccc1)c1ccc(C(C)C)cc1] | (2S,4R)-4-fluoro-1-[2-methyl-3- (1H-pyrazol-1-yl)propanoyl]-N- [(S)-phenyl[4-(propan-2- yl)phenyl]methyl]pyrrolidine-2- carboxamide |
| GZ-81 | 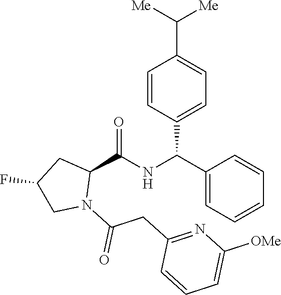 [SMILES: COc1cccc(CC(=O)N2C[C@H](F)C[C@H]2C(=O)N[C@@H](c2ccccc2)c2ccc(C(C)C)cc2)n1] | (2S,4R)-4-fluoro-1-[2-(6- methoxypyridin-2-yl)acetyl]-N- [(S)-phenyl[4-(propan-2- yl)phenyl]methyl]pyrrolidine-2- carboxamide |
| GZ-82 | 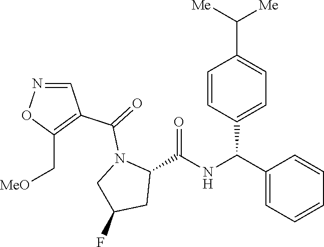 [SMILES: COCc1oncc1C(=O)N1C[C@H](F)C[C@H]1C(=O)N[C@@H](c1ccccc1)c1ccc(C(C)C)cc1] | (2S,4R)-4-fluoro-1-[5- (methoxymethyl)-1,2-oxazole-4- carbonyl]-N-[(S)-phenyl[4-(propan- 2-yl)phenyl]methyl]pyrrolidine-2- carboxamide |
| GZ-83 | 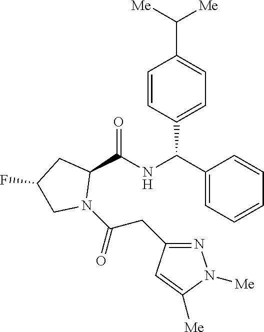 [SMILES: Cc1cc(CC(=O)N2C[C@H](F)C[C@H]2C(=O)N[C@@H](c2ccccc2)c2ccc(C(C)C)cc2)nn1C] | (2S,4R)-1-[2-(1,5-dimethyl-1H- pyrazol-3-yl)acetyl]-4-fluoro-N- [(S)-phenyl[4-(propan-2- yl)phenyl]methyl]pyrrolidine-2- carboxamide |
| GZ-84 | 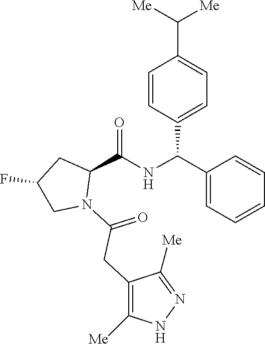 [SMILES: Cc1n[nH]c(C)c1CC(=O)N1C[C@H](F)C[C@H]1C(=O)N[C@@H](c1ccccc1)c1ccc(C(C)C)cc1] | (2S,4R)-1-[2-(3,5-dimethyl-1H- pyrazol-4-yl)acetyl]-4-fluoro-N- [(S)-phenyl[4-(propan-2- yl)phenyl]methyl]pyrrolidine-2- carboxamide |
| GZ-85 | 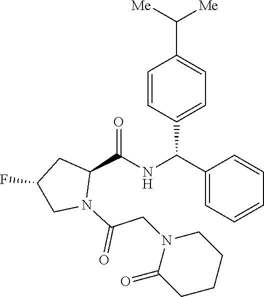 [SMILES: CC(C)c1ccc([C@@H](NC(=O)[C@@H]2C[C@@H](F)CN2C(=O)CN2CCCCC2=O)c2ccccc2)cc1] | (2S,4R)-4-fluoro-1-[2-(2- oxopiperidin-1-yl)acetyl]-N-[(S)- phenyl[4-(propan-2- yl)phenyl]methyl]pyrrolidine-2- carboxamide |
| GZ-86 | 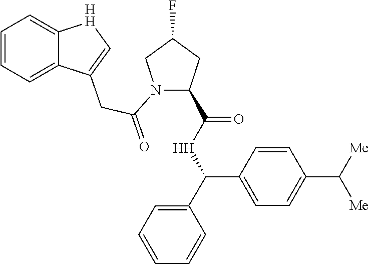 | (2S,4R)-4-fluoro-1-[2-(1H-indol-3- yl)acetyl]-N-[(S)-phenyl[4-(propan- 2-yl)phenyl]methyl]pyrrolidine-2- carboxamide |
| GZ-87 | 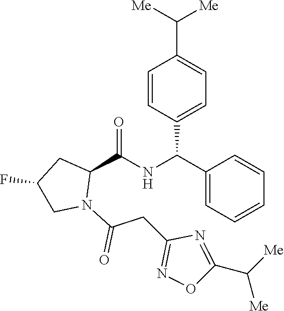 [SMILES: CC(C)c1ccc([C@@H](NC(=O)[C@@H]2C[C@@H](F)CN2C(=O)Cc2noc(C(C)C)n2)c2ccccc2)cc1] | (2S,4R)-4-fluoro-N-[(S)-phenyl[4- (propan-2-yl)phenyl]methyl]-1-{2- [5-(propan-2-yl)-1,2,4-oxadiazol-3- yl]acetyl } pyrrolidine-2- carboxamide |
| GZ-88 | 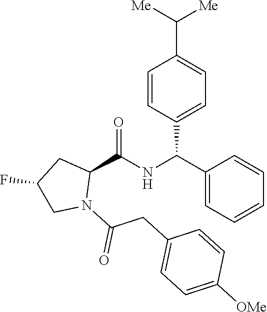 [SMILES: COc1ccc(CC(=O)N2C[C@H](F)C[C@H]2C(=O)N[C@@H](c2ccccc2)c2ccc(C(C)C)cc2)cc1] | (2S,4R)-4-fluoro-1-[2-(4- methoxyphenyl)acetyl]-N-[(S)- phenyl[4-(propan-2- yl)phenyl methyl]pyrrolidine-2- carboxamide |
| GZ-89 | 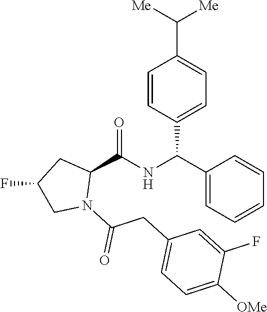 [SMILES: COc1ccc(CC(=O)N2C[C@H](F)C[C@H]2C(=O)N[C@@H](c2ccccc2)c2ccc(C(C)C)cc2)cc1F] | (2S,4R)-4-fluoro-1-[2-(3-fluoro-4- methoxyphenyl)acetyl]-N-[(S)- phenyl[4-(propan-2- yl)phenyl methyl]pyrrolidine-2- carboxamide |
| GZ-90 | 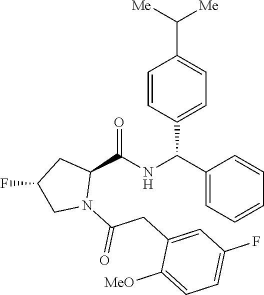 [SMILES: COc1ccc(F)cc1CC(=O)N1C[C@H](F)C[C@H]1C(=O)N[C@@H](c1ccccc1)c1ccc(C(C)C)cc1] | (2S,4R)-4-fluoro-1-[2-(5-fluoro-2- methoxyphenyl)acetyl]-N-[(S)- phenyl[4-(propan-2- yl)phenyl]methyl]pyrrolidine-2- carboxamide |
| GZ-91 | 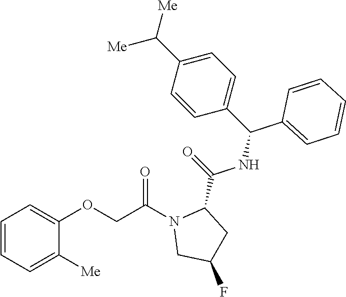 [SMILES: Cc1ccccc1OCC(=O)N1C[C@H](F)C[C@H]1C(=O)N[C@@H](c1ccccc1)c1ccc(C(C)C)cc1] | (2S,4R)-4-fluoro-1-[2-(2- methylphenoxy)acetyl]-N-[(S)- phenyl[4-(propan-2- yl)phenyl]methyl]pyrrolidine-2- carboxamide |
| GZ-92 | 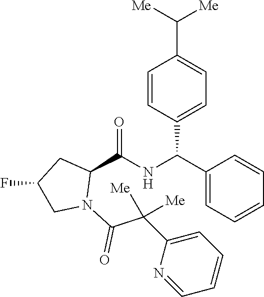 [SMILES: CC(C)c1ccc([C@@H](NC(=O)[C@@H]2C[C@@H](F)CN2C(=O)C(C)(C)c2ccccn2)c2ccccc2)cc1] | (2S,4R)-4-fluoro-1-[2-methyl-2- (pyridin-2-yl)propanoyl]-N-[(S)- phenyl[4-(propan-2- yl)phenyl]methyl]pyrrolidine-2- carboxamide |
| GZ-93 | 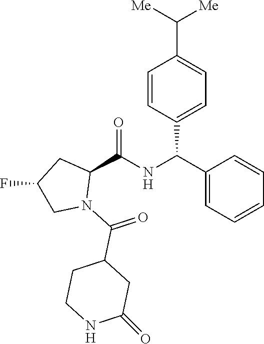 [SMILES: CC(C)c1ccc([C@@H](NC(=O)[C@@H]2C[C@@H](F)CN2C(=O)C2CCNC(=O)C2)c2ccccc2)cc1] | (2S,4R)-4-fluoro-1-(2- oxopiperidine-4-carbonyl)-N-[(S)- phenyl[4-(propan-2- yl)phenyl methyl]pyrrolidine-2- carboxamide |
| GZ-94 | 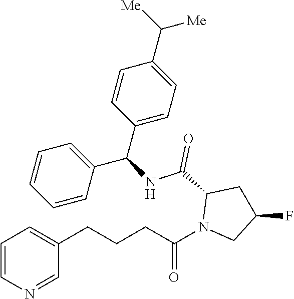 [SMILES: CC(C)c1ccc([C@@H](NC(=O)[C@@H]2C[C@@H](F)CN2C(=O)CCCc2cccnc2)c2ccccc2)cc1] | (2S,4R)-4-fluoro-N-[(S)-phenyl[4- (propan-2-yl)phenyl]methyl]-1-[4- (pyridin-3-yl)butanoyl]pyrrolidine- 2-carboxamide |
| GZ-95 | 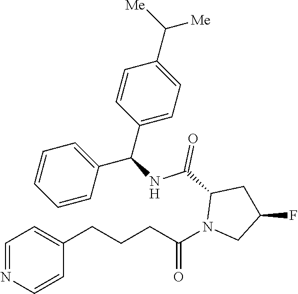 [SMILES: CC(C)c1ccc([C@@H](NC(=O)[C@@H]2C[C@@H](F)CN2C(=O)CCCc2ccncc2)c2ccccc2)cc1] | (2S,4R)-4-fluoro-N-[(S)-phenyl[4- (propan-2-yl)phenyl]methyl]-1-[4- (pyridin-4-yl)butanoyl]pyrrolidine- 2-carboxamide |
| GZ-96 | 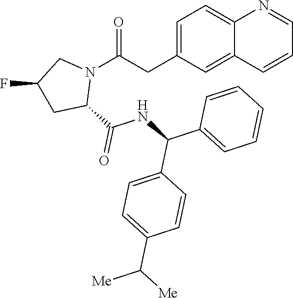 [SMILES: CC(C)c1ccc([C@@H](NC(=O)[C@@H]2C[C@@H](F)CN2C(=O)Cc2ccc3ncccc3c2)c2ccccc2)cc1] | (2S,4R)-4-fluoro-N-[(S)-phenyl[4- (propan-2-yl)phenyl]methyl]-1-[2- (quinolin-6-yl)acetyl]pyrrolidine-2- carboxamide |
| GZ-97 | 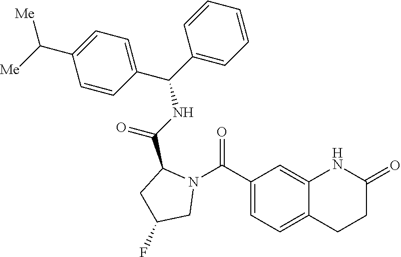 [SMILES: CC(C)c1ccc([C@@H](NC(=O)[C@@H]2C[C@@H](F)CN2C(=O)c2ccc3c(c2)NC(=O)CC3)c2ccccc2)cc1] | (2S,4R)-4-fluoro-1-(2-oxo-1,2,3,4- tetrahydroquinoline-7-carbonyl)-N- [(S)-phenyl[4-(propan-2- yl)phenyl]methyl]pyrrolidine-2- carboxamide |
| GZ-98 | 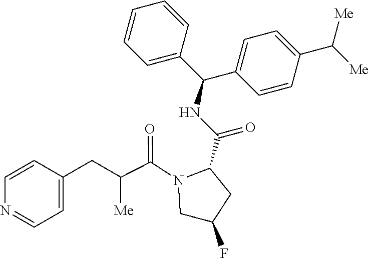 [SMILES: CC(Cc1ccncc1)C(=O)N1C[C@H](F)C[C@H]1C(=O)N[C@@H](c1ccccc1)c1ccc(C(C)C)cc1] | (2S,4R)-4-fluoro-1-[2-methyl-3- (pyridin-4-yl)propanoyl]-N-[(S)- phenyl[4-(propan-2- yl)phenyl]methyl]pyrrolidine-2- carboxamide |
| GZ-99 | 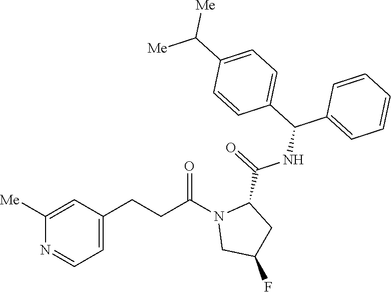 [SMILES: Cc1cc(CCC(=O)N2C[C@H](F)C[C@H]2C(=O)N[C@@H](c2ccccc2)c2ccc(C(C)C)cc2)ccn1] | (2S,4R)-4-fluoro-1-[3-(2- methylpyridin-4-yl)propanoyl]-N- [(S)-phenyl[4-(propan-2- yl)phenyl]methyl]pyrrolidine-2- carboxamide |
| GZ-100 | 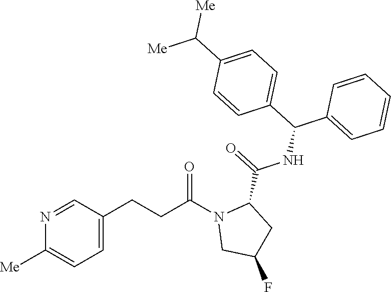 [SMILES: Cc1ccc(CCC(=O)N2C[C@H](F)C[C@H]2C(=O)N[C@@H](c2ccccc2)c2ccc(C(C)C)cc2)cn1] | (2S,4R)-4-fluoro-1-[3-(6- methylpyridin-3-yl)propanoyl]-N- [(S)-phenyl[4-(propan-2- yl)phenyl]methyl]pyrrolidine-2- carboxamide |
| GZ-101 | 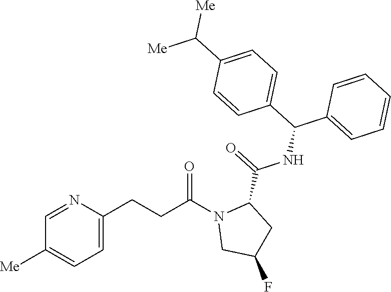 [SMILES: Cc1ccc(CCC(=O)N2C[C@H](F)C[C@H]2C(=O)N[C@@H](c2ccccc2)c2ccc(C(C)C)cc2)nc1] | (2S,4R)-4-fluoro-1-[3-(5- methylpyridin-2-yl)propanoyl]-N- [(S)-phenyl[4-(propan-2- yl)phenyl]methyl]pyrrolidine-2- carboxamide |
| GZ-102 | 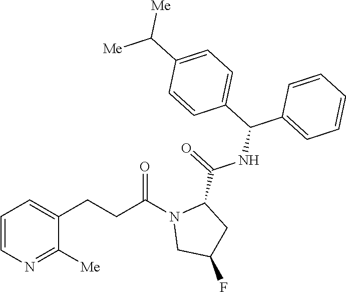 [SMILES: Cc1ncccc1CCC(=O)N1C[C@H](F)C[C@H]1C(=O)N[C@@H](c1ccccc1)c1ccc(C(C)C)cc1] | (2S,4R)-4-fluoro-1-[3-(2- methylpyridin-3-yl)propanoyl]-N- [(S)-phenyl[4-(propan-2- yl)phenylmethyl]pyrrolidine-2- carboxamide |
| GZ-103 | 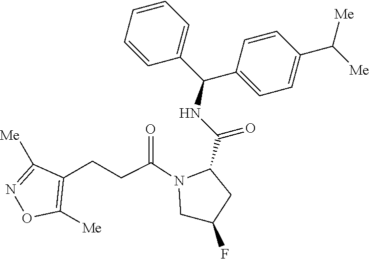 [SMILES: Cc1noc(C)c1CCC(=O)N1C[C@H](F)C[C@H]1C(=O)N[C@@H](c1ccccc1)c1ccc(C(C)C)cc1] | (2S,4R)-1-[3-(3,5-dimethyl-1,2- oxazol-4-yl)propanoyl]-4-fluoro-N- [(S)-phenyl[4-(propan-2- yl)phenyl]methyl]pyrrolidine-2- carboxamide |
| GZ-104 | 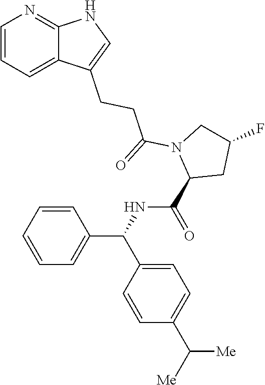 [SMILES: CC(C)c1ccc([C@@H](NC(=O)[C@@H]2C[C@@H](F)CN2C(=O)CCc2c[nH]c3ncccc23)c2ccccc2)cc1] | (2S,4R)-4-fluoro-N-[(S)-phenyl[4- (propan-2-yl)phenyl]methyl]-1-(3- {1H-pyrrolo[2,3-b]pyridin-3- yl } propanoyl)pyrrolidine-2- carboxamide |
| GZ-105 | 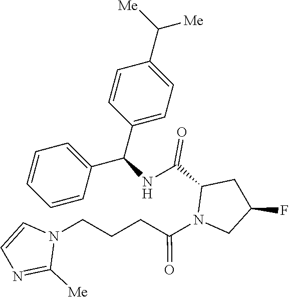 [SMILES: Cc1nccn1CCCC(=O)N1C[C@H](F)C[C@H]1C(=O)N[C@@H](c1ccccc1)c1ccc(C(C)C)cc1] | (2S,4R)-4-fluoro-1-[4-(2-methyl- 1H-imidazol-1-yl)butanoyl]-N-[(S)- phenyl[4-(propan-2- yl)phenyl]methyl]pyrrolidine-2- carboxamide |
| GZ-106 | 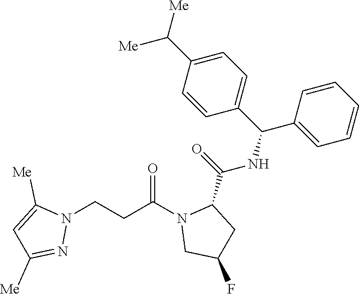 [SMILES: Cc1cc(C)n(CCC(=O)N2C[C@H](F)C[C@H]2C(=O)N[C@@H](c2ccccc2)c2ccc(C(C)C)cc2)n1] | (2S,4R)-1-[3-(3,5-dimethyl-1H- pyrazol-1-yl)propanoyl]-4-fluoro- N-[(S)-phenyl[4-(propan-2- yl)phenyl]methyl]pyrrolidine-2- carboxamide |
| GZ-107 | 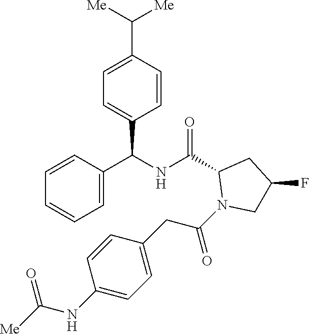 [SMILES: CC(=O)Nc1ccc(CC(=O)N2C[C@H](F)C[C@H]2C(=O)N[C@@H](c2ccccc2)c2ccc(C(C)C)cc2)cc1] | (2S,4R)-1-[2-(4- acetamidophenyl)acetyl]-4-fluoro- N-[(S)-phenyl[4-(propan-2- yl)phenyl methyl]pyrrolidine-2- carboxamide |
| GZ-108 | 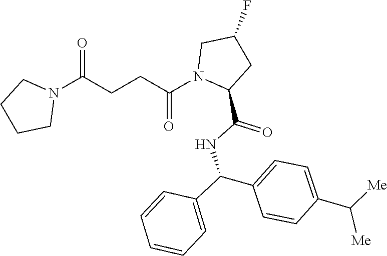 [SMILES: CC(C)c1ccc([C@@H](NC(=O)[C@@H]2C[C@@H](F)CN2C(=O)CCC(=O)N2CCCC2)c2ccccc2)cc1] | (2S,4R)-4-fluoro-1-[4-oxo-4- (pyrrolidin-1-yl)butanoyl]-N-[(S)- phenyl[4-(propan-2- yl)phenyl]methyl]pyrrolidine-2- carboxamide |
| GZ-109 | 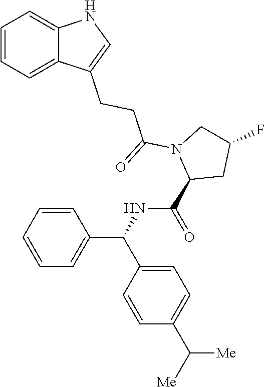 [SMILES: CC(C)c1ccc([C@@H](NC(=O)[C@@H]2C[C@@H](F)CN2C(=O)CCc2c[nH]c3ccccc23)c2ccccc2)cc1] | (2S,4R)-4-fluoro-1-[3-(1H-indol-3- yl)propanoyl]-N-[(S)-phenyl[4- (propan-2- yl)phenyl]methyl]pyrrolidine-2- carboxamide |
| GZ-110 | 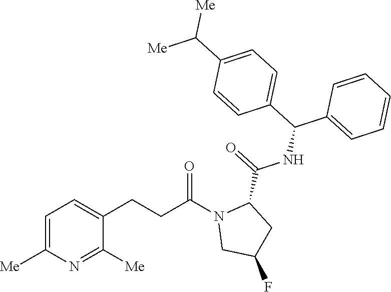 [SMILES: Cc1ccc(CCC(=O)N2C[C@H](F)C[C@H]2C(=O)N[C@@H](c2ccccc2)c2ccc(C(C)C)cc2)c(C)n1] | (2S,4R)-1-[3-(2,6-dimethylpyridin- 3-yl)propanoyl]-4-fluoro-N-[(S)- phenyl[4-(propan-2- yl)phenyl]methyl]pyrrolidine-2- carboxamide |
| GZ-111 | 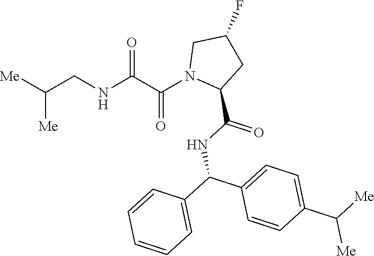 [SMILES: CC(C)CNC(=O)C(=O)N1C[C@H](F)C[C@H]1C(=O)N[C@@H](c1ccccc1)c1ccc(C(C)C)cc1] | (2S,4R)-4-fluoro-1-{[(2- methylpropyl)carbamoyl]carbonyl}- N-[(S)-phenyl[4-(propan-2- yl)phenyl methyl]pyrrolidine-2- carboxamide |
| GZ-112 | 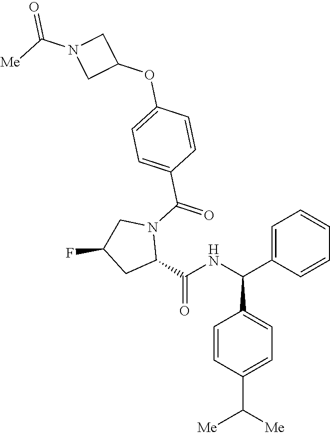 | (2S,4R)-1-{4-[(1-acetylazetidin-3- yl)oxy]benzoyl}-4-fluoro-N-[(S)- phenyl[4-(propan-2- yl)phenyl methyl]pyrrolidine-2- carboxamide |
| GZ-113 | 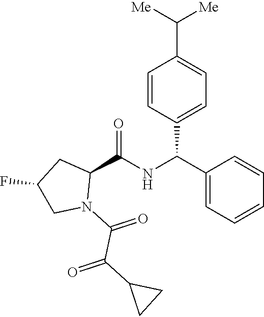 [SMILES: CC(C)c1ccc([C@@H](NC(=O)[C@@H]2C[C@@H](F)CN2C(=O)C(=O)C2CC2)c2ccccc2)cc1] | (2S,4R)-1-(2-cyclopropyl-2- oxoacetyl)-4-fluoro-N-[(S)- phenyl[4-(propan-2- yl)phenyl]methyl]pyrrolidine-2- carboxamide |
| GZ-114 | 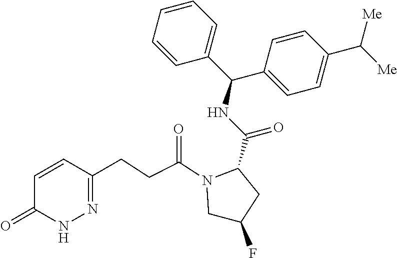 [SMILES: CC(C)c1ccc([C@@H](NC(=O)[C@@H]2C[C@@H](F)CN2C(=O)CCc2ccc(=O)[nH]n2)c2ccccc2)cc1] | (2S,4R)-4-fluoro-1-[3-(6-oxo-1,6- dihydropyridazin-3-yl)propanoyl]- N-[(S)-phenyl[4-(propan-2- yl)phenyl]methyl]pyrrolidine-2- carboxamide |
| GZ-115 | 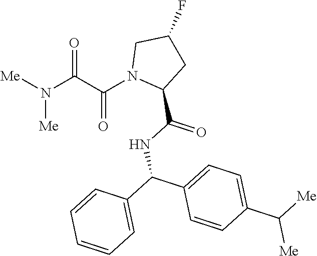 [SMILES: CC(C)c1ccc([C@@H](NC(=O)[C@@H]2C[C@@H](F)CN2C(=O)C(=O)N(C)C)c2ccccc2)cc1] | (2S,4R)-1- [(dimethylcarbamoyl)carbonyl]-4- fluoro-N-[(S)-phenyl[4-(propan-2- yl)phenyl]methyl]pyrrolidine-2- carboxamide |
| GZ-116 | 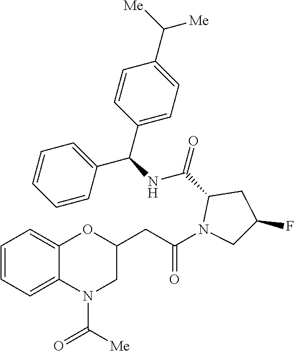 [SMILES: CC(=O)N1CC(CC(=O)N2C[C@H](F)C[C@H]2C(=O)N[C@@H](c2ccccc2)c2ccc(C(C)C)cc2)Oc2ccccc21] | (2S,4R)-1-[2-(4-acetyl-3,4-dihydro- 2H-1,4-benzoxazin-2-yl)acetyl]-4- fluoro-N-[(S)-phenyl[4-(propan-2- yl)phenyl]methyl]pyrrolidine-2- carboxamide |
| GZ-117 | 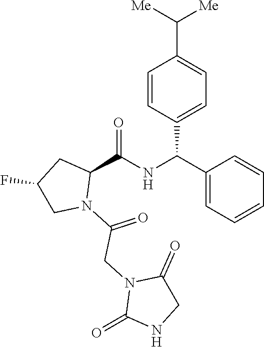 [SMILES: CC(C)c1ccc([C@@H](NC(=O)[C@@H]2C[C@@H](F)CN2C(=O)CN2C(=O)CNC2=O)c2ccccc2)cc1] | (2S,4R)-1-[2-(2,5- dioxoimidazolidin-1-yl)acetyl]-4- fluoro-N-[(S)-phenyl[4-(propan-2- yl)phenyl methyl]pyrrolidine-2- carboxamide |
| GZ-118 | 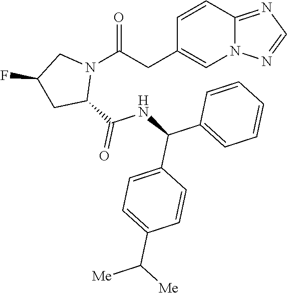 [SMILES: CC(C)c1ccc([C@@H](NC(=O)[C@@H]2C[C@@H](F)CN2C(=O)Cc2ccc3ncnn3c2)c2ccccc2)cc1] | (2S,4R)-4-fluoro-N-[(S)-phenyl[4- (propan-2-yl)phenyl]methyl]-1-(2- {[1,2,4]triazolo[1,5-a]pyridin-6- yl}acetyl)pyrrolidine-2- carboxamide |
| GZ-119 | 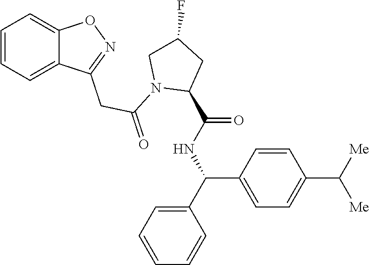 [SMILES: CC(C)c1ccc([C@@H](NC(=O)[C@@H]2C[C@@H](F)CN2C(=O)Cc2noc3ccccc23)c2ccccc2)cc1] | (2S,4R)-1-[2-(1,2-benzoxazol-3- yl)acetyl]-4-fluoro-N-[(S)- phenyl[4-(propan-2- yl)phenyl]methyl]pyrrolidine-2- carboxamide |
| GZ-120 | 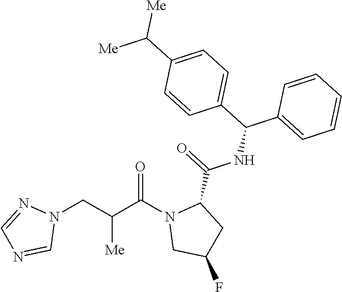 [SMILES: CC(Cn1cncn1)C(=O)N1C[C@H](F)C[C@H]1C(=O)N[C@@H](c1ccccc1)c1ccc(C(C)C)cc1] | (2S,4R)-4-fluoro-1-[2-methyl-3- (1H-1,2,4-triazol-1-yl)propanoyl]- N-[(S)-phenyl[4-(propan-2- yl)phenyl methyl]pyrrolidine-2- carboxamide |
| GZ-121 | 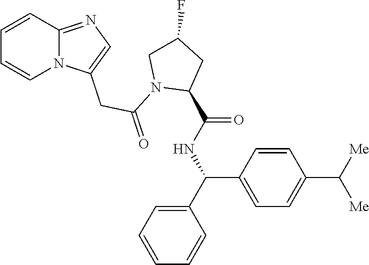 [SMILES: CC(C)c1ccc([C@@H](NC(=O)[C@@H]2C[C@@H](F)CN2C(=O)Cc2cnc3ccccn23)c2ccccc2)cc1] | (2S,4R)-4-fluoro-1-(2- {imidazo[1,2-a]pyridin-3- yl} acetyl)-N-[(S)-phenyl[4- (propan-2- yl)phenyl]methyl]pyrrolidine-2- carboxamide |
| GZ-122 | 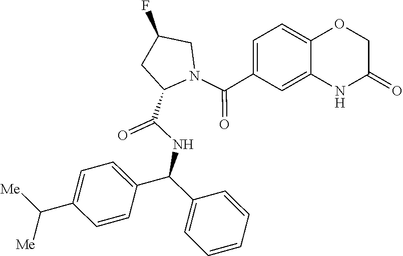 [SMILES: CC(C)c1ccc([C@@H](NC(=O)[C@@H]2C[C@@H](F)CN2C(=O)c2ccc3c(c2)NC(=O)CO3)c2ccccc2)cc1] | (2S,4R)-4-fluoro-1-(3-oxo-3,4- dihydro-2H-1,4-benzoxazine-6- carbonyl)-N-[(S)-phenyl[4-(propan- 2-yl)phenyl methyl]pyrrolidine-2- carboxamide |
| GZ-123 | 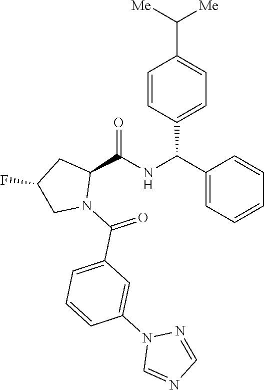 [SMILES: CC(C)c1ccc([C@@H](NC(=O)[C@@H]2C[C@@H](F)CN2C(=O)c2cccc(-n3cncn3)c2)c2ccccc2)cc1] | (2S,4R)-4-fluoro-N-[(S)-phenyl[4- (propan-2-yl)phenyl]methyl]-1-[3- (1H-1,2,4-triazol-1- yl)benzoyl]pyrrolidine-2- carboxamide |
| GZ-124 | 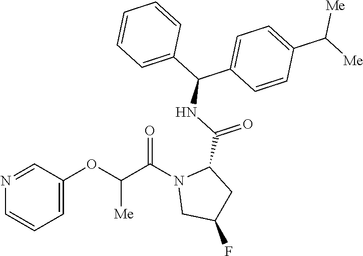 [SMILES: CC(Oc1cccnc1)C(=O)N1C[C@H](F)C[C@H]1C(=O)N[C@@H](c1ccccc1)c1ccc(C(C)C)cc1] | (2S,4R)-4-fluoro-N-[(S)-phenyl[4- (propan-2-yl)phenyl]methyl]-1-[2- (pyridin-3- yloxy)propanoyl]pyrrolidine-2- carboxamide |
| GZ-125 | 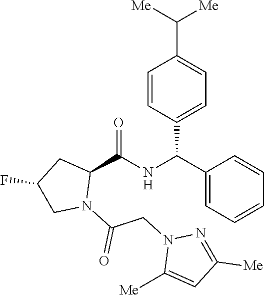 [SMILES: Cc1cc(C)n(CC(=O)N2C[C@H](F)C[C@H]2C(=O)N[C@@H](c2ccccc2)c2ccc(C(C)C)cc2)n1] | (2S,4R)-1-[2-(3,5-dimethyl-1H- pyrazol-1-yl)acetyl]-4-fluoro-N- [(S)-phenyl[4-(propan-2- yl)phenyl methyl]pyrrolidine-2- carboxamide |
| GZ-126 | 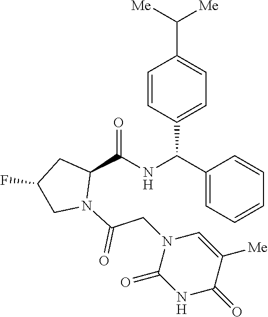 [SMILES: Cc1cn(CC(=O)N2C[C@H](F)C[C@H]2C(=O)N[C@@H](c2ccccc2)c2ccc(C(C)C)cc2)c(=O)[nH]c1=O] | (2S,4R)-4-fluoro-1-[2-(5-methyl- 2,4-dioxo-1,2,3,4- tetrahydropyrimidin-1-yl)acetyl]-N- [(S)-phenyl[4-(propan-2- yl)phenyl methyl]pyrrolidine-2- carboxamide |
| GZ-127 | 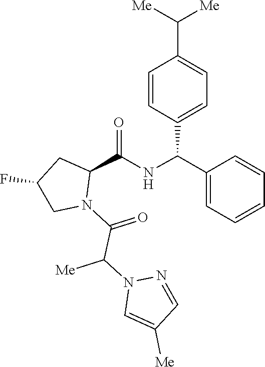 [SMILES: Cc1cnn(C(C)C(=O)N2C[C@H](F)C[C@H]2C(=O)N[C@@H](c2ccccc2)c2ccc(C(C)C)cc2)c1] | (2S,4R)-4-fluoro-1-[2-(4-methyl- 1H-pyrazol-1-yl)propanoyl]-N- [(S)-phenyl[4-(propan-2- yl)phenyl]methyl]pyrrolidine-2- carboxamide |
| GZ-128 | 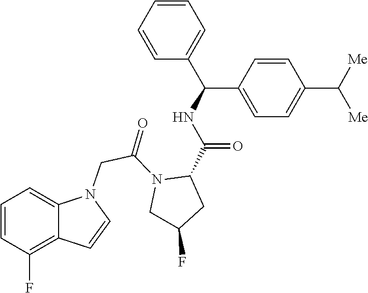 [SMILES: CC(C)c1ccc([C@@H](NC(=O)[C@@H]2C[C@@H](F)CN2C(=O)Cn2ccc3c(F)cccc32)c2ccccc2)cc1] | (2S,4R)-4-fluoro-1-[2-(4-fluoro- 1H-indol-1-yl)acetyl]-N-[(S)- phenyl[4-(propan-2- yl)phenyl]methyl]pyrrolidine-2- carboxamide |
| GZ-129 | 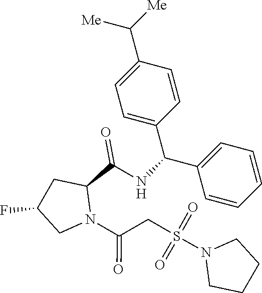 [SMILES: CC(C)c1ccc([C@@H](NC(=O)[C@@H]2C[C@@H](F)CN2C(=O)CS(=O)(=O)N2CCCC2)c2ccccc2)cc1] | (2S,4R)-4-fluoro-N-[(S)-phenyl[4- (propan-2-yl)phenyl]methyl]-1-[2- (pyrrolidine-1- sulfonyl)acetyl]pyrrolidine-2- carboxamide |
| GZ-130 | 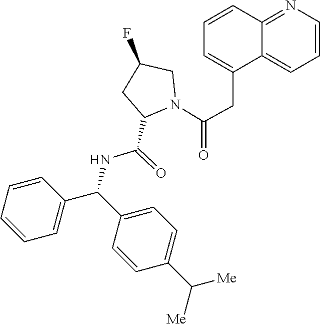 | (2S,4R)-4-fluoro-N-[(S)-phenyl[4- (propan-2-yl)phenyl]methyl]-1-[2- (quinolin-5-yl)acetyl]pyrrolidine-2- carboxamide |
| GZ-131 | 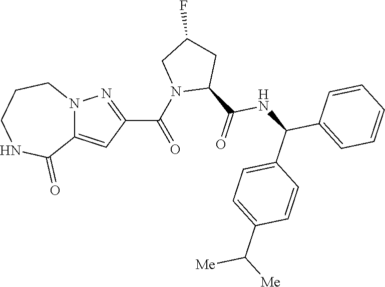 [SMILES: CC(C)c1ccc([C@@H](NC(=O)[C@@H]2C[C@@H](F)CN2C(=O)c2cc3n(n2)CCCNC3=O)c2ccccc2)cc1] | (2S,4R)-4-fluoro-1-{4-oxo- 4H,5H,6H,7H,8H-pyrazolo[1,5- a][1,4]diazepine-2-carbonyl}-N- [(S)-phenyl[4-(propan-2- yl)phenyl]methyl]pyrrolidine-2- carboxamide |
| GZ-132 | 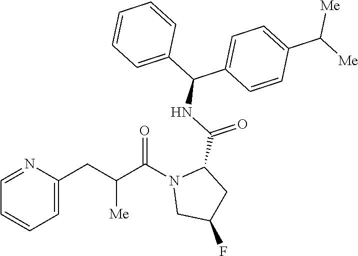 [SMILES: CC(Cc1ccccn1)C(=O)N1C[C@H](F)C[C@H]1C(=O)N[C@@H](c1ccccc1)c1ccc(C(C)C)cc1] | (2S,4R)-4-fluoro-1-[2-methyl-3- (pyridin-2-yl)propanoyl]-N-[(S)- phenyl[4-(propan-2- yl)phenyl\|methyl]pyrrolidine-2- carboxamide |
| GZ-133 | 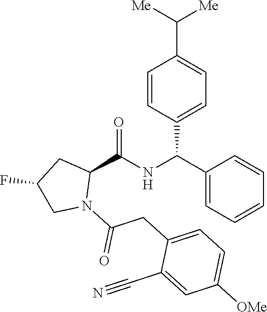 [SMILES: COc1ccc(CC(=O)N2C[C@H](F)C[C@H]2C(=O)N[C@@H](c2ccccc2)c2ccc(C(C)C)cc2)c(C#N)c1] | (2S,4R)-1-[2-(2-cyano-4- methoxyphenyl)acetyl]-4-fluoro-N- [(S)-phenyl[4-(propan-2- yl)phenyl]methyl]pyrrolidine-2- carboxamide |
| GZ-134 | 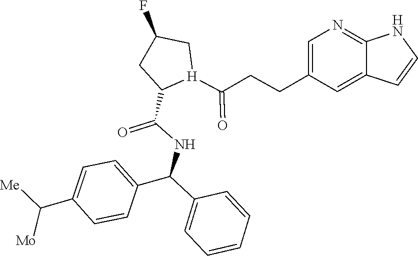 | (2S,4R)-4-fluoro-N-[(S)-phenyl[4- (propan-2-yl)phenyl]methyl]-1-(3- {1H-pyrrolo[2,3-b]pyridin-5- yl } propanoyl)pyrrolidine-2- carboxamide |
| GZ-135 | 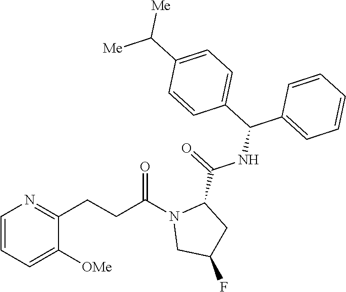 [SMILES: COc1cccnc1CCC(=O)N1C[C@H](F)C[C@H]1C(=O)N[C@@H](c1ccccc1)c1ccc(C(C)C)cc1] | (2S,4R)-4-fluoro-1-[3-(3- methoxypyridin-2-yl)propanoyl]-N- [(S)-phenyl[4-(propan-2- yl)phenyl]methyl]pyrrolidine-2- carboxamide |
| GZ-136 | 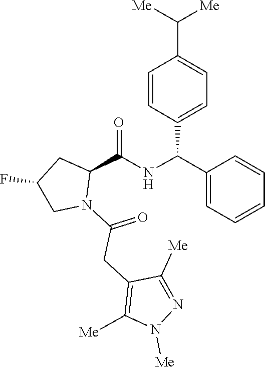 [SMILES: Cc1nn(C)c(C)c1CC(=O)N1C[C@H](F)C[C@H]1C(=O)N[C@@H](c1ccccc1)c1ccc(C(C)C)cc1] | (2S,4R)-4-fluoro-N-[(S)-phenyl[4- (propan-2-yl)phenyl]methyl]-1-[2- (1,3,5-trimethyl-1H-pyrazol-4- yl)acetyl]pyrrolidine-2- carboxamide |
| GZ-137 | 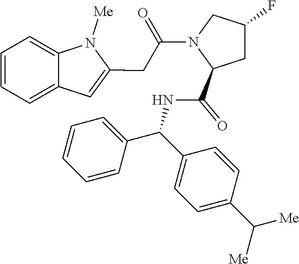 [SMILES: CC(C)c1ccc([C@@H](NC(=O)[C@@H]2C[C@@H](F)CN2C(=O)Cc2cc3ccccc3n2C)c2ccccc2)cc1] | (2S,4R)-4-fluoro-1-[2-(1-methyl- 1H-indol-2-yl)acetyl]-N-[(S)- phenyl[4-(propan-2- yl)phenyl\|methyl]pyrrolidine-2- carboxamide |
| GZ-138 | 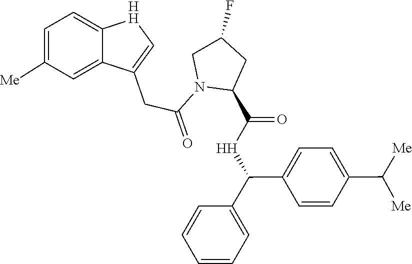 | (2S,4R)-4-fluoro-1-[2-(5-methyl- 1H-indol-3-yl)acetyl]-N-[(S)- phenyl[4-(propan-2- yl)phenyl\|methyl]pyrrolidine-2- carboxamide |
| GZ-139 | 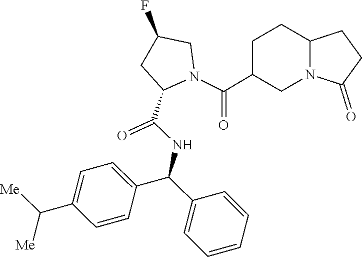 [SMILES: CC(C)c1ccc([C@@H](NC(=O)[C@@H]2C[C@@H](F)CN2C(=O)C2CCC3CCC(=O)N3C2)c2ccccc2)cc1] | (2S,4R)-4-fluoro-1-(3-oxo- octahydroindolizine-6-carbonyl)-N- [(S)-phenyl[4-(propan-2- yl)phenyl]methyl]pyrrolidine-2- carboxamide |
| GZ-140 | 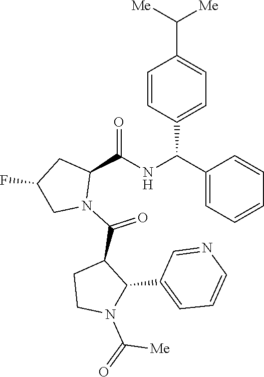 [SMILES: CC(=O)N1CC[C@@H](C(=O)N2C[C@H](F)C[C@H]2C(=O)N[C@@H](c2ccccc2)c2ccc(C(C)C)cc2)[C@@H]1c1cccnc1] | (2S,4R)-1-[(2R,3R)-1-acetyl-2- (pyridin-3-yl)pyrrolidine-3- carbonyl]-4-fluoro-N-[(S)- phenyl[4-(propan-2- yl)phenyl]methyl]pyrrolidine-2- carboxamide |
| GZ-141 | 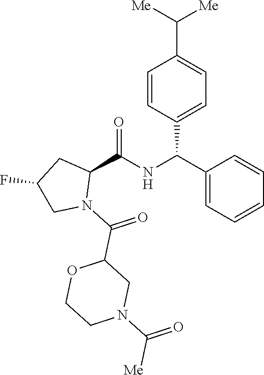 [SMILES: CC(=O)N1CCOC(C(=O)N2C[C@H](F)C[C@H]2C(=O)N[C@@H](c2ccccc2)c2ccc(C(C)C)cc2)C1] | (2S,4R)-1-(4-acetylmorpholine-2- carbonyl)-4-fluoro-N-[(S)- phenyl[4-(propan-2- yl)phenyl methyl]pyrrolidine-2- carboxamide |
| GZ-142 | 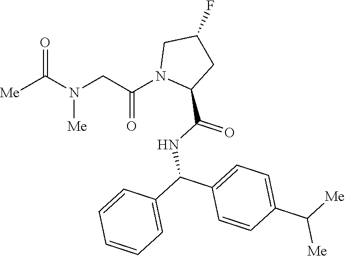 [SMILES: CC(=O)N(C)CC(=O)N1C[C@H](F)C[C@H]1C(=O)N[C@@H](c1ccccc1)c1ccc(C(C)C)cc1] | (2S,4R)-4-fluoro-1-[2-(N- methylacetamido)acetyl]-N-[(S)- phenyl[4-(propan-2- yl)phenyl]methyl]pyrrolidine-2- carboxamide |
| GZ-143 | 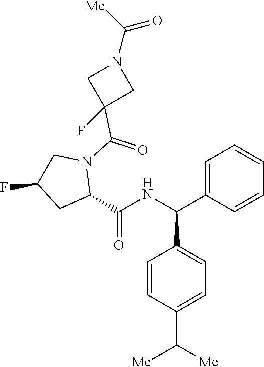 [SMILES: CC(=O)N1CC(F)(C(=O)N2C[C@H](F)C[C@H]2C(=O)N[C@@H](c2ccccc2)c2ccc(C(C)C)cc2)C1] | (2S,4R)-1-(1-acetyl-3- fluoroazetidine-3-carbonyl)-4- fluoro-N-[(S)-phenyl[4-(propan-2- yl)phenyl]methyl]pyrrolidine-2- carboxamide |
| GZ-144 | 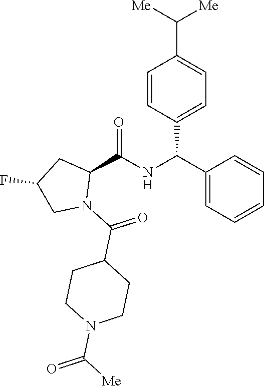 [SMILES: CC(=O)N1CCC(C(=O)N2C[C@H](F)C[C@H]2C(=O)N[C@@H](c2ccccc2)c2ccc(C(C)C)cc2)CC1] | (2S,4R)-1-(1-acetylpiperidine-4- carbonyl)-4-fluoro-N-[(S)- phenyl[4-(propan-2- yl)phenyl]methyl]pyrrolidine-2- carboxamide |
| GZ-145 | 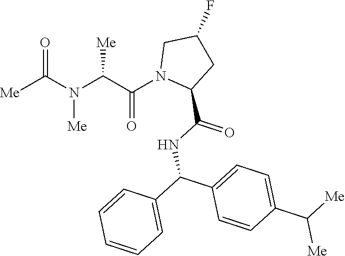 [SMILES: CC(=O)N(C)[C@H](C)C(=O)N1C[C@H](F)C[C@H]1C(=O)N[C@@H](c1ccccc1)c1ccc(C(C)C)cc1] | (2S,4R)-4-fluoro-1-[(2R)-2-(N- methylacetamido)propanoyl]-N- [(S)-phenyl[4-(propan-2- yl)phenyl]methyl]pyrrolidine-2- carboxamide |
| GZ-146 | 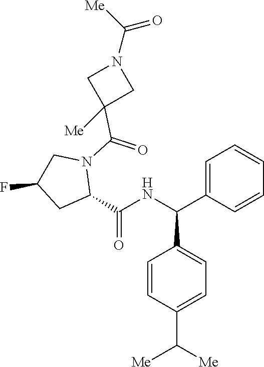 [SMILES: CC(=O)N1CC(C)(C(=O)N2C[C@H](F)C[C@H]2C(=O)N[C@@H](c2ccccc2)c2ccc(C(C)C)cc2)C1] | (2S,4R)-1-(1-acetyl-3- methylazetidine-3-carbonyl)-4- fluoro-N-[(S)-phenyl[4-(propan-2- yl)phenyl\|methyl]pyrrolidine-2- carboxamide |
| GZ-147 | 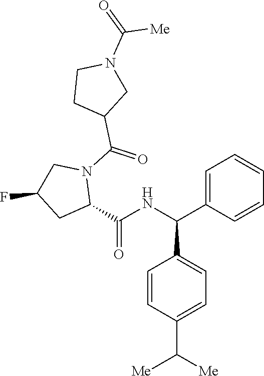 [SMILES: CC(=O)N1CCC(C(=O)N2C[C@H](F)C[C@H]2C(=O)N[C@@H](c2ccccc2)c2ccc(C(C)C)cc2)C1] | (2S,4R)-1-(1-acetylpyrrolidine-3- carbonyl)-4-fluoro-N-[(S)- phenyl[4-(propan-2- yl)phenyl methyl]pyrrolidine-2- carboxamide |
| GZ-148 | 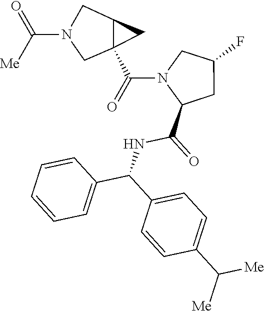 [SMILES: CC(=O)N1C[C@H]2C[C@@]2(C(=O)N2C[C@H](F)C[C@H]2C(=O)N[C@@H](c2ccccc2)c2ccc(C(C)C)cc2)C1] | (2S,4R)-1-[(1S,5S)-3-acetyl-3- azabicyclo[3.1.0]hexane-1- carbonyl]-4-fluoro-N-[(S)- phenyl[4-(propan-2- yl)phenyl methyl]pyrrolidine-2- carboxamide |
| GZ-149 | 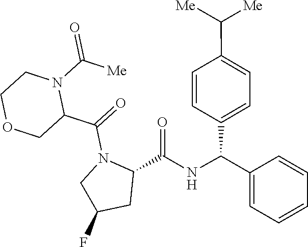 [SMILES: CC(=O)N1CCOCC1C(=O)N1C[C@H](F)C[C@H]1C(=O)N[C@@H](c1ccccc1)c1ccc(C(C)C)cc1] | (2S,4R)-1-(4-acetylmorpholine-3- carbonyl)-4-fluoro-N-[(S)- phenyl[4-(propan-2- yl)phenyl methyl]pyrrolidine-2- carboxamide |
| GZ-150 | 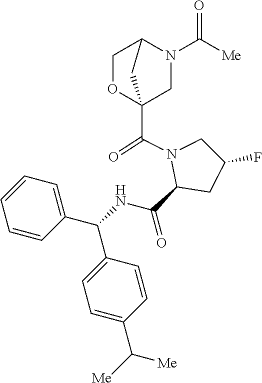 [SMILES: CC(=O)N1C[C@]2(C(=O)N3C[C@H](F)C[C@H]3C(=O)N[C@@H](c3ccccc3)c3ccc(C(C)C)cc3)CC1CO2] | (2S,4R)-1-[(1S)-5-acetyl-2-oxa-5- azabicyclo[2.2.1 ]heptane-1- carbonyl]-4-fluoro-N-[(S)- phenyl[4-(propan-2- yl)phenyl]methyl]pyrrolidine-2- carboxamide |
| GZ-151 | 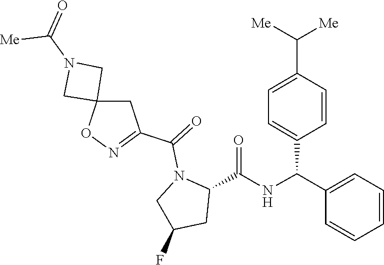 [SMILES: CC(=O)N1CC2(CC(C(=O)N3C[C@H](F)C[C@H]3C(=O)N[C@@H](c3ccccc3)c3ccc(C(C)C)cc3)=NO2)C1] | (2S,4R)-1-{2-acetyl-5-oxa-2,6- diazaspiro[3.4]oct-6-ene-7- carbonyl}-4-fluoro-N-[(S)- phenyl[4-(propan-2- yl)phenyl]methyl]pyrrolidine-2- carboxamide |
| GZ-152 | 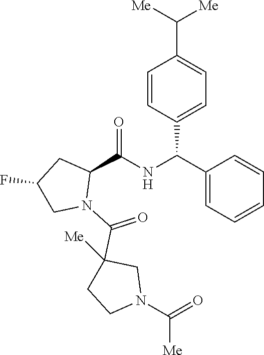 [SMILES: CC(=O)N1CCC(C)(C(=O)N2C[C@H](F)C[C@H]2C(=O)N[C@@H](c2ccccc2)c2ccc(C(C)C)cc2)C1] | (2S,4R)-1-(1-acetyl-3- methylpyrrolidine-3-carbonyl)-4- fluoro-N-[(S)-phenyl[4-(propan-2- yl)phenyl]methyl]pyrrolidine-2- carboxamide |
| GZ-153 | 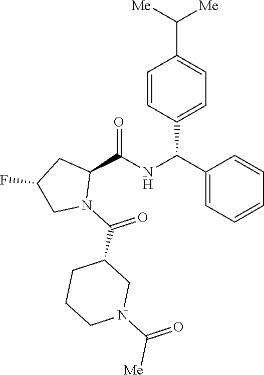 [SMILES: CC(=O)N1CCC[C@H](C(=O)N2C[C@H](F)C[C@H]2C(=O)N[C@@H](c2ccccc2)c2ccc(C(C)C)cc2)C1] | (2S,4R)-1-[(3S)-1-acetylpiperidine- 3-carbonyl]-4-fluoro-N-[(S)- phenyl[4-(propan-2- yl)phenyl]methyl]pyrrolidine-2- carboxamide |
| GZ-154 | 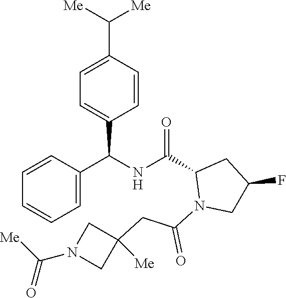 [SMILES: CC(=O)N1CC(C)(CC(=O)N2C[C@H](F)C[C@H]2C(=O)N[C@@H](c2ccccc2)c2ccc(C(C)C)cc2)C1] | (2S,4R)-1-[2-(1-acetyl-3- methylazetidin-3-yl)acetyl]-4- fluoro-N-[(S)-phenyl[4-(propan-2- yl)phenyl]methyl]pyrrolidine-2- carboxamide |
| GZ-155 | 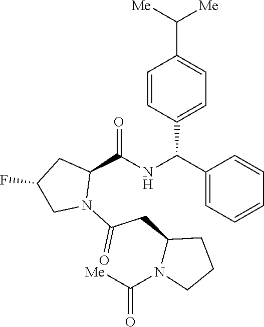 [SMILES: CC(=O)N1CCC[C@@H]1CC(=O)N1C[C@H](F)C[C@H]1C(=O)N[C@@H](c1ccccc1)c1ccc(C(C)C)cc1] | (2S,4R)-1-{2-[(2R)-1- acetylpyrrolidin-2-yl]acetyl}-4- fluoro-N-[(S)-phenyl[4-(propan-2- yl)phenyl]methyl]pyrrolidine-2- carboxamide |
| GZ-156 | 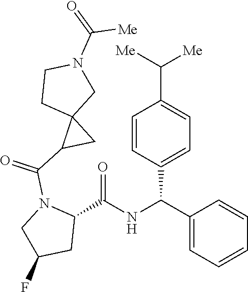 [SMILES: CC(=O)N1CCC2(CC2C(=O)N2C[C@H](F)C[C@H]2C(=O)N[C@@H](c2ccccc2)c2ccc(C(C)C)cc2)C1] | (2S,4R)-1-{5-acetyl-5- azaspiro[2.4]heptane-1-carbonyl}- 4-fluoro-N-[(S)-phenyl[4-(propan- 2-yl)phenyl]methyl]pyrrolidine-2- carboxamide |
| GZ-157 | 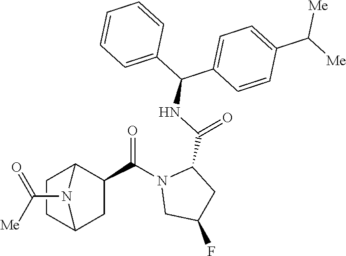 [SMILES: CC(=O)N1C2CCC1[C@@H](C(=O)N1C[C@H](F)C[C@H]1C(=O)N[C@@H](c1ccccc1)c1ccc(C(C)C)cc1)C2] | (2S,4R)-1-[(2S)-7-acetyl-7- azabicyclo[2.2.1 ]heptane-2- carbonyl]-4-fluoro-N-[(S)- phenyl[4-(propan-2- yl)phenyl]methyl]pyrrolidine-2- carboxamide |
| GZ-158 | 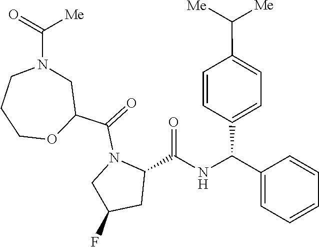 [SMILES: CC(=O)N1CCCOC(C(=O)N2C[C@H](F)C[C@H]2C(=O)N[C@@H](c2ccccc2)c2ccc(C(C)C)cc2)C1] | (2S,4R)-1-[(2R)-4-acetyl-1,4- oxazepane-2-carbonyl]-4-fluoro-N- [(S)-phenyl[4-(propan-2- yl)phenyl methyl]pyrrolidine-2- carboxamide |
| GZ-159 | 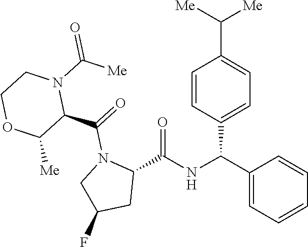 [SMILES: CC(=O)N1CCO[C@@H](C)[C@@H]1C(=O)N1C[C@H](F)C[C@H]1C(=O)N[C@@H](c1ccccc1)c1ccc(C(C)C)cc1] | (2S,4R)-1-[(2S,3R)-4-acetyl-2- methylmorpholine-3-carbonyl]-4- fluoro-N-[(S)-phenyl[4-(propan-2- yl)phenylmethyl]pyrrolidine-2- carboxamide |
| GZ-160 | 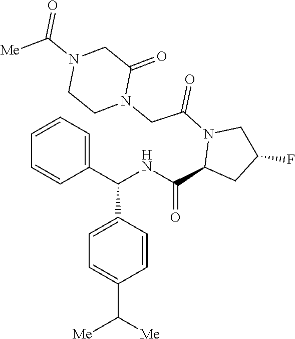 [SMILES: CC(=O)N1CCN(CC(=O)N2C[C@H](F)C[C@H]2C(=O)N[C@@H](c2ccccc2)c2ccc(C(C)C)cc2)C(=O)C1] | (2S,4R)-1-[2-(4-acetyl-2- oxopiperazin-1-yl)acetyl]-4-fluoro- N-[(S)-phenyl[4-(propan-2- yl)phenyl]methyl]pyrrolidine-2- carboxamide |
| GZ-161 | 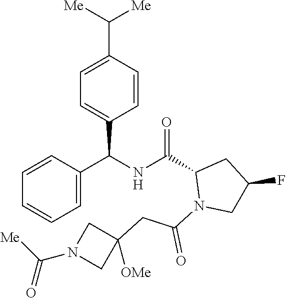 [SMILES: COC1(CC(=O)N2C[C@H](F)C[C@H]2C(=O)N[C@@H](c2ccccc2)c2ccc(C(C)C)cc2)CN(C(C)=O)C1] | (2S,4R)-1-[2-(1-acetyl-3- methoxyazetidin-3-yl)acetyl]-4- fluoro-N-[(S)-phenyl[4-(propan-2- yl)phenyl]methyl]pyrrolidine-2- carboxamide |
| GZ-162 | 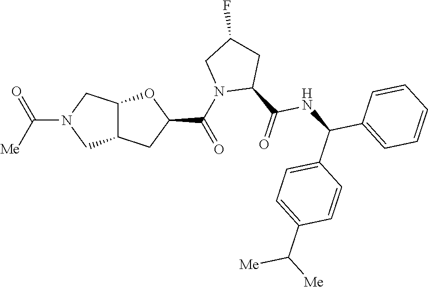 [SMILES: CC(=O)N1C[C@H]2C[C@H](C(=O)N3C[C@H](F)C[C@H]3C(=O)N[C@@H](c3ccccc3)c3ccc(C(C)C)cc3)O[C@H]2C1] | (2S,4R)-1-[(2R,3aR,6aR)-5-acetyl- hexahydro-2H-furo[2,3-c]pyrrole- 2-carbonyl]-4-fluoro-N-[(S)- phenyl[4-(propan-2- yl)phenyl methyl]pyrrolidine-2- carboxamide |
| GZ-163 | 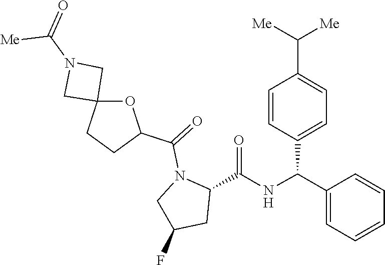 [SMILES: CC(=O)N1CC2(CCC(C(=O)N3C[C@H](F)C[C@H]3C(=O)N[C@@H](c3ccccc3)c3ccc(C(C)C)cc3)O2)C1] | (2S,4R)-1-{2-acetyl-5-oxa-2- azaspiro[3.4]octane-6-carbonyl}-4- fluoro-N-[(S)-phenyl[4-(propan-2- yl)phenyl\|methyl]pyrrolidine-2- carboxamide |
| GZ-164 | 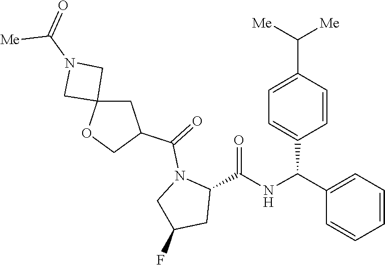 [SMILES: CC(=O)N1CC2(CC(C(=O)N3C[C@H](F)C[C@H]3C(=O)N[C@@H](c3ccccc3)c3ccc(C(C)C)cc3)CO2)C1] | (2S,4R)-1-{2-acetyl-5-oxa-2- azaspiro[3.4]octane-7-carbonyl}-4- fluoro-N-[(S)-phenyl[4-(propan-2- yl)phenyl]methyl]pyrrolidine-2- carboxamide |
| GZ-165 | 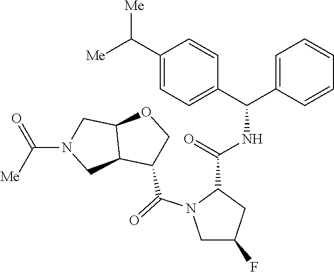 | (2S,4R)-1-[(3R,3aS,6aS)-5-acetyl- hexahydro-2H-furo[2,3-c]pyrrole- 3-carbonyl]-4-fluoro-N-[(S)- phenyl[4-(propan-2- yl)phenyl]methyl]pyrrolidine-2- carboxamide |
| GZ-166 | 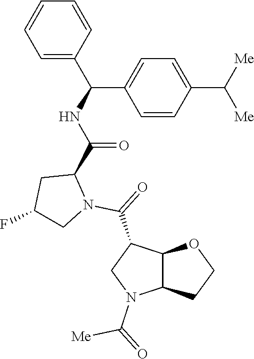 [SMILES: CC(=O)N1C[C@H](C(=O)N2C[C@H](F)C[C@H]2C(=O)N[C@@H](c2ccccc2)c2ccc(C(C)C)cc2)[C@H]2OCC[C@H]21] | (2S,4R)-1-[(3aR,6S,6aR)-4-acetyl- hexahydro-2H-furo[3,2-b]pyrrole- 6-carbonyl]-4-fluoro-N-[(S)- phenyl[4-(propan-2- yl)phenyl methyl]pyrrolidine-2- carboxamide |
| GZ-167 | 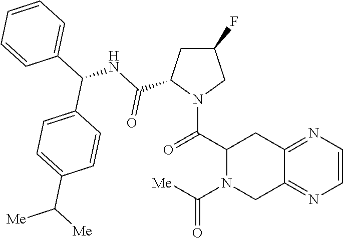 [SMILES: CC(=O)N1Cc2nccnc2CC1C(=O)N1C[C@H](F)C[C@H]1C(=O)N[C@@H](c1ccccc1)c1ccc(C(C)C)cc1] | (2S,4R)-1-{6-acetyl-5H,6H,7H,8H- pyrido[3,4-b]pyrazine-7-carbonyl}- 4-fluoro-N-[(S)-phenyl[4-(propan- 2-yl)phenyl methyl]pyrrolidine-2- carboxamide |
| GZ-168 | 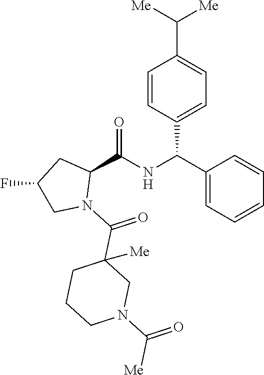 [SMILES: CC(=O)N1CCCC(C)(C(=O)N2C[C@H](F)C[C@H]2C(=O)N[C@@H](c2ccccc2)c2ccc(C(C)C)cc2)C1] | (2S,4R)-1-(1-acetyl-3- methylpiperidine-3-carbonyl)-4- fluoro-N-[(S)-phenyl[4-(propan-2- yl)phenyl]methyl]pyrrolidine-2- carboxamide |
| GZ-169 | 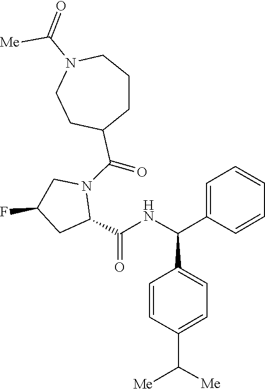 [SMILES: CC(=O)N1CCCC(C(=O)N2C[C@H](F)C[C@H]2C(=O)N[C@@H](c2ccccc2)c2ccc(C(C)C)cc2)CC1] | (2S,4R)-1-(1-acetylazepane-4- carbonyl)-4-fluoro-N-[(S)- phenyl[4-(propan-2- yl)phenyl]methyl]pyrrolidine-2- carboxamide |
| GZ-170 | 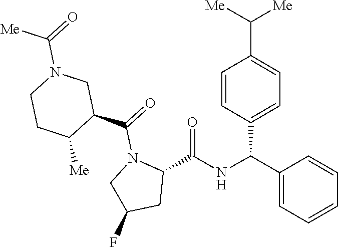 [SMILES: CC(=O)N1CC[C@@H](C)[C@H](C(=O)N2C[C@H](F)C[C@H]2C(=O)N[C@@H](c2ccccc2)c2ccc(C(C)C)cc2)C1] | (2S,4R)-1-[(3S,4R)-1-acetyl-4- methylpiperidine-3-carbonyl]-4- fluoro-N-[(S)-phenyl[4-(propan-2- yl)phenyl\|methyl]pyrrolidine-2- carboxamide |
| GZ-171 | 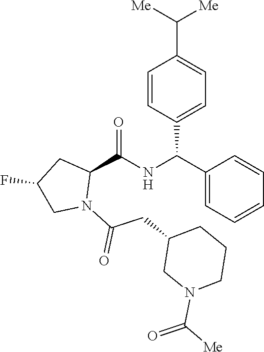 [SMILES: CC(=O)N1CCC[C@@H](CC(=O)N2C[C@H](F)C[C@H]2C(=O)N[C@@H](c2ccccc2)c2ccc(C(C)C)cc2)C1] | (2S,4R)-1-{2-[(3S)-1- acetylpiperidin-3-yl]acetyl}-4- fluoro-N-[(S)-phenyl[4-(propan-2- yl)phenyl]methyl]pyrrolidine-2- carboxamide |
| GZ-172 | 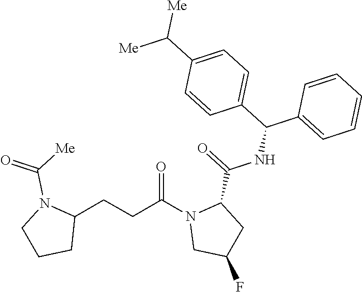 [SMILES: CC(=O)N1CCCC1CCC(=O)N1C[C@H](F)C[C@H]1C(=O)N[C@@H](c1ccccc1)c1ccc(C(C)C)cc1] | (2S,4R)-1-[3-(1-acetylpyrrolidin-2- yl)propanoyl]-4-fluoro-N-[(S)- phenyl[4-(propan-2- yl)phenyl methyl]pyrrolidine-2- carboxamide |
| GZ-173 | 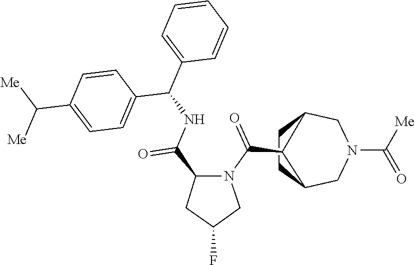 [SMILES: CC(=O)N1C[C@H]2CC[C@@H](C1)[C@@H]2C(=O)N1C[C@H](F)C[C@H]1C(=O)N[C@@H](c1ccccc1)c1ccc(C(C)C)cc1] | (2S,4R)-1-[(1R,5S,8S)-3-acetyl-3- azabicyclo[3.2.1]octane-8- carbonyl]-4-fluoro-N-[(S)- phenyl[4-(propan-2- yl)phenyl]methyl]pyrrolidine-2- carboxamide |
| GZ-174 | 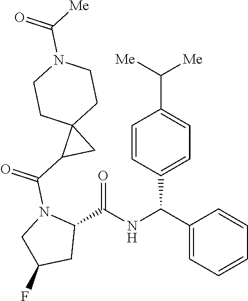 [SMILES: CC(=O)N1CCC2(CC1)CC2C(=O)N1C[C@H](F)C[C@H]1C(=O)N[C@@H](c1ccccc1)c1ccc(C(C)C)cc1] | (2S,4R)-1-{6-acetyl-6- azaspiro[2.5]octane-1-carbonyl}-4- fluoro-N-[(S)-phenyl[4-(propan-2- yl)phenyl]methyl]pyrrolidine-2- carboxamide |
| GZ-175 | 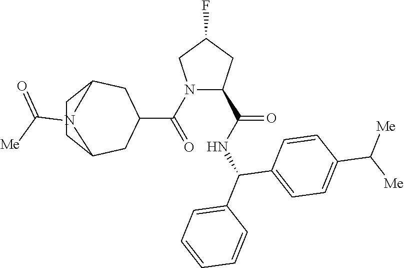 [SMILES: CC(=O)N1C2CCC1CC(C(=O)N1C[C@H](F)C[C@H]1C(=O)N[C@@H](c1ccccc1)c1ccc(C(C)C)cc1)C2] | (2S,4R)-1-{8-acetyl-8- azabicyclo[3.2.1 ]octane-3- carbonyl}-4-fluoro-N-[(S)- phenyl[4-(propan-2- yl)phenyl methyl]pyrrolidine-2- carboxamide |
| GZ-176 | 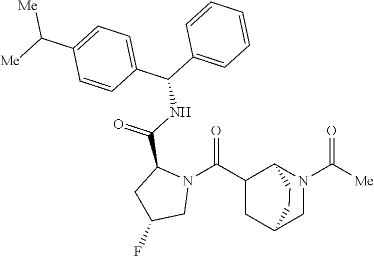 [SMILES: CC(=O)N1C[C@@H]2CC[C@H]1C(C(=O)N1C[C@H](F)C[C@H]1C(=O)N[C@@H](c1ccccc1)c1ccc(C(C)C)cc1)C2] | (2S,4R)-1-[(1S,4R)-2-acetyl-2- azabicyclo[2.2.2]octane-6- carbonyl]-4-fluoro-N-[(S)- phenyl[4-(propan-2- yl)phenyl]methyl]pyrrolidine-2- carboxamide |
| GZ-177 | 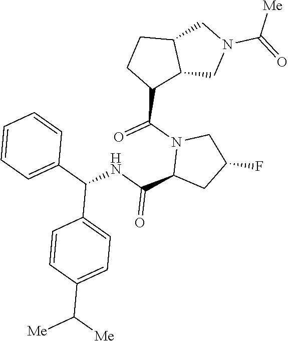 | (2S,4R)-1-[(3aS,4S,6aS)-2-acetyl- octahydrocyclopenta[c]pyrrole-4- carbonyl]-4-fluoro-N-[(S)- phenyl[4-(propan-2- yl)phenyl]methyl]pyrrolidine-2- carboxamide |
| GZ-178 | 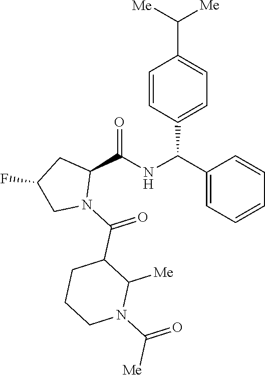 [SMILES: CC(=O)N1CCCC(C(=O)N2C[C@H](F)C[C@H]2C(=O)N[C@@H](c2ccccc2)c2ccc(C(C)C)cc2)C1C] | (2S,4R)-1-(1-acetyl-2- methylpiperidine-3-carbonyl)-4- fluoro-N-[(S)-phenyl[4-(propan-2- yl)phenyl]methyl]pyrrolidine-2- carboxamide |
| GZ-179 | 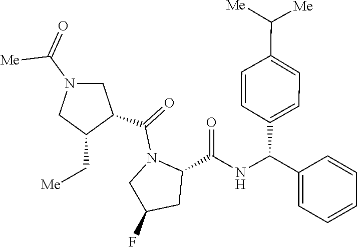 [SMILES: CC[C@@H]1CN(C(C)=O)C[C@@H]1C(=O)N1C[C@H](F)C[C@H]1C(=O)N[C@@H](c1ccccc1)c1ccc(C(C)C)cc1] | (2S,4R)-1-[(3R,4S)-1-acetyl-4- ethylpyrrolidine-3-carbonyl]-4- fluoro-N-[(S)-phenyl[4-(propan-2- yl)phenyl\|methyl]pyrrolidine-2- carboxamide |
| GZ-180 | 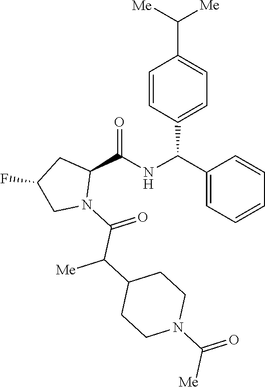 [SMILES: CC(=O)N1CCC(C(C)C(=O)N2C[C@H](F)C[C@H]2C(=O)N[C@@H](c2ccccc2)c2ccc(C(C)C)cc2)CC1] | (2S,4R)-1-[2-(1-acetylpiperidin-4- yl)propanoyl]-4-fluoro-N-[(S)- phenyl[4-(propan-2- yl)phenyl]methyl]pyrrolidine-2- carboxamide |
| GZ-181 | 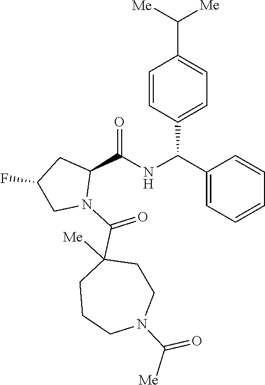 [SMILES: CC(=O)N1CCCC(C)(C(=O)N2C[C@H](F)C[C@H]2C(=O)N[C@@H](c2ccccc2)c2ccc(C(C)C)cc2)CC1] | (2S,4R)-1-(1-acetyl-4- methylazepane-4-carbonyl)-4- fluoro-N-[(S)-phenyl[4-(propan-2- yl)phenyl]methyl]pyrrolidine-2- carboxamide |
| GZ-182 | 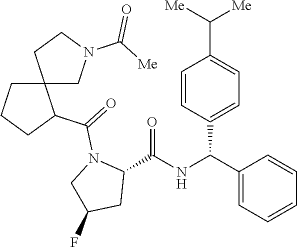 [SMILES: CC(=O)N1CCC2(CCCC2C(=O)N2C[C@H](F)C[C@H]2C(=O)N[C@@H](c2ccccc2)c2ccc(C(C)C)cc2)C1] | (2S,4R)-1-{2-acetyl-2- azaspiro[4.4]nonane-6-carbonyl} -4- fluoro-N-[(S)-phenyl[4-(propan-2- yl)phenyl]methyl]pyrrolidine-2- carboxamide |
| GZ-183 | 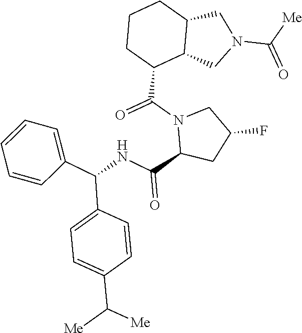 [SMILES: CC(=O)N1C[C@H]2CCC[C@@H](C(=O)N3C[C@H](F)C[C@H]3C(=O)N[C@@H](c3ccccc3)c3ccc(C(C)C)cc3)[C@H]2C1] | (2S,4R)-1-[(3aS,4R,7aS)-2-acetyl- octahydro-1H-isoindole-4- carbonyl]-4-fluoro-N-[(S)- phenyl[4-(propan-2- yl)phenyl\|methyl]pyrrolidine-2- carboxamide |
| GZ-184 | 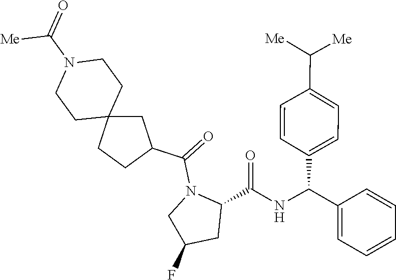 [SMILES: CC(=O)N1CCC2(CCC(C(=O)N3C[C@H](F)C[C@H]3C(=O)N[C@@H](c3ccccc3)c3ccc(C(C)C)cc3)C2)CC1] | (2S,4R)-1-{8-acetyl-8- azaspiro[4.5]decane-2-carbonyl}-4- fluoro-N-[(S)-phenyl[4-(propan-2- yl)phenyl]methyl]pyrrolidine-2- carboxamide |
| GZ-185 | 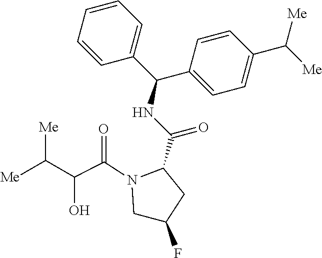 [SMILES: CC(C)c1ccc([C@@H](NC(=O)[C@@H]2C[C@@H](F)CN2C(=O)C(O)C(C)C)c2ccccc2)cc1] | (2S,4R)-4-fluoro-1-(2-hydroxy-3- methylbutanoyl)-N-[(S)-phenyl[4- (propan-2- yl)phenyl methyl]pyrrolidine-2- carboxamide |
| GZ-186 | 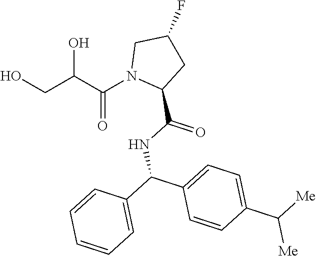 [SMILES: CC(C)c1ccc([C@@H](NC(=O)[C@@H]2C[C@@H](F)CN2C(=O)C(O)CO)c2ccccc2)cc1] | (2S,4R)-1-(2,3- dihydroxypropanoyl)-4-fluoro-N- [(S)-phenyl[4-(propan-2- yl)phenyl]methyl]pyrrolidine-2- carboxamide |
| GZ-187 | 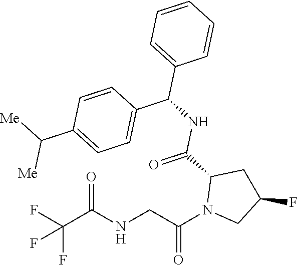 [SMILES: CC(C)c1ccc([C@@H](NC(=O)[C@@H]2C[C@@H](F)CN2C(=O)CNC(=O)C(F)(F)F)c2ccccc2)cc1] | (2S,4R)-4-fluoro-N-[(S)-phenyl[4- (propan-2-yl)phenyl]methyl]-1-[2- (2,2,2- trifluoroacetamido)acetyl]pyrrolidine- 2-carboxamide |
| GZ-188 | 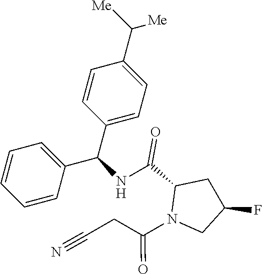 [SMILES: CC(C)c1ccc([C@@H](NC(=O)[C@@H]2C[C@@H](F)CN2C(=O)CC#N)c2ccccc2)cc1] | (2S,4R)-1-(2-cyanoacetyl)-4- fluoro-N-[(S)-phenyl[4-(propan-2- yl)phenyl methyl]pyrrolidine-2- carboxamide |
| GZ-189 | 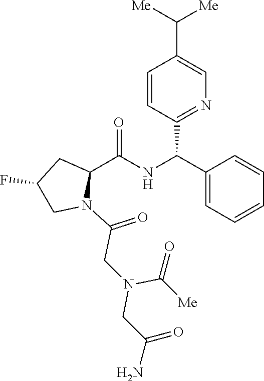 [SMILES: CC(=O)N(CC(N)=O)CC(=O)N1C[C@H](F)C[C@H]1C(=O)N[C@@H](c1ccccc1)c1ccc(C(C)C)cn1] | (2S,4R)-1-{2-[N- (carbamoylmethyl)acetamido]acetyl} -4-fluoro-N-[(S)-phenyl[4- (propan-2- yl)phenyl]methyl]pyrrolidine-2- carboxamide |
| GZ-190 | 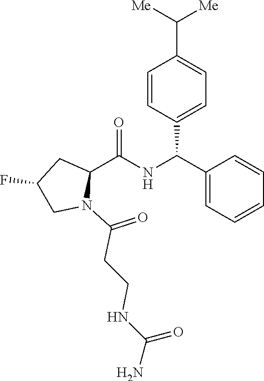 [SMILES: CC(C)c1ccc([C@@H](NC(=O)[C@@H]2C[C@@H](F)CN2C(=O)CCNC(N)=O)c2ccccc2)cc1] | (2S,4R)-1-(3-acetamidopropanoyl)- 4-fluoro-N-[(S)-phenyl[4-(propan- 2-yl)phenyl]methyl]pyrrolidine-2- carboxamide |
| GZ-191 | 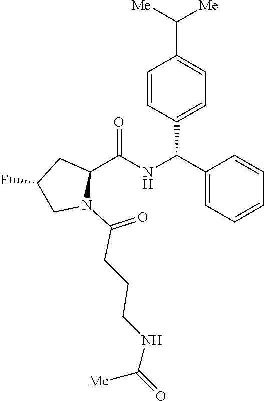 [SMILES: CC(=O)NCCCC(=O)N1C[C@H](F)C[C@H]1C(=O)N[C@@H](c1ccccc1)c1ccc(C(C)C)cc1] | (2S,4R)-1-(4-acetamidobutanoyl)- 4-fluoro-N-[(S)-phenyl[4-(propan- 2-yl)phenyl]methyl]pyrrolidine-2- carboxamide |
| GZ-192 | 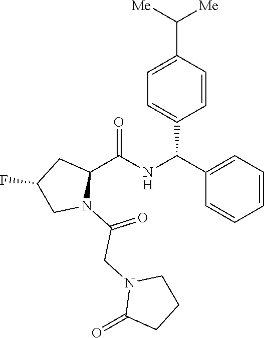 [SMILES: CC(C)c1ccc([C@@H](NC(=O)[C@@H]2C[C@@H](F)CN2C(=O)CN2CCCC2=O)c2ccccc2)cc1] | (2S,4R)-4-fluoro-1-[2-(2- oxopyrrolidin-1-yl)acetyl]-N-[(S)- phenyl[4-(propan-2- yl)phenyl\|methyl]pyrrolidine-2- carboxamide |
| GZ-193 | 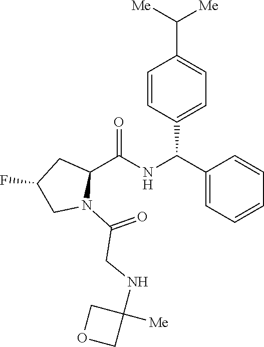 [SMILES: CC(C)c1ccc([C@@H](NC(=O)[C@@H]2C[C@@H](F)CN2C(=O)CNC2(C)COC2)c2ccccc2)cc1] | (2S,4R)-4-fluoro-1-{2-[(3- methyloxetan-3-yl)amino]acetyl}- N-[(S)-phenyl[4-(propan-2- yl)phenyl]methyl]pyrrolidine-2- carboxamide |
| GZ-194 | 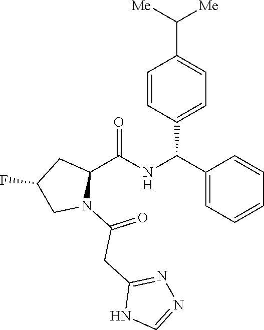 [SMILES: CC(C)c1ccc([C@@H](NC(=O)[C@@H]2C[C@@H](F)CN2C(=O)Cc2nnc[nH]2)c2ccccc2)cc1] | (2S,4R)-4-fluoro-N-[(S)-phenyl[4- (propan-2-yl)phenyl]methyl]-1-[2- (4H-1,2,4-triazol-3- yl)acetyl]pyrrolidine-2- carboxamide |
| GZ-195 | 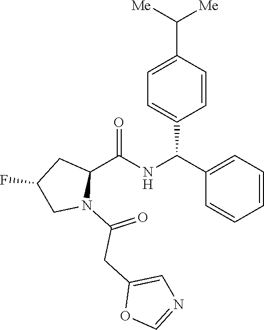 [SMILES: CC(C)c1ccc([C@@H](NC(=O)[C@@H]2C[C@@H](F)CN2C(=O)Cc2cnco2)c2ccccc2)cc1] | (2S,4R)-4-fluoro-1-[2-(1,3-oxazol- 5-yl)acetyl]-N-[(S)-phenyl[4- (propan-2- yl)phenyl]methyl]pyrrolidine-2- carboxamide |
| GZ-196 | 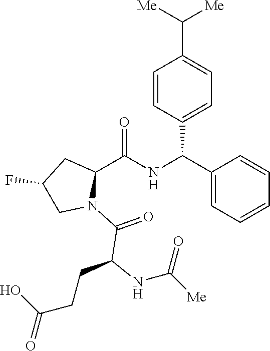 [SMILES: CC(=O)N[C@@H](CCC(=O)O)C(=O)N1C[C@H](F)C[C@H]1C(=O)N[C@@H](c1ccccc1)c1ccc(C(C)C)cc1] | (4S)-4-acetamido-5-[(2S,4R)-4- fluoro-2-{[(S)-phenyl[4-(propan-2- yl)phenyl]methyl]carbamoyl} pyrrolidin-1-yl]-5-oxopentanoic acid |
| GZ-197 | 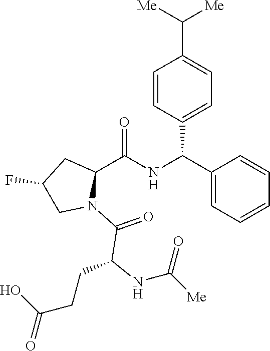 [SMILES: CC(=O)N[C@H](CCC(=O)O)C(=O)N1C[C@H](F)C[C@H]1C(=O)N[C@@H](c1ccccc1)c1ccc(C(C)C)cc1] | (4R)-4-acetamido-5-[(2S,4R)-4- fluoro-2-{[(S)-phenyl[4-(propan-2- yl)phenyl]methyl]carbamoyl} pyrrolidin-1-yl]-5-oxopentanoic acid |
| GZ-198 | 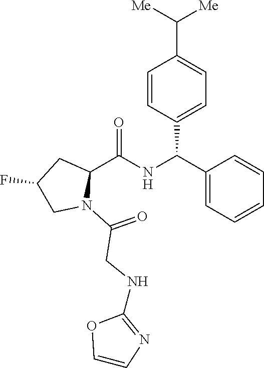 [SMILES: CC(C)c1ccc([C@@H](NC(=O)[C@@H]2C[C@@H](F)CN2C(=O)CNc2ncco2)c2ccccc2)cc1] | (2S,4R)-4-fluoro-1-{2-[(1,3- oxazol-2-yl)amino]acetyl}-N-[(S)- phenyl[4-(propan-2- yl)phenyl\|methyl]pyrrolidine-2- carboxamide |
| GZ-199 | 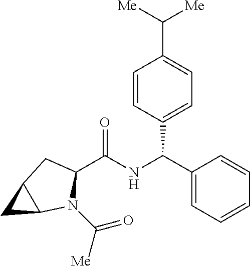 [SMILES: CC(=O)N1[C@H](C(=O)N[C@@H](c2ccccc2)c2ccc(C(C)C)cc2)C[C@@H]2C[C@@H]21] | (1S,3S,5S)-2-acetyl-N-[(S)- phenyl[4-(propan-2- yl)phenyl methyl]-2- azabicyclo[3.1.0]hexane-3- carboxamide |
| GZ-200 | 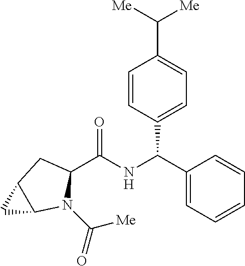 [SMILES: CC(=O)N1[C@@H]2C[C@@H]2C[C@H]1C(=O)N[C@@H](c1ccccc1)c1ccc(C(C)C)cc1] | (1R,3S,5R)-2-acetyl-N-[(S)- phenyl[4-(propan-2- yl)phenyl]methyl]-2- azabicyclo[3.1.0]hexane-3- carboxamide |
| GZ-201 | 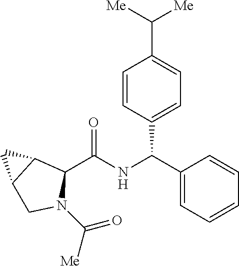 [SMILES: CC(=O)N1C[C@@H]2C[C@@H]2[C@H]1C(=O)N[C@@H](c1ccccc1)c1ccc(C(C)C)cc1] | (1S,2S,5R)-3-acetyl-N-[(S)- phenyl[4-(propan-2- yl)phenyl]methyl]-3- azabicyclo[3.1.0]hexane-2- carboxamide |
| GZ-202 | 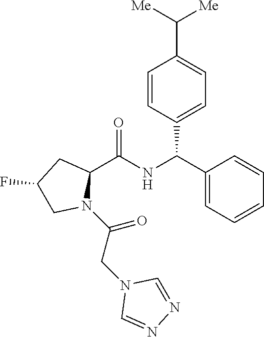 [SMILES: CC(C)c1ccc([C@@H](NC(=O)[C@@H]2C[C@@H](F)CN2C(=O)Cn2cnnc2)c2ccccc2)cc1] | (2RS,4R)-4-fluoro-N-[(S)- phenyl[4-(propan-2- yl)phenyl]methyl]-1-[2-(4H-1,2,4- triazol-4-yl)acetyl]pyrrolidine-2- carboxamide |
| GZ-203 | 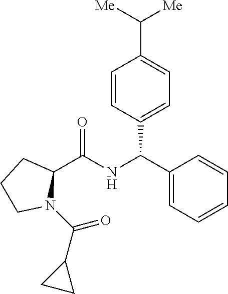 [SMILES: CC(C)c1ccc([C@@H](NC(=O)[C@@H]2CCCN2C(=O)C2CC2)c2ccccc2)cc1] | (2S)-1-cyclopropanecarbonyl-N- [(S)-phenyl[4-(propan-2- yl)phenyl]methyl]pyrrolidine-2- carboxamide |
| GZ-204 | 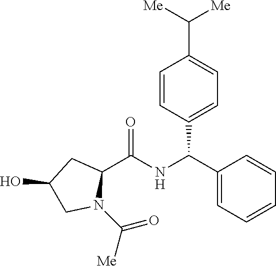 [SMILES: CC(=O)N1C[C@@H](O)C[C@H]1C(=O)N[C@@H](c1ccccc1)c1ccc(C(C)C)cc1] | (2S,4S)-1-acetyl-4-hydroxy-N-[(S)- phenyl[4-(propan-2- yl)phenyl]methyl]pyrrolidine-2- carboxamide |
| GZ-205 | 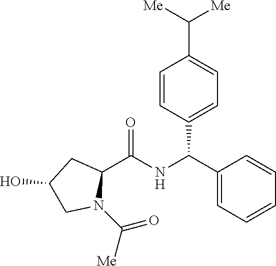 [SMILES: CC(=O)N1C[C@H](O)C[C@H]1C(=O)N[C@@H](c1ccccc1)c1ccc(C(C)C)cc1] | (2S,4R)-1-acetyl-4-hydroxy-N-[(S)- phenyl[4-(propan-2- yl)phenyl]methyl]pyrrolidine-2- carboxamide |
| GZ-206 | 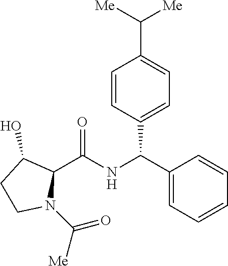 [SMILES: CC(=O)N1CC[C@H](O)[C@H]1C(=O)N[C@@H](c1ccccc1)c1ccc(C(C)C)cc1] | (2S,3S)-1-acetyl-3-hydroxy-N-[(S)- phenyl[4-(propan-2- yl)phenyl]methyl]pyrrolidine-2- carboxamide |
| GZ-207 | 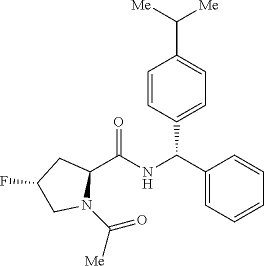 [SMILES: CC(=O)N1C[C@H](F)C[C@H]1C(=O)N[C@@H](c1ccccc1)c1ccc(C(C)C)cc1] | (2S,4R)-1-acetyl-4-fluoro-N-[(S)- phenyl[4-(propan-2- yl)phenyl]methyl]pyrrolidine-2- carboxamide |
| GZ-208 | 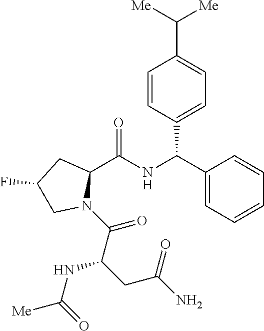 [SMILES: CC(=O)N[C@@H](CC(N)=O)C(=O)N1C[C@H](F)C[C@H]1C(=O)N[C@@H](c1ccccc1)c1ccc(C(C)C)cc1] | (2S,4R)-1-[(2S)-3-carbamoyl-2- acetamidopropanoyl]-4-fluoro-N- [(S)-phenyl[4-(propan-2- yl)phenyl]methyl]pyrrolidine-2- carboxamide |
| GZ-209 | 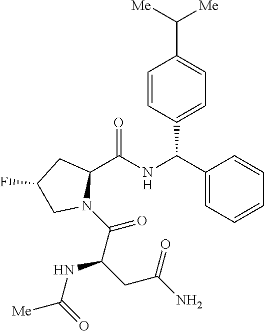 [SMILES: CC(=O)N[C@H](CC(N)=O)C(=O)N1C[C@H](F)C[C@H]1C(=O)N[C@@H](c1ccccc1)c1ccc(C(C)C)cc1] | (2S,4R)-1-[(2R)-3-carbamoyl-2- acetamidopropanoyl]-4-fluoro-N- [(S)-phenyl[4-(propan-2- yl)phenyl methyl]pyrrolidine-2- carboxamide |
| GZ-210 | 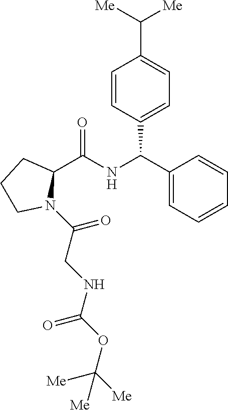 [SMILES: CC(C)c1ccc([C@@H](NC(=O)[C@@H]2CCCN2C(=O)CNC(=O)OC(C)(C)C)c2ccccc2)cc1] | tert-butyl N-{2-oxo-2-[(2S)-2- {[(S)-phenyl[4-(propan-2- yl)phenyl]methyl]carbamoyl} pyrrolidin-1-yl]ethyl} carbamate |
| GZ-211 | 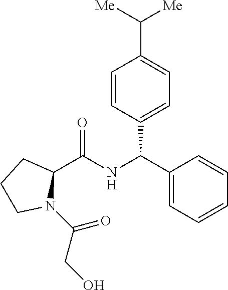 [SMILES: CC(C)c1ccc([C@@H](NC(=O)[C@@H]2CCCN2C(=O)CO)c2ccccc2)cc1] | (2S)-1-(2-hydroxyacetyl)-N-[(S)- phenyl[4-(propan-2- yl)phenyl]methyl]pyrrolidine-2- carboxamide |
| GZ-212 | 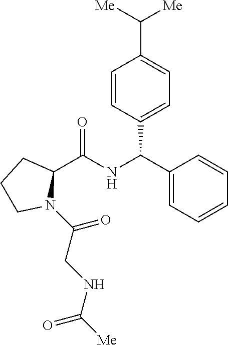 [SMILES: CC(=O)NCC(=O)N1CCC[C@H]1C(=O)N[C@@H](c1ccccc1)c1ccc(C(C)C)cc1] | (2S)-1-(2-acetamidoacetyl)-N-[(S)- phenyl[4-(propan-2- yl)phenyl]methyl]pyrrolidine-2- carboxamide |
| GZ-213 | 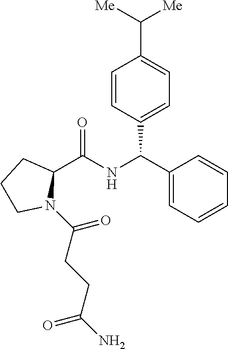 [SMILES: CC(C)c1ccc([C@@H](NC(=O)[C@@H]2CCCN2C(=O)CCC(N)=O)c2ccccc2)cc1] | (2S)-1-(3-carbamoylpropanoyl)-N- [(S)-phenyl[4-(propan-2- yl)phenyl]methyl]pyrrolidine-2- carboxamide |
| GZ-214 | 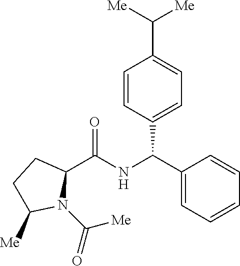 [SMILES: CC(=O)N1[C@@H](C)CC[C@H]1C(=O)N[C@@H](c1ccccc1)c1ccc(C(C)C)cc1] | (2S,5S)-1-acetyl-5-methyl-N-[(S)- phenyl[4-(propan-2- yl)phenyl]methyl]pyrrolidine-2- carboxamide |
| GZ-215 | 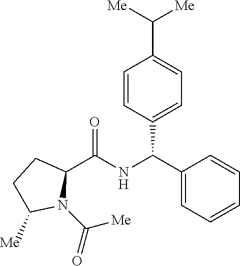 [SMILES: CC(=O)N1[C@H](C)CC[C@H]1C(=O)N[C@@H](c1ccccc1)c1ccc(C(C)C)cc1] | (2S,5R)-1-acetyl-5-methyl-N-[(S)- phenyl[4-(propan-2- yl)phenyl]methyl]pyrrolidine-2- carboxamide |
| GZ-216 | 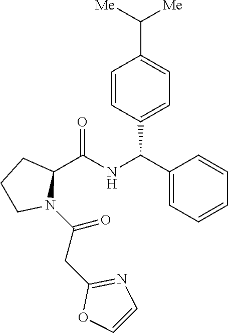 [SMILES: CC(C)c1ccc([C@@H](NC(=O)[C@@H]2CCCN2C(=O)Cc2ncco2)c2ccccc2)cc1] | (2S)-1-[2-(1,3-oxazol-2-yl)acetyl]- N-[(S)-phenyl[4-(propan-2- yl)phenyl]methyl]pyrrolidine-2- carboxamide |
| GZ-217 | 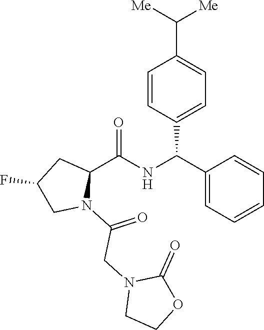 [SMILES: CC(C)c1ccc([C@@H](NC(=O)[C@@H]2C[C@@H](F)CN2C(=O)CN2CCOC2=O)c2ccccc2)cc1] | (2S,4R)-4-fluoro-1-[2-(2-oxo-1,3- oxazolidin-3-yl)acetyl]-N-[(S)- phenyl[4-(propan-2- yl)phenyl]methyl]pyrrolidine-2- carboxamide |
| GZ-218 | 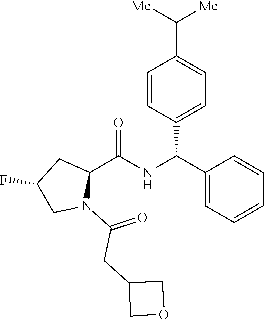 [SMILES: CC(C)c1ccc([C@@H](NC(=O)[C@@H]2C[C@@H](F)CN2C(=O)CC2COC2)c2ccccc2)cc1] | (2S,4R)-4-fluoro-1-[2-(oxetan-3- yl)acetyl]-N-[(S)-phenyl[4-(propan- 2-yl)phenyl]methyl]pyrrolidine-2- carboxamide |
| GZ-219 | 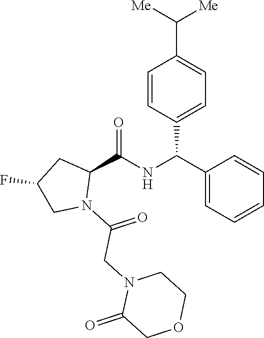 [SMILES: CC(C)c1ccc([C@@H](NC(=O)[C@@H]2C[C@@H](F)CN2C(=O)CN2CCOCC2=O)c2ccccc2)cc1] | (2S,4R)-4-fluoro-1-[2-(3- oxomorpholin-4-yl)acetyl]-N-[(S)- phenyl[4-(propan-2- yl)phenyl]methyl]pyrrolidine-2- carboxamide |
| GZ-220 | 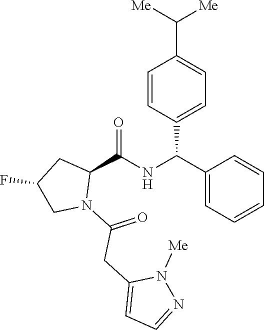 [SMILES: CC(C)c1ccc([C@@H](NC(=O)[C@@H]2C[C@@H](F)CN2C(=O)Cc2ccnn2C)c2ccccc2)cc1] | (2S,4RS)-4-fluoro-1-[2-(1-methyl- 1H-pyrazol-5-yl)acetyl]-N-[(S)- phenyl[4-(propan-2- yl)phenyl]methyl]pyrrolidine-2- carboxamide |
| GZ-221 | 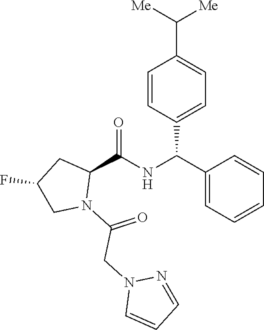 [SMILES: CC(C)c1ccc([C@@H](NC(=O)[C@@H]2C[C@@H](F)CN2C(=O)Cn2cccn2)c2ccccc2)cc1] | (2S,4RS)-4-fluoro-N-[(S)-phenyl[4- (propan-2-yl)phenyl]methyl]-1-[2- (1H-pyrazol-1- yl)acetyl]pyrrolidine-2- carboxamide |
| GZ-222 | 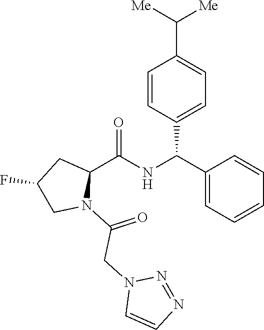 [SMILES: CC(C)c1ccc([C@@H](NC(=O)[C@@H]2C[C@@H](F)CN2C(=O)Cn2ccnn2)c2ccccc2)cc1] | (2RS,4R)-4-fluoro-N-[(S)- phenyl[4-(propan-2- yl)phenyl]methyl]-1-[2-(1H-1,2,3- triazol-1-yl)acetyl]pyrrolidine-2- carboxamide |
| GZ-223 | 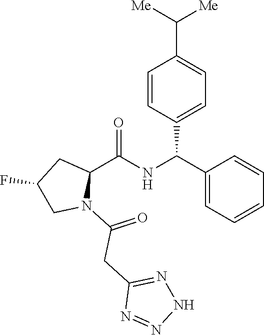 [SMILES: CC(C)c1ccc([C@@H](NC(=O)[C@@H]2C[C@@H](F)CN2C(=O)Cc2nn[nH]n2)c2ccccc2)cc1] | (2RS,4R)-4-fluoro-N-[(S)- phenyl[4-(propan-2- yl)phenyl]methyl]-1-[2-(2H- 1,2,3,4-tetrazol-5- yl)acetyl]pyrrolidine-2- carboxamide |
| GZ-224 | 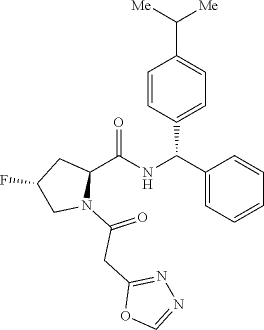 [SMILES: CC(C)c1ccc([C@@H](NC(=O)[C@@H]2C[C@@H](F)CN2C(=O)Cc2nnco2)c2ccccc2)cc1] | (2RS,4R)-4-fluoro-1-[2-(1,3,4- oxadiazol-2-yl)acetyl]-N-[(S)- phenyl[4-(propan-2- yl)phenyl methyl]pyrrolidine-2- carboxamide |
| GZ-225 | 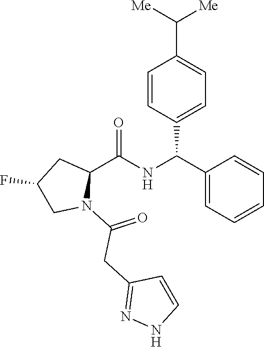 [SMILES: CC(C)c1ccc([C@@H](NC(=O)[C@@H]2C[C@@H](F)CN2C(=O)Cc2cc[nH]n2)c2ccccc2)cc1] | (2RS,4R)-4-fluoro-N-[(S)- phenyl[4-(propan-2- yl)phenyl]methyl]-1-[2-(1H- pyrazol-3-yl)acetyl]pyrrolidine-2- carboxamide |
| GZ-226 | 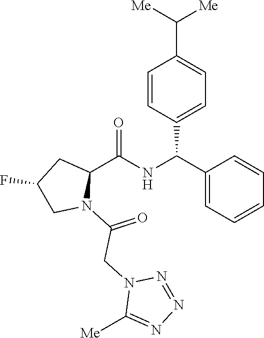 [SMILES: Cc1nnnn1CC(=O)N1C[C@H](F)C[C@H]1C(=O)N[C@@H](c1ccccc1)c1ccc(C(C)C)cc1] | (2RS,4R)-4-fluoro-1-[2-(5-methyl- 1H-1,2,3,4-tetrazol-1-yl)acetyl]-N- [(S)-phenyl[4-(propan-2- yl)phenyl]methyl]pyrrolidine-2- carboxamide |
| GZ-227 | 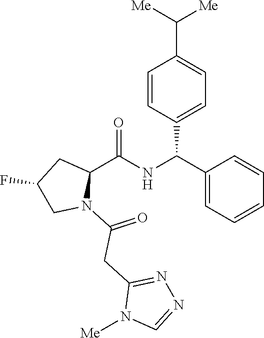 [SMILES: CC(C)c1ccc([C@@H](NC(=O)[C@@H]2C[C@@H](F)CN2C(=O)Cc2nncn2C)c2ccccc2)cc1] | (2RS,4R)-4-fluoro-1-[2-(4-methyl- 4H-1,2,4-triazol-3-yl)acetyl]-N- [(S)-phenyl[4-(propan-2- yl)phenyl]methyl]pyrrolidine-2- carboxamide |
| GZ-228 | 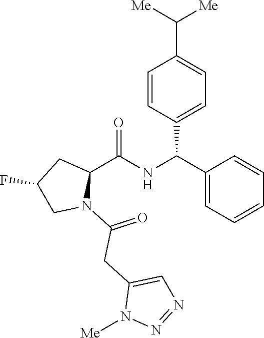 [SMILES: CC(C)c1ccc([C@@H](NC(=O)[C@@H]2C[C@@H](F)CN2C(=O)Cc2cnnn2C)c2ccccc2)cc1] | (2RS,4R)-4-fluoro-1-[2-(1-methyl- 1H-1,2,3-triazol-5-yl)acetyl]-N- [(S)-phenyl[4-(propan-2- yl)phenyl]methyl]pyrrolidine-2- carboxamide |
| GZ-229 | 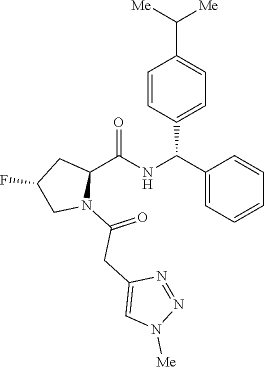 [SMILES: CC(C)c1ccc([C@@H](NC(=O)[C@@H]2C[C@@H](F)CN2C(=O)Cc2cn(C)nn2)c2ccccc2)cc1] | (2RS,4R)-4-fluoro-1-[2-(1-methyl- 1H-1,2,3-triazol-4-yl)acetyl]-N- [(S)-phenyl[4-(propan-2- yl)phenyl]methyl]pyrrolidine-2- carboxamide |
| GZ-230 | 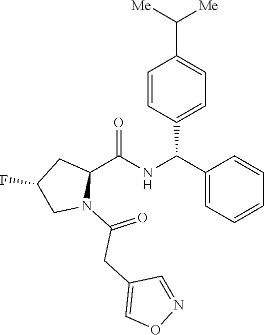 [SMILES: CC(C)c1ccc([C@@H](NC(=O)[C@@H]2C[C@@H](F)CN2C(=O)Cc2cnoc2)c2ccccc2)cc1] | (2S,4R)-4-fluoro-1-[2-(1,2-oxazol- 4-yl)acetyl]-N-[(S)-phenyl[4- (propan-2- yl)phenyl]methyl]pyrrolidine-2- carboxamide |
| GZ-231 | 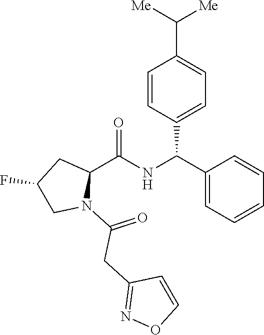 [SMILES: CC(C)c1ccc([C@@H](NC(=O)[C@@H]2C[C@@H](F)CN2C(=O)Cc2ccon2)c2ccccc2)cc1] | (2S,4R)-4-fluoro-1-[2-(1,2-oxazol- 3-yl)acetyl]-N-[(S)-phenyl[4- (propan-2- yl)phenyl]methyl]pyrrolidine-2- carboxamide |
| GZ-232 | 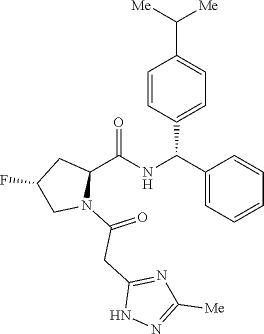 [SMILES: Cc1n[nH]c(CC(=O)N2C[C@H](F)C[C@H]2C(=O)N[C@@H](c2ccccc2)c2ccc(C(C)C)cc2)n1] | (2S,4R)-4-fluoro-1-[2-(3-methyl- 1H-1,2,4-triazol-5-yl)acetyl]-N- [(S)-phenyl[4-(propan-2- yl)phenyl]methyl]pyrrolidine-2- carboxamide |
| GZ-233 | 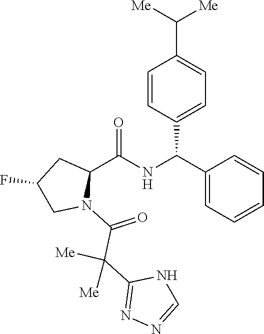 [SMILES: CC(C)c1ccc([C@@H](NC(=O)[C@@H]2C[C@@H](F)CN2C(=O)C(C)(C)c2nnc[nH]2)c2ccccc2)cc1] | (2S,4R)-4-fluoro-1-[2-methyl-2- (1H-1,2,4-triazol-5-yl)propanoyl]- N-[(S)-phenyl[4-(propan-2- yl)phenyl]methyl]pyrrolidine-2- carboxamide |
| GZ-234 | 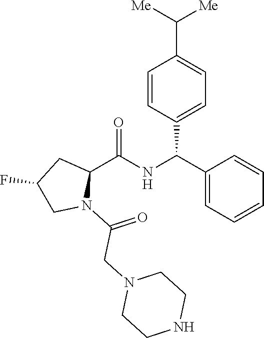 [SMILES: CC(C)c1ccc([C@@H](NC(=O)[C@@H]2C[C@@H](F)CN2C(=O)CN2CCNCC2)c2ccccc2)cc1] | (2S,4R)-4-fluoro-N-[(S)-phenyl[4- (propan-2-yl)phenyl]methyl]-1-[2- (piperazin-1-yl)acetyl]pyrrolidine- 2-carboxamide |
| GZ-235 | 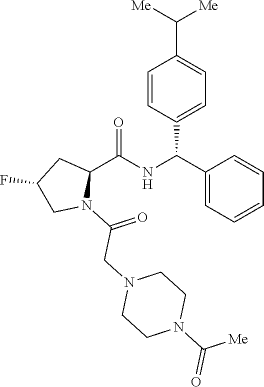 [SMILES: CC(=O)N1CCN(CC(=O)N2C[C@H](F)C[C@H]2C(=O)N[C@@H](c2ccccc2)c2ccc(C(C)C)cc2)CC1] | (2S,4R)-1-[2-(4-acetylpiperazin-1- yl)acetyl]-4-fluoro-N-[(S)- phenyl[4-(propan-2- yl)phenyl]methyl]pyrrolidine-2- carboxamide |
| GZ-236 | 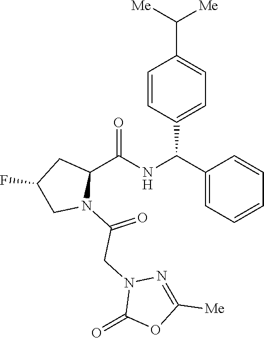 [SMILES: Cc1nn(CC(=O)N2C[C@H](F)C[C@H]2C(=O)N[C@@H](c2ccccc2)c2ccc(C(C)C)cc2)c(=O)o1] | (2S,4R)-4-fluoro-1-[2-(5-methyl-2- oxo-2,3-dihydro-1,3,4-oxadiazol-3- yl)acetyl]-N-[(S)-phenyl[4-(propan- 2-yl)phenyl]methyl]pyrrolidine-2- carboxamide |
| GZ-237 | 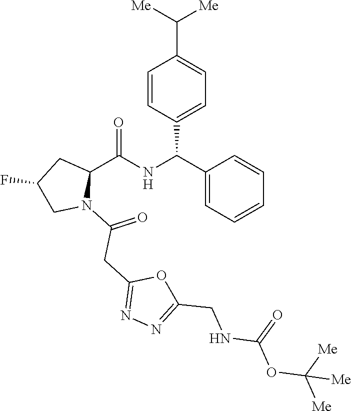 [SMILES: CC(C)c1ccc([C@@H](NC(=O)[C@@H]2C[C@@H](F)CN2C(=O)Cc2nnc(CNC(=O)OC(C)(C)C)o2)c2ccccc2)cc1] | tert-butyl N-[(5-{2-[(2S,4R)-4- fluoro-2-{[(S)-phenyl[4-(propan-2- yl)phenyl methyl] carbamoyl} pyrrolidin-1-yl]-2-oxoethyl}-1,3,4- oxadiazol-2-yl)methyl]carbamate |
| GZ-238 | 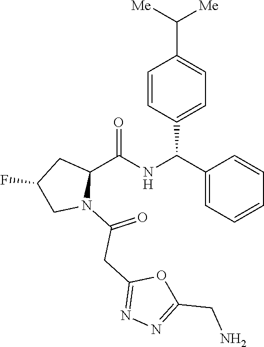 [SMILES: CC(C)c1ccc([C@@H](NC(=O)[C@@H]2C[C@@H](F)CN2C(=O)Cc2nnc(CN)o2)c2ccccc2)cc1] | (2S,4R)-1-{2-[5-(aminomethyl)- 1,3,4-oxadiazol-2-yl]acetyl}-4- fluoro-N-[(S)-phenyl[4-(propan-2- yl)phenyl]methyl]pyrrolidine-2- carboxamide |
| GZ-239 | 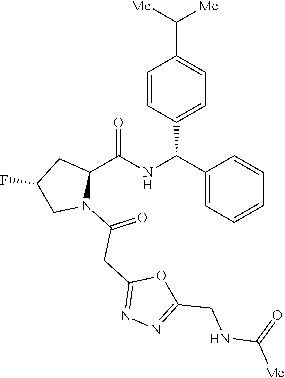 [SMILES: CC(=O)NCc1nnc(CC(=O)N2C[C@H](F)C[C@H]2C(=O)N[C@@H](c2ccccc2)c2ccc(C(C)C)cc2)o1] | (2S,4R)-1-{2-[5- (acetamidomethyl)-1,3,4-oxadiazol- 2-yl]acetyl}-4-fluoro-N-[(S)- phenyl[4-(propan-2- yl)phenyl methyl]pyrrolidine-2- carboxamide |
| GZ-240 | 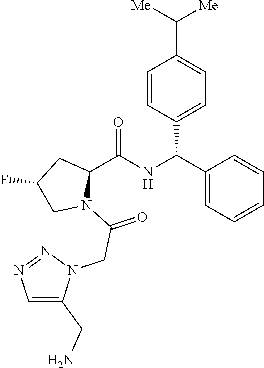 [SMILES: CC(C)c1ccc([C@@H](NC(=O)[C@@H]2C[C@@H](F)CN2C(=O)Cn2nncc2CN)c2ccccc2)cc1] | (2S,4R)-1-{2-[5-(aminomethyl)- 1H-1,2,3-triazol-1-yl]acetyl}-4- fluoro-N-[(S)-phenyl[4-(propan-2- yl)phenyl methyl]pyrrolidine-2- carboxamide |
| GZ-241 | 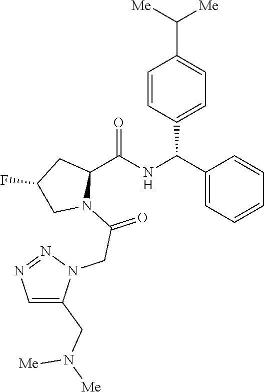 [SMILES: CC(C)c1ccc([C@@H](NC(=O)[C@@H]2C[C@@H](F)CN2C(=O)Cn2nncc2CN(C)C)c2ccccc2)cc1] | (2S,4R)-1-(2-{5- [(dimethylamino)methyl]-1H-1,2,3- triazol-1-yl} acetyl)-4-fluoro-N- [(S)-phenyl[4-(propan-2- yl)phenyl]methyl]pyrrolidine-2- carboxamide |
| GZ-242 | 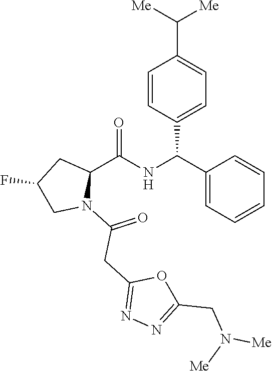 [SMILES: CC(C)c1ccc([C@@H](NC(=O)[C@@H]2C[C@@H](F)CN2C(=O)Cc2nnc(CN(C)C)o2)c2ccccc2)cc1] | (2S,4R)-1-(2-{5- [(dimethylamino)methyl]-1,3,4- oxadiazol-2-yl} acetyl)-4-fluoro-N- [(S)-phenyl[4-(propan-2- yl)phenyl]methyl]pyrrolidine-2- carboxamide |
| GZ-243 | 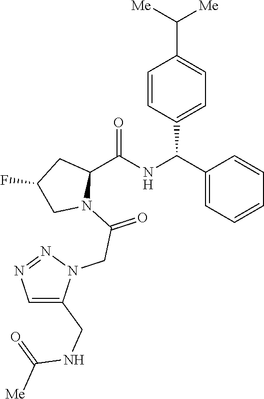 [SMILES: CC(=O)NCc1cnnn1CC(=O)N1C[C@H](F)C[C@H]1C(=O)N[C@@H](c1ccccc1)c1ccc(C(C)C)cc1] | (2S,4R)-1-{2-[5- (acetamidomethyl)-1H-1,2,3- triazol-1-yl]acetyl}-4-fluoro-N- [(S)-phenyl[4-(propan-2- yl)phenyl]methyl]pyrrolidine-2- carboxamide |
| GZ-244 | 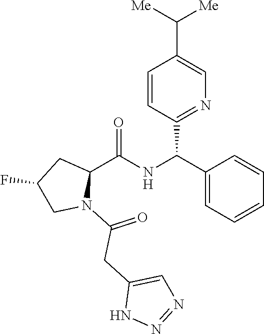 [SMILES: CC(C)c1ccc([C@@H](NC(=O)[C@@H]2C[C@@H](F)CN2C(=O)Cc2cnn[nH]2)c2ccccc2)nc1] | (2S,4R)-1-(2-(1H-1,2,3-triazol-5- yl)acetyl)-4-fluoro-N-((S)-(5- isopropylpyridin-2- yl)(phenyl)methyl)pyrrolidine-2- carboxamide |
| GZ-245 | 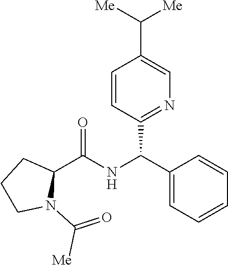 [SMILES: CC(=O)N1CCC[C@H]1C(=O)N[C@@H](c1ccccc1)c1ccc(C(C)C)cn1] | (2S)-1-acetyl-N-[(S)-phenyl[5- (propan-2-yl)pyridin-2- yl]methyl]pyrrolidine-2- carboxamide |
| GZ-246 | 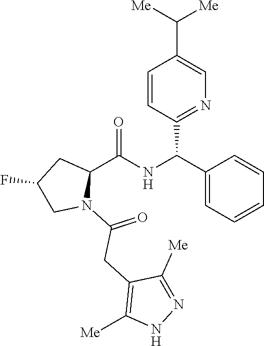 [SMILES: Cc1n[nH]c(C)c1CC(=O)N1C[C@H](F)C[C@H]1C(=O)N[C@@H](c1ccccc1)c1ccc(C(C)C)cn1] | (2S,4R)-1-[2-(3,5-dimethyl-1H- pyrazol-4-yl)acetyl]-4-fluoro-N- [(S)-phenyl[5-(propan-2-yl)pyridin- 2-yl]methyl]pyrrolidine-2- carboxamide |
| GZ-247 | 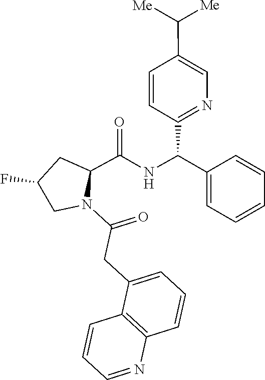 [SMILES: CC(C)c1ccc([C@@H](NC(=O)[C@@H]2C[C@@H](F)CN2C(=O)Cc2cccc3ncccc23)c2ccccc2)nc1] | (2S,4R)-4-fluoro-N-[(S)-phenyl[5- (propan-2-yl)pyridin-2-yl]methyl]- 1-[2-(quinolin-5- yl)acetyl]pyrrolidine-2- carboxamide |
| GZ-248 | 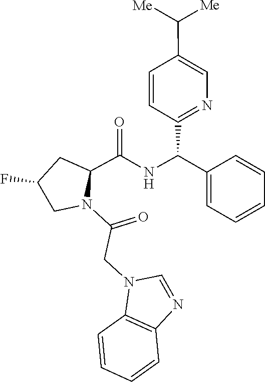 [SMILES: CC(C)c1ccc([C@@H](NC(=O)[C@@H]2C[C@@H](F)CN2C(=O)Cn2cnc3ccccc32)c2ccccc2)nc1] | (2S,4R)-1-[2-(1H-1,3-benzodiazol- 1-yl)acetyl]-4-fluoro-N-[(S)- phenyl[5-(propan-2-yl)pyridin-2- yl]methyl]pyrrolidine-2- carboxamide |
| GZ-249 | 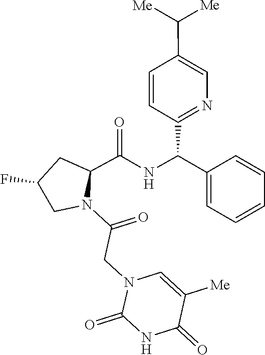 [SMILES: Cc1cn(CC(=O)N2C[C@H](F)C[C@H]2C(=O)N[C@@H](c2ccccc2)c2ccc(C(C)C)cn2)c(=O)[nH]c1=O] | (2S,4R)-4-fluoro-1-[2-(5-methyl- 2,4-dioxo-1,2,3,4- tetrahydropyrimidin-1-yl)acetyl]-N- [(S)-phenyl[5-(propan-2-yl)pyridin- 2-yl]methyl]pyrrolidine-2- carboxamide |
| GZ-250 | 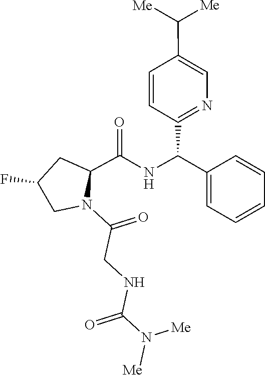 [SMILES: CC(C)c1ccc([C@@H](NC(=O)[C@@H]2C[C@@H](F)CN2C(=O)CNC(=O)N(C)C)c2ccccc2)nc1] | (2S,4R)-1-{2- [(dimethylcarbamoyl)amino]acetyl}- 4-fluoro-N-[(S)-phenyl[5-(propan- 2-yl)pyridin-2- yl]methyl]pyrrolidine-2- carboxamide |
| GZ-251 | 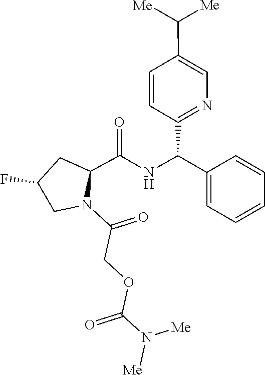 [SMILES: CC(C)c1ccc([C@@H](NC(=O)[C@@H]2C[C@@H](F)CN2C(=O)COC(=O)N(C)C)c2ccccc2)nc1] | 2-[(2S,4R)-4-fluoro-2-{[(S)- phenyl[5-(propan-2-yl)pyridin-2- yl]methyl]carbamoyl} pyrrolidin-1- yl]-2-oxoethyl N,N- dimethylcarbamate |
| GZ-252 | 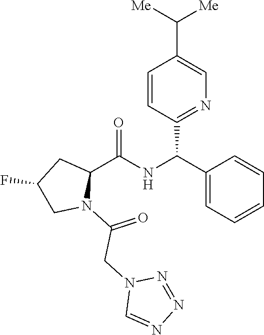 [SMILES: CC(C)c1ccc([C@@H](NC(=O)[C@@H]2C[C@@H](F)CN2C(=O)Cn2cnnn2)c2ccccc2)nc1] | (2S,4R)-4-fluoro-N-[(S)-phenyl[5- (propan-2-yl)pyridin-2-yl]methyl]- 1-[2-(1H-1,2,3,4-tetrazol-1- yl)acetyl]pyrrolidine-2- carboxamide |
| GZ-253 | 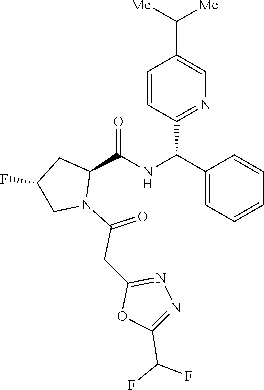 [SMILES: CC(C)c1ccc([C@@H](NC(=O)[C@@H]2C[C@@H](F)CN2C(=O)Cc2nnc(C(F)F)o2)c2ccccc2)nc1] | (2S,4R)-1-{2-[5-(difluoromethyl)- 1,3,4-oxadiazol-2-yl]acetyl}-4- fluoro-N-[(S)-phenyl[5-(propan-2- yl)pyridin-2-yl]methyl]pyrrolidine- 2-carboxamide |
| GZ-254 | 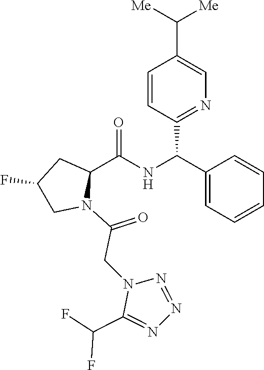 [SMILES: CC(C)c1ccc([C@@H](NC(=O)[C@@H]2C[C@@H](F)CN2C(=O)Cn2nnnc2C(F)F)c2ccccc2)nc1] | (2S,4R)-1-{2-[5-(difluoromethyl)- 1H-1,2,3,4-tetrazol-1-yl]acetyl}-4- fluoro-N-[(S)-phenyl[5-(propan-2- yl)pyridin-2-yl]methyl]pyrrolidine- 2-carboxamide |
| GZ-255 | 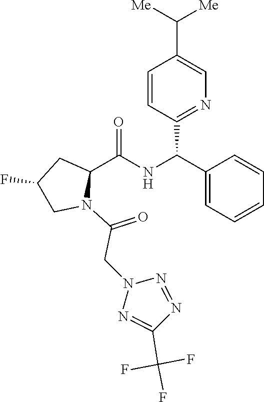 [SMILES: CC(C)c1ccc([C@@H](NC(=O)[C@@H]2C[C@@H](F)CN2C(=O)Cn2nnc(C(F)(F)F)n2)c2ccccc2)nc1] | (2S,4R)-4-fluoro-N-[(S)-phenyl[5- (propan-2-yl)pyridin-2-yl]methyl]- 1-{2-[5-(trifluoromethyl)-2H- 1,2,3,4-tetrazol-2- yl]acetyl} pyrrolidine-2- carboxamide |
| GZ-256 | 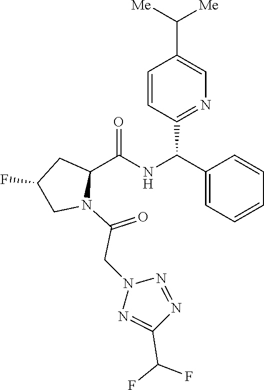 [SMILES: CC(C)c1ccc([C@@H](NC(=O)[C@@H]2C[C@@H](F)CN2C(=O)Cn2nnc(C(F)F)n2)c2ccccc2)nc1] | (2S,4R)-1-{2-[5-(difluoromethyl)- 2H-1,2,3,4-tetrazol-2-yl]acetyl}-4- fluoro-N-[(S)-phenyl[5-(propan-2- yl)pyridin-2-yl]methyl]pyrrolidine- 2-carboxamide |
| GZ-257 | 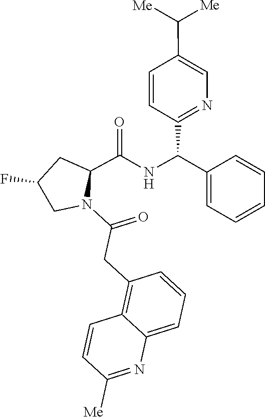 [SMILES: Cc1ccc2c(CC(=O)N3C[C@H](F)C[C@H]3C(=O)N[C@@H](c3ccccc3)c3ccc(C(C)C)cn3)cccc2n1] | (2S,4R)-4-fluoro-1-[2-(2- methylquinolin-5-yl)acetyl]-N-[(S)- phenyl[5-(propan-2-yl)pyridin-2- yl]methyl]pyrrolidine-2- carboxamide |
| GZ-258 | 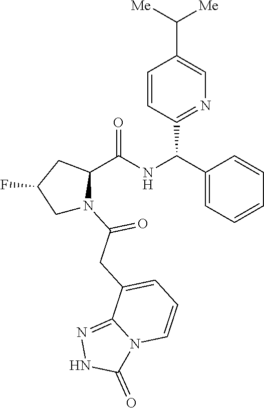 [SMILES: CC(C)c1ccc([C@@H](NC(=O)[C@@H]2C[C@@H](F)CN2C(=O)Cc2cccn3c(=O)[nH]nc23)c2ccccc2)nc1] | (2S,4R)-4-fluoro-1-(2-{3-oxo- 2H,3H-[1,2,4]triazolo[4,3- alpyridin-8-yl} acetyl)-N-[(S)- phenyl[5-(propan-2-yl)pyridin-2- yl]methyl]pyrrolidine-2- carboxamide |
| GZ-259 | 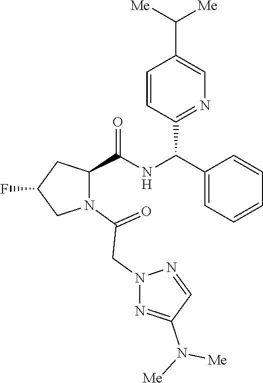 [SMILES: CC(C)c1ccc([C@@H](NC(=O)[C@@H]2C[C@@H](F)CN2C(=O)Cn2ncc(N(C)C)n2)c2ccccc2)nc1] | (2S,4R)-1-{2-[4-(dimethylamino)- 2H-1,2,3-triazol-2-yl]acetyl}-4- fluoro-N-[(S)-phenyl[5-(propan-2- yl)pyridin-2-yl]methyl]pyrrolidine- 2-carboxamide |
| GZ-260 | 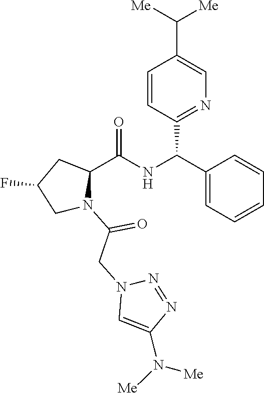 [SMILES: CC(C)c1ccc([C@@H](NC(=O)[C@@H]2C[C@@H](F)CN2C(=O)Cn2cc(N(C)C)nn2)c2ccccc2)nc1] | (2S,4R)-1-{2-[4-(dimethylamino)- 1H-1,2,3-triazol-1-yl]acetyl}-4- fluoro-N-[(S)-phenyl[5-(propan-2- yl)pyridin-2-yl]methyl]pyrrolidine- 2-carboxamide |
| GZ-261 | 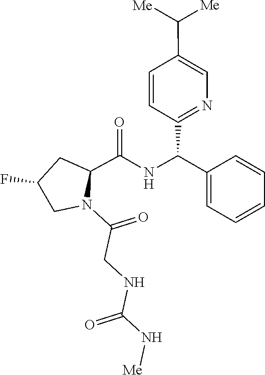 [SMILES: CNC(=O)NCC(=O)N1C[C@H](F)C[C@H]1C(=O)N[C@@H](c1ccccc1)c1ccc(C(C)C)cn1] | (2S,4R)-4-fluoro-1-{2- [(methylcarbamoyl)amino]acetyl}- N-[(S)-phenyl[5-(propan-2- yl)pyridin-2-yl]methyl]pyrrolidine- 2-carboxamide |
| GZ-262 | 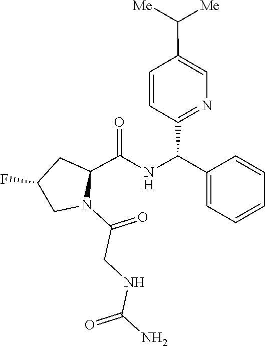 [SMILES: CC(C)c1ccc([C@@H](NC(=O)[C@@H]2C[C@@H](F)CN2C(=O)CNC(N)=O)c2ccccc2)nc1] | (2S,4R)-1-[2- (carbamoylamino)acetyl]-4-fluoro- N-[(S)-phenyl[5-(propan-2- yl)pyridin-2-yl]methyl]pyrrolidine- 2-carboxamide |
| GZ-263 | 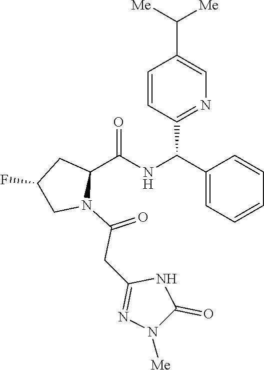 [SMILES: CC(C)c1ccc([C@@H](NC(=O)[C@@H]2C[C@@H](F)CN2C(=O)Cc2nn(C)c(=O)[nH]2)c2ccccc2)nc1] | (2S,4R)-4-fluoro-1-[2-(1-methyl-5- oxo-4,5-dihydro-1H-1,2,4-triazol-3- yl)acetyl]-N-[(S)-phenyl[5-(propan- 2-yl)pyridin-2- yl]methyl]pyrrolidine-2- carboxamide |
| GZ-264 | 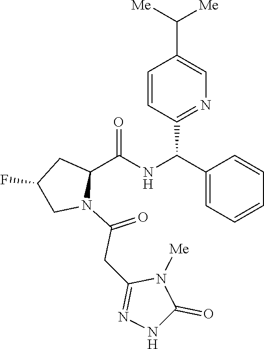 [SMILES: CC(C)c1ccc([C@@H](NC(=O)[C@@H]2C[C@@H](F)CN2C(=O)Cc2n[nH]c(=O)n2C)c2ccccc2)nc1] | (2S,4R)-4-fluoro-1-[2-(4-methyl-5- oxo-4,5-dihydro-1H-1,2,4-triazol-3- yl)acetyl]-N-[(S)-phenyl[5-(propan- 2-yl)pyridin-2- yl\|methyl]pyrrolidine-2- carboxamide |
| GZ-265 | 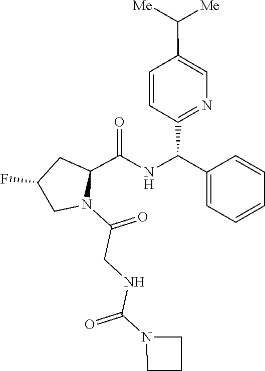 [SMILES: CC(C)c1ccc([C@@H](NC(=O)[C@@H]2C[C@@H](F)CN2C(=O)CNC(=O)N2CCC2)c2ccccc2)nc1] | (2S,4R)-1-{2-[(azetidine-1- carbonyl)amino]acetyl}-4-fluoro- N-[(S)-phenyl[5-(propan-2- yl)pyridin-2-yl]methyl]pyrrolidine- 2-carboxamide |
| GZ-266 | 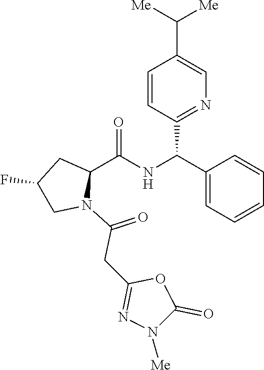 [SMILES: CC(C)c1ccc([C@@H](NC(=O)[C@@H]2C[C@@H](F)CN2C(=O)Cc2nn(C)c(=O)o2)c2ccccc2)nc1] | (2S,4R)-4-fluoro-1-[2-(4-methyl-5- oxo-4,5-dihydro-1,3,4-oxadiazol-2- yl)acetyl]-N-[(S)-phenyl[5-(propan- 2-yl)pyridin-2- yl]methyl]pyrrolidine-2- carboxamide |
| GZ-267 | 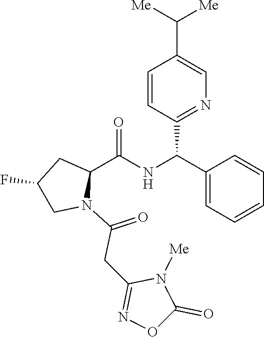 [SMILES: CC(C)c1ccc([C@@H](NC(=O)[C@@H]2C[C@@H](F)CN2C(=O)Cc2noc(=O)n2C)c2ccccc2)nc1] | (2S,4R)-4-fluoro-1-[2-(4-methyl-5- oxo-4,5-dihydro-1,2,4-oxadiazol-3- yl)acetyl]-N-[(S)-phenyl[5-(propan- 2-yl)pyridin-2- yl]methyl]pyrrolidine-2- carboxamide |
| GZ-268 | 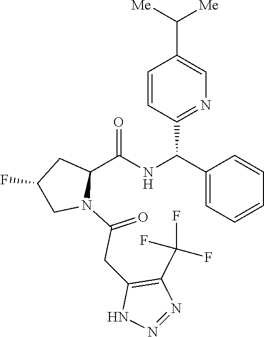 [SMILES: CC(C)c1ccc([C@@H](NC(=O)[C@@H]2C[C@@H](F)CN2C(=O)Cc2[nH]nnc2C(F)(F)F)c2ccccc2)nc1] | (2S,4R)-4-fluoro-N-[(S)-phenyl[5- (propan-2-yl)pyridin-2-yl]methyl]- 1-{2-[4-(trifluoromethyl)-1H-1,2,3- triazol-5-yl]acetyl} pyrrolidine-2- carboxamide |
| GZ-269 | 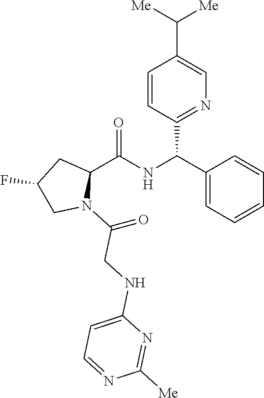 [SMILES: Cc1nccc(NCC(=O)N2C[C@H](F)C[C@H]2C(=O)N[C@@H](c2ccccc2)c2ccc(C(C)C)cn2)n1] | (2S,4R)-4-fluoro-1-{2-[(2- methylpyrimidin-4- yl)amino]acetyl}-N-[(S)-phenyl[5- (propan-2-yl)pyridin-2- yl]methyl]pyrrolidine-2- carboxamide |
| GZ-270 | 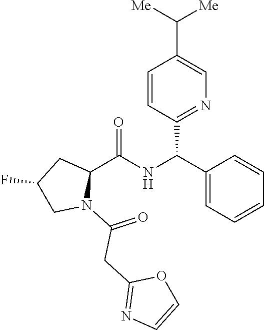 [SMILES: CC(C)c1ccc([C@@H](NC(=O)[C@@H]2C[C@@H](F)CN2C(=O)Cc2ncco2)c2ccccc2)nc1] | (2S,4R)-4-fluoro-1-[2-(1,3-oxazol- 2-yl)acetyl]-N-[(S)-phenyl[5- (propan-2-yl)pyridin-2- yl]methyl]pyrrolidine-2- carboxamide |
| GZ-271 | 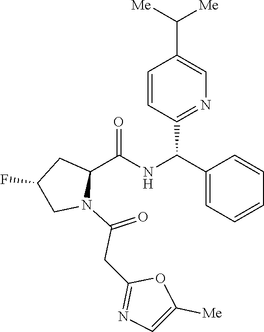 [SMILES: Cc1cnc(CC(=O)N2C[C@H](F)C[C@H]2C(=O)N[C@@H](c2ccccc2)c2ccc(C(C)C)cn2)o1] | (2S,4R)-4-fluoro-1-[2-(5-methyl- 1,3-oxazol-2-yl)acetyl]-N-[(S)- phenyl[5-(propan-2-yl)pyridin-2- yl]methyl]pyrrolidine-2- carboxamide |
| GZ-272 | 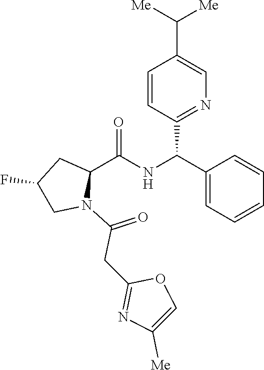 [SMILES: Cc1coc(CC(=O)N2C[C@H](F)C[C@H]2C(=O)N[C@@H](c2ccccc2)c2ccc(C(C)C)cn2)n1] | (2S,4R)-4-fluoro-1-[2-(4-methyl- 1,3-oxazol-2-yl)acetyl]-N-[(S)- phenyl[5-(propan-2-yl)pyridin-2- yl\|methyl]pyrrolidine-2- carboxamide |
| GZ-273 | 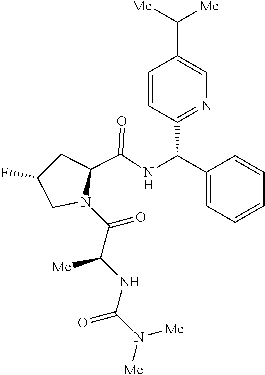 [SMILES: CC(C)c1ccc([C@@H](NC(=O)[C@@H]2C[C@@H](F)CN2C(=O)[C@H](C)NC(=O)N(C)C)c2ccccc2)nc1] | (2S,4R)-1-[(2S)-2- [(dimethylcarbamoyl)amino] propanoyl]-4-fluoro-N-[(S)-phenyl[5- (propan-2-yl)pyridin-2- yl]methyl]pyrrolidine-2- carboxamide |
| GZ-274 | 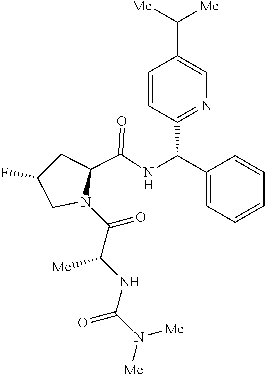 [SMILES: CC(C)c1ccc([C@@H](NC(=O)[C@@H]2C[C@@H](F)CN2C(=O)[C@@H](C)NC(=O)N(C)C)c2ccccc2)nc1] | (2S,4R)-1-[(2R)-2- [(dimethylcarbamoyl)amino] propanoyl]-4-fluoro-N-[(S)-phenyl[5- (propan-2-yl)pyridin-2- yl]methyl]pyrrolidine-2- carboxamide |
| GZ-275 | 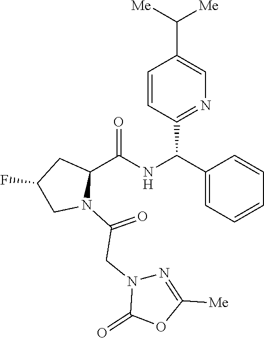 [SMILES: Cc1nn(CC(=O)N2C[C@H](F)C[C@H]2C(=O)N[C@@H](c2ccccc2)c2ccc(C(C)C)cn2)c(=O)o1] | (2S,4R)-4-fluoro-1-[2-(5-methyl-2- oxo-2,3-dihydro-1,3,4-oxadiazol-3- yl)acetyl]-N-[(S)-phenyl[5-(propan- 2-yl)pyridin-2- yl]methyl]pyrrolidine-2- carboxamide |
| GZ-276 | 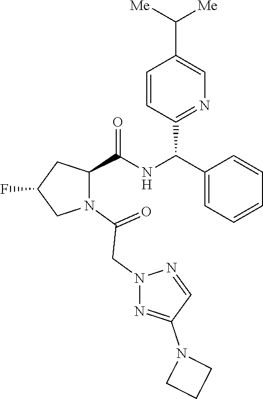 [SMILES: CC(C)c1ccc([C@@H](NC(=O)[C@@H]2C[C@@H](F)CN2C(=O)Cn2ncc(N3CCC3)n2)c2ccccc2)nc1] | (2S,4R)-1-{2-[4-(azetidin-1-yl)-2H- 1,2,3-triazol-2-yl]acetyl}-4-fluoro- N-[(S)-phenyl[5-(propan-2- yl)pyridin-2-yl]methyl]pyrrolidine- 2-carboxamide |
| GZ-277 | 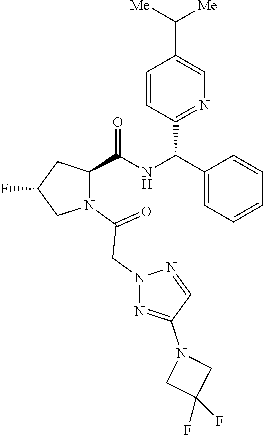 [SMILES: CC(C)c1ccc([C@@H](NC(=O)[C@@H]2C[C@@H](F)CN2C(=O)Cn2ncc(N3CC(F)(F)C3)n2)c2ccccc2)nc1] | (2S,4R)-1-{2-[4-(3,3- difluoroazetidin-1-yl)-2H-1,2,3- triazol-2-yl]acetyl }-4-fluoro-N- [(S)-phenyl[5-(propan-2-yl)pyridin- 2-yl]methyl]pyrrolidine-2- carboxamide |
| GZ-278 | 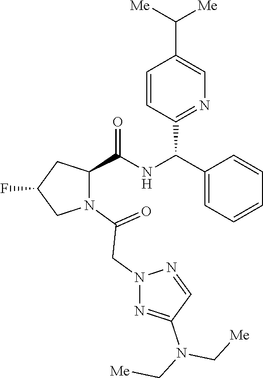 [SMILES: CCN(CC)c1cnn(CC(=O)N2C[C@H](F)C[C@H]2C(=O)N[C@@H](c2ccccc2)c2ccc(C(C)C)cn2)n1] | (2S,4R)-1-{2-[4-(diethylamino)- 2H-1,2,3-triazol-2-yl]acetyl}-4- fluoro-N-[(S)-phenyl[5-(propan-2- yl)pyridin-2-yl]methyl]pyrrolidine- 2-carboxamide |
| GZ-279 | 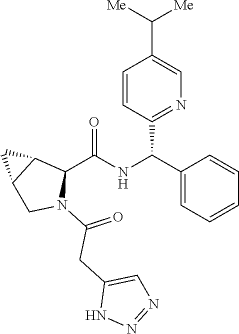 [SMILES: CC(C)c1ccc([C@@H](NC(=O)[C@@H]2[C@H]3C[C@H]3CN2C(=O)Cc2cnn[nH]2)c2ccccc2)nc1] | (1S,2S,5R)-N-[(S)-phenyl[5- (propan-2-yl)pyridin-2-yl]methyl]- 3-[2-(1H-1,2,3-triazol-5-yl)acetyl]- 3-azabicyclo[3.1.0]hexane-2- carboxamide |
| GZ-280 | 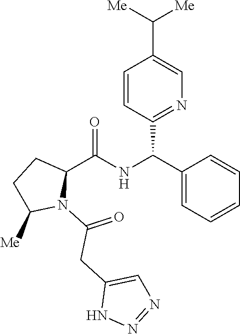 [SMILES: CC(C)c1ccc([C@@H](NC(=O)[C@@H]2CC[C@H](C)N2C(=O)Cc2cnn[nH]2)c2ccccc2)nc1] | (2S,5S)-5-methyl-N-[(S)-phenyl[5- (propan-2-yl)pyridin-2-yl]methyl]- 1-[2-(1H-1,2,3-triazol-5- yl)acetyl]pyrrolidine-2- carboxamide |
| GZ-281 | 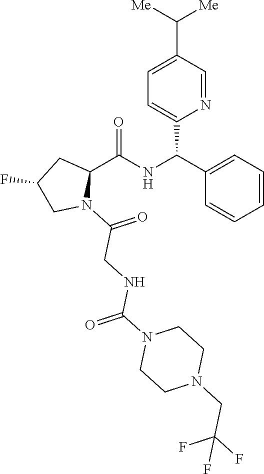 [SMILES: CC(C)c1ccc([C@@H](NC(=O)[C@@H]2C[C@@H](F)CN2C(=O)CNC(=O)N2CCN(CC(F)(F)F)CC2)c2ccccc2)nc1] | N-{2-[(2S,4R)-4-fluoro-2-{[(S)- phenyl[5-(propan-2-yl)pyridin-2- yl]methyl]carbamoyl}pyrrolidin-1- yl]-2-oxoethyl}-4-(2,2,2- trifluoroethyl)piperazine-1- carboxamide |
| GZ-282 | 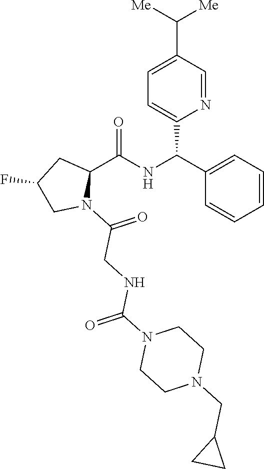 [SMILES: CC(C)c1ccc([C@@H](NC(=O)[C@@H]2C[C@@H](F)CN2C(=O)CNC(=O)N2CCN(CC3CC3)CC2)c2ccccc2)nc1] | 4-(cyclopropylmethyl)-N-{2- [(2S,4R)-4-fluoro-2-{[(S)- phenyl[5-(propan-2-yl)pyridin-2- yl\|methyl\|carbamoyl}pyrrolidin-1- yl]-2-oxoethyl} piperazine-1- carboxamide |
| GZ-283 | 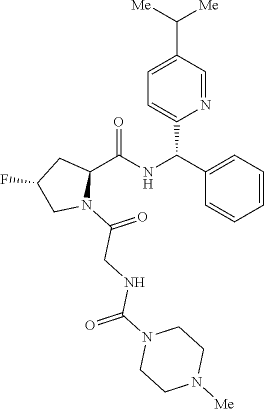 [SMILES: CC(C)c1ccc([C@@H](NC(=O)[C@@H]2C[C@@H](F)CN2C(=O)CNC(=O)N2CCN(C)CC2)c2ccccc2)nc1] | N-{2-[(2S,4R)-4-fluoro-2-{[(S)- phenyl[5-(propan-2-yl)pyridin-2- yl]methyl]carbamoyl} pyrrolidin-1- yl]-2-oxoethyl}-4- methylpiperazine-1-carboxamide |
| GZ-284 | 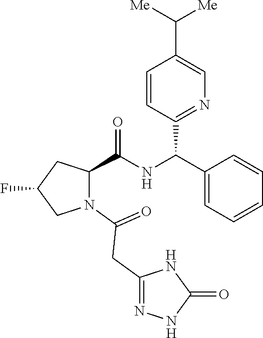 [SMILES: CC(C)c1ccc([C@@H](NC(=O)[C@@H]2C[C@@H](F)CN2C(=O)Cc2n[nH]c(=O)[nH]2)c2ccccc2)nc1] | (2S,4R)-4-fluoro-1-[2-(5-oxo-4,5- dihydro-1H-1,2,4-triazol-3- yl)acetyl]-N-[(S)-phenyl[5-(propan- 2-yl)pyridin-2- yl]methyl]pyrrolidine-2- carboxamide |
| GZ-285 | 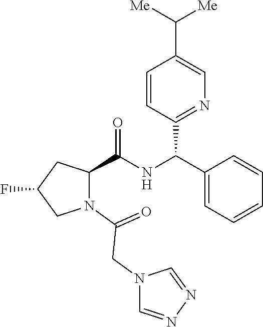 [SMILES: CC(C)c1ccc([C@@H](NC(=O)[C@@H]2C[C@@H](F)CN2C(=O)Cn2cnnc2)c2ccccc2)nc1] | (2S,4R)-4-fluoro-N-[(S)-phenyl[5- (propan-2-yl)pyridin-2-yl]methyl]- 1-[2-(4H-1,2,4-triazol-4- yl)acetyl]pyrrolidine-2- carboxamide |
| GZ-286 | 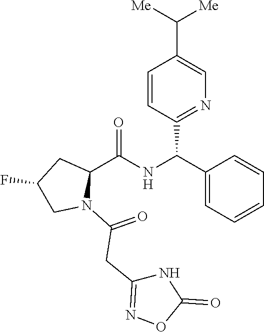 [SMILES: CC(C)c1ccc([C@@H](NC(=O)[C@@H]2C[C@@H](F)CN2C(=O)Cc2noc(=O)[nH]2)c2ccccc2)nc1] | (2S,4R)-4-fluoro-1-[2-(5-oxo-4,5- dihydro-1,2,4-oxadiazol-3- yl)acetyl]-N-[(S)-phenyl[5-(propan- 2-yl)pyridin-2- yl]methyl]pyrrolidine-2- carboxamide |
| GZ-287 | 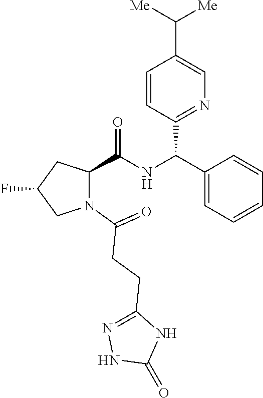 [SMILES: CC(C)c1ccc([C@@H](NC(=O)[C@@H]2C[C@@H](F)CN2C(=O)CCc2n[nH]c(=O)[nH]2)c2ccccc2)nc1] | (2S,4R)-4-fluoro-1-[3-(5-oxo-4,5- dihydro-1H-1,2,4-triazol-3- yl)propanoyl]-N-[(S)-phenyl[5- (propan-2-yl)pyridin-2- yl]methyl]pyrrolidine-2- carboxamide |
| GZ-288 | 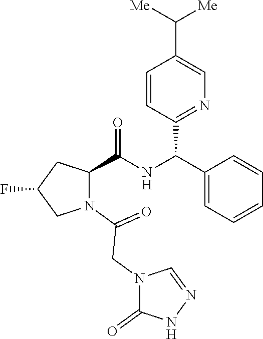 [SMILES: CC(C)c1ccc([C@@H](NC(=O)[C@@H]2C[C@@H](F)CN2C(=O)Cn2cn[nH]c2=O)c2ccccc2)nc1] | (2S,4R)-4-fluoro-1-[2-(5-oxo-4,5- dihydro-1H-1,2,4-triazol-4- yl)acetyl]-N-[(S)-phenyl[5-(propan- 2-yl)pyridin-2- yl]methyl]pyrrolidine-2- carboxamide |
| GZ-289 | 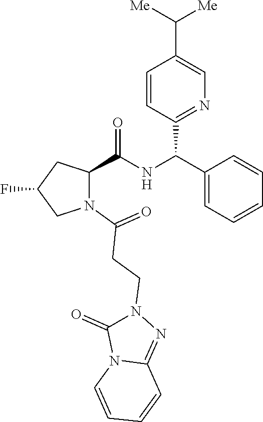 [SMILES: CC(C)c1ccc([C@@H](NC(=O)[C@@H]2C[C@@H](F)CN2C(=O)CCn2nc3ccccn3c2=O)c2ccccc2)nc1] | (2S,4R)-4-fluoro-1-(3-{3-oxo- 2H,3H-[1,2,4]triazolo[4,3- alpyridin-2-yl} propanoyl)-N-[(S)- phenyl[5-(propan-2-yl)pyridin-2- yl]methyl]pyrrolidine-2- carboxamide |
| GZ-290 | 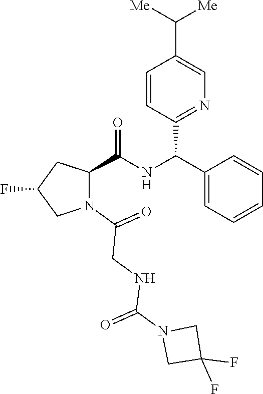 [SMILES: CC(C)c1ccc([C@@H](NC(=O)[C@@H]2C[C@@H](F)CN2C(=O)CNC(=O)N2CC(F)(F)C2)c2ccccc2)nc1] | (2S,4R)-1-{2-[(3,3- difluoroazetidine-1- carbonyl)amino]acetyl}-4-fluoro- N-[(S)-phenyl[5-(propan-2- yl)pyridin-2-yl\|methyl]pyrrolidine- 2-carboxamide |
| GZ-291 | 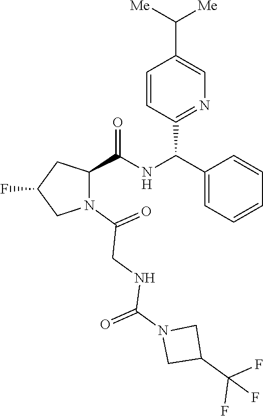 [SMILES: CC(C)c1ccc([C@@H](NC(=O)[C@@H]2C[C@@H](F)CN2C(=O)CNC(=O)N2CC(C(F)(F)F)C2)c2ccccc2)nc1] | (2S,4R)-4-fluoro-N-[(S)-phenyl[5- (propan-2-yl)pyridin-2-yl]methyl]- 1-(2-{[3-(trifluoromethyl)azetidine- 1- carbonyl]amino} acetyl)pyrrolidine- 2-carboxamide |
| GZ-292 | 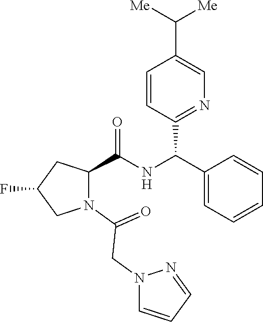 [SMILES: CC(C)c1ccc([C@@H](NC(=O)[C@@H]2C[C@@H](F)CN2C(=O)Cn2cccn2)c2ccccc2)nc1] | (2S,4R)-4-fluoro-N-[(S)-phenyl[5- (propan-2-yl)pyridin-2-yl]methyl]- 1-[2-(1H-pyrazol-1- yl)acetyl]pyrrolidine-2- carboxamide |
| GZ-293 | 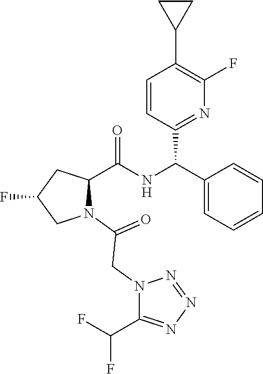 [SMILES: O=C(N[C@@H](c1ccccc1)c1ccc(C2CC2)c(F)n1)[C@@H]1C[C@@H](F)CN1C(=O)Cn1nnnc1C(F)F] | (2S,4R)-N-((S)-(5-cyclopropyl-6- fluoropyridin-2-yl)(phenyl)methyl)- 1-(2-(5-(difluoromethyl)-1H- tetrazol-1-yl)acetyl)-4- fluoropyrrolidine-2-carboxamide |
| GZ-294 | 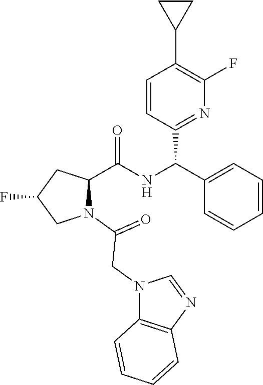 [SMILES: O=C(N[C@@H](c1ccccc1)c1ccc(C2CC2)c(F)n1)[C@@H]1C[C@@H](F)CN1C(=O)Cn1cnc2ccccc21] | (2S,4R)-1-[2-(1H-1,3-benzodiazol- 1-yl)acetyl]-N-[(S)-(5-cyclopropyl- 6-fluoropyridin-2- yl)(phenyl)methyl]-4- fluoropyrrolidine-2-carboxamide |
| GZ-295 | 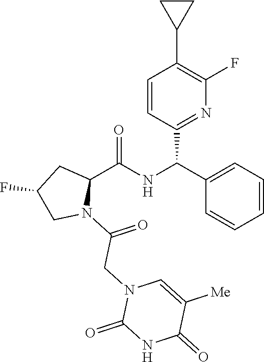 [SMILES: Cc1cn(CC(=O)N2C[C@H](F)C[C@H]2C(=O)N[C@@H](c2ccccc2)c2ccc(C3CC3)c(F)n2)c(=O)[nH]c1=O] | (2S,4R)-N-[(S)-(5-cyclopropyl-6- fluoropyridin-2-yl)(phenyl)methyl]- 4-fluoro-1-[2-(5-methyl-2,4-dioxo- 1,2,3,4-tetrahydropyrimidin-1- yl)acetyl]pyrrolidine-2- carboxamide |
| GZ-296 | 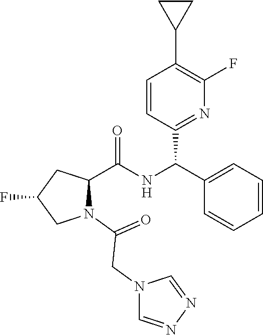 [SMILES: O=C(N[C@@H](c1ccccc1)c1ccc(C2CC2)c(F)n1)[C@@H]1C[C@@H](F)CN1C(=O)Cn1cnnc1] | (2S,4R)-N-[(S)-(5-cyclopropyl-6- fluoropyridin-2-yl)(phenyl)methyl]- 4-fluoro-1-[2-(4H-1,2,4-triazol-4- yl)acetyl]pyrrolidine-2- carboxamide |
| GZ-297 | 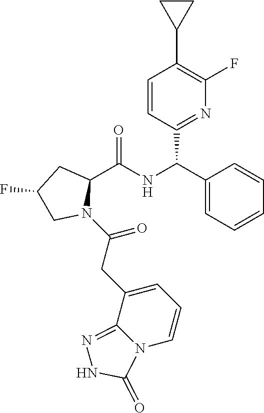 [SMILES: O=C(N[C@@H](c1ccccc1)c1ccc(C2CC2)c(F)n1)[C@@H]1C[C@@H](F)CN1C(=O)Cc1cccn2c(=O)[nH]nc12] | (2S,4R)-N-[(S)-(5-cyclopropyl-6- fluoropyridin-2-yl)(phenyl)methyl]- 4-fluoro-1-(2-{3-oxo-2H,3H- [1,2,4]triazolo[4,3-a]pyridin-8- yl}acetyl)pyrrolidine-2- carboxamide |
| GZ-298 | 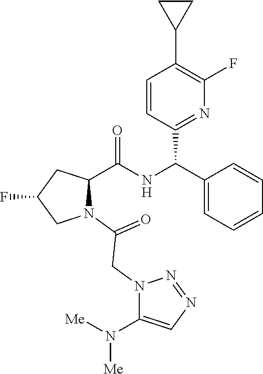 [SMILES: CN(C)c1cnnn1CC(=O)N1C[C@H](F)C[C@H]1C(=O)N[C@@H](c1ccccc1)c1ccc(C2CC2)c(F)n1] | (2S,4R)-N-[(S)-(5-cyclopropyl-6- fluoropyridin-2-yl)(phenyl)methyl]- 1-{2-[5-(dimethylamino)-1H-1,2,3- triazol-1-yl]acetyl}-4- fluoropyrrolidine-2-carboxamide |
| GZ-299 | 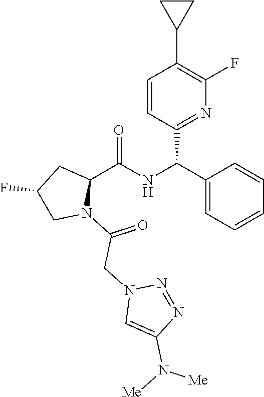 [SMILES: CN(C)c1cn(CC(=O)N2C[C@H](F)C[C@H]2C(=O)N[C@@H](c2ccccc2)c2ccc(C3CC3)c(F)n2)nn1] | (2S,4R)-N-[(S)-(5-cyclopropyl-6- fluoropyridin-2-yl)(phenyl)methyl]- 1-{2-[4-(dimethylamino)-1H-1,2,3- triazol-1-yl]acetyl}-4- fluoropyrrolidine-2-carboxamide |
| GZ-300 | 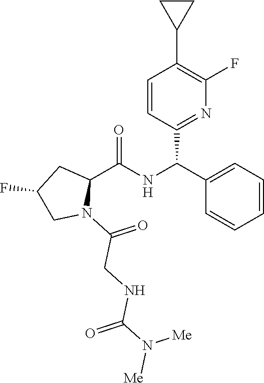 [SMILES: CN(C)C(=O)NCC(=O)N1C[C@H](F)C[C@H]1C(=O)N[C@@H](c1ccccc1)c1ccc(C2CC2)c(F)n1] | (2S,4R)-N-[(S)-(5-cyclopropyl-6- fluoropyridin-2-yl)(phenyl)methyl]- 1-{2- [(dimethylcarbamoyl)amino]acetyl}- 4-fluoropyrrolidine-2-carboxamide |
| GZ-301 | 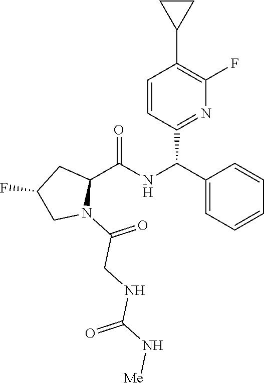 [SMILES: CNC(=O)NCC(=O)N1C[C@H](F)C[C@H]1C(=O)N[C@@H](c1ccccc1)c1ccc(C2CC2)c(F)n1] | (2S,4R)-N-[(S)-(5-cyclopropyl-6- fluoropyridin-2-yl)(phenyl)methyl]- 4-fluoro-1-{2- [(methylcarbamoyl)amino]acetyl} pyrrolidine-2-carboxamide |
| GZ-302 | 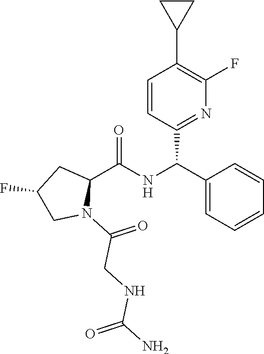 [SMILES: NC(=O)NCC(=O)N1C[C@H](F)C[C@H]1C(=O)N[C@@H](c1ccccc1)c1ccc(C2CC2)c(F)n1] | (2S,4R)-1-[2- (carbamoylamino)acetyl]-N-[(S)- (5-cyclopropyl-6-fluoropyridin-2- yl)(phenyl)methyl]-4- fluoropyrrolidine-2-carboxamide |
| GZ-303 | 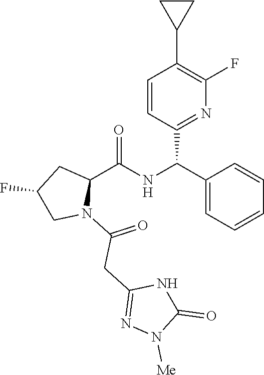 [SMILES: Cn1nc(CC(=O)N2C[C@H](F)C[C@H]2C(=O)N[C@@H](c2ccccc2)c2ccc(C3CC3)c(F)n2)[nH]c1=O] | (2S,4R)-N-[(S)-(5-cyclopropyl-6- fluoropyridin-2-yl)(phenyl)methyl]- 4-fluoro-1-[2-(1-methyl-5-oxo-4,5- dihydro-1H-1,2,4-triazol-3- yl)acetyl]pyrrolidine-2- carboxamide |
| GZ-304 | 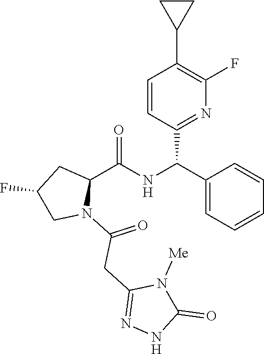 [SMILES: Cn1c(CC(=O)N2C[C@H](F)C[C@H]2C(=O)N[C@@H](c2ccccc2)c2ccc(C3CC3)c(F)n2)n[nH]c1=O] | (2S,4R)-N-[(S)-(5-cyclopropyl-6- fluoropyridin-2-yl)(phenyl)methyl]- 4-fluoro-1-[2-(4-methyl-5-oxo-4,5- dihydro-1H-1,2,4-triazol-3- yl)acetyl]pyrrolidine-2- carboxamide |
| GZ-305 | 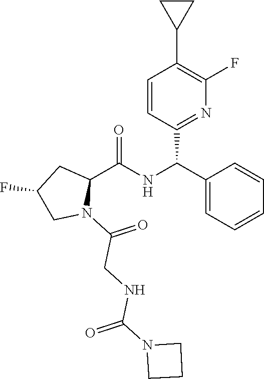 [SMILES: O=C(N[C@@H](c1ccccc1)c1ccc(C2CC2)c(F)n1)[C@@H]1C[C@@H](F)CN1C(=O)CNC(=O)N1CCC1] | (2S,4R)-1-{2-[(azetidine-1- carbonyl)amino]acetyl}-N-[(S)-(5- cyclopropyl-6-fluoropyridin-2- yl)(phenyl)methyl]-4- fluoropyrrolidine-2-carboxamide |
| GZ-306 | 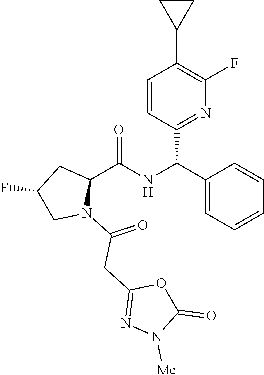 [SMILES: Cn1nc(CC(=O)N2C[C@H](F)C[C@H]2C(=O)N[C@@H](c2ccccc2)c2ccc(C3CC3)c(F)n2)oc1=O] | (2S,4R)-N-[(S)-(5-cyclopropyl-6- fluoropyridin-2-yl)(phenyl)methyl]- 4-fluoro-1-[2-(4-methyl-5-oxo-4,5- dihydro-1,3,4-oxadiazol-2- yl)acetyl]pyrrolidine-2- carboxamide |
| GZ-307 | 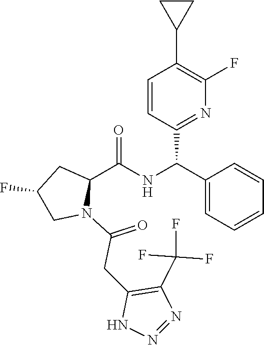 [SMILES: O=C(N[C@@H](c1ccccc1)c1ccc(C2CC2)c(F)n1)[C@@H]1C[C@@H](F)CN1C(=O)Cc1[nH]nnc1C(F)(F)F] | (2S,4R)-N-[(S)-(5-cyclopropyl-6- fluoropyridin-2-yl)(phenyl)methyl]- 4-fluoro-1-{2-[4-(trifluoromethyl)- 1H-1,2,3-triazol-5- yl]acetyl} pyrrolidine-2- carboxamide |
| GZ-308 | 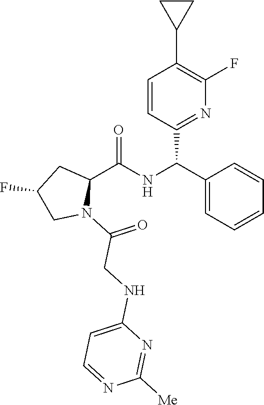 [SMILES: Cc1nccc(NCC(=O)N2C[C@H](F)C[C@H]2C(=O)N[C@@H](c2ccccc2)c2ccc(C3CC3)c(F)n2)n1] | (2S,4R)-N-[(S)-(5-cyclopropyl-6- fluoropyridin-2-yl)(phenyl)methyl]- 4-fluoro-1-{2-[(2-methylpyrimidin- 4-yl)amino]acetyl } pyrrolidine-2- carboxamide |
| GZ-309 | 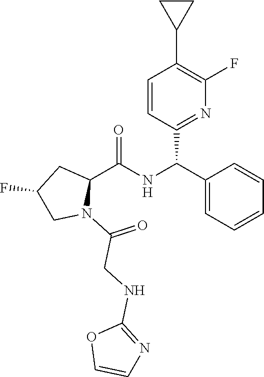 [SMILES: O=C(N[C@@H](c1ccccc1)c1ccc(C2CC2)c(F)n1)[C@@H]1C[C@@H](F)CN1C(=O)CNc1ncco1] | (2S,4R)-N-[(S)-(5-cyclopropyl-6- fluoropyridin-2-yl)(phenyl)methyl]- 4-fluoro-1-{2-[(1,3-oxazol-2- yl)amino]acetyl} pyrrolidine-2- carboxamide |
| GZ-310 | 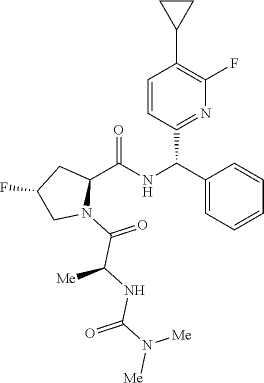 [SMILES: C[C@H](NC(=O)N(C)C)C(=O)N1C[C@H](F)C[C@H]1C(=O)N[C@@H](c1ccccc1)c1ccc(C2CC2)c(F)n1] | (2S,4R)-N-[(S)-(5-cyclopropyl-6- fluoropyridin-2-yl)(phenyl)methyl]- 1-[(2S)-2- [(dimethylcarbamoyl)amino] propanoyl]-4-fluoropyrrolidine-2- carboxamide |
| GZ-311 | 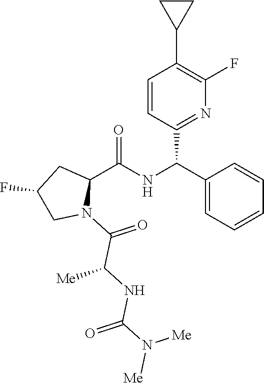 [SMILES: C[C@@H](NC(=O)N(C)C)C(=O)N1C[C@H](F)C[C@H]1C(=O)N[C@@H](c1ccccc1)c1ccc(C2CC2)c(F)n1] | (2S,4R)-N-[(S)-(5-cyclopropyl-6- fluoropyridin-2-yl)(phenyl)methyl]- 1-[(2R)-2- [(dimethylcarbamoyl)amino] propanoyl]-4-fluoropyrrolidine-2- carboxamide |
| GZ-312 | 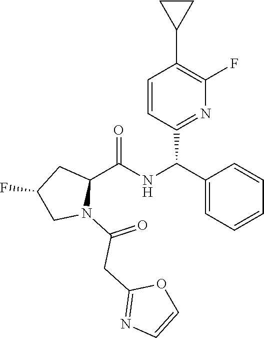 [SMILES: O=C(N[C@@H](c1ccccc1)c1ccc(C2CC2)c(F)n1)[C@@H]1C[C@@H](F)CN1C(=O)Cc1ncco1] | (2S,4R)-N-[(S)-(5-cyclopropyl-6- fluoropyridin-2-yl)(phenyl)methyl]- 4-fluoro-1-[2-(1,3-oxazol-2- yl)acetyl]pyrrolidine-2- carboxamide |
| GZ-313 | 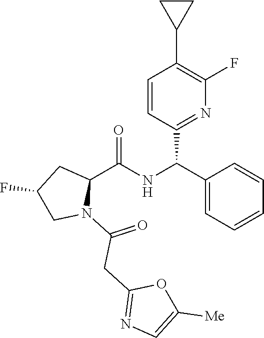 [SMILES: Cc1cnc(CC(=O)N2C[C@H](F)C[C@H]2C(=O)N[C@@H](c2ccccc2)c2ccc(C3CC3)c(F)n2)o1] | (2S,4R)-N-[(S)-(5-cyclopropyl-6- fluoropyridin-2-yl)(phenyl)methyl]- 4-fluoro-1-[2-(5-methyl-1,3-oxazol- 2-yl)acetyl]pyrrolidine-2- carboxamide |
| GZ-314 | 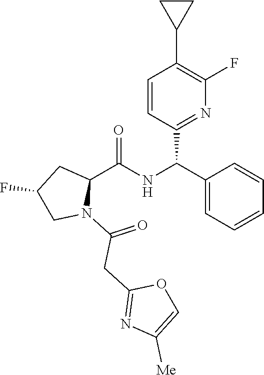 [SMILES: Cc1coc(CC(=O)N2C[C@H](F)C[C@H]2C(=O)N[C@@H](c2ccccc2)c2ccc(C3CC3)c(F)n2)n1] | (2S,4R)-N-[(S)-(5-cyclopropyl-6- fluoropyridin-2-yl)(phenyl)methyl]- 4-fluoro-1-[2-(4-methyl-1,3-oxazol- 2-yl)acetyl]pyrrolidine-2- carboxamide |
| GZ-315 | 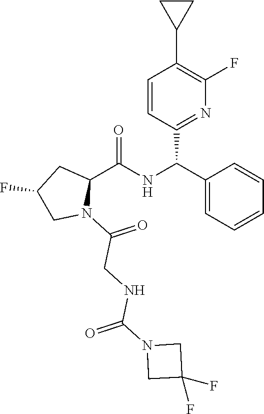 [SMILES: O=C(N[C@@H](c1ccccc1)c1ccc(C2CC2)c(F)n1)[C@@H]1C[C@@H](F)CN1C(=O)CNC(=O)N1CC(F)(F)C1] | (2S,4R)-N-[(S)-(5-cyclopropyl-6- fluoropyridin-2-yl)(phenyl)methyl]- 1-{2-[(3,3-difluoroazetidine-1- carbonyl)amino]acetyl}-4- fluoropyrrolidine-2-carboxamide |
| GZ-316 | 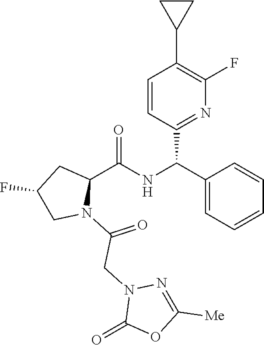 [SMILES: Cc1nn(CC(=O)N2C[C@H](F)C[C@H]2C(=O)N[C@@H](c2ccccc2)c2ccc(C3CC3)c(F)n2)c(=O)o1] | (2S,4R)-N-[(S)-(5-cyclopropyl-6- fluoropyridin-2-yl)(phenyl)methyl]- 4-fluoro-1-[2-(5-methyl-2-oxo-2,3- dihydro-1,3,4-oxadiazol-3- yl)acetyl]pyrrolidine-2- carboxamide |
| GZ-317 | 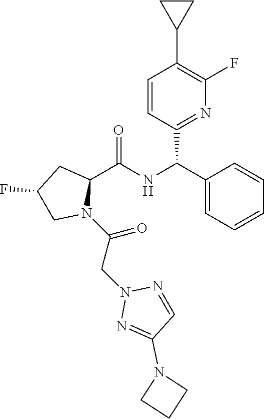 [SMILES: O=C(N[C@@H](c1ccccc1)c1ccc(C2CC2)c(F)n1)[C@@H]1C[C@@H](F)CN1C(=O)Cn1ncc(N2CCC2)n1] | (2S,4R)-1-{2-[4-(azetidin-1-yl)-2H- 1,2,3-triazol-2-yl]acetyl}-N-[(S)-(5- cyclopropyl-6-fluoropyridin-2- yl)(phenyl)methyl]-4- fluoropyrrolidine-2-carboxamide |
| GZ-318 | 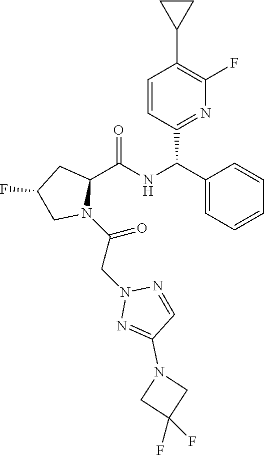 [SMILES: O=C(N[C@@H](c1ccccc1)c1ccc(C2CC2)c(F)n1)[C@@H]1C[C@@H](F)CN1C(=O)Cn1ncc(N2CC(F)(F)C2)n1] | (2S,4R)-N-[(S)-(5-cyclopropyl-6- fluoropyridin-2-yl)(phenyl)methyl]- 1-{2-[4-(3,3-difluoroazetidin-1-yl)- 2H-1,2,3-triazol-2-yl]acetyl}-4- fluoropyrrolidine-2-carboxamide |
| GZ-319 | 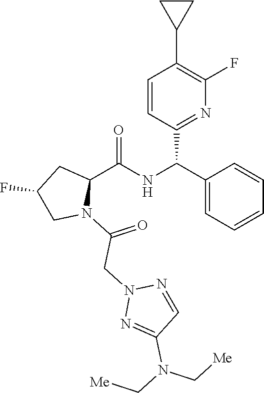 [SMILES: CCN(CC)c1cnn(CC(=O)N2C[C@H](F)C[C@H]2C(=O)N[C@@H](c2ccccc2)c2ccc(C3CC3)c(F)n2)n1] | (2S,4R)-N-[(S)-(5-cyclopropyl-6- fluoropyridin-2-yl)(phenyl)methyl]- 1-{2-[4-(diethylamino)-2H-1,2,3- triazol-2-yl]acetyl}-4- fluoropyrrolidine-2-carboxamide |
| GZ-320 | 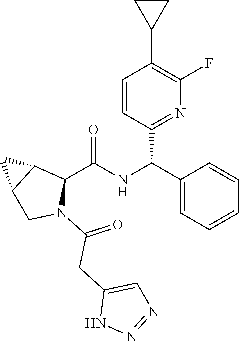 [SMILES: O=C(N[C@@H](c1ccccc1)c1ccc(C2CC2)c(F)n1)[C@@H]1[C@H]2C[C@H]2CN1C(=O)Cc1cnn[nH]1] | (1S,2S,5R)-N-[(S)-(5-cyclopropyl- 6-fluoropyridin-2- yl)(phenyl)methyl]-3-[2-(1H-1,2,3- triazol-5-yl)acetyl]-3- azabicyclo[3.1.0]hexane-2- carboxamide |
| GZ-321 | 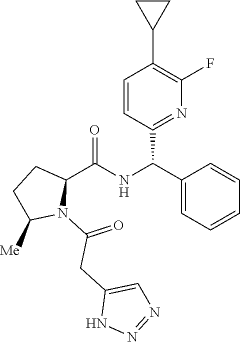 [SMILES: C[C@H]1CC[C@@H](C(=O)N[C@@H](c2ccccc2)c2ccc(C3CC3)c(F)n2)N1C(=O)Cc1cnn[nH]1] | (2S,5S)-N-[(S)-(5-cyclopropyl-6- fluoropyridin-2-yl)(phenyl)methyl]- 5-methyl-1-[2-(1H-1,2,3-triazol-5- yl)acetyl]pyrrolidine-2- carboxamide |
| GZ-322 | 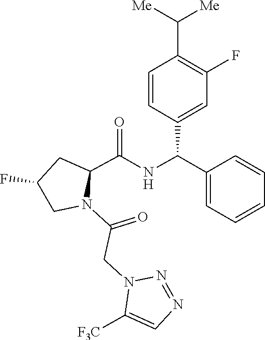 [SMILES: CC(C)c1ccc([C@@H](NC(=O)[C@@H]2C[C@@H](F)CN2C(=O)Cn2nncc2C(F)(F)F)c2ccccc2)cc1F] | (2S,4R)-4-fluoro-N-((S)-(3-fluoro- 4- isopropylphenyl)(phenyl)methyl)- 1-(2-(5-(trifluoromethyl)-1H-1,2,3- triazol-1-yl)acetyl)pyrrolidine-2- carboxamide |
| GZ-323 | 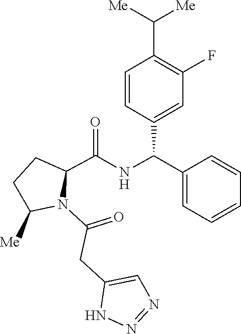 [SMILES: CC(C)c1ccc([C@@H](NC(=O)[C@@H]2CC[C@H](C)N2C(=O)Cc2cnn[nH]2)c2ccccc2)cc1F] | (2S,5S)-N-[(S)-[3-fluoro-4- (propan-2- yl)phenyl](phenyl)methyl]-5- methyl-1-[2-(1H-1,2,3-triazol-5- yl)acetyl]pyrrolidine-2- carboxamide |
| GZ-324 | 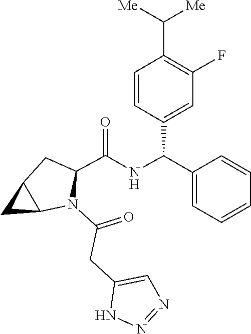 [SMILES: CC(C)c1ccc([C@@H](NC(=O)[C@@H]2C[C@@H]3C[C@@H]3N2C(=O)Cc2cnn[nH]2)c2ccccc2)cc1F] | (1S,3S,5S)-N-[(S)-[3-fluoro-4- (propan-2- yl)phenyl](phenyl)methyl]-2-[2- (1H-1,2,3-triazol-5-yl)acetyl]-2- azabicyclo[3.1.0]hexane-3- carboxamide |
| GZ-325 | 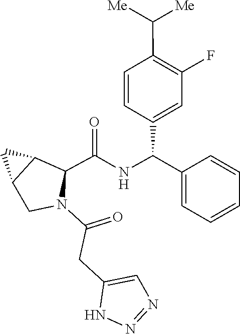 [SMILES: CC(C)c1ccc([C@@H](NC(=O)[C@@H]2[C@H]3C[C@H]3CN2C(=O)Cc2cnn[nH]2)c2ccccc2)cc1F] | (1S,2S,5R)-N-[(S)-[3-fluoro-4- (propan-2- yl)phenyl](phenyl)methyl]-3-[2- (1H-1,2,3-triazol-5-yl)acetyl]-3- azabicyclo[3.1.0]hexane-2- carboxamide |
| GZ-326 | 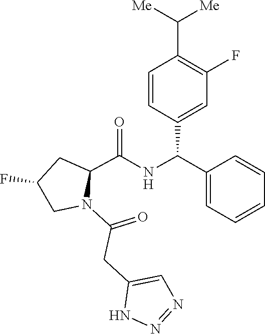 [SMILES: CC(C)c1ccc([C@@H](NC(=O)[C@@H]2C[C@@H](F)CN2C(=O)Cc2cnn[nH]2)c2ccccc2)cc1F] | (2S,4R)-4-fluoro-N-[(S)-[3-fluoro- 4-(propan-2- yl)phenyl](phenyl)methyl]-1-[2- (1H-1,2,3-triazol-5- yl)acetyl]pyrrolidine-2- carboxamide |
| GZ-327 | 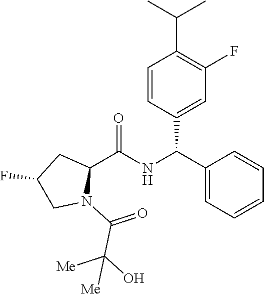 [SMILES: CC(C)c1ccc([C@@H](NC(=O)[C@@H]2C[C@@H](F)CN2C(=O)C(C)(C)O)c2ccccc2)cc1F] | (2S,4R)-4-fluoro-N-[(S)-[3-fluoro- 4-(propan-2- yl)phenyl](phenyl)methyl]-1-(2- hydroxy-2- methylpropanoyl)pyrrolidine-2- carboxamide |
| GZ-328 | 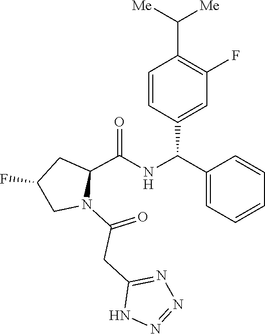 [SMILES: CC(C)c1ccc([C@@H](NC(=O)[C@@H]2C[C@@H](F)CN2C(=O)Cc2nnn[nH]2)c2ccccc2)cc1F] | (2S,4R)-4-fluoro-N-[(S)-[3-fluoro- 4-(propan-2- yl)phenyl](phenyl)methyl]-1-[2- (1H-1,2,3,4-tetrazol-5- yl)acetyl]pyrrolidine-2- carboxamide |
| GZ-329 | 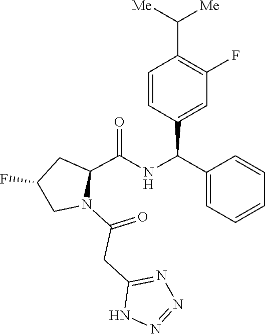 [SMILES: CC(C)c1ccc([C@H](NC(=O)[C@@H]2C[C@@H](F)CN2C(=O)Cc2nnn[nH]2)c2ccccc2)cc1F] | (2S,4R)-4-fluoro-N-[(R)-[3-fluoro- 4-(propan-2- yl)phenyl](phenyl)methyl]-1-[2- (1H-1,2,3,4-tetrazol-5- yl)acetyl]pyrrolidine-2- carboxamide |
| GZ-330 | 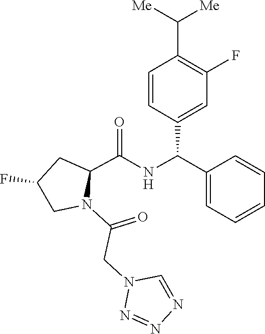 [SMILES: CC(C)c1ccc([C@@H](NC(=O)[C@@H]2C[C@@H](F)CN2C(=O)Cn2cnnn2)c2ccccc2)cc1F] | (2S,4R)-4-fluoro-N-[(S)-[3-fluoro- 4-(propan-2- yl)phenyl](phenyl)methyl]-1-[2- (1H-1,2,3,4-tetrazol-1- yl)acetyl]pyrrolidine-2- carboxamide |
| GZ-331 | 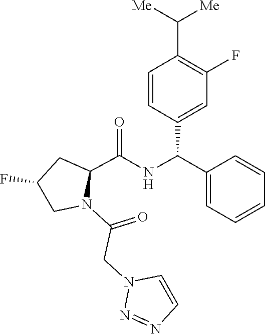 [SMILES: CC(C)c1ccc([C@@H](NC(=O)[C@@H]2C[C@@H](F)CN2C(=O)Cn2ccnn2)c2ccccc2)cc1F] | (2S,4R)-4-fluoro-N-[(S)-[3-fluoro- 4-(propan-2- yl)phenyl](phenyl)methyl]-1-[2- (1H-1,2,3-triazol-1- yl)acetyl]pyrrolidine-2- carboxamide |
| GZ-332 | 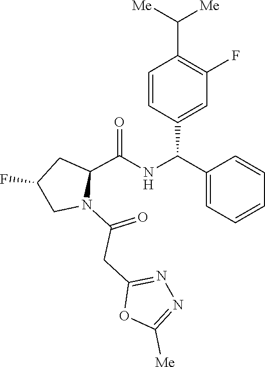 [SMILES: Cc1nnc(CC(=O)N2C[C@H](F)C[C@H]2C(=O)N[C@@H](c2ccccc2)c2ccc(C(C)C)c(F)c2)o1] | (2S,4R)-4-fluoro-N-[(S)-[3-fluoro- 4-(propan-2- yl)phenyl](phenyl)methyl]-1-[2-(5- methyl-1,3,4-oxadiazol-2- yl)acetyl]pyrrolidine-2- carboxamide |
| GZ-333 | 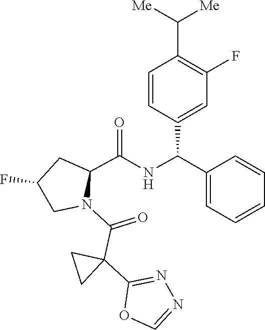 [SMILES: CC(C)c1ccc([C@@H](NC(=O)[C@@H]2C[C@@H](F)CN2C(=O)C2(c3nnco3)CC2)c2ccccc2)cc1F] | (2S,4R)-4-fluoro-N-[(S)-[3-fluoro- 4-(propan-2- yl)phenyl](phenyl)methyl]-1-[1- (1,3,4-oxadiazol-2- yl)cyclopropanecarbonyl]pyrrolidin e-2-carboxamide |
| GZ-334 | 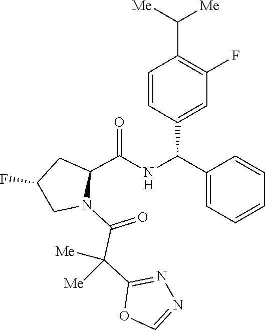 [SMILES: CC(C)c1ccc([C@@H](NC(=O)[C@@H]2C[C@@H](F)CN2C(=O)C(C)(C)c2nnco2)c2ccccc2)cc1F] | (2S,4R)-4-fluoro-N-[(S)-[3-fluoro- 4-(propan-2- yl)phenyl](phenyl)methyl]-1-[2- methyl-2-(1,3,4-oxadiazol-2- yl)propanoyl]pyrrolidine-2- carboxamide |
| GZ-335 | 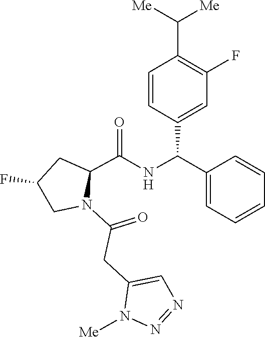 [SMILES: CC(C)c1ccc([C@@H](NC(=O)[C@@H]2C[C@@H](F)CN2C(=O)Cc2cnnn2C)c2ccccc2)cc1F] | (2S,4R)-4-fluoro-N-[(S)-[3-fluoro- 4-(propan-2- yl)phenyl](phenyl)methyl]-1-[2-(1- methyl-1H-1,2,3-triazol-5- yl)acetyl]pyrrolidine-2- carboxamide |
| GZ-336 | 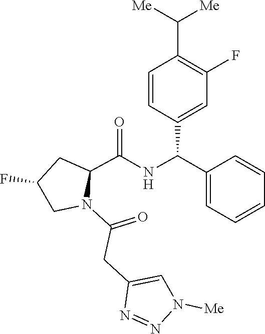 [SMILES: CC(C)c1ccc([C@@H](NC(=O)[C@@H]2C[C@@H](F)CN2C(=O)Cc2cn(C)nn2)c2ccccc2)cc1F] | (2S,4R)-4-fluoro-N-[(S)-[3-fluoro- 4-(propan-2- yl)phenyl](phenyl)methyl]-1-[2-(1- methyl-1H-1,2,3-triazol-4- yl)acetyl]pyrrolidine-2- carboxamide |
| GZ-337 | 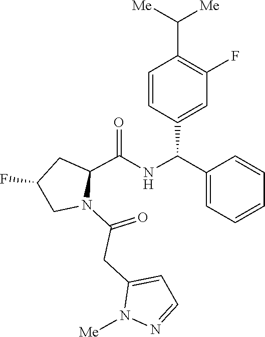 [SMILES: CC(C)c1ccc([C@@H](NC(=O)[C@@H]2C[C@@H](F)CN2C(=O)Cc2ccnn2C)c2ccccc2)cc1F] | (2S,4R)-4-fluoro-N-[(S)-[3-fluoro- 4-(propan-2- yl)phenyl](phenyl)methyl]-1-[2-(1- methyl-1H-pyrazol-5- yl)acetyl]pyrrolidine-2- carboxamide |
| GZ-338 | 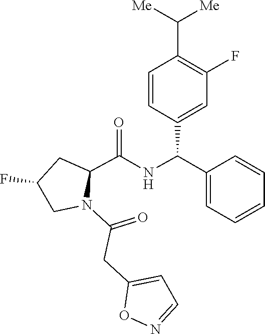 [SMILES: CC(C)c1ccc([C@@H](NC(=O)[C@@H]2C[C@@H](F)CN2C(=O)Cc2ccno2)c2ccccc2)cc1F] | (2S,4R)-4-fluoro-N-[(S)-[3-fluoro- 4-(propan-2- yl)phenyl](phenyl)methyl]-1-[2- (1,2-oxazol-5-yl)acetyl]pyrrolidine- 2-carboxamide |
| GZ-339 | 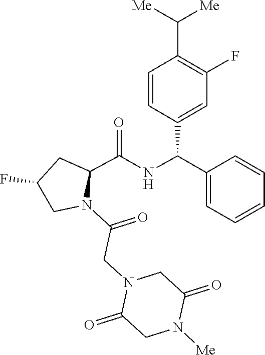 [SMILES: CC(C)c1ccc([C@@H](NC(=O)[C@@H]2C[C@@H](F)CN2C(=O)CN2CC(=O)N(C)CC2=O)c2ccccc2)cc1F] | (2S,4R)-4-fluoro-N-[(S)-[3-fluoro- 4-(propan-2- yl)phenyl](phenyl)methyl]-1-[2-(4- methyl-2,5-dioxopiperazin-1- yl)acetyl]pyrrolidine-2- carboxamide |
| GZ-340 | 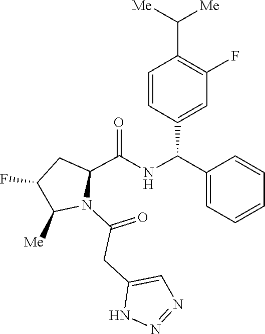 [SMILES: CC(C)c1ccc([C@@H](NC(=O)[C@@H]2C[C@@H](F)[C@H](C)N2C(=O)Cc2cnn[nH]2)c2ccccc2)cc1F] | (2S,4R,5S)-4-fluoro-N-[(S)-[3- fluoro-4-(propan-2- yl)phenyl](phenyl)methyl]-5- methyl-1-[2-(1H-1,2,3-triazol-5- yl)acetyl]pyrrolidine-2- carboxamide |
| GZ-341 | 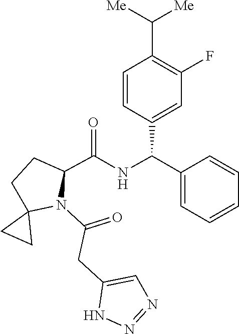 [SMILES: CC(C)c1ccc([C@@H](NC(=O)[C@@H]2CCC3(CC3)N2C(=O)Cc2cnn[nH]2)c2ccccc2)cc1F] | (5S)-N-[(S)-[3-fluoro-4-(propan-2- yl)phenyl](phenyl)methyl]-4-[2- (1H-1,2,3-triazol-5-yl)acetyl]-4- azaspiro[2.4]heptane-5- carboxamide |
| GZ-342 | 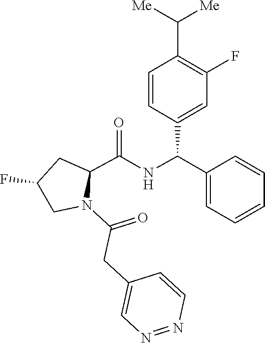 [SMILES: CC(C)c1ccc([C@@H](NC(=O)[C@@H]2C[C@@H](F)CN2C(=O)Cc2ccnnc2)c2ccccc2)cc1F] | (2S,4R)-4-fluoro-N-[(S)-[3-fluoro- 4-(propan-2- yl)phenyl](phenyl)methyl]-1-[2- (pyridazin-4-yl)acetyl]pyrrolidine- 2-carboxamide |
| GZ-343 | 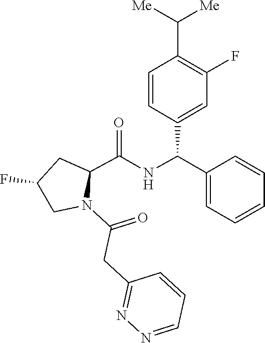 [SMILES: CC(C)c1ccc([C@@H](NC(=O)[C@@H]2C[C@@H](F)CN2C(=O)Cc2cccnn2)c2ccccc2)cc1F] | (2S,4R)-4-fluoro-N-[(S)-[3-fluoro- 4-(propan-2- yl)phenyl](phenyl)methyl]-1-[2- (pyridazin-3-yl)acetyl]pyrrolidine- 2-carboxamide |
| GZ-344 | 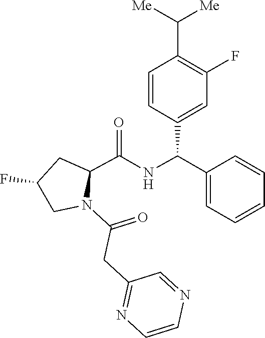 [SMILES: CC(C)c1ccc([C@@H](NC(=O)[C@@H]2C[C@@H](F)CN2C(=O)Cc2cnccn2)c2ccccc2)cc1F] | (2S,4R)-4-fluoro-N-[(S)-[3-fluoro- 4-(propan-2- yl)phenyl](phenyl)methyl]-1-[2- (pyrazin-2-yl)acetyl]pyrrolidine-2- carboxamide |
| GZ-345 | 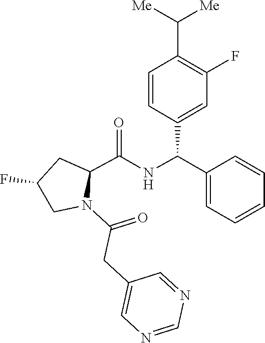 [SMILES: CC(C)c1ccc([C@@H](NC(=O)[C@@H]2C[C@@H](F)CN2C(=O)Cc2cncnc2)c2ccccc2)cc1F] | (2S,4R)-4-fluoro-N-[(S)-[3-fluoro- 4-(propan-2- yl)phenyl](phenyl)methyl]-1-[2- (pyrimidin-5-yl)acetyl]pyrrolidine- 2-carboxamide |
| GZ-346 | 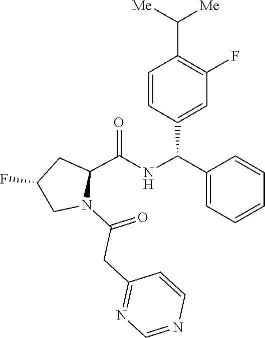 [SMILES: CC(C)c1ccc([C@@H](NC(=O)[C@@H]2C[C@@H](F)CN2C(=O)Cc2ccncn2)c2ccccc2)cc1F] | (2S,4R)-4-fluoro-N-[(S)-[3-fluoro- 4-(propan-2- yl)phenyl](phenyl)methyl]-1-[2- (pyrimidin-4-yl)acetyl]pyrrolidine- 2-carboxamide |
| GZ-347 | 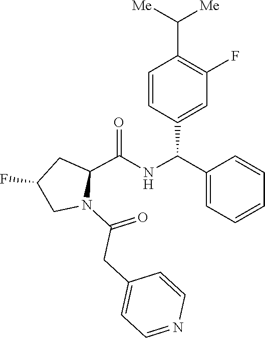 [SMILES: CC(C)c1ccc([C@@H](NC(=O)[C@@H]2C[C@@H](F)CN2C(=O)Cc2ccncc2)c2ccccc2)cc1F] | (2S,4R)-4-fluoro-N-[(S)-[3-fluoro- 4-(propan-2- yl)phenyl](phenyl)methyl]-1-[2- (pyridin-4-yl)acetyl]pyrrolidine-2- carboxamide |
| GZ-348 | 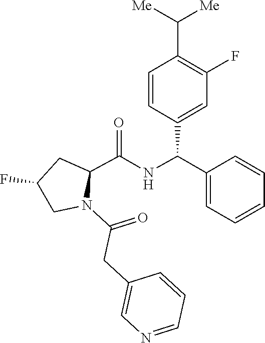 [SMILES: CC(C)c1ccc([C@@H](NC(=O)[C@@H]2C[C@@H](F)CN2C(=O)Cc2cccnc2)c2ccccc2)cc1F] | (2S,4R)-4-fluoro-N-[(S)-[3-fluoro- 4-(propan-2- yl)phenyl](phenyl)methyl]-1-[2- (pyridin-3-yl)acetyl]pyrrolidine-2- carboxamide |
| GZ-349 | 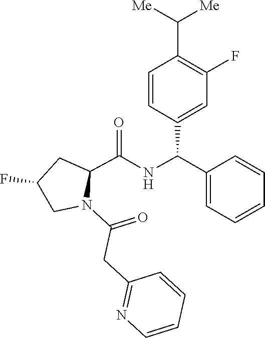 [SMILES: CC(C)c1ccc([C@@H](NC(=O)[C@@H]2C[C@@H](F)CN2C(=O)Cc2ccccn2)c2ccccc2)cc1F] | (2S,4R)-4-fluoro-N-[(S)-[3-fluoro- 4-(propan-2- yl)phenyl](phenyl)methyl]-1-[2- (pyridin-2-yl)acetyl]pyrrolidine-2- carboxamide |
| GZ-350 | 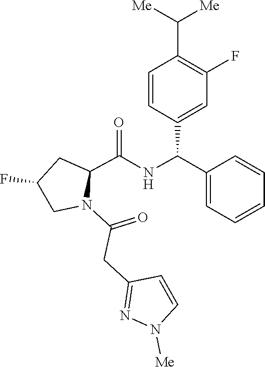 [SMILES: CC(C)c1ccc([C@@H](NC(=O)[C@@H]2C[C@@H](F)CN2C(=O)Cc2ccn(C)n2)c2ccccc2)cc1F] | (2S,4R)-4-fluoro-N-[(S)-[3-fluoro- 4-(propan-2- yl)phenyl](phenyl)methyl]-1-[2-(1- methyl-1H-pyrazol-3- yl)acetyl]pyrrolidine-2- carboxamide |
| GZ-351 | 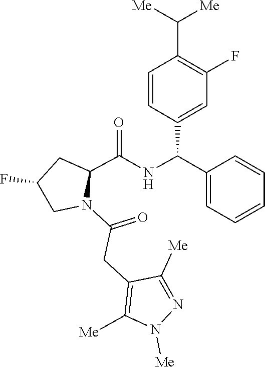 [SMILES: Cc1nn(C)c(C)c1CC(=O)N1C[C@H](F)C[C@H]1C(=O)N[C@@H](c1ccccc1)c1ccc(C(C)C)c(F)c1] | (2S,4R)-4-fluoro-N-[(S)-[3-fluoro- 4-(propan-2- yl)phenyl](phenyl)methyl]-1-[2- (1,3,5-trimethyl-1H-pyrazol-4- yl)acetyl]pyrrolidine-2- carboxamide |
| GZ-352 | 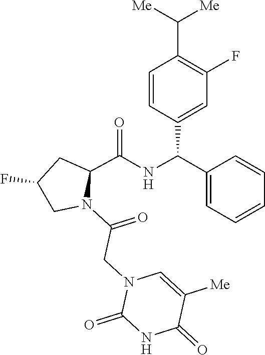 [SMILES: Cc1cn(CC(=O)N2C[C@H](F)C[C@H]2C(=O)N[C@@H](c2ccccc2)c2ccc(C(C)C)c(F)c2)c(=O)[nH]c1=O] | (2S,4R)-4-fluoro-N-[(S)-[3-fluoro- 4-(propan-2- yl)phenyl](phenyl)methyl]-1-[2-(5- methyl-2,4-dioxo-1,2,3,4- tetrahydropyrimidin-1- yl)acetyl]pyrrolidine-2- carboxamide |
| GZ-353 | 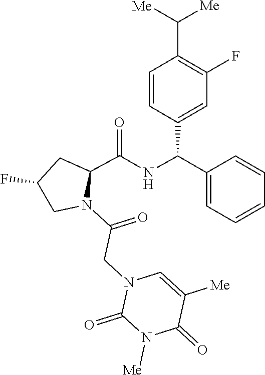 [SMILES: Cc1cn(CC(=O)N2C[C@H](F)C[C@H]2C(=O)N[C@@H](c2ccccc2)c2ccc(C(C)C)c(F)c2)c(=O)n(C)c1=O] | (2S,4R)-1-[2-(3,5-dimethyl-2,4- dioxo-1,2,3,4-tetrahydropyrimidin- 1-yl)acetyl]-4-fluoro-N-[(S)-[3- fluoro-4-(propan-2- yl)phenyl](phenyl)methyl]pyrrolidine- 2-carboxamide |
| GZ-354 | 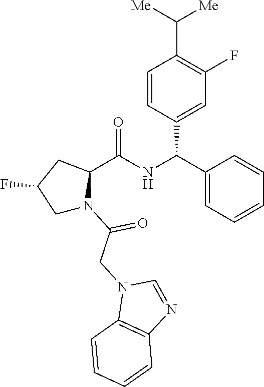 [SMILES: CC(C)c1ccc([C@@H](NC(=O)[C@@H]2C[C@@H](F)CN2C(=O)Cn2cnc3ccccc32)c2ccccc2)cc1F] | (2S,4R)-1-[2-(1H-1,3-benzodiazol- 1-yl)acetyl]-4-fluoro-N-[(S)-[3- fluoro-4-(propan-2- yl)phenyl](phenyl)methyl]pyrrolidine- 2-carboxamide |
| GZ-355 | 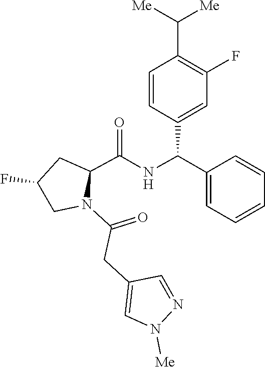 [SMILES: CC(C)c1ccc([C@@H](NC(=O)[C@@H]2C[C@@H](F)CN2C(=O)Cc2cnn(C)c2)c2ccccc2)cc1F] | (2S,4R)-4-fluoro-N-[(S)-[3-fluoro- 4-(propan-2- yl)phenyl](phenyl)methyl]-1-[2-(1- methyl-1H-pyrazol-4- yl)acetyl]pyrrolidine-2- carboxamide |
| GZ-356 | 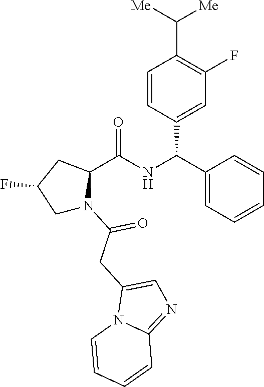 [SMILES: CC(C)c1ccc([C@@H](NC(=O)[C@@H]2C[C@@H](F)CN2C(=O)Cc2cnc3ccccn23)c2ccccc2)cc1F] | (2S,4R)-4-fluoro-N-[(S)-[3-fluoro- 4-(propan-2- yl)phenyl](phenyl)methyl]-1-(2- {imidazo[1,2-a]pyridin-3- yl}acetyl)pyrrolidine-2- carboxamide |
| GZ-357 | 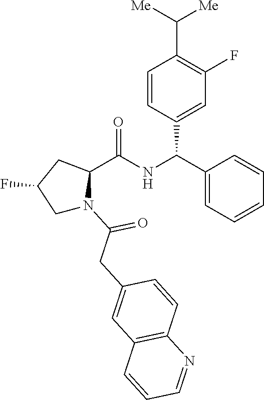 [SMILES: CC(C)c1ccc([C@@H](NC(=O)[C@@H]2C[C@@H](F)CN2C(=O)Cc2ccc3ncccc3c2)c2ccccc2)cc1F] | (2S,4R)-4-fluoro-N-[(S)-[3-fluoro- 4-(propan-2- yl)phenyl](phenyl)methyl]-1-[2- (quinolin-6-yl)acetyl]pyrrolidine-2- carboxamide |
| GZ-358 | 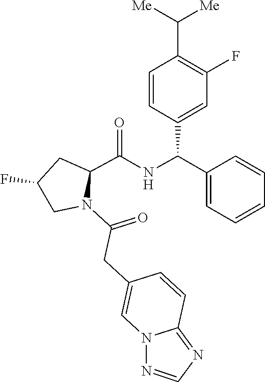 [SMILES: CC(C)c1ccc([C@@H](NC(=O)[C@@H]2C[C@@H](F)CN2C(=O)Cc2ccc3ncnn3c2)c2ccccc2)cc1F] | (2S,4R)-4-fluoro-N-[(S)-[3-fluoro- 4-(propan-2- yl)phenyl](phenyl)methyl]-1-(2- {[1,2,4]triazolo[1,5-a]pyridin-6- yl} acetyl)pyrrolidine-2- carboxamide |
| GZ-359 | 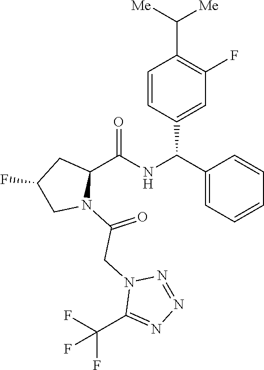 [SMILES: CC(C)c1ccc([C@@H](NC(=O)[C@@H]2C[C@@H](F)CN2C(=O)Cn2nnnc2C(F)(F)F)c2ccccc2)cc1F] | (2S,4R)-4-fluoro-N-[(S)-[3-fluoro- 4-(propan-2- yl)phenyl](phenyl)methyl]-1-{2-[5- (trifluoromethyl)-1H-1,2,3,4- tetrazol-1-yl]acetyl}pyrrolidine-2- carboxamide |
| GZ-360 | 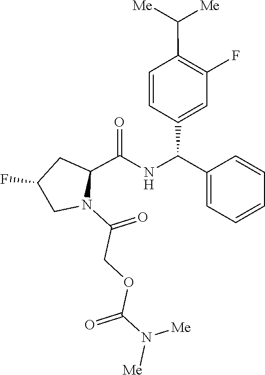 [SMILES: CC(C)c1ccc([C@@H](NC(=O)[C@@H]2C[C@@H](F)CN2C(=O)COC(=O)N(C)C)c2ccccc2)cc1F] | 2-[(2S,4R)-4-fluoro-2-{[(S)-[3- fluoro-4-(propan-2- yl)phenyl](phenyl)methyl]carbamo yl} pyrrolidin-1-yl]-2-oxoethyl N,N-dimethylcarbamate |
| GZ-361 | 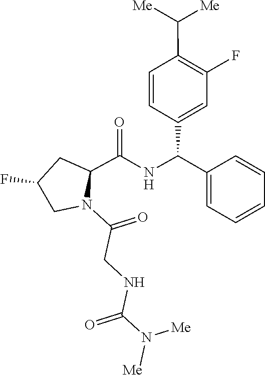 [SMILES: CC(C)c1ccc([C@@H](NC(=O)[C@@H]2C[C@@H](F)CN2C(=O)CNC(=O)N(C)C)c2ccccc2)cc1F] | (2S,4R)-1-{2- [(dimethylcarbamoyl)amino]acetyl}- 4-fluoro-N-[(S)-[3-fluoro-4- (propan-2- yl)phenyl](phenyl)methyl]pyrrolidine- 2-carboxamide |
| GZ-362 | 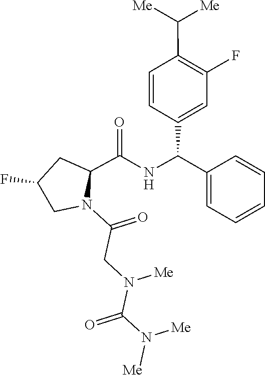 [SMILES: CC(C)c1ccc([C@@H](NC(=O)[C@@H]2C[C@@H](F)CN2C(=O)CN(C)C(=O)N(C)C)c2ccccc2)cc1F] | (2S,4R)-1-{2- [(dimethylcarbamoyl)(methyl)amino] acetyl}-4-fluoro-N-[(S)-[3- fluoro-4-(propan-2- yl)phenyl](phenyl)methyl]pyrrolidine- 2-carboxamide |
| GZ-363 | 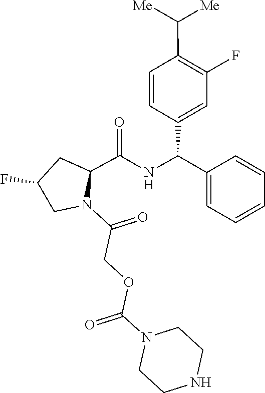 [SMILES: CC(C)c1ccc([C@@H](NC(=O)[C@@H]2C[C@@H](F)CN2C(=O)COC(=O)N2CCNCC2)c2ccccc2)cc1F] | 2-[(2S,4R)-4-fluoro-2-{[(S)-[3- fluoro-4-(propan-2- yl)phenyl](phenyl)methyl]carbamo yl} pyrrolidin-1-yl]-2-oxoethyl piperazine-1-carboxylate |
| GZ-364 | 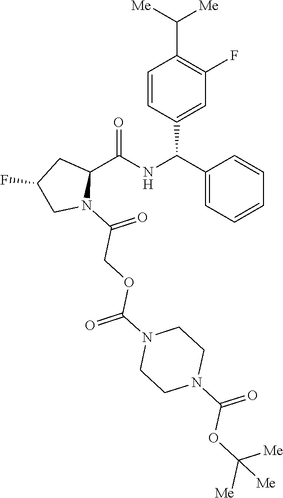 [SMILES: CC(C)c1ccc([C@@H](NC(=O)[C@@H]2C[C@@H](F)CN2C(=O)COC(=O)N2CCN(C(=O)OC(C)(C)C)CC2)c2ccccc2)cc1F] | 1-tert-butyl 4-{2-[(2S,4R)-4-fluoro- 2-{[(S)-[3-fluoro-4-(propan-2- yl)phenyl](phenyl)methyl]carbamo yl} pyrrolidin-1-yl]-2-oxoethyl } piperazine-1,4-dicarboxylate |
| GZ-365 | 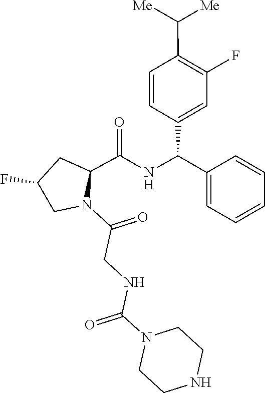 [SMILES: CC(C)c1ccc([C@@H](NC(=O)[C@@H]2C[C@@H](F)CN2C(=O)CNC(=O)N2CCNCC2)c2ccccc2)cc1F] | N-{2-[(2S,4R)-4-fluoro-2-{[(S)-[3- fluoro-4-(propan-2- yl)phenyl](phenyl)methyl]carbamo yl } pyrrolidin-1-yl]-2- oxoethyl} piperazine-1-carboxamide |
| GZ-366 | 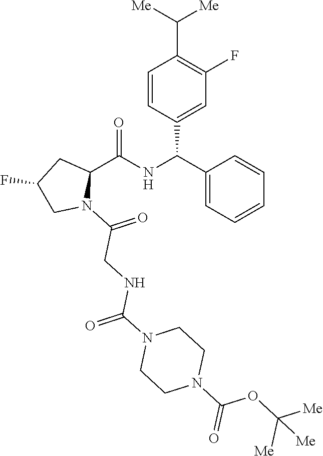 [SMILES: CC(C)c1ccc([C@@H](NC(=O)[C@@H]2C[C@@H](F)CN2C(=O)CNC(=O)N2CCN(C(=O)OC(C)(C)C)CC2)c2ccccc2)cc1F] | tert-butyl 4-({2-[(2S,4R)-4-fluoro- 2-{[(S)-[3-fluoro-4-(propan-2- yl)phenyl](phenyl)methyl]carbamo yl} pyrrolidin-1-yl]-2- oxoethyl } carbamoyl)piperazine-1- carboxylate |
| GZ-367 | 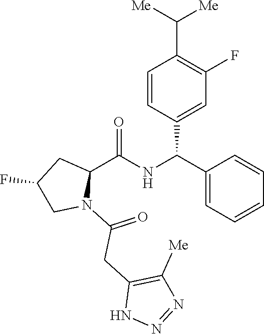 [SMILES: Cc1nn[nH]c1CC(=O)N1C[C@H](F)C[C@H]1C(=O)N[C@@H](c1ccccc1)c1ccc(C(C)C)c(F)c1] | (2S,4R)-4-fluoro-N-[(S)-[3-fluoro- 4-(propan-2- yl)phenyl](phenyl)methyl]-1-[2-(4- methyl-1H-1,2,3-triazol-5- yl)acetyl]pyrrolidine-2- carboxamide |
| GZ-368 | 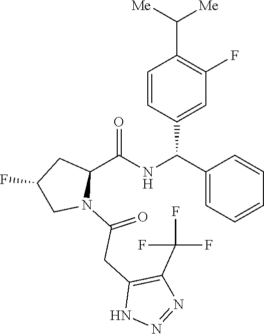 [SMILES: CC(C)c1ccc([C@@H](NC(=O)[C@@H]2C[C@@H](F)CN2C(=O)Cc2[nH]nnc2C(F)(F)F)c2ccccc2)cc1F] | (2S,4R)-4-fluoro-N-[(S)-[3-fluoro- 4-(propan-2- yl)phenyl](phenyl)methyl]-1-{2-[4- (trifluoromethyl)-1H-1,2,3-triazol- 5-yl]acetyl} pyrrolidine-2- carboxamide |
| GZ-369 | 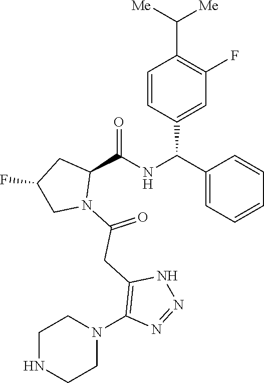 [SMILES: CC(C)c1ccc([C@@H](NC(=O)[C@@H]2C[C@@H](F)CN2C(=O)Cc2[nH]nnc2N2CCNCC2)c2ccccc2)cc1F] | (2S,4R)-4-fluoro-N-[(S)-[3-fluoro- 4-(propan-2- yl)phenyl](phenyl)methyl]-1-{2-[4- (piperazin-1-yl)-1H-1,2,3-triazol-5- yl]acetyl}pyrrolidine-2- carboxamide |
| GZ-370 | 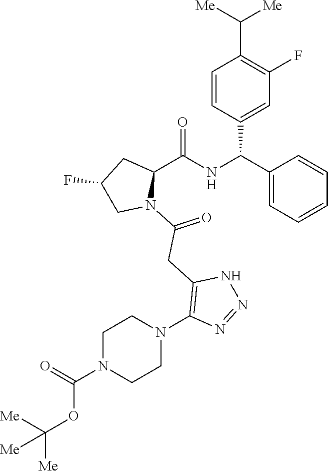 [SMILES: CC(C)c1ccc([C@@H](NC(=O)[C@@H]2C[C@@H](F)CN2C(=O)Cc2[nH]nnc2N2CCN(C(=O)OC(C)(C)C)CC2)c2ccccc2)cc1F] | tert-butyl 4-(5-{2-[(2S,4R)-4- fluoro-2-{[(S)-[3-fluoro-4-(propan- 2- yl)phenyl](phenyl)methyl]carbamo yl} pyrrolidin-1-yl]-2-oxoethyl}- 1H-1,2,3-triazol-4-yl)piperazine-1- carboxylate |
| GZ-371 | 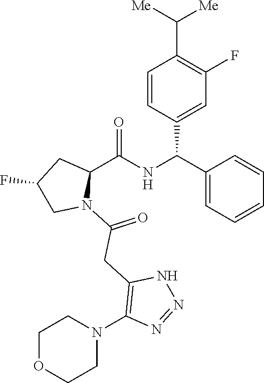 [SMILES: CC(C)c1ccc([C@@H](NC(=O)[C@@H]2C[C@@H](F)CN2C(=O)Cc2[nH]nnc2N2CCOCC2)c2ccccc2)cc1F] | (2S,4R)-4-fluoro-N-[(S)-[3-fluoro- 4-(propan-2- yl)phenyl](phenyl)methyl]-1-{2-[4- (morpholin-4-yl)-1H-1,2,3-triazol- 5-yl]acetyl} pyrrolidine-2- carboxamide |
| GZ-372 | 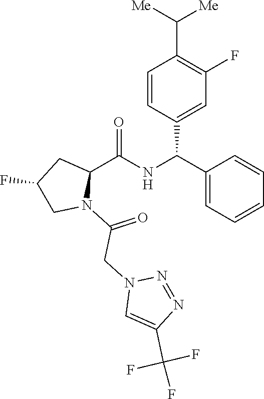 [SMILES: CC(C)c1ccc([C@@H](NC(=O)[C@@H]2C[C@@H](F)CN2C(=O)Cn2cc(C(F)(F)F)nn2)c2ccccc2)cc1F] | (2S,4R)-4-fluoro-N-[(S)-[3-fluoro- 4-(propan-2- yl)phenyl](phenyl)methyl]-1-{2-[4- (trifluoromethyl)-1H-1,2,3-triazol- 1-yl]acetyl} pyrrolidine-2- carboxamide |
| GZ-373 | 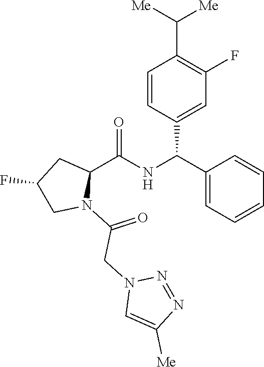 [SMILES: Cc1cn(CC(=O)N2C[C@H](F)C[C@H]2C(=O)N[C@@H](c2ccccc2)c2ccc(C(C)C)c(F)c2)nn1] | (2S,4R)-4-fluoro-N-[(S)-[3-fluoro- 4-(propan-2- yl)phenyl](phenyl)methyl]-1-[2-(4- methyl-1H-1,2,3-triazol-1- yl)acetyl]pyrrolidine-2- carboxamide |
| GZ-374 | 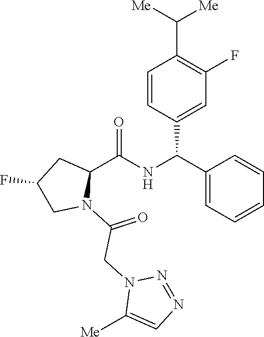 [SMILES: Cc1cnnn1CC(=O)N1C[C@H](F)C[C@H]1C(=O)N[C@@H](c1ccccc1)c1ccc(C(C)C)c(F)c1] | (2S,4R)-4-fluoro-N-[(S)-[3-fluoro- 4-(propan-2- yl)phenyl](phenyl)methyl]-1-[2-(5- methyl-1H-1,2,3-triazol-1- yl)acetyl]pyrrolidine-2- carboxamide |
| GZ-375 | 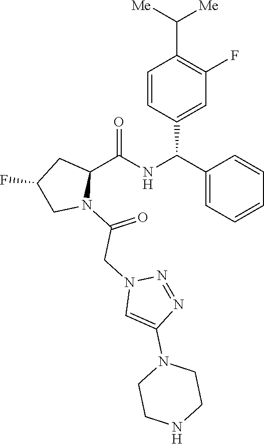 [SMILES: CC(C)c1ccc([C@@H](NC(=O)[C@@H]2C[C@@H](F)CN2C(=O)Cn2cc(N3CCNCC3)nn2)c2ccccc2)cc1F] | (2S,4R)-4-fluoro-N-[(S)-[3-fluoro- 4-(propan-2- yl)phenyl](phenyl)methyl]-1-{2-[4- (piperazin-1-yl)-1H-1,2,3-triazol-1- yl]acetyl} pyrrolidine-2- carboxamide |
| GZ-376 | 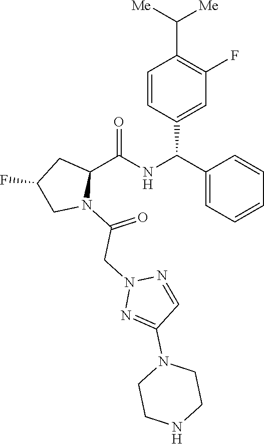 [SMILES: CC(C)c1ccc([C@@H](NC(=O)[C@@H]2C[C@@H](F)CN2C(=O)Cn2ncc(N3CCNCC3)n2)c2ccccc2)cc1F] | (2S,4R)-4-fluoro-N-[(S)-[3-fluoro- 4-(propan-2- yl)phenyl](phenyl)methyl]-1-{2-[4- (piperazin-1-yl)-2H-1,2,3-triazol-2- yl]acetyl } pyrrolidine-2- carboxamide |
| GZ-377 | 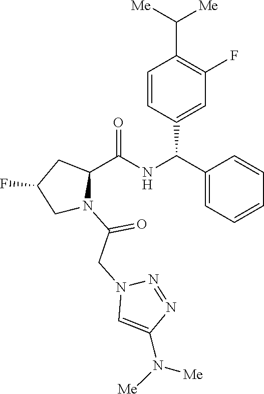 [SMILES: CC(C)c1ccc([C@@H](NC(=O)[C@@H]2C[C@@H](F)CN2C(=O)Cn2cc(N(C)C)nn2)c2ccccc2)cc1F] | (2S,4R)-1-{2-[4-(dimethylamino)- 1H-1,2,3-triazol-1-yl]acetyl}-4- fluoro-N-[(S)-[3-fluoro-4-(propan- 2- yl)phenyl](phenyl)methyl]pyrrolidine- 2-carboxamide |
| GZ-378 | 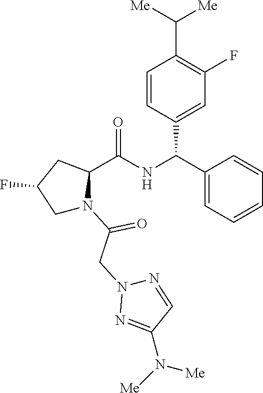 [SMILES: CC(C)c1ccc([C@@H](NC(=O)[C@@H]2C[C@@H](F)CN2C(=O)Cn2ncc(N(C)C)n2)c2ccccc2)cc1F] | (2S,4R)-1-{2-[4-(dimethylamino)- 2H-1,2,3-triazol-2-yl]acetyl}-4- fluoro-N-[(S)-[3-fluoro-4-(propan- 2- yl)phenyl](phenyl)methyl]pyrrolidine- 2-carboxamide |
| GZ-379 | 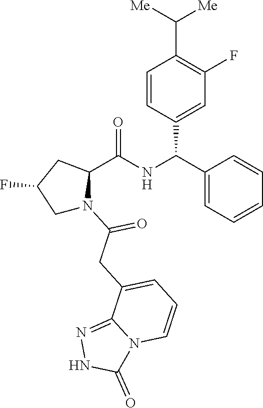 [SMILES: CC(C)c1ccc([C@@H](NC(=O)[C@@H]2C[C@@H](F)CN2C(=O)Cc2cccn3c(=O)[nH]nc23)c2ccccc2)cc1F] | (2S,4R)-4-fluoro-N-[(S)-[3-fluoro- 4-(propan-2- yl)phenyl](phenyl)methyl]-1-(2-{3- oxo-2H,3H-[1,2,4]triazolo[4,3- alpyridin-8-yl} acetyl)pyrrolidine-2- carboxamide |
| GZ-380 | 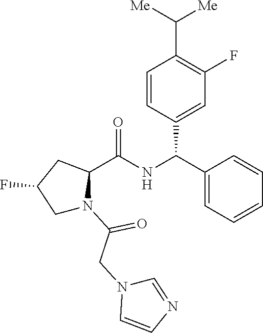 [SMILES: CC(C)c1ccc([C@@H](NC(=O)[C@@H]2C[C@@H](F)CN2C(=O)Cn2ccnc2)c2ccccc2)cc1F] | (2S,4R)-4-fluoro-N-[(S)-[3-fluoro- 4-(propan-2- yl)phenyl](phenyl)methyl]-1-[2- (1H-imidazol-1- yl)acetyl]pyrrolidine-2- carboxamide |
| GZ-381 | 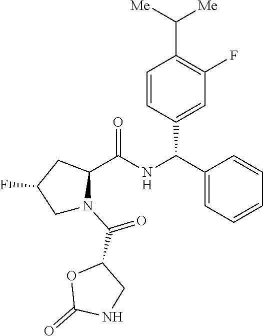 [SMILES: CC(C)c1ccc([C@@H](NC(=O)[C@@H]2C[C@@H](F)CN2C(=O)[C@@H]2CNC(=O)O2)c2ccccc2)cc1F] | (2S,4R)-4-fluoro-N-[(S)-[3-fluoro- 4-(propan-2- yl)phenyl](phenyl)methyl]-1-[(5S)- 2-oxo-1,3-oxazolidine-5- carbonyl]pyrrolidine-2- carboxamide |
| GZ-382 | 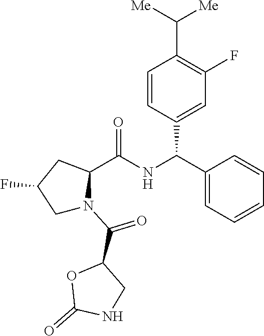 [SMILES: CC(C)c1ccc([C@@H](NC(=O)[C@@H]2C[C@@H](F)CN2C(=O)[C@H]2CNC(=O)O2)c2ccccc2)cc1F] | (2S,4R)-4-fluoro-N-[(S)-[3-fluoro- 4-(propan-2- yl)phenyl](phenyl)methyl]-1-[(5R)- 2-oxo-1,3-oxazolidine-5- carbonyl]pyrrolidine-2- carboxamide |
| GZ-383 | 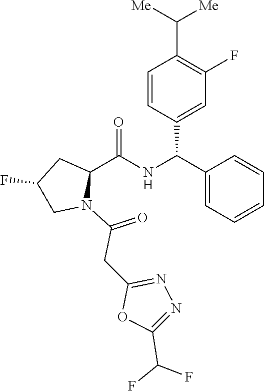 [SMILES: CC(C)c1ccc([C@@H](NC(=O)[C@@H]2C[C@@H](F)CN2C(=O)Cc2nnc(C(F)F)o2)c2ccccc2)cc1F] | (2S,4R)-1-{2-[5-(difluoromethyl)- 1,3,4-oxadiazol-2-yl]acetyl}-4- fluoro-N-[(S)-[3-fluoro-4-(propan- 2- yl)phenyl](phenyl)methyl]pyrrolidine- 2-carboxamide |
| GZ-384 | 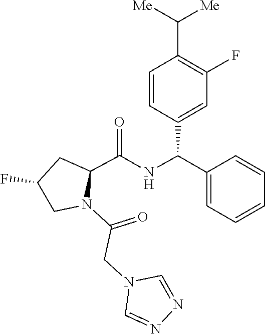 [SMILES: CC(C)c1ccc([C@@H](NC(=O)[C@@H]2C[C@@H](F)CN2C(=O)Cn2cnnc2)c2ccccc2)cc1F] | (2S,4R)-4-fluoro-N-[(S)-[3-fluoro- 4-(propan-2- yl)phenyl](phenyl)methyl]-1-[2- (4H-1,2,4-triazol-4- yl)acetyl]pyrrolidine-2- carboxamide |
| GZ-385 | 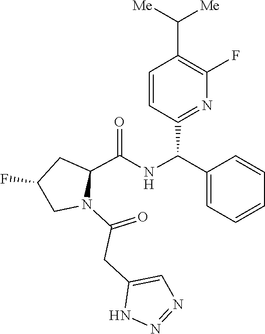 [SMILES: CC(C)c1ccc([C@@H](NC(=O)[C@@H]2C[C@@H](F)CN2C(=O)Cc2cnn[nH]2)c2ccccc2)nc1F] | (2S,4R)-1-(2-(1H-1,2,3-triazol-5- yl)acetyl)-4-fluoro-N-((S)-(6- fluoro-5-isopropylpyridin-2- yl)(phenyl)methyl)pyrrolidine-2- carboxamide |
| GZ-386 | 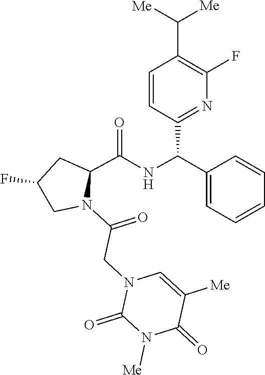 [SMILES: Cc1cn(CC(=O)N2C[C@H](F)C[C@H]2C(=O)N[C@@H](c2ccccc2)c2ccc(C(C)C)c(F)n2)c(=O)n(C)c1=O] | (2S,4R)-1-[2-(3,5-dimethyl-2,4- dioxo-1,2,3,4-tetrahydropyrimidin- 1-yl)acetyl]-4-fluoro-N-[(S)-[6- fluoro-5-(propan-2-yl)pyridin-2- yl](phenyl)methyl]pyrrolidine-2- carboxamide |
| GZ-387 | 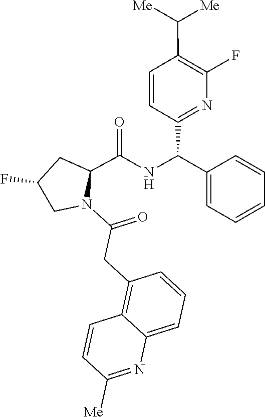 [SMILES: Cc1ccc2c(CC(=O)N3C[C@H](F)C[C@H]3C(=O)N[C@@H](c3ccccc3)c3ccc(C(C)C)c(F)n3)cccc2n1] | (2S,4R)-4-fluoro-N-[(S)-[6-fluoro- 5-(propan-2-yl)pyridin-2- yl](phenyl)methyl]-1-[2-(2- methylquinolin-5- yl)acetyl]pyrrolidine-2- carboxamide |
| GZ-388 | 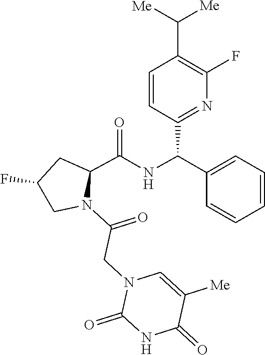 [SMILES: Cc1cn(CC(=O)N2C[C@H](F)C[C@H]2C(=O)N[C@@H](c2ccccc2)c2ccc(C(C)C)c(F)n2)c(=O)[nH]c1=O] | (2S,4R)-4-fluoro-N-[(S)-[6-fluoro- 5-(propan-2-yl)pyridin-2- yl](phenyl)methyl]-1-[2-(5-methyl- 2,4-dioxo-1,2,3,4- tetrahydropyrimidin-1- yl)acetyl]pyrrolidine-2- carboxamide |
| GZ-389 | 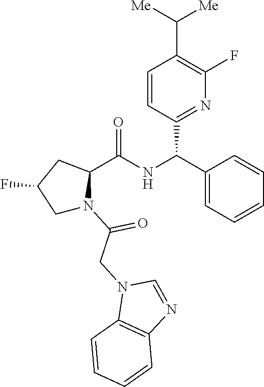 [SMILES: CC(C)c1ccc([C@@H](NC(=O)[C@@H]2C[C@@H](F)CN2C(=O)Cn2cnc3ccccc32)c2ccccc2)nc1F] | (2S,4R)-1-[2-(1H-1,3-benzodiazol- 1-yl)acetyl]-4-fluoro-N-[(S)-[6- fluoro-5-(propan-2-yl)pyridin-2- yl](phenyl)methyl]pyrrolidine-2- carboxamide |
| GZ-390 | 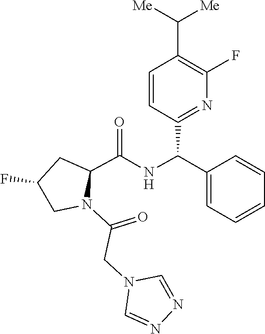 [SMILES: CC(C)c1ccc([C@@H](NC(=O)[C@@H]2C[C@@H](F)CN2C(=O)Cn2cnnc2)c2ccccc2)nc1F] | (2S,4R)-4-fluoro-N-[(S)-[6-fluoro- 5-(propan-2-yl)pyridin-2- yl](phenyl)methyl]-1-[2-(4H-1,2,4- triazol-4-yl)acetyl]pyrrolidine-2- carboxamide |
| GZ-391 | 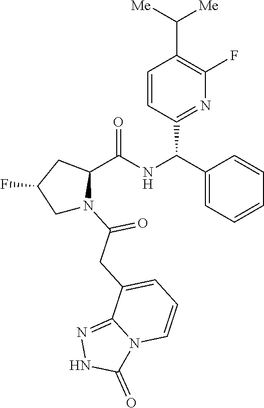 [SMILES: CC(C)c1ccc([C@@H](NC(=O)[C@@H]2C[C@@H](F)CN2C(=O)Cc2cccn3c(=O)[nH]nc23)c2ccccc2)nc1F] | (2S,4R)-4-fluoro-N-[(S)-[6-fluoro- 5-(propan-2-yl)pyridin-2- yl](phenyl)methyl]-1-(2-{3-oxo- 2H,3H-[1,2,4]triazolo[4,3- alpyridin-8-yl} acetyl)pyrrolidine-2- carboxamide |
| GZ-392 | 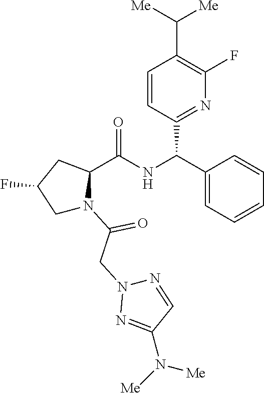 [SMILES: CC(C)c1ccc([C@@H](NC(=O)[C@@H]2C[C@@H](F)CN2C(=O)Cn2ncc(N(C)C)n2)c2ccccc2)nc1F] | (2S,4R)-1-{2-[4-(dimethylamino)- 2H-1,2,3-triazol-2-yl]acetyl}-4- fluoro-N-[(S)-[6-fluoro-5-(propan- 2-yl)pyridin-2- yl](phenyl)methyl]pyrrolidine-2- carboxamide |
| GZ-393 | 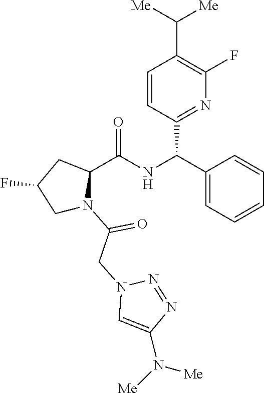 [SMILES: CC(C)c1ccc([C@@H](NC(=O)[C@@H]2C[C@@H](F)CN2C(=O)Cn2cc(N(C)C)nn2)c2ccccc2)nc1F] | (2S,4R)-1-{2-[4-(dimethylamino)- 1H-1,2,3-triazol-1-yl]acetyl}-4- fluoro-N-[(S)-[6-fluoro-5-(propan- 2-yl)pyridin-2- yl](phenyl)methyl]pyrrolidine-2- carboxamide |
| GZ-394 | 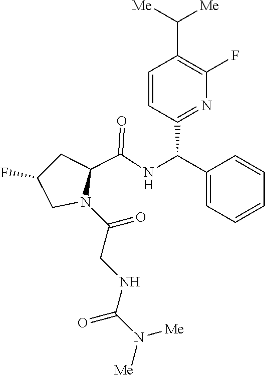 [SMILES: CC(C)c1ccc([C@@H](NC(=O)[C@@H]2C[C@@H](F)CN2C(=O)CNC(=O)N(C)C)c2ccccc2)nc1F] | (2S,4R)-1-{2- [(dimethylcarbamoyl)amino]acetyl}- 4-fluoro-N-[(S)-[6-fluoro-5- (propan-2-yl)pyridin-2- yl](phenyl)methyl]pyrrolidine-2- carboxamide |
| GZ-395 | 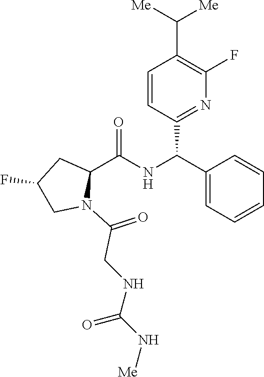 [SMILES: CNC(=O)NCC(=O)N1C[C@H](F)C[C@H]1C(=O)N[C@@H](c1ccccc1)c1ccc(C(C)C)c(F)n1] | (2S,4R)-4-fluoro-N-[(S)-[6-fluoro- 5-(propan-2-yl)pyridin-2- yl](phenyl)methyl]-1-{2- [(methylcarbamoyl)amino]acetyl} pyrrolidine-2-carboxamide |
| GZ-396 | 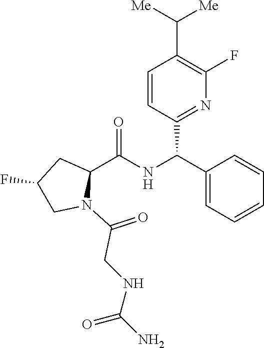 [SMILES: CC(C)c1ccc([C@@H](NC(=O)[C@@H]2C[C@@H](F)CN2C(=O)CNC(N)=O)c2ccccc2)nc1F] | (2S,4R)-1-[2- (carbamoylamino)acetyl]-4-fluoro- N-[(S)-[6-fluoro-5-(propan-2- yl)pyridin-2- yl](phenyl)methyl]pyrrolidine-2- carboxamide |
| GZ-397 | 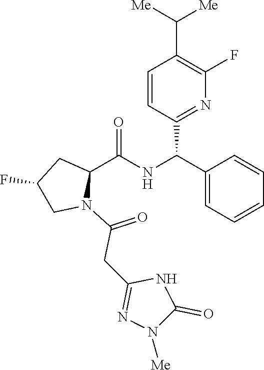 [SMILES: CC(C)c1ccc([C@@H](NC(=O)[C@@H]2C[C@@H](F)CN2C(=O)Cc2nn(C)c(=O)[nH]2)c2ccccc2)nc1F] | (2S,4R)-4-fluoro-N-[(S)-[6-fluoro- 5-(propan-2-yl)pyridin-2- yl](phenyl)methyl]-1-[2-(1-methyl- 5-oxo-4,5-dihydro-1H-1,2,4-triazol- 3-yl)acetyl]pyrrolidine-2- carboxamide |
| GZ-398 | 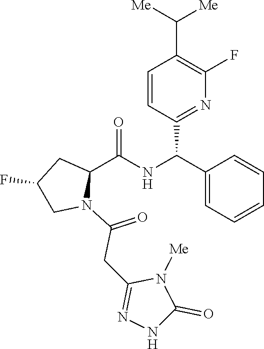 [SMILES: CC(C)c1ccc([C@@H](NC(=O)[C@@H]2C[C@@H](F)CN2C(=O)Cc2n[nH]c(=O)n2C)c2ccccc2)nc1F] | (2S,4R)-4-fluoro-N-[(S)-[6-fluoro- 5-(propan-2-yl)pyridin-2- yl](phenyl)methyl]-1-[2-(4-methyl- 5-oxo-4,5-dihydro-1H-1,2,4-triazol- 3-yl)acetyl]pyrrolidine-2- carboxamide |
| GZ-399 | 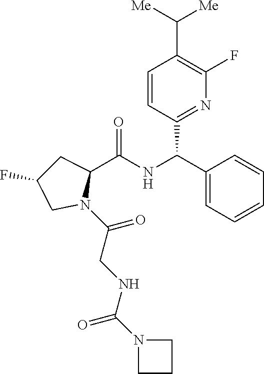 [SMILES: CC(C)c1ccc([C@@H](NC(=O)[C@@H]2C[C@@H](F)CN2C(=O)CNC(=O)N2CCC2)c2ccccc2)nc1F] | (2S,4R)-1-{2-[(azetidine-1- carbonyl)amino]acetyl}-4-fluoro- N-[(S)-[6-fluoro-5-(propan-2- yl)pyridin-2- yl](phenyl)methyl]pyrrolidine-2- carboxamide |
| GZ-400 | 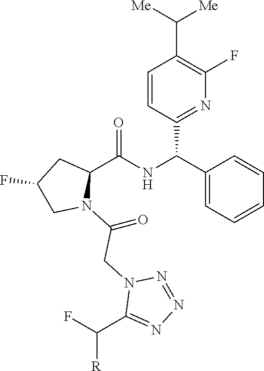 | (2S,4R)-1-{2-[5-(difluoromethyl)- 1H-1,2,3,4-tetrazol-1-yl]acetyl}-4- fluoro-N-[(S)-[6-fluoro-5-(propan- 2-yl)pyridin-2- yl](phenyl)methyl]pyrrolidine-2- carboxamide |
| GZ-401 | 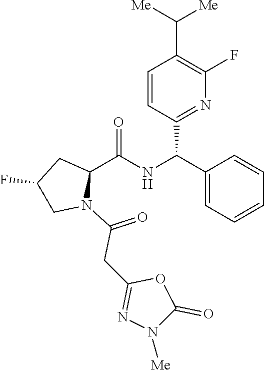 [SMILES: CC(C)c1ccc([C@@H](NC(=O)[C@@H]2C[C@@H](F)CN2C(=O)Cc2nn(C)c(=O)o2)c2ccccc2)nc1F] | (2S,4R)-4-fluoro-N-[(S)-[6-fluoro- 5-(propan-2-yl)pyridin-2- yl](phenyl)methyl]-1-[2-(4-methyl- 5-oxo-4,5-dihydro-1,3,4-oxadiazol- 2-yl)acetyl]pyrrolidine-2- carboxamide |
| GZ-402 | 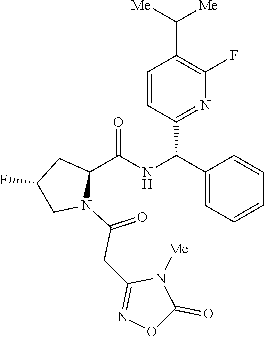 [SMILES: CC(C)c1ccc([C@@H](NC(=O)[C@@H]2C[C@@H](F)CN2C(=O)Cc2noc(=O)n2C)c2ccccc2)nc1F] | (2S,4R)-4-fluoro-N-[(S)-[6-fluoro- 5-(propan-2-yl)pyridin-2- yl](phenyl)methyl]-1-[2-(4-methyl- 5-oxo-4,5-dihydro-1,2,4-oxadiazol- 3-yl)acetyl]pyrrolidine-2- carboxamide |
| GZ-403 | 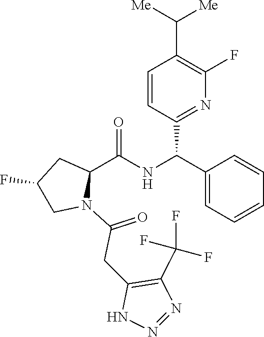 [SMILES: CC(C)c1ccc([C@@H](NC(=O)[C@@H]2C[C@@H](F)CN2C(=O)Cc2[nH]nnc2C(F)(F)F)c2ccccc2)nc1F] | (2S,4R)-4-fluoro-N-[(S)-[6-fluoro- 5-(propan-2-yl)pyridin-2- yl](phenyl)methyl]-1-{2-[4- (trifluoromethyl)-1H-1,2,3-triazol- 5-yl]acetyl } pyrrolidine-2- carboxamide |
| GZ-404 | 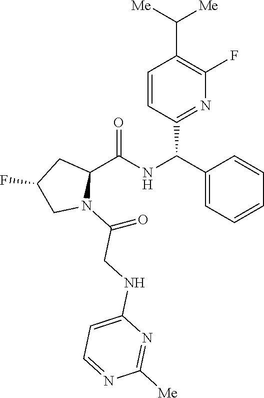 [SMILES: Cc1nccc(NCC(=O)N2C[C@H](F)C[C@H]2C(=O)N[C@@H](c2ccccc2)c2ccc(C(C)C)c(F)n2)n1] | (2S,4R)-4-fluoro-N-[(S)-[6-fluoro- 5-(propan-2-yl)pyridin-2- yl](phenyl)methyl]-1-{2-[(2- methylpyrimidin-4- yl)amino]acetyl} pyrrolidine-2- carboxamide |
| GZ-405 | 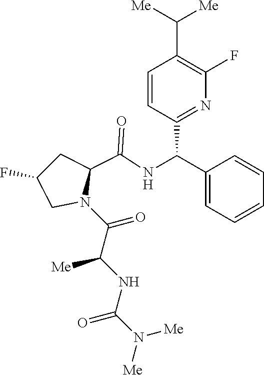 [SMILES: CC(C)c1ccc([C@@H](NC(=O)[C@@H]2C[C@@H](F)CN2C(=O)[C@H](C)NC(=O)N(C)C)c2ccccc2)nc1F] | (2S,4R)-1-[(2S)-2- [(dimethylcarbamoyl)amino] propanoyl]-4-fluoro-N-[(S)-[6-fluoro-5- (propan-2-yl)pyridin-2- yl](phenyl)methyl]pyrrolidine-2- carboxamide |
| GZ-406 | 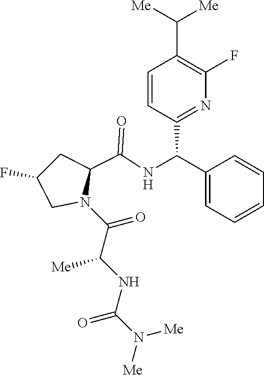 [SMILES: CC(C)c1ccc([C@@H](NC(=O)[C@@H]2C[C@@H](F)CN2C(=O)[C@@H](C)NC(=O)N(C)C)c2ccccc2)nc1F] | (2S,4R)-1-[(2R)-2- [(dimethylcarbamoyl)amino] propanoyl]-4-fluoro-N-[(S)-[6-fluoro-5- (propan-2-yl)pyridin-2- yl](phenyl)methyl]pyrrolidine-2- carboxamide |
| GZ-407 | 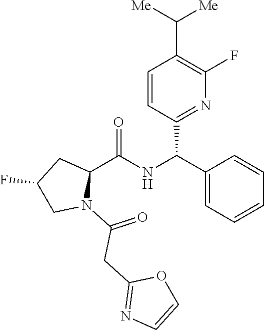 [SMILES: CC(C)c1ccc([C@@H](NC(=O)[C@@H]2C[C@@H](F)CN2C(=O)Cc2ncco2)c2ccccc2)nc1F] | (2S,4R)-4-fluoro-N-[(S)-[6-fluoro- 5-(propan-2-yl)pyridin-2- yl](phenyl)methyl]-1-[2-(1,3- oxazol-2-yl)acetyl]pyrrolidine-2- carboxamide |
| GZ-408 | 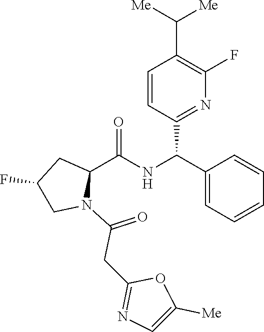 [SMILES: Cc1cnc(CC(=O)N2C[C@H](F)C[C@H]2C(=O)N[C@@H](c2ccccc2)c2ccc(C(C)C)c(F)n2)o1] | (2S,4R)-4-fluoro-N-[(S)-[6-fluoro- 5-(propan-2-yl)pyridin-2- yl](phenyl)methyl]-1-[2-(5-methyl- 1,3-oxazol-2-yl)acetyl]pyrrolidine- 2-carboxamide |
| GZ-409 | 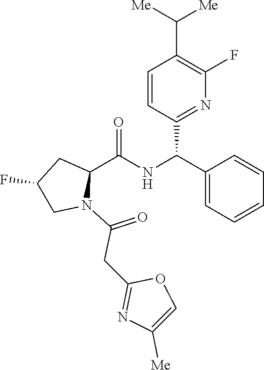 [SMILES: Cc1coc(CC(=O)N2C[C@H](F)C[C@H]2C(=O)N[C@@H](c2ccccc2)c2ccc(C(C)C)c(F)n2)n1] | (2S,4R)-4-fluoro-N-[(S)-[6-fluoro- 5-(propan-2-yl)pyridin-2- yl](phenyl)methyl]-1-[2-(4-methyl- 1,3-oxazol-2-yl)acetyl]pyrrolidine- 2-carboxamide |
| GZ-410 | 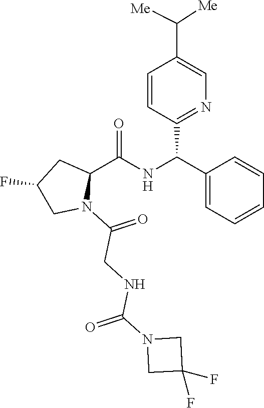 [SMILES: CC(C)c1ccc([C@@H](NC(=O)[C@@H]2C[C@@H](F)CN2C(=O)CNC(=O)N2CC(F)(F)C2)c2ccccc2)nc1] | (2S,4R)-1-{2-[(3,3- difluoroazetidine-1- carbonyl)amino]acetyl}-4-fluoro- N-[(S)-[6-fluoro-5-(propan-2- yl)pyridin-2- yl](phenyl)methyl]pyrrolidine-2- carboxamide |
| GZ-411 | 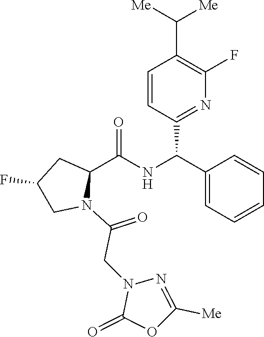 [SMILES: Cc1nn(CC(=O)N2C[C@H](F)C[C@H]2C(=O)N[C@@H](c2ccccc2)c2ccc(C(C)C)c(F)n2)c(=O)o1] | (2S,4R)-4-fluoro-N-[(S)-[6-fluoro- 5-(propan-2-yl)pyridin-2- yl](phenyl)methyl]-1-[2-(5-methyl- 2-oxo-2,3-dihydro-1,3,4-oxadiazol- 3-yl)acetyl]pyrrolidine-2- carboxamide |
| GZ-412 | 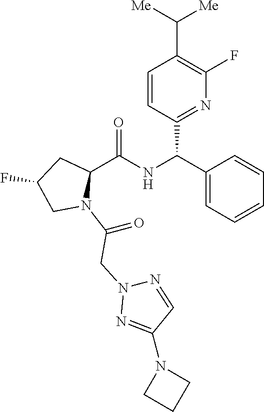 [SMILES: CC(C)c1ccc([C@@H](NC(=O)[C@@H]2C[C@@H](F)CN2C(=O)Cn2ncc(N3CCC3)n2)c2ccccc2)nc1F] | (2S,4R)-1-{2-[4-(azetidin-1-yl)-2H- 1,2,3-triazol-2-yl]acetyl}-4-fluoro- N-[(S)-[6-fluoro-5-(propan-2- yl)pyridin-2- yl](phenyl)methyl]pyrrolidine-2- carboxamide |
| GZ-413 | 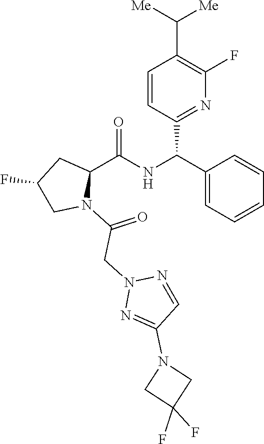 [SMILES: CC(C)c1ccc([C@@H](NC(=O)[C@@H]2C[C@@H](F)CN2C(=O)Cn2ncc(N3CC(F)(F)C3)n2)c2ccccc2)nc1F] | (2S,4R)-1-{2-[4-(3,3- difluoroazetidin-1-yl)-2H-1,2,3- triazol-2-yl]acetyl}-4-fluoro-N- [(S)-[6-fluoro-5-(propan-2- yl)pyridin-2- yl](phenyl)methyl]pyrrolidine-2- carboxamide |
| GZ-414 | 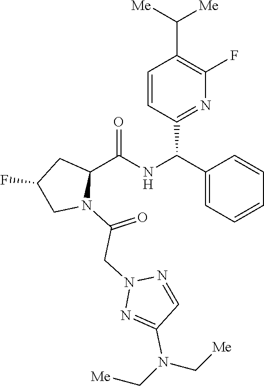 [SMILES: CCN(CC)c1cnn(CC(=O)N2C[C@H](F)C[C@H]2C(=O)N[C@@H](c2ccccc2)c2ccc(C(C)C)c(F)n2)n1] | (2S,4R)-1-{2-[4-(diethylamino)- 2H-1,2,3-triazol-2-yl]acetyl}-4- fluoro-N-[(S)-[6-fluoro-5-(propan- 2-yl)pyridin-2- yl](phenyl)methyl]pyrrolidine-2- carboxamide |
| GZ-415 | 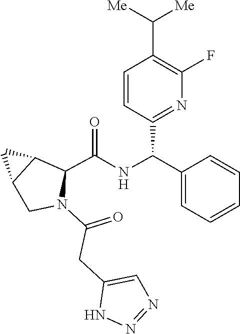 [SMILES: CC(C)c1ccc([C@@H](NC(=O)[C@@H]2[C@H]3C[C@H]3CN2C(=O)Cc2cnn[nH]2)c2ccccc2)nc1F] | (1S,2S,5R)-N-[(S)-[6-fluoro-5- (propan-2-yl)pyridin-2- yl](phenyl)methyl]-3-[2-(1H-1,2,3- triazol-5-yl)acetyl]-3- azabicyclo[3.1.0]hexane-2- carboxamide |
| GZ-416 | 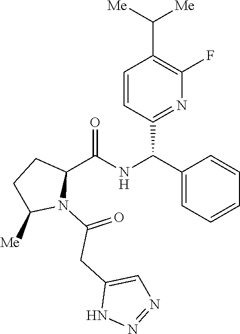 [SMILES: CC(C)c1ccc([C@@H](NC(=O)[C@@H]2CC[C@H](C)N2C(=O)Cc2cnn[nH]2)c2ccccc2)nc1F] | (2S,5S)-N-[(S)-[6-fluoro-5- (propan-2-yl)pyridin-2- yl](phenyl)methyl]-5-methyl-1-[2- (1H-1,2,3-triazol-5- yl)acetyl]pyrrolidine-2- carboxamide |
| GZ-417 | 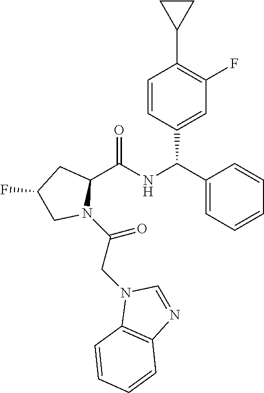 [SMILES: O=C(N[C@@H](c1ccccc1)c1ccc(C2CC2)c(F)c1)[C@@H]1C[C@@H](F)CN1C(=O)Cn1cnc2ccccc21] | (2S,4R)-1-(2-(1H- benzo[d]imidazol-1-yl)acetyl)-N- ((S)-(4-cyclopropyl-3- fluorophenyl)(phenyl)methyl)-4- fluoropyrrolidine-2-carboxamide |
| GZ-418 | 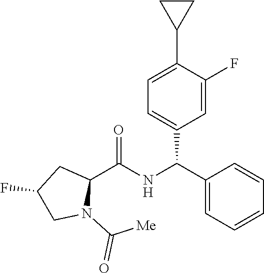 [SMILES: CC(=O)N1C[C@H](F)C[C@H]1C(=O)N[C@@H](c1ccccc1)c1ccc(C2CC2)c(F)c1] | (2S,4R)-1-acetyl-N-[(S)-(4- cyclopropyl-3- fluorophenyl)(phenyl)methyl]-4- fluoropyrrolidine-2-carboxamide |
| GZ-419 | 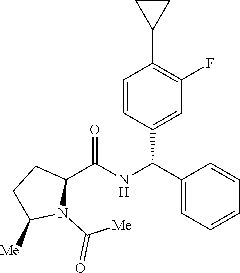 [SMILES: CC(=O)N1[C@@H](C)CC[C@H]1C(=O)N[C@@H](c1ccccc1)c1ccc(C2CC2)c(F)c1] | (2S,5S)-1-acetyl-N-[(S)-(4- cyclopropyl-3- fluorophenyl)(phenyl)methyl]-5- methylpyrrolidine-2-carboxamide |
| GZ-420 | 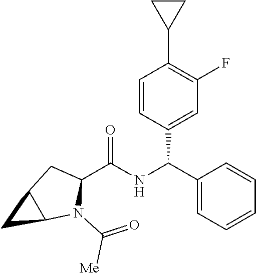 [SMILES: CC(=O)N1[C@H](C(=O)N[C@@H](c2ccccc2)c2ccc(C3CC3)c(F)c2)C[C@@H]2C[C@@H]21] | (1S,3S,5S)-2-acetyl-N-[(S)-(4- cyclopropyl-3- fluorophenyl)(phenyl)methyl]-2- azabicyclo[3.1.0]hexane-3- carboxamide |
| GZ-421 | 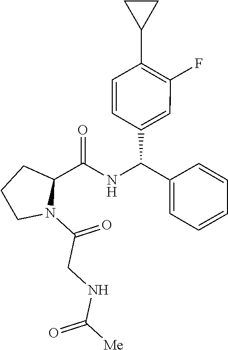 [SMILES: CC(=O)NCC(=O)N1CCC[C@H]1C(=O)N[C@@H](c1ccccc1)c1ccc(C2CC2)c(F)c1] | (2S)-N-[(S)-(4-cyclopropyl-3- fluorophenyl)(phenyl)methyl]-1-(2- acetamidoacetyl)pyrrolidine-2- carboxamide |
| GZ-422 | 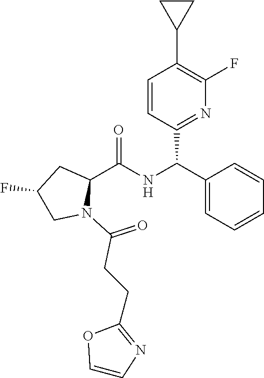 [SMILES: O=C(N[C@@H](c1ccccc1)c1ccc(C2CC2)c(F)n1)[C@@H]1C[C@@H](F)CN1C(=O)CCc1ncco1] | (2S,4R)-N-[(S)-(4-cyclopropyl-3- fluorophenyl)(phenyl)methyl]-4- fluoro-1-[3-(1,3-oxazol-2- yl)propanoyl]pyrrolidine-2- carboxamide |
| GZ-423 | 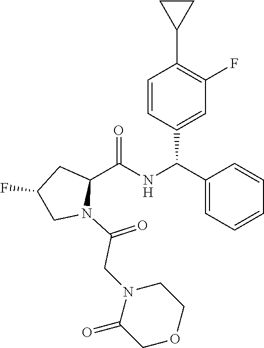 [SMILES: O=C(N[C@@H](c1ccccc1)c1ccc(C2CC2)c(F)c1)[C@@H]1C[C@@H](F)CN1C(=O)CN1CCOCC1=O] | (2S,4R)-N-[(S)-(4-cyclopropyl-3- fluorophenyl)(phenyl)methyl]-4- fluoro-1-[2-(3-oxomorpholin-4- yl)acetyl]pyrrolidine-2- carboxamide |
| GZ-424 | 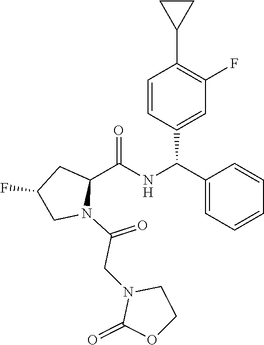 [SMILES: O=C(N[C@@H](c1ccccc1)c1ccc(C2CC2)c(F)c1)[C@@H]1C[C@@H](F)CN1C(=O)CN1CCOC1=O] | (2S,4R)-N-[(S)-(4-cyclopropyl-3- fluorophenyl)(phenyl)methyl]-4- fluoro-1-[2-(2-oxo-1,3-oxazolidin- 3-yl)acetyl]pyrrolidine-2- carboxamide |
| GZ-425 | 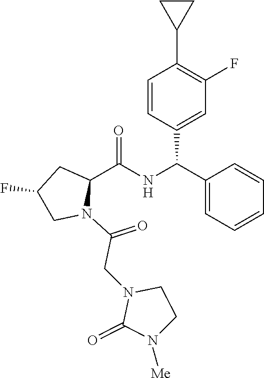 [SMILES: CN1CCN(CC(=O)N2C[C@H](F)C[C@H]2C(=O)N[C@@H](c2ccccc2)c2ccc(C3CC3)c(F)c2)C1=O] | (2S,4R)-N-[(S)-(4-cyclopropyl-3- fluorophenyl)(phenyl)methyl]-4- fluoro-1-[2-(3-methyl-2- oxoimidazolidin-1- yl)acetyl]pyrrolidine-2- carboxamide |
| GZ-426 | 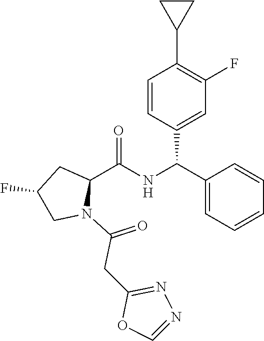 [SMILES: O=C(N[C@@H](c1ccccc1)c1ccc(C2CC2)c(F)c1)[C@@H]1C[C@@H](F)CN1C(=O)Cc1nnco1] | (2S,4R)-N-[(S)-(4-cyclopropyl-3- fluorophenyl)(phenyl)methyl]-4- fluoro-1-[2-(1,3,4-oxadiazol-2- yl)acetyl]pyrrolidine-2- carboxamide |
| GZ-427 | 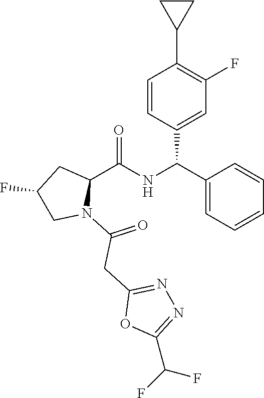 [SMILES: O=C(N[C@@H](c1ccccc1)c1ccc(C2CC2)c(F)c1)[C@@H]1C[C@@H](F)CN1C(=O)Cc1nnc(C(F)F)o1] | (2S,4R)-N-[(S)-(4-cyclopropyl-3- fluorophenyl)(phenyl)methyl]-1- {2-[5-(difluoromethyl)-1,3,4- oxadiazol-2-yl]acetyl}-4- fluoropyrrolidine-2-carboxamide |
| GZ-428 | 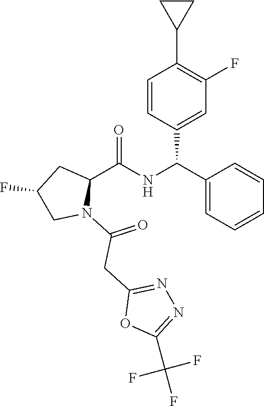 [SMILES: O=C(N[C@@H](c1ccccc1)c1ccc(C2CC2)c(F)c1)[C@@H]1C[C@@H](F)CN1C(=O)Cc1nnc(C(F)(F)F)o1] | (2S,4R)-N-[(S)-(4-cyclopropyl-3- fluorophenyl)(phenyl)methyl]-4- fluoro-1-{2-[5-(trifluoromethyl)- 1,3,4-oxadiazol-2- yl]acetyl} pyrrolidine-2- carboxamide |
| GZ-429 | 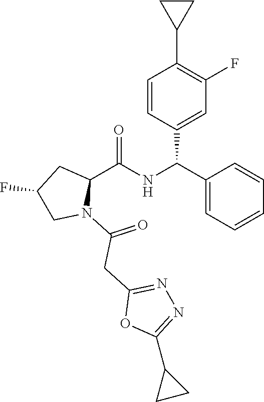 [SMILES: O=C(N[C@@H](c1ccccc1)c1ccc(C2CC2)c(F)c1)[C@@H]1C[C@@H](F)CN1C(=O)Cc1nnc(C2CC2)o1] | (2S,4R)-1-[2-(5-cyclopropyl-1,3,4- oxadiazol-2-yl)acetyl]-N-[(S)-(4- cyclopropyl-3- fluorophenyl)(phenyl)methyl]-4- fluoropyrrolidine-2-carboxamide |
| GZ-430 | 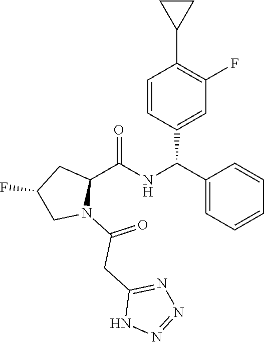 [SMILES: O=C(N[C@@H](c1ccccc1)c1ccc(C2CC2)c(F)c1)[C@@H]1C[C@@H](F)CN1C(=O)Cc1nnn[nH]1] | (2S,4R)-N-[(S)-(4-cyclopropyl-3- fluorophenyl)(phenyl)methyl]-4- fluoro-1-[2-(1H-1,2,3,4-tetrazol-5- yl)acetyl]pyrrolidine-2- carboxamide |
| GZ-431 | 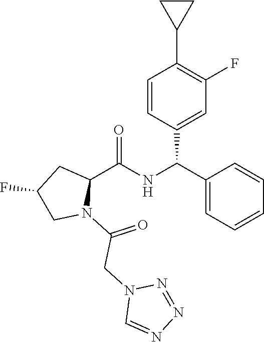 [SMILES: O=C(N[C@@H](c1ccccc1)c1ccc(C2CC2)c(F)c1)[C@@H]1C[C@@H](F)CN1C(=O)Cn1cnnn1] | (2S,4R)-N-[(S)-(4-cyclopropyl-3- fluorophenyl)(phenyl)methyl]-4- fluoro-1-[2-(1H-1,2,3,4-tetrazol-1- yl)acetyl]pyrrolidine-2- carboxamide |
| GZ-432 | 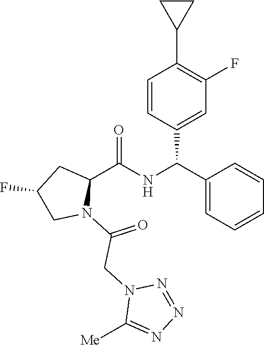 [SMILES: Cc1nnnn1CC(=O)N1C[C@H](F)C[C@H]1C(=O)N[C@@H](c1ccccc1)c1ccc(C2CC2)c(F)c1] | (2S,4R)-N-[(S)-(4-cyclopropyl-3- fluorophenyl)(phenyl)methyl]-4- fluoro-1-[2-(5-methyl-1H-1,2,3,4- tetrazol-1-yl)acetyl]pyrrolidine-2- carboxamide |
| GZ-433 | 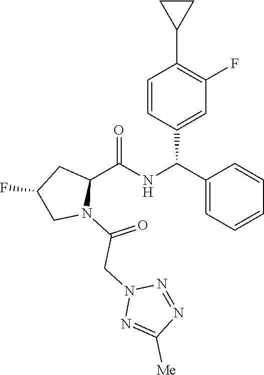 [SMILES: Cc1nnn(CC(=O)N2C[C@H](F)C[C@H]2C(=O)N[C@@H](c2ccccc2)c2ccc(C3CC3)c(F)c2)n1] | (2S,4R)-N-[(S)-(4-cyclopropyl-3- fluorophenyl)(phenyl)methyl]-4- fluoro-1-[2-(5-methyl-2H-1,2,3,4- tetrazol-2-yl)acetyl]pyrrolidine-2- carboxamide |
| GZ-434 | 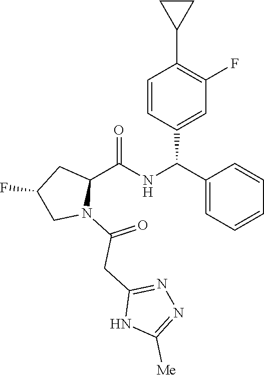 [SMILES: Cc1nnc(CC(=O)N2C[C@H](F)C[C@H]2C(=O)N[C@@H](c2ccccc2)c2ccc(C3CC3)c(F)c2)[nH]1] | (2S,4R)-N-[(S)-(4-cyclopropyl-3- fluorophenyl)(phenyl)methyl]-4- fluoro-1-[2-(5-methyl-4H-1,2,4- triazol-3-yl)acetyl]pyrrolidine-2- carboxamide |
| GZ-435 | 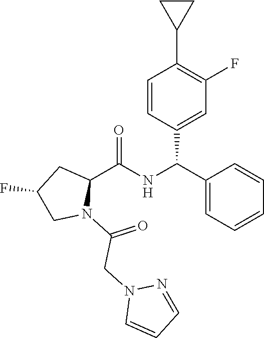 [SMILES: O=C(N[C@@H](c1ccccc1)c1ccc(C2CC2)c(F)c1)[C@@H]1C[C@@H](F)CN1C(=O)Cn1cccn1] | (2S,4R)-N-[(S)-(4-cyclopropyl-3- fluorophenyl)(phenyl)methyl]-4- fluoro-1-[2-(1H-pyrazol-1- yl)acetyl]pyrrolidine-2- carboxamide |
| GZ-436 | 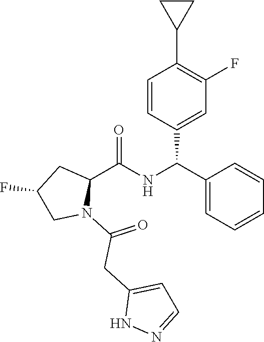 [SMILES: O=C(N[C@@H](c1ccccc1)c1ccc(C2CC2)c(F)c1)[C@@H]1C[C@@H](F)CN1C(=O)Cc1ccn[nH]1] | (2S,4R)-N-[(S)-(4-cyclopropyl-3- fluorophenyl)(phenyl)methyl]-4- fluoro-1-[2-(1H-pyrazol-5- yl)acetyl]pyrrolidine-2- carboxamide |
| GZ-437 | 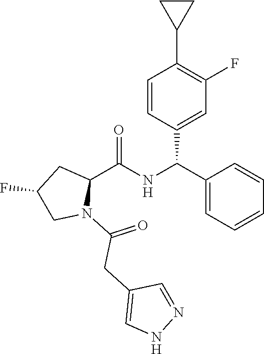 [SMILES: O=C(N[C@@H](c1ccccc1)c1ccc(C2CC2)c(F)c1)[C@@H]1C[C@@H](F)CN1C(=O)Cc1cn[nH]c1] | (2S,4R)-N-[(S)-(4-cyclopropyl-3- fluorophenyl)(phenyl)methyl]-4- fluoro-1-[2-(1H-pyrazol-4- yl)acetyl]pyrrolidine-2- carboxamide |
| GZ-438 | 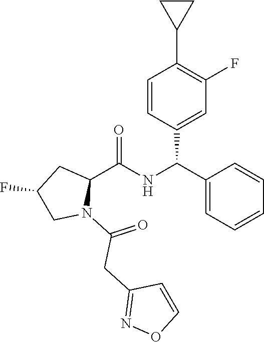 [SMILES: O=C(N[C@@H](c1ccccc1)c1ccc(C2CC2)c(F)c1)[C@@H]1C[C@@H](F)CN1C(=O)Cc1ccon1] | (2S,4R)-N-[(S)-(4-cyclopropyl-3- fluorophenyl)(phenyl)methyl]-4- fluoro-1-[2-(1,2-oxazol-3- yl)acetyl]pyrrolidine-2- carboxamide |
| GZ-439 | 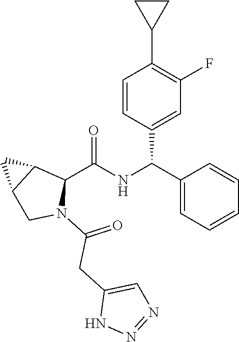 [SMILES: O=C(N[C@@H](c1ccccc1)c1ccc(C2CC2)c(F)c1)[C@@H]1[C@H]2C[C@H]2CN1C(=O)Cc1cnn[nH]1] | (2S,4R)-N-[(S)-(4-cyclopropyl-3- fluorophenyl)(phenyl)methyl]-4- fluoro-1-[2-(1H-1,2,3-triazol-5- yl)acetyl]pyrrolidine-2- carboxamide |
| GZ-440 | 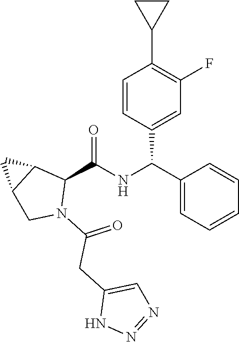 [SMILES: O=C(N[C@@H](c1ccccc1)c1ccc(C2CC2)c(F)c1)[C@@H]1[C@H]2C[C@H]2CN1C(=O)Cc1cnn[nH]1] | (1S,2S,5R)-N-[(S)-(4-cyclopropyl- 3-fluorophenyl)(phenyl)methyl]-3- [2-(1H-1,2,3-triazol-5-yl)acetyl]-3- azabicyclo[3.1.0]hexane-2- carboxamide |
| GZ-441 | 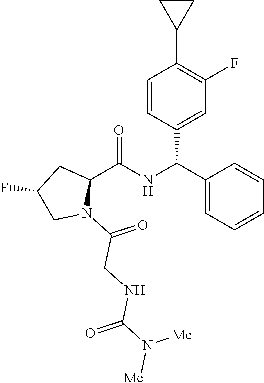 [SMILES: CN(C)C(=O)NCC(=O)N1C[C@H](F)C[C@H]1C(=O)N[C@@H](c1ccccc1)c1ccc(C2CC2)c(F)c1] | (2S,4R)-N-[(S)-(4-cyclopropyl-3- fluorophenyl)(phenyl)methyl]-1- {2- [(dimethylcarbamoyl)amino]acetyl}- 4-fluoropyrrolidine-2-carboxamide |
| GZ-442 | 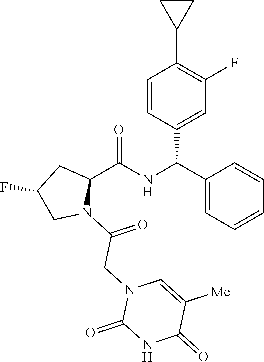 [SMILES: Cc1cn(CC(=O)N2C[C@H](F)C[C@H]2C(=O)N[C@@H](c2ccccc2)c2ccc(C3CC3)c(F)c2)c(=O)[nH]c1=O] | (2S,4R)-N-[(S)-(4-cyclopropyl-3- fluorophenyl)(phenyl)methyl]-4- fluoro-1-[2-(5-methyl-2,4-dioxo- 1,2,3,4-tetrahydropyrimidin-1- yl)acetyl]pyrrolidine-2- carboxamide |
| GZ-443 | 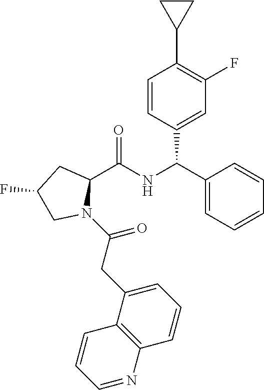 [SMILES: O=C(N[C@@H](c1ccccc1)c1ccc(C2CC2)c(F)c1)[C@@H]1C[C@@H](F)CN1C(=O)Cc1cccc2ncccc12] | (2S,4R)-N-[(S)-(4-cyclopropyl-3- fluorophenyl)(phenyl)methyl]-4- fluoro-1-[2-(quinolin-5- yl)acetyl]pyrrolidine-2- carboxamide |
| GZ-444 | 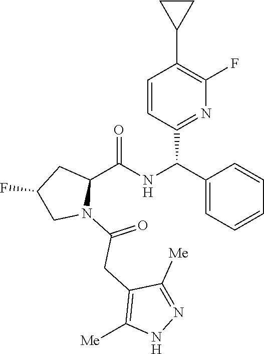 [SMILES: Cc1n[nH]c(C)c1CC(=O)N1C[C@H](F)C[C@H]1C(=O)N[C@@H](c1ccccc1)c1ccc(C2CC2)c(F)n1] | (2S,4R)-N-[(S)-(4-cyclopropyl-3- fluorophenyl)(phenyl)methyl]-1-[2- (3,5-dimethyl-1H-pyrazol-4- yl)acetyl]-4-fluoropyrrolidine-2- carboxamide |
| GZ-445 | 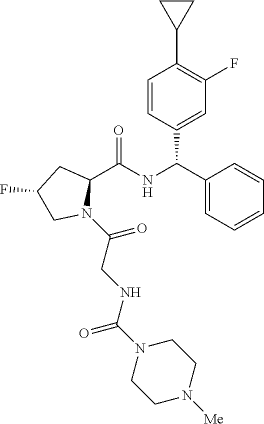 [SMILES: CN1CCN(C(=O)NCC(=O)N2C[C@H](F)C[C@H]2C(=O)N[C@@H](c2ccccc2)c2ccc(C3CC3)c(F)c2)CC1] | 2-[(2S,4R)-2-{[(S)-(4-cyclopropyl- 3- fluorophenyl)(phenyl)methyl] carbamoyl}-4-fluoropyrrolidin-1-yl]-2- oxoethyl 4-methylpiperazine-1- carboxylate |
| GZ-446 | 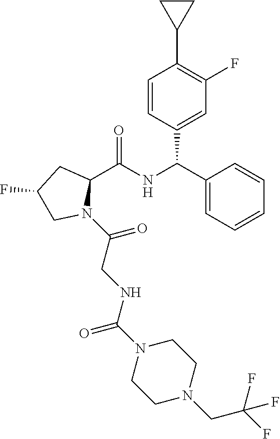 [SMILES: O=C(N[C@@H](c1ccccc1)c1ccc(C2CC2)c(F)c1)[C@@H]1C[C@@H](F)CN1C(=O)CNC(=O)N1CCN(CC(F)(F)F)CC1] | 2-[(2S,4R)-2-{[(S)-(4-cyclopropyl- 3- fluorophenyl)(phenyl)methyl] carbamoyl}-4-fluoropyrrolidin-1-yl]-2- oxoethyl 4-(2,2,2- trifluoroethyl)piperazine-1- carboxylate |
| GZ-447 | 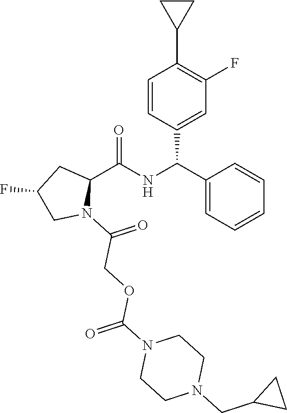 [SMILES: O=C(N[C@@H](c1ccccc1)c1ccc(C2CC2)c(F)c1)[C@@H]1C[C@@H](F)CN1C(=O)COC(=O)N1CCN(CC2CC2)CC1] | 2-[(2S,4R)-2-{[(S)-(4-cyclopropyl- 3- fluorophenyl)(phenyl)methyl] carbamoyl}-4-fluoropyrrolidin-1-yl]-2- oxoethyl 4- (cyclopropylmethyl)piperazine-1- carboxylate |
| GZ-448 | 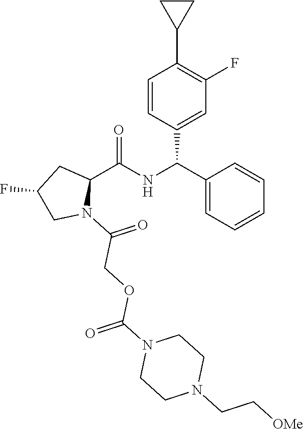 [SMILES: COCCN1CCN(C(=O)OCC(=O)N2C[C@H](F)C[C@H]2C(=O)N[C@@H](c2ccccc2)c2ccc(C3CC3)c(F)c2)CC1] | 2-[(2S,4R)-2-{[(S)-(4-cyclopropyl- 3- fluorophenyl)(phenyl)methyl] carbamoyl}-4-fluoropyrrolidin-1-yl]-2- oxoethyl 4-(2- methoxyethyl)piperazine-1- carboxylate |
| GZ-449 | 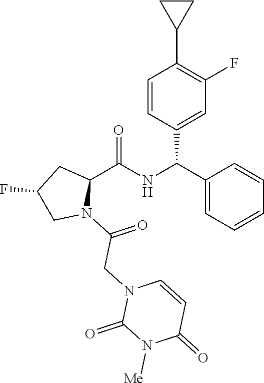 [SMILES: Cn1c(=O)ccn(CC(=O)N2C[C@H](F)C[C@H]2C(=O)N[C@@H](c2ccccc2)c2ccc(C3CC3)c(F)c2)c1=O] | (2S,4R)-N-[(S)-(4-cyclopropyl-3- fluorophenyl)(phenyl)methyl]-4- fluoro-1-[2-(3-methyl-2,4-dioxo- 1,2,3,4-tetrahydropyrimidin-1- yl)acetyl]pyrrolidine-2- carboxamide |
| GZ-450 | 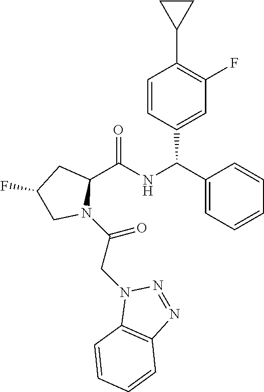 [SMILES: O=C(N[C@@H](c1ccccc1)c1ccc(C2CC2)c(F)c1)[C@@H]1C[C@@H](F)CN1C(=O)Cn1nnc2ccccc21] | (2S,4R)-1-[2-(1H-1,2,3- benzotriazol-1-yl)acetyl]-N-[(S)-(4- cyclopropyl-3- fluorophenyl)(phenyl)methyl]-4- fluoropyrrolidine-2-carboxamide |
| GZ-451 | 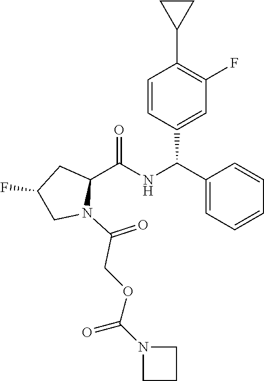 [SMILES: O=C(N[C@@H](c1ccccc1)c1ccc(C2CC2)c(F)c1)[C@@H]1C[C@@H](F)CN1C(=O)COC(=O)N1CCC1] | 2-[(2S,4R)-2-{[(S)-(4-cyclopropyl- 3- fluorophenyl)(phenyl)methyl] carbamoyl}-4-fluoropyrrolidin-1-yl]-2- oxoethyl azetidine-1-carboxylate |
| GZ-452 | 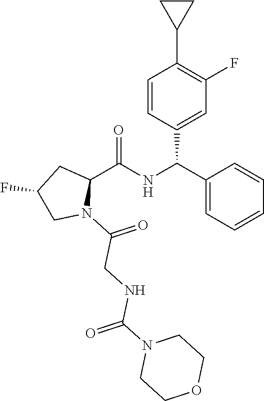 [SMILES: O=C(N[C@@H](c1ccccc1)c1ccc(C2CC2)c(F)c1)[C@@H]1C[C@@H](F)CN1C(=O)CNC(=O)N1CCOCC1] | N-{2-[(2S,4R)-2-{[(S)-(4- cyclopropyl-3- fluorophenyl)(phenyl)methyl] carbamoyl}-4-fluoropyrrolidin-1-yl]-2- oxoethyl} morpholine-4- carboxamide |
| GZ-453 | 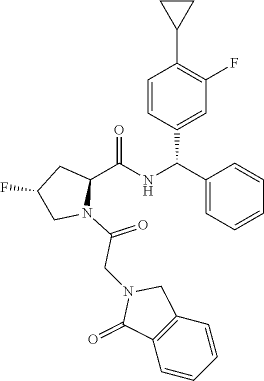 [SMILES: O=C(N[C@@H](c1ccccc1)c1ccc(C2CC2)c(F)c1)[C@@H]1C[C@@H](F)CN1C(=O)CN1Cc2ccccc2C1=O] | (2S,4R)-N-[(S)-(4-cyclopropyl-3- fluorophenyl)(phenyl)methyl]-4- fluoro-1-[2-(1-oxo-2,3-dihydro-1H- isoindol-2-yl)acetyl]pyrrolidine-2- carboxamide |
| GZ-454 | 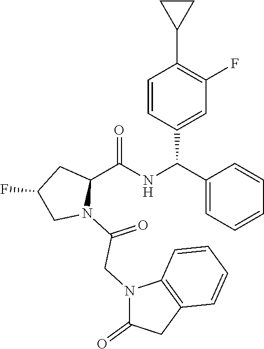 [SMILES: O=C(N[C@@H](c1ccccc1)c1ccc(C2CC2)c(F)c1)[C@@H]1C[C@@H](F)CN1C(=O)CN1C(=O)Cc2ccccc21] | (2S,4R)-N-[(S)-(4-cyclopropyl-3- fluorophenyl)(phenyl)methyl]-4- fluoro-1-[2-(2-oxo-2,3-dihydro-1H- indol-1-yl)acetyl]pyrrolidine-2- carboxamide |
| GZ-455 | 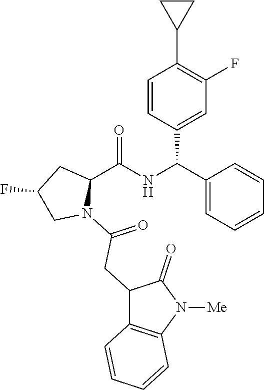 [SMILES: CN1C(=O)C(CC(=O)N2C[C@H](F)C[C@H]2C(=O)N[C@@H](c2ccccc2)c2ccc(C3CC3)c(F)c2)c2ccccc21] | (2S,4R)-N-[(S)-(4-cyclopropyl-3- fluorophenyl)(phenyl)methyl]-4- fluoro-1-[2-(1-methyl-2-oxo-2,3- dihydro-1H-indol-3- yl)acetyl]pyrrolidine-2- carboxamide |
| GZ-456 | 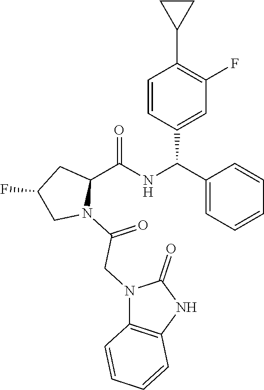 [SMILES: O=C(N[C@@H](c1ccccc1)c1ccc(C2CC2)c(F)c1)[C@@H]1C[C@@H](F)CN1C(=O)Cn1c(=O)[nH]c2ccccc21] | (2S,4R)-N-[(S)-(4-cyclopropyl-3- fluorophenyl)(phenyl)methyl]-4- fluoro-1-[2-(2-oxo-2,3-dihydro-1H- 1,3-benzodiazol-1- yl)acetyl]pyrrolidine-2- carboxamide |
| GZ-457 | 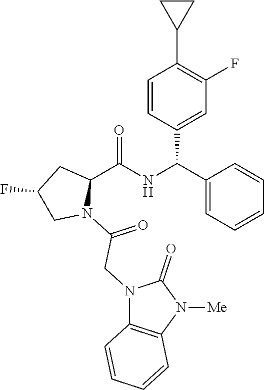 [SMILES: Cn1c(=O)n(CC(=O)N2C[C@H](F)C[C@H]2C(=O)N[C@@H](c2ccccc2)c2ccc(C3CC3)c(F)c2)c2ccccc21] | (2S,4R)-N-[(S)-(4-cyclopropyl-3- fluorophenyl)(phenyl)methyl]-4- fluoro-1-[2-(3-methyl-2-oxo-2,3- dihydro-1H-1,3-benzodiazol-1- yl)acetyl]pyrrolidine-2- carboxamide |
| GZ-458 | 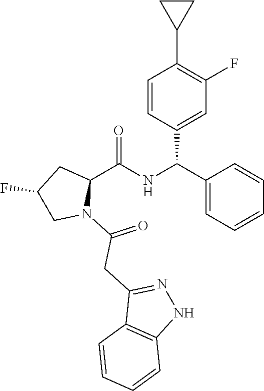 [SMILES: O=C(N[C@@H](c1ccccc1)c1ccc(C2CC2)c(F)c1)[C@@H]1C[C@@H](F)CN1C(=O)Cc1n[nH]c2ccccc12] | (2S,4R)-N-[(S)-(4-cyclopropyl-3- fluorophenyl)(phenyl)methyl]-4- fluoro-1-[2-(1H-indazol-3- yl)acetyl]pyrrolidine-2- carboxamide |
| GZ-459 | 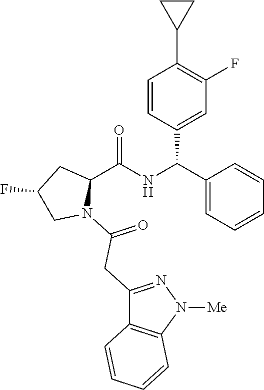 [SMILES: Cn1nc(CC(=O)N2C[C@H](F)C[C@H]2C(=O)N[C@@H](c2ccccc2)c2ccc(C3CC3)c(F)c2)c2ccccc21] | (2S,4R)-N-[(S)-(4-cyclopropyl-3- fluorophenyl)(phenyl)methyl]-4- fluoro-1-[2-(1-methyl-1H-indazol- 3-yl)acetyl]pyrrolidine-2- carboxamide |
| GZ-460 | 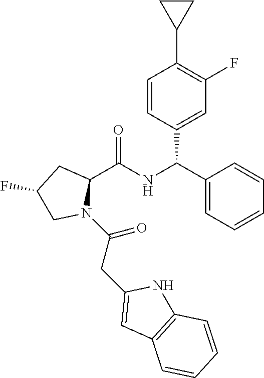 [SMILES: O=C(N[C@@H](c1ccccc1)c1ccc(C2CC2)c(F)c1)[C@@H]1C[C@@H](F)CN1C(=O)Cc1cc2ccccc2[nH]1] | (2S,4R)-N-[(S)-(4-cyclopropyl-3- fluorophenyl)(phenyl)methyl]-4- fluoro-1-[2-(1H-indol-2- yl)acetyl]pyrrolidine-2- carboxamide |
| GZ-461 | 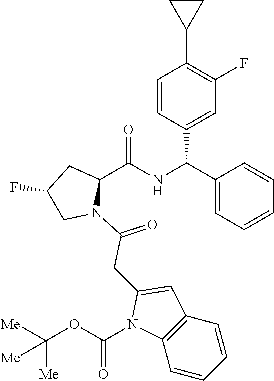 [SMILES: CC(C)(C)OC(=O)n1c(CC(=O)N2C[C@H](F)C[C@H]2C(=O)N[C@@H](c2ccccc2)c2ccc(C3CC3)c(F)c2)cc2ccccc21] | tert-butyl 2-{2-[(2S,4R)-2-{[(S)-(4- cyclopropyl-3- fluorophenyl)(phenyl)methyl] carbamoyl}-4-fluoropyrrolidin-1-yl]-2- oxoethyl}-1H-indole-1-carboxylate |
| GZ-462 | 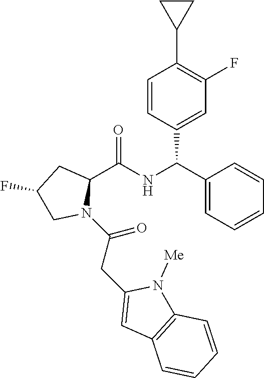 [SMILES: Cn1c(CC(=O)N2C[C@H](F)C[C@H]2C(=O)N[C@@H](c2ccccc2)c2ccc(C3CC3)c(F)c2)cc2ccccc21] | (2S,4R)-N-[(S)-(4-cyclopropyl-3- fluorophenyl)(phenyl)methyl]-4- fluoro-1-[2-(1-methyl-1H-indol-2- yl)acetyl]pyrrolidine-2- carboxamide |
| GZ-463 | 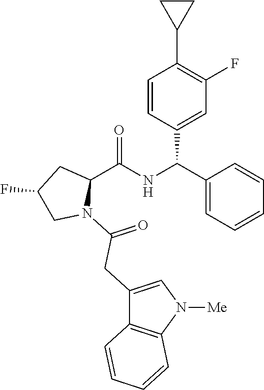 [SMILES: Cn1cc(CC(=O)N2C[C@H](F)C[C@H]2C(=O)N[C@@H](c2ccccc2)c2ccc(C3CC3)c(F)c2)c2ccccc21] | (2S,4R)-N-[(S)-(4-cyclopropyl-3- fluorophenyl)(phenyl)methyl]-4- fluoro-1-[2-(1-methyl-1H-indol-3- yl)acetyl]pyrrolidine-2- carboxamide |
| GZ-464 | 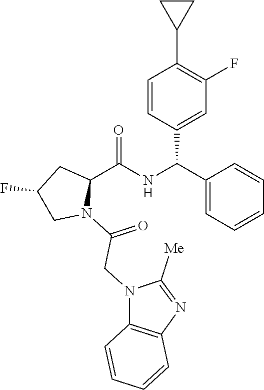 [SMILES: Cc1nc2ccccc2n1CC(=O)N1C[C@H](F)C[C@H]1C(=O)N[C@@H](c1ccccc1)c1ccc(C2CC2)c(F)c1] | (2S,4R)-N-[(S)-(4-cyclopropyl-3- fluorophenyl)(phenyl)methyl]-4- fluoro-1-[2-(2-methyl-1H-1,3- benzodiazol-1- yl)acetyl]pyrrolidine-2- carboxamide |
| GZ-465 | 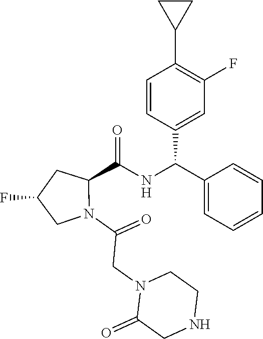 [SMILES: O=C(N[C@@H](c1ccccc1)c1ccc(C2CC2)c(F)c1)[C@@H]1C[C@@H](F)CN1C(=O)CN1CCNCC1=O] | (2S,4R)-N-[(S)-(4-cyclopropyl-3- fluorophenyl)(phenyl)methyl]-4- fluoro-1-[2-(2-oxopiperazin-1- yl)acetyl]pyrrolidine-2- carboxamide |
| GZ-466 | 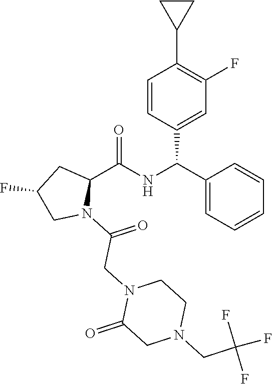 [SMILES: O=C(N[C@@H](c1ccccc1)c1ccc(C2CC2)c(F)c1)[C@@H]1C[C@@H](F)CN1C(=O)CN1CCN(CC(F)(F)F)CC1=O] | (2S,4R)-N-[(S)-(4-cyclopropyl-3- fluorophenyl)(phenyl)methyl]-4- fluoro-1-{2-[2-oxo-4-(2,2,2- trifluoroethyl)piperazin-1- yl]acetyl } pyrrolidine-2- carboxamide |
| GZ-467 | 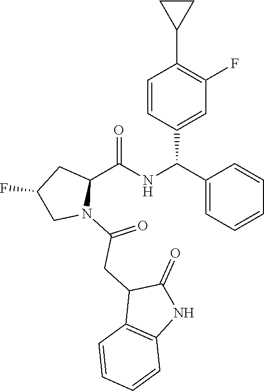 [SMILES: O=C1Nc2ccccc2C1CC(=O)N1C[C@H](F)C[C@H]1C(=O)N[C@@H](c1ccccc1)c1ccc(C2CC2)c(F)c1] | (2S,4R)-N-[(S)-(4-cyclopropyl-3- fluorophenyl)(phenyl)methyl]-4- fluoro-1-[2-(2-oxo-2,3-dihydro-1H- indol-3-yl)acetyl]pyrrolidine-2- carboxamide |
| GZ-468 | 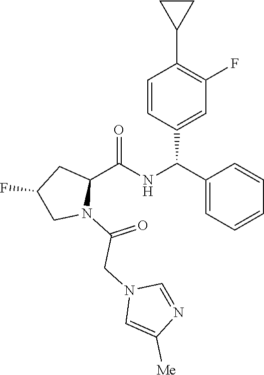 [SMILES: Cc1cn(CC(=O)N2C[C@H](F)C[C@H]2C(=O)N[C@@H](c2ccccc2)c2ccc(C3CC3)c(F)c2)cn1] | (2S,4R)-N-[(S)-(4-cyclopropyl-3- fluorophenyl)(phenyl)methyl]-4- fluoro-1-[2-(4-methyl-1H-imidazol- 1-yl)acetyl]pyrrolidine-2- carboxamide |
| GZ-469 | 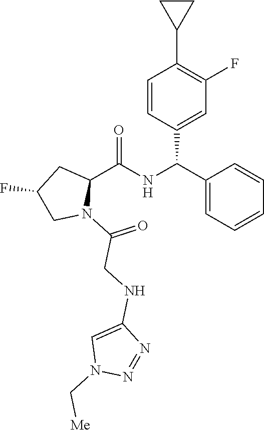 [SMILES: CCn1cc(NCC(=O)N2C[C@H](F)C[C@H]2C(=O)N[C@@H](c2ccccc2)c2ccc(C3CC3)c(F)c2)nn1] | (2S,4R)-N-[(S)-(4-cyclopropyl-3- fluorophenyl)(phenyl)methyl]-1- {2-[(1-ethyl-1H-1,2,3-triazol-4- yl)amino]acetyl}-4- fluoropyrrolidine-2-carboxamide |
| GZ-470 | 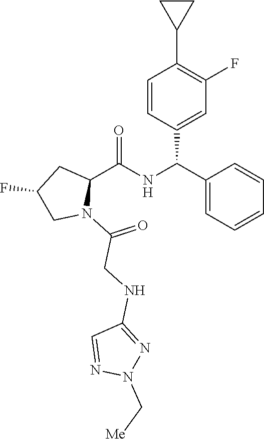 [SMILES: CCn1ncc(NCC(=O)N2C[C@H](F)C[C@H]2C(=O)N[C@@H](c2ccccc2)c2ccc(C3CC3)c(F)c2)n1] | (2S,4R)-N-[(S)-(4-cyclopropyl-3- fluorophenyl)(phenyl)methyl]-1- {2-[(2-ethyl-2H-1,2,3-triazol-4- yl)amino]acetyl}-4- fluoropyrrolidine-2-carboxamide |
| GZ-471 | 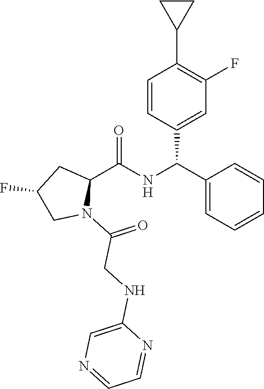 [SMILES: O=C(N[C@@H](c1ccccc1)c1ccc(C2CC2)c(F)c1)[C@@H]1C[C@@H](F)CN1C(=O)CNc1cnccn1] | (2S,4R)-N-[(S)-(4-cyclopropyl-3- fluorophenyl)(phenyl)methyl]-4- fluoro-1-{2-[(pyrazin-2- yl)amino]acetyl}pyrrolidine-2- carboxamide |
| GZ-472 | 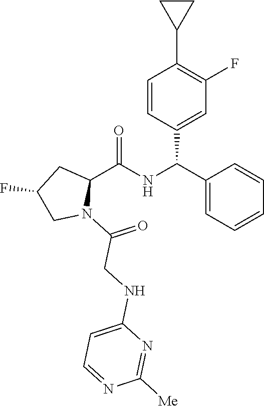 [SMILES: Cc1nccc(NCC(=O)N2C[C@H](F)C[C@H]2C(=O)N[C@@H](c2ccccc2)c2ccc(C3CC3)c(F)c2)n1] | (2S,4R)-N-[(S)-(4-cyclopropyl-3- fluorophenyl)(phenyl)methyl]-4- fluoro-1-{2-[(2-methylpyrimidin-4- yl)amino]acetyl } pyrrolidine-2- carboxamide |
| GZ-473 | 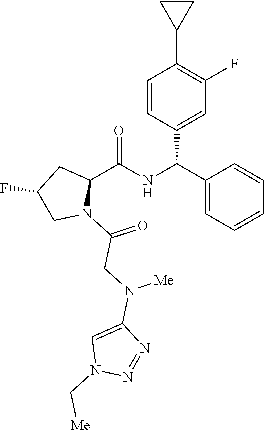 [SMILES: CCn1cc(N(C)CC(=O)N2C[C@H](F)C[C@H]2C(=O)N[C@@H](c2ccccc2)c2ccc(C3CC3)c(F)c2)nn1] | (2S,4R)-N-[(S)-(4-cyclopropyl-3- fluorophenyl)(phenyl)methyl]-1- {2-[(1-ethyl-1H-1,2,3-triazol-4- yl)(methyl)amino]acetyl}-4- fluoropyrrolidine-2-carboxamide |
| GZ-474 | 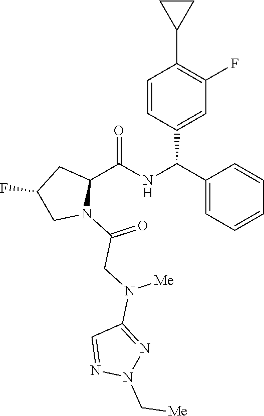 [SMILES: CCn1ncc(N(C)CC(=O)N2C[C@H](F)C[C@H]2C(=O)N[C@@H](c2ccccc2)c2ccc(C3CC3)c(F)c2)n1] | (2S,4R)-N-[(S)-(4-cyclopropyl-3- fluorophenyl)(phenyl)methyl]-1- {2-[(2-ethyl-2H-1,2,3-triazol-4- yl)(methyl)amino]acetyl}-4- fluoropyrrolidine-2-carboxamide |
| GZ-475 | 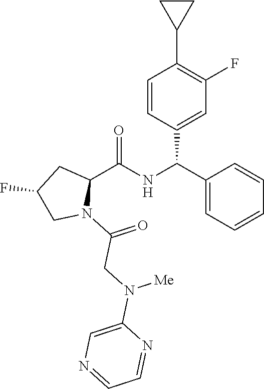 [SMILES: CN(CC(=O)N1C[C@H](F)C[C@H]1C(=O)N[C@@H](c1ccccc1)c1ccc(C2CC2)c(F)c1)c1cnccn1] | (2S,4R)-N-[(S)-(4-cyclopropyl-3- fluorophenyl)(phenyl)methyl]-4- fluoro-1-{2-[methyl(pyrazin-2- yl)amino]acetyl } pyrrolidine-2- carboxamide |
| GZ-476 | 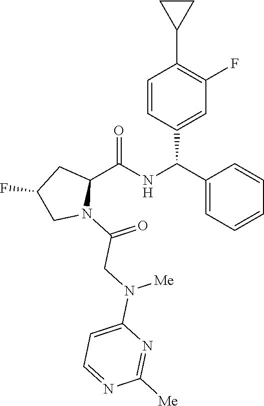 [SMILES: Cc1nccc(N(C)CC(=O)N2C[C@H](F)C[C@H]2C(=O)N[C@@H](c2ccccc2)c2ccc(C3CC3)c(F)c2)n1] | (2S,4R)-N-[(S)-(4-cyclopropyl-3- fluorophenyl)(phenyl)methyl]-4- fluoro-1-{2-[methyl(2- methylpyrimidin-4- yl)amino]acetyl } pyrrolidine-2- carboxamide |
| GZ-477 | 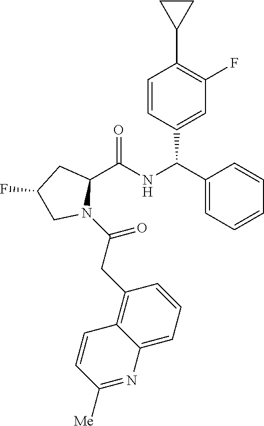 [SMILES: Cc1ccc2c(CC(=O)N3C[C@H](F)C[C@H]3C(=O)N[C@@H](c3ccccc3)c3ccc(C4CC4)c(F)c3)cccc2n1] | (2S,4R)-N-[(S)-(4-cyclopropyl-3- fluorophenyl)(phenyl)methyl]-4- fluoro-1-[2-(2-methylquinolin-5- yl)acetyl]pyrrolidine-2- carboxamide |
| GZ-478 | 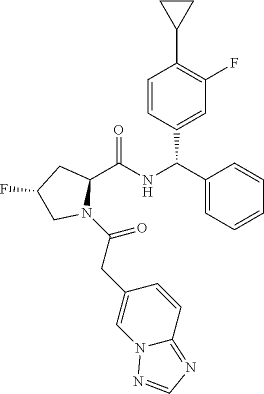 [SMILES: O=C(N[C@@H](c1ccccc1)c1ccc(C2CC2)c(F)c1)[C@@H]1C[C@@H](F)CN1C(=O)Cc1ccc2ncnn2c1] | (2S,4R)-N-[(S)-(4-cyclopropyl-3- fluorophenyl)(phenyl)methyl]-4- fluoro-1-(2-{[1,2,4]triazolo[1,5- alpyridin-6-yl} acetyl)pyrrolidine-2- carboxamide |
| GZ-479 | 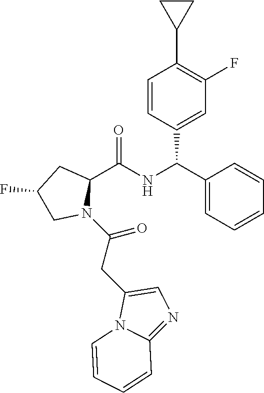 [SMILES: O=C(N[C@@H](c1ccccc1)c1ccc(C2CC2)c(F)c1)[C@@H]1C[C@@H](F)CN1C(=O)Cc1cnc2ccccn12] | (2S,4R)-N-[(S)-(4-cyclopropyl-3- fluorophenyl)(phenyl)methyl]-4- fluoro-1-(2-{imidazo[1,2-a]pyridin- 3-yl}acetyl)pyrrolidine-2- carboxamide |
| GZ-480 | 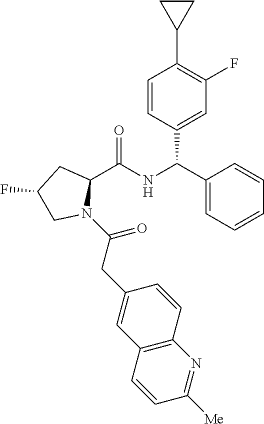 [SMILES: Cc1ccc2cc(CC(=O)N3C[C@H](F)C[C@H]3C(=O)N[C@@H](c3ccccc3)c3ccc(C4CC4)c(F)c3)ccc2n1] | (2S,4R)-N-[(S)-(4-cyclopropyl-3- fluorophenyl)(phenyl)methyl]-4- fluoro-1-[2-(2-methylquinolin-6- yl)acetyl]pyrrolidine-2- carboxamide |
| GZ-481 | 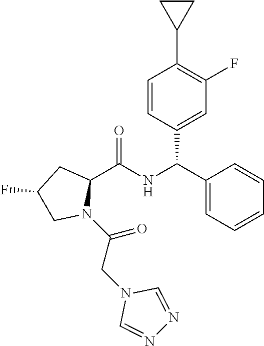 [SMILES: O=C(N[C@@H](c1ccccc1)c1ccc(C2CC2)c(F)c1)[C@@H]1C[C@@H](F)CN1C(=O)Cn1cnnc1] | (2S,4R)-N-[(S)-(4-cyclopropyl-3- fluorophenyl)(phenyl)methyl]-4- fluoro-1-[2-(4H-1,2,4-triazol-4- yl)acetyl]pyrrolidine-2- carboxamide |
| GZ-482 | 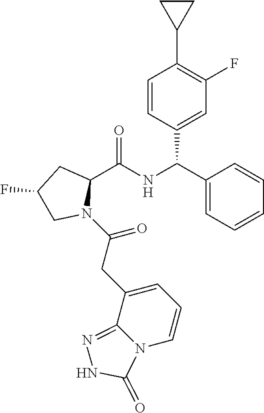 [SMILES: O=C(N[C@@H](c1ccccc1)c1ccc(C2CC2)c(F)c1)[C@@H]1C[C@@H](F)CN1C(=O)Cc1cccn2c(=O)[nH]nc12] | (2S,4R)-N-[(S)-(4-cyclopropyl-3- fluorophenyl)(phenyl)methyl]-4- fluoro-1-(2-{3-oxo-2H,3H- [1,2,4]triazolo[4,3-a]pyridin-8- yl}acetyl)pyrrolidine-2- carboxamide |
| GZ-483 | 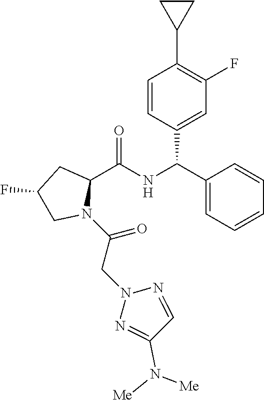 [SMILES: CN(C)c1cnn(CC(=O)N2C[C@H](F)C[C@H]2C(=O)N[C@@H](c2ccccc2)c2ccc(C3CC3)c(F)c2)n1] | (2S,4R)-N-[(S)-(4-cyclopropyl-3- fluorophenyl)(phenyl)methyl]-1- {2-[4-(dimethylamino)-2H-1,2,3- triazol-2-yl]acetyl}-4- fluoropyrrolidine-2-carboxamide |
| GZ-484 | 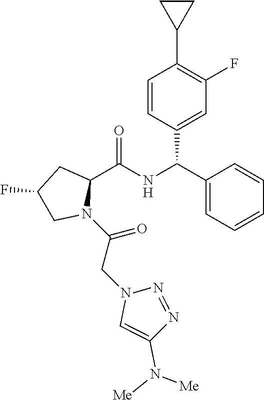 [SMILES: CN(C)c1cn(CC(=O)N2C[C@H](F)C[C@H]2C(=O)N[C@@H](c2ccccc2)c2ccc(C3CC3)c(F)c2)nn1] | (2S,4R)-N-[(S)-(4-cyclopropyl-3- fluorophenyl)(phenyl)methyl]-1- {2-[4-(dimethylamino)-1H-1,2,3- triazol-1-yl]acetyl}-4- fluoropyrrolidine-2-carboxamide |
| GZ-485 | 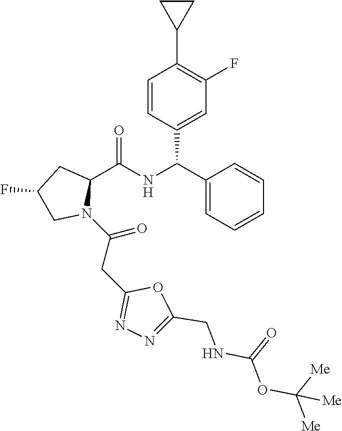 [SMILES: CC(C)(C)OC(=O)NCc1nnc(CC(=O)N2C[C@H](F)C[C@H]2C(=O)N[C@@H](c2ccccc2)c2ccc(C3CC3)c(F)c2)o1] | tert-butyl N-[(5-{2-[(2S,4R)-2- {[(S)-(4-cyclopropyl-3- fluorophenyl)(phenyl)methyl] carbamoyl}-4-fluoropyrrolidin-1-yl]-2- oxoethyl}-1,3,4-oxadiazol-2- yl)methyl]carbamate |
| GZ-486 | 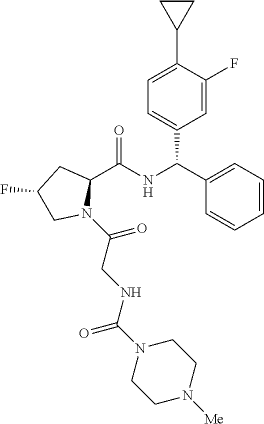 [SMILES: CN1CCN(C(=O)NCC(=O)N2C[C@H](F)C[C@H]2C(=O)N[C@@H](c2ccccc2)c2ccc(C3CC3)c(F)c2)CC1] | N-{2-[(2S,4R)-2-{[(S)-(4- cyclopropyl-3- fluorophenyl)(phenyl)methyl] carbamoyl}-4-fluoropyrrolidin-1-yl]-2- oxoethyl}-4-methylpiperazine-1- carboxamide |
| GZ-487 | 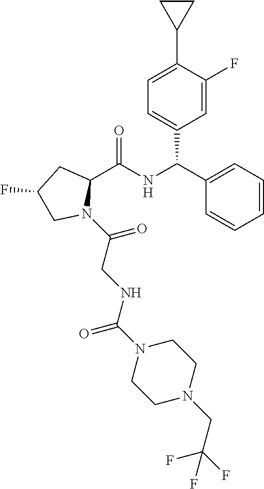 [SMILES: O=C(N[C@@H](c1ccccc1)c1ccc(C2CC2)c(F)c1)[C@@H]1C[C@@H](F)CN1C(=O)CNC(=O)N1CCN(CC(F)(F)F)CC1] | N-{2-[(2S,4R)-2-{[(S)-(4- cyclopropyl-3- fluorophenyl)(phenyl)methyl] carbamoyl}-4-fluoropyrrolidin-1-yl]-2- oxoethyl}-4-(2,2,2- trifluoroethyl)piperazine-1- carboxamide |
| GZ-488 | 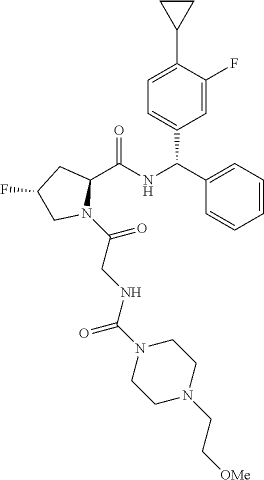 [SMILES: COCCN1CCN(C(=O)NCC(=O)N2C[C@H](F)C[C@H]2C(=O)N[C@@H](c2ccccc2)c2ccc(C3CC3)c(F)c2)CC1] | N-{2-[(2S,4R)-2-{[(S)-(4- cyclopropyl-3- fluorophenyl)(phenyl)methyl] carbamoyl}-4-fluoropyrrolidin-1-yl]-2- oxoethyl}-4-(2- methoxyethyl)piperazine-1- carboxamide |
| GZ-489 | 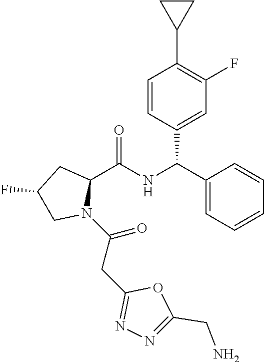 [SMILES: NCc1nnc(CC(=O)N2C[C@H](F)C[C@H]2C(=O)N[C@@H](c2ccccc2)c2ccc(C3CC3)c(F)c2)o1] | (2S,4R)-1-{2-[5-(aminomethyl)- 1,3,4-oxadiazol-2-yl]acetyl}-N- [(S)-(4-cyclopropyl-3- fluorophenyl)(phenyl)methyl]-4- fluoropyrrolidine-2-carboxamide |
| GZ-490 | 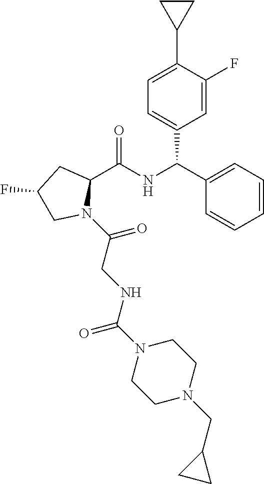 [SMILES: O=C(N[C@@H](c1ccccc1)c1ccc(C2CC2)c(F)c1)[C@@H]1C[C@@H](F)CN1C(=O)CNC(=O)N1CCN(CC2CC2)CC1] | N-{2-[(2S,4R)-2-{[(S)-(4- cyclopropyl-3- fluorophenyl)(phenyl)methyl] carbamoyl}-4-fluoropyrrolidin-1-yl]-2- oxoethyl}-4- (cyclopropylmethyl)piperazine-1- carboxamide |
| GZ-491 | 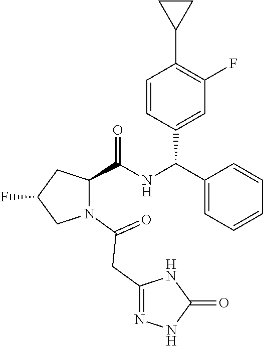 [SMILES: O=C(N[C@@H](c1ccccc1)c1ccc(C2CC2)c(F)c1)[C@@H]1C[C@@H](F)CN1C(=O)Cc1n[nH]c(=O)[nH]1] | (2S,4R)-N-[(S)-(4-cyclopropyl-3- fluorophenyl)(phenyl)methyl]-4- fluoro-1-[2-(5-oxo-4,5-dihydro-1H- 1,2,4-triazol-3- yl)acetyl]pyrrolidine-2- carboxamide |
| GZ-492 | 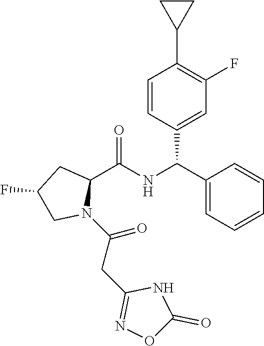 [SMILES: O=C(N[C@@H](c1ccccc1)c1ccc(C2CC2)c(F)c1)[C@@H]1C[C@@H](F)CN1C(=O)Cc1noc(=O)[nH]1] | (2S,4R)-N-[(S)-(4-cyclopropyl-3- fluorophenyl)(phenyl)methyl]-4- fluoro-1-[2-(5-oxo-4,5-dihydro- 1,2,4-oxadiazol-3- yl)acetyl]pyrrolidine-2- carboxamide |
| GZ-493 | 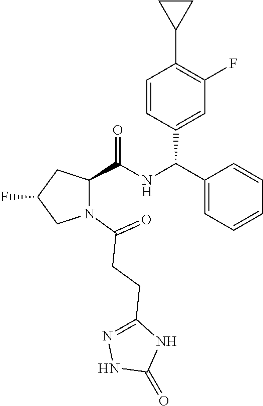 [SMILES: O=C(N[C@@H](c1ccccc1)c1ccc(C2CC2)c(F)c1)[C@@H]1C[C@@H](F)CN1C(=O)CCc1n[nH]c(=O)[nH]1] | (2S,4R)-N-[(S)-(4-cyclopropyl-3- fluorophenyl)(phenyl)methyl]-4- fluoro-1-[3-(5-oxo-4,5-dihydro-1H- 1,2,4-triazol-3- yl)propanoyl]pyrrolidine-2- carboxamide |
| GZ-494 | 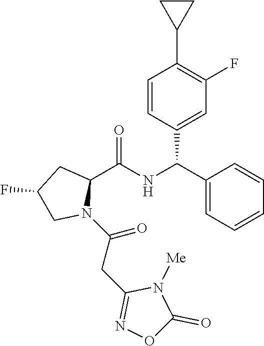 [SMILES: Cn1c(CC(=O)N2C[C@H](F)C[C@H]2C(=O)N[C@@H](c2ccccc2)c2ccc(C3CC3)c(F)c2)noc1=O] | (2S,4R)-N-[(S)-(4-cyclopropyl-3- fluorophenyl)(phenyl)methyl]-4- fluoro-1-[2-(4-methyl-5-oxo-4,5- dihydro-1,2,4-oxadiazol-3- yl)acetyl]pyrrolidine-2- carboxamide |
| GZ-495 | 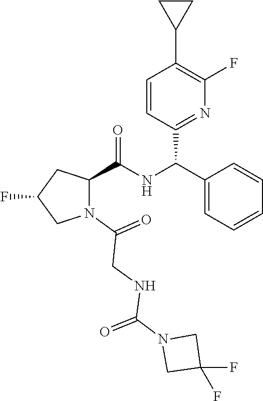 [SMILES: O=C(N[C@@H](c1ccccc1)c1ccc(C2CC2)c(F)n1)[C@@H]1C[C@@H](F)CN1C(=O)CNC(=O)N1CC(F)(F)C1] | (2S,4R)-N-[(S)-(4-cyclopropyl-3- fluorophenyl)(phenyl)methyl]-1- {2-[(3,3-difluoroazetidine-1- carbonyl)amino]acetyl}-4- fluoropyrrolidine-2-carboxamide |
| GZ-496 | 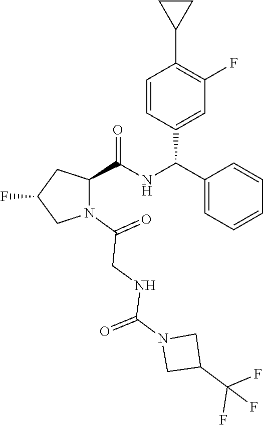 [SMILES: O=C(N[C@@H](c1ccccc1)c1ccc(C2CC2)c(F)c1)[C@@H]1C[C@@H](F)CN1C(=O)CNC(=O)N1CC(C(F)(F)F)C1] | (2S,4R)-N-[(S)-(4-cyclopropyl-3- fluorophenyl)(phenyl)methyl]-4- fluoro-1-(2-{[3- (trifluoromethyl)azetidine-1- carbonyl]amino} acetyl)pyrrolidine- 2-carboxamide |
| GZ-497 | 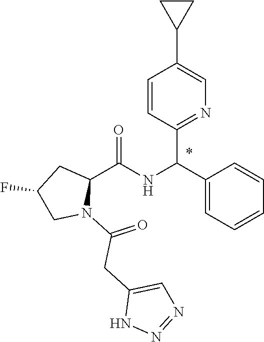 [SMILES: O=C(NC(c1ccccc1)c1ccc(C2CC2)cn1)[C@@H]1C[C@@H](F)CN1C(=O)Cc1cnn[nH]1] | (2S,4R)-1-(2-(1H-1,2,3-triazol-5- yl)acetyl)-N-((S) or (R)-(5- cyclopropylpyridin-2- yl)(phenyl)methyl)-4- fluoropyrrolidine-2-carboxamide |
| GZ-498 | 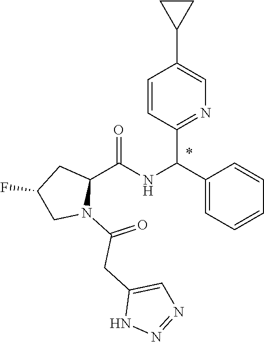 [SMILES: O=C(NC(c1ccccc1)c1ccc(C2CC2)cn1)[C@@H]1C[C@@H](F)CN1C(=O)Cc1cnn[nH]1] | (2S,4R)-1-(2-(1H-1,2,3-triazol-5- yl)acetyl)-N-((R) or (S)-(5- cyclopropylpyridin-2- yl)(phenyl)methyl)-4- fluoropyrrolidine-2-carboxamide |
| GZ-499 | 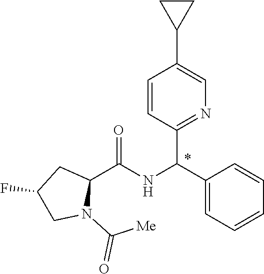 [SMILES: CC(=O)N1C[C@H](F)C[C@H]1C(=O)NC(c1ccccc1)c1ccc(C2CC2)cn1] | (2S,4R)-1-acetyl-N-[(S) or (R)-(5- cyclopropylpyridin-2- yl)(phenyl)methyl]-4- fluoropyrrolidine-2-carboxamide |
| GZ-500 | 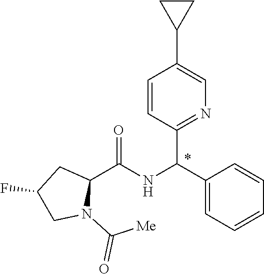 [SMILES: CC(=O)N1C[C@H](F)C[C@H]1C(=O)NC(c1ccccc1)c1ccc(C2CC2)cn1] | (2S,4R)-1-acetyl-N-[(R) or (S)-(5- cyclopropylpyridin-2- yl)(phenyl)methyl]-4- fluoropyrrolidine-2-carboxamide (second eluting isomer) |
| GZ-501 | 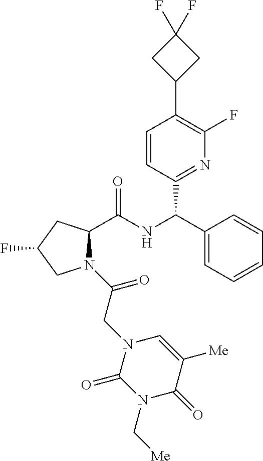 [SMILES: CCn1c(=O)c(C)cn(CC(=O)N2C[C@H](F)C[C@H]2C(=O)N[C@@H](c2ccccc2)c2ccc(C3CC(F)(F)C3)c(F)n2)c1=O] | (2S,4R)-N-((S)-(5-(3,3- difluorocyclobutyl)-6- fluoropyridin-2-yl)(phenyl)methyl)- 1-(2-(3-ethyl-5-methyl-2,4-dioxo- 3,4-dihydropyrimidin-1(2H)- yl)acetyl)-4-fluoropyrrolidine-2- carboxamide |
| GZ-502 | 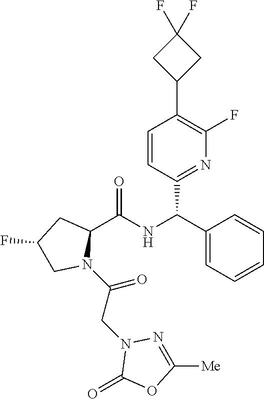 [SMILES: Cc1nn(CC(=O)N2C[C@H](F)C[C@H]2C(=O)N[C@@H](c2ccccc2)c2ccc(C3CC(F)(F)C3)c(F)n2)c(=O)o1] | (2S,4R)-N-[(S)-[5-(3,3- difluorocyclobutyl)-6- fluoropyridin-2-yl](phenyl)methyl]- 4-fluoro-1-[2-(5-methyl-2-oxo-2,3- dihydro-1,3,4-oxadiazol-3- yl)acetyl]pyrrolidine-2- carboxamide |
| GZ-503 | 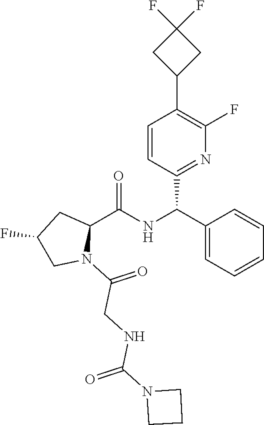 [SMILES: O=C(N[C@@H](c1ccccc1)c1ccc(C2CC(F)(F)C2)c(F)n1)[C@@H]1C[C@@H](F)CN1C(=O)CNC(=O)N1CCC1] | (2S,4R)-1-{2-[(azetidine-1- carbonyl)amino]acetyl}-N-[(S)-[5- (3,3-difluorocyclobutyl)-6- fluoropyridin-2-yl](phenyl)methyl]- 4-fluoropyrrolidine-2-carboxamide |
| GZ-504 | 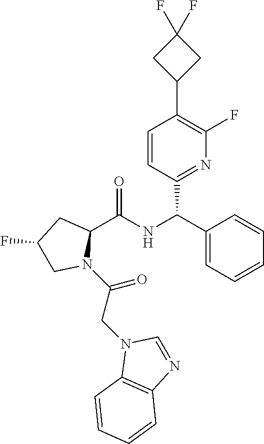 [SMILES: O=C(N[C@@H](c1ccccc1)c1ccc(C2CC(F)(F)C2)c(F)n1)[C@@H]1C[C@@H](F)CN1C(=O)Cn1cnc2ccccc21] | (2S,4R)-1-[2-(1H-1,3-benzodiazol- 1-yl)acetyl]-N-[(S)-[5-(3,3- difluorocyclobutyl)-6- fluoropyridin-2-yl](phenyl)methyl]- 4-fluoropyrrolidine-2-carboxamide |
| GZ-505 | 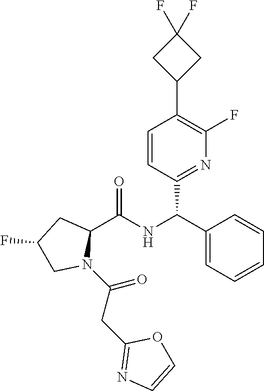 [SMILES: O=C(N[C@@H](c1ccccc1)c1ccc(C2CC(F)(F)C2)c(F)n1)[C@@H]1C[C@@H](F)CN1C(=O)Cc1ncco1] | (2S,4R)-N-[(S)-[5-(3,3- difluorocyclobutyl)-6- fluoropyridin-2-yl](phenyl)methyl]- 4-fluoro-1-[2-(1,3-oxazol-2- yl)acetyl]pyrrolidine-2- carboxamide |
| GZ-506 | 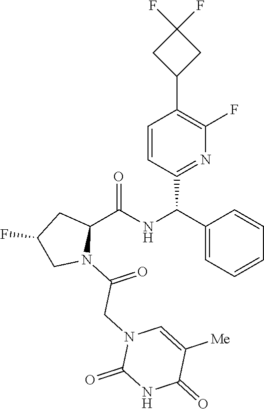 [SMILES: Cc1cn(CC(=O)N2C[C@H](F)C[C@H]2C(=O)N[C@@H](c2ccccc2)c2ccc(C3CC(F)(F)C3)c(F)n2)c(=O)[nH]c1=O] | (2S,4R)-N-[(S)-[5-(3,3- difluorocyclobutyl)-6- fluoropyridin-2-yl] (phenyl)methyl]- 4-fluoro-1-[2-(5-methyl-2,4-dioxo- 1,2,3,4-tetrahydropyrimidin-1- yl)acetyl]pyrrolidine-2- carboxamide |
| GZ-507 | 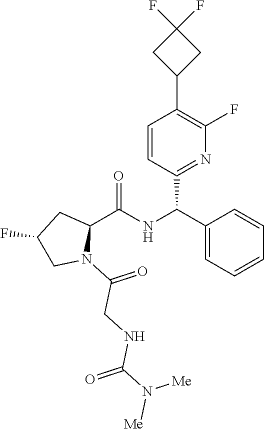 [SMILES: CN(C)C(=O)NCC(=O)N1C[C@H](F)C[C@H]1C(=O)N[C@@H](c1ccccc1)c1ccc(C2CC(F)(F)C2)c(F)n1] | (2S,4R)-N-[(S)-[5-(3,3- difluorocyclobutyl)-6- fluoropyridin-2-yl](phenyl)methyl]- 1-{2- [(dimethylcarbamoyl)amino]acetyl}- 4-fluoropyrrolidine-2-carboxamide |
| GZ-508 | 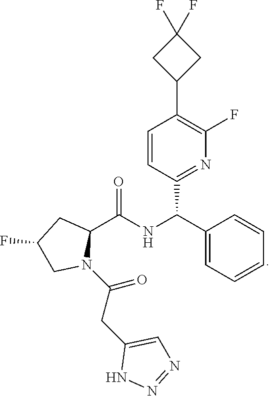 [SMILES: O=C(N[C@@H](c1ccccc1)c1ccc(C2CC(F)(F)C2)c(F)n1)[C@@H]1C[C@@H](F)CN1C(=O)Cc1cnn[nH]1] | (2S,4R)-N-[(S)-[5-(3,3- difluorocyclobutyl)-6- fluoropyridin-2-yl](phenyl)methyl]- 4-fluoro-1-[2-(1H-1,2,3-triazol-5- yl)acetyl]pyrrolidine-2- carboxamide |
| GZ-509 | 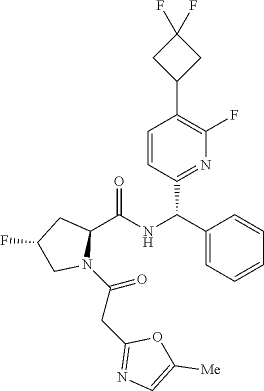 [SMILES: Cc1cnc(CC(=O)N2C[C@H](F)C[C@H]2C(=O)N[C@@H](c2ccccc2)c2ccc(C3CC(F)(F)C3)c(F)n2)o1] | (2S,4R)-N-[(S)-[5-(3,3- difluorocyclobutyl)-6- fluoropyridin-2-yl](phenyl)methyl]- 4-fluoro-1-[2-(5-methyl-1,3-oxazol- 2-yl)acetyl]pyrrolidine-2- carboxamide |
| GZ-510 | 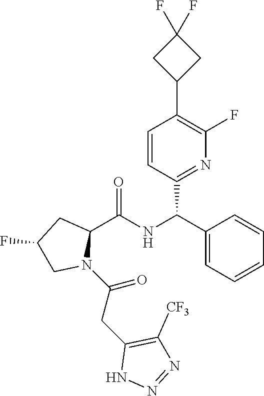 [SMILES: O=C(N[C@@H](c1ccccc1)c1ccc(C2CC(F)(F)C2)c(F)n1)[C@@H]1C[C@@H](F)CN1C(=O)Cc1[nH]nnc1C(F)(F)F] | (2S,4R)-N-[(S)-[5-(3,3- difluorocyclobutyl)-6- fluoropyridin-2-yl](phenyl)methyl]- 4-fluoro-1-{2-[4-(trifluoromethyl)- 1H-1,2,3-triazol-5- yl]acetyl} pyrrolidine-2- carboxamide |
| GZ-511 | 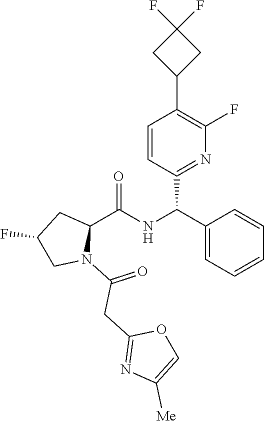 [SMILES: Cc1coc(CC(=O)N2C[C@H](F)C[C@H]2C(=O)N[C@@H](c2ccccc2)c2ccc(C3CC(F)(F)C3)c(F)n2)n1] | (2S,4R)-N-[(S)-[5-(3,3- difluorocyclobutyl)-6- fluoropyridin-2-yl](phenyl)methyl]- 4-fluoro-1-[2-(4-methyl-1,3-oxazol- 2-yl)acetyl]pyrrolidine-2- carboxamide |
| GZ-512 | 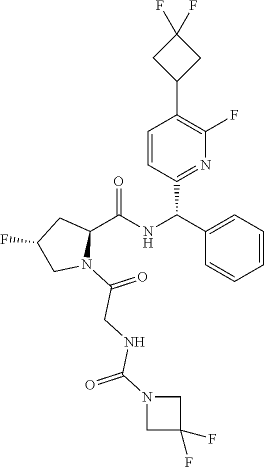 [SMILES: O=C(N[C@@H](c1ccccc1)c1ccc(C2CC(F)(F)C2)c(F)n1)[C@@H]1C[C@@H](F)CN1C(=O)CNC(=O)N1CC(F)(F)C1] | (2S,4R)-1-{2-[(3,3- difluoroazetidine-1- carbonyl)amino]acetyl}-N-[(S)-[5- (3,3-difluorocyclobutyl)-6- fluoropyridin-2-yl](phenyl)methyl]- 4-fluoropyrrolidine-2-carboxamide |
| GZ-513 | 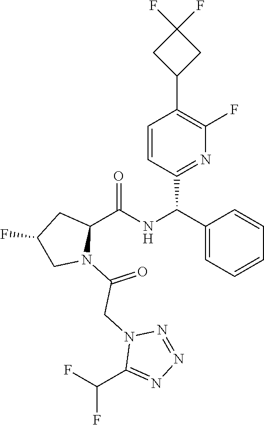 [SMILES: O=C(N[C@@H](c1ccccc1)c1ccc(C2CC(F)(F)C2)c(F)n1)[C@@H]1C[C@@H](F)CN1C(=O)Cn1nnnc1C(F)F] | (2S,4R)-N-[(S)-[5-(3,3- difluorocyclobutyl)-6- fluoropyridin-2-yl](phenyl)methyl]- 1-{2-[5-(difluoromethyl)-1H- 1,2,3,4-tetrazol-1-yl]acetyl}-4- fluoropyrrolidine-2-carboxamide |
| GZ-514 | 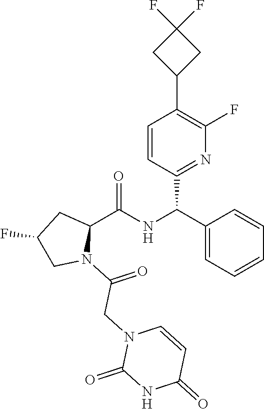 [SMILES: O=C(N[C@@H](c1ccccc1)c1ccc(C2CC(F)(F)C2)c(F)n1)[C@@H]1C[C@@H](F)CN1C(=O)Cn1ccc(=O)[nH]c1=O] | (2S,4R)-N-[(S)-[5-(3,3- difluorocyclobutyl)-6- fluoropyridin-2-yl](phenyl)methyl]- 1-[2-(2,4-dioxo-1,2,3,4- tetrahydropyrimidin-1-yl)acetyl]-4- fluoropyrrolidine-2-carboxamide |
| GZ-515 | 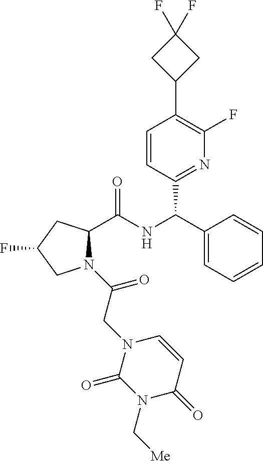 [SMILES: CCn1c(=O)ccn(CC(=O)N2C[C@H](F)C[C@H]2C(=O)N[C@@H](c2ccccc2)c2ccc(C3CC(F)(F)C3)c(F)n2)c1=O] | (2S,4R)-N-[(S)-[5-(3,3- difluorocyclobutyl)-6- fluoropyridin-2-yl](phenyl)methyl]- 1-[2-(3-ethyl-2,4-dioxo-1,2,3,4- tetrahydropyrimidin-1-yl)acetyl]-4- fluoropyrrolidine-2-carboxamide |
| GZ-516 | 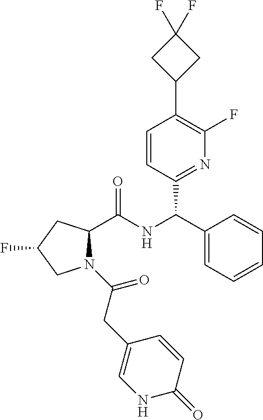 [SMILES: O=C(N[C@@H](c1ccccc1)c1ccc(C2CC(F)(F)C2)c(F)n1)[C@@H]1C[C@@H](F)CN1C(=O)Cc1ccc(=O)[nH]c1] | (2S,4R)-N-[(S)-[5-(3,3- difluorocyclobutyl)-6- fluoropyridin-2-yl](phenyl)methyl]- 4-fluoro-1-[2-(6-oxo-1,6- dihydropyridin-3- yl)acetyl]pyrrolidine-2- carboxamide |
| GZ-517 | 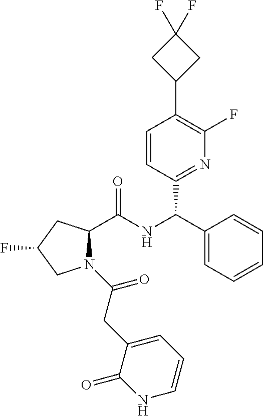 [SMILES: O=C(N[C@@H](c1ccccc1)c1ccc(C2CC(F)(F)C2)c(F)n1)[C@@H]1C[C@@H](F)CN1C(=O)Cc1ccc[nH]c1=O] | (2S,4R)-N-[(S)-[5-(3,3- difluorocyclobutyl)-6- fluoropyridin-2-yl](phenyl)methyl]- 4-fluoro-1-[2-(2-oxo-1,2- dihydropyridin-3- yl)acetyl]pyrrolidine-2- carboxamide |
| GZ-518 | 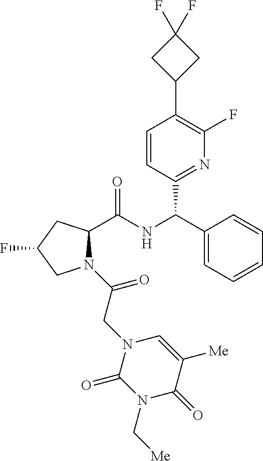 [SMILES: CCn1c(=O)c(C)cn(CC(=O)N2C[C@H](F)C[C@H]2C(=O)N[C@@H](c2ccccc2)c2ccc(C3CC(F)(F)C3)c(F)n2)c1=O] | (2S,4R)-N-[(R)-[5-(3,3- difluorocyclobutyl)-6- fluoropyridin-2-yl](phenyl)methyl]- 1-[2-(3-ethyl-5-methyl-2,4-dioxo- 1,2,3,4-tetrahydropyrimidin-1- yl)acetyl]-4-fluoropyrrolidine-2- carboxamide |
| GZ-519 | 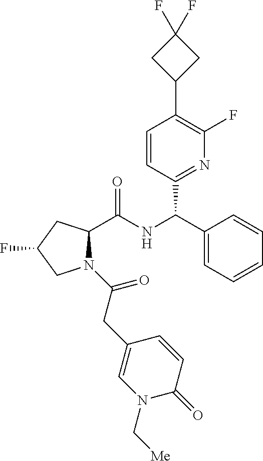 [SMILES: CCn1cc(CC(=O)N2C[C@H](F)C[C@H]2C(=O)N[C@@H](c2ccccc2)c2ccc(C3CC(F)(F)C3)c(F)n2)ccc1=O] | (2S,4R)-N-[(S)-[5-(3,3- difluorocyclobutyl)-6- fluoropyridin-2-yl](phenyl)methyl]- 1-[2-(1-ethyl-6-oxo-1,6- dihydropyridin-3-yl)acetyl]-4- fluoropyrrolidine-2-carboxamide |
| GZ-520 | 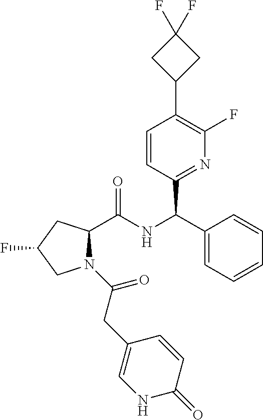 [SMILES: O=C(N[C@H](c1ccccc1)c1ccc(C2CC(F)(F)C2)c(F)n1)[C@@H]1C[C@@H](F)CN1C(=O)Cc1ccc(=O)[nH]c1] | (2S,4R)-N-[(R)-[5-(3,3- difluorocyclobutyl)-6- fluoropyridin-2-yl](phenyl)methyl]- 4-fluoro-1-[2-(6-oxo-1,6- dihydropyridin-3- yl)acetyl]pyrrolidine-2- carboxamide |
| GZ-521 | 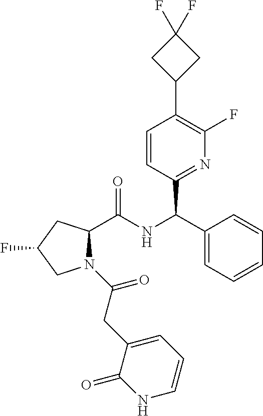 [SMILES: O=C(N[C@H](c1ccccc1)c1ccc(C2CC(F)(F)C2)c(F)n1)[C@@H]1C[C@@H](F)CN1C(=O)Cc1ccc[nH]c1=O] | (2S,4R)-N-[(R)-[5-(3,3- difluorocyclobutyl)-6- fluoropyridin-2-yl](phenyl)methyl]- 4-fluoro-1-[2-(2-oxo-1,2- dihydropyridin-3- yl)acetyl]pyrrolidine-2- carboxamide |
| GZ-522 | 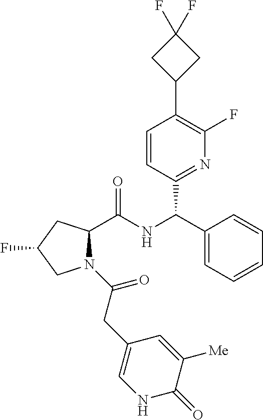 [SMILES: Cc1cc(CC(=O)N2C[C@H](F)C[C@H]2C(=O)N[C@@H](c2ccccc2)c2ccc(C3CC(F)(F)C3)c(F)n2)c[nH]c1=O] | (2S,4R)-N-[(S)-[5-(3,3- difluorocyclobutyl)-6- fluoropyridin-2-yl](phenyl)methyl]- 4-fluoro-1-[2-(5-methyl-6-oxo-1,6- dihydropyridin-3- yl)acetyl]pyrrolidine-2- carboxamide |
| GZ-523 | 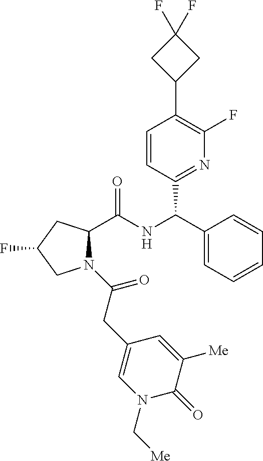 [SMILES: CCn1cc(CC(=O)N2C[C@H](F)C[C@H]2C(=O)N[C@@H](c2ccccc2)c2ccc(C3CC(F)(F)C3)c(F)n2)cc(C)c1=O] | (2S,4R)-N-[(S)-[5-(3,3- difluorocyclobutyl)-6- fluoropyridin-2-yl](phenyl)methyl]- 1-[2-(1-ethyl-5-methyl-6-oxo-1,6- dihydropyridin-3-yl)acetyl]-4- fluoropyrrolidine-2-carboxamide |
| GZ-524 | 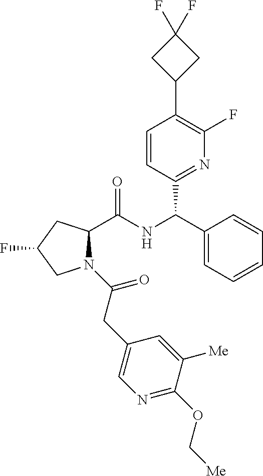 [SMILES: CCOc1ncc(CC(=O)N2C[C@H](F)C[C@H]2C(=O)N[C@@H](c2ccccc2)c2ccc(C3CC(F)(F)C3)c(F)n2)cc1C] | (2S,4R)-N-[(S)-[5-(3,3- difluorocyclobutyl)-6- fluoropyridin-2-yl](phenyl)methyl]- 1-[2-(6-ethoxy-5-methylpyridin-3- yl)acetyl]-4-fluoropyrrolidine-2- carboxamide |
| GZ-525 | 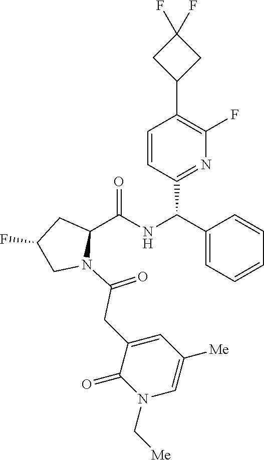 [SMILES: CCn1cc(C)cc(CC(=O)N2C[C@H](F)C[C@H]2C(=O)N[C@@H](c2ccccc2)c2ccc(C3CC(F)(F)C3)c(F)n2)c1=O] | (2S,4R)-N-[(S)-[5-(3,3- difluorocyclobutyl)-6- fluoropyridin-2-yl](phenyl)methyl]- 1-[2-(1-ethyl-5-methyl-2-oxo-1,2- dihydropyridin-3-yl)acetyl]-4- fluoropyrrolidine-2-carboxamide |
| GZ-526 | 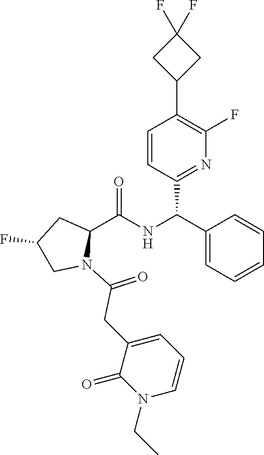 [SMILES: CCn1cccc(CC(=O)N2C[C@H](F)C[C@H]2C(=O)N[C@@H](c2ccccc2)c2ccc(C3CC(F)(F)C3)c(F)n2)c1=O] | (2S,4R)-N-[(S)-[5-(3,3- difluorocyclobutyl)-6- fluoropyridin-2-yl](phenyl)methyl]- 1-[2-(1-ethyl-2-oxo-1,2- dihydropyridin-3-yl)acetyl]-4- fluoropyrrolidine-2-carboxamide |
| GZ-527 | 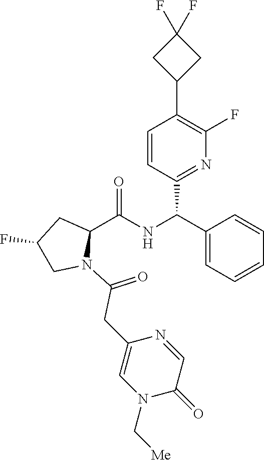 [SMILES: CCn1cc(CC(=O)N2C[C@H](F)C[C@H]2C(=O)N[C@@H](c2ccccc2)c2ccc(C3CC(F)(F)C3)c(F)n2)ncc1=O] | (2S,4R)-N-[(S)-[5-(3,3- difluorocyclobutyl)-6- fluoropyridin-2-yl](phenyl)methyl]- 1-[2-(4-ethyl-5-oxo-4,5- dihydropyrazin-2-yl)acetyl]-4- fluoropyrrolidine-2-carboxamide |
| GZ-528 | 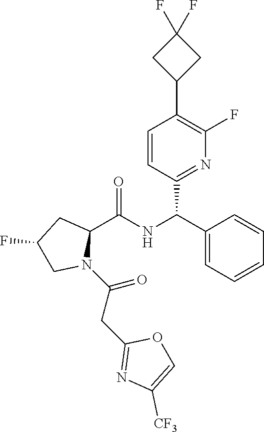 [SMILES: O=C(N[C@@H](c1ccccc1)c1ccc(C2CC(F)(F)C2)c(F)n1)[C@@H]1C[C@@H](F)CN1C(=O)Cc1nc(C(F)(F)F)co1] | (2S,4R)-N-[(S)-[5-(3,3- difluorocyclobutyl)-6- fluoropyridin-2-yl](phenyl)methyl]- 4-fluoro-1-{2-[4-(trifluoromethyl)- 1,3-oxazol-2-yl]acetyl } pyrrolidine- 2-carboxamide |
| GZ-529 | 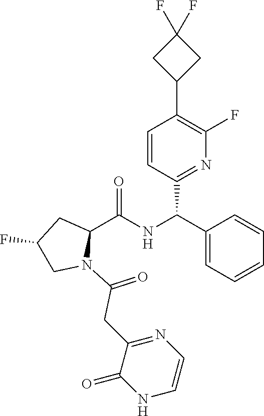 [SMILES: O=C(N[C@@H](c1ccccc1)c1ccc(C2CC(F)(F)C2)c(F)n1)[C@@H]1C[C@@H](F)CN1C(=O)Cc1ncc[nH]c1=O] | (2S,4R)-N-[(S)-[5-(3,3- difluorocyclobutyl)-6- fluoropyridin-2-yl](phenyl)methyl]- 4-fluoro-1-[2-(3-oxo-3,4- dihydropyrazin-2- yl)acetyl]pyrrolidine-2- carboxamide |
| GZ-530 | 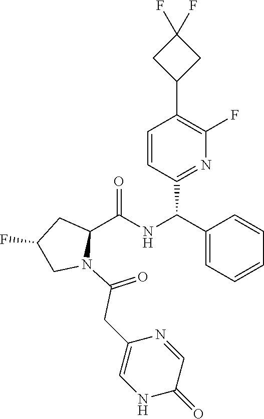 [SMILES: O=C(N[C@@H](c1ccccc1)c1ccc(C2CC(F)(F)C2)c(F)n1)[C@@H]1C[C@@H](F)CN1C(=O)Cc1c[nH]c(=O)cn1] | (2S,4R)-N-[(S)-[5-(3,3- difluorocyclobutyl)-6- fluoropyridin-2-yl](phenyl)methyl]- 4-fluoro-1-[2-(5-oxo-4,5- dihydropyrazin-2- yl)acetyl]pyrrolidine-2- carboxamide |
| GZ-531 | 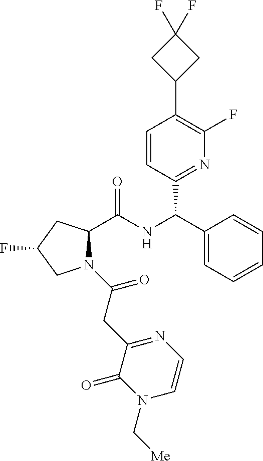 [SMILES: CCn1ccnc(CC(=O)N2C[C@H](F)C[C@H]2C(=O)N[C@@H](c2ccccc2)c2ccc(C3CC(F)(F)C3)c(F)n2)c1=O] | (2S,4R)-N-[(S)-[5-(3,3- difluorocyclobutyl)-6- fluoropyridin-2-yl](phenyl)methyl]- 1-[2-(4-ethyl-3-oxo-3,4- dihydropyrazin-2-yl)acetyl]-4- fluoropyrrolidine-2-carboxamide |
| GZ-532 | 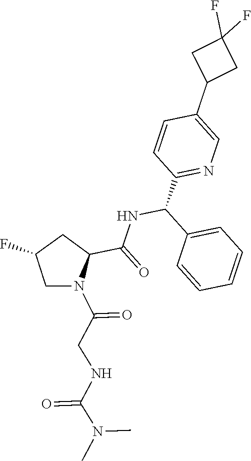 [SMILES: CN(C)C(=O)NCC(=O)N1C[C@H](F)C[C@H]1C(=O)N[C@@H](c1ccccc1)c1ccc(C2CC(F)(F)C2)cn1] | (2S,4R)-N-((S)-(5-(3,3- difluorocyclobutyl)pyridin-2- yl)(phenyl)methyl)-1- ((dimethylcarbamoyl)glycyl)-4- fluoropyrrolidine-2-carboxamide |
| GZ-533 | 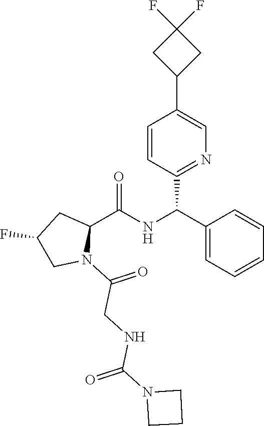 [SMILES: O=C(N[C@@H](c1ccccc1)c1ccc(C2CC(F)(F)C2)cn1)[C@@H]1C[C@@H](F)CN1C(=O)CNC(=O)N1CCC1] | (2S,4R)-1-{2-[(azetidine-1- carbonyl)amino]acetyl}-N-[(S)-[5- (3,3-difluorocyclobutyl)pyridin-2- yl](phenyl)methyl]-4- fluoropyrrolidine-2-carboxamide |
| GZ-534 | 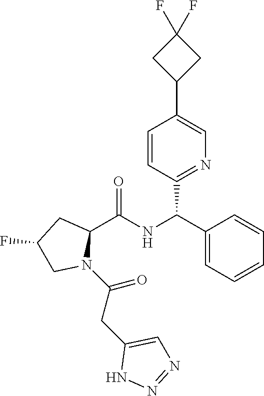 [SMILES: O=C(N[C@@H](c1ccccc1)c1ccc(C2CC(F)(F)C2)cn1)[C@@H]1C[C@@H](F)CN1C(=O)Cc1cnn[nH]1] | (2S,4R)-N-[(S)-[5-(3,3- difluorocyclobutyl)pyridin-2- yl](phenyl)methyl]-4-fluoro-1-[2- (1H-1,2,3-triazol-5- yl)acetyl]pyrrolidine-2- carboxamide |
| GZ-535 | 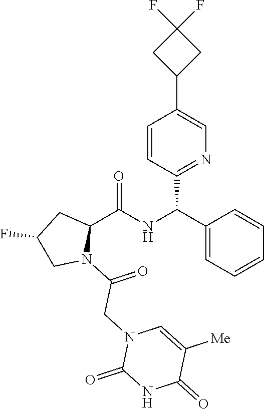 [SMILES: Cc1cn(CC(=O)N2C[C@H](F)C[C@H]2C(=O)N[C@@H](c2ccccc2)c2ccc(C3CC(F)(F)C3)cn2)c(=O)[nH]c1=O] | (2S,4R)-N-[(S)-[5-(3,3- difluorocyclobutyl)pyridin-2- yl](phenyl)methyl]-4-fluoro-1-[2- (5-methyl-2,4-dioxo-1,2,3,4- tetrahydropyrimidin-1- yl)acetyl]pyrrolidine-2- carboxamide |
| GZ-536 | 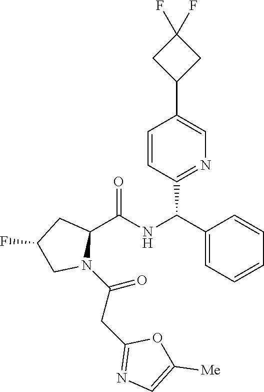 [SMILES: Cc1cnc(CC(=O)N2C[C@H](F)C[C@H]2C(=O)N[C@@H](c2ccccc2)c2ccc(C3CC(F)(F)C3)cn2)o1] | (2S,4R)-N-[(S)-[5-(3,3- difluorocyclobutyl)pyridin-2- yl](phenyl)methyl]-4-fluoro-1-[2- (5-methyl-1,3-oxazol-2- yl)acetyl]pyrrolidine-2- carboxamide |
| GZ-537 | 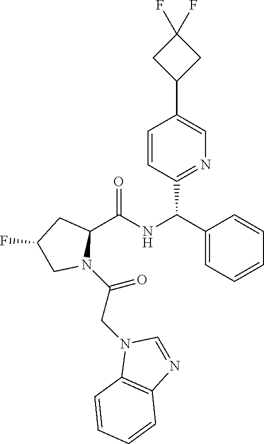 [SMILES: O=C(N[C@@H](c1ccccc1)c1ccc(C2CC(F)(F)C2)cn1)[C@@H]1C[C@@H](F)CN1C(=O)Cn1cnc2ccccc21] | (2S,4R)-1-[2-(1H-1,3-benzodiazol- 1-yl)acetyl]-N-[(S)-[5-(3,3- difluorocyclobutyl)pyridin-2- yl](phenyl)methyl]-4- fluoropyrrolidine-2-carboxamide |
| GZ-538 | 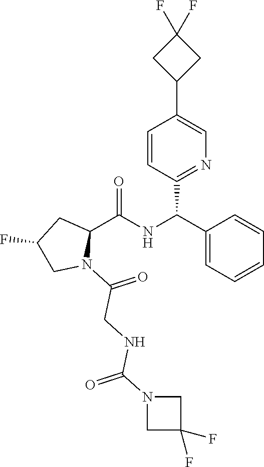 [SMILES: O=C(N[C@@H](c1ccccc1)c1ccc(C2CC(F)(F)C2)cn1)[C@@H]1C[C@@H](F)CN1C(=O)CNC(=O)N1CC(F)(F)C1] | (2S,4R)-1-{2-[(3,3- difluoroazetidine-1- carbonyl)amino]acetyl}-N-[(S)-[5- (3,3-difluorocyclobutyl)pyridin-2- yl](phenyl)methyl]-4- fluoropyrrolidine-2-carboxamide |
| GZ-539 | 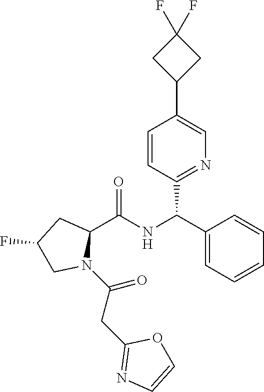 [SMILES: O=C(N[C@@H](c1ccccc1)c1ccc(C2CC(F)(F)C2)cn1)[C@@H]1C[C@@H](F)CN1C(=O)Cc1ncco1] | (2S,4R)-N-[(S)-[5-(3,3- difluorocyclobutyl)pyridin-2- yl](phenyl)methyl]-4-fluoro-1-[2- (1,3-oxazol-2-yl)acetyl]pyrrolidine- 2-carboxamide |
| GZ-540 | 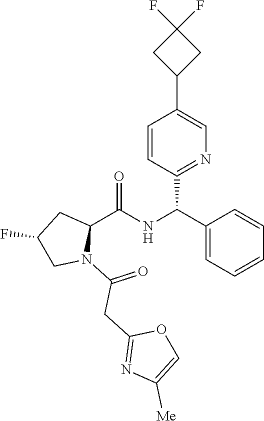 [SMILES: Cc1coc(CC(=O)N2C[C@H](F)C[C@H]2C(=O)N[C@@H](c2ccccc2)c2ccc(C3CC(F)(F)C3)cn2)n1] | (2S,4R)-N-[(S)-[5-(3,3- difluorocyclobutyl)pyridin-2- yl](phenyl)methyl]-4-fluoro-1-[2- (4-methyl-1,3-oxazol-2- yl)acetyl]pyrrolidine-2- carboxamide |
| GZ-541 | 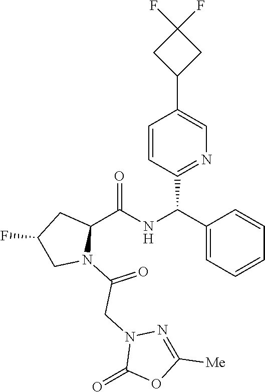 [SMILES: Cc1nn(CC(=O)N2C[C@H](F)C[C@H]2C(=O)N[C@@H](c2ccccc2)c2ccc(C3CC(F)(F)C3)cn2)c(=O)o1] | (2S,4R)-N-[(S)-[5-(3,3- difluorocyclobutyl)pyridin-2- yl](phenyl)methyl]-4-fluoro-1-[2- (5-methyl-2-oxo-2,3-dihydro-1,3,4- oxadiazol-3-yl)acetyl]pyrrolidine- 2-carboxamide |
| GZ-542 | 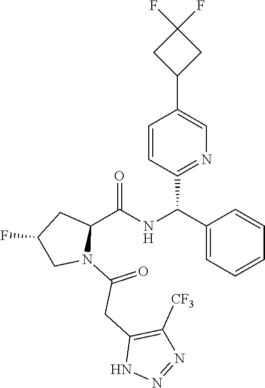 [SMILES: O=C(N[C@@H](c1ccccc1)c1ccc(C2CC(F)(F)C2)cn1)[C@@H]1C[C@@H](F)CN1C(=O)Cc1[nH]nnc1C(F)(F)F] | (2S,4R)-N-[(S)-[5-(3,3- difluorocyclobutyl)pyridin-2- yl](phenyl)methyl]-4-fluoro-1-{2- [4-(trifluoromethyl)-1H-1,2,3- triazol-5-yl]acetyl } pyrrolidine-2- carboxamide |
| GZ-543 | 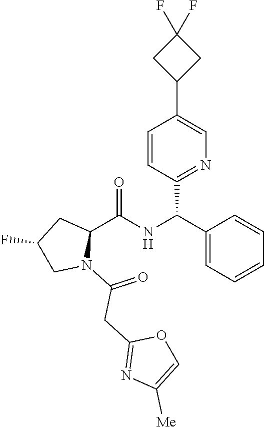 [SMILES: Cc1coc(CC(=O)N2C[C@H](F)C[C@H]2C(=O)N[C@@H](c2ccccc2)c2ccc(C3CC(F)(F)C3)cn2)n1] | (2S,4R)-N-[(S)-[5-(3,3- difluorocyclobutyl)pyridin-2- yl](phenyl)methyl]-4-fluoro-1-{2- [4-(trifluoromethyl)-1,3-oxazol-2- yl]acetyl} pyrrolidine-2- carboxamide |
| GZ-544 | 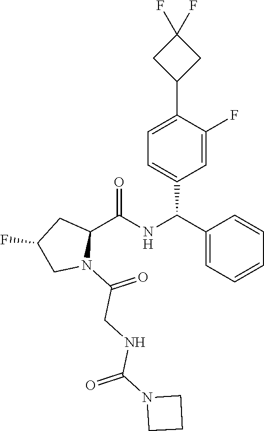 [SMILES: O=C(N[C@@H](c1ccccc1)c1ccc(C2CC(F)(F)C2)c(F)c1)[C@@H]1C[C@@H](F)CN1C(=O)CNC(=O)N1CCC1] | (2S,4R)-1-((azetidine-1- carbonyl)glycyl)-N-((S)-(4-(3,3- difluorocyclobutyl)-3- fluorophenyl)(phenyl)methyl)-4- fluoropyrrolidine-2-carboxamide |
| GZ-545 | 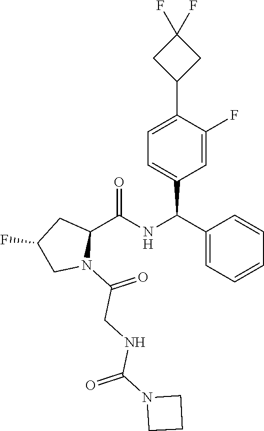 [SMILES: O=C(N[C@H](c1ccccc1)c1ccc(C2CC(F)(F)C2)c(F)c1)[C@@H]1C[C@@H](F)CN1C(=O)CNC(=O)N1CCC1] | (2S,4R)-1-((azetidine-1- carbonyl)glycyl)-N-((R)-(4-(3,3- difluorocyclobutyl)-3- fluorophenyl)(phenyl)methyl)-4- fluoropyrrolidine-2-carboxamide |
| GZ-546 | 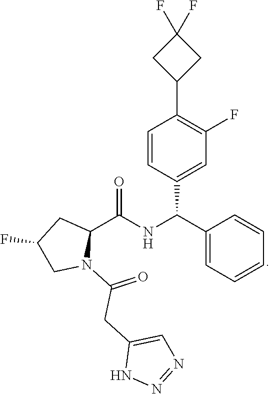 [SMILES: O=C(N[C@@H](c1ccccc1)c1ccc(C2CC(F)(F)C2)c(F)c1)[C@@H]1C[C@@H](F)CN1C(=O)Cc1cnn[nH]1] | (2S,4R)-N-[(S)-[4-(3,3- difluorocyclobutyl)-3- fluorophenyl](phenyl)methyl]-4- fluoro-1-[2-(1H-1,2,3-triazol-5- yl)acetyl]pyrrolidine-2- carboxamide |
| GZ-547 | 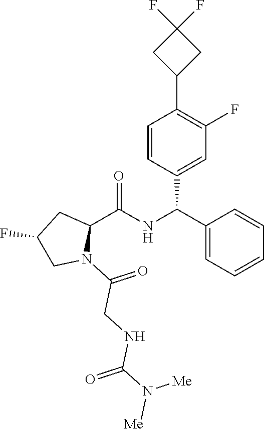 [SMILES: CN(C)C(=O)NCC(=O)N1C[C@H](F)C[C@H]1C(=O)N[C@@H](c1ccccc1)c1ccc(C2CC(F)(F)C2)c(F)c1] | (2S,4R)-N-[(S)-[4-(3,3- difluorocyclobutyl)-3- fluorophenyl](phenyl)methyl]-1- {2- [(dimethylcarbamoyl)amino]acetyl}- 4-fluoropyrrolidine-2-carboxamide |
| GZ-548 | 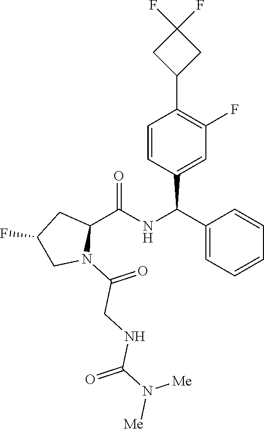 [SMILES: CN(C)C(=O)NCC(=O)N1C[C@H](F)C[C@H]1C(=O)N[C@H](c1ccccc1)c1ccc(C2CC(F)(F)C2)c(F)c1] | (2S,4R)-N-[(R)-[4-(3,3- difluorocyclobutyl)-3- fluorophenyl](phenyl)methyl]-1- {2- [(dimethylcarbamoyl)amino]acetyl}- 4-fluoropyrrolidine-2-carboxamide |
| GZ-549 | 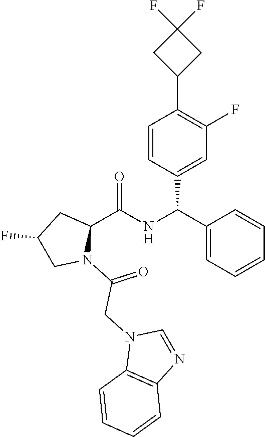 [SMILES: O=C(N[C@@H](c1ccccc1)c1ccc(C2CC(F)(F)C2)c(F)c1)[C@@H]1C[C@@H](F)CN1C(=O)Cn1cnc2ccccc21] | (2S,4R)-1-[2-(1H-1,3-benzodiazol- 1-yl)acetyl]-N-[(S)-[4-(3,3- difluorocyclobutyl)-3- fluorophenyl](phenyl)methyl]-4- fluoropyrrolidine-2-carboxamide |
| GZ-550 | 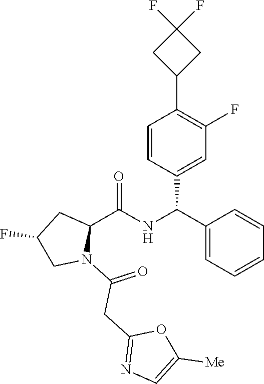 [SMILES: Cc1cnc(CC(=O)N2C[C@H](F)C[C@H]2C(=O)N[C@@H](c2ccccc2)c2ccc(C3CC(F)(F)C3)c(F)c2)o1] | (2S,4R)-N-[(S)-[4-(3,3- difluorocyclobutyl)-3- fluorophenyl](phenyl)methyl]-4- fluoro-1-[2-(5-methyl-1,3-oxazol-2- yl)acetyl]pyrrolidine-2- carboxamide |
| GZ-551 | 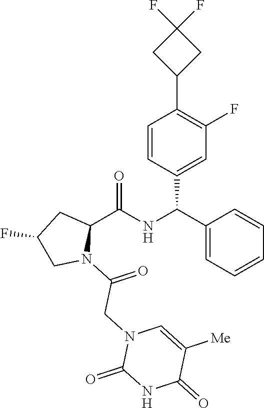 [SMILES: Cc1cn(CC(=O)N2C[C@H](F)C[C@H]2C(=O)N[C@@H](c2ccccc2)c2ccc(C3CC(F)(F)C3)c(F)c2)c(=O)[nH]c1=O] | (2S,4R)-N-[(S)-[4-(3,3- difluorocyclobutyl)-3- fluorophenyl](phenyl)methyl]-4- fluoro-1-[2-(5-methyl-2,4-dioxo- 1,2,3,4-tetrahydropyrimidin-1- yl)acetyl]pyrrolidine-2- carboxamide |
| GZ-552 | 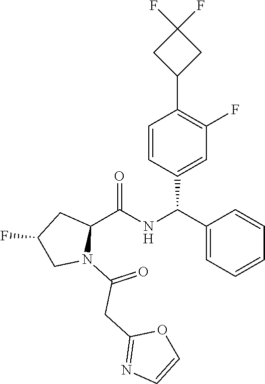 [SMILES: O=C(N[C@@H](c1ccccc1)c1ccc(C2CC(F)(F)C2)c(F)c1)[C@@H]1C[C@@H](F)CN1C(=O)Cc1ncco1] | (2S,4R)-N-[(S)-[4-(3,3- difluorocyclobutyl)-3- fluorophenyl](phenyl)methyl]-4- fluoro-1-[2-(1,3-oxazol-2- yl)acetyl]pyrrolidine-2- carboxamide |
| GZ-553 | 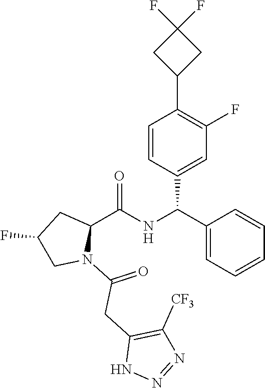 [SMILES: O=C(N[C@@H](c1ccccc1)c1ccc(C2CC(F)(F)C2)c(F)c1)[C@@H]1C[C@@H](F)CN1C(=O)Cc1[nH]nnc1C(F)(F)F] | (2S,4R)-N-[(S)-[4-(3,3- difluorocyclobutyl)-3- fluorophenyl](phenyl)methyl]-4- fluoro-1-{2-[4-(trifluoromethyl)- 1H-1,2,3-triazol-5- yl]acetyl} pyrrolidine-2- carboxamide |
| GZ-554 | 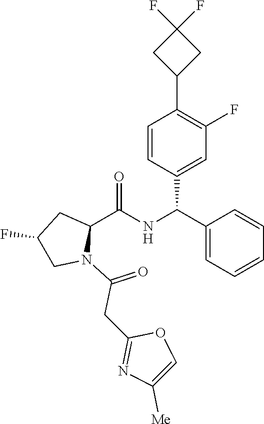 [SMILES: Cc1coc(CC(=O)N2C[C@H](F)C[C@H]2C(=O)N[C@@H](c2ccccc2)c2ccc(C3CC(F)(F)C3)c(F)c2)n1] | (2S,4R)-N-[(S)-[4-(3,3- difluorocyclobutyl)-3- fluorophenyl](phenyl)methyl]-4- fluoro-1-[2-(4-methyl-1,3-oxazol-2- yl)acetyl]pyrrolidine-2- carboxamide |
| GZ-555 | 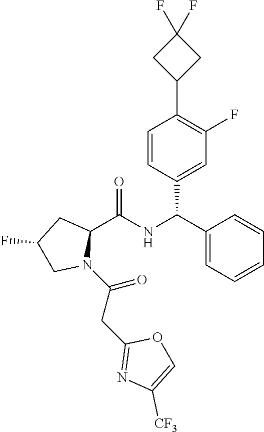 [SMILES: O=C(N[C@@H](c1ccccc1)c1ccc(C2CC(F)(F)C2)c(F)c1)[C@@H]1C[C@@H](F)CN1C(=O)Cc1nc(C(F)(F)F)co1] | (2S,4R)-N-[(S)-[4-(3,3- difluorocyclobutyl)-3- fluorophenyl](phenyl)methyl]-4- fluoro-1-{2-[4-(trifluoromethyl)- 1,3-oxazol-2-yl]acetyl} pyrrolidine- 2-carboxamide |
| GZ-556 | 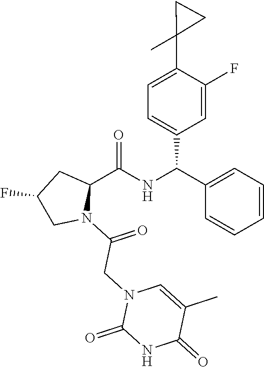 [SMILES: Cc1cn(CC(=O)N2C[C@H](F)C[C@H]2C(=O)N[C@@H](c2ccccc2)c2ccc(C3(C)CC3)c(F)c2)c(=O)[nH]c1=O] | (2S,4R)-4-fluoro-N-((S)-(3-fluoro- 4-(1- methylcyclopropyl)phenyl)(phenyl) methyl)-1-(2-(5-methyl-2,4-dioxo- 3,4-dihydropyrimidin-1(2H)- yl)acetyl)pyrrolidine-2- carboxamide |
| GZ-557 | 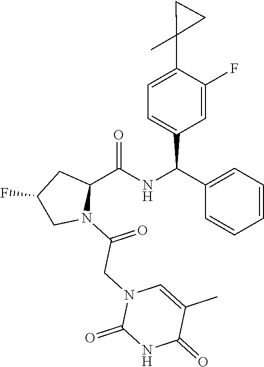 [SMILES: Cc1cn(CC(=O)N2C[C@H](F)C[C@H]2C(=O)N[C@H](c2ccccc2)c2ccc(C3(C)CC3)c(F)c2)c(=O)[nH]c1=O] | (2S,4R)-4-fluoro-N-((R)-(3-fluoro- 4-(1- methylcyclopropyl)phenyl)(phenyl) methyl)-1-(2-(5-methyl-2,4-dioxo- 3,4-dihydropyrimidin-1(2H)- yl)acetyl)pyrrolidine-2- carboxamide |
| GZ-558 | 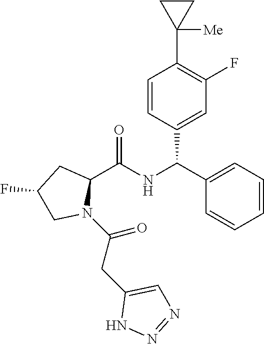 [SMILES: CC1(c2ccc([C@@H](NC(=O)[C@@H]3C[C@@H](F)CN3C(=O)Cc3cnn[nH]3)c3ccccc3)cc2F)CC1] | (2S,4R)-4-fluoro-N-[(S)-[3-fluoro- 4-(1- methylcyclopropyl)phenyl](phenyl) methyl]-1-[2-(1H-1,2,3-triazol-5- yl)acetyl]pyrrolidine-2- carboxamide |
| GZ-559 | 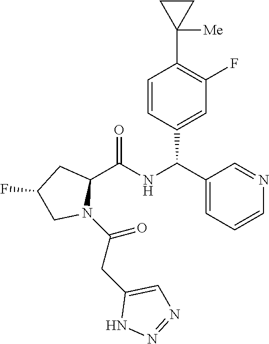 [SMILES: CC1(c2ccc([C@@H](NC(=O)[C@@H]3C[C@@H](F)CN3C(=O)Cc3cnn[nH]3)c3cccnc3)cc2F)CC1] | (2S,4R)-4-fluoro-N-[(R)-[3-fluoro- 4-(1- methylcyclopropyl)phenyl] (pyridin- 3-yl)methyl]-1-[2-(1H-1,2,3- triazol-5-yl)acetyl]pyrrolidine-2- carboxamide |
| GZ-560 | 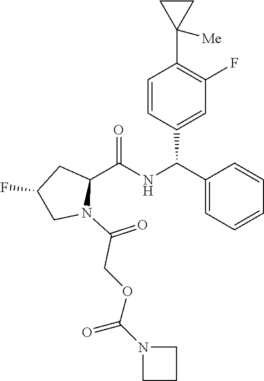 [SMILES: CC1(c2ccc([C@@H](NC(=O)[C@@H]3C[C@@H](F)CN3C(=O)COC(=O)N3CCC3)c3ccccc3)cc2F)CC1] | 2-[(2S,4R)-4-fluoro-2-{[(S)-[3- fluoro-4-(1- methylcyclopropyl)phenyl](phenyl) methyl]carbamoyl} pyrrolidin-1-yl]- 2-oxoethyl azetidine-1-carboxylate |
| GZ-561 | 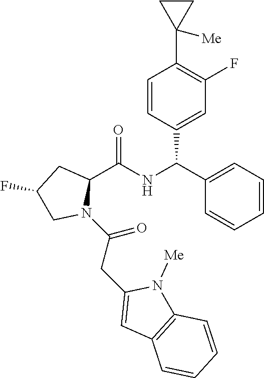 [SMILES: Cn1c(CC(=O)N2C[C@H](F)C[C@H]2C(=O)N[C@@H](c2ccccc2)c2ccc(C3(C)CC3)c(F)c2)cc2ccccc21] | (2S,4R)-4-fluoro-N-[(S)-[3-fluoro- 4-(1- methylcyclopropyl)phenyl] (phenyl) methyl]-1-[2-(1-methyl-1H-indol- 2-yl)acetyl]pyrrolidine-2- carboxamide |
| GZ-562 | 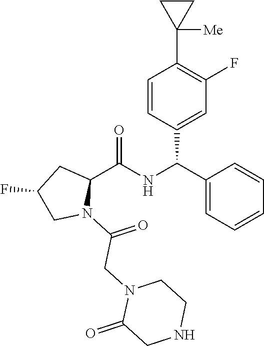 [SMILES: CC1(c2ccc([C@@H](NC(=O)[C@@H]3C[C@@H](F)CN3C(=O)CN3CCNCC3=O)c3ccccc3)cc2F)CC1] | (2S,4R)-4-fluoro-N-[(S)-[3-fluoro- 4-(1- methylcyclopropyl)phenyl](phenyl) methyl]-1-[2-(2-oxopiperazin-1- yl)acetyl]pyrrolidine-2- carboxamide |
| GZ-563 | 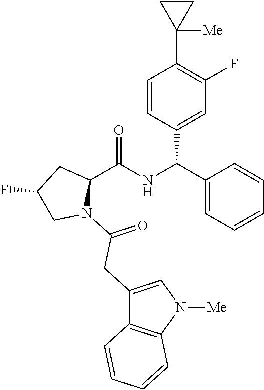 [SMILES: Cn1cc(CC(=O)N2C[C@H](F)C[C@H]2C(=O)N[C@@H](c2ccccc2)c2ccc(C3(C)CC3)c(F)c2)c2ccccc21] | (2S,4R)-4-fluoro-N-[(S)-[3-fluoro- 4-(1- methylcyclopropyl)phenyl] (phenyl) methyl]-1-[2-(1-methyl-1H-indol- 3-yl)acetyl]pyrrolidine-2- carboxamide |
| GZ-564 | 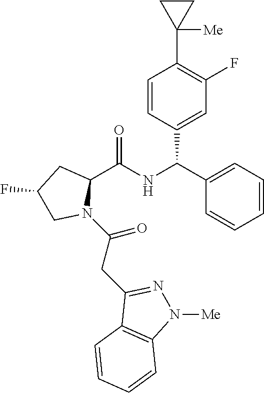 [SMILES: Cn1nc(CC(=O)N2C[C@H](F)C[C@H]2C(=O)N[C@@H](c2ccccc2)c2ccc(C3(C)CC3)c(F)c2)c2ccccc21] | (2S,4R)-4-fluoro-N-[(S)-[3-fluoro- 4-(1- methylcyclopropyl)phenyl] (phenyl) methyl]-1-[2-(1-methyl-1H- indazol-3-yl)acetyl]pyrrolidine-2- carboxamide |
| GZ-565 | 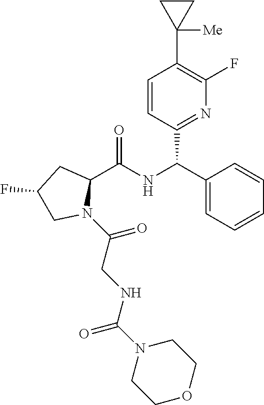 [SMILES: CC1(c2ccc([C@@H](NC(=O)[C@@H]3C[C@@H](F)CN3C(=O)CNC(=O)N3CCOCC3)c3ccccc3)nc2F)CC1] | N-{2-[(2S,4R)-4-fluoro-2-{[(S)-[3- fluoro-4-(1- methylcyclopropyl)phenyl] (phenyl) methyl] carbamoyl} pyrrolidin-1-yl]- 2-oxoethyl } morpholine-4- carboxamide |
| GZ-566 | 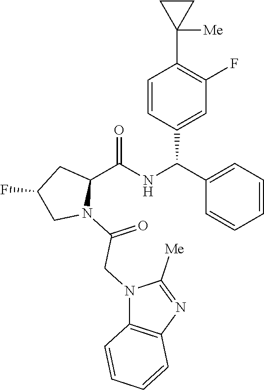 [SMILES: Cc1nc2ccccc2n1CC(=O)N1C[C@H](F)C[C@H]1C(=O)N[C@@H](c1ccccc1)c1ccc(C2(C)CC2)c(F)c1] | (2S,4R)-4-fluoro-N-[(S)-[3-fluoro- 4-(1- methylcyclopropyl)phenyl](phenyl) methyl]-1-[2-(2-methyl-1H-1,3- benzodiazol-1- yl)acetyl]pyrrolidine-2- carboxamide |
| GZ-567 | 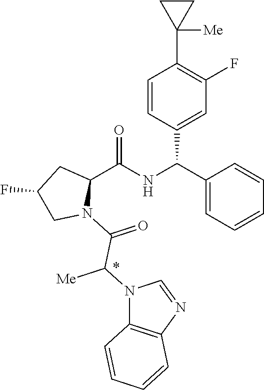 [SMILES: CC(C(=O)N1C[C@H](F)C[C@H]1C(=O)N[C@@H](c1ccccc1)c1ccc(C2(C)CC2)c(F)c1)n1cnc2ccccc21] | (2S,4R)-1-[(2R) or (2S)-2-(1H-1,3- benzodiazol-1-yl)propanoyl]-4- fluoro-N-[(S)-[3-fluoro-4-(1- methylcyclopropyl)phenyl](phenyl) methyl]pyrrolidine-2-carboxamide |
| GZ-568 | 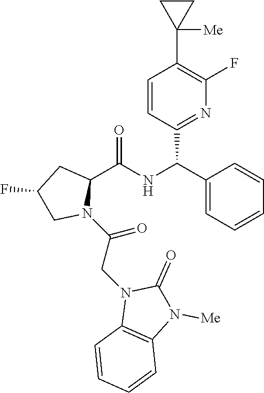 [SMILES: Cn1c(=O)n(CC(=O)N2C[C@H](F)C[C@H]2C(=O)N[C@@H](c2ccccc2)c2ccc(C3(C)CC3)c(F)n2)c2ccccc21] | (2S,4R)-4-fluoro-N-[(S)-[3-fluoro- 4-(1- methylcyclopropyl)phenyl] (phenyl) methyl]-1-[2-(3-methyl-2-oxo-2,3- dihydro-1H-1,3-benzodiazol-1- yl)acetyl]pyrrolidine-2- carboxamide |
| GZ-569 | 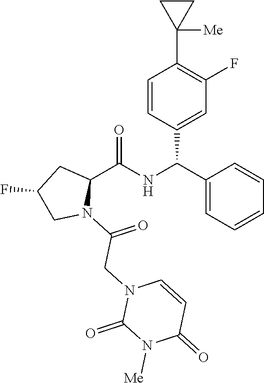 [SMILES: Cn1c(=O)ccn(CC(=O)N2C[C@H](F)C[C@H]2C(=O)N[C@@H](c2ccccc2)c2ccc(C3(C)CC3)c(F)c2)c1=O] | (2S,4R)-4-fluoro-N-[(S)-[3-fluoro- 4-(1- methylcyclopropyl)phenyl] (phenyl) methyl]-1-[2-(3-methyl-2,4-dioxo- 1,2,3,4-tetrahydropyrimidin-1- yl)acetyl]pyrrolidine-2- carboxamide |
| GZ-570 | 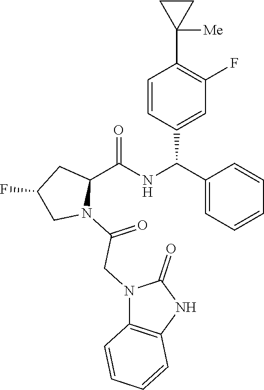 [SMILES: CC1(c2ccc([C@@H](NC(=O)[C@@H]3C[C@@H](F)CN3C(=O)Cn3c(=O)[nH]c4ccccc43)c3ccccc3)cc2F)CC1] | (2S,4R)-4-fluoro-N-[(S)-[3-fluoro- 4-(1- methylcyclopropyl)phenyl] (phenyl) methyl]-1-[2-(2-oxo-2,3-dihydro- 1H-1,3-benzodiazol-1- yl)acetyl]pyrrolidine-2- carboxamide |
| GZ-571 | 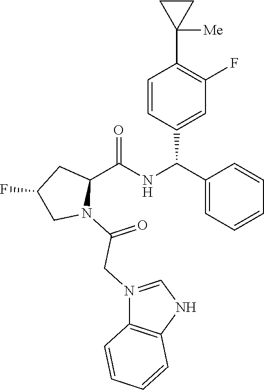 | (2S,4R)-4-fluoro-N-[(S)-[3-fluoro- 4-(1- methylcyclopropyl)phenyl](phenyl) methyl]-1-[2-(1H-indazol-3- yl)acetyl]pyrrolidine-2- carboxamide |
| GZ-572 | 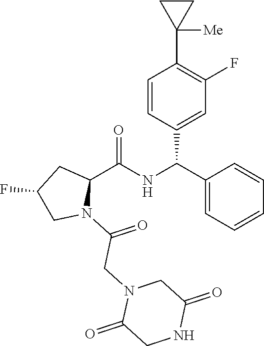 [SMILES: CC1(c2ccc([C@@H](NC(=O)[C@@H]3C[C@@H](F)CN3C(=O)CN3CC(=O)NCC3=O)c3ccccc3)cc2F)CC1] | (2S,4R)-1-[2-(2,5-dioxopiperazin- 1-yl)acetyl]-4-fluoro-N-[(S)-[3- fluoro-4-(1- methylcyclopropyl)phenyl](phenyl) methyl]pyrrolidine-2-carboxamide |
| GZ-573 | 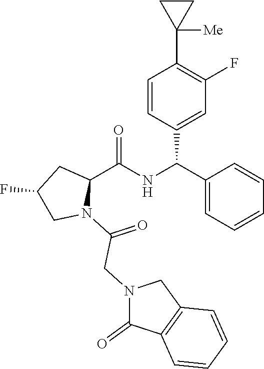 [SMILES: CC1(c2ccc([C@@H](NC(=O)[C@@H]3C[C@@H](F)CN3C(=O)CN3Cc4ccccc4C3=O)c3ccccc3)cc2F)CC1] | (2S,4R)-4-fluoro-N-[(S)-[3-fluoro- 4-(1- methylcyclopropyl)phenyl](phenyl) methyl]-1-[2-(1-oxo-2,3-dihydro- 1H-isoindol-2- yl)acetyl]pyrrolidine-2- carboxamide |
| GZ-574 | 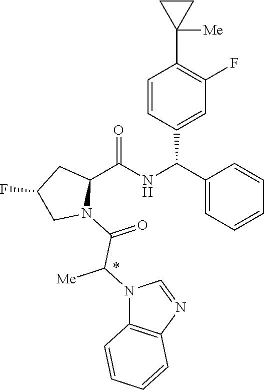 [SMILES: CC(C(=O)N1C[C@H](F)C[C@H]1C(=O)N[C@@H](c1ccccc1)c1ccc(C2(C)CC2)c(F)c1)n1cnc2ccccc21] | (2S,4R)-1-[(2S) or 2R)-2-(1H-1,3- benzodiazol-1-yl)propanoyl]-4- fluoro-N-[(S)-[3-fluoro-4-(1- methylcyclopropyl)phenyl](phenyl) methyl]pyrrolidine-2-carboxamide |
| GZ-575 | 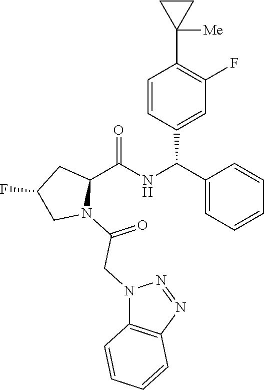 [SMILES: CC1(c2ccc([C@@H](NC(=O)[C@@H]3C[C@@H](F)CN3C(=O)Cn3nnc4ccccc43)c3ccccc3)cc2F)CC1] | (2S,4R)-1-[2-(1H-1,2,3- benzotriazol-1-yl)acetyl]-4-fluoro- N-[(S)-[3-fluoro-4-(1- methylcyclopropyl)phenyl](phenyl) methyl]pyrrolidine-2-carboxamide |
| GZ-576 | 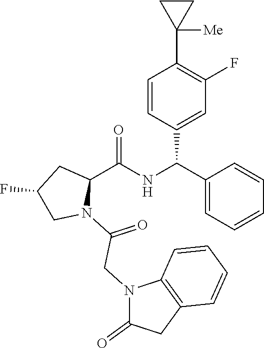 [SMILES: CC1(c2ccc([C@@H](NC(=O)[C@@H]3C[C@@H](F)CN3C(=O)CN3C(=O)Cc4ccccc43)c3ccccc3)cc2F)CC1] | (2S,4R)-4-fluoro-N-[(S)-[3-fluoro- 4-(1- methylcyclopropyl)phenyl](phenyl) methyl]-1-[2-(2-oxo-2,3-dihydro- 1H-indol-1-yl)acetyl]pyrrolidine-2- carboxamide |
| GZ-577 | 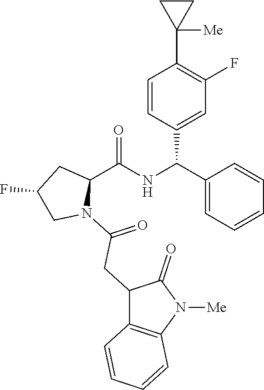 [SMILES: CN1C(=O)C(CC(=O)N2C[C@H](F)C[C@H]2C(=O)N[C@@H](c2ccccc2)c2ccc(C3(C)CC3)c(F)c2)c2ccccc21] | (2S,4R)-4-fluoro-N-[(S)-[3-fluoro- 4-(1- methylcyclopropyl)phenyl] (phenyl) methyl]-1-[2-(1-methyl-2-oxo-2,3- dihydro-1H-indol-3- yl)acetyl]pyrrolidine-2- carboxamide |
| GZ-578 | 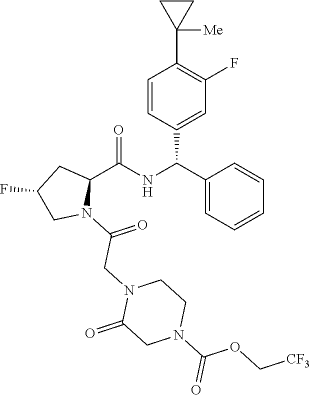 [SMILES: CC1(c2ccc([C@@H](NC(=O)[C@@H]3C[C@@H](F)CN3C(=O)CN3CCN(C(=O)OCC(F)(F)F)CC3=O)c3ccccc3)cc2F)CC1] | 2,2,2-trifluoroethyl 4-{2-[(2S,4R)- 4-fluoro-2-{[(S)-[3-fluoro-4-(1- methylcyclopropyl)phenyl] (phenyl) methyl]carbamoyl} pyrrolidin-1-yl]- 2-oxoethyl}-3-oxopiperazine-1- carboxylate |
| GZ-579 | 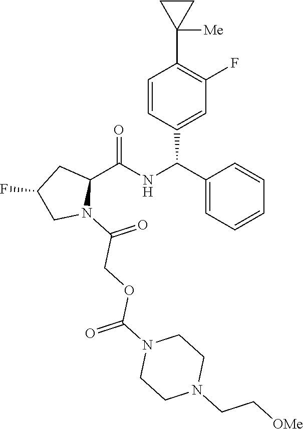 [SMILES: COCCN1CCN(C(=O)OCC(=O)N2C[C@H](F)C[C@H]2C(=O)N[C@@H](c2ccccc2)c2ccc(C3(C)CC3)c(F)c2)CC1] | 2-[(2S,4R)-4-fluoro-2-{[(S)-[3- fluoro-4-(1- methylcyclopropyl)phenyl] (phenyl) methyl]carbamoyl} pyrrolidin-1-yl]- 2-oxoethyl 4-(2- methoxyethyl)piperazine-1- carboxylate |
| GZ-580 | 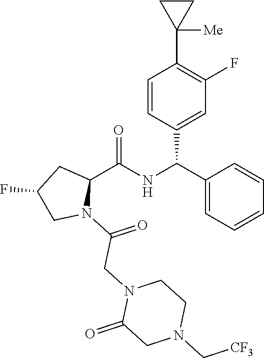 [SMILES: CC1(c2ccc([C@@H](NC(=O)[C@@H]3C[C@@H](F)CN3C(=O)CN3CCN(CC(F)(F)F)CC3=O)c3ccccc3)cc2F)CC1] | (2S,4R)-4-fluoro-N-[(S)-[3-fluoro- 4-(1- methylcyclopropyl)phenyl] (phenyl) methyl]-1-{2-[2-oxo-4-(2,2,2- trifluoroethyl)piperazin-1- yl]acetyl} pyrrolidine-2- carboxamide |
| GZ-581 | 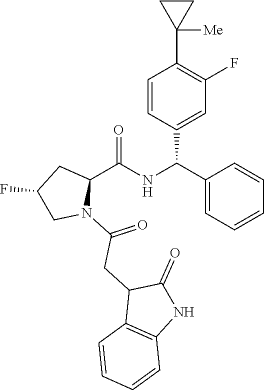 [SMILES: CC1(c2ccc([C@@H](NC(=O)[C@@H]3C[C@@H](F)CN3C(=O)CC3C(=O)Nc4ccccc43)c3ccccc3)cc2F)CC1] | (2S,4R)-4-fluoro-N-[(S)-[3-fluoro- 4-(1- methylcyclopropyl)phenyl](phenyl) methyl]-1-[2-(2-oxo-2,3-dihydro- 1H-indol-3-yl)acetyl]pyrrolidine-2- carboxamide |
| GZ-582 | 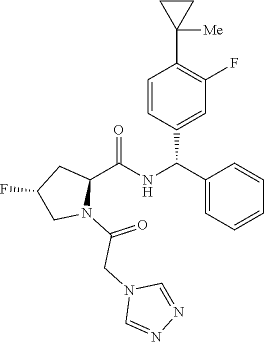 [SMILES: CC1(c2ccc([C@@H](NC(=O)[C@@H]3C[C@@H](F)CN3C(=O)Cn3cnnc3)c3ccccc3)cc2F)CC1] | (2S,4R)-4-fluoro-N-[(S)-[3-fluoro- 4-(1- methylcyclopropyl)phenyl](phenyl) methyl]-1-[2-(4H-1,2,4-triazol-4- yl)acetyl]pyrrolidine-2- carboxamide |
| GZ-583 | 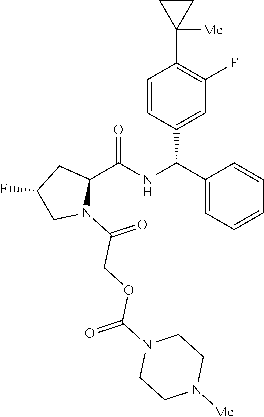 [SMILES: CN1CCN(C(=O)OCC(=O)N2C[C@H](F)C[C@H]2C(=O)N[C@@H](c2ccccc2)c2ccc(C3(C)CC3)c(F)c2)CC1] | 2-[(2S,4R)-4-fluoro-2-{[(S)-[3- fluoro-4-(1- methylcyclopropyl)phenyl] (phenyl) methyl] carbamoyl} pyrrolidin-1-yl]- 2-oxoethyl 4-methylpiperazine-1- carboxylate |
| GZ-584 | 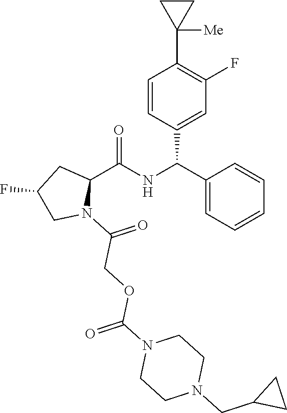 [SMILES: CC1(c2ccc([C@@H](NC(=O)[C@@H]3C[C@@H](F)CN3C(=O)COC(=O)N3CCN(CC4CC4)CC3)c3ccccc3)cc2F)CC1] | 2-[(2S,4R)-4-fluoro-2-{[(S)-[3- fluoro-4-(1- methylcyclopropyl)phenyl] (phenyl) methyl] carbamoyl } pyrrolidin-1-yl]- 2-oxoethyl 4- (cyclopropylmethyl)piperazine-1- carboxylate |
| GZ-585 | 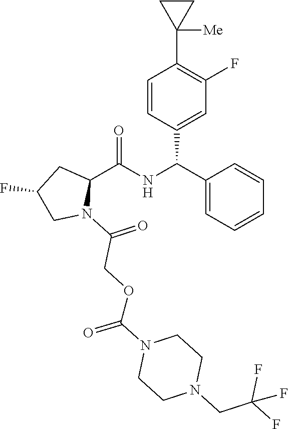 [SMILES: CC1(c2ccc([C@@H](NC(=O)[C@@H]3C[C@@H](F)CN3C(=O)COC(=O)N3CCN(CC(F)(F)F)CC3)c3ccccc3)cc2F)CC1] | 2-[(2S,4R)-4-fluoro-2-{[(S)-[3- fluoro-4-(1- methylcyclopropyl)phenyl] (phenyl) methyl]carbamoyl } pyrrolidin-1-yl]- 2-oxoethyl 4-(2,2,2- trifluoroethyl)piperazine-1- carboxylate |
| GZ-586 | 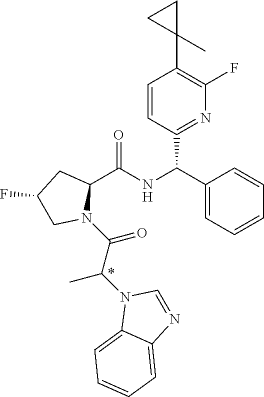 [SMILES: CC(C(=O)N1C[C@H](F)C[C@H]1C(=O)N[C@@H](c1ccccc1)c1ccc(C2(C)CC2)c(F)n1)n1cnc2ccccc21] | (2S,4R)-1-((R) or (S)-2-(1H- benzo[d]imidazol-1-yl)propanoyl)- 4-fluoro-N-((S)-(6-fluoro-5-(1- methylcyclopropyl)pyridin-2- yl)(phenyl)methyl)pyrrolidine-2- carboxamide |
| GZ-587 | 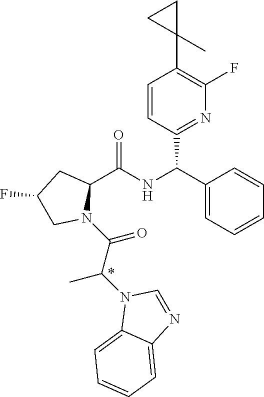 [SMILES: CC(C(=O)N1C[C@H](F)C[C@H]1C(=O)N[C@@H](c1ccccc1)c1ccc(C2(C)CC2)c(F)n1)n1cnc2ccccc21] | (2S,4R)-1-((S) or (R)-2-(1H- benzo[d]imidazol-1-yl)propanoyl)- 4-fluoro-N-((S)-(6-fluoro-5-(1- methylcyclopropyl)pyridin-2- yl)(phenyl)methyl)pyrrolidine-2- carboxamide |
| GZ-588 | 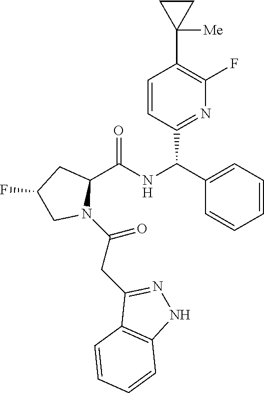 [SMILES: CC1(c2ccc([C@@H](NC(=O)[C@@H]3C[C@@H](F)CN3C(=O)Cc3n[nH]c4ccccc34)c3ccccc3)nc2F)CC1] | (2S,4R)-4-fluoro-N-[(S)-[6-fluoro- 5-(1-methylcyclopropyl)pyridin-2- yl](phenyl)methyl]-1-[2-(1H- indazol-3-yl)acetyl]pyrrolidine-2- carboxamide |
| GZ-589 | 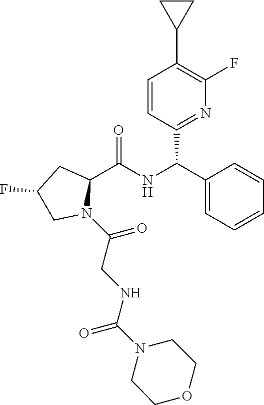 [SMILES: O=C(N[C@@H](c1ccccc1)c1ccc(C2CC2)c(F)n1)[C@@H]1C[C@@H](F)CN1C(=O)CNC(=O)N1CCOCC1] | N-{2-[(2S,4R)-4-fluoro-2-{[(S)-[6- fluoro-5-(1- methylcyclopropyl)pyridin-2- yl](phenyl)methyl]carbamoyl} pyrr olidin-1-yl]-2- oxoethyl } morpholine-4- carboxamide |
| GZ-590 | 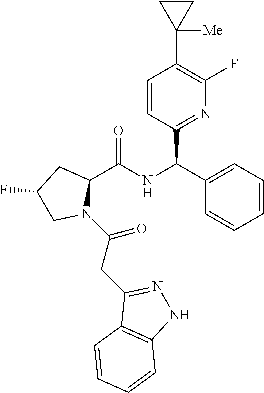 [SMILES: CC1(c2ccc([C@H](NC(=O)[C@@H]3C[C@@H](F)CN3C(=O)Cc3n[nH]c4ccccc34)c3ccccc3)nc2F)CC1] | (2S,4R)-4-fluoro-N-[(R)-[6-fluoro- 5-(1-methylcyclopropyl)pyridin-2- yl](phenyl)methyl]-1-[2-(1H- indazol-3-yl)acetyl]pyrrolidine-2- carboxamide |
| GZ-591 | 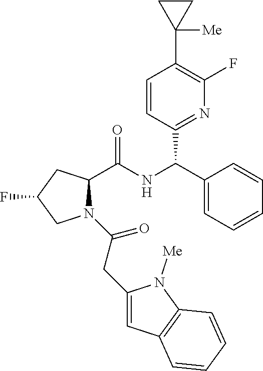 [SMILES: Cn1c(CC(=O)N2C[C@H](F)C[C@H]2C(=O)N[C@@H](c2ccccc2)c2ccc(C3(C)CC3)c(F)n2)cc2ccccc21] | (2S,4R)-4-fluoro-N-[(S)-[6-fluoro- 5-(1-methylcyclopropyl)pyridin-2- yl](phenyl)methyl]-1-[2-(1-methyl- 1H-indol-2-yl)acetyl]pyrrolidine-2- carboxamide |
| GZ-592 | 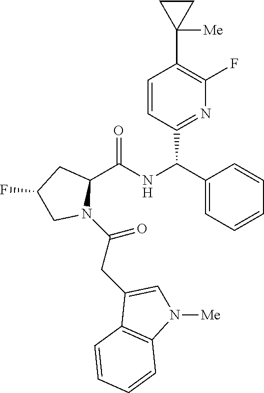 [SMILES: Cn1cc(CC(=O)N2C[C@H](F)C[C@H]2C(=O)N[C@@H](c2ccccc2)c2ccc(C3(C)CC3)c(F)n2)c2ccccc21] | (2S,4R)-4-fluoro-N-[(S)-[6-fluoro- 5-(1-methylcyclopropyl)pyridin-2- yl](phenyl)methyl]-1-[2-(1-methyl- 1H-indol-3-yl)acetyl]pyrrolidine-2- carboxamide |
| GZ-593 | 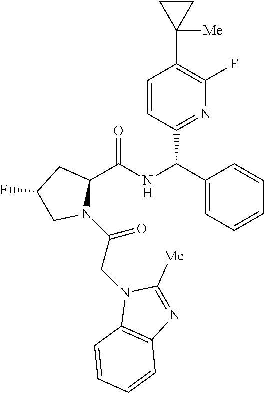 [SMILES: Cc1nc2ccccc2n1CC(=O)N1C[C@H](F)C[C@H]1C(=O)N[C@@H](c1ccccc1)c1ccc(C2(C)CC2)c(F)n1] | (2S,4R)-4-fluoro-N-[(S)-[6-fluoro- 5-(1-methylcyclopropyl)pyridin-2- yl](phenyl)methyl]-1-[2-(2-methyl- 1H-1,3-benzodiazol-1- yl)acetyl]pyrrolidine-2- carboxamide |
| GZ-594 | 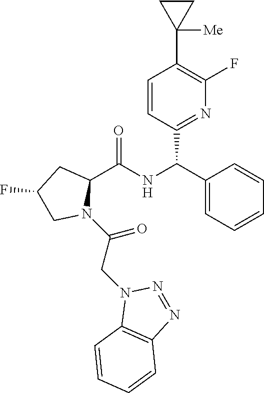 [SMILES: CC1(c2ccc([C@@H](NC(=O)[C@@H]3C[C@@H](F)CN3C(=O)Cn3nnc4ccccc43)c3ccccc3)nc2F)CC1] | (2S,4R)-1-[2-(1H-1,2,3- benzotriazol-1-yl)acetyl]-4-fluoro- N-[(S)-[6-fluoro-5-(1- methylcyclopropyl)pyridin-2- yl](phenyl)methyl]pyrrolidine-2- carboxamide |
| GZ-595 | 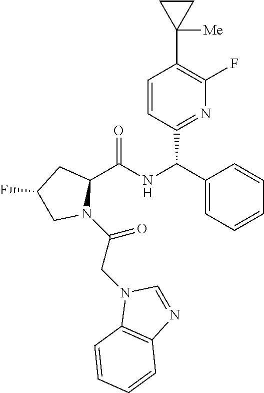 [SMILES: CC1(c2ccc([C@@H](NC(=O)[C@@H]3C[C@@H](F)CN3C(=O)Cn3cnc4ccccc43)c3ccccc3)nc2F)CC1] | (2S,4R)-1-[2-(1H-1,3-benzodiazol- 1-yl)acetyl]-4-fluoro-N-[(S)-[6- fluoro-5-(1- methylcyclopropyl)pyridin-2- yl](phenyl)methyl]pyrrolidine-2- carboxamide |
| GZ-596 | 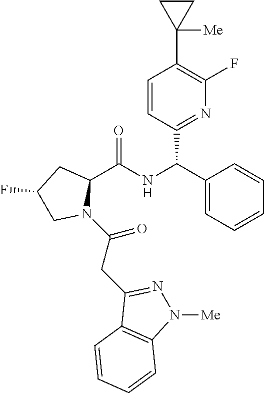 [SMILES: Cn1nc(CC(=O)N2C[C@H](F)C[C@H]2C(=O)N[C@@H](c2ccccc2)c2ccc(C3(C)CC3)c(F)n2)c2ccccc21] | (2S,4R)-4-fluoro-N-[(S)-[6-fluoro- 5-(1-methylcyclopropyl)pyridin-2- yl](phenyl)methyl]-1-[2-(1-methyl- 1H-indazol-3-yl)acetyl]pyrrolidine- 2-carboxamide |
| GZ-597 | 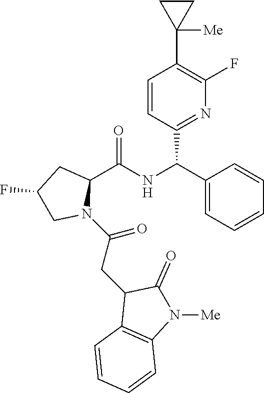 [SMILES: CN1C(=O)C(CC(=O)N2C[C@H](F)C[C@H]2C(=O)N[C@@H](c2ccccc2)c2ccc(C3(C)CC3)c(F)n2)c2ccccc21] | (2S,4R)-4-fluoro-N-[(S)-[6-fluoro- 5-(1-methylcyclopropyl)pyridin-2- yl](phenyl)methyl]-1-[2-(1-methyl- 2-oxo-2,3-dihydro-1H-indol-3- yl)acetyl]pyrrolidine-2- carboxamide |
| GZ-598 | 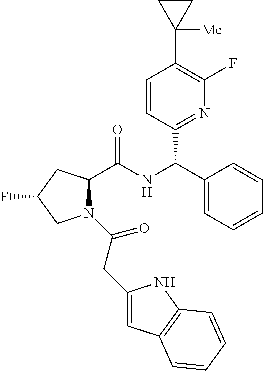 [SMILES: CC1(c2ccc([C@@H](NC(=O)[C@@H]3C[C@@H](F)CN3C(=O)Cc3cc4ccccc4[nH]3)c3ccccc3)nc2F)CC1] | (2S,4R)-4-fluoro-N-[(S)-[6-fluoro- 5-(1-methylcyclopropyl)pyridin-2- yl](phenyl)methyl]-1-[2-(1H-indol- 2-yl)acetyl]pyrrolidine-2- carboxamide |
| GZ-599 | 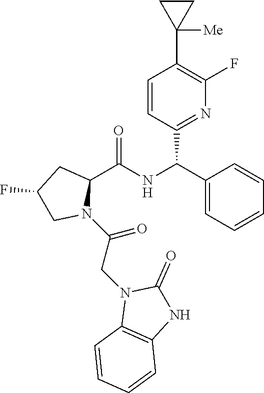 [SMILES: CC1(c2ccc([C@@H](NC(=O)[C@@H]3C[C@@H](F)CN3C(=O)Cn3c(=O)[nH]c4ccccc43)c3ccccc3)nc2F)CC1] | (2S,4R)-4-fluoro-N-[(S)-[6-fluoro- 5-(1-methylcyclopropyl)pyridin-2- yl](phenyl)methyl]-1-[2-(2-oxo- 2,3-dihydro-1H-1,3-benzodiazol-1- yl)acetyl]pyrrolidine-2- carboxamide |
| GZ-600 | 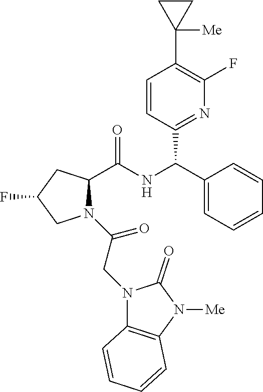 [SMILES: Cn1c(=O)n(CC(=O)N2C[C@H](F)C[C@H]2C(=O)N[C@@H](c2ccccc2)c2ccc(C3(C)CC3)c(F)n2)c2ccccc21] | (2S,4R)-4-fluoro-N-[(S)-[6-fluoro- 5-(1-methylcyclopropyl)pyridin-2- yl](phenyl)methyl]-1-[2-(3-methyl- 2-oxo-2,3-dihydro-1H-1,3- benzodiazol-1- yl)acetyl]pyrrolidine-2- carboxamide |
| GZ-601 | 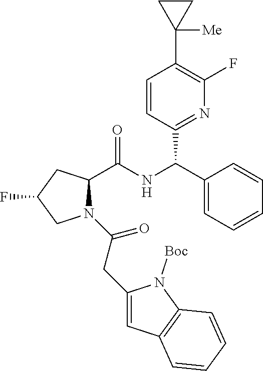 [SMILES: CC(C)(C)OC(=O)n1c(CC(=O)N2C[C@H](F)C[C@H]2C(=O)N[C@@H](c2ccccc2)c2ccc(C3(C)CC3)c(F)n2)cc2ccccc21] | tert-butyl 2-{2-[(2S,4R)-4-fluoro-2- {[(S)-[6-fluoro-5-(1- methylcyclopropyl)pyridin-2- yl](phenyl)methyl]carbamoyl}pyrr olidin-1-yl]-2-oxoethyl}-1H- indole-1-carboxylate |
| GZ-602 | 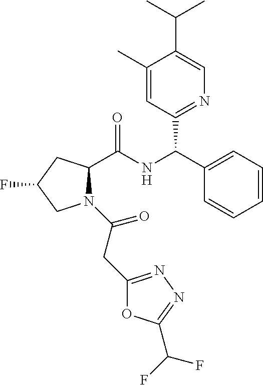 [SMILES: Cc1cc([C@@H](NC(=O)[C@@H]2C[C@@H](F)CN2C(=O)Cc2nnc(C(F)F)o2)c2ccccc2)ncc1C(C)C] | (2S,4R)-1-(2-(5-(difluoromethyl)- 1,3,4-oxadiazol-2-yl)acetyl)-4- fluoro-N-((S)-(5-isopropyl-4- methylpyridin-2- yl)(phenyl)methyl)pyrrolidine-2- carboxamide |
| GZ-603 | 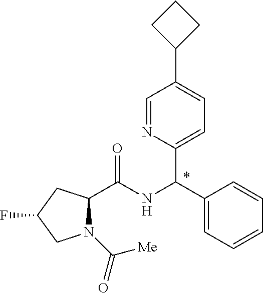 [SMILES: CC(=O)N1C[C@H](F)C[C@H]1C(=O)NC(c1ccccc1)c1ccc(C2CCC2)cn1] | (2S,4R)-1-acetyl-N-[(S) or (R)-(5- cyclobutylpyridin-2- yl)(phenyl)methyl]-4- fluoropyrrolidine-2-carboxamide |
| GZ-604 | 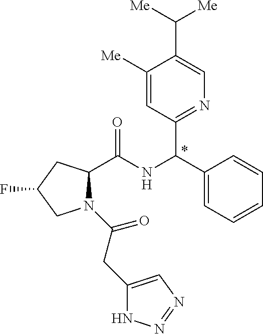 [SMILES: Cc1cc(C(NC(=O)[C@@H]2C[C@@H](F)CN2C(=O)Cc2cnn[nH]2)c2ccccc2)ncc1C(C)C] | (2S,4R)-4-fluoro-N-[(S) or (R)-[4- methyl-5-(propan-2-yl)pyridin-2- yl](phenyl)methyl]-1-[2-(1H-1,2,3- triazol-5-yl)acetyl]pyrrolidine-2- carboxamide |
| GZ-605 | 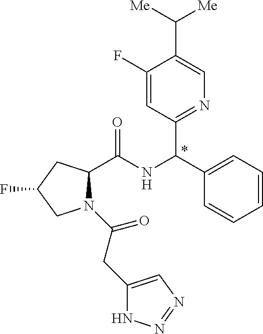 [SMILES: CC(C)c1cnc(C(NC(=O)[C@@H]2C[C@@H](F)CN2C(=O)Cc2cnn[nH]2)c2ccccc2)cc1F] | (2S,4R)-4-fluoro-N-[(S) or (R)-[4- fluoro-5-(propan-2-yl)pyridin-2- yl](phenyl)methyl]-1-[2-(1H-1,2,3- triazol-5-yl)acetyl]pyrrolidine-2- carboxamide |
| GZ-606 | 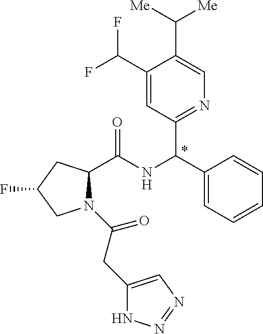 [SMILES: CC(C)c1cnc(C(NC(=O)[C@@H]2C[C@@H](F)CN2C(=O)Cc2cnn[nH]2)c2ccccc2)cc1C(F)F] | (2S,4R)-N-[(S) or (R)-[4- (difluoromethyl)-5-(propan-2- yl)pyridin-2-yl](phenyl)methyl]-4- fluoro-1-[2-(1H-1,2,3-triazol-5- yl)acetyl]pyrrolidine-2- carboxamide |
| GZ-607 | 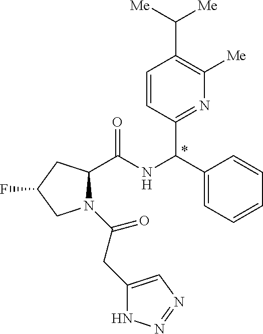 [SMILES: Cc1nc(C(NC(=O)[C@@H]2C[C@@H](F)CN2C(=O)Cc2cnn[nH]2)c2ccccc2)ccc1C(C)C] | (2S,4R)-4-fluoro-N-[(S) or (R)-[6- methyl-5-(propan-2-yl)pyridin-2- yl](phenyl)methyl]-1-[2-(1H-1,2,3- triazol-5-yl)acetyl]pyrrolidine-2- carboxamide |
| GZ-608 | 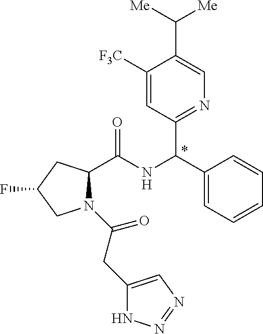 [SMILES: CC(C)c1cnc(C(NC(=O)[C@@H]2C[C@@H](F)CN2C(=O)Cc2cnn[nH]2)c2ccccc2)cc1C(F)(F)F] | (2S,4R)-4-fluoro-N-[(S) or (R)- phenyl[5-(propan-2-yl)-4- (trifluoromethyl)pyridin-2- yl]methyl]-1-[2-(1H-1,2,3-triazol- 5-yl)acetyl]pyrrolidine-2- carboxamide |
| GZ-609 | 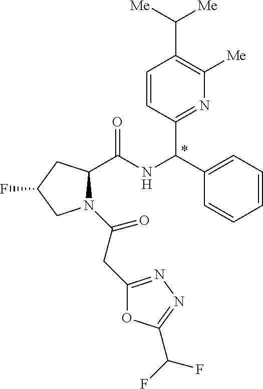 [SMILES: Cc1nc(C(NC(=O)[C@@H]2C[C@@H](F)CN2C(=O)Cc2nnc(C(F)F)o2)c2ccccc2)ccc1C(C)C] | (2S,4R)-1-{2-[5-(difluoromethyl)- 1,3,4-oxadiazol-2-yl]acetyl}-4- fluoro-N-[(S) or (R)-[6-methyl-5- (propan-2-yl)pyridin-2- yl](phenyl)methyl]pyrrolidine-2- carboxamide |
| GZ-610 | 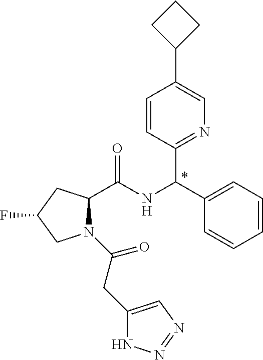 [SMILES: O=C(NC(c1ccccc1)c1ccc(C2CCC2)cn1)[C@@H]1C[C@@H](F)CN1C(=O)Cc1cnn[nH]1] | (2S,4R)-N-[(S) or (R)-(5- cyclobutylpyridin-2- yl)(phenyl)methyl]-4-fluoro-1-[2- (1H-1,2,3-triazol-5- yl)acetyl]pyrrolidine-2- carboxamide |
| GZ-611 | 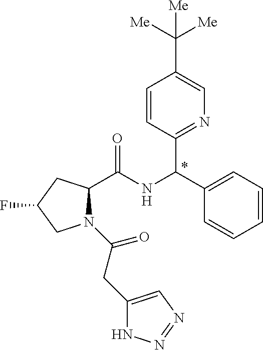 [SMILES: CC(C)(C)c1ccc(C(NC(=O)[C@@H]2C[C@@H](F)CN2C(=O)Cc2cnn[nH]2)c2ccccc2)nc1] | (2S,4R)-N-[(S) or (R)-(5-tert- butylpyridin-2-yl)(phenyl)methyl]- 4-fluoro-1-[2-(1H-1,2,3-triazol-5- yl)acetyl]pyrrolidine-2- carboxamide |
| GZ-612 | 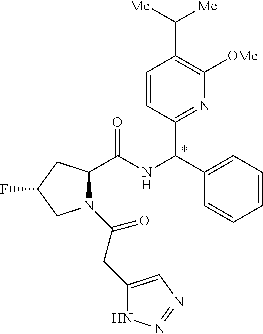 [SMILES: COc1nc(C(NC(=O)[C@@H]2C[C@@H](F)CN2C(=O)Cc2cnn[nH]2)c2ccccc2)ccc1C(C)C] | (2S,4R)-4-fluoro-N-[(S) or (R)-[6- methoxy-5-(propan-2-yl)pyridin-2- yl](phenyl)methyl]-1-[2-(1H-1,2,3- triazol-5-yl)acetyl]pyrrolidine-2- carboxamide |
| GZ-613 | 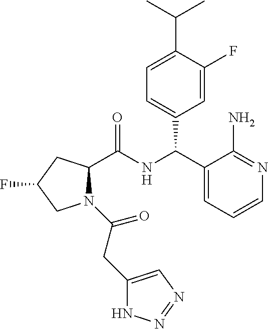 [SMILES: CC(C)c1ccc([C@@H](NC(=O)[C@@H]2C[C@@H](F)CN2C(=O)Cc2cnn[nH]2)c2cccnc2N)cc1F] | (2S,4R)-1-(2-(1H-1,2,3-triazol-5- yl)acetyl)-N-((R)-(2-aminopyridin- 3-yl)(3-fluoro-4- isopropylphenyl)methyl)-4- fluoropyrrolidine-2-carboxamide |
| GZ-614 | 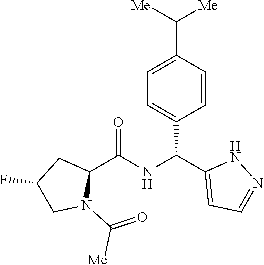 [SMILES: CC(=O)N1C[C@H](F)C[C@H]1C(=O)N[C@H](c1ccc(C(C)C)cc1)c1ccn[nH]1] | (2S,4R)-1-acetyl-4-fluoro-N-[(S)- [4-(propan-2-yl)phenyl] (1H- pyrazol-5-yl)methyl]pyrrolidine-2- carboxamide |
| GZ-615 | 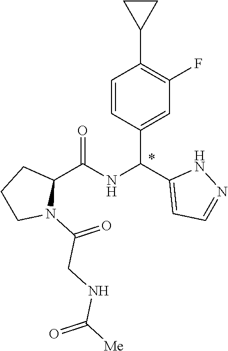 [SMILES: CC(=O)NCC(=O)N1CCC[C@H]1C(=O)NC(c1ccc(C2CC2)c(F)c1)c1ccn[nH]1] | (2S)-N-[(R) or (S)-(4-cyclopropyl- 3-fluorophenyl)(1H-pyrazol-5- yl)methyl]-1-(2- acetamidoacetyl)pyrrolidine-2- carboxamide |
| GZ-616 | 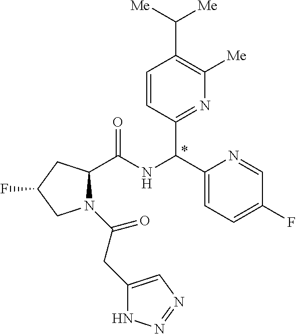 [SMILES: Cc1nc(C(NC(=O)[C@@H]2C[C@@H](F)CN2C(=O)Cc2cnn[nH]2)c2ccc(F)cn2)ccc1C(C)C] | (2S,4R)-4-fluoro-N-[(R) or (S)-[3- fluoro-4-(propan-2-yl)phenyl](5- fluoropyridin-2-yl)methyl]-1-[2- (1H-1,2,3-triazol-5- yl)acetyl]pyrrolidine-2- carboxamide |
| GZ-617 | 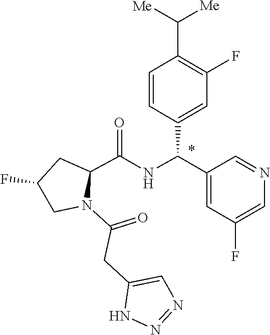 [SMILES: CC(C)c1ccc([C@@H](NC(=O)[C@@H]2C[C@@H](F)CN2C(=O)Cc2cnn[nH]2)c2cncc(F)c2)cc1F] | (2S,4R)-4-fluoro-N-[(R) or (S)-[3- fluoro-4-(propan-2-yl)phenyl](5- fluoropyridin-3-yl)methyl]-1-[2- (1H-1,2,3-triazol-5- yl)acetyl]pyrrolidine-2- carboxamide |
| GZ-618 | 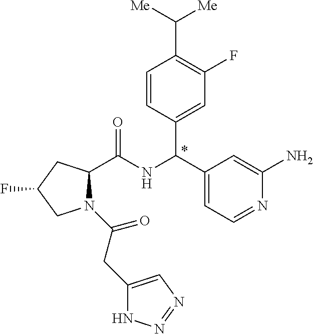 [SMILES: CC(C)c1ccc(C(NC(=O)[C@@H]2C[C@@H](F)CN2C(=O)Cc2cnn[nH]2)c2ccnc(N)c2)cc1F] | (2S,4R)-N-[(S) or (R)-(2- aminopyridin-4-yl)[3-fluoro-4- (propan-2-yl)phenyl]methyl]-4- fluoro-1-[2-(1H-1,2,3-triazol-5- yl)acetyl]pyrrolidine-2- carboxamide |
| GZ-619 | 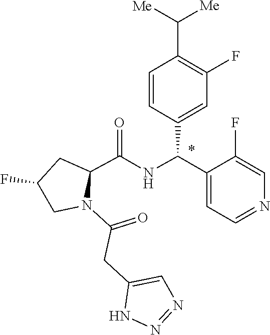 [SMILES: CC(C)c1ccc([C@@H](NC(=O)[C@@H]2C[C@@H](F)CN2C(=O)Cc2cnn[nH]2)c2ccncc2F)cc1F] | (2S,4R)-4-fluoro-N-[(R) or (S)-[3- fluoro-4-(propan-2-yl)phenyl](3- fluoropyridin-4-yl)methyl]-1-[2- (1H-1,2,3-triazol-5- yl)acetyl]pyrrolidine-2- carboxamide |
| GZ-620 | 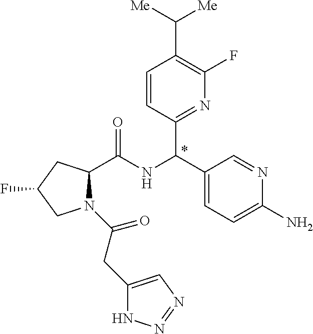 [SMILES: CC(C)c1ccc(C(NC(=O)[C@@H]2C[C@@H](F)CN2C(=O)Cc2cnn[nH]2)c2ccc(N)nc2)nc1F] | (2S,4R)-N-[(R) or (S)-(6- aminopyridin-3-yl)[3-fluoro-4- (propan-2-yl)phenyl]methyl]-4- fluoro-1-[2-(1H-1,2,3-triazol-5- yl)acetyl]pyrrolidine-2- carboxamide |
| GZ-621 | 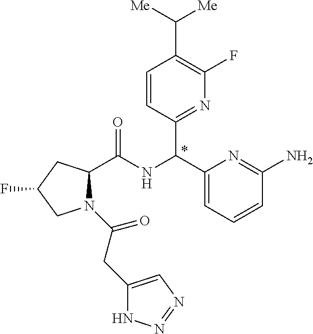 [SMILES: CC(C)c1ccc(C(NC(=O)[C@@H]2C[C@@H](F)CN2C(=O)Cc2cnn[nH]2)c2cccc(N)n2)nc1F] | (2S,4R)-N-[(R) or (S)-(6- aminopyridin-2-yl)[3-fluoro-4- (propan-2-yl)phenyl]methyl]-4- fluoro-1-[2-(1H-1,2,3-triazol-5- yl)acetyl]pyrrolidine-2- carboxamide |
| GZ-622 | 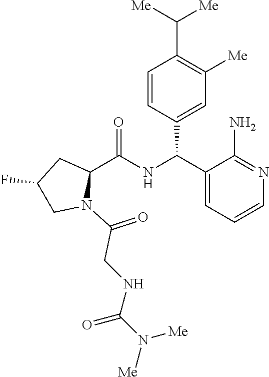 [SMILES: Cc1cc([C@@H](NC(=O)[C@@H]2C[C@@H](F)CN2C(=O)CNC(=O)N(C)C)c2cccnc2N)ccc1C(C)C] | (2S,4R)-N-[(R) or (S)-(2- aminopyridin-3-yl)[3-methyl-4- (propan-2-yl)phenyl]methyl]-1-{2- [(dimethylcarbamoyl)amino]acetyl}- 4-fluoropyrrolidine-2-carboxamide |
| GZ-623 | 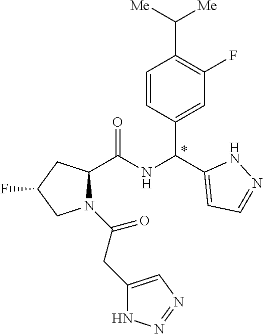 [SMILES: CC(C)c1ccc(C(NC(=O)[C@@H]2C[C@@H](F)CN2C(=O)Cc2cnn[nH]2)c2ccn[nH]2)cc1F] | (2S,4R)-4-fluoro-N-[(R) or (S)-[3- fluoro-4-(propan-2-yl)phenyl](1H- pyrazol-5-yl)methyl]-1-[2-(1H- 1,2,3-triazol-5- yl)acetyl]pyrrolidine-2- carboxamide |
| GZ-624 | 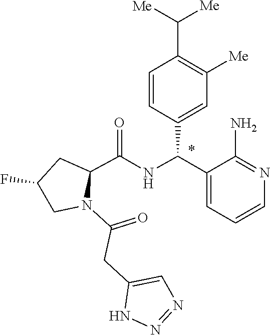 [SMILES: Cc1cc([C@@H](NC(=O)[C@@H]2C[C@@H](F)CN2C(=O)Cc2cnn[nH]2)c2cccnc2N)ccc1C(C)C] | (2S,4R)-N-[(R) or (S)-(2- aminopyridin-3-yl)[3-methyl-4- (propan-2-yl)phenyl]methyl]-4- fluoro-1-[2-(1H-1,2,3-triazol-5- yl)acetyl]pyrrolidine-2- carboxamide |
| GZ-625 | 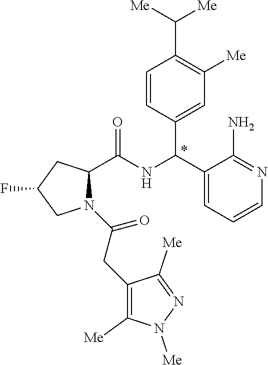 [SMILES: Cc1cc(C(NC(=O)[C@@H]2C[C@@H](F)CN2C(=O)Cc2c(C)nn(C)c2C)c2cccnc2N)ccc1C(C)C] | (2S,4R)-N-[(R) or (S)-(2- aminopyridin-3-yl)[3-methyl-4- (propan-2-yl)phenyl]methyl]-4- fluoro-1-[2-(1,3,5-trimethyl-1H- pyrazol-4-yl)acetyl]pyrrolidine-2- carboxamide |
| GZ-626 | 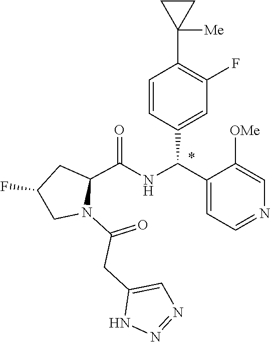 [SMILES: COc1cnccc1[C@H](NC(=O)[C@@H]1C[C@@H](F)CN1C(=O)Cc1cnn[nH]1)c1ccc(C2(C)CC2)c(F)c1] | (2S,4R)-4-fluoro-N-[(R) or (S)-[3- fluoro-4-(1- methylcyclopropyl)phenyl](2- methoxypyridin-3-yl)methyl]-1-[2- (1H-1,2,3-triazol-5- yl)acetyl]pyrrolidine-2- carboxamide |
| GZ-627 | 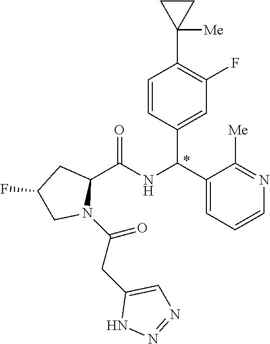 [SMILES: Cc1ncccc1C(NC(=O)[C@@H]1C[C@@H](F)CN1C(=O)Cc1cnn[nH]1)c1ccc(C2(C)CC2)c(F)c1] | (2S,4R)-4-fluoro-N-[(R) or (S)-[3- fluoro-4-(1- methylcyclopropyl)phenyl] (2- methylpyridin-3-yl)methyl]-1-[2- (1H-1,2,3-triazol-5- yl)acetyl]pyrrolidine-2- carboxamide |
| GZ-628 | 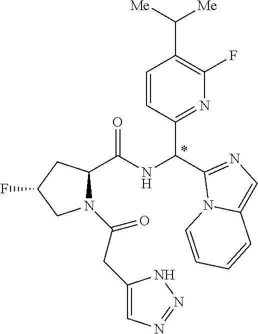 [SMILES: CC(C)c1ccc(C(NC(=O)[C@@H]2C[C@@H](F)CN2C(=O)Cc2cnn[nH]2)c2ncc3ccccn23)nc1F] | (2S,4R)-4-fluoro-N-[(R) or (S)-[6- fluoro-5-(propan-2-yl)pyridin-2- yl]({imidazo[1,5-a]pyridin-3- yl})methyl]-1-[2-(1H-1,2,3-triazol- 5-yl)acetyl]pyrrolidine-2- carboxamide |
| GZ-629 | 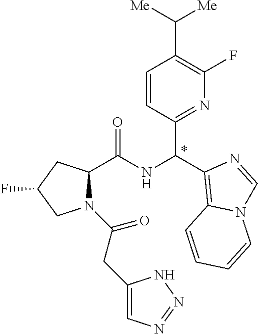 [SMILES: CC(C)c1ccc(C(NC(=O)[C@@H]2C[C@@H](F)CN2C(=O)Cc2cnn[nH]2)c2ncn3ccccc23)nc1F] | (2S,4R)-4-fluoro-N-[(S) or (R)-[6- fluoro-5-(propan-2-yl)pyridin-2- yl]({imidazo[1,5-a]pyridin-1- yl})methyl]-1-[2-(1H-1,2,3-triazol- 5-yl)acetyl]pyrrolidine-2- carboxamide |
| GZ-630 | 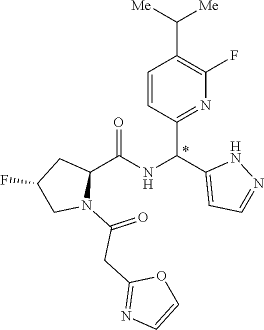 [SMILES: CC(C)c1ccc(C(NC(=O)[C@@H]2C[C@@H](F)CN2C(=O)Cc2ncco2)c2ccn[nH]2)nc1F] | (2S,4R)-4-fluoro-N-[(S) or (R)-[6- fluoro-5-(propan-2-yl)pyridin-2- yl](1H-pyrazol-5-yl)methyl]-1-[2- (1,3-oxazol-2-yl)acetyl]pyrrolidine- 2-carboxamide |
| GZ-631 | 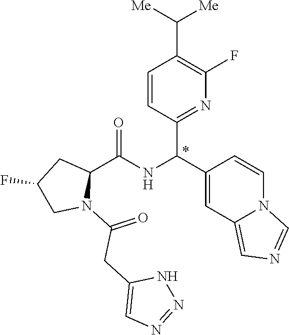 [SMILES: CC(C)c1ccc(C(NC(=O)[C@@H]2C[C@@H](F)CN2C(=O)Cc2cnn[nH]2)c2ccn3cncc3c2)nc1F] | (2S,4R)-4-fluoro-N-[(S) or (R)-[6- fluoro-5-(propan-2-yl)pyridin-2- yl]({imidazo[1,5-a]pyridin-7- yl})methyl]-1-[2-(1H-1,2,3-triazol- 5-yl)acetyl]pyrrolidine-2- carboxamide |
| GZ-632 | 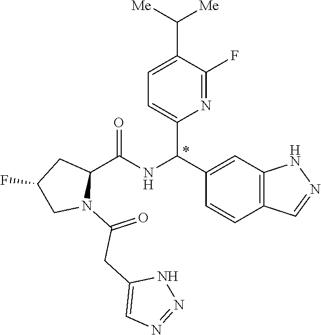 [SMILES: CC(C)c1ccc(C(NC(=O)[C@@H]2C[C@@H](F)CN2C(=O)Cc2cnn[nH]2)c2ccc3cn[nH]c3c2)nc1F] | (2S,4R)-4-fluoro-N-[(S) or (R)-[6- fluoro-5-(propan-2-yl)pyridin-2- yl](1H-indazol-6-yl)methyl]-1-[2- (1H-1,2,3-triazol-5- yl)acetyl]pyrrolidine-2- carboxamide |
| GZ-633 | 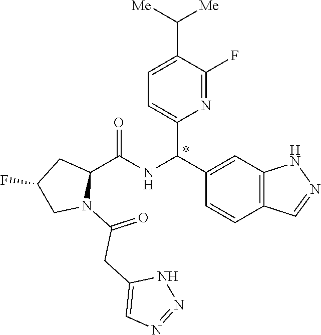 [SMILES: CC(C)c1ccc(C(NC(=O)[C@@H]2C[C@@H](F)CN2C(=O)Cc2cnn[nH]2)c2ccc3cn[nH]c3c2)nc1F] | (2S,4R)-4-fluoro-N-[(R) or (S)-[6- fluoro-5-(propan-2-yl)pyridin-2- yl](1H-indazol-6-yl)methyl]-1-[2- (1H-1,2,3-triazol-5- yl)acetyl]pyrrolidine-2- carboxamide |
| GZ-634 | 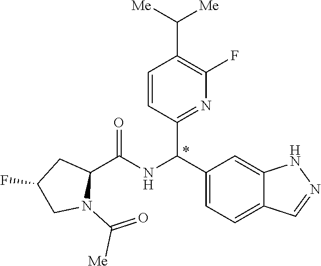 [SMILES: CC(=O)N1C[C@H](F)C[C@H]1C(=O)NC(c1ccc2cn[nH]c2c1)c1ccc(C(C)C)c(F)n1] | (2S,4R)-1-acetyl-4-fluoro-N-[(S) or (R)-[6-fluoro-5-(propan-2- yl)pyridin-2-yl](1H-indazol-6- yl)methyl]pyrrolidine-2- carboxamide |
| GZ-635 | 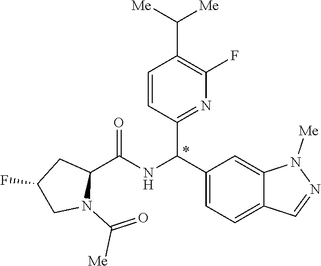 [SMILES: CC(=O)N1C[C@H](F)C[C@H]1C(=O)NC(c1ccc2cnn(C)c2c1)c1ccc(C(C)C)c(F)n1] | (2S,4R)-1-acetyl-4-fluoro-N-[(S) or (R)-[6-fluoro-5-(propan-2- yl)pyridin-2-yl](1-methyl-1H- indazol-6-yl)methyl]pyrrolidine-2- carboxamide |
| GZ-636 | 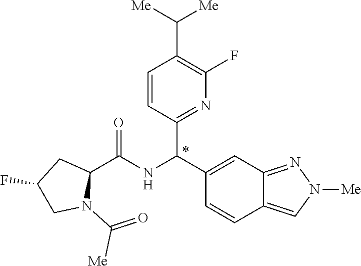 [SMILES: CC(=O)N1C[C@H](F)C[C@H]1C(=O)NC(c1ccc2cn(C)nc2c1)c1ccc(C(C)C)c(F)n1] | (2S,4R)-1-acetyl-4-fluoro-N-[(S) or (R)-[6-fluoro-5-(propan-2- yl)pyridin-2-yl](2-methyl-2H- indazol-6-yl)methyl]pyrrolidine-2- carboxamide |
| GZ-637 | 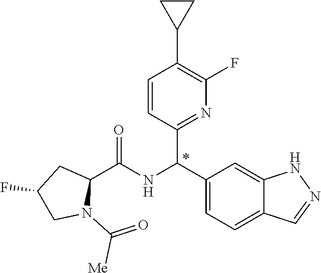 [SMILES: CC(=O)N1C[C@H](F)C[C@H]1C(=O)NC(c1ccc2cn[nH]c2c1)c1ccc(C2CC2)c(F)n1] | (2S,4R)-1-acetyl-N-[(S) or (R)-(5- cyclopropyl-6-fluoropyridin-2- yl)(1H-indazol-6-yl)methyl]-4- fluoropyrrolidine-2-carboxamide |
| GZ-638 | 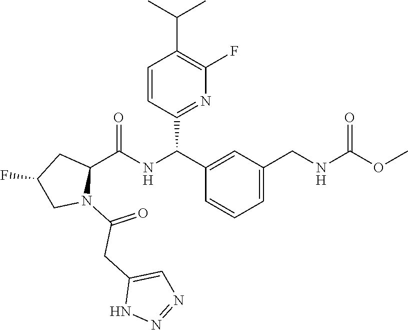 [SMILES: COC(=O)NCc1cccc([C@H](NC(=O)[C@@H]2C[C@@H](F)CN2C(=O)Cc2cnn[nH]2)c2ccc(C(C)C)c(F)n2)c1] | methyl (3-((S)-((2S,4R)-1-(2-(1H- 1,2,3-triazol-5-yl)acetyl)-4- fluoropyrrolidine-2- carboxamido)(6-fluoro-5- isopropylpyridin-2- yl)methyl)benzyl)carbamate |
| GZ-639 | 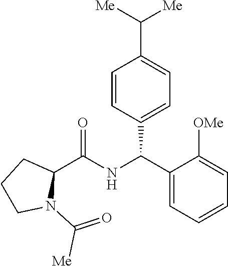 [SMILES: COc1ccccc1[C@H](NC(=O)[C@@H]1CCCN1C(C)=O)c1ccc(C(C)C)cc1] | (2S)-1-acetyl-N-[(R)-(2- methoxyphenyl)[4-(propan-2- yl)phenyl]methyl]pyrrolidine-2- carboxamide |
| GZ-640 | 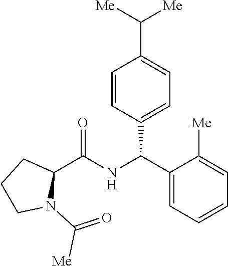 [SMILES: CC(=O)N1CCC[C@H]1C(=O)N[C@H](c1ccc(C(C)C)cc1)c1ccccc1C] | (2S)-1-acetyl-N-[(R)-(2- methylphenyl)[4-(propan-2- yl)phenyl]methyl]pyrrolidine-2- carboxamide |
| GZ-641 | 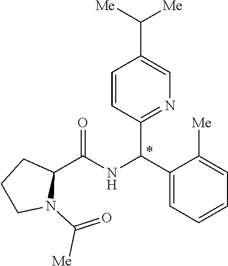 [SMILES: CC(=O)N1CCC[C@H]1C(=O)NC(c1ccc(C(C)C)cn1)c1ccccc1C] | (2S)-1-acetyl-N-[(S) or (R)-(2- methylphenyl)[5-(propan-2- yl)pyridin-2-yl]methyl]pyrrolidine- 2-carboxamide |
| GZ-642 | 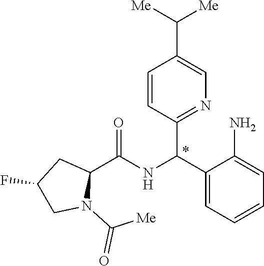 [SMILES: CC(=O)N1C[C@H](F)C[C@H]1C(=O)NC(c1ccc(C(C)C)cn1)c1ccccc1N] | (2S,4R)-1-acetyl-N-[(R) or (S)-(2- aminophenyl)[4-(propan-2- yl)phenyl]methyl]-4- fluoropyrrolidine-2-carboxamide |
| GZ-643 | 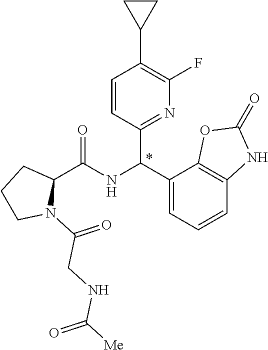 [SMILES: CC(=O)NCC(=O)N1CCC[C@H]1C(=O)NC(c1ccc(C2CC2)c(F)n1)c1cccc2[nH]c(=O)oc12] | (2S)-N-[(R) or (S)-(4-cyclopropyl- 3-fluorophenyl)(2-oxo-2,3-dihydro- 1,3-benzoxazol-7-yl)methyl]-1-(2- acetamidoacetyl)pyrrolidine-2- carboxamide |
| GZ-644 | 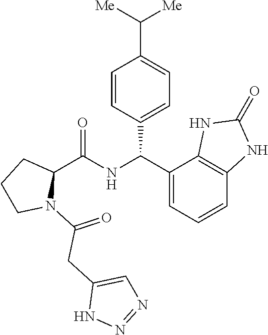 [SMILES: CC(C)c1ccc([C@@H](NC(=O)[C@@H]2CCCN2C(=O)Cc2cnn[nH]2)c2cccc3[nH]c(=O)[nH]c23)cc1] | (2S)-N-[(R)-(2-oxo-2,3-dihydro- 1H-1,3-benzodiazol-4-yl)[4- (propan-2-yl)phenyl]methyl]-1-[2- (1H-1,2,3-triazol-5- yl)acetyl]pyrrolidine-2- carboxamide |
| GZ-645 | 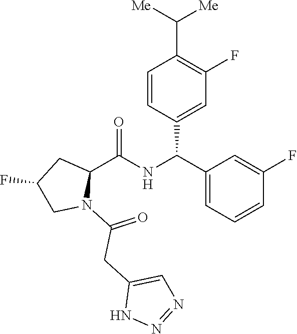 [SMILES: CC(C)c1ccc([C@@H](NC(=O)[C@@H]2C[C@@H](F)CN2C(=O)Cc2cnn[nH]2)c2cccc(F)c2)cc1F] | (2S,4R)-4-fluoro-N-[(S)-[3-fluoro- 4-(propan-2-yl)phenyl](3- fluorophenyl)methyl]-1-[2-(1H- 1,2,3-triazol-5- yl)acetyl]pyrrolidine-2- carboxamide |
| GZ-646 | 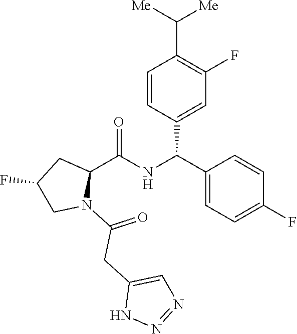 [SMILES: CC(C)c1ccc([C@@H](NC(=O)[C@@H]2C[C@@H](F)CN2C(=O)Cc2cnn[nH]2)c2ccc(F)cc2)cc1F] | (2S,4R)-4-fluoro-N-[(S)-[3-fluoro- 4-(propan-2-yl)phenyl](4- fluorophenyl)methyl]-1-[2-(1H- 1,2,3-triazol-5- yl)acetyl]pyrrolidine-2- carboxamide |
| GZ-647 | 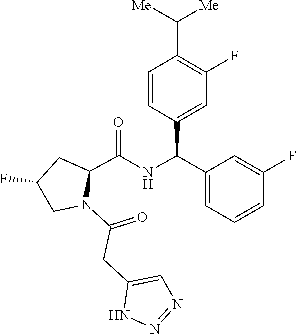 [SMILES: CC(C)c1ccc([C@H](NC(=O)[C@@H]2C[C@@H](F)CN2C(=O)Cc2cnn[nH]2)c2cccc(F)c2)cc1F] | (2S,4R)-4-fluoro-N-[(R)-[3-fluoro- 4-(propan-2-yl)phenyl](3- fluorophenyl)methyl]-1-[2-(1H- 1,2,3-triazol-5- yl)acetyl]pyrrolidine-2- carboxamide |
| GZ-648 | 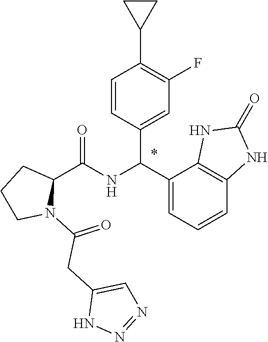 [SMILES: O=C(NC(c1ccc(C2CC2)c(F)c1)c1cccc2[nH]c(=O)[nH]c12)[C@@H]1CCCN1C(=O)Cc1cnn[nH]1] | (2S)-N-[(S)-(4-cyclopropyl-3- fluorophenyl)(2-oxo-2,3-dihydro- 1H-1,3-benzodiazol-4-yl)methyl]- 1-[2-(1H-1,2,3-triazol-5- yl)acetyl]pyrrolidine-2- carboxamide |
| GZ-649 | 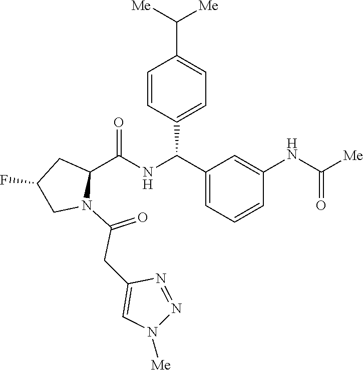 [SMILES: CC(=O)Nc1cccc([C@H](NC(=O)[C@@H]2C[C@@H](F)CN2C(=O)Cc2cn(C)nn2)c2ccc(C(C)C)cc2)c1] | (2S,4R)-N-[(R)-(3- acetamidophenyl)[4-(propan-2- yl)phenyl]methyl]-4-fluoro-1-[2-(1- methyl-1H-1,2,3-triazol-4- yl)acetyl]pyrrolidine-2- carboxamide |
| GZ-650 | 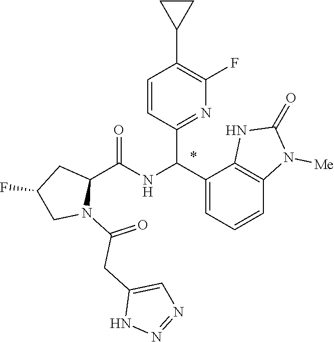 [SMILES: Cn1c(=O)[nH]c2c(C(NC(=O)[C@@H]3C[C@@H](F)CN3C(=O)Cc3cnn[nH]3)c3ccc(C4CC4)c(F)n3)cccc21] | (2S,4R)-N-[(R) or (S)-(4- cyclopropyl-3-fluorophenyl)(1- methyl-2-oxo-2,3-dihydro-1H-1,3- benzodiazol-4-yl)methyl]-4-fluoro- 1-[2-(1H-1,2,3-triazol-5- yl)acetyl]pyrrolidine-2- carboxamide |
| GZ-651 | 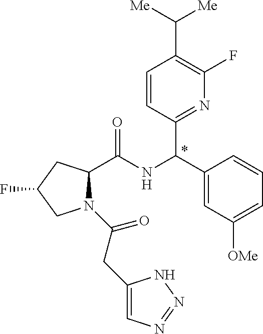 [SMILES: COc1cccc(C(NC(=O)[C@@H]2C[C@@H](F)CN2C(=O)Cc2cnn[nH]2)c2ccc(C(C)C)c(F)n2)c1] | (2S,4R)-4-fluoro-N-[(S) or (R)-[6- fluoro-5-(propan-2-yl)pyridin-2- yl](3-methoxyphenyl)methyl]-1-[2- (1H-1,2,3-triazol-5- yl)acetyl]pyrrolidine-2- carboxamide |
| GZ-652 | 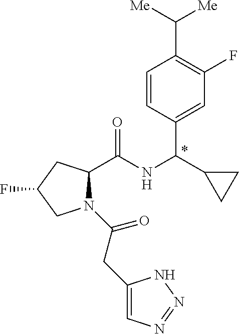 [SMILES: CC(C)c1ccc(C(NC(=O)[C@@H]2C[C@@H](F)CN2C(=O)Cc2cnn[nH]2)C2CC2)cc1F] | (2S,4R)-N-[(S) or (R)- cyclopropyl[3-fluoro-4-(propan-2- yl)phenyl]methyl]-4-fluoro-1-[2- (1H-1,2,3-triazol-5- yl)acetyl]pyrrolidine-2- carboxamide |
| GZ-653 | 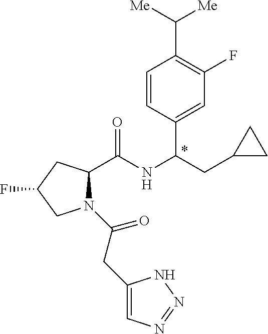 [SMILES: CC(C)c1ccc(C(CC2CC2)NC(=O)[C@@H]2C[C@@H](F)CN2C(=O)Cc2cnn[nH]2)cc1F] | (2S,4R)-N-[(1S) or (1R)-2- cyclopropyl-1-[3-fluoro-4-(propan- 2-yl)phenyl]ethyl]-4-fluoro-1-[2- (1H-1,2,3-triazol-5- yl)acetyl]pyrrolidine-2- carboxamide |
| GZ-654 | 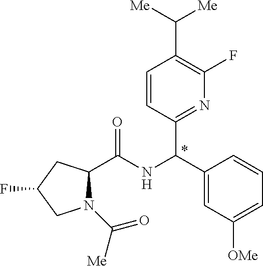 [SMILES: COc1cccc(C(NC(=O)[C@@H]2C[C@@H](F)CN2C(C)=O)c2ccc(C(C)C)c(F)n2)c1] | (2S,4R)-1-acetyl-4-fluoro-N-[(S) or (R)-[6-fluoro-5-(propan-2- yl)pyridin-2-yl](3- methoxyphenyl)methyl]pyrrolidine- 2-carboxamide |
| GZ-655 | 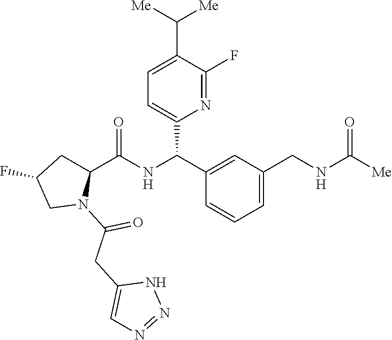 [SMILES: CC(=O)NCc1cccc([C@H](NC(=O)[C@@H]2C[C@@H](F)CN2C(=O)Cc2cnn[nH]2)c2ccc(C(C)C)c(F)n2)c1] | (2S,4R)-N-[(S)-[3- (acetamidomethyl)phenyl][6- fluoro-5-(propan-2-yl)pyridin-2- yl]methyl]-4-fluoro-1-[2-(1H-1,2,3- triazol-5-yl)acetyl]pyrrolidine-2- carboxamide |
| GZ-656 | 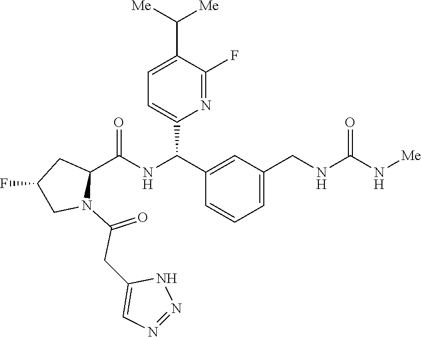 [SMILES: CNC(=O)NCc1cccc([C@H](NC(=O)[C@@H]2C[C@@H](F)CN2C(=O)Cc2cnn[nH]2)c2ccc(C(C)C)c(F)n2)c1] | (2S,4R)-4-fluoro-N-[(S)-[6-fluoro- 5-(propan-2-yl)pyridin-2-yl](3- {[(methylcarbamoyl)amino]methyl }phenyl)methyl]-1-[2-(1H-1,2,3- triazol-5-yl)acetyl]pyrrolidine-2- carboxamide |
| GZ-657 | 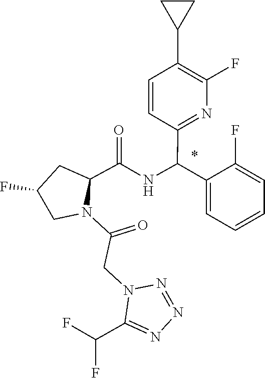 [SMILES: O=C(NC(c1ccc(C2CC2)c(F)n1)c1ccccc1F)[C@@H]1C[C@@H](F)CN1C(=O)Cn1nnnc1C(F)F] | (2S,4R)-N-[(S) or (R)-(5- cyclopropyl-6-fluoropyridin-2- yl)(2-fluorophenyl)methyl]-1-{2- [5-(difluoromethyl)-1H-1,2,3,4- tetrazol-1-yl]acetyl}-4- fluoropyrrolidine-2-carboxamide |
| GZ-658 | 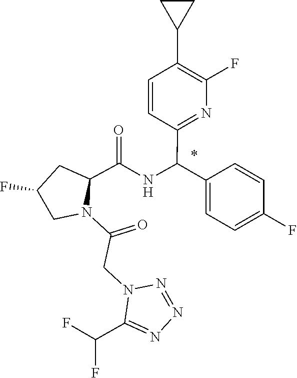 [SMILES: O=C(NC(c1ccc(F)cc1)c1ccc(C2CC2)c(F)n1)[C@@H]1C[C@@H](F)CN1C(=O)Cn1nnnc1C(F)F] | (2S,4R)-N-[(S) or (R)-(5- cyclopropyl-6-fluoropyridin-2- yl)(4-fluorophenyl)methyl]-1-{2- [5-(difluoromethyl)-1H-1,2,3,4- tetrazol-1-yl]acetyl}-4- fluoropyrrolidine-2-carboxamide |
| GZ-659 | 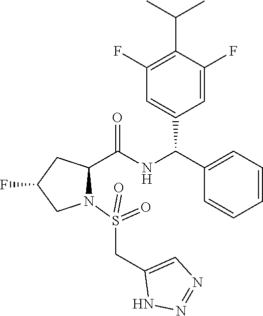 [SMILES: CC(C)c1c(F)cc([C@@H](NC(=O)[C@@H]2C[C@@H](F)CN2S(=O)(=O)Cc2cnn[nH]2)c2ccccc2)cc1F] | (2S,4R)-1-(((1H-1,2,3-triazol-5- yl)methyl)sulfonyl)-N-((S)-(3,5- difluoro-4- isopropylphenyl)(phenyl)methyl)- 4-fluoropyrrolidine-2-carboxamide |
| GZ-660 | 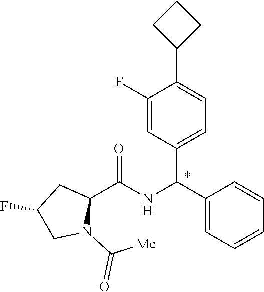 [SMILES: CC(=O)N1C[C@H](F)C[C@H]1C(=O)NC(c1ccccc1)c1ccc(C2CCC2)c(F)c1] | (2S,4R)-1-acetyl-N-[(S) or (R)-(4- cyclobutyl-3- fluorophenyl)(phenyl)methyl]-4- fluoropyrrolidine-2-carboxamide |
| GZ-661 | 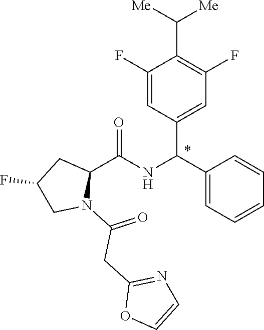 [SMILES: CC(C)c1c(F)cc(C(NC(=O)[C@@H]2C[C@@H](F)CN2C(=O)Cc2ncco2)c2ccccc2)cc1F] | (2S,4R)-N-[(S) or (R)-[3,5- difluoro-4-(propan-2- yl)phenyl](phenyl)methyl]-4- fluoro-1-[2-(1,3-oxazol-2- yl)acetyl]pyrrolidine-2- carboxamide |
| GZ-662 | 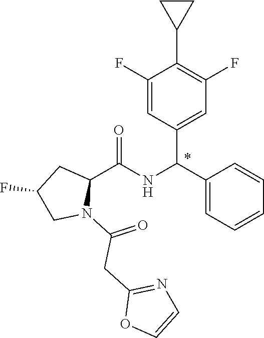 [SMILES: O=C(NC(c1ccccc1)c1cc(F)c(C2CC2)c(F)c1)[C@@H]1C[C@@H](F)CN1C(=O)Cc1ncco1] | (2S,4R)-N-[(S)-(4-cyclopropyl-3,5- difluorophenyl)(phenyl)methyl]-4- fluoro-1-[2-(1,3-oxazol-2- yl)acetyl]pyrrolidine-2- carboxamide |
| GZ-663 | 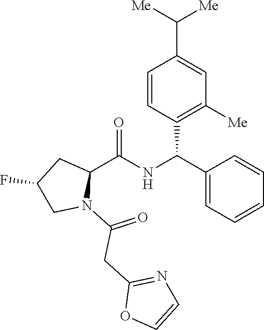 [SMILES: Cc1cc(C(C)C)ccc1[C@@H](NC(=O)[C@@H]1C[C@@H](F)CN1C(=O)Cc1ncco1)c1ccccc1] | (2S,4R)-4-fluoro-N-[(S)-[2-methyl- 4-(propan-2- yl)phenyl](phenyl)methyl]-1-[2- (1,3-oxazol-2-yl)acetyl]pyrrolidine- 2-carboxamide |
| GZ-664 | 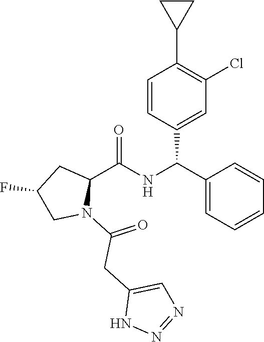 [SMILES: O=C(N[C@@H](c1ccccc1)c1ccc(C2CC2)c(Cl)c1)[C@@H]1C[C@@H](F)CN1C(=O)Cc1cnn[nH]1] | (2S,4R)-N-[(S)-(3-chloro-4- cyclopropylphenyl)(phenyl)methyl]- 4-fluoro-1-[2-(1H-1,2,3-triazol-5- yl)acetyl]pyrrolidine-2- carboxamide |
| GZ-665 | 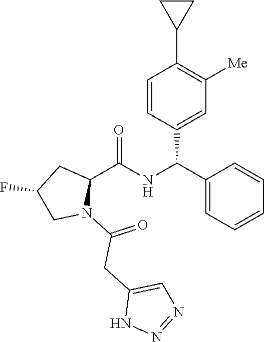 [SMILES: Cc1cc([C@@H](NC(=O)[C@@H]2C[C@@H](F)CN2C(=O)Cc2cnn[nH]2)c2ccccc2)ccc1C1CC1] | (2S,4R)-N-[(S)-(4-cyclopropyl-3- methylphenyl)(phenyl)methyl]-4- fluoro-1-[2-(1H-1,2,3-triazol-5- yl)acetyl]pyrrolidine-2- carboxamide |
| GZ-666 | 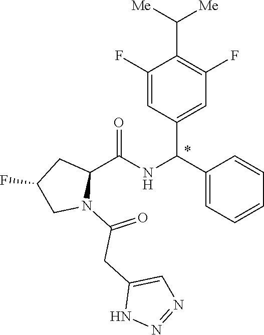 [SMILES: CC(C)c1c(F)cc(C(NC(=O)[C@@H]2C[C@@H](F)CN2C(=O)Cc2cnn[nH]2)c2ccccc2)cc1F] | (2S,4R)-N-[(R) or (S)-[3,5- difluoro-4-(propan-2- yl)phenyl](phenyl)methyl]-4- fluoro-1-[2-(1H-1,2,3-triazol-5- yl)acetyl]pyrrolidine-2- carboxamide |
| GZ-667 | 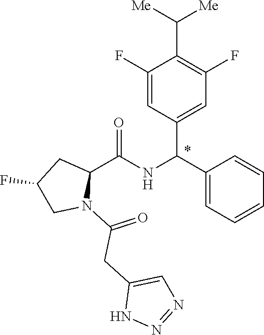 [SMILES: CC(C)c1c(F)cc(C(NC(=O)[C@@H]2C[C@@H](F)CN2C(=O)Cc2cnn[nH]2)c2ccccc2)cc1F] | (2S,4R)-N-[(S) or (R)-[3,5- difluoro-4-(propan-2- yl)phenyl](phenyl)methyl]-4- fluoro-1-[2-(1H-1,2,3-triazol-5- yl)acetyl]pyrrolidine-2- carboxamide |
| GZ-668 | 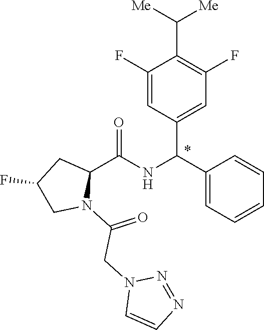 [SMILES: CC(C)c1c(F)cc(C(NC(=O)[C@@H]2C[C@@H](F)CN2C(=O)Cn2ccnn2)c2ccccc2)cc1F] | (2S,4R)-N-[(S) or (R)-[3,5- difluoro-4-(propan-2- yl)phenyl](phenyl)methyl]-4- fluoro-1-[2-(1H-1,2,3-triazol-1- yl)acetyl]pyrrolidine-2- carboxamide |
| GZ-669 | 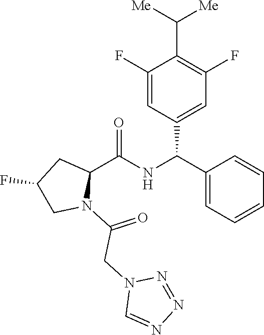 [SMILES: CC(C)c1c(F)cc([C@@H](NC(=O)[C@@H]2C[C@@H](F)CN2C(=O)Cn2cnnn2)c2ccccc2)cc1F] | (2S,4R)-N-[(S)-[3,5-difluoro-4- (propan-2- yl)phenyl](phenyl)methyl]-4- fluoro-1-[2-(1H-1,2,3,4-tetrazol-1- yl)acetyl]pyrrolidine-2- carboxamide |
| GZ-670 | 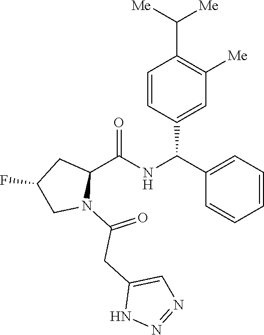 [SMILES: Cc1cc([C@@H](NC(=O)[C@@H]2C[C@@H](F)CN2C(=O)Cc2cnn[nH]2)c2ccccc2)ccc1C(C)C] | (2S,4R)-4-fluoro-N-[(S)-[3-methyl- 4-(propan-2- yl)phenyl](phenyl)methyl]-1-[2- (1H-1,2,3-triazol-5- yl)acetyl]pyrrolidine-2- carboxamide |
| GZ-671 | 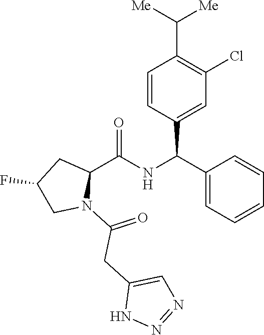 [SMILES: CC(C)c1ccc([C@H](NC(=O)[C@@H]2C[C@@H](F)CN2C(=O)Cc2cnn[nH]2)c2ccccc2)cc1Cl] | (2S,4R)-N-[(R)-[3-chloro-4- (propan-2- yl)phenyl](phenyl)methyl]-4- fluoro-1-[2-(1H-1,2,3-triazol-5- yl)acetyl]pyrrolidine-2- carboxamide |
| GZ-672 | 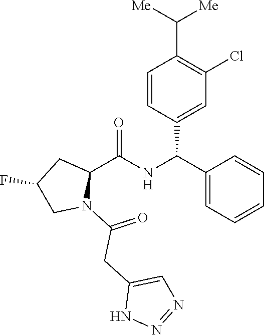 [SMILES: CC(C)c1ccc([C@@H](NC(=O)[C@@H]2C[C@@H](F)CN2C(=O)Cc2cnn[nH]2)c2ccccc2)cc1Cl] | (2S,4R)-N-[(S)-[3-chloro-4- (propan-2- yl)phenyl](phenyl)methyl]-4- fluoro-1-[2-(1H-1,2,3-triazol-5- yl)acetyl]pyrrolidine-2- carboxamide |
| GZ-673 | 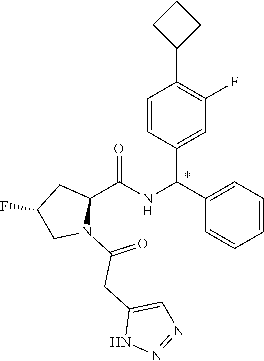 [SMILES: O=C(NC(c1ccccc1)c1ccc(C2CCC2)c(F)c1)[C@@H]1C[C@@H](F)CN1C(=O)Cc1cnn[nH]1] | (2S,4R)-N-[(R) or (S)-(4- cyclobutyl-3- fluorophenyl)(phenyl)methyl]-4- fluoro-1-[2-(1H-1,2,3-triazol-5- yl)acetyl]pyrrolidine-2- carboxamide |
| GZ-674 | 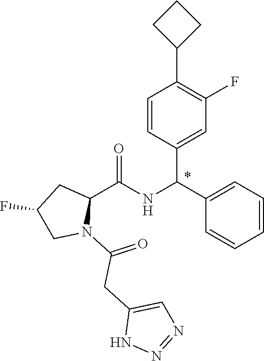 [SMILES: O=C(NC(c1ccccc1)c1ccc(C2CCC2)c(F)c1)[C@@H]1C[C@@H](F)CN1C(=O)Cc1cnn[nH]1] | (2S,4R)-N-[(S) or (R)-(4- cyclobutyl-3- fluorophenyl)(phenyl)methyl]-4- fluoro-1-[2-(1H-1,2,3-triazol-5- yl)acetyl]pyrrolidine-2- carboxamide |
| GZ-675 | 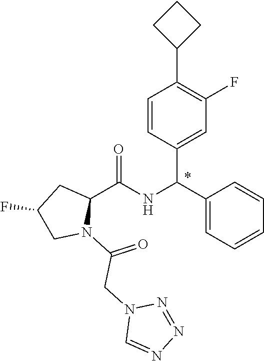 [SMILES: O=C(NC(c1ccccc1)c1ccc(C2CCC2)c(F)c1)[C@@H]1C[C@@H](F)CN1C(=O)Cn1cnnn1] | (2S,4R)-N-[(S) or (R)-(4- cyclobutyl-3- fluorophenyl)(phenyl)methyl]-4- fluoro-1-[2-(1H-1,2,3,4-tetrazol-1- yl)acetyl]pyrrolidine-2- carboxamide |
| GZ-676 | 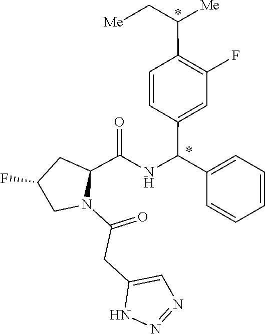 [SMILES: CCC(C)c1ccc(C(NC(=O)[C@@H]2C[C@@H](F)CN2C(=O)Cc2cnn[nH]2)c2ccccc2)cc1F] | (2S,4R)-N-[(R) or (S)-{4-[(2R) or (2S)-butan-2-yl]-3- fluorophenyl } (phenyl)methyl]-4- fluoro-1-[2-(1H-1,2,3-triazol-5- yl)acetyl]pyrrolidine-2- carboxamide |
| GZ-677 | 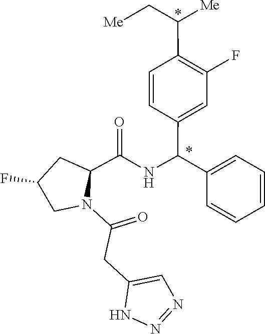 [SMILES: CCC(C)c1ccc(C(NC(=O)[C@@H]2C[C@@H](F)CN2C(=O)Cc2cnn[nH]2)c2ccccc2)cc1F] | (2S,4R)-N-[(S) or (R)-{4-[(2R) or (2S)-butan-2-yl]-3- fluorophenyl} (phenyl)methyl]-4- fluoro-1-[2-(1H-1,2,3-triazol-5- yl)acetyl]pyrrolidine-2- carboxamide |
| GZ-678 | 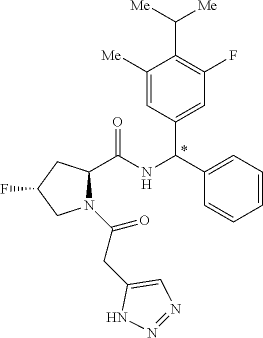 [SMILES: Cc1cc(C(NC(=O)[C@@H]2C[C@@H](F)CN2C(=O)Cc2cnn[nH]2)c2ccccc2)cc(F)c1C(C)C] | (2S,4R)-4-fluoro-N-[(R) or (S)-[3- fluoro-5-methyl-4-(propan-2- yl)phenyl](phenyl)methyl]-1-[2- (1H-1,2,3-triazol-5- yl)acetyl]pyrrolidine-2- carboxamide |
| GZ-679 | 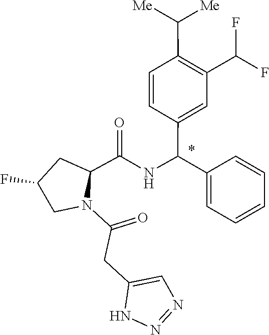 [SMILES: CC(C)c1ccc(C(NC(=O)[C@@H]2C[C@@H](F)CN2C(=O)Cc2cnn[nH]2)c2ccccc2)cc1C(F)F] | (2S,4R)-N-[(S)-[3- (difluoromethyl)-4-(propan-2- yl)phenyl](phenyl)methyl]-4- fluoro-1-[2-(1H-1,2,3-triazol-5- yl)acetyl]pyrrolidine-2- carboxamide |
| GZ-680 | 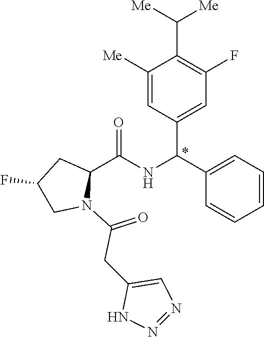 [SMILES: Cc1cc(C(NC(=O)[C@@H]2C[C@@H](F)CN2C(=O)Cc2cnn[nH]2)c2ccccc2)cc(F)c1C(C)C] | (2S,4R)-4-fluoro-N-[(S) or (R)-[3- fluoro-5-methyl-4-(propan-2- yl)phenyl](phenyl)methyl]-1-[2- (1H-1,2,3-triazol-5- yl)acetyl]pyrrolidine-2- carboxamide |
| GZ-681 | 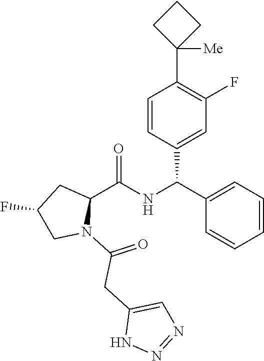 [SMILES: CC1(c2ccc([C@@H](NC(=O)[C@@H]3C[C@@H](F)CN3C(=O)Cc3cnn[nH]3)c3ccccc3)cc2F)CCC1] | (2S,4R)-4-fluoro-N-[(S)-[3-fluoro- 4-(1- methylcyclobutyl)phenyl](phenyl) methyl]-1-[2-(1H-1,2,3-triazol-5- yl)acetyl]pyrrolidine-2- carboxamide |
| GZ-682 | 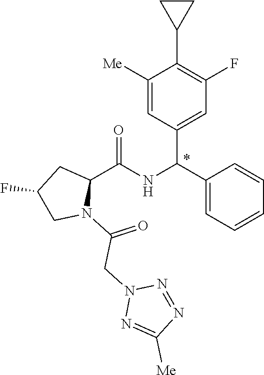 [SMILES: Cc1nnn(CC(=O)N2C[C@H](F)C[C@H]2C(=O)NC(c2ccccc2)c2cc(C)c(C3CC3)c(F)c2)n1] | (2S,4R)-N-[(S) or (R)-(4- cyclopropyl-3-fluoro-5- methylphenyl)(phenyl)methyl]-4- fluoro-1-[2-(5-methyl-2H-1,2,3,4- tetrazol-2-yl)acetyl]pyrrolidine-2- carboxamide |
| GZ-683 | 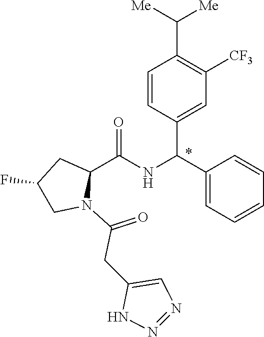 [SMILES: CC(C)c1ccc(C(NC(=O)[C@@H]2C[C@@H](F)CN2C(=O)Cc2cnn[nH]2)c2ccccc2)cc1C(F)(F)F] | (2S,4R)-4-fluoro-N-[(S) or (R)- phenyl[4-(propan-2-yl)-3- (trifluoromethyl)phenyl]methyl]-1- [2-(1H-1,2,3-triazol-5- yl)acetyl]pyrrolidine-2- carboxamide |
| GZ-684 | 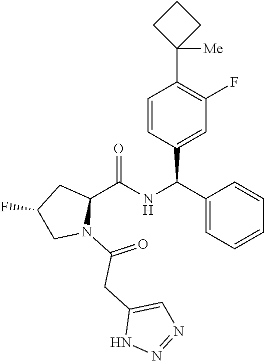 [SMILES: CC1(c2ccc([C@H](NC(=O)[C@@H]3C[C@@H](F)CN3C(=O)Cc3cnn[nH]3)c3ccccc3)cc2F)CCC1] | (2S,4R)-4-fluoro-N-[(R)-[3-fluoro- 4-(1- methylcyclobutyl)phenyl](phenyl) methyl]-1-[2-(1H-1,2,3-triazol-5- yl)acetyl]pyrrolidine-2- carboxamide |
| GZ-685 | 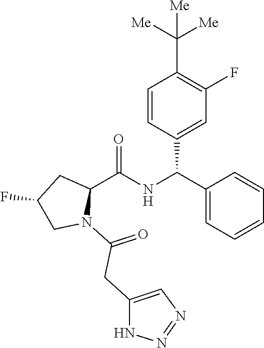 [SMILES: CC(C)(C)c1ccc([C@@H](NC(=O)[C@@H]2C[C@@H](F)CN2C(=O)Cc2cnn[nH]2)c2ccccc2)cc1F] | (2S,4R)-N-[(S)-(4-tert-butyl-3- fluorophenyl)(phenyl)methyl]-4- fluoro-1-[2-(1H-1,2,3-triazol-5- yl)acetyl]pyrrolidine-2- carboxamide |
| GZ-686 | 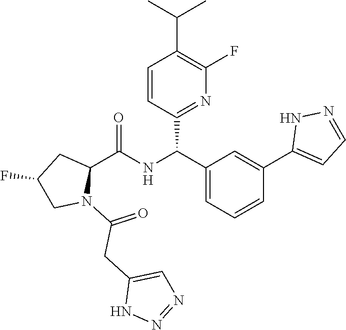 [SMILES: CC(C)c1ccc([C@@H](NC(=O)[C@@H]2C[C@@H](F)CN2C(=O)Cc2cnn[nH]2)c2cccc(-c3ccn[nH]3)c2)nc1F] | (2S,4R)-1-(2-(1H-1,2,3-triazol-5- yl)acetyl)-N-((S)-(3-(1H-pyrazol-5- yl)phenyl)(6-fluoro-5- isopropylpyridin-2-yl)methyl)-4- fluoropyrrolidine-2-carboxamide |
| GZ-687 | 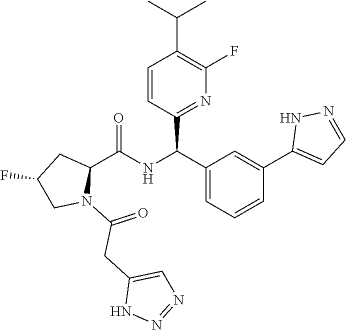 [SMILES: CC(C)c1ccc([C@H](NC(=O)[C@@H]2C[C@@H](F)CN2C(=O)Cc2cnn[nH]2)c2cccc(-c3ccn[nH]3)c2)nc1F] | (2S,4R)-1-(2-(1H-1,2,3-triazol-5- yl)acetyl)-N-((R)-(3-(1H-pyrazol-5- yl)phenyl)(6-fluoro-5- isopropylpyridin-2-yl)methyl)-4- fluoropyrrolidine-2-carboxamide |
| GZ-688 | 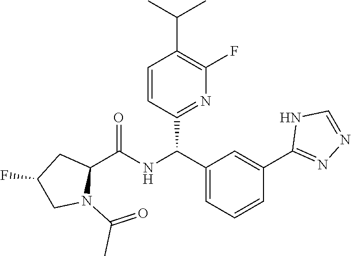 [SMILES: CC(=O)N1C[C@H](F)C[C@H]1C(=O)N[C@@H](c1cccc(-c2nnc[nH]2)c1)c1ccc(C(C)C)c(F)n1] | (2S,4R)-N-((S) or (R)-(3-(4H-1,2,4- triazol-3-yl)phenyl)(6-fluoro-5- isopropylpyridin-2-yl)methyl)-1- acetyl-4-fluoropyrrolidine-2- carboxamide |
| GZ-689 | 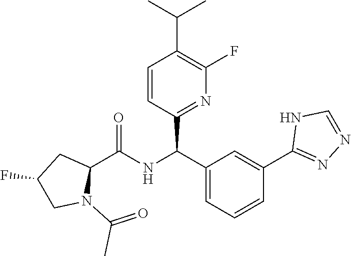 [SMILES: CC(=O)N1C[C@H](F)C[C@H]1C(=O)N[C@H](c1cccc(-c2nnc[nH]2)c1)c1ccc(C(C)C)c(F)n1] | (2S,4R)-N-((R) or (S)-(3-(4H-1,2,4- triazol-3-yl)phenyl)(6-fluoro-5- isopropylpyridin-2-yl)methyl)-1- acetyl-4-fluoropyrrolidine-2- carboxamide |
| GZ-690 | 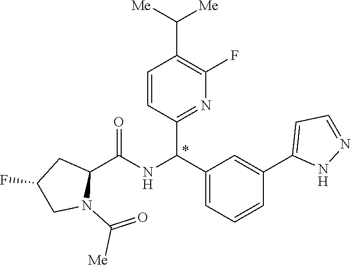 [SMILES: CC(=O)N1C[C@H](F)C[C@H]1C(=O)NC(c1cccc(-c2ccn[nH]2)c1)c1ccc(C(C)C)c(F)n1] | (2S,4R)-1-acetyl-4-fluoro-N-[(S) or (R)-[6-fluoro-5-(propan-2- yl)pyridin-2-yl][3-(1H-pyrazol-5- yl)phenyl\|methyl]pyrrolidine-2- carboxamide |
| GZ-691 | 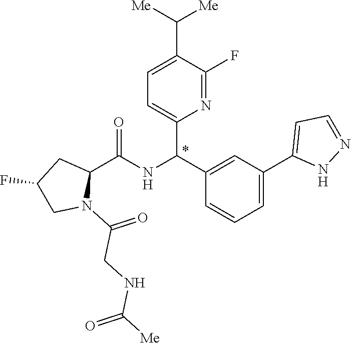 [SMILES: CC(=O)NCC(=O)N1C[C@H](F)C[C@H]1C(=O)NC(c1cccc(-c2ccn[nH]2)c1)c1ccc(C(C)C)c(F)n1] | (2S,4R)-1-(2-acetamidoacetyl)-4- fluoro-N-[(S) or (R)-[6-fluoro-5- (propan-2-yl)pyridin-2-yl][3-(1H- pyrazol-5- yl)phenyl]methyl]pyrrolidine-2- carboxamide |
| GZ-692 | 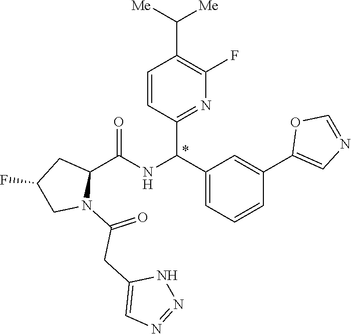 [SMILES: CC(C)c1ccc(C(NC(=O)[C@@H]2C[C@@H](F)CN2C(=O)Cc2cnn[nH]2)c2cccc(-c3cnco3)c2)nc1F] | (2S,4R)-4-fluoro-N-[(R) or (S)-[6- fluoro-5-(propan-2-yl)pyridin-2- yl][3-(1,3-oxazol-5- yl)phenyl]methyl]-1-[2-(1H-1,2,3- triazol-5-yl)acetyl]pyrrolidine-2- carboxamide |
| GZ-693 | 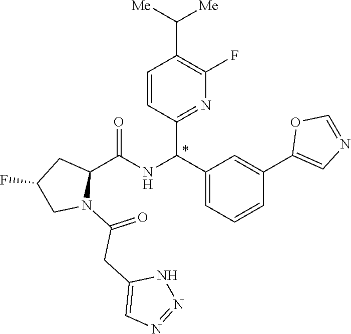 [SMILES: CC(C)c1ccc(C(NC(=O)[C@@H]2C[C@@H](F)CN2C(=O)Cc2cnn[nH]2)c2cccc(-c3cnco3)c2)nc1F] | (2S,4R)-4-fluoro-N-[(S) or (R)-[6- fluoro-5-(propan-2-yl)pyridin-2- yl][3-(1,3-oxazol-5- yl)phenyl]methyl]-1-[2-(1H-1,2,3- triazol-5-yl)acetyl]pyrrolidine-2- carboxamide |
| GZ-694 | 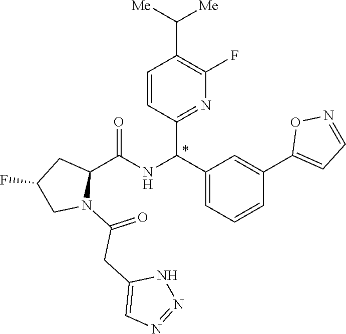 [SMILES: CC(C)c1ccc(C(NC(=O)[C@@H]2C[C@@H](F)CN2C(=O)Cc2cnn[nH]2)c2cccc(-c3ccno3)c2)nc1F] | (2S,4R)-4-fluoro-N-[(S) or (R)-[6- fluoro-5-(propan-2-yl)pyridin-2- yl][3-(1,2-oxazol-5- yl)phenyl]methyl]-1-[2-(1H-1,2,3- triazol-5-yl)acetyl]pyrrolidine-2- carboxamide |
| GZ-695 | 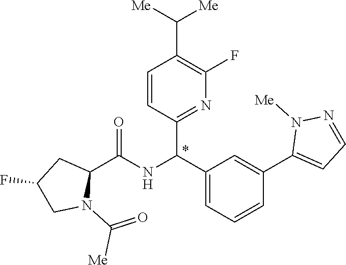 [SMILES: CC(=O)N1C[C@H](F)C[C@H]1C(=O)NC(c1cccc(-c2ccnn2C)c1)c1ccc(C(C)C)c(F)n1] | (2S,4R)-1-acetyl-4-fluoro-N-[(S) or (R)-[6-fluoro-5-(propan-2- yl)pyridin-2-yl][3-(1-methyl-1H- pyrazol-5- yl)phenyl methyl]pyrrolidine-2- carboxamide |
| GZ-696 | 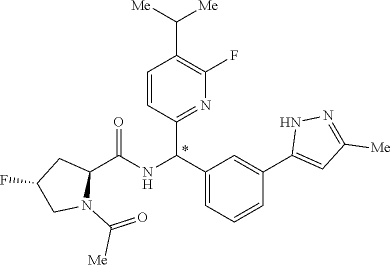 [SMILES: CC(=O)N1C[C@H](F)C[C@H]1C(=O)NC(c1cccc(-c2cc(C)n[nH]2)c1)c1ccc(C(C)C)c(F)n1] | (2S,4R)-1-acetyl-4-fluoro-N-[(S) or (R)-[6-fluoro-5-(propan-2- yl)pyridin-2-yl][3-(3-methyl-1H- pyrazol-5- yl)phenyl]methyl]pyrrolidine-2- carboxamide |
| GZ-697 | 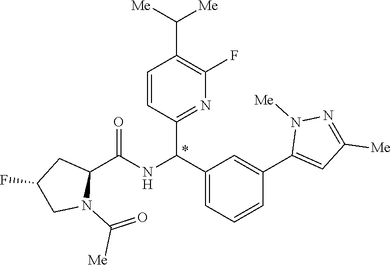 [SMILES: CC(=O)N1C[C@H](F)C[C@H]1C(=O)NC(c1cccc(-c2cc(C)nn2C)c1)c1ccc(C(C)C)c(F)n1] | (2S,4R)-1-acetyl-N-[(S) or (R)-[3- (1,3-dimethyl-1H-pyrazol-5- yl)phenyl][6-fluoro-5-(propan-2- yl)pyridin-2-yl]methyl]-4- fluoropyrrolidine-2-carboxamide |
| GZ-698 | 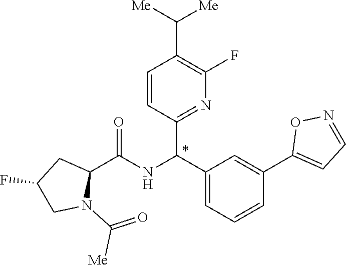 [SMILES: CC(=O)N1C[C@H](F)C[C@H]1C(=O)NC(c1cccc(-c2ccno2)c1)c1ccc(C(C)C)c(F)n1] | (2S,4R)-1-acetyl-4-fluoro-N-[(S) or (R)-[6-fluoro-5-(propan-2- yl)pyridin-2-yl][3-(1,2-oxazol-5- yl)phenyl]methyl]pyrrolidine-2- carboxamide |
| GZ-699 | 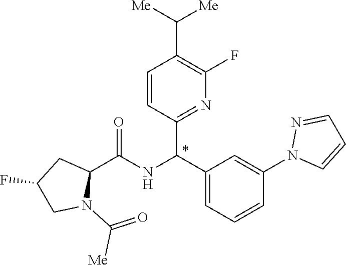 [SMILES: CC(=O)N1C[C@H](F)C[C@H]1C(=O)NC(c1cccc(-n2cccn2)c1)c1ccc(C(C)C)c(F)n1] | (2S,4R)-1-acetyl-4-fluoro-N-[(S) or (R)-[6-fluoro-5-(propan-2- yl)pyridin-2-yl][3-(1H-pyrazol-1- yl)phenyl]methyl]pyrrolidine-2- carboxamide |
| GZ-700 | 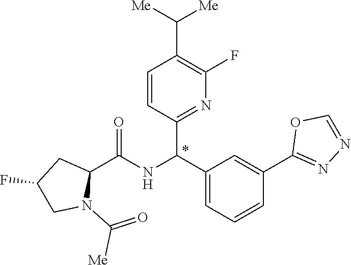 [SMILES: CC(=O)N1C[C@H](F)C[C@H]1C(=O)NC(c1cccc(-c2nnco2)c1)c1ccc(C(C)C)c(F)n1] | (2S,4R)-1-acetyl-4-fluoro-N-[(S) or (R)-[6-fluoro-5-(propan-2- yl)pyridin-2-yl][3-(1,3,4-oxadiazol- 2-yl)phenyl]methyl]pyrrolidine-2- carboxamide |
| GZ-701 | 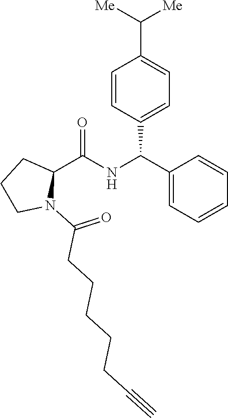 [SMILES: C#CCCCCCC(=O)N1CCC[C@H]1C(=O)N[C@@H](c1ccccc1)c1ccc(C(C)C)cc1] | (2S)-1-(oct-7-ynoyl)-N-[(S)- phenyl[4-(propan-2- yl)phenyl]methyl]pyrrolidine-2- carboxamide |
| GZ-702 | 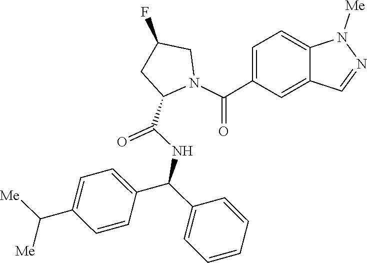 [SMILES: CC(C)c1ccc([C@@H](NC(=O)[C@@H]2C[C@@H](F)CN2C(=O)c2ccc3c(cnn3C)c2)c2ccccc2)cc1] | (2S,4R)-4-fluoro-1-(1-methyl-1H- indazole-5-carbonyl)-N-[(S)- phenyl[4-(propan-2- yl)phenyl methyl]pyrrolidine-2- carboxamide |
| GZ-703 | 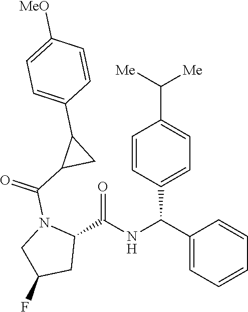 [SMILES: COc1ccc(C2CC2C(=O)N2C[C@H](F)C[C@H]2C(=O)N[C@@H](c2ccccc2)c2ccc(C(C)C)cc2)cc1] | (2S,4R)-4-fluoro-1-[2-(4- methoxyphenyl)cyclopropanecarbonyl]- N-[(S)-phenyl[4-(propan-2- yl)phenyl]methyl]pyrrolidine-2- carboxamide |
| GZ-704 | 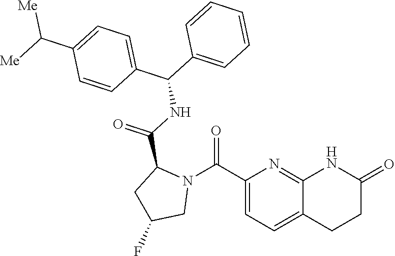 [SMILES: CC(C)c1ccc([C@@H](NC(=O)[C@@H]2C[C@@H](F)CN2C(=O)c2ccc3c(n2)NC(=O)CC3)c2ccccc2)cc1] | (2S,4R)-4-fluoro-1-(7-oxo-5,6,7,8- tetrahydro-1,8-naphthyridine-2- carbonyl)-N-[(S)-phenyl[4-(propan- 2-yl)phenyl]methyl]pyrrolidine-2- carboxamide |
| GZ-705 | 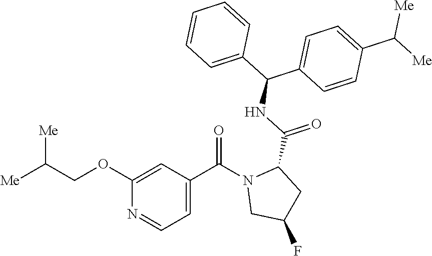 [SMILES: CC(C)COc1cc(C(=O)N2C[C@H](F)C[C@H]2C(=O)N[C@@H](c2ccccc2)c2ccc(C(C)C)cc2)ccn1] | (2S,4R)-4-fluoro-1-[2-(2- methylpropoxy )pyridine-4- carbonyl]-N-[(S)-phenyl[4-(propan- 2-yl)phenyl]methyl]pyrrolidine-2- carboxamide |
| GZ-707 | 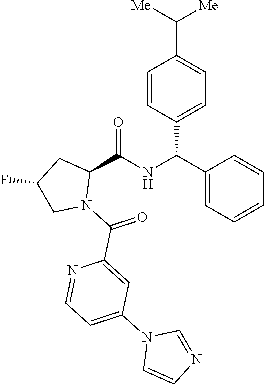 [SMILES: CC(C)c1ccc([C@@H](NC(=O)[C@@H]2C[C@@H](F)CN2C(=O)c2cc(-n3ccnc3)ccn2)c2ccccc2)cc1] | (2S,4R)-4-fluoro-1-[4-(1H- imidazol-1-yl)pyridine-2-carbonyl]- N-[(S)-phenyl[4-(propan-2- yl)phenyl]methyl]pyrrolidine-2- carboxamide |
| GZ-708 | 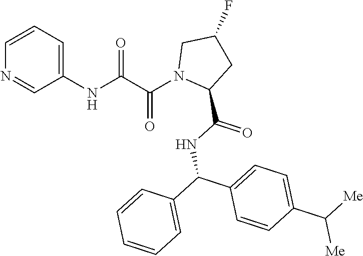 [SMILES: CC(C)c1ccc([C@@H](NC(=O)[C@@H]2C[C@@H](F)CN2C(=O)C(=O)Nc2cccnc2)c2ccccc2)cc1] | (2S,4R)-4-fluoro-N-[(S)-phenyl[4- (propan-2-yl)phenyl]methyl]-1- {[(pyridin-3- yl) carbamoyl\| carbonyl } pyrrolidine- 2-carboxamide |
| GZ-709 | 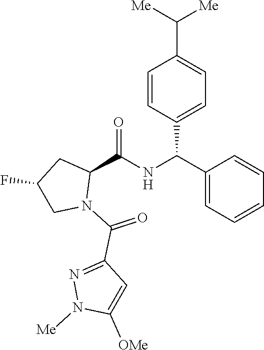 [SMILES: COc1cc(C(=O)N2C[C@H](F)C[C@H]2C(=O)N[C@@H](c2ccccc2)c2ccc(C(C)C)cc2)nn1C] | (2S,4R)-4-fluoro-1-(5-methoxy-1- methyl-1H-pyrazole-3-carbonyl)- N-[(S)-phenyl[4-(propan-2- yl)phenyl]methyl]pyrrolidine-2- carboxamide |
| GZ-710 | 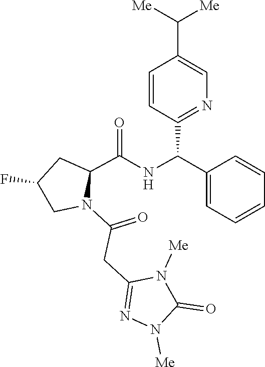 [SMILES: CC(C)c1ccc([C@@H](NC(=O)[C@@H]2C[C@@H](F)CN2C(=O)Cc2nn(C)c(=O)n2C)c2ccccc2)nc1] | (2S,4R)-1-[2-(1,4-dimethyl-5-oxo- 4,5-dihydro-1H-1,2,4-triazol-3- yl)acetyl]-4-fluoro-N-[(S)- phenyl[5-(propan-2-yl)pyridin-2- yl]methyl]pyrrolidine-2- carboxamide |
| GZ-711 | 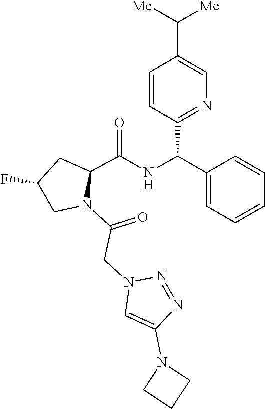 [SMILES: CC(C)c1ccc([C@@H](NC(=O)[C@@H]2C[C@@H](F)CN2C(=O)Cn2cc(N3CCC3)nn2)c2ccccc2)nc1] | (2S,4R)-1-{2-[4-(azetidin-1-yl)-1H- 1,2,3-triazol-1-yl]acetyl}-4-fluoro- N-[(S)-phenyl[5-(propan-2- yl)pyridin-2-yl]methyl]pyrrolidine- 2-carboxamide |
| GZ-712 | 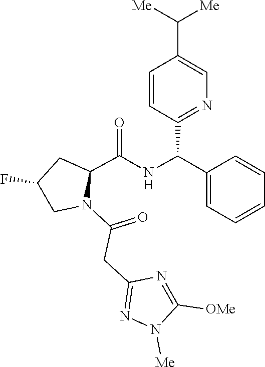 [SMILES: COc1nc(CC(=O)N2C[C@H](F)C[C@H]2C(=O)N[C@@H](c2ccccc2)c2ccc(C(C)C)cn2)nn1C] | (2S,4R)-4-fluoro-1-[2-(5-methoxy- 1-methyl-1H-1,2,4-triazol-3- yl)acetyl]-N-[(S)-phenyl[5-(propan- 2-yl)pyridin-2- yl]methyl]pyrrolidine-2- carboxamide |
| GZ-713 | 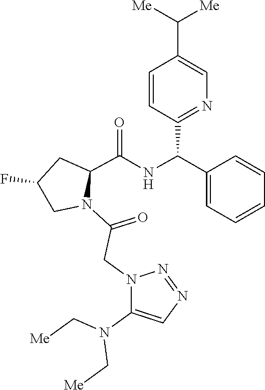 [SMILES: CCN(CC)c1cnnn1CC(=O)N1C[C@H](F)C[C@H]1C(=O)N[C@@H](c1ccccc1)c1ccc(C(C)C)cn1] | (2S,4R)-1-{2-[5-(diethylamino)- 1H-1,2,3-triazol-1-yl]acetyl}-4- fluoro-N-[(S)-phenyl[5-(propan-2- yl)pyridin-2-yl]methyl]pyrrolidine- 2-carboxamide |
| GZ-714 | 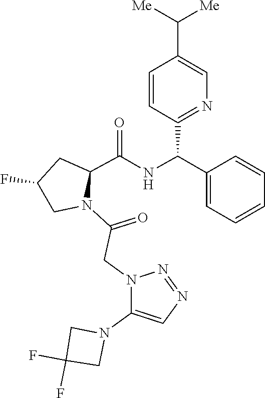 [SMILES: CC(C)c1ccc([C@@H](NC(=O)[C@@H]2C[C@@H](F)CN2C(=O)Cn2nncc2N2CC(F)(F)C2)c2ccccc2)nc1] | (2S,4R)-1-{2-[5-(3,3- difluoroazetidin-1-yl)-1H-1,2,3- triazol-1-yl]acetyl }-4-fluoro-N- [(S)-phenyl[5-(propan-2-yl)pyridin- 2-yl]methyl]pyrrolidine-2- carboxamide |
| GZ-715 | 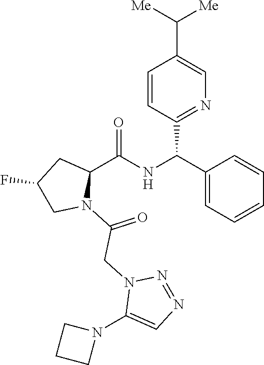 [SMILES: CC(C)c1ccc([C@@H](NC(=O)[C@@H]2C[C@@H](F)CN2C(=O)Cn2nncc2N2CCC2)c2ccccc2)nc1] | (2S,4R)-1-{2-[5-(azetidin-1-yl)-1H- 1,2,3-triazol-1-yl]acetyl}-4-fluoro- N-[(S)-phenyl[5-(propan-2- yl)pyridin-2-yl\|methyl]pyrrolidine- 2-carboxamide |
| GZ-716 | 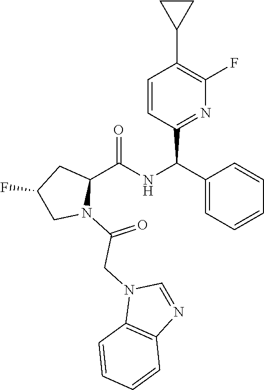 [SMILES: O=C(N[C@H](c1ccccc1)c1ccc(C2CC2)c(F)n1)[C@@H]1C[C@@H](F)CN1C(=O)Cn1cnc2ccccc21] | (2S,4R)-1-[2-(1H-1,3-benzodiazol- 1-yl)acetyl]-N-[(R)-(5-cyclopropyl- 6-fluoropyridin-2- yl)(phenyl)methyl]-4- fluoropyrrolidine-2-carboxamide |
| GZ-717 | 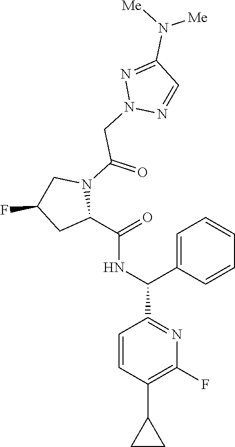 [SMILES: CN(C)c1cnn(CC(=O)N2C[C@H](F)C[C@H]2C(=O)N[C@H](c2ccccc2)c2ccc(C3CC3)c(F)n2)n1] | (2S,4R)-N-[(R)-(5-cyclopropyl-6- fluoropyridin-2-yl)(phenyl)methyl]- 1-{2-[4-(dimethylamino)-2H-1,2,3- triazol-2-yl]acetyl}-4- fluoropyrrolidine-2-carboxamide |
| GZ-718 | 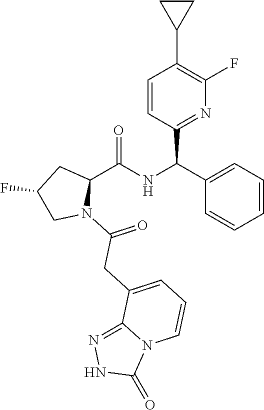 [SMILES: O=C(N[C@H](c1ccccc1)c1ccc(C2CC2)c(F)n1)[C@@H]1C[C@@H](F)CN1C(=O)Cc1cccn2c(=O)[nH]nc12] | (2S,4R)-N-[(R)-(5-cyclopropyl-6- fluoropyridin-2-yl)(phenyl)methyl]- 4-fluoro-1-(2-{3-oxo-2H,3H- [1,2,4]triazolo[4,3-a]pyridin-8- yl}acetyl)pyrrolidine-2- carboxamide |
| GZ-719 | 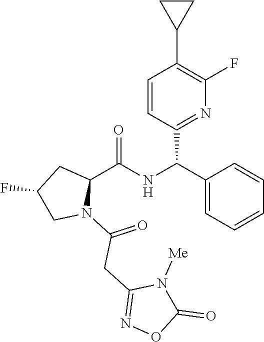 [SMILES: Cn1c(CC(=O)N2C[C@H](F)C[C@H]2C(=O)N[C@@H](c2ccccc2)c2ccc(C3CC3)c(F)n2)noc1=O] | (2S,4R)-N-[(S)-(5-cyclopropyl-6- fluoropyridin-2-yl)(phenyl)methyl]- 4-fluoro-1-[2-(4-methyl-5-oxo-4,5- dihydro-1,2,4-oxadiazol-3- yl)acetyl]pyrrolidine-2- carboxamide |
| GZ-720 | 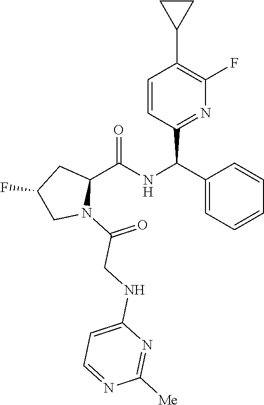 [SMILES: Cc1nccc(NCC(=O)N2C[C@H](F)C[C@H]2C(=O)N[C@H](c2ccccc2)c2ccc(C3CC3)c(F)n2)n1] | (2S,4R)-N-[(R)-(5-cyclopropyl-6- fluoropyridin-2-yl)(phenyl)methyl]- 4-fluoro-1-{2-[(2-methylpyrimidin- 4-yl)amino]acetyl } pyrrolidine-2- carboxamide |
| GZ-721 | 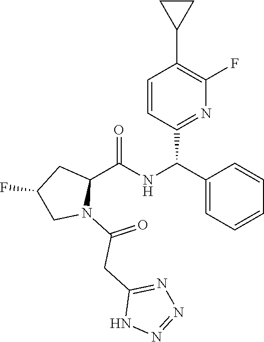 [SMILES: O=C(N[C@@H](c1ccccc1)c1ccc(C2CC2)c(F)n1)[C@@H]1C[C@@H](F)CN1C(=O)Cc1nnn[nH]1] | (2S,4R)-N-[(S)-(5-cyclopropyl-6- fluoropyridin-2-yl)(phenyl)methyl]- 4-fluoro-1-[2-(1H-1,2,3-triazol-5- yl)acetyl]pyrrolidine-2- carboxamide |
| GZ-722 | 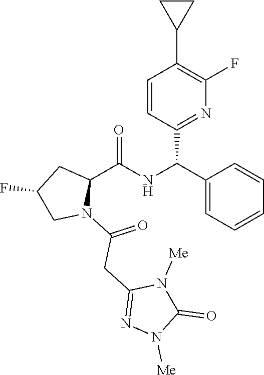 [SMILES: Cn1nc(CC(=O)N2C[C@H](F)C[C@H]2C(=O)N[C@@H](c2ccccc2)c2ccc(C3CC3)c(F)n2)n(C)c1=O] | (2S,4R)-N-[(S)-(5-cyclopropyl-6- fluoropyridin-2-yl)(phenyl)methyl]- 1-[2-(1,4-dimethyl-5-oxo-4,5- dihydro-1H-1,2,4-triazol-3- yl)acetyl]-4-fluoropyrrolidine-2- carboxamide |
| GZ-723 | 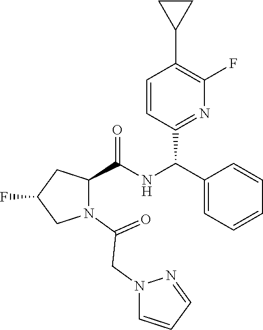 [SMILES: O=C(N[C@@H](c1ccccc1)c1ccc(C2CC2)c(F)n1)[C@@H]1C[C@@H](F)CN1C(=O)Cn1cccn1] | (2S,4R)-N-[(S)-(5-cyclopropyl-6- fluoropyridin-2-yl)(phenyl)methyl]- 4-fluoro-1-[2-(1H-pyrazol-1- yl)acetyl]pyrrolidine-2- carboxamide |
| GZ-724 | 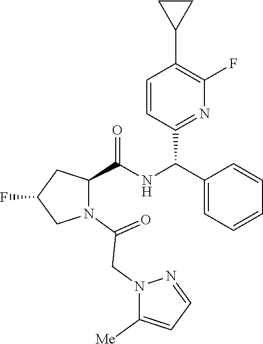 [SMILES: Cc1ccnn1CC(=O)N1C[C@H](F)C[C@H]1C(=O)N[C@@H](c1ccccc1)c1ccc(C2CC2)c(F)n1] | (2S,4R)-N-[(S)-(5-cyclopropyl-6- fluoropyridin-2-yl)(phenyl)methyl]- 4-fluoro-1-[2-(5-methyl-1H- pyrazol-1-yl)acetyl]pyrrolidine-2- carboxamide |
| GZ-725 | 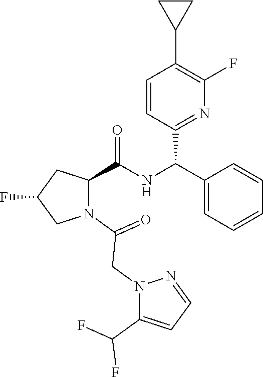 [SMILES: O=C(N[C@@H](c1ccccc1)c1ccc(C2CC2)c(F)n1)[C@@H]1C[C@@H](F)CN1C(=O)Cn1nccc1C(F)F] | (2S,4R)-N-[(S)-(5-cyclopropyl-6- fluoropyridin-2-yl)(phenyl)methyl]- 1-{2-[5-(difluoromethyl)-1H- pyrazol-1-yl]acetyl}-4- fluoropyrrolidine-2-carboxamide |
| GZ-726 | 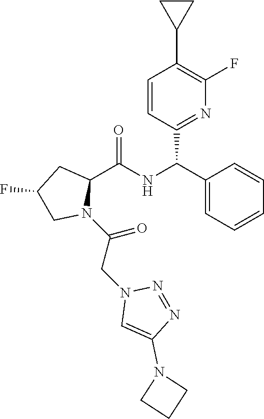 [SMILES: O=C(N[C@@H](c1ccccc1)c1ccc(C2CC2)c(F)n1)[C@@H]1C[C@@H](F)CN1C(=O)Cn1cc(N2CCC2)nn1] | (2S,4R)-1-{2-[4-(azetidin-1-yl)-1H- 1,2,3-triazol-1-yl]acetyl}-N-[(S)-(5- cyclopropyl-6-fluoropyridin-2- yl)(phenyl)methyl]-4- fluoropyrrolidine-2-carboxamide |
| GZ-727 | 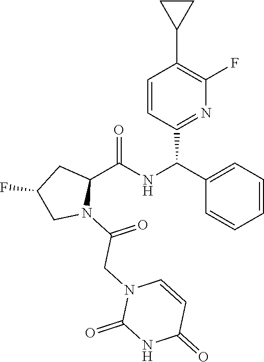 [SMILES: O=C(N[C@@H](c1ccccc1)c1ccc(C2CC2)c(F)n1)[C@@H]1C[C@@H](F)CN1C(=O)Cn1ccc(=O)[nH]c1=O] | (2S,4R)-N-[(S)-(5-cyclopropyl-6- fluoropyridin-2-yl)(phenyl)methyl]- 1-[2-(2,4-dioxo-1,2,3,4- tetrahydropyrimidin-1-yl)acetyl]-4- fluoropyrrolidine-2-carboxamide |
| GZ-728 | 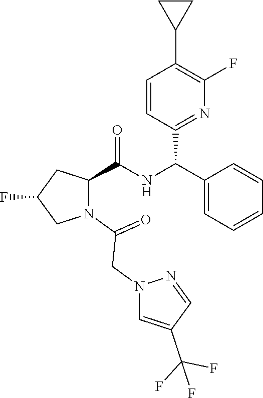 [SMILES: O=C(N[C@@H](c1ccccc1)c1ccc(C2CC2)c(F)n1)[C@@H]1C[C@@H](F)CN1C(=O)Cn1cc(C(F)(F)F)cn1] | (2S,4R)-N-[(S)-(5-cyclopropyl-6- fluoropyridin-2-yl)(phenyl)methyl]- 4-fluoro-1-{2-[4-(trifluoromethyl)- 1H-pyrazol-1-yl]acetyl} pyrrolidine- 2-carboxamide |
| GZ-729 | 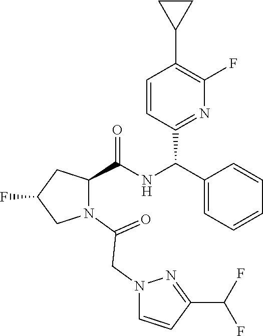 [SMILES: O=C(N[C@@H](c1ccccc1)c1ccc(C2CC2)c(F)n1)[C@@H]1C[C@@H](F)CN1C(=O)Cn1ccc(C(F)F)n1] | (2S,4R)-N-[(S)-(5-cyclopropyl-6- fluoropyridin-2-yl)(phenyl)methyl]- 1-{2-[3-(difluoromethyl)-1H- pyrazol-1-yl]acetyl}-4- fluoropyrrolidine-2-carboxamide |
| GZ-730 | 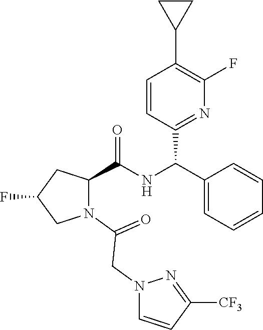 [SMILES: O=C(N[C@@H](c1ccccc1)c1ccc(C2CC2)c(F)n1)[C@@H]1C[C@@H](F)CN1C(=O)Cn1ccc(C(F)(F)F)n1] | (2S,4R)-N-[(S)-(5-cyclopropyl-6- fluoropyridin-2-yl)(phenyl)methyl]- 4-fluoro-1-{2-[3-(trifluoromethyl)- 1H-pyrazol-1-yl]acetyl} pyrrolidine- 2-carboxamide |
| GZ-731 | 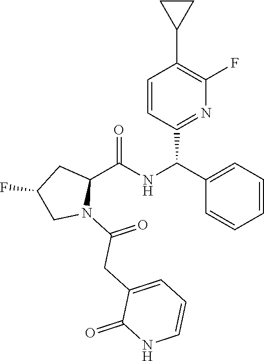 [SMILES: O=C(N[C@@H](c1ccccc1)c1ccc(C2CC2)c(F)n1)[C@@H]1C[C@@H](F)CN1C(=O)Cc1ccc[nH]c1=O] | (2S,4R)-N-[(S)-(5-cyclopropyl-6- fluoropyridin-2-yl)(phenyl)methyl]- 4-fluoro-1-[2-(2-oxo-1,2- dihydropyridin-3- yl)acetyl]pyrrolidine-2- carboxamide |
| GZ-732 | 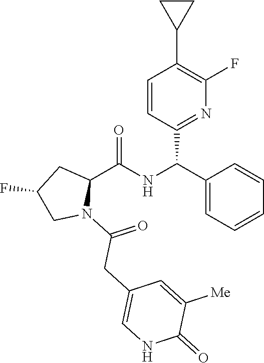 [SMILES: Cc1cc(CC(=O)N2C[C@H](F)C[C@H]2C(=O)N[C@@H](c2ccccc2)c2ccc(C3CC3)c(F)n2)c[nH]c1=O] | (2S,4R)-N-[(S)-(5-cyclopropyl-6- fluoropyridin-2-yl)(phenyl)methyl]- 4-fluoro-1-[2-(5-methyl-6-oxo-1,6- dihydropyridin-3- yl)acetyl]pyrrolidine-2- carboxamide |
| GZ-733 | 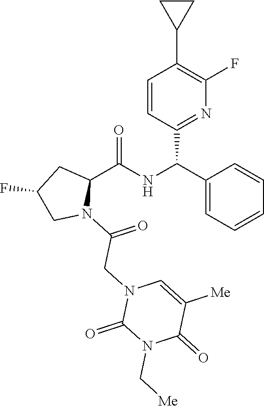 [SMILES: CCn1c(=O)c(C)cn(CC(=O)N2C[C@H](F)C[C@H]2C(=O)N[C@@H](c2ccccc2)c2ccc(C3CC3)c(F)n2)c1=O] | (2S,4R)-N-[(S)-(5-cyclopropyl-6- fluoropyridin-2-yl)(phenyl)methyl]- 1-[2-(3-ethyl-5-methyl-2,4-dioxo- 1,2,3,4-tetrahydropyrimidin-1- yl)acetyl]-4-fluoropyrrolidine-2- carboxamide |
| GZ-734 | 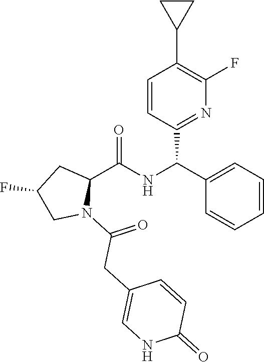 [SMILES: O=C(N[C@@H](c1ccccc1)c1ccc(C2CC2)c(F)n1)[C@@H]1C[C@@H](F)CN1C(=O)Cc1ccc(=O)[nH]c1] | (2S,4R)-N-[(S)-(5-cyclopropyl-6- fluoropyridin-2-yl)(phenyl)methyl]- 4-fluoro-1-[2-(6-oxo-1,6- dihydropyridin-3- yl)acetyl]pyrrolidine-2- carboxamide |
| GZ-735 | 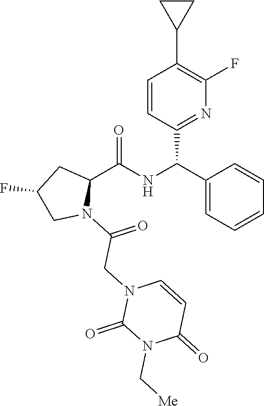 [SMILES: CCn1c(=O)ccn(CC(=O)N2C[C@H](F)C[C@H]2C(=O)N[C@@H](c2ccccc2)c2ccc(C3CC3)c(F)n2)c1=O] | (2S,4R)-N-[(S)-(5-cyclopropyl-6- fluoropyridin-2-yl)(phenyl)methyl]- 1-[2-(3-ethyl-2,4-dioxo-1,2,3,4- tetrahydropyrimidin-1-yl)acetyl]-4- fluoropyrrolidine-2-carboxamide |
| GZ-736 | 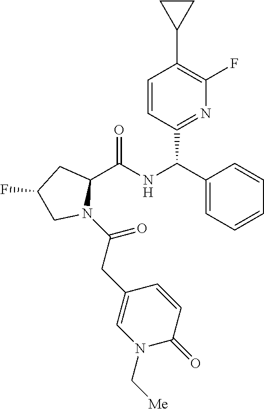 [SMILES: CCn1cc(CC(=O)N2C[C@H](F)C[C@H]2C(=O)N[C@@H](c2ccccc2)c2ccc(C3CC3)c(F)n2)ccc1=O] | (2S,4R)-N-[(S)-(5-cyclopropyl-6- fluoropyridin-2-yl)(phenyl)methyl]- 1-[2-(1-ethyl-6-oxo-1,6- dihydropyridin-3-yl)acetyl]-4- fluoropyrrolidine-2-carboxamide |
| GZ-737 | 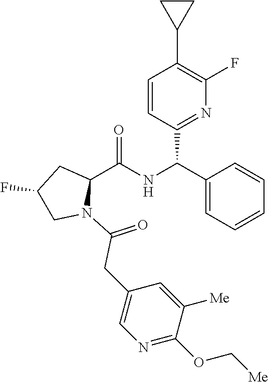 [SMILES: CCOc1ncc(CC(=O)N2C[C@H](F)C[C@H]2C(=O)N[C@@H](c2ccccc2)c2ccc(C3CC3)c(F)n2)cc1C] | (2S,4R)-N-[(S)-(5-cyclopropyl-6- fluoropyridin-2-yl)(phenyl)methyl]- 1-[2-(6-ethoxy-5-methylpyridin-3- yl)acetyl]-4-fluoropyrrolidine-2- carboxamide |
| GZ-738 | 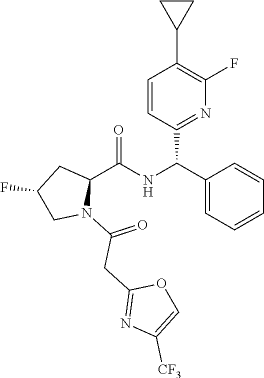 [SMILES: O=C(N[C@@H](c1ccccc1)c1ccc(C2CC2)c(F)n1)[C@@H]1C[C@@H](F)CN1C(=O)Cc1nc(C(F)(F)F)co1] | (2S,4R)-N-[(S)-(5-cyclopropyl-6- fluoropyridin-2-yl)(phenyl)methyl]- 4-fluoro-1-{2-[4-(trifluoromethyl)- 1,3-oxazol-2-yl]acetyl} pyrrolidine- 2-carboxamide |
| GZ-739 | 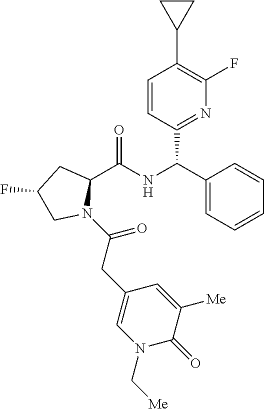 [SMILES: CCn1cc(CC(=O)N2C[C@H](F)C[C@H]2C(=O)N[C@@H](c2ccccc2)c2ccc(C3CC3)c(F)n2)cc(C)c1=O] | (2S,4R)-N-[(S)-(5-cyclopropyl-6- fluoropyridin-2-yl)(phenyl)methyl]- 1-[2-(1-ethyl-5-methyl-6-oxo-1,6- dihydropyridin-3-yl)acetyl]-4- fluoropyrrolidine-2-carboxamide |
| GZ-740 | 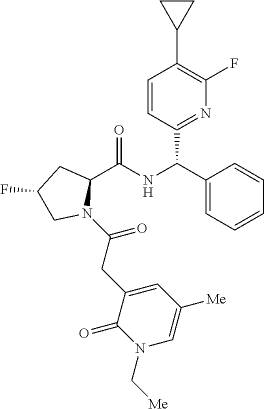 [SMILES: CCn1cc(C)cc(CC(=O)N2C[C@H](F)C[C@H]2C(=O)N[C@@H](c2ccccc2)c2ccc(C3CC3)c(F)n2)c1=O] | (2S,4R)-N-[(S)-(5-cyclopropyl-6- fluoropyridin-2-yl)(phenyl)methyl]- 1-[2-(1-ethyl-5-methyl-2-oxo-1,2- dihydropyridin-3-yl)acetyl]-4- fluoropyrrolidine-2-carboxamide |
| GZ-741 | 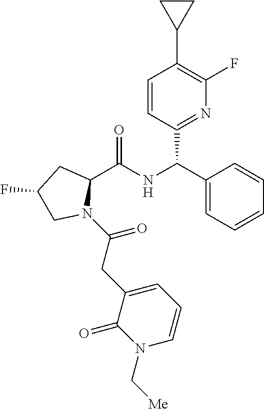 [SMILES: CCn1cccc(CC(=O)N2C[C@H](F)C[C@H]2C(=O)N[C@@H](c2ccccc2)c2ccc(C3CC3)c(F)n2)c1=O] | (2S,4R)-N-[(S)-(5-cyclopropyl-6- fluoropyridin-2-yl)(phenyl)methyl]- 1-[2-(1-ethyl-2-oxo-1,2- dihydropyridin-3-yl)acetyl]-4- fluoropyrrolidine-2-carboxamide |
| GZ-742 | 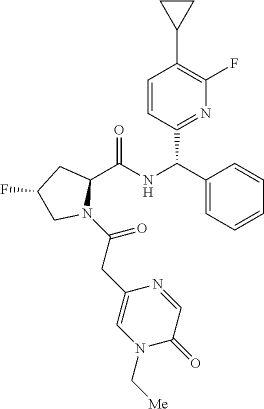 [SMILES: CCn1cc(CC(=O)N2C[C@H](F)C[C@H]2C(=O)N[C@@H](c2ccccc2)c2ccc(C3CC3)c(F)n2)ncc1=O] | (2S,4R)-N-[(S)-(5-cyclopropyl-6- fluoropyridin-2-yl)(phenyl)methyl]- 1-[2-(4-ethyl-5-oxo-4,5- dihydropyrazin-2-yl)acetyl]-4- fluoropyrrolidine-2-carboxamide |
| GZ-743 | 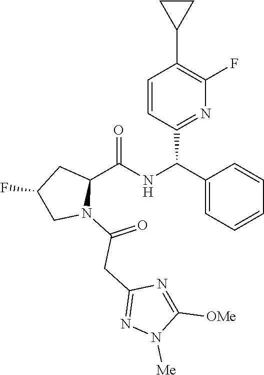 [SMILES: COc1nc(CC(=O)N2C[C@H](F)C[C@H]2C(=O)N[C@@H](c2ccccc2)c2ccc(C3CC3)c(F)n2)nn1C] | (2S,4R)-N-[(S)-(5-cyclopropyl-6- fluoropyridin-2-yl)(phenyl)methyl]- 4-fluoro-1-[2-(5-methoxy-1- methyl-1H-1,2,4-triazol-3- yl)acetyl]pyrrolidine-2- carboxamide |
| GZ-744 | 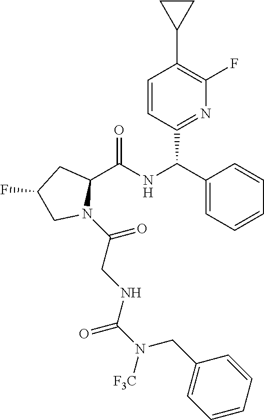 [SMILES: O=C(N[C@@H](c1ccccc1)c1ccc(C2CC2)c(F)n1)[C@@H]1C[C@@H](F)CN1C(=O)CNC(=O)N(Cc1ccccc1)C(F)(F)F] | (2S,4R)-1-(2- {[benzyl(trifluoromethyl)carbamoyl] amino} acetyl)-N-[(S)-(5- cyclopropyl-6-fluoropyridin-2- yl)(phenyl)methyl]-4- fluoropyrrolidine-2-carboxamide |
| GZ-745 | 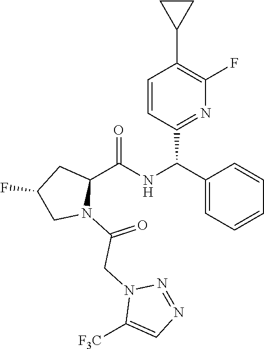 [SMILES: O=C(N[C@@H](c1ccccc1)c1ccc(C2CC2)c(F)n1)[C@@H]1C[C@@H](F)CN1C(=O)Cn1nncc1C(F)(F)F] | (2S,4R)-N-[(S)-(5-cyclopropyl-6- fluoropyridin-2-yl)(phenyl)methyl]- 4-fluoro-1-{2-[5-(trifluoromethyl)- 1H-1,2,3-triazol-1- yl]acetyl} pyrrolidine-2- carboxamide |
| GZ-746 | 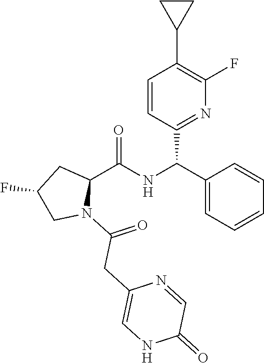 [SMILES: O=C(N[C@@H](c1ccccc1)c1ccc(C2CC2)c(F)n1)[C@@H]1C[C@@H](F)CN1C(=O)Cc1c[nH]c(=O)cn1] | (2S,4R)-N-[(S)-(5-cyclopropyl-6- fluoropyridin-2-yl)(phenyl)methyl]- 4-fluoro-1-[2-(5-oxo-4,5- dihydropyrazin-2- yl)acetyl]pyrrolidine-2- carboxamide |
| GZ-747 | 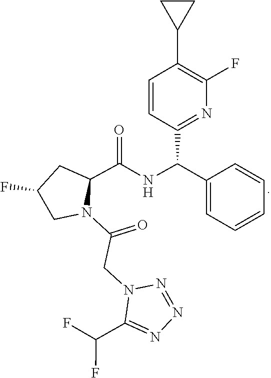 [SMILES: O=C(N[C@@H](c1ccccc1)c1ccc(C2CC2)c(F)n1)[C@@H]1C[C@@H](F)CN1C(=O)Cn1nnnc1C(F)F] | (2S,4R)-N-[(S)-(5-cyclopropyl-6- fluoropyridin-2-yl)(phenyl)methyl]- 1-{2-[5-(difluoromethyl)-1H-1,2,3- triazol-1-yl]acetyl}-4- fluoropyrrolidine-2-carboxamide |
| GZ-748 | 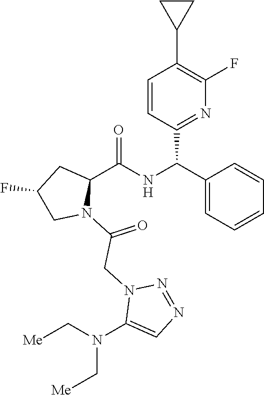 [SMILES: CCN(CC)c1cnnn1CC(=O)N1C[C@H](F)C[C@H]1C(=O)N[C@@H](c1ccccc1)c1ccc(C2CC2)c(F)n1] | (2S,4R)-N-[(S)-(5-cyclopropyl-6- fluoropyridin-2-yl)(phenyl)methyl]- 1-{2-[5-(diethylamino)-1H-1,2,3- triazol-1-yl]acetyl}-4- fluoropyrrolidine-2-carboxamide |
| GZ-749 | 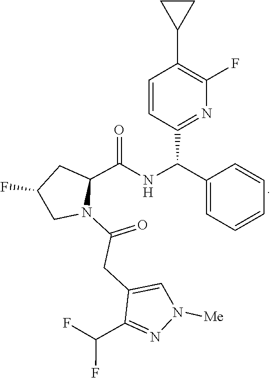 [SMILES: Cn1cc(CC(=O)N2C[C@H](F)C[C@H]2C(=O)N[C@@H](c2ccccc2)c2ccc(C3CC3)c(F)n2)c(C(F)F)n1] | (2S,4R)-N-[(S)-(5-cyclopropyl-6- fluoropyridin-2-yl)(phenyl)methyl]- 1-{2-[3-(difluoromethyl)-1-methyl- 1H-pyrazol-4-yl]acetyl }-4- fluoropyrrolidine-2-carboxamide |
| GZ-750 | 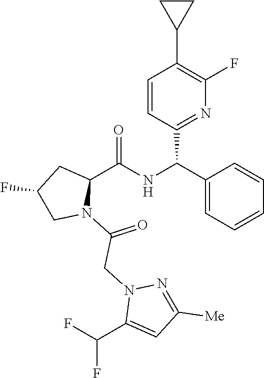 [SMILES: Cc1cc(C(F)F)n(CC(=O)N2C[C@H](F)C[C@H]2C(=O)N[C@@H](c2ccccc2)c2ccc(C3CC3)c(F)n2)n1] | (2S,4R)-N-[(S)-(5-cyclopropyl-6- fluoropyridin-2-yl)(phenyl)methyl]- 1-{2-[5-(difluoromethyl)-3-methyl- 1H-pyrazol-1-yl]acetyl}-4- fluoropyrrolidine-2-carboxamide |
| GZ-751 | 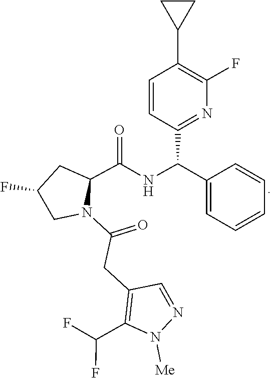 [SMILES: Cn1ncc(CC(=O)N2C[C@H](F)C[C@H]2C(=O)N[C@@H](c2ccccc2)c2ccc(C3CC3)c(F)n2)c1C(F)F] | (2S,4R)-N-[(S)-(5-cyclopropyl-6- fluoropyridin-2-yl)(phenyl)methyl]- 1-{2-[5-(difluoromethyl)-1-methyl- 1H-pyrazol-4-yl]acetyl}-4- fluoropyrrolidine-2-carboxamide |
| GZ-752 | 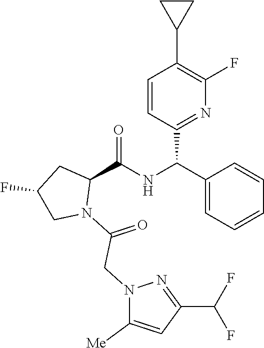 [SMILES: Cc1cc(C(F)F)nn1CC(=O)N1C[C@H](F)C[C@H]1C(=O)N[C@@H](c1ccccc1)c1ccc(C2CC2)c(F)n1] | (2S,4R)-N-[(S)-(5-cyclopropyl-6- fluoropyridin-2-yl)(phenyl)methyl]- 1-{2-[3-(difluoromethyl)-5-methyl- 1H-pyrazol-1-yl]acetyl }-4- fluoropyrrolidine-2-carboxamide |
| GZ-753 | 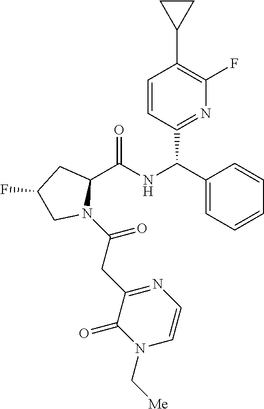 [SMILES: CCn1ccnc(CC(=O)N2C[C@H](F)C[C@H]2C(=O)N[C@@H](c2ccccc2)c2ccc(C3CC3)c(F)n2)c1=O] | (2S,4R)-N-[(S)-(5-cyclopropyl-6- fluoropyridin-2-yl)(phenyl)methyl]- 1-[2-(4-ethyl-3-oxo-3,4- dihydropyrazin-2-yl)acetyl]-4- fluoropyrrolidine-2-carboxamide |
| GZ-754 | 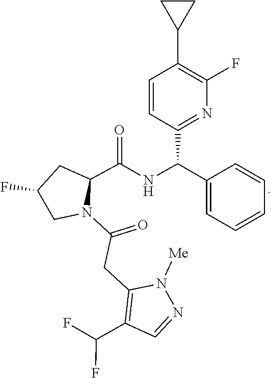 [SMILES: Cn1ncc(C(F)F)c1CC(=O)N1C[C@H](F)C[C@H]1C(=O)N[C@@H](c1ccccc1)c1ccc(C2CC2)c(F)n1] | (2S,4R)-N-[(S)-(5-cyclopropyl-6- fluoropyridin-2-yl)(phenyl)methyl]- 1-{2-[4-(difluoromethyl)-1-methyl- 1H-pyrazol-5-yl]acetyl}-4- fluoropyrrolidine-2-carboxamide |
| GZ-755 | 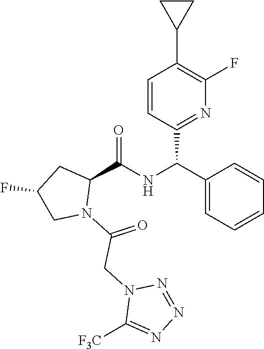 [SMILES: O=C(N[C@@H](c1ccccc1)c1ccc(C2CC2)c(F)n1)[C@@H]1C[C@@H](F)CN1C(=O)Cn1nnnc1C(F)(F)F] | (2S,4R)-N-[(S)-(5-cyclopropyl-6- fluoropyridin-2-yl)(phenyl)methyl]- 4-fluoro-1-{2-[5-(trifluoromethyl)- 1H-1,2,3,4-tetrazol-1- yl]acetyl} pyrrolidine-2- carboxamide |
| GZ-756 | 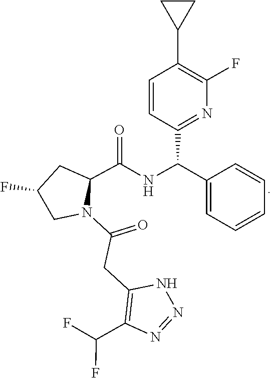 [SMILES: O=C(N[C@@H](c1ccccc1)c1ccc(C2CC2)c(F)n1)[C@@H]1C[C@@H](F)CN1C(=O)Cc1[nH]nnc1C(F)F] | (2S,4R)-N-[(S)-(5-cyclopropyl-6- fluoropyridin-2-yl)(phenyl)methyl]- 1-{2-[4-(difluoromethyl)-1H-1,2,3- triazol-5-yl]acetyl}-4- fluoropyrrolidine-2-carboxamide |
| GZ-757 | 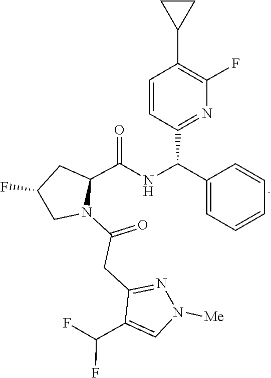 [SMILES: Cn1cc(C(F)F)c(CC(=O)N2C[C@H](F)C[C@H]2C(=O)N[C@@H](c2ccccc2)c2ccc(C3CC3)c(F)n2)n1] | (2S,4R)-N-[(S)-(5-cyclopropyl-6- fluoropyridin-2-yl)(phenyl)methyl]- 1-{2-[4-(difluoromethyl)-1-methyl- 1H-pyrazol-3-yl]acetyl}-4- fluoropyrrolidine-2-carboxamide |
| GZ-758 | 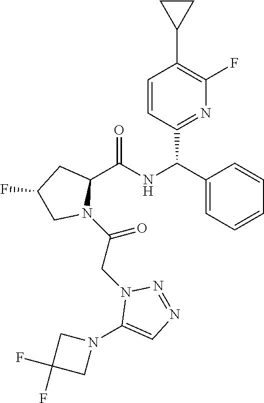 [SMILES: O=C(N[C@@H](c1ccccc1)c1ccc(C2CC2)c(F)n1)[C@@H]1C[C@@H](F)CN1C(=O)Cn1nncc1N1CC(F)(F)C1] | (2S,4R)-N-[(S)-(5-cyclopropyl-6- fluoropyridin-2-yl)(phenyl)methyl]- 1-{2-[5-(3,3-difluoroazetidin-1-yl)- 1H-1,2,3-triazol-1-yl]acetyl}-4- fluoropyrrolidine-2-carboxamide |
| GZ-759 | 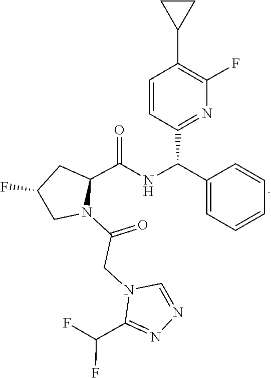 [SMILES: O=C(N[C@@H](c1ccccc1)c1ccc(C2CC2)c(F)n1)[C@@H]1C[C@@H](F)CN1C(=O)Cn1cnnc1C(F)F] | (2S,4R)-N-[(S)-(5-cyclopropyl-6- fluoropyridin-2-yl)(phenyl)methyl]- 1-{2-[3-(difluoromethyl)-4H-1,2,4- triazol-4-yl]acetyl}-4- fluoropyrrolidine-2-carboxamide |
| GZ-760 | 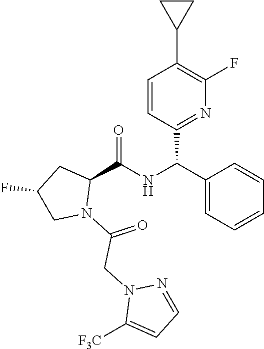 [SMILES: O=C(N[C@@H](c1ccccc1)c1ccc(C2CC2)c(F)n1)[C@@H]1C[C@@H](F)CN1C(=O)Cn1nccc1C(F)(F)F] | (2S,4R)-N-[(S)-(5-cyclopropyl-6- fluoropyridin-2-yl)(phenyl)methyl]- 4-fluoro-1-{2-[5-(trifluoromethyl)- 1H-pyrazol-1-yl]acetyl} pyrrolidine- 2-carboxamide |
| GZ-761 | 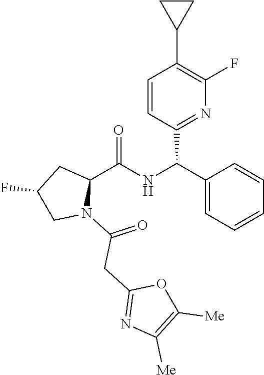 [SMILES: Cc1nc(CC(=O)N2C[C@H](F)C[C@H]2C(=O)N[C@@H](c2ccccc2)c2ccc(C3CC3)c(F)n2)oc1C] | (2S,4R)-N-[(S)-(5-cyclopropyl-6- fluoropyridin-2-yl)(phenyl)methyl]- 1-[2-(4,5-dimethyl-1,3-oxazol-2- yl)acetyl]-4-fluoropyrrolidine-2- carboxamide |
| GZ-762 | 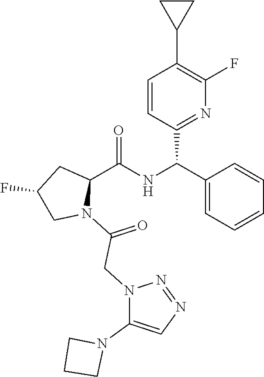 [SMILES: O=C(N[C@@H](c1ccccc1)c1ccc(C2CC2)c(F)n1)[C@@H]1C[C@@H](F)CN1C(=O)Cn1nncc1N1CCC1] | (2S,4R)-1-{2-[5-(azetidin-1-yl)-1H- 1,2,3-triazol-1-yl]acetyl}-N-[(S)-(5- cyclopropyl-6-fluoropyridin-2- yl)(phenyl)methyl]-4- fluoropyrrolidine-2-carboxamide |
| GZ-763 | 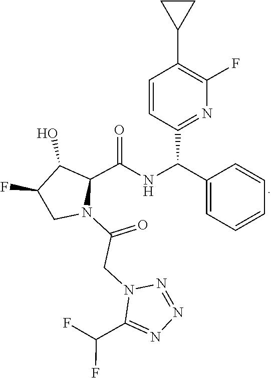 [SMILES: O=C(N[C@@H](c1ccccc1)c1ccc(C2CC2)c(F)n1)[C@@H]1[C@@H](O)[C@H](F)CN1C(=O)Cn1nnnc1C(F)F] | (2S,3R,4R)-N-[(S)-(5-cyclopropyl- 6-fluoropyridin-2- yl)(phenyl)methyl]-1-{2-[5- (difluoromethyl)-1H-1,2,3,4- tetrazol-1-yl]acetyl}-4-fluoro-3- hydroxypyrrolidine-2-carboxamide |
| GZ-764 | 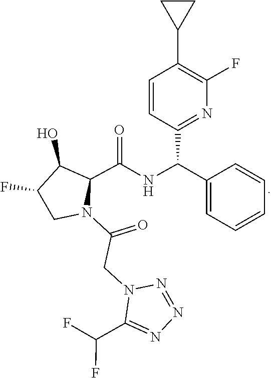 [SMILES: O=C(N[C@@H](c1ccccc1)c1ccc(C2CC2)c(F)n1)[C@@H]1[C@H](O)[C@@H](F)CN1C(=O)Cn1nnnc1C(F)F] | (2S,3S,4S)-N-[(S)-(5-cyclopropyl- 6-fluoropyridin-2- yl)(phenyl)methyl]-1-{2-[5- (difluoromethyl)-1H-1,2,3,4- tetrazol-1-yl]acetyl}-4-fluoro-3- hydroxypyrrolidine-2-carboxamide |
| GZ-765 | 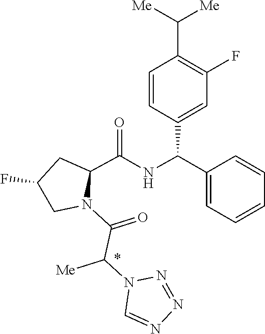 [SMILES: CC(C)c1ccc([C@@H](NC(=O)[C@@H]2C[C@@H](F)CN2C(=O)C(C)n2cnnn2)c2ccccc2)cc1F] | (2S,4R)-1-[(2S)-3-carbamoyl-2- acetamidopropanoyl]-4-fluoro-N- [(S)-[3-fluoro-4-(propan-2- yl)phenyl](phenyl)methyl]pyrrolidine- 2-carboxamide |
| GZ-766 | 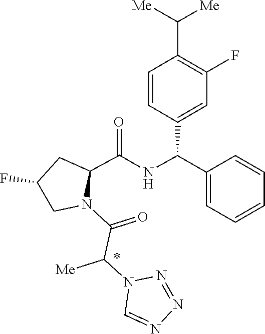 [SMILES: CC(C)c1ccc([C@@H](NC(=O)[C@@H]2C[C@@H](F)CN2C(=O)C(C)n2cnnn2)c2ccccc2)cc1F] | (2S,4R)-4-fluoro-N-[(S)-[3-fluoro- 4-(propan-2- yl)phenyl](phenyl)methyl]-1-[(2R) or (2S)-2-(1H-1,2,3,4-tetrazol-1- yl)propanoyl]pyrrolidine-2- carboxamide |
| GZ-767 | 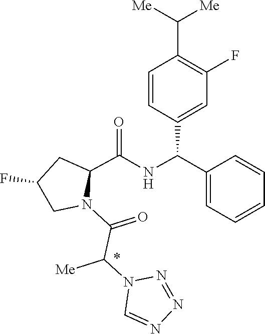 [SMILES: CC(C)c1ccc([C@@H](NC(=O)[C@@H]2C[C@@H](F)CN2C(=O)C(C)n2cnnn2)c2ccccc2)cc1F] | (2S,4R)-4-fluoro-N-[(S)-[3-fluoro- 4-(propan-2- yl)phenyl](phenyl)methyl]-1-[(2S) or (2R)-2-(1H-1,2,3,4-tetrazol-1- yl)propanoyl]pyrrolidine-2- carboxamide |
| GZ-768 | 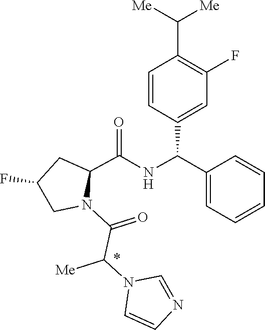 [SMILES: CC(C)c1ccc([C@@H](NC(=O)[C@@H]2C[C@@H](F)CN2C(=O)C(C)n2ccnc2)c2ccccc2)cc1F] | (2S,4R)-4-fluoro-N-[(S)-[3-fluoro- 4-(propan-2- yl)phenyl](phenyl)methyl]-1-[(2R) or (2S)-2-(1H-imidazol-1- yl)propanoyl]pyrrolidine-2- carboxamide |
| GZ-769 | 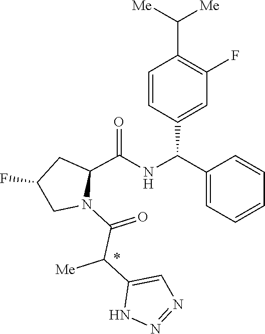 [SMILES: CC(C)c1ccc([C@@H](NC(=O)[C@@H]2C[C@@H](F)CN2C(=O)C(C)c2cnn[nH]2)c2ccccc2)cc1F] | (2S,4R)-4-fluoro-N-[(S)-[3-fluoro- 4-(propan-2- yl)phenyl](phenyl)methyl]-1-[(2R) or (2S)-2-(1H-1,2,3-triazol-5- yl)propanoyl]pyrrolidine-2- carboxamide |
| GZ-770 | 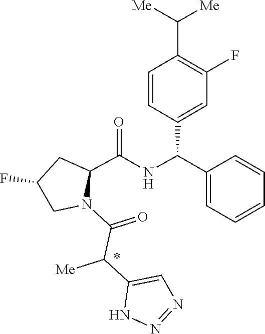 [SMILES: CC(C)c1ccc([C@@H](NC(=O)[C@@H]2C[C@@H](F)CN2C(=O)C(C)c2cnn[nH]2)c2ccccc2)cc1F] | (2S,4R)-4-fluoro-N-[(S)-[3-fluoro- 4-(propan-2- yl)phenyl](phenyl)methyl]-1-[(2S) or (2R)-2-(1H-1,2,3-triazol-5- yl)propanoyl]pyrrolidine-2- carboxamide |
| GZ-771 | 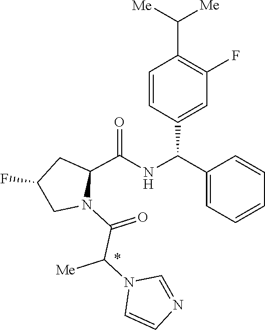 [SMILES: CC(C)c1ccc([C@@H](NC(=O)[C@@H]2C[C@@H](F)CN2C(=O)C(C)n2ccnc2)c2ccccc2)cc1F] | (2S,4R)-4-fluoro-N-[(S)-[3-fluoro- 4-(propan-2- yl)phenyl](phenyl)methyl]-1-[(2S) or (2R)-2-(1H-imidazol-1- yl)propanoyl]pyrrolidine-2- carboxamide |
| GZ-772 | 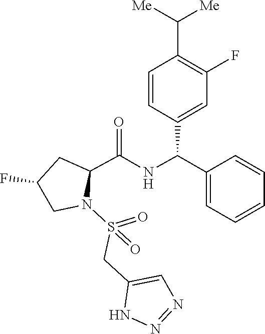 [SMILES: CC(C)c1ccc([C@@H](NC(=O)[C@@H]2C[C@@H](F)CN2S(=O)(=O)Cc2cnn[nH]2)c2ccccc2)cc1F] | (2S,4R)-4-fluoro-N-[(S)-[3-fluoro- 4-(propan-2- yl)phenyl](phenyl)methyl]-1-[(1H- 1,2,3-triazol-5- yl)methanesulfonyl]pyrrolidine-2- carboxamide |
| GZ-773 | 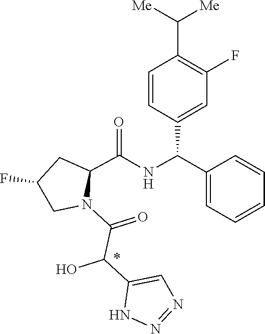 [SMILES: CC(C)c1ccc([C@@H](NC(=O)[C@@H]2C[C@@H](F)CN2C(=O)C(O)c2cnn[nH]2)c2ccccc2)cc1F] | (2S,4R)-4-fluoro-N-[(S)-[3-fluoro- 4-(propan-2- yl)phenyl](phenyl)methyl]-1-[(2R) or (2S)-2-hydroxy-2-(1H-1,2,3- triazol-5-yl)acetyl]pyrrolidine-2- carboxamide |
| GZ-774 | 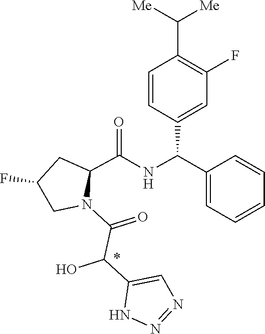 [SMILES: CC(C)c1ccc([C@@H](NC(=O)[C@@H]2C[C@@H](F)CN2C(=O)C(O)c2cnn[nH]2)c2ccccc2)cc1F] | (2S,4R)-4-fluoro-N-[(S)-[3-fluoro- 4-(propan-2- yl)phenyl](phenyl)methyl]-1-[(2S) or (2R)-2-hydroxy-2-(1H-1,2,3- triazol-5-yl)acetyl]pyrrolidine-2- carboxamide |
| GZ-775 | 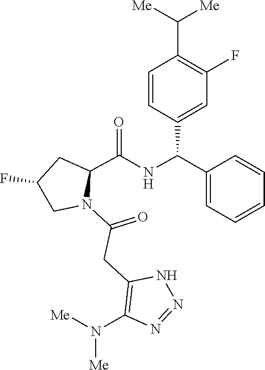 [SMILES: CC(C)c1ccc([C@@H](NC(=O)[C@@H]2C[C@@H](F)CN2C(=O)Cc2[nH]nnc2N(C)C)c2ccccc2)cc1F] | (2S,4R)-1-{2-[4-(dimethylamino)- 1H-1,2,3-triazol-5-yl]acetyl}-4- fluoro-N-[(S)-[3-fluoro-4-(propan- 2- yl)phenyl](phenyl)methyl]pyrrolidine- 2-carboxamide |
| GZ-776 | 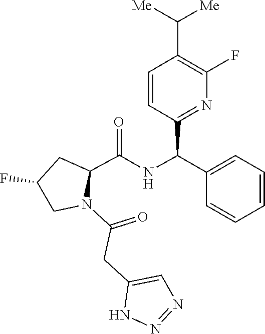 [SMILES: CC(C)c1ccc([C@H](NC(=O)[C@@H]2C[C@@H](F)CN2C(=O)Cc2cnn[nH]2)c2ccccc2)nc1F] | (2S,4R)-4-fluoro-N-[(R*)-[6- fluoro-5-(propan-2-yl)pyridin-2- yl](phenyl)methyl]-1-[2-(1H-1,2,3- triazol-5-yl)acetyl]pyrrolidine-2- carboxamide |
| GZ-777 | 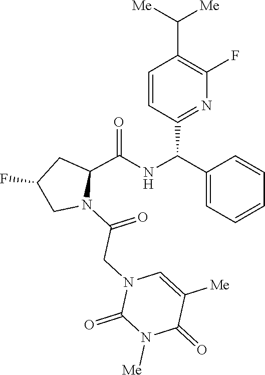 [SMILES: Cc1cn(CC(=O)N2C[C@H](F)C[C@H]2C(=O)N[C@@H](c2ccccc2)c2ccc(C(C)C)c(F)n2)c(=O)n(C)c1=O] | (2S,4R)-1-[2-(3,5-dimethyl-2,4- dioxo-1,2,3,4-tetrahydropyrimidin- 1-yl)acetyl]-4-fluoro-N-[(R*)-[6- fluoro-5-(propan-2-yl)pyridin-2- yl](phenyl)methyl]pyrrolidine-2- carboxamide |
| GZ-778 | 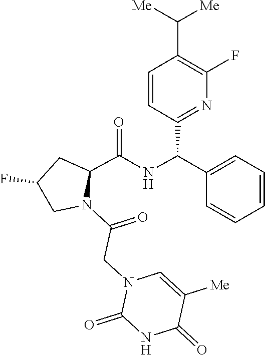 [SMILES: Cc1cn(CC(=O)N2C[C@H](F)C[C@H]2C(=O)N[C@@H](c2ccccc2)c2ccc(C(C)C)c(F)n2)c(=O)[nH]c1=O] | (2S,4R)-4-fluoro-N-[(R*)-[6- fluoro-5-(propan-2-yl)pyridin-2- yl](phenyl)methyl]-1-[2-(5-methyl- 2,4-dioxo-1,2,3,4- tetrahydropyrimidin-1- yl)acetyl]pyrrolidine-2- carboxamide |
| GZ-779 | 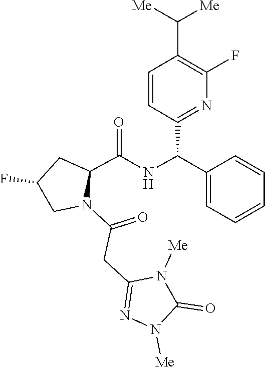 [SMILES: CC(C)c1ccc([C@@H](NC(=O)[C@@H]2C[C@@H](F)CN2C(=O)Cc2nn(C)c(=O)n2C)c2ccccc2)nc1F] | (2S,4R)-1-[2-(1,4-dimethyl-5-oxo- 4,5-dihydro-1H-1,2,4-triazol-3- yl)acetyl]-4-fluoro-N-[(S)-[6- fluoro-5-(propan-2-yl)pyridin-2- yl](phenyl)methyl]pyrrolidine-2- carboxamide |
| GZ-780 | 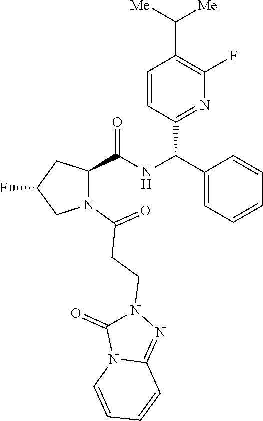 [SMILES: CC(C)c1ccc([C@@H](NC(=O)[C@@H]2C[C@@H](F)CN2C(=O)CCn2nc3ccccn3c2=O)c2ccccc2)nc1F] | (2S,4R)-4-fluoro-N-[(S)-[6-fluoro- 5-(propan-2-yl)pyridin-2- yl](phenyl)methyl]-1-(3-{3-oxo- 2H,3H-[1,2,4]triazolo[4,3- alpyridin-2- yl} propanoyl)pyrrolidine-2- carboxamide |
| GZ-781 | 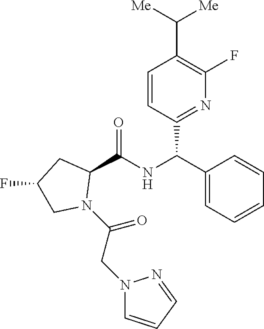 [SMILES: CC(C)c1ccc([C@@H](NC(=O)[C@@H]2C[C@@H](F)CN2C(=O)Cn2cccn2)c2ccccc2)nc1F] | (2S,4R)-4-fluoro-N-[(S)-[6-fluoro- 5-(propan-2-yl)pyridin-2- yl](phenyl)methyl]-1-[2-(1H- pyrazol-1-yl)acetyl]pyrrolidine-2- carboxamide |
| GZ-782 | 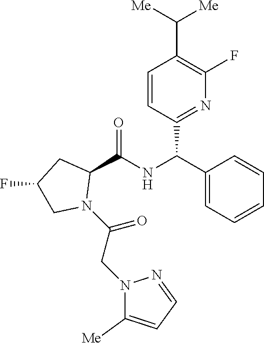 [SMILES: Cc1ccnn1CC(=O)N1C[C@H](F)C[C@H]1C(=O)N[C@@H](c1ccccc1)c1ccc(C(C)C)c(F)n1] | (2S,4R)-4-fluoro-N-[(S)-[6-fluoro- 5-(propan-2-yl)pyridin-2- yl](phenyl)methyl]-1-[2-(5-methyl- 1H-pyrazol-1-yl)acetyl]pyrrolidine- 2-carboxamide |
| GZ-783 | 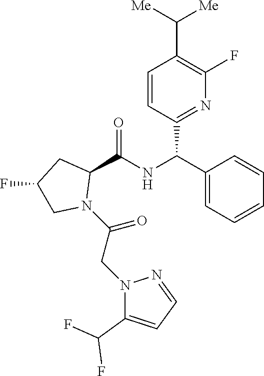 [SMILES: CC(C)c1ccc([C@@H](NC(=O)[C@@H]2C[C@@H](F)CN2C(=O)Cn2nccc2C(F)F)c2ccccc2)nc1F] | (2S,4R)-1-{2-[5-(difluoromethyl)- 1H-pyrazol-1-yl]acetyl}-4-fluoro- N-[(S)-[6-fluoro-5-(propan-2- yl)pyridin-2- yl](phenyl)methyl]pyrrolidine-2- carboxamide |
| GZ-784 | 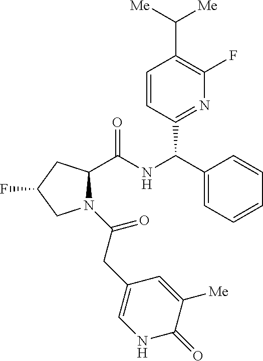 [SMILES: Cc1cc(CC(=O)N2C[C@H](F)C[C@H]2C(=O)N[C@@H](c2ccccc2)c2ccc(C(C)C)c(F)n2)c[nH]c1=O] | (2S,4R)-4-fluoro-N-[(S)-[6-fluoro- 5-(propan-2-yl)pyridin-2- yl](phenyl)methyl]-1-[2-(5-methyl- 6-oxo-1,6-dihydropyridin-3- yl)acetyl]pyrrolidine-2- carboxamide |
| GZ-785 | 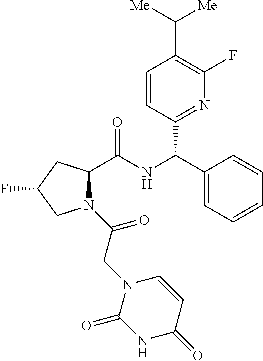 [SMILES: CC(C)c1ccc([C@@H](NC(=O)[C@@H]2C[C@@H](F)CN2C(=O)Cn2ccc(=O)[nH]c2=O)c2ccccc2)nc1F] | (2S,4R)-1-[2-(2,4-dioxo-1,2,3,4- tetrahydropyrimidin-1-yl)acetyl]-4- fluoro-N-[(S)-[6-fluoro-5-(propan- 2-yl)pyridin-2- yl](phenyl)methyl]pyrrolidine-2- carboxamide |
| GZ-786 | 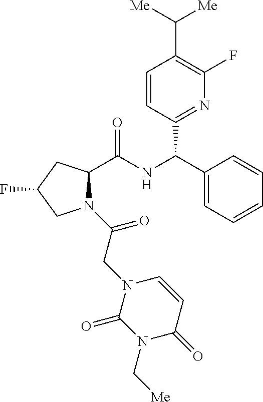 [SMILES: CCn1c(=O)ccn(CC(=O)N2C[C@H](F)C[C@H]2C(=O)N[C@@H](c2ccccc2)c2ccc(C(C)C)c(F)n2)c1=O] | (2S,4R)-1-[2-(3-ethyl-2,4-dioxo- 1,2,3,4-tetrahydropyrimidin-1- yl)acetyl]-4-fluoro-N-[(S)-[6- fluoro-5-(propan-2-yl)pyridin-2- yl](phenyl)methyl]pyrrolidine-2- carboxamide |
| GZ-787 | 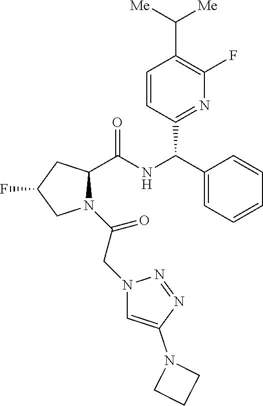 [SMILES: CC(C)c1ccc([C@@H](NC(=O)[C@@H]2C[C@@H](F)CN2C(=O)Cn2cc(N3CCC3)nn2)c2ccccc2)nc1F] | (2S,4R)-1-{2-[4-(azetidin-1-yl)-1H- 1,2,3-triazol-1-yl]acetyl}-4-fluoro- N-[(S)-[6-fluoro-5-(propan-2- yl)pyridin-2- yl](phenyl)methyl]pyrrolidine-2- carboxamide |
| GZ-788 | 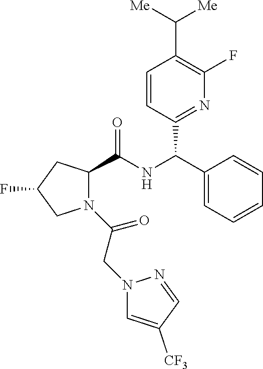 [SMILES: CC(C)c1ccc([C@@H](NC(=O)[C@@H]2C[C@@H](F)CN2C(=O)Cn2cc(C(F)(F)F)cn2)c2ccccc2)nc1F] | (2S,4R)-4-fluoro-N-[(S)-[6-fluoro- 5-(propan-2-yl)pyridin-2- yl](phenyl)methyl]-1-{2-[4- (trifluoromethyl)-1H-pyrazol-1- yl]acetyl} pyrrolidine-2- carboxamide |
| GZ-789 | 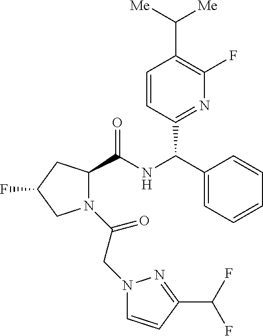 [SMILES: CC(C)c1ccc([C@@H](NC(=O)[C@@H]2C[C@@H](F)CN2C(=O)Cn2ccc(C(F)F)n2)c2ccccc2)nc1F] | (2S,4R)-1-{2-[3-(difluoromethyl)- 1H-pyrazol-1-yl]acetyl}-4-fluoro- N-[(S)-[6-fluoro-5-(propan-2- yl)pyridin-2- yl](phenyl)methyl]pyrrolidine-2- carboxamide |
| GZ-790 | 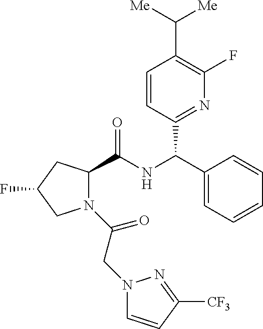 [SMILES: CC(C)c1ccc([C@@H](NC(=O)[C@@H]2C[C@@H](F)CN2C(=O)Cn2ccc(C(F)(F)F)n2)c2ccccc2)nc1F] | (2S,4R)-4-fluoro-N-[(S)-[6-fluoro- 5-(propan-2-yl)pyridin-2- yl](phenyl)methyl]-1-{2-[3- (trifluoromethyl)-1H-pyrazol-1- yl]acetyl} pyrrolidine-2- carboxamide |
| GZ-791 | 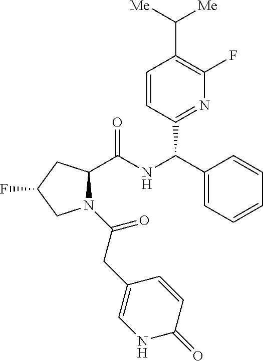 [SMILES: CC(C)c1ccc([C@@H](NC(=O)[C@@H]2C[C@@H](F)CN2C(=O)Cc2ccc(=O)[nH]c2)c2ccccc2)nc1F] | (2S,4R)-4-fluoro-N-[(S)-[6-fluoro- 5-(propan-2-yl)pyridin-2- yl](phenyl)methyl]-1-[2-(6-oxo- 1,6-dihydropyridin-3- yl)acetyl]pyrrolidine-2- carboxamide |
| GZ-792 | 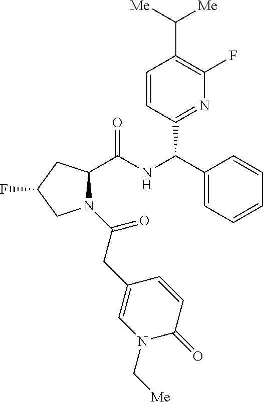 [SMILES: CCn1cc(CC(=O)N2C[C@H](F)C[C@H]2C(=O)N[C@@H](c2ccccc2)c2ccc(C(C)C)c(F)n2)ccc1=O] | (2S,4R)-1-[2-(1-ethyl-6-oxo-1,6- dihydropyridin-3-yl)acetyl]-4- fluoro-N-[(S)-[6-fluoro-5-(propan- 2-yl)pyridin-2- yl](phenyl)methyl]pyrrolidine-2- carboxamide |
| GZ-793 | 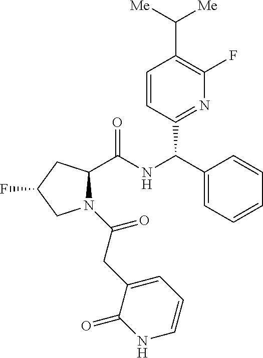 [SMILES: CC(C)c1ccc([C@@H](NC(=O)[C@@H]2C[C@@H](F)CN2C(=O)Cc2ccc[nH]c2=O)c2ccccc2)nc1F] | (2S,4R)-4-fluoro-N-[(S)-[6-fluoro- 5-(propan-2-yl)pyridin-2- yl](phenyl)methyl]-1-[2-(2-oxo- 1,2-dihydropyridin-3- yl)acetyl]pyrrolidine-2- carboxamide |
| GZ-794 | 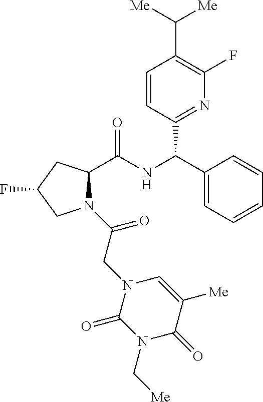 [SMILES: CCn1c(=O)c(C)cn(CC(=O)N2C[C@H](F)C[C@H]2C(=O)N[C@@H](c2ccccc2)c2ccc(C(C)C)c(F)n2)c1=O] | (2S,4R)-1-[2-(3-ethyl-5-methyl- 2,4-dioxo-1,2,3,4- tetrahydropyrimidin-1-yl)acetyl]-4- fluoro-N-[(S)-[6-fluoro-5-(propan- 2-yl)pyridin-2- yl](phenyl)methyl]pyrrolidine-2- carboxamide |
| GZ-795 | 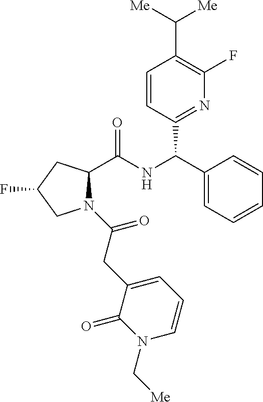 [SMILES: CCn1cccc(CC(=O)N2C[C@H](F)C[C@H]2C(=O)N[C@@H](c2ccccc2)c2ccc(C(C)C)c(F)n2)c1=O] | (2S,4R)-1-[2-(1-ethyl-2-oxo-1,2- dihydropyridin-3-yl)acetyl]-4- fluoro-N-[(S)-[6-fluoro-5-(propan- 2-yl)pyridin-2- yl](phenyl)methyl]pyrrolidine-2- carboxamide |
| GZ-796 | 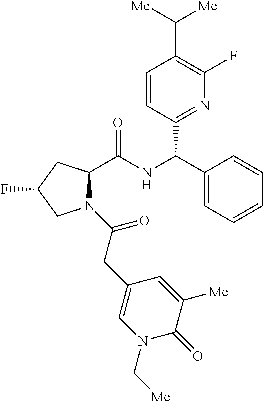 [SMILES: CCn1cc(CC(=O)N2C[C@H](F)C[C@H]2C(=O)N[C@@H](c2ccccc2)c2ccc(C(C)C)c(F)n2)cc(C)c1=O] | (2S,4R)-1-[2-(1-ethyl-5-methyl-6- oxo-1,6-dihydropyridin-3- yl)acetyl]-4-fluoro-N-[(S)-[6- fluoro-5-(propan-2-yl)pyridin-2- yl](phenyl)methyl]pyrrolidine-2- carboxamide |
| GZ-797 | 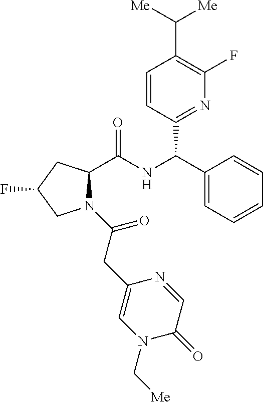 [SMILES: CCn1cc(CC(=O)N2C[C@H](F)C[C@H]2C(=O)N[C@@H](c2ccccc2)c2ccc(C(C)C)c(F)n2)ncc1=O] | (2S,4R)-1-[2-(4-ethyl-5-oxo-4,5- dihydropyrazin-2-yl)acetyl]-4- fluoro-N-[(S)-[6-fluoro-5-(propan- 2-yl)pyridin-2- yl](phenyl)methyl]pyrrolidine-2- carboxamide |
| GZ-798 | 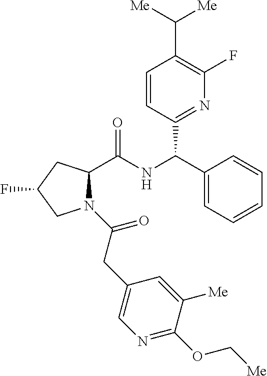 [SMILES: CCOc1ncc(CC(=O)N2C[C@H](F)C[C@H]2C(=O)N[C@@H](c2ccccc2)c2ccc(C(C)C)c(F)n2)cc1C] | (2S,4R)-1-[2-(6-ethoxy-5- methylpyridin-3-yl)acetyl]-4- fluoro-N-[(S)-[6-fluoro-5-(propan- 2-yl)pyridin-2- yl](phenyl)methyl]pyrrolidine-2- carboxamide |
| GZ-799 | 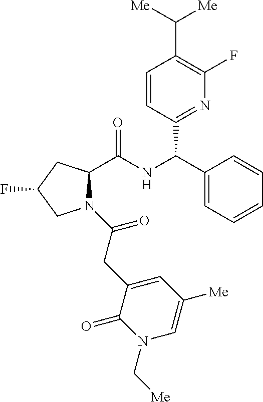 [SMILES: CCn1cc(C)cc(CC(=O)N2C[C@H](F)C[C@H]2C(=O)N[C@@H](c2ccccc2)c2ccc(C(C)C)c(F)n2)c1=O] | (2S,4R)-1-[2-(1-ethyl-5-methyl-2- oxo-1,2-dihydropyridin-3- yl)acetyl]-4-fluoro-N-[(S)-[6- fluoro-5-(propan-2-yl)pyridin-2- yl](phenyl)methyl]pyrrolidine-2- carboxamide |
| GZ-800 | 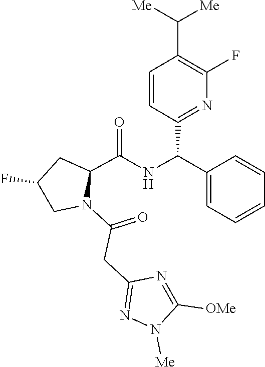 [SMILES: COc1nc(CC(=O)N2C[C@H](F)C[C@H]2C(=O)N[C@@H](c2ccccc2)c2ccc(C(C)C)c(F)n2)nn1C] | (2S,4R)-4-fluoro-N-[(S)-[6-fluoro- 5-(propan-2-yl)pyridin-2- yl](phenyl)methyl]-1-[2-(5- methoxy-1-methyl-1H-1,2,4- triazol-3-yl)acetyl]pyrrolidine-2- carboxamide |
| GZ-801 | 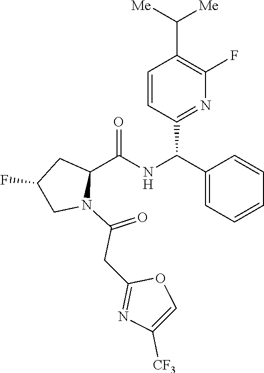 [SMILES: CC(C)c1ccc([C@@H](NC(=O)[C@@H]2C[C@@H](F)CN2C(=O)Cc2nc(C(F)(F)F)co2)c2ccccc2)nc1F] | (2S,4R)-4-fluoro-N-[(S)-[6-fluoro- 5-(propan-2-yl)pyridin-2- yl](phenyl)methyl]-1-{2-[4- (trifluoromethyl)-1,3-oxazol-2- yl]acetyl} pyrrolidine-2- carboxamide |
| GZ-802 | 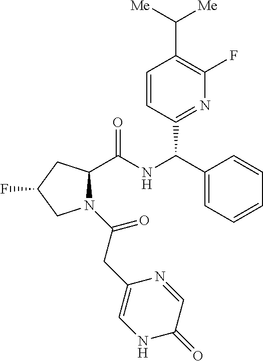 [SMILES: CC(C)c1ccc([C@@H](NC(=O)[C@@H]2C[C@@H](F)CN2C(=O)Cc2c[nH]c(=O)cn2)c2ccccc2)nc1F] | (2S,4R)-4-fluoro-N-[(S)-[6-fluoro- 5-(propan-2-yl)pyridin-2- yl](phenyl)methyl]-1-[2-(5-oxo- 4,5-dihydropyrazin-2- yl)acetyl]pyrrolidine-2- carboxamide |
| GZ-803 | 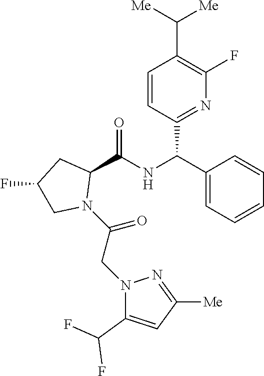 [SMILES: Cc1cc(C(F)F)n(CC(=O)N2C[C@H](F)C[C@H]2C(=O)N[C@@H](c2ccccc2)c2ccc(C(C)C)c(F)n2)n1] | (2S,4R)-1-{2-[5-(difluoromethyl)- 3-methyl-1H-pyrazol-1-yl]acetyl }- 4-fluoro-N-[(S)-[6-fluoro-5- (propan-2-yl)pyridin-2- yl](phenyl)methyl]pyrrolidine-2- carboxamide |
| GZ-804 | 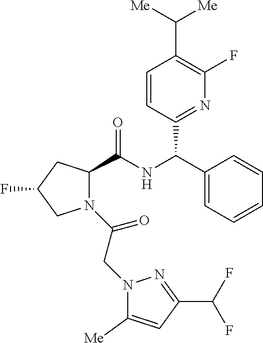 [SMILES: Cc1cc(C(F)F)nn1CC(=O)N1C[C@H](F)C[C@H]1C(=O)N[C@@H](c1ccccc1)c1ccc(C(C)C)c(F)n1] | (2S,4R)-1-{2-[3-(difluoromethyl)- 5-methyl-1H-pyrazol-1-yl]acetyl}- 4-fluoro-N-[(S)-[6-fluoro-5- (propan-2-yl)pyridin-2- yl](phenyl)methyl]pyrrolidine-2- carboxamide |
| GZ-805 | 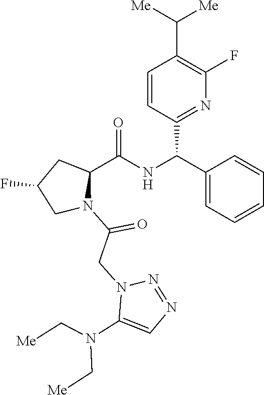 [SMILES: CCN(CC)c1cnnn1CC(=O)N1C[C@H](F)C[C@H]1C(=O)N[C@@H](c1ccccc1)c1ccc(C(C)C)c(F)n1] | (2S,4R)-1-{2-[5-(diethylamino)- 1H-1,2,3-triazol-1-yl]acetyl}-4- fluoro-N-[(S)-[6-fluoro-5-(propan- 2-yl)pyridin-2- yl](phenyl)methyl]pyrrolidine-2- carboxamide |
| GZ-806 | 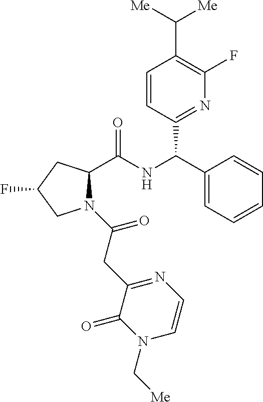 [SMILES: CCn1ccnc(CC(=O)N2C[C@H](F)C[C@H]2C(=O)N[C@@H](c2ccccc2)c2ccc(C(C)C)c(F)n2)c1=O] | (2S,4R)-1-[2-(4-ethyl-3-oxo-3,4- dihydropyrazin-2-yl)acetyl]-4- fluoro-N-[(S)-[6-fluoro-5-(propan- 2-yl)pyridin-2- yl](phenyl)methyl]pyrrolidine-2- carboxamide |
| GZ-807 | 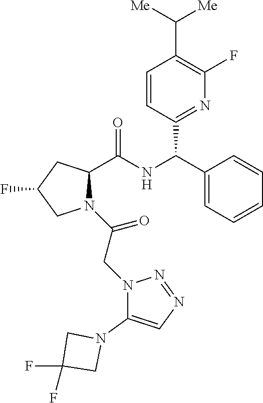 [SMILES: CC(C)c1ccc([C@@H](NC(=O)[C@@H]2C[C@@H](F)CN2C(=O)Cn2nncc2N2CC(F)(F)C2)c2ccccc2)nc1F] | (2S,4R)-1-{2-[5-(3,3- difluoroazetidin-1-yl)-1H-1,2,3- triazol-1-yl]acetyl}-4-fluoro-N- [(S)-[6-fluoro-5-(propan-2- yl)pyridin-2- yl](phenyl)methyl]pyrrolidine-2- carboxamide |
| GZ-808 | 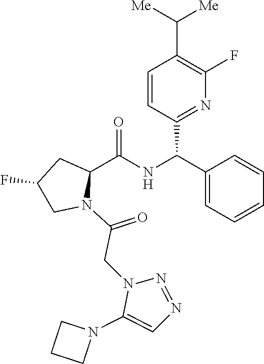 [SMILES: CC(C)c1ccc([C@@H](NC(=O)[C@@H]2C[C@@H](F)CN2C(=O)Cn2nncc2N2CCC2)c2ccccc2)nc1F] | (2S,4R)-1-{2-[5-(azetidin-1-yl)-1H- 1,2,3-triazol-1-yl]acetyl}-4-fluoro- N-[(S)-[6-fluoro-5-(propan-2- yl)pyridin-2- yl](phenyl)methyl]pyrrolidine-2- carboxamide |
| GZ-809 | 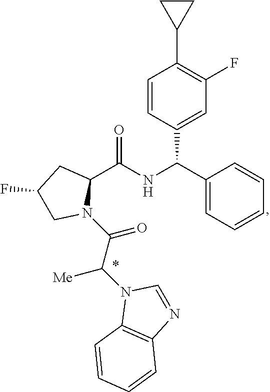 [SMILES: CC(C(=O)N1C[C@H](F)C[C@H]1C(=O)N[C@@H](c1ccccc1)c1ccc(C2CC2)c(F)c1)n1cnc2ccccc21] | (2S,4R)-1-[(2S) or (2R)-2-(1H-1,3- benzodiazol-1-yl)propanoyl]-N- [(S)-(4-cyclopropyl-3- fluorophenyl)(phenyl)methyl]-4- fluoropyrrolidine-2-carboxamide |
| GZ-810 | 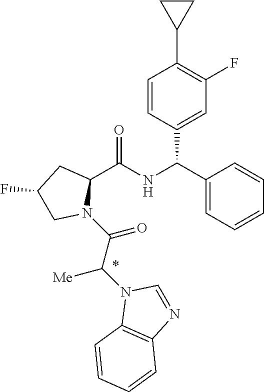 [SMILES: CC(C(=O)N1C[C@H](F)C[C@H]1C(=O)N[C@@H](c1ccccc1)c1ccc(C2CC2)c(F)c1)n1cnc2ccccc21] | (2S,4R)-1-[(2R) or (2S)-2-(1H-1,3- benzodiazol-1-yl)propanoyl]-N- [(S)-(4-cyclopropyl-3- fluorophenyl)(phenyl)methyl]-4- fluoropyrrolidine-2-carboxamide |

In some embodiments, provided herein is a compound of formula (I), or any variation or embodiment thereof, or a stereoisomer or tautomer thereof, or a pharmaceutically acceptable salt of any of the foregoing, the compound, or a stereoisomer or tautomer thereof, or a pharmaceutically acceptable salt of any of the foregoing, wherein (G1-Z1) and (G2-Z2) are each, independently, a moiety selected from the group consisting of:
1-cyclopropanecarbonyl-N—{phenyl[4-(propan-2-yl)phenyl]methyl}pyrrolidine-2-carboxamide;
1-acetyl-4-hydroxy-N—{phenyl[4-(propan-2-yl)phenyl]methyl}pyrrolidine-2-carboxamide;
1-acetyl-3-hydroxy-N—{phenyl[4-(propan-2-yl)phenyl]methyl}pyrrolidine-2-carboxamide;
1-acetyl-4-fluoro-N—{phenyl[4-(propan-2-yl)phenyl]methyl}pyrrolidine-2-carboxamide;
1-acetyl-N—{phenyl[15-(propan-2-yl)pyridin-2-yl]methyl}pyrrolidine-2-carboxamide;
1-(3-carbamoyl-2-acetamidopropanoyl)-4-fluoro-N—{phenyl[4-(propan-2-yl)phenyl]methyl}pyrrolidine-2-carboxamide;
tert-butyl N-{2-oxo-2-[2-({phenyl[4-(propan-2-yl)phenyl]methyl}carbamoyl)pyrrolidin-1-yl]ethyl}carbamate;
1-(2-hydroxyacetyl)-N-{phenyl[4-(propan-2-yl)phenyl]methyl}pyrrolidine-2-carboxamide;
1-(2-acetamidoacetyl)-N-{phenyl[4-(propan-2-yl)phenyl]methyl}pyrrolidine-2-carboxamide;
1-acetyl-N-[(4-cyclopropyl-3-fluorophenyl)(phenyl)methyl]-4-fluoropyrrolidine-2-carboxamide;
1-(3-carbamoylpropanoyl)-N-{phenyl[4-(propan-2-yl)phenyl]methyl}pyrrolidine-2-carboxamide;
1-acetyl-N-[(5-cyclopropylpyridin-2-yl)(phenyl)methyl]-4-fluoropyrrolidine-2-carboxamide;
2-acetyl-N-{phenyl[4-(propan-2-yl)phenyl]methyl}-2-azabicyclo[3.1.0]hexane-3-carboxamide;
1-{2-[N-(carbamoylmethyl)acetamido]acetyl}-4-fluoro-N-{phenyl[4-(propan-2-yl)phenyl]methyl}pyrrolidine-2-carboxamide;
1-acetyl-5-methyl-N-{phenyl[4-(propan-2-yl)phenyl]methyl}pyrrolidine-2-carboxamide;
1-[2-(1,3-oxazol-2-yl)acetyl]-N-{phenyl[4-(propan-2-yl)phenyl]methyl}pyrrolidine-2-carboxamide;
4-fluoro-N-{phenyl[4-(propan-2-yl)phenyl]methyl}-1-[2-(4H-1,2,4-triazol-3-yl)acetyl]pyrrolidine-2-carboxamide;
4-fluoro-1-{2-[(3-methyloxetan-3-yl)amino]acetyl}-N-{phenyl[4-(propan-2-yl)phenyl]methyl}pyrrolidine-2-carboxamide;
4-acetamido-5-[4-fluoro-2-({phenyl[4-(propan-2-yl)phenyl]methyl}carbamoyl)pyrrolidin-1-yl]-5-oxopentanoic acid;
4-fluoro-N-{phenyl[4-(propan-2-yl)phenyl]methyl}-1-[2-(1H-1,2,3-triazol-5-yl)acetyl]pyrrolidine-2-carboxamide;
1-(3-acetamidopropanoyl)-4-fluoro-N-{phenyl[4-(propan-2-yl)phenyl]methyl}pyrrolidine-2-carboxamide;
4-fluoro-1-[2-(2-oxopyrrolidin-1-yl)acetyl]-N-{phenyl[4-(propan-2-yl)phenyl]methyl}pyrrolidine-2-carboxamide;
4-fluoro-1-[2-(1,3-oxazol-5-yl)acetyl]-N-{phenyl[4-(propan-2-yl)phenyl]methyl}pyrrolidine-2-carboxamide;
1-(4-acetamidobutanoyl)-4-fluoro-N-{phenyl[4-(propan-2-yl)phenyl]methyl}pyrrolidine-2-carboxamide;
2-acetyl-N-[(4-cyclopropyl-3-fluorophenyl)(phenyl)methyl]-2-azabicyclo[3.1.0]hexane-3-carboxamide;
N-[(4-cyclopropyl-3-fluorophenyl)(phenyl)methyl]-1-(2-acetamidoacetyl)pyrrolidine-2-carboxamide;
3-acetyl-N-{phenyl[4-(propan-2-yl)phenyl]methyl}-3-azabicyclo[3.1.0]hexane-2-carboxamide;
4-fluoro-1-[2-(2-oxo-1,3-oxazolidin-3-yl)acetyl]-N-{phenyl[4-(propan-2-yl)phenyl]methyl}pyrrolidine-2-carboxamide;
4-fluoro-1-[2-(oxetan-3-yl)acetyl]-N-{phenyl[4-(propan-2-yl)phenyl]methyl}pyrrolidine-2-carboxamide;
4-fluoro-1-[2-(3-oxomorpholin-4-yl)acetyl]-N-{phenyl[4-(propan-2-yl)phenyl]methyl}pyrrolidine-2-carboxamide;
1-acetyl-N-[(4-cyclopropyl-3-fluorophenyl)(phenyl)methyl]-5-methylpyrrolidine-2-carboxamide;
4-fluoro-1-[2-(1-methyl-1H-pyrazol-5-yl)acetyl]-N-{phenyl[4-(propan-2-yl)phenyl]methyl}pyrrolidine-2-carboxamide;
N-[(4-cyclopropyl-3-fluorophenyl)(phenyl)methyl]-4-fluoro-1-[2-(1H-pyrazol-1-yl)acetyl]pyrrolidine-2-carboxamide;
N-[(4-cyclopropyl-3-fluorophenyl)(phenyl)methyl]-4-fluoro-1-[2-(3-oxomorpholin-4-yl)acetyl]pyrrolidine-2-carboxamide;
N-[(4-cyclopropyl-3-fluorophenyl)(phenyl)methyl]-4-fluoro-1-[2-(5-methyl-4H-1,2,4-triazol-3-yl)acetyl]pyrrolidine-2-carboxamide;
N-[(4-cyclopropyl-3-fluorophenyl)(phenyl)methyl]-4-fluoro-1-[3-(1,3-oxazol-2-yl)propanoyl]pyrrolidine-2-carboxamide;
N-[(4-cyclopropyl-3-fluorophenyl)(phenyl)methyl]-4-fluoro-1-[2-(1H-1,2,3,4-tetrazol-1-yl)acetyl]pyrrolidine-2-carboxamide;
N-[(4-cyclopropyl-3-fluorophenyl)(phenyl)methyl]-4-fluoro-1-[2-(2-oxo-1,3-oxazolidin-3-yl)acetyl]pyrrolidine-2-carboxamide;
N-[(4-cyclopropyl-3-fluorophenyl)(phenyl)methyl]-4-fluoro-1-[2-(1H-pyrazol-5-yl)acetyl]pyrrolidine-2-carboxamide;
N-[(4-cyclopropyl-3-fluorophenyl)(phenyl)methyl]-4-fluoro-1-[2-(1H-1,2,3,4-tetrazol-5-yl)acetyl]pyrrolidine-2-carboxamide;
N-[(4-cyclopropyl-3-fluorophenyl)(phenyl)methyl]-4-fluoro-1-[2-(1,2-oxazol-3-yl)acetyl]pyrrolidine-2-carboxamide;
4-fluoro-N-{phenyl[4-(propan-2-yl)phenyl]methyl}-1-[2-(1H-pyrazol-1-yl)acetyl]pyrrolidine-2-carboxamide;
4-fluoro-N-{phenyl[4-(propan-2-yl)phenyl]methyl}-1-[2-(1H-1,2,3-triazol-1-yl)acetyl]pyrrolidine-2-carboxamide;
4-fluoro-N-{phenyl[4-(propan-2-yl)phenyl]methyl}-1-[2-(2H-1,2,3,4-tetrazol-5-yl)acetyl]pyrrolidine-2-carboxamide;
4-fluoro-1-[2-(1,3,4-oxadiazol-2-yl)acetyl]-N-{phenyl[4-(propan-2-yl)phenyl]methyl}pyrrolidine-2-carboxamide;
N-[(4-cyclopropyl-3-fluorophenyl)(phenyl)methyl]-4-fluoro-1-[2-(1H-pyrazol-4-yl)acetyl]pyrrolidine-2-carboxamide;
4-fluoro-1-{2-[(1,3-oxazol-2-yl)amino]acetyl}-N-{phenyl[4-(propan-2-yl)phenyl]methyl}pyrrolidine-2-carboxamide;
N-[(4-cyclopropyl-3-fluorophenyl)(phenyl)methyl]-4-fluoro-1-[2-(3-methyl-2-oxoimidazolidin-1-yl)acetyl]pyrrolidine-2-carboxamide;
1-[2-(5-cyclopropyl-1,3,4-oxadiazol-2-yl)acetyl]-N-[(4-cyclopropyl-3-fluorophenyl)(phenyl)methyl]-4-fluoropyrrolidine-2-carboxamide;
N-[(4-cyclopropyl-3-fluorophenyl)(phenyl)methyl]-4-fluoro-1-{2-[5-(trifluoromethyl)-1,3,4-oxadiazol-2-yl]acetyl}pyrrolidine-2-carboxamide;
N-[(4-cyclopropyl-3-fluorophenyl)(phenyl)methyl]-4-fluoro-1-[2-(1H-1,2,3-triazol-5-yl)acetyl]pyrrolidine-2-carboxamide;
N-[(4-cyclopropyl-3-fluorophenyl)(phenyl)methyl]-1-{2-[5-(difluoromethyl)-1,3,4-oxadiazol-2-yl]acetyl}-4-fluoropyrrolidine-2-carboxamide;
4-fluoro-N-{phenyl[4-(propan-2-yl)phenyl]methyl}-1-[3-(1H-1,2,4-triazol-1-yl)propanoyl]pyrrolidine-2-carboxamide;
4-fluoro-1-[2-(1-methyl-1H-pyrazol-4-yl)acetyl]-N-{phenyl[4-(propan-2-yl)phenyl]methyl}pyrrolidine-2-carboxamide;
4-fluoro-N-{phenyl[4-(propan-2-yl)phenyl]methyl}-1-[2-(1H-1,2,3,4-tetrazol-1-yl)acetyl]pyrrolidine-2-carboxamide;
4-fluoro-N-{phenyl[4-(propan-2-yl)phenyl]methyl}-1-[2-(pyridazin-3-yloxy)acetyl]pyrrolidine-2-carboxamide;
4-fluoro-1-(1-methyl-5-oxopyrrolidine-2-carbonyl)-N-{phenyl[4-(propan-2-yl)phenyl]methyl}pyrrolidine-2-carboxamide;
1-[2-(2-chloro-5-fluorophenyl)acetyl]-4-fluoro-N-{phenyl[4-(propan-2-yl)phenyl]methyl}pyrrolidine-2-carboxamide;
4-fluoro-N-{phenyl[4-(propan-2-yl)phenyl]methyl}-1-[4-(pyridin-3-yl)butanoyl]pyrrolidine-2-carboxamide;
4-fluoro-1-(3-oxo-octahydroindolizine-6-carbonyl)-N-{phenyl[4-(propan-2-yl)phenyl]methyl}pyrrolidine-2-carboxamide;
4-fluoro-1-(2-oxopiperidine-4-carbonyl)-N-{phenyl[4-(propan-2-yl)phenyl]methyl}pyrrolidine-2-carboxamide;
1-[2-(2-cyano-4-methoxyphenyl)acetyl]-4-fluoro-N-{phenyl[4-(propan-2-yl)phenyl]methyl}pyrrolidine-2-carboxamide;
4-fluoro-1-{3-oxo-2H,3H-[1,2,4]triazolo[4,3-a]pyridine-8-carbonyl}-N-{phenyl[4-(propan-2-yl)phenyl]methyl}pyrrolidine-2-carboxamide;
4-fluoro-1-[4-(1H-imidazol-1-yl)butanoyl]-N-{phenyl[4-(propan-2-yl)phenyl]methyl}pyrrolidine-2-carboxamide;
4-fluoro-N-{phenyl[4-(propan-2-yl)phenyl]methyl}-1-[2-(pyrimidin-5-yl)acetyl]pyrrolidine-2-carboxamide;
4-fluoro-1-(4-methylpyrimidine-5-carbonyl)-N-{phenyl[4-(propan-2-yl)phenyl]methyl}pyrrolidine-2-carboxamide;
4-fluoro-1-[2-(2-oxo-1,2-dihydropyridin-1-yl)acetyl]-N-{phenyl[4-(propan-2-yl)phenyl]methyl}pyrrolidine-2-carboxamide;
1-[3-(3,5-dimethyl-1,2-oxazol-4-yl)propanoyl]-4-fluoro-N-{phenyl[4-(propan-2-yl)phenyl]methyl}pyrrolidine-2-carboxamide;
4-fluoro-1-[2-(3-fluoro-4-methoxyphenyl)acetyl]-N-{phenyl[4-(propan-2-yl)phenyl]methyl}pyrrolidine-2-carboxamide;
4-fluoro-1-(1,3-oxazole-5-carbonyl)-N-{phenyl[4-(propan-2-yl)phenyl]methyl}pyrrolidine-2-carboxamide;
4-fluoro-N-{phenyl[4-(propan-2-yl)phenyl]methyl}-1-[4-(pyridin-4-yl)butanoyl]pyrrolidine-2-carboxamide;
1-[(dimethylcarbamoyl)carbonyl]-4-fluoro-N-{phenyl[4-(propan-2-yl)phenyl]methyl}pyrrolidine-2-carboxamide;
4-fluoro-1-[2-(5-methyl-2,4-dioxo-1,2,3,4-tetrahydropyrimidin-1-yl)acetyl]-N-{phenyl[4-(propan-2-yl)phenyl]methyl}pyrrolidine-2-carboxamide;
4-fluoro-1-[2-(1H-imidazol-1-yl)acetyl]-N-{phenyl[4-(propan-2-yl)phenyl]methyl}pyrrolidine-2-carboxamide;
4-fluoro-N-{phenyl[4-(propan-2-yl)phenyl]methyl}-1-[2-(1H-pyrazol-5-yl)acetyl]pyrrolidine-2-carboxamide;
4-fluoro-1-[2-methyl-3-(1H-1,2,4-triazol-1-yl)propanoyl]-N-{phenyl[4-(propan-2-yl)phenyl]methyl}pyrrolidine-2-carboxamide;
4-fluoro-1-[2-(5-fluoropyridin-2-yl)acetyl]-N-{phenyl[4-(propan-2-yl)phenyl]methyl}pyrrolidine-2-carboxamide;
4-fluoro-1-[2-(5-methyl-1H-indol-3-yl)acetyl]-N-{phenyl[4-(propan-2-yl)phenyl]methyl}pyrrolidine-2-carboxamide;
1-[2-(4-acetamidophenyl)acetyl]-4-fluoro-N-{phenyl[4-(propan-2-yl)phenyl]methyl}pyrrolidine-2-carboxamide;
4-fluoro-1-[2-(1H-imidazol-4-yl)acetyl]-N-{phenyl[4-(propan-2-yl)phenyl]methyl}pyrrolidine-2-carboxamide;
4-fluoro-N-{phenyl[4-(propan-2-yl)phenyl]methyl}-1-[3-(pyridin-3-yl)propanoyl]pyrrolidine-2-carboxamide;
4-fluoro-1-[2-(4-methoxyphenyl)acetyl]-N-{phenyl[4-(propan-2-yl)phenyl]methyl}pyrrolidine-2-carboxamide;
1-[2-(1,5-dimethyl-1H-pyrazol-3-yl)acetyl]-4-fluoro-N-{phenyl[4-(propan-2-yl)phenyl]methyl}pyrrolidine-2-carboxamide;
4-fluoro-1-[3-(2-methylpyridin-3-yl)propanoyl]-N-{phenyl[4-(propan-2-yl)phenyl]methyl}pyrrolidine-2-carboxamide;
4-fluoro-N-{phenyl[4-(propan-2-yl)phenyl]methyl}-1-[2-(pyrimidin-2-yl)acetyl]pyrrolidine-2-carboxamide;
4-fluoro-N-{phenyl[4-(propan-2-yl)phenyl]methyl}-1-[3-(pyrazin-2-yl)propanoyl]pyrrolidine-2-carboxamide;
4-fluoro-N-{phenyl[4-(propan-2-yl)phenyl]methyl}-1-[2-(2H-1,2,3-triazol-2-yl)acetyl]pyrrolidine-2-carboxamide;
1-[3-(2,6-dimethylpyridin-3-yl)propanoyl]-4-fluoro-N-{phenyl[4-(propan-2-yl)phenyl]methyl}pyrrolidine-2-carboxamide;
1-[2-(1H-1,2,3-benzotriazol-1-yl)acetyl]-4-fluoro-N-{phenyl[4-(propan-2-yl)phenyl]methyl}pyrrolidine-2-carboxamide;
4-fluoro-N-{phenyl[4-(propan-2-yl)phenyl]methyl}-1-[2-(1,3,5-trimethyl-1H-pyrazol-4-yl)acetyl]pyrrolidine-2-carboxamide;
4-fluoro-N-{phenyl[4-(propan-2-yl)phenyl]methyl}-1-[2-(pyridin-3-yloxy)acetyl]pyrrolidine-2-carboxamide;
4-fluoro-1-[3-(1H-imidazol-5-yl)propanoyl]-N-{phenyl[4-(propan-2-yl)phenyl]methyl}pyrrolidine-2-carboxamide;
4-fluoro-1-[3-(5-methyl-1,3,4-thiadiazol-2-yl)propanoyl]-N-{phenyl[4-(propan-2-yl)phenyl]methyl}pyrrolidine-2-carboxamide;
1-[3-(3,5-dimethyl-1H-pyrazol-1-yl)propanoyl]-4-fluoro-N-{phenyl[4-(propan-2-yl)phenyl]methyl}pyrrolidine-2-carboxamide;
1-(3-cyanopropanoyl)-4-fluoro-N-{phenyl[4-(propan-2-yl)phenyl]methyl}pyrrolidine-2-carboxamide;
1-{2-[(dimethylcarbamoyl)amino]acetyl}-4-fluoro-N-{phenyl[4-(propan-2-yl)phenyl]methyl}pyrrolidine-2-carboxamide;
4-fluoro-1-[2-(5-methyl-1,2,4-oxadiazol-3-yl)acetyl]-N-{phenyl[4-(propan-2-yl)phenyl]methyl}pyrrolidine-2-carboxamide;
4-fluoro-1-[2-(1-methyl-1H-indol-2-yl)acetyl]-N-{phenyl[4-(propan-2-yl)phenyl]methyl}pyrrolidine-2-carboxamide;
4-fluoro-1-[3-(5-methylpyridin-2-yl)propanoyl]-N-{phenyl[4-(propan-2-yl)phenyl]methyl}pyrrolidine-2-carboxamide;
1-(2-ethyl-1,3-oxazole-4-carbonyl)-4-fluoro-N-{phenyl[4-(propan-2-yl)phenyl]methyl}pyrrolidine-2-carboxamide;
4-fluoro-1-[2-(2-methyl-1,3-thiazol-4-yl)acetyl]-N-{phenyl[4-(propan-2-yl)phenyl]methyl}pyrrolidine-2-carboxamide;
4-fluoro-N-{phenyl[4-(propan-2-yl)phenyl]methyl}-1-[2-(pyrazin-2-yl)acetyl]pyrrolidine-2-carboxamide;
4-fluoro-1-[2-methyl-3-(1H-pyrazol-1-yl)propanoyl]-N-{phenyl[4-(propan-2-yl)phenyl]methyl}pyrrolidine-2-carboxamide;
4-fluoro-1-[3-(1H-indol-3-yl)propanoyl]-N-{phenyl[4-(propan-2-yl)phenyl]methyl}pyrrolidine-2-carboxamide;
4-fluoro-1-[2-methyl-3-(pyridin-4-yl)propanoyl]-N-{phenyl[4-(propan-2-yl)phenyl]methyl}pyrrolidine-2-carboxamide;
4-fluoro-1-[2-(4-fluoro-1H-indol-1-yl)acetyl]-N-{phenyl[4-(propan-2-yl)phenyl]methyl}pyrrolidine-2-carboxamide;
4-fluoro-1-{4-oxo-4H,5H,6H,7H,8H-pyrazolo[1,5-a][1,4]diazepine-2-carbonyl}-N-{phenyl[4-(propan-2-yl)phenyl]methyl}pyrrolidine-2-carboxamide;
4-fluoro-N-{phenyl[4-(propan-2-yl)phenyl]methyl}-1-{2-[5-(propan-2-yl)-1,2,4-oxadiazol-3-yl]acetyl}pyrrolidine-2-carboxamide;
4-fluoro-1-(6-oxopiperidine-3-carbonyl)-N-{phenyl[4-(propan-2-yl)phenyl]methyl}pyrrolidine-2-carboxamide;
4-fluoro-N-{phenyl[4-(propan-2-yl)phenyl]methyl}-1-[2-(pyrrolidine-1-sulfonyl)acetyl]pyrrolidine-2-carboxamide;
1-[2-(1,2-benzoxazol-3-yl)acetyl]-4-fluoro-N-{phenyl[4-(propan-2-yl)phenyl]methyl}pyrrolidine-2-carboxamide;
4-fluoro-N-{phenyl[4-(propan-2-yl)phenyl]methyl}-1-(2-{[1,2,4]triazolo[1,5-a]pyridin-6-yl}acetyl)pyrrolidine-2-carboxamide;
1-[2-(1H-1,3-benzodiazol-1-yl)acetyl]-4-fluoro-N-{phenyl[4-(propan-2-yl)phenyl]methyl}pyrrolidine-2-carboxamide;
4-fluoro-N-{phenyl[4-(propan-2-yl)phenyl]methyl}-1-[2-(1H-pyrazol-1-yl)propanoyl]pyrrolidine-2-carboxamide;
4-fluoro-1-[3-(3-methoxypyridin-2-yl)propanoyl]-N-{phenyl[4-(propan-2-yl)phenyl]methyl}pyrrolidine-2-carboxamide;
4-fluoro-1-[2-(1H-imidazol-1-yl)propanoyl]-N-{phenyl[4-(propan-2-yl)phenyl]methyl}pyrrolidine-2-carboxamide;
1-[2-(3,5-dimethyl-1,2-oxazol-4-yl)acetyl]-4-fluoro-N-{phenyl[4-(propan-2-yl)phenyl]methyl}pyrrolidine-2-carboxamide;
4-fluoro-1-[2-(1-methyl-1H-pyrazol-3-yl)acetyl]-N-{phenyl[4-(propan-2-yl)phenyl]methyl}pyrrolidine-2-carboxamide;
4-fluoro-N-{phenyl[4-(propan-2-yl)phenyl]methyl}-1-[3-(1H-1,2,3-triazol-1-yl)propanoyl]pyrrolidine-2-carboxamide;
4-fluoro-1-[3-(1H-imidazol-2-yl)propanoyl]-N-{phenyl[4-(propan-2-yl)phenyl]methyl}pyrrolidine-2-carboxamide;
4-fluoro-1-[2-(2-methylphenoxy)acetyl]-N-{phenyl[4-(propan-2-yl)phenyl]methyl}pyrrolidine-2-carboxamide;
4-fluoro-1-[2-(3-methyl-1,2-oxazol-5-yl)acetyl]-N-{phenyl[4-(propan-2-yl)phenyl]methyl}pyrrolidine-2-carboxamide;
4-fluoro-1-(3-methyloxetane-3-carbonyl)-N-{phenyl[4-(propan-2-yl)phenyl]methyl}pyrrolidine-2-carboxamide;
1-[2-(4-chloro-TH-pyrazol-1-yl)acetyl]-4-fluoro-N-{phenyl[4-(propan-2-yl)phenyl]methyl}pyrrolidine-2-carboxamide;
1-(1-ethyl-TH-pyrazole-5-carbonyl)-4-fluoro-N-{phenyl[4-(propan-2-yl)phenyl]methyl}pyrrolidine-2-carboxamide;
4-fluoro-N-{phenyl[4-(propan-2-yl)phenyl]methyl}-1-[2-(pyridin-2-yl)propanoyl]pyrrolidine-2-carboxamide;
4-fluoro-N-{phenyl[4-(propan-2-yl)phenyl]methyl}-1-[3-(pyrimidin-5-yl)propanoyl]pyrrolidine-2-carboxamide;
4-fluoro-1-(3-methoxy-1-methyl-TH-pyrazole-4-carbonyl)-N-{phenyl[4-(propan-2-yl)phenyl]methyl}pyrrolidine-2-carboxamide;
4-fluoro-1-[2-(5-methyl-1,3,4-oxadiazol-2-yl)acetyl]-N-{phenyl[4-(propan-2-yl)phenyl]methyl}pyrrolidine-2-carboxamide;
4-fluoro-N-{phenyl[4-(propan-2-yl)phenyl]methyl}-1-[3-(1H-pyrazol-4-yl)propanoyl]pyrrolidine-2-carboxamide;
4-fluoro-N-{phenyl[4-(propan-2-yl)phenyl]methyl}-1-[2-(1,3-thiazol-4-yl)acetyl]pyrrolidine-2-carboxamide;
4-fluoro-1-[4-(2-methyl-1H-imidazol-1-yl)butanoyl]-N-{phenyl[4-(propan-2-yl)phenyl]methyl}pyrrolidine-2-carboxamide;
4-fluoro-1-(2-{imidazo[1,2-a]pyridin-3-yl}acetyl)-N-{phenyl[4-(propan-2-yl)phenyl]methyl}pyrrolidine-2-carboxamide;
4-fluoro-N-{phenyl[4-(propan-2-yl)phenyl]methyl}-1-[3-(pyridin-2-yl)propanoyl]pyrrolidine-2-carboxamide;
4-fluoro-1-[2-(3-methyl-1,2,4-oxadiazol-5-yl)acetyl]-N-{phenyl[4-(propan-2-yl)phenyl]methyl}pyrrolidine-2-carboxamide;
1-[2-(3,5-dimethyl-1H-pyrazol-1-yl)acetyl]-4-fluoro-N-{phenyl[4-(propan-2-yl)phenyl]methyl}pyrrolidine-2-carboxamide;
4-fluoro-1-[3-(6-methylpyridin-3-yl)propanoyl]-N-{phenyl[4-(propan-2-yl)phenyl]methyl}pyrrolidine-2-carboxamide;
4-fluoro-N-{phenyl[4-(propan-2-yl)phenyl]methyl}-1-(3-{1H-pyrrolo[2,3-b]pyridin-3-yl}propanoyl)pyrrolidine-2-carboxamide;
4-fluoro-1-(2-oxo-1,3-oxazolidine-5-carbonyl)-N-{phenyl[4-(propan-2-yl)phenyl]methyl}pyrrolidine-2-carboxamide;
4-fluoro-1-[2-(4-methyl-1H-pyrazol-1-yl)acetyl]-N-{phenyl[4-(propan-2-yl)phenyl]methyl}pyrrolidine-2-carboxamide;
4-fluoro-1-[3-(1-methyl-1H-pyrazol-4-yl)propanoyl]-N-{phenyl[4-(propan-2-yl)phenyl]methyl}pyrrolidine-2-carboxamide;
4-fluoro-N-{phenyl[4-(propan-2-yl)phenyl]methyl}-1-[2-(1H-1,2,4-triazol-1-yl)acetyl]pyrrolidine-2-carboxamide;
4-fluoro-N-{phenyl[4-(propan-2-yl)phenyl]methyl}-1-[5-(pyridin-4-yl)-1H-pyrazole-3-carbonyl]pyrrolidine-2-carboxamide;
4-fluoro-1-[3-(2-methylpyridin-4-yl)propanoyl]-N-{phenyl[4-(propan-2-yl)phenyl]methyl}pyrrolidine-2-carboxamide;
4-fluoro-1-(2-hydroxy-3-methylbutanoyl)-N-{phenyl[4-(propan-2-yl)phenyl]methyl}pyrrolidine-2-carboxamide;
4-fluoro-N-{phenyl[4-(propan-2-yl)phenyl]methyl}-1-(3-{1H-pyrrolo[2,3-b]pyridin-5-yl}propanoyl)pyrrolidine-2-carboxamide;
4-fluoro-1-[4-oxo-4-(pyrrolidin-1-yl)butanoyl]-N-{phenyl[4-(propan-2-yl)phenyl]methyl}pyrrolidine-2-carboxamide;
4-fluoro-N-{phenyl[4-(propan-2-yl)phenyl]methyl}-1-[2-(quinolin-6-yl)acetyl]pyrrolidine-2-carboxamide;
4-fluoro-N-{phenyl[4-(propan-2-yl)phenyl]methyl}-1-[2-(pyridin-4-yl)acetyl]pyrrolidine-2-carboxamide;
4-fluoro-N-{phenyl[4-(propan-2-yl)phenyl]methyl}-1-[2-(2,2,2-trifluoroacetamido)acetyl]pyrrolidine-2-carboxamide;
4-fluoro-N-{phenyl[4-(propan-2-yl)phenyl]methyl}-1-[2-(pyridin-3-yloxy)propanoyl]pyrrolidine-2-carboxamide;
4-fluoro-1-[2-(1H-indol-3-yl)acetyl]-N-{phenyl[4-(propan-2-yl)phenyl]methyl}pyrrolidine-2-carboxamide;
1-(2-cyclopropyl-2-oxoacetyl)-4-fluoro-N-{phenyl[4-(propan-2-yl)phenyl]methyl}pyrrolidine-2-carboxamide;
4-fluoro-1-[2-(5-fluoro-2-methoxyphenyl)acetyl]-N-{phenyl[4-(propan-2-yl)phenyl]methyl}pyrrolidine-2-carboxamide;
4-fluoro-1-[2-(6-methoxypyridin-2-yl)acetyl]-N-{phenyl[4-(propan-2-yl)phenyl]methyl}pyrrolidine-2-carboxamide;
4-fluoro-1-[2-(2-oxo-1,2-dihydropyridin-1-yl)propanoyl]-N-{phenyl[4-(propan-2-yl)phenyl]methyl}pyrrolidine-2-carboxamide;
4-fluoro-N-{phenyl[4-(propan-2-yl)phenyl]methyl}-1-[2-(pyridin-3-yl)acetyl]pyrrolidine-2-carboxamide;
1-[2-(3,5-dimethyl-1H-pyrazol-4-yl)acetyl]-4-fluoro-N-{phenyl[4-(propan-2-yl)phenyl]methyl}pyrrolidine-2-carboxamide;
1-[2-(2,5-dioxoimidazolidin-1-yl)acetyl]-4-fluoro-N-{phenyl[4-(propan-2-yl)phenyl]methyl}pyrrolidine-2-carboxamide;
1-[2-(2,5-dimethyl-1,3-thiazol-4-yl)acetyl]-4-fluoro-N-{phenyl[4-(propan-2-yl)phenyl]methyl}pyrrolidine-2-carboxamide;
4-fluoro-1-[2-methyl-3-(pyridin-2-yl)propanoyl]-N-{phenyl[4-(propan-2-yl)phenyl]methyl}pyrrolidine-2-carboxamide;
4-fluoro-1-[2-(2-oxo-1,2-dihydropyrazin-1-yl)acetyl]-N-{phenyl[4-(propan-2-yl)phenyl]methyl}pyrrolidine-2-carboxamide;
4-fluoro-1-[2-(N-methylacetamido)propanoyl]-N-{phenyl[4-(propan-2-yl)phenyl]methyl}pyrrolidine-2-carboxamide;
4-fluoro-1-[2-methyl-2-(pyridin-2-yl)propanoyl]-N-{phenyl[4-(propan-2-yl)phenyl]methyl}pyrrolidine-2-carboxamide;
4-fluoro-1-[2-(2-oxopiperidin-1-yl)acetyl]-N-{phenyl[4-(propan-2-yl)phenyl]methyl}pyrrolidine-2-carboxamide;
4-fluoro-N-{phenyl[4-(propan-2-yl)phenyl]methyl}-1-[2-(quinolin-5-yl)acetyl]pyrrolidine-2-carboxamide;
4-fluoro-N-{phenyl[4-(propan-2-yl)phenyl]methyl}-1-[3-(1H-1,2,4-triazol-1-yl)benzoyl]pyrrolidine-2-carboxamide;
4-fluoro-1-{[(2-methylpropyl)carbamoyl]carbonyl}-N-{phenyl[4-(propan-2-yl)phenyl]methyl}pyrrolidine-2-carboxamide;
4-fluoro-N-{phenyl[4-(propan-2-yl)phenyl]methyl}-1-[2-(1H-1,2,3,4-tetrazol-1-yl)propanoyl]pyrrolidine-2-carboxamide;
4-fluoro-1-(2-oxo-1,2,3,4-tetrahydroquinoline-7-carbonyl)-N-{phenyl[4-(propan-2-yl)phenyl]methyl}pyrrolidine-2-carboxamide;
4-fluoro-1-[2-(2-methyl-1,3-thiazol-5-yl)acetyl]-N-{phenyl[4-(propan-2-yl)phenyl]methyl}pyrrolidine-2-carboxamide;
4-fluoro-1-[3-(6-oxo-1,6-dihydropyridazin-3-yl)propanoyl]-N-{phenyl[4-(propan-2-yl)phenyl]methyl}pyrrolidine-2-carboxamide;
4-fluoro-1-[2-(4-methyl-1H-pyrazol-1-yl)propanoyl]-N-{phenyl[4-(propan-2-yl)phenyl]methyl}pyrrolidine-2-carboxamide;
4-fluoro-N-{phenyl[4-(propan-2-yl)phenyl]methyl}-1-[2-(pyridin-2-yl)acetyl]pyrrolidine-2-carboxamide;
4-fluoro-1-[2-(3-methyl-1H-pyrazol-1-yl)acetyl]-N-{phenyl[4-(propan-2-yl)phenyl]methyl}pyrrolidine-2-carboxamide;
4-fluoro-1-(3-oxo-3,4-dihydro-2H-1,4-benzoxazine-6-carbonyl)-N-{phenyl[4-(propan-2-yl)phenyl]methyl}pyrrolidine-2-carboxamide;
1-(2-acetamidopyridine-4-carbonyl)-4-fluoro-N-{phenyl[4-(propan-2-yl)phenyl]methyl}pyrrolidine-2-carboxamide;
4-fluoro-N-{phenyl[4-(propan-2-yl)phenyl]methyl}-1-[2-(1H-pyrazol-3-yl)acetyl]pyrrolidine-2-carboxamide;
N-{[3-fluoro-4-(propan-2-yl)phenyl](phenyl)methyl}-3-[2-(1H-1,2,3-triazol-5-yl)acetyl]-3-azabicyclo[3.1.0]hexane-2-carboxamide;
N-[(4-cyclopropyl-3-fluorophenyl)(phenyl)methyl]-4-fluoro-1-[2-(5-methyl-1H-1,2,3,4-tetrazol-1-yl)acetyl]pyrrolidine-2-carboxamide;
N-[(4-cyclopropyl-3-fluorophenyl)(phenyl)methyl]-4-fluoro-1-[2-(1,3,4-oxadiazol-2-yl)acetyl]pyrrolidine-2-carboxamide;
4-fluoro-N-{phenyl[5-(propan-2-yl)pyridin-2-yl]methyl}-1-[2-(1H-1,2,3-triazol-5-yl)acetyl]pyrrolidine-2-carboxamide;
4-fluoro-N-{[3-fluoro-4-(propan-2-yl)phenyl](phenyl)methyl}-1-[2-(1H-1,2,3-triazol-5-yl)acetyl]pyrrolidine-2-carboxamide;
N-{[3-fluoro-4-(propan-2-yl)phenyl](phenyl)methyl}-2-[2-(1H-1,2,3-triazol-5-yl)acetyl]-2-azabicyclo[3.1.0]hexane-3-carboxamide;
N-{[3-fluoro-4-(propan-2-yl)phenyl](phenyl)methyl}-5-methyl-1-[2-(1H-1,2,3-triazol-5-yl)acetyl]pyrrolidine-2-carboxamide;
4-fluoro-N-{[3-fluoro-4-(propan-2-yl)phenyl](phenyl)methyl}-1-(2-hydroxy-2-methylpropanoyl)pyrrolidine-2-carboxamide;
4-fluoro-1-[2-(5-methyl-1H-1,2,3,4-tetrazol-1-yl)acetyl]-N-{phenyl[4-(propan-2-yl)phenyl]methyl}pyrrolidine-2-carboxamide;
4-fluoro-N-{[3-fluoro-4-(propan-2-yl)phenyl](phenyl)methyl}-1-[2-(1H-1,2,3,4-tetrazol-5-yl)acetyl]pyrrolidine-2-carboxamide;
N-[(4-cyclopropyl-3-fluorophenyl)(phenyl)methyl]-4-fluoro-1-[2-(5-methyl-2H-1,2,3,4-tetrazol-2-yl)acetyl]pyrrolidine-2-carboxamide;
N-[(4-cyclopropyl-3-fluorophenyl)(phenyl)methyl]-3-[2-(1H-1,2,3-triazol-5-yl)acetyl]-3-azabicyclo[3.1.0]hexane-2-carboxamide;
4-fluoro-N-{[3-fluoro-4-(propan-2-yl)phenyl](phenyl)methyl}-1-[2-(1H-1,2,3-triazol-1-yl)acetyl]pyrrolidine-2-carboxamide;
4-fluoro-N-{[3-fluoro-4-(propan-2-yl)phenyl](phenyl)methyl}-1-[2-(1H-1,2,3,4-tetrazol-1-yl)acetyl]pyrrolidine-2-carboxamide;
4-fluoro-N-{phenyl[4-(propan-2-yl)phenyl]methyl}-1-[2-(4H-1,2,4-triazol-4-yl)acetyl]pyrrolidine-2-carboxamide;
4-fluoro-1-[2-(4-methyl-4H-1,2,4-triazol-3-yl)acetyl]-N-{phenyl[4-(propan-2-yl)phenyl]methyl}pyrrolidine-2-carboxamide;
4-fluoro-1-[2-(1-methyl-1H-1,2,3-triazol-5-yl)acetyl]-N-{phenyl[4-(propan-2-yl)phenyl]methyl}pyrrolidine-2-carboxamide;
4-fluoro-1-[2-(1-methyl-1H-1,2,3-triazol-4-yl)acetyl]-N-{phenyl[4-(propan-2-yl)phenyl]methyl}pyrrolidine-2-carboxamide;
4-fluoro-1-[2-(1,2-oxazol-4-yl)acetyl]-N-{phenyl[4-(propan-2-yl)phenyl]methyl}pyrrolidine-2-carboxamide;
4-fluoro-1-[2-(1,2-oxazol-3-yl)acetyl]-N-{phenyl[4-(propan-2-yl)phenyl]methyl}pyrrolidine-2-carboxamide;
4-fluoro-1-[2-(3-methyl-1H-1,2,4-triazol-5-yl)acetyl]-N-{phenyl[4-(propan-2-yl)phenyl]methyl}pyrrolidine-2-carboxamide;
4-fluoro-N-{[3-fluoro-4-(propan-2-yl)phenyl](phenyl)methyl}-1-[2-(1-methyl-1H-pyrazol-5-yl)acetyl]pyrrolidine-2-carboxamide;
4-fluoro-N-{[3-fluoro-4-(propan-2-yl)phenyl](phenyl)methyl}-1-[2-(1-methyl-1H-pyrazol-3-yl)acetyl]pyrrolidine-2-carboxamide;
4-fluoro-N-{[3-fluoro-4-(propan-2-yl)phenyl](phenyl)methyl}-1-[2-(1-methyl-1H-1,2,3-triazol-5-yl)acetyl]pyrrolidine-2-carboxamide;
4-fluoro-N-{[3-fluoro-4-(propan-2-yl)phenyl](phenyl)methyl}-1-[2-(pyridin-2-yl)acetyl]pyrrolidine-2-carboxamide;
4-fluoro-N-{[3-fluoro-4-(propan-2-yl)phenyl](phenyl)methyl}-1-[2-(5-methyl-1,3,4-oxadiazol-2-yl)acetyl]pyrrolidine-2-carboxamide;
4-fluoro-N-{[3-fluoro-4-(propan-2-yl)phenyl](phenyl)methyl}-1-[2-(pyridin-3-yl)acetyl]pyrrolidine-2-carboxamide;
4-fluoro-N-{[3-fluoro-4-(propan-2-yl)phenyl](phenyl)methyl}-1-[2-(pyrimidin-4-yl)acetyl]pyrrolidine-2-carboxamide;
4-fluoro-N-{[3-fluoro-4-(propan-2-yl)phenyl](phenyl)methyl}-1-[2-(pyrimidin-5-yl)acetyl]pyrrolidine-2-carboxamide;
4-fluoro-N-{[3-fluoro-4-(propan-2-yl)phenyl](phenyl)methyl}-1-[2-(pyrazin-2-yl)acetyl]pyrrolidine-2-carboxamide;
4-fluoro-N-{[3-fluoro-4-(propan-2-yl)phenyl](phenyl)methyl}-1-[2-(4-methyl-2,5-dioxopiperazin-1-yl)acetyl]pyrrolidine-2-carboxamide;
1-(2-cyanoacetyl)-4-fluoro-N-{phenyl[4-(propan-2-yl)phenyl]methyl}pyrrolidine-2-carboxamide;
4-fluoro-1-(2-methanesulfonylacetyl)-N-{phenyl[4-(propan-2-yl)phenyl]methyl}pyrrolidine-2-carboxamide;
4-fluoro-N-{phenyl[4-(propan-2-yl)phenyl]methyl}-1-[2-(1H-1,2,3-triazol-1-yl)propanoyl]pyrrolidine-2-carboxamide;
1-(4-acetylmorpholine-2-carbonyl)-4-fluoro-N-{phenyl[4-(propan-2-yl)phenyl]methyl}pyrrolidine-2-carboxamide;
1-[2-(4-acetyl-3,4-dihydro-2H-1,4-benzoxazin-2-yl)acetyl]-4-fluoro-N-{phenyl[4-(propan-2-yl)phenyl]methyl}pyrrolidine-2-carboxamide;
1-[2-(4-acetyl-2-oxopiperazin-1-yl)acetyl]-4-fluoro-N-{phenyl[4-(propan-2-yl)phenyl]methyl}pyrrolidine-2-carboxamide;
1-(1-acetylpiperidine-4-carbonyl)-4-fluoro-N-{phenyl[4-(propan-2-yl)phenyl]methyl}pyrrolidine-2-carboxamide;
1-(2,3-dihydroxypropanoyl)-4-fluoro-N-{phenyl[4-(propan-2-yl)phenyl]methyl}pyrrolidine-2-carboxamide;
1-{8-acetyl-8-azaspiro[4.5]decane-2-carbonyl}-4-fluoro-N-{phenyl[4-(propan-2-yl)phenyl]methyl}pyrrolidine-2-carboxamide;
4-fluoro-1-[3-(1H-imidazol-1-yl)-2-methylpropanoyl]-N-{phenyl[4-(propan-2-yl)phenyl]methyl}pyrrolidine-2-carboxamide;
1-{6-acetyl-6-azaspiro[2.5]octane-1-carbonyl}-4-fluoro-N-{phenyl[4-(propan-2-yl)phenyl]methyl}pyrrolidine-2-carboxamide;
1-(1-acetyl-3-methylpyrrolidine-3-carbonyl)-4-fluoro-N-{phenyl[4-(propan-2-yl)phenyl]methyl}pyrrolidine-2-carboxamide;
4-fluoro-1-[2-(N-methylacetamido)acetyl]-N-{phenyl[4-(propan-2-yl)phenyl]methyl}pyrrolidine-2-carboxamide;
1-{2-acetyl-5-oxa-2,6-diazaspiro[3.4]oct-6-ene-7-carbonyl}-4-fluoro-N-{phenyl[4-(propan-2-yl)phenyl]methyl}pyrrolidine-2-carboxamide;
1-[1-acetyl-2-(pyridin-3-yl)pyrrolidine-3-carbonyl]-4-fluoro-N-{phenyl[4-(propan-2-yl)phenyl]methyl}pyrrolidine-2-carboxamide;
4-fluoro-1-[5-(methoxymethyl)-1,2-oxazole-4-carbonyl]-N-{phenyl[4-(propan-2-yl)phenyl]methyl}pyrrolidine-2-carboxamide;
1-{6-acetyl-5H,6H,7H,8H-pyrido[3,4-b]pyrazine-7-carbonyl}-4-fluoro-N-{phenyl[4-(propan-2-yl)phenyl]methyl}pyrrolidine-2-carboxamide;
4-fluoro-N-{phenyl[4-(propan-2-yl)phenyl]methyl}-1-[2-(1H-1,2,4-triazol-1-yl)propanoyl]pyrrolidine-2-carboxamide;
1-{5-acetyl-5-azaspiro[2.4]heptane-1-carbonyl}-4-fluoro-N-{phenyl[4-(propan-2-yl)phenyl]methyl}pyrrolidine-2-carboxamide;
1-[3-(1-acetylpyrrolidin-2-yl)propanoyl]-4-fluoro-N-{phenyl[4-(propan-2-yl)phenyl]methyl}pyrrolidine-2-carboxamide;
1-{4-[(1-acetylazetidin-3-yl)oxy]benzoyl}-4-fluoro-N-{phenyl[4-(propan-2-yl)phenyl]methyl}pyrrolidine-2-carboxamide;
4-fluoro-1-[3-methoxy-2-(N-methylacetamido)butanoyl]-N-{phenyl[4-(propan-2-yl)phenyl]methyl}pyrrolidine-2-carboxamide;
1-[2-(1-acetylpyrrolidin-2-yl)acetyl]-4-fluoro-N-{phenyl[4-(propan-2-yl)phenyl]methyl}pyrrolidine-2-carboxamide;
1-{7-acetyl-1-oxa-2,7-diazaspiro[4.4]non-2-ene-3-carbonyl}-4-fluoro-N-{phenyl[4-(propan-2-yl)phenyl]methyl}pyrrolidine-2-carboxamide;
1-{5-acetyl-hexahydro-1H-furo[3,4-c]pyrrole-3a-carbonyl}-4-fluoro-N-{phenyl[4-(propan-2-yl)phenyl]methyl}pyrrolidine-2-carboxamide;
1-(1-acetylpyrrolidine-3-carbonyl)-4-fluoro-N-{phenyl[4-(propan-2-yl)phenyl]methyl}pyrrolidine-2-carboxamide;
4-fluoro-1-[2-(3-methyl-1H-pyrazol-5-yl)acetyl]-N-{phenyl[4-(propan-2-yl)phenyl]methyl}pyrrolidine-2-carboxamide;
1-{5-acetyl-2-oxa-5-azabicyclo[2.2.1]heptane-1-carbonyl}-4-fluoro-N-{phenyl[4-(propan-2-yl)phenyl]methyl}pyrrolidine-2-carboxamide;
1-[2-(1-acetylpiperidin-4-yl)propanoyl]-4-fluoro-N-{phenyl[4-(propan-2-yl)phenyl]methyl}pyrrolidine-2-carboxamide;
4-fluoro-1-[2-methyl-2-(1H-1,2,4-triazol-5-yl)propanoyl]-N-{phenyl[4-(propan-2-yl)phenyl]methyl}pyrrolidine-2-carboxamide;
4-fluoro-N-{[3-fluoro-4-(propan-2-yl)phenyl](phenyl)methyl}-1-[2-methyl-2-(1,3,4-oxadiazol-2-yl)propanoyl]pyrrolidine-2-carboxamide;
4-fluoro-N-{[3-fluoro-4-(propan-2-yl)phenyl](phenyl)methyl}-1-[2-(pyridin-4-yl)acetyl]pyrrolidine-2-carboxamide;
4-fluoro-N-{[3-fluoro-4-(propan-2-yl)phenyl](phenyl)methyl}-1-(2-{imidazo[1,2-a]pyridin-3-yl}acetyl)pyrrolidine-2-carboxamide;
4-fluoro-N-{[3-fluoro-4-(propan-2-yl)phenyl](phenyl)methyl}-1-[2-(1H-imidazol-1-yl)acetyl]pyrrolidine-2-carboxamide;
4-fluoro-N-{[3-fluoro-4-(propan-2-yl)phenyl](phenyl)methyl}-1-[2-(1,3,5-trimethyl-1H-pyrazol-4-yl)acetyl]pyrrolidine-2-carboxamide;
4-fluoro-N-{[3-fluoro-4-(propan-2-yl)phenyl](phenyl)methyl}-1-[2-(1,2-oxazol-5-yl)acetyl]pyrrolidine-2-carboxamide;
1-{2-[(dimethylcarbamoyl)amino]acetyl}-4-fluoro-N-{[3-fluoro-4-(propan-2-yl)phenyl](phenyl)methyl}pyrrolidine-2-carboxamide;
N-{[3-fluoro-4-(propan-2-yl)phenyl](phenyl)methyl}-4-[2-(1H-1,2,3-triazol-5-yl)acetyl]-4-azaspiro[2.4]heptane-5-carboxamide;
4-fluoro-N-{[3-fluoro-4-(propan-2-yl)phenyl](phenyl)methyl}-1-(2-{[1,2,4]triazolo[1,5-a]pyridin-6-yl}acetyl)pyrrolidine-2-carboxamide;
4-fluoro-N-{[3-fluoro-4-(propan-2-yl)phenyl](phenyl)methyl}-1-[2-(quinolin-6-yl)acetyl]pyrrolidine-2-carboxamide;
4-fluoro-N-{[3-fluoro-4-(propan-2-yl)phenyl](phenyl)methyl}-1-[2-(1-methyl-1H-pyrazol-4-yl)acetyl]pyrrolidine-2-carboxamide;
4-fluoro-N-{phenyl[4-(propan-2-yl)phenyl]methyl}-1-[2-(piperazin-1-yl)acetyl]pyrrolidine-2-carboxamide;
1-(1-acetyl-3-methylpiperidine-3-carbonyl)-4-fluoro-N-{phenyl[4-(propan-2-yl)phenyl]methyl}pyrrolidine-2-carboxamide;
1-(1-acetyl-4-methylazepane-4-carbonyl)-4-fluoro-N-{phenyl[4-(propan-2-yl)phenyl]methyl}pyrrolidine-2-carboxamide;
1-(1-acetylazepane-4-carbonyl)-4-fluoro-N-{phenyl[4-(propan-2-yl)phenyl]methyl}pyrrolidine-2-carboxamide;
1-(1-acetylpiperidine-3-carbonyl)-4-fluoro-N-{phenyl[4-(propan-2-yl)phenyl]methyl}pyrrolidine-2-carboxamide;
1-(4-acetylmorpholine-3-carbonyl)-4-fluoro-N-{phenyl[4-(propan-2-yl)phenyl]methyl}pyrrolidine-2-carboxamide;
1-(2-{2-acetyl-2-azaspiro[3.4]octan-5-yl}acetyl)-4-fluoro-N-{phenyl[4-(propan-2-yl)phenyl]methyl}pyrrolidine-2-carboxamide;
1-{2-acetyl-2-azaspiro[4.4]nonane-6-carbonyl}-4-fluoro-N-{phenyl[4-(propan-2-yl)phenyl]methyl}pyrrolidine-2-carboxamide;
1-{2-acetyl-2-azabicyclo[2.2.2]octane-6-carbonyl}-4-fluoro-N-{phenyl[4-(propan-2-yl)phenyl]methyl}pyrrolidine-2-carboxamide;
1-[2-(1-acetylpiperidin-3-yl)acetyl]-4-fluoro-N-{phenyl[4-(propan-2-yl)phenyl]methyl}pyrrolidine-2-carboxamide;
1-(4-acetyl-1,4-oxazepane-2-carbonyl)-4-fluoro-N-{phenyl[4-(propan-2-yl)phenyl]methyl}pyrrolidine-2-carboxamide;
1-(1-acetyl-4-methylpiperidine-3-carbonyl)-4-fluoro-N-{phenyl[4-(propan-2-yl)phenyl]methyl}pyrrolidine-2-carboxamide;
1-(4-acetyl-2-methylmorpholine-3-carbonyl)-4-fluoro-N-{phenyl[4-(propan-2-yl)phenyl]methyl}pyrrolidine-2-carboxamide;
1-{5-acetyl-hexahydro-2H-furo[2,3-c]pyrrole-3-carbonyl}-4-fluoro-N-{phenyl[4-(propan-2-yl)phenyl]methyl}pyrrolidine-2-carboxamide;
1-{3-acetyl-3-azabicyclo[3.1.0]hexane-1-carbonyl}-4-fluoro-N-{phenyl[4-(propan-2-yl)phenyl]methyl}pyrrolidine-2-carboxamide;
1-{2-acetyl-octahydrocyclopenta[c]pyrrole-4-carbonyl}-4-fluoro-N-{phenyl[4-(propan-2-yl)phenyl]methyl}pyrrolidine-2-carboxamide;
1-{8-acetyl-8-azabicyclo[3.2.1]octane-3-carbonyl}-4-fluoro-N-{phenyl[4-(propan-2-yl)phenyl]methyl}pyrrolidine-2-carboxamide;
1-(1-acetyl-3-methylazetidine-3-carbonyl)-4-fluoro-N-{phenyl[4-(propan-2-yl)phenyl]methyl}pyrrolidine-2-carboxamide;
1-{5-acetyl-hexahydro-2H-furo[2,3-c]pyrrole-2-carbonyl}-4-fluoro-N-{phenyl[4-(propan-2-yl)phenyl]methyl}pyrrolidine-2-carboxamide;
1-{3-acetyl-3-azabicyclo[3.2.1]octane-8-carbonyl}-4-fluoro-N-{phenyl[4-(propan-2-yl)phenyl]methyl}pyrrolidine-2-carboxamide;
1-(2-acetyl-octahydro-1H-isoindole-4-carbonyl)-4-fluoro-N-{phenyl[4-(propan-2-yl)phenyl]methyl}pyrrolidine-2-carboxamide;
1-{7-acetyl-7-azabicyclo[2.2.1]heptane-2-carbonyl}-4-fluoro-N-{phenyl[4-(propan-2-yl)phenyl]methyl}pyrrolidine-2-carboxamide;
1-{2-acetyl-5-oxa-2-azaspiro[3.4]octane-7-carbonyl}-4-fluoro-N-{phenyl[4-(propan-2-yl)phenyl]methyl}pyrrolidine-2-carboxamide;
1-[2-(1-acetyl-3-methylazetidin-3-yl)acetyl]-4-fluoro-N-{phenyl[4-(propan-2-yl)phenyl]methyl}pyrrolidine-2-carboxamide;
1-{2-acetyl-5-oxa-2-azaspiro[3.4]octane-6-carbonyl}-4-fluoro-N-{phenyl[4-(propan-2-yl)phenyl]methyl}pyrrolidine-2-carboxamide;
1-(1-acetyl-2-methylpiperidine-3-carbonyl)-4-fluoro-N-{phenyl[4-(propan-2-yl)phenyl]methyl}pyrrolidine-2-carboxamide;
4-fluoro-1-{3-[N-(1-methyl-1H-pyrazol-3-yl)acetamido]propanoyl}-N-{phenyl[4-(propan-2-yl)phenyl]methyl}pyrrolidine-2-carboxamide;
1-(1-acetyl-3-fluoroazetidine-3-carbonyl)-4-fluoro-N-{phenyl[4-(propan-2-yl)phenyl]methyl}pyrrolidine-2-carboxamide;
1-[2-(1-acetyl-3-methoxyazetidin-3-yl)acetyl]-4-fluoro-N-{phenyl[4-(propan-2-yl)phenyl]methyl}pyrrolidine-2-carboxamide;
1-{4-acetyl-hexahydro-2H-furo[3,2-b]pyrrole-6-carbonyl}-4-fluoro-N-{phenyl[4-(propan-2-yl)phenyl]methyl}pyrrolidine-2-carboxamide;
1-(1-acetyl-4-ethylpyrrolidine-3-carbonyl)-4-fluoro-N-{phenyl[4-(propan-2-yl)phenyl]methyl}pyrrolidine-2-carboxamide;
1-{7-acetyl-1-oxo-2,7-diazaspiro[4.4]nonane-4-carbonyl}-4-fluoro-N-{phenyl[4-(propan-2-yl)phenyl]methyl}pyrrolidine-2-carboxamide;
4-fluoro-N-{[3-fluoro-4-(propan-2-yl)phenyl](phenyl)methyl}-1-[1-(1,3,4-oxadiazol-2-yl)cyclopropanecarbonyl]pyrrolidine-2-carboxamide;
2-[4-fluoro-2-({[3-fluoro-4-(propan-2-yl)phenyl](phenyl)methyl}carbamoyl)pyrrolidin-1-yl]-2-oxoethyl N,N-dimethylcarbamate;
1-[2-(1H-1,3-benzodiazol-1-yl)acetyl]-4-fluoro-N-{[3-fluoro-4-(propan-2-yl)phenyl](phenyl)methyl}pyrrolidine-2-carboxamide;
1-[2-(1H-1,3-benzodiazol-1-yl)acetyl]-N-[(4-cyclopropyl-3-fluorophenyl)(phenyl)methyl]-4-fluoropyrrolidine-2-carboxamide;
2-[4-fluoro-2-({phenyl[5-(propan-2-yl)pyridin-2-yl]methyl}carbamoyl)pyrrolidin-1-yl]-2-oxoethyl N,N-dimethylcarbamate;
4-fluoro-N-{[3-fluoro-4-(propan-2-yl)phenyl](phenyl)methyl}-1-[2-(1-methyl-1H-1,2,3-triazol-4-yl)acetyl]pyrrolidine-2-carboxamide;
N-[(4-cyclopropyl-3-fluorophenyl)(phenyl)methyl]-4-fluoro-1-[2-(5-methyl-2,4-dioxo-1,2,3,4-tetrahydropyrimidin-1-yl)acetyl]pyrrolidine-2-carboxamide;
4-fluoro-N-{[3-fluoro-4-(propan-2-yl)phenyl](phenyl)methyl}-1-[2-(5-methyl-2,4-dioxo-1,2,3,4-tetrahydropyrimidin-1-yl)acetyl]pyrrolidine-2-carboxamide;
1-{2-[(dimethylcarbamoyl)amino]acetyl}-4-fluoro-N-{phenyl[5-(propan-2-yl)pyridin-2-yl]methyl}pyrrolidine-2-carboxamide;
N-[(4-cyclopropyl-3-fluorophenyl)(phenyl)methyl]-1-{2-[(dimethylcarbamoyl)amino]acetyl}-4-fluoropyrrolidine-2-carboxamide;
4-fluoro-1-[2-(5-methyl-2,4-dioxo-1,2,3,4-tetrahydropyrimidin-1-yl)acetyl]-N-{phenyl[5-(propan-2-yl)pyridin-2-yl]methyl}pyrrolidine-2-carboxamide;
4-fluoro-N-{phenyl[5-(propan-2-yl)pyridin-2-yl]methyl}-1-[2-(quinolin-5-yl)acetyl]pyrrolidine-2-carboxamide;
tert-butyl 4-({2-[4-fluoro-2-({[3-fluoro-4-(propan-2-yl)phenyl](phenyl)methyl}carbamoyl)pyrrolidin-1-yl]-2-oxoethyl}carbamoyl)piperazine-1-carboxylate;
N-[(4-cyclopropyl-3-fluorophenyl)(phenyl)methyl]-1-[2-(3,5-dimethyl-1H-pyrazol-4-yl)acetyl]-4-fluoropyrrolidine-2-carboxamide;
N-{2-[4-fluoro-2-({[3-fluoro-4-(propan-2-yl)phenyl](phenyl)methyl}carbamoyl)pyrrolidin-1-yl]-2-oxoethyl}piperazine-1-carboxamide;
N-[(4-cyclopropyl-3-fluorophenyl)(phenyl)methyl]-4-fluoro-1-[2-(quinolin-5-yl)acetyl]pyrrolidine-2-carboxamide;
4-fluoro-N-{phenyl[5-(propan-2-yl)pyridin-2-yl]methyl}-1-[2-(1H-1,2,3,4-tetrazol-1-yl)acetyl]pyrrolidine-2-carboxamide;
1-[2-(4-acetylpiperazin-1-yl)acetyl]-4-fluoro-N-{phenyl[4-(propan-2-yl)phenyl]methyl}pyrrolidine-2-carboxamide;
4-fluoro-1-[2-(5-methyl-2-oxo-2,3-dihydro-1,3,4-oxadiazol-3-yl)acetyl]-N-{phenyl[4-(propan-2-yl)phenyl]methyl}pyrrolidine-2-carboxamide;
1-[2-(3,5-dimethyl-2,4-dioxo-1,2,3,4-tetrahydropyrimidin-1-yl)acetyl]-4-fluoro-N-{[3-fluoro-4-(propan-2-yl)phenyl](phenyl)methyl}pyrrolidine-2-carboxamide;
4-fluoro-N-{[6-fluoro-5-(propan-2-yl)pyridin-2-yl](phenyl)methyl}-1-[2-(1H-1,2,3-triazol-5-yl)acetyl]pyrrolidine-2-carboxamide;
1-{2-[(dimethylcarbamoyl)(methyl)amino]acetyl}-4-fluoro-N-{[3-fluoro-4-(propan-2-yl)phenyl](phenyl)methyl}pyrrolidine-2-carboxamide;
4-fluoro-N-{[3-fluoro-4-(propan-2-yl)phenyl](phenyl)methyl}-1-(2-oxo-1,3-oxazolidine-5-carbonyl)pyrrolidine-2-carboxamide;
2-[4-fluoro-2-({[3-fluoro-4-(propan-2-yl)phenyl](phenyl)methyl}carbamoyl)pyrrolidin-1-yl]-2-oxoethyl piperazine-1-carboxylate;
1-[2-(3,5-dimethyl-1H-pyrazol-4-yl)acetyl]-4-fluoro-N-{phenyl[5-(propan-2-yl)pyridin-2-yl]methyl}pyrrolidine-2-carboxamide;
1-tert-butyl 4-{2-[4-fluoro-2-({[3-fluoro-4-(propan-2-yl)phenyl](phenyl)methyl}carbamoyl)pyrrolidin-1-yl]-2-oxoethyl}piperazine-1,4-dicarboxylate;
1-{2-[5-(difluoromethyl)-1,3,4-oxadiazol-2-yl]acetyl}-4-fluoro-N-{phenyl[5-(propan-2-yl)pyridin-2-yl]methyl}pyrrolidine-2-carboxamide;
1-[2-(1H-1,3-benzodiazol-1-yl)acetyl]-4-fluoro-N-{phenyl[5-(propan-2-yl)pyridin-2-yl]methyl}pyrrolidine-2-carboxamide;
1-{2-[5-(difluoromethyl)-1,3,4-oxadiazol-2-yl]acetyl}-4-fluoro-N-{[3-fluoro-4-(propan-2-yl)phenyl](phenyl)methyl}pyrrolidine-2-carboxamide;
tert-butyl N-({5-[2-(2-{[(4-cyclopropyl-3-fluorophenyl)(phenyl)methyl]carbamoyl}-4-fluoropyrrolidin-1-yl)-2-oxoethyl]-1,3,4-oxadiazol-2-yl}methyl)carbamate;
4-fluoro-N-{[3-fluoro-4-(propan-2-yl)phenyl](phenyl)methyl}-1-[2-(pyridazin-4-yl)acetyl]pyrrolidine-2-carboxamide;
4-fluoro-N-{phenyl[5-(propan-2-yl)pyridin-2-yl]methyl}-1-{2-[5-(trifluoromethyl)-2H-1,2,3,4-tetrazol-2-yl]acetyl}pyrrolidine-2-carboxamide;
4-fluoro-N-{[3-fluoro-4-(propan-2-yl)phenyl](phenyl)methyl}-5-methyl-1-[2-(1H-1,2,3-triazol-5-yl)acetyl]pyrrolidine-2-carboxamide;
4-fluoro-N-{[3-fluoro-4-(propan-2-yl)phenyl](phenyl)methyl}-1-[2-(pyridazin-3-yl)acetyl]pyrrolidine-2-carboxamide;
N-[(5-cyclopropylpyridin-2-yl)(phenyl)methyl]-4-fluoro-1-[2-(1H-1,2,3-triazol-5-yl)acetyl]pyrrolidine-2-carboxamide;
N-[(4-cyclopropyl-3-fluorophenyl)(phenyl)methyl]-4-fluoro-1-[2-(2-oxopiperazin-1-yl)acetyl]pyrrolidine-2-carboxamide;
N-[2-(2-{[(4-cyclopropyl-3-fluorophenyl)(phenyl)methyl]carbamoyl}-4-fluoropyrrolidin-1-yl)-2-oxoethyl]-4-methylpiperazine-1-carboxamide;
N-[2-(2-{[(4-cyclopropyl-3-fluorophenyl)(phenyl)methyl]carbamoyl}-4-fluoropyrrolidin-1-yl)-2-oxoethyl]-4-(2,2,2-trifluoroethyl)piperazine-1-carboxamide;
N-{2-[4-fluoro-2-({phenyl[5-(propan-2-yl)pyridin-2-yl]methyl}carbamoyl)pyrrolidin-1-yl]-2-oxoethyl}-4-(2,2,2-trifluoroethyl)piperazine-1-carboxamide;
N-[2-(2-{[(4-cyclopropyl-3-fluorophenyl)(phenyl)methyl]carbamoyl}-4-fluoropyrrolidin-1-yl)-2-oxoethyl]-4-(2-methoxyethyl)piperazine-1-carboxamide;
1-{2-[5-(aminomethyl)-1,3,4-oxadiazol-2-yl]acetyl}-N-[(4-cyclopropyl-3-fluorophenyl)(phenyl)methyl]-4-fluoropyrrolidine-2-carboxamide;
4-fluoro-N-{[3-fluoro-4-(propan-2-yl)phenyl](phenyl)methyl}-1-[2-(5-methyl-1H-1,2,3-triazol-1-yl)acetyl]pyrrolidine-2-carboxamide;
4-fluoro-N-{[3-fluoro-4-(propan-2-yl)phenyl](phenyl)methyl}-1-[2-(4-methyl-1H-1,2,3-triazol-1-yl)acetyl]pyrrolidine-2-carboxamide;
4-fluoro-N-{[3-fluoro-4-(propan-2-yl)phenyl](phenyl)methyl}-1-{2-[4-(piperazin-1-yl)-2H-1,2,3-triazol-2-yl]acetyl}pyrrolidine-2-carboxamide;
N-[2-(2-{[(4-cyclopropyl-3-fluorophenyl)(phenyl)methyl]carbamoyl}-4-fluoropyrrolidin-1-yl)-2-oxoethyl]-4-(cyclopropylmethyl)piperazine-1-carboxamide;
4-(cyclopropylmethyl)-N-{2-[4-fluoro-2-({phenyl[5-(propan-2-yl)pyridin-2-yl]methyl}carbamoyl)pyrrolidin-1-yl]-2-oxoethyl}piperazine-1-carboxamide;
N-{2-[4-fluoro-2-({phenyl[5-(propan-2-yl)pyridin-2-yl]methyl}carbamoyl)pyrrolidin-1-yl]-2-oxoethyl}-4-methylpiperazine-1-carboxamide;
4-fluoro-N-{[3-fluoro-4-(propan-2-yl)phenyl](phenyl)methyl}-1-[2-(4-methyl-1H-1,2,3-triazol-5-yl)acetyl]pyrrolidine-2-carboxamide;
4-fluoro-N-{[3-fluoro-4-(propan-2-yl)phenyl](phenyl)methyl}-1-{2-[4-(trifluoromethyl)-1H-1,2,3-triazol-1-yl]acetyl}pyrrolidine-2-carboxamide;
4-fluoro-1-[2-(2-methylquinolin-5-yl)acetyl]-N-{phenyl[5-(propan-2-yl)pyridin-2-yl]methyl}pyrrolidine-2-carboxamide;
tert-butyl N-[(5-{2-[4-fluoro-2-({phenyl[4-(propan-2-yl)phenyl]methyl}carbamoyl)pyrrolidin-1-yl]-2-oxoethyl}-1,3,4-oxadiazol-2-yl)methyl]carbamate;
1-{2-[5-(aminomethyl)-1,3,4-oxadiazol-2-yl]acetyl}-4-fluoro-N-{phenyl[4-(propan-2-yl)phenyl]methyl}pyrrolidine-2-carboxamide;
1-{2-[4-(dimethylamino)-1H-1,2,3-triazol-1-yl]acetyl}-4-fluoro-N-{[3-fluoro-4-(propan-2-yl)phenyl](phenyl)methyl}pyrrolidine-2-carboxamide;
tert-butyl 4-(5-{2-[4-fluoro-2-({[3-fluoro-4-(propan-2-yl)phenyl](phenyl)methyl}carbamoyl)pyrrolidin-1-yl]-2-oxoethyl}-1H-1,2,3-triazol-4-yl)piperazine-1-carboxylate;
4-fluoro-N-{[3-fluoro-4-(propan-2-yl)phenyl](phenyl)methyl}-1-(2-{3-oxo-2H,3H-[1,2,4]triazolo[4,3-a]pyridin-8-yl}acetyl)pyrrolidine-2-carboxamide;
4-fluoro-N-{[3-fluoro-4-(propan-2-yl)phenyl](phenyl)methyl}-1-{2-[5-(trifluoromethyl)-1H-1,2,3-triazol-1-yl]acetyl}pyrrolidine-2-carboxamide;
1-{2-[4-(dimethylamino)-2H-1,2,3-triazol-2-yl]acetyl}-4-fluoro-N-{[3-fluoro-4-(propan-2-yl)phenyl](phenyl)methyl}pyrrolidine-2-carboxamide;
1-[2-(3,5-dimethyl-2,4-dioxo-1,2,3,4-tetrahydropyrimidin-1-yl)acetyl]-4-fluoro-N-{[6-fluoro-5-(propan-2-yl)pyridin-2-yl](phenyl)methyl}pyrrolidine-2-carboxamide;
1-{2-[5-(difluoromethyl)-2H-1,2,3,4-tetrazol-2-yl]acetyl}-4-fluoro-N-{phenyl[5-(propan-2-yl)pyridin-2-yl]methyl}pyrrolidine-2-carboxamide;
1-{2-[5-(acetamidomethyl)-1,3,4-oxadiazol-2-yl]acetyl}-4-fluoro-N-{phenyl[4-(propan-2-yl)phenyl]methyl}pyrrolidine-2-carboxamide;
1-{2-[5-(aminomethyl)-1H-1,2,3-triazol-1-yl]acetyl}-4-fluoro-N-{phenyl[4-(propan-2-yl)phenyl]methyl}pyrrolidine-2-carboxamide;
1-(2-{5-[(dimethylamino)methyl]-1H-1,2,3-triazol-1-yl}acetyl)-4-fluoro-N-{phenyl[4-(propan-2-yl)phenyl]methyl}pyrrolidine-2-carboxamide;
4-fluoro-N-{[3-fluoro-4-(propan-2-yl)phenyl](phenyl)methyl}-1-{2-[4-(piperazin-1-yl)-1H-1,2,3-triazol-5-yl]acetyl}pyrrolidine-2-carboxamide;
4-fluoro-N-{[3-fluoro-4-(propan-2-yl)phenyl](phenyl)methyl}-1-{2-[4-(morpholin-4-yl)-1H-1,2,3-triazol-5-yl]acetyl}pyrrolidine-2-carboxamide;
1-(2-{5-[(dimethylamino)methyl]-1,3,4-oxadiazol-2-yl}acetyl)-4-fluoro-N-{phenyl[4-(propan-2-yl)phenyl]methyl}pyrrolidine-2-carboxamide;
4-fluoro-N-{[3-fluoro-4-(propan-2-yl)phenyl](phenyl)methyl}-1-[2-(4H-1,2,4-triazol-4-yl)acetyl]pyrrolidine-2-carboxamide;
N-[(4-cyclopropyl-3-fluorophenyl)(phenyl)methyl]-4-fluoro-1-[2-(4H-1,2,4-triazol-4-yl)acetyl]pyrrolidine-2-carboxamide;
N-[(4-cyclopropyl-3-fluorophenyl)(phenyl)methyl]-4-fluoro-1-[2-(1H-indazol-3-yl)acetyl]pyrrolidine-2-carboxamide;
4-fluoro-N-{[3-fluoro-4-(propan-2-yl)phenyl](phenyl)methyl}-1-{2-[5-(trifluoromethyl)-1H-1,2,3,4-tetrazol-1-yl]acetyl}pyrrolidine-2-carboxamide;
1-[2-(1H-1,2,3-benzotriazol-1-yl)acetyl]-N-[(4-cyclopropyl-3-fluorophenyl)(phenyl)methyl]-4-fluoropyrrolidine-2-carboxamide;
N-[(4-cyclopropyl-3-fluorophenyl)(phenyl)methyl]-4-fluoro-1-[2-(3-methyl-2-oxo-2,3-dihydro-1H-1,3-benzodiazol-1-yl)acetyl]pyrrolidine-2-carboxamide;
N-[(4-cyclopropyl-3-fluorophenyl)(phenyl)methyl]-4-fluoro-1-[2-(3-methyl-2,4-dioxo-1,2,3,4-tetrahydropyrimidin-1-yl)acetyl]pyrrolidine-2-carboxamide;
N-[(4-cyclopropyl-3-fluorophenyl)(phenyl)methyl]-4-fluoro-1-[2-(2-oxo-2,3-dihydro-1H-1,3-benzodiazol-1-yl)acetyl]pyrrolidine-2-carboxamide;
N-[(4-cyclopropyl-3-fluorophenyl)(phenyl)methyl]-4-fluoro-1-[2-(1-oxo-2,3-dihydro-1H-isoindol-2-yl)acetyl]pyrrolidine-2-carboxamide;
N-[2-(2-{[(4-cyclopropyl-3-fluorophenyl)(phenyl)methyl]carbamoyl}-4-fluoropyrrolidin-1-yl)-2-oxoethyl]morpholine-4-carboxamide;
N-[(4-cyclopropyl-3-fluorophenyl)(phenyl)methyl]-4-fluoro-1-[2-(1-methyl-1H-indol-3-yl)acetyl]pyrrolidine-2-carboxamide;
N-[(4-cyclopropyl-3-fluorophenyl)(phenyl)methyl]-4-fluoro-1-[2-(1-methyl-1H-indol-2-yl)acetyl]pyrrolidine-2-carboxamide;
2-(2-{[(4-cyclopropyl-3-fluorophenyl)(phenyl)methyl]carbamoyl}-4-fluoropyrrolidin-1-yl)-2-oxoethyl azetidine-1-carboxylate;
N-[(4-cyclopropyl-3-fluorophenyl)(phenyl)methyl]-4-fluoro-1-[2-(1-methyl-1H-indazol-3-yl)acetyl]pyrrolidine-2-carboxamide;
N-[(4-cyclopropyl-3-fluorophenyl)(phenyl)methyl]-4-fluoro-1-[2-(2-oxo-2,3-dihydro-1H-indol-1-yl)acetyl]pyrrolidine-2-carboxamide;
N-[(4-cyclopropyl-3-fluorophenyl)(phenyl)methyl]-4-fluoro-1-[2-(2-methyl-1H-1,3-benzodiazol-1-yl)acetyl]pyrrolidine-2-carboxamide;
1-{2-[5-(acetamidomethyl)-1H-1,2,3-triazol-1-yl]acetyl}-4-fluoro-N-{phenyl[4-(propan-2-yl)phenyl]methyl}pyrrolidine-2-carboxamide;
4-fluoro-1-[2-(5-oxo-4,5-dihydro-1H-1,2,4-triazol-3-yl)acetyl]-N-{phenyl[5-(propan-2-yl)pyridin-2-yl]methyl}pyrrolidine-2-carboxamide;
N-[(4-cyclopropyl-3-fluorophenyl)(phenyl)methyl]-4-fluoro-1-[2-(5-oxo-4,5-dihydro-1H-1,2,4-triazol-3-yl)acetyl]pyrrolidine-2-carboxamide;
N-[(4-cyclopropyl-3-fluorophenyl)(phenyl)methyl]-4-fluoro-1-[2-(5-oxo-4,5-dihydro-1,2,4-oxadiazol-3-yl)acetyl]pyrrolidine-2-carboxamide;
N-[(4-cyclopropyl-3-fluorophenyl)(phenyl)methyl]-4-fluoro-1-[2-(1-methyl-2-oxo-2,3-dihydro-1H-indol-3-yl)acetyl]pyrrolidine-2-carboxamide;
N-[(4-cyclopropyl-3-fluorophenyl)(phenyl)methyl]-4-fluoro-1-[2-(4-methyl-1H-imidazol-1-yl)acetyl]pyrrolidine-2-carboxamide;
1-{2-[5-(difluoromethyl)-1H-1,2,3,4-tetrazol-1-yl]acetyl}-4-fluoro-N-{phenyl[5-(propan-2-yl)pyridin-2-yl]methyl}pyrrolidine-2-carboxamide;
2-(2-{[(4-cyclopropyl-3-fluorophenyl)(phenyl)methyl]carbamoyl}-4-fluoropyrrolidin-1-yl)-2-oxoethyl 4-(cyclopropylmethyl)piperazine-1-carboxylate;
2-(2-{[(4-cyclopropyl-3-fluorophenyl)(phenyl)methyl]carbamoyl}-4-fluoropyrrolidin-1-yl)-2-oxoethyl 4-methylpiperazine-1-carboxylate;
N-[(4-cyclopropyl-3-fluorophenyl)(phenyl)methyl]-4-fluoro-1-[2-(2-oxo-2,3-dihydro-1H-indol-3-yl)acetyl]pyrrolidine-2-carboxamide;
N-[(4-cyclopropyl-3-fluorophenyl)(phenyl)methyl]-4-fluoro-1-(2-{imidazo[1,2-a]pyridin-3-yl}acetyl)pyrrolidine-2-carboxamide;
N-[(4-cyclopropyl-3-fluorophenyl)(phenyl)methyl]-4-fluoro-1-(2-{[1,2,4]triazolo[1,5-a]pyridin-6-yl}acetyl)pyrrolidine-2-carboxamide;
4-fluoro-N-{[3-fluoro-4-(propan-2-yl)phenyl](phenyl)methyl}-1-{2-[4-(trifluoromethyl)-1H-1,2,3-triazol-5-yl]acetyl}pyrrolidine-2-carboxamide;
N-[(4-cyclopropyl-3-fluorophenyl)(phenyl)methyl]-4-fluoro-1-{2-[(pyrazin-2-yl)amino]acetyl}pyrrolidine-2-carboxamide;
4-fluoro-N-{[3-fluoro-4-(propan-2-yl)phenyl](phenyl)methyl}-1-{2-[4-(piperazin-1-yl)-1H-1,2,3-triazol-1-yl]acetyl}pyrrolidine-2-carboxamide;
N-[(4-cyclopropyl-3-fluorophenyl)(phenyl)methyl]-1-{2-[(2-ethyl-2H-1,2,3-triazol-4-yl)amino]acetyl}-4-fluoropyrrolidine-2-carboxamide;
4-fluoro-N-{[6-fluoro-5-(propan-2-yl)pyridin-2-yl](phenyl)methyl}-1-[2-(5-methyl-2,4-dioxo-1,2,3,4-tetrahydropyrimidin-1-yl)acetyl]pyrrolidine-2-carboxamide;
N-[(4-cyclopropyl-3-fluorophenyl)(phenyl)methyl]-1-{2-[(1-ethyl-1H-1,2,3-triazol-4-yl)amino]acetyl}-4-fluoropyrrolidine-2-carboxamide;
N-[(4-cyclopropyl-3-fluorophenyl)(phenyl)methyl]-4-fluoro-1-[2-(2-methylquinolin-6-yl)acetyl]pyrrolidine-2-carboxamide;
2-(2-{[(4-cyclopropyl-3-fluorophenyl)(phenyl)methyl]carbamoyl}-4-fluoropyrrolidin-1-yl)-2-oxoethyl 4-(2-methoxyethyl)piperazine-1-carboxylate;
N-[(4-cyclopropyl-3-fluorophenyl)(phenyl)methyl]-4-fluoro-1-{2-[2-oxo-4-(2,2,2-trifluoroethyl)piperazin-1-yl]acetyl}pyrrolidine-2-carboxamide;
N-[(4-cyclopropyl-3-fluorophenyl)(phenyl)methyl]-4-fluoro-1-{2-[methyl(2-methylpyrimidin-4-yl)amino]acetyl}pyrrolidine-2-carboxamide;
N-[(4-cyclopropyl-3-fluorophenyl)(phenyl)methyl]-4-fluoro-1-[2-(2-methylquinolin-5-yl)acetyl]pyrrolidine-2-carboxamide;
N-[(4-cyclopropyl-3-fluorophenyl)(phenyl)methyl]-4-fluoro-1-{2-[(2-methylpyrimidin-4-yl)amino]acetyl}pyrrolidine-2-carboxamide;
4-fluoro-1-[2-(4-methyl-5-oxo-4,5-dihydro-1,2,4-oxadiazol-3-yl)acetyl]-N-{phenyl[5-(propan-2-yl)pyridin-2-yl]methyl}pyrrolidine-2-carboxamide;
N-[(4-cyclopropyl-3-fluorophenyl)(phenyl)methyl]-4-fluoro-1-{2-[methyl(pyrazin-2-yl)amino]acetyl}pyrrolidine-2-carboxamide;
4-fluoro-1-(2-{3-oxo-2H,3H-[1,2,4]triazolo[4,3-a]pyridin-8-yl}acetyl)-N-{phenyl[5-(propan-2-yl)pyridin-2-yl]methyl}pyrrolidine-2-carboxamide;
2-(2-{[(4-cyclopropyl-3-fluorophenyl)(phenyl)methyl]carbamoyl}-4-fluoropyrrolidin-1-yl)-2-oxoethyl 4-(2,2,2-trifluoroethyl)piperazine-1-carboxylate;
N-[(5-cyclopropyl-6-fluoropyridin-2-yl)(phenyl)methyl]-4-fluoro-1-[2-(4H-1,2,4-triazol-4-yl)acetyl]pyrrolidine-2-carboxamide;
4-fluoro-N-{[6-fluoro-5-(propan-2-yl)pyridin-2-yl](phenyl)methyl}-1-[2-(4H-1,2,4-triazol-4-yl)acetyl]pyrrolidine-2-carboxamide;
1-[2-(1H-1,3-benzodiazol-1-yl)acetyl]-N-[(5-cyclopropyl-6-fluoropyridin-2-yl)(phenyl)methyl]-4-fluoropyrrolidine-2-carboxamide;
1-[2-(1H-1,3-benzodiazol-1-yl)acetyl]-4-fluoro-N-{[6-fluoro-5-(propan-2-yl)pyridin-2-yl](phenyl)methyl}pyrrolidine-2-carboxamide;
N-[(4-cyclopropyl-3-fluorophenyl)(phenyl)methyl]-1-{2-[4-(dimethylamino)-2H-1,2,3-triazol-2-yl]acetyl}-4-fluoropyrrolidine-2-carboxamide;
N-[(4-cyclopropyl-3-fluorophenyl)(phenyl)methyl]-1-{2-[4-(dimethylamino)-1H-1,2,3-triazol-1-yl]acetyl}-4-fluoropyrrolidine-2-carboxamide;
1-{2-[4-(dimethylamino)-1H-1,2,3-triazol-1-yl]acetyl}-4-fluoro-N-{phenyl[5-(propan-2-yl)pyridin-2-yl]methyl}pyrrolidine-2-carboxamide;
N-[(4-cyclopropyl-3-fluorophenyl)(phenyl)methyl]-4-fluoro-1-(2-{3-oxo-2H,3H-[1,2,4]triazolo[4,3-a]pyridin-8-yl}acetyl)pyrrolidine-2-carboxamide;
4-fluoro-N-{[6-fluoro-5-(propan-2-yl)pyridin-2-yl](phenyl)methyl}-1-(2-{3-oxo-2H,3H-[1,2,4]triazolo[4,3-a]pyridin-8-yl}acetyl)pyrrolidine-2-carboxamide;
4-fluoro-N-{[6-fluoro-5-(propan-2-yl)pyridin-2-yl](phenyl)methyl}-1-[2-(2-methylquinolin-5-yl)acetyl]pyrrolidine-2-carboxamide;
tert-butyl 2-[2-(2-{[(4-cyclopropyl-3-fluorophenyl)(phenyl)methyl]carbamoyl}-4-fluoropyrrolidin-1-yl)-2-oxoethyl]-1H-indole-1-carboxylate;
1-{2-[4-(dimethylamino)-2H-1,2,3-triazol-2-yl]acetyl}-4-fluoro-N-{phenyl[5-(propan-2-yl)pyridin-2-yl]methyl}pyrrolidine-2-carboxamide;
N-[(4-cyclopropyl-3-fluorophenyl)(phenyl)methyl]-4-fluoro-1-[2-(1H-indol-2-yl)acetyl]pyrrolidine-2-carboxamide;
1-[2-(carbamoylamino)acetyl]-4-fluoro-N-{[6-fluoro-5-(propan-2-yl)pyridin-2-yl](phenyl)methyl}pyrrolidine-2-carboxamide;
1-{2-[4-(dimethylamino)-1H-1,2,3-triazol-1-yl]acetyl}-4-fluoro-N-{[6-fluoro-5-(propan-2-yl)pyridin-2-yl](phenyl)methyl}pyrrolidine-2-carboxamide;
N-[(4-cyclopropyl-3-fluorophenyl)(phenyl)methyl]-1-{2-[(2-ethyl-2H-1,2,3-triazol-4-yl)(methyl)amino]acetyl}-4-fluoropyrrolidine-2-carboxamide;
N-[(4-cyclopropyl-3-fluorophenyl)(phenyl)methyl]-1-{2-[(1-ethyl-1H-1,2,3-triazol-4-yl)(methyl)amino]acetyl}-4-fluoropyrrolidine-2-carboxamide;
N-[(5-cyclopropyl-6-fluoropyridin-2-yl)(phenyl)methyl]-4-fluoro-1-[2-(5-methyl-2,4-dioxo-1,2,3,4-tetrahydropyrimidin-1-yl)acetyl]pyrrolidine-2-carboxamide;
N-[(5-cyclopropyl-6-fluoropyridin-2-yl)(phenyl)methyl]-1-{2-[4-(dimethylamino)-1H-1,2,3-triazol-1-yl]acetyl}-4-fluoropyrrolidine-2-carboxamide;
1-{2-[4-(dimethylamino)-2H-1,2,3-triazol-2-yl]acetyl}-4-fluoro-N-{[6-fluoro-5-(propan-2-yl)pyridin-2-yl](phenyl)methyl}pyrrolidine-2-carboxamide;
N-[(5-cyclopropyl-6-fluoropyridin-2-yl)(phenyl)methyl]-1-{2-[5-(dimethylamino)-1H-1,2,3-triazol-1-yl]acetyl}-4-fluoropyrrolidine-2-carboxamide;
4-fluoro-N-{phenyl[5-(propan-2-yl)pyridin-2-yl]methyl}-1-[2-(4H-1,2,4-triazol-4-yl)acetyl]pyrrolidine-2-carboxamide;
N-[(4-cyclopropyl-3-fluorophenyl)(phenyl)methyl]-4-fluoro-1-[3-(5-oxo-4,5-dihydro-1H-1,2,4-triazol-3-yl)propanoyl]pyrrolidine-2-carboxamide;
N-[(4-cyclopropyl-3-fluorophenyl)(phenyl)methyl]-4-fluoro-1-[2-(4-methyl-5-oxo-4,5-dihydro-1,2,4-oxadiazol-3-yl)acetyl]pyrrolidine-2-carboxamide;
4-fluoro-1-[2-(5-oxo-4,5-dihydro-1,2,4-oxadiazol-3-yl)acetyl]-N-{phenyl[5-(propan-2-yl)pyridin-2-yl]methyl}pyrrolidine-2-carboxamide;
1-{2-[(dimethylcarbamoyl)amino]acetyl}-4-fluoro-N-{[6-fluoro-5-(propan-2-yl)pyridin-2-yl](phenyl)methyl}pyrrolidine-2-carboxamide;
4-fluoro-1-{2-[(methylcarbamoyl)amino]acetyl}-N-{phenyl[5-(propan-2-yl)pyridin-2-yl]methyl}pyrrolidine-2-carboxamide;
4-fluoro-N-{[6-fluoro-5-(propan-2-yl)pyridin-2-yl](phenyl)methyl}-1-{2-[(2-methylpyrimidin-4-yl)amino]acetyl}pyrrolidine-2-carboxamide;
4-fluoro-N-{[6-fluoro-5-(propan-2-yl)pyridin-2-yl](phenyl)methyl}-1-{2-[(methylcarbamoyl)amino]acetyl}pyrrolidine-2-carboxamide;
4-fluoro-N-{[6-fluoro-5-(propan-2-yl)pyridin-2-yl](phenyl)methyl}-1-[2-(4-methyl-5-oxo-4,5-dihydro-1,3,4-oxadiazol-2-yl)acetyl]pyrrolidine-2-carboxamide;
1-{2-[(azetidine-1-carbonyl)amino]acetyl}-4-fluoro-N-{phenyl[5-(propan-2-yl)pyridin-2-yl]methyl}pyrrolidine-2-carboxamide;
1-{2-[(azetidine-1-carbonyl)amino]acetyl}-4-fluoro-N-{[6-fluoro-5-(propan-2-yl)pyridin-2-yl](phenyl)methyl}pyrrolidine-2-carboxamide;
4-fluoro-1-{2-[(2-methylpyrimidin-4-yl)amino]acetyl}-N-{phenyl[5-(propan-2-yl)pyridin-2-yl]methyl}pyrrolidine-2-carboxamide;
N-[(5-cyclopropyl-6-fluoropyridin-2-yl)(phenyl)methyl]-1-{2-[(dimethylcarbamoyl)amino]acetyl}-4-fluoropyrrolidine-2-carboxamide;
1-[2-(carbamoylamino)acetyl]-4-fluoro-N-{phenyl[5-(propan-2-yl)pyridin-2-yl]methyl}pyrrolidine-2-carboxamide;
4-fluoro-1-[2-(4-methyl-5-oxo-4,5-dihydro-1,3,4-oxadiazol-2-yl)acetyl]-N-{phenyl[5-(propan-2-yl)pyridin-2-yl]methyl}pyrrolidine-2-carboxamide;
4-fluoro-1-[3-(5-oxo-4,5-dihydro-1H-1,2,4-triazol-3-yl)propanoyl]-N-{phenyl[5-(propan-2-yl)pyridin-2-yl]methyl}pyrrolidine-2-carboxamide;
1-[2-(carbamoylamino)acetyl]-N-[(5-cyclopropyl-6-fluoropyridin-2-yl)(phenyl)methyl]-4-fluoropyrrolidine-2-carboxamide;
1-{2-[(azetidine-1-carbonyl)amino]acetyl}-N-[(5-cyclopropyl-6-fluoropyridin-2-yl)(phenyl)methyl]-4-fluoropyrrolidine-2-carboxamide;
N-[(5-cyclopropyl-6-fluoropyridin-2-yl)(phenyl)methyl]-4-fluoro-1-{2-[(methylcarbamoyl)amino]acetyl}pyrrolidine-2-carboxamide;
4-fluoro-1-[2-(1-methyl-5-oxo-4,5-dihydro-1H-1,2,4-triazol-3-yl)acetyl]-N-{phenyl[5-(propan-2-yl)pyridin-2-yl]methyl}pyrrolidine-2-carboxamide;
4-fluoro-N-{[6-fluoro-5-(propan-2-yl)pyridin-2-yl](phenyl)methyl}-1-[2-(4-methyl-5-oxo-4,5-dihydro-1,2,4-oxadiazol-3-yl)acetyl]pyrrolidine-2-carboxamide;
N-[(5-cyclopropyl-6-fluoropyridin-2-yl)(phenyl)methyl]-4-fluoro-1-{2-[(2-methylpyrimidin-4-yl)amino]acetyl}pyrrolidine-2-carboxamide;
N-[(5-cyclopropyl-6-fluoropyridin-2-yl)(phenyl)methyl]-4-fluoro-1-[2-(4-methyl-5-oxo-4,5-dihydro-1,3,4-oxadiazol-2-yl)acetyl]pyrrolidine-2-carboxamide;
N-[(5-cyclopropyl-6-fluoropyridin-2-yl)(phenyl)methyl]-4-fluoro-1-[2-(1,3-oxazol-2-yl)acetyl]pyrrolidine-2-carboxamide;
4-fluoro-1-[2-(1,3-oxazol-2-yl)acetyl]-N-{phenyl[5-(propan-2-yl)pyridin-2-yl]methyl}pyrrolidine-2-carboxamide;
N-[(5-cyclopropyl-6-fluoropyridin-2-yl)(phenyl)methyl]-1-{2-[5-(difluoromethyl)-1H-1,2,3,4-tetrazol-1-yl]acetyl}-4-fluoropyrrolidine-2-carboxamide;
1-{2-[5-(difluoromethyl)-1H-1,2,3,4-tetrazol-1-yl]acetyl}-4-fluoro-N-{[6-fluoro-5-(propan-2-yl)pyridin-2-yl](phenyl)methyl}pyrrolidine-2-carboxamide;
N-[(5-cyclopropyl-6-fluoropyridin-2-yl)(phenyl)methyl]-4-fluoro-1-[2-(1-methyl-5-oxo-4,5-dihydro-1H-1,2,4-triazol-3-yl)acetyl]pyrrolidine-2-carboxamide;
4-fluoro-N-{[6-fluoro-5-(propan-2-yl)pyridin-2-yl](phenyl)methyl}-1-[2-(1-methyl-5-oxo-4,5-dihydro-1H-1,2,4-triazol-3-yl)acetyl]pyrrolidine-2-carboxamide;
4-fluoro-N-{[6-fluoro-5-(propan-2-yl)pyridin-2-yl](phenyl)methyl}-1-{2-[4-(trifluoromethyl)-1H-1,2,3-triazol-5-yl]acetyl}pyrrolidine-2-carboxamide;
4-fluoro-N-{phenyl[5-(propan-2-yl)pyridin-2-yl]methyl}-1-{2-[4-(trifluoromethyl)-1H-1,2,3-triazol-5-yl]acetyl}pyrrolidine-2-carboxamide;
1-{2-[(dimethylcarbamoyl)amino]propanoyl}-4-fluoro-N-{[6-fluoro-5-(propan-2-yl)pyridin-2-yl](phenyl)methyl}pyrrolidine-2-carboxamide;
N-[(4-cyclopropyl-3-fluorophenyl)(phenyl)methyl]-1-{2-[(3,3-difluoroazetidine-1-carbonyl)amino]acetyl}-4-fluoropyrrolidine-2-carboxamide;
N-[(4-cyclopropyl-3-fluorophenyl)(phenyl)methyl]-4-fluoro-1-(2-{[3-(trifluoromethyl)azetidine-1-carbonyl]amino}acetyl)pyrrolidine-2-carboxamide;
N-[(5-cyclopropyl-6-fluoropyridin-2-yl)(phenyl)methyl]-4-fluoro-1-{2-[4-(trifluoromethyl)-1H-1,2,3-triazol-5-yl]acetyl}pyrrolidine-2-carboxamide;
N-[(5-cyclopropyl-6-fluoropyridin-2-yl)(phenyl)methyl]-1-{2-[(dimethylcarbamoyl)amino]propanoyl}-4-fluoropyrrolidine-2-carboxamide;
4-fluoro-1-[2-(5-methyl-1,3-oxazol-2-yl)acetyl]-N-{phenyl[5-(propan-2-yl)pyridin-2-yl]methyl}pyrrolidine-2-carboxamide;
1-{2-[(dimethylcarbamoyl)amino]propanoyl}-4-fluoro-N-{phenyl[5-(propan-2-yl)pyridin-2-yl]methyl}pyrrolidine-2-carboxamide;
4-fluoro-N-{[6-fluoro-5-(propan-2-yl)pyridin-2-yl](phenyl)methyl}-1-[2-(5-methyl-1,3-oxazol-2-yl)acetyl]pyrrolidine-2-carboxamide;
N-[(5-cyclopropyl-6-fluoropyridin-2-yl)(phenyl)methyl]-4-fluoro-1-[2-(5-methyl-1,3-oxazol-2-yl)acetyl]pyrrolidine-2-carboxamide;
4-fluoro-N-{[6-fluoro-5-(propan-2-yl)pyridin-2-yl](phenyl)methyl}-1-[2-(1,3-oxazol-2-yl)acetyl]pyrrolidine-2-carboxamide;
4-fluoro-1-[2-(5-oxo-4,5-dihydro-1H-1,2,4-triazol-4-yl)acetyl]-N-{phenyl[5-(propan-2-yl)pyridin-2-yl]methyl}pyrrolidine-2-carboxamide;
4-fluoro-1-(3-{3-oxo-2H,3H-[1,2,4]triazolo[4,3-a]pyridin-2-yl}propanoyl)-N-{phenyl[5-(propan-2-yl)pyridin-2-yl]methyl}pyrrolidine-2-carboxamide;
1-{2-[(3,3-difluoroazetidine-1-carbonyl)amino]acetyl}-4-fluoro-N-{phenyl[5-(propan-2-yl)pyridin-2-yl]methyl}pyrrolidine-2-carboxamide;
4-fluoro-N-{phenyl[5-(propan-2-yl)pyridin-2-yl]methyl}-1-(2-{[3-(trifluoromethyl)azetidine-1-carbonyl]amino}acetyl)pyrrolidine-2-carboxamide;
4-fluoro-N-{[6-fluoro-5-(propan-2-yl)pyridin-2-yl](phenyl)methyl}-1-[2-(4-methyl-5-oxo-4,5-dihydro-1H-1,2,4-triazol-3-yl)acetyl]pyrrolidine-2-carboxamide;
1-{2-[(3,3-difluoroazetidine-1-carbonyl)amino]acetyl}-4-fluoro-N-{[6-fluoro-5-(propan-2-yl)pyridin-2-yl](phenyl)methyl}pyrrolidine-2-carboxamide;
4-fluoro-1-[2-(4-methyl-5-oxo-4,5-dihydro-1H-1,2,4-triazol-3-yl)acetyl]-N-{phenyl[5-(propan-2-yl)pyridin-2-yl]methyl}pyrrolidine-2-carboxamide;
N-[(5-cyclopropyl-6-fluoropyridin-2-yl)(phenyl)methyl]-1-{2-[(3,3-difluoroazetidine-1-carbonyl)amino]acetyl}-4-fluoropyrrolidine-2-carboxamide;
N-{phenyl[5-(propan-2-yl)pyridin-2-yl]methyl}-3-[2-(1H-1,2,3-triazol-5-yl)acetyl]-3-azabicyclo[3.1.0]hexane-2-carboxamide;
5-methyl-N-{phenyl[5-(propan-2-yl)pyridin-2-yl]methyl}-1-[2-(1H-1,2,3-triazol-5-yl)acetyl]pyrrolidine-2-carboxamide;
N-[(5-cyclopropyl-6-fluoropyridin-2-yl)(phenyl)methyl]-4-fluoro-1-[2-(4-methyl-1,3-oxazol-2-yl)acetyl]pyrrolidine-2-carboxamide;
N-[(5-cyclopropyl-6-fluoropyridin-2-yl)(phenyl)methyl]-4-fluoro-1-(2-{3-oxo-2H,3H-[1,2,4]triazolo[4,3-a]pyridin-8-yl}acetyl)pyrrolidine-2-carboxamide;
4-fluoro-1-[2-(4-methyl-1,3-oxazol-2-yl)acetyl]-N-{phenyl[5-(propan-2-yl)pyridin-2-yl]methyl}pyrrolidine-2-carboxamide;
N-[(5-cyclopropyl-6-fluoropyridin-2-yl)(phenyl)methyl]-4-fluoro-1-[2-(4-methyl-5-oxo-4,5-dihydro-1H-1,2,4-triazol-3-yl)acetyl]pyrrolidine-2-carboxamide;
4-fluoro-N-{[6-fluoro-5-(propan-2-yl)pyridin-2-yl](phenyl)methyl}-1-[2-(4-methyl-1,3-oxazol-2-yl)acetyl]pyrrolidine-2-carboxamide;
4-fluoro-N-{phenyl[5-(propan-2-yl)pyridin-2-yl]methyl}-1-[2-(1H-pyrazol-1-yl)acetyl]pyrrolidine-2-carboxamide;
1-{2-[4-(3,3-difluoroazetidin-1-yl)-2H-1,2,3-triazol-2-yl]acetyl}-4-fluoro-N-{[6-fluoro-5-(propan-2-yl)pyridin-2-yl](phenyl)methyl}pyrrolidine-2-carboxamide;
1-{2-[4-(diethylamino)-2H-1,2,3-triazol-2-yl]acetyl}-4-fluoro-N-{[6-fluoro-5-(propan-2-yl)pyridin-2-yl](phenyl)methyl}pyrrolidine-2-carboxamide;
N-{[6-fluoro-5-(propan-2-yl)pyridin-2-yl](phenyl)methyl}-5-methyl-1-[2-(1H-1,2,3-triazol-5-yl)acetyl]pyrrolidine-2-carboxamide;
1-{2-[4-(3,3-difluoroazetidin-1-yl)-2H-1,2,3-triazol-2-yl]acetyl}-4-fluoro-N-{phenyl[5-(propan-2-yl)pyridin-2-yl]methyl}pyrrolidine-2-carboxamide;
N-[(5-cyclopropyl-6-fluoropyridin-2-yl)(phenyl)methyl]-4-fluoro-1-{2-[(1,3-oxazol-2-yl)amino]acetyl}pyrrolidine-2-carboxamide;
N-{[6-fluoro-5-(propan-2-yl)pyridin-2-yl](phenyl)methyl}-3-[2-(1H-1,2,3-triazol-5-yl)acetyl]-3-azabicyclo[3.1.0]hexane-2-carboxamide;
N-[(5-cyclopropyl-6-fluoropyridin-2-yl)(phenyl)methyl]-1-{2-[4-(3,3-difluoroazetidin-1-yl)-2H-1,2,3-triazol-2-yl]acetyl}-4-fluoropyrrolidine-2-carboxamide;
1-{2-[4-(diethylamino)-2H-1,2,3-triazol-2-yl]acetyl}-4-fluoro-N-{phenyl[5-(propan-2-yl)pyridin-2-yl]methyl}pyrrolidine-2-carboxamide;
N-[(5-cyclopropyl-6-fluoropyridin-2-yl)(phenyl)methyl]-1-{2-[4-(diethylamino)-2H-1,2,3-triazol-2-yl]acetyl}-4-fluoropyrrolidine-2-carboxamide;
N-[(5-cyclopropyl-6-fluoropyridin-2-yl)(phenyl)methyl]-5-methyl-1-[2-(1H-1,2,3-triazol-5-yl)acetyl]pyrrolidine-2-carboxamide;
1-{2-[4-(azetidin-1-yl)-2H-1,2,3-triazol-2-yl]acetyl}-4-fluoro-N-{phenyl[5-(propan-2-yl)pyridin-2-yl]methyl}pyrrolidine-2-carboxamide;
1-{2-[4-(azetidin-1-yl)-2H-1,2,3-triazol-2-yl]acetyl}-N-[(5-cyclopropyl-6-fluoropyridin-2-yl)(phenyl)methyl]-4-fluoropyrrolidine-2-carboxamide;
1-{2-[4-(azetidin-1-yl)-2H-1,2,3-triazol-2-yl]acetyl}-4-fluoro-N-{[6-fluoro-5-(propan-2-yl)pyridin-2-yl](phenyl)methyl}pyrrolidine-2-carboxamide;
4-fluoro-1-[2-(5-methyl-2-oxo-2,3-dihydro-1,3,4-oxadiazol-3-yl)acetyl]-N-{phenyl[5-(propan-2-yl)pyridin-2-yl]methyl}pyrrolidine-2-carboxamide;
N-[(5-cyclopropyl-6-fluoropyridin-2-yl)(phenyl)methyl]-4-fluoro-1-[2-(5-methyl-2-oxo-2,3-dihydro-1,3,4-oxadiazol-3-yl)acetyl]pyrrolidine-2-carboxamide;
4-fluoro-N-{[6-fluoro-5-(propan-2-yl)pyridin-2-yl](phenyl)methyl}-1-[2-(5-methyl-2-oxo-2,3-dihydro-1,3,4-oxadiazol-3-yl)acetyl]pyrrolidine-2-carboxamide;
N-[(5-cyclopropyl-6-fluoropyridin-2-yl)(phenyl)methyl]-3-[2-(1H-1,2,3-triazol-5-yl)acetyl]-3-azabicyclo[3.1.0]hexane-2-carboxamide;
N-{[5-(3,3-difluorocyclobutyl)-6-fluoropyridin-2-yl](phenyl)methyl}-4-fluoro-1-[2-(5-methyl-2-oxo-2,3-dihydro-1,3,4-oxadiazol-3-yl)acetyl]pyrrolidine-2-carboxamide;
1-{2-[(azetidine-1-carbonyl)amino]acetyl}-N-{[5-(3,3-difluorocyclobutyl)-6-fluoropyridin-2-yl](phenyl)methyl}-4-fluoropyrrolidine-2-carboxamide;
1-[2-(1H-1,3-benzodiazol-1-yl)acetyl]-N-{[5-(3,3-difluorocyclobutyl)-6-fluoropyridin-2-yl](phenyl)methyl}-4-fluoropyrrolidine-2-carboxamide;
N-{[5-(3,3-difluorocyclobutyl)-6-fluoropyridin-2-yl](phenyl)methyl}-4-fluoro-1-[2-(1,3-oxazol-2-yl)acetyl]pyrrolidine-2-carboxamide;
N-{[5-(3,3-difluorocyclobutyl)-6-fluoropyridin-2-yl](phenyl)methyl}-4-fluoro-1-[2-(5-methyl-2,4-dioxo-1,2,3,4-tetrahydropyrimidin-1-yl)acetyl]pyrrolidine-2-carboxamide;
N-{[5-(3,3-difluorocyclobutyl)-6-fluoropyridin-2-yl](phenyl)methyl}-1-{2-[(dimethylcarbamoyl)amino]acetyl}-4-fluoropyrrolidine-2-carboxamide;
N-{[5-(3,3-difluorocyclobutyl)-6-fluoropyridin-2-yl](phenyl)methyl}-4-fluoro-1-[2-(1H-1,2,3-triazol-5-yl)acetyl]pyrrolidine-2-carboxamide;
N-{[5-(3,3-difluorocyclobutyl)-6-fluoropyridin-2-yl](phenyl)methyl}-4-fluoro-1-[2-(5-methyl-1,3-oxazol-2-yl)acetyl]pyrrolidine-2-carboxamide;
N-{[5-(3,3-difluorocyclobutyl)-6-fluoropyridin-2-yl](phenyl)methyl}-4-fluoro-1-{2-[4-(trifluoromethyl)-1H-1,2,3-triazol-5-yl]acetyl}pyrrolidine-2-carboxamide;
N-{[5-(3,3-difluorocyclobutyl)-6-fluoropyridin-2-yl](phenyl)methyl}-4-fluoro-1-[2-(4-methyl-1,3-oxazol-2-yl)acetyl]pyrrolidine-2-carboxamide;
1-{2-[(3,3-difluoroazetidine-1-carbonyl)amino]acetyl}-N-{[5-(3,3-difluorocyclobutyl)-6-fluoropyridin-2-yl](phenyl)methyl}-4-fluoropyrrolidine-2-carboxamide;
N-{[5-(3,3-difluorocyclobutyl)-6-fluoropyridin-2-yl](phenyl)methyl}-1-{2-[5-(difluoromethyl)-1H-1,2,3,4-tetrazol-1-yl]acetyl}-4-fluoropyrrolidine-2-carboxamide;
N-{[5-(3,3-difluorocyclobutyl)-6-fluoropyridin-2-yl](phenyl)methyl}-1-[2-(2,4-dioxo-1,2,3,4-tetrahydropyrimidin-1-yl)acetyl]-4-fluoropyrrolidine-2-carboxamide;
N-{[5-(3,3-difluorocyclobutyl)-6-fluoropyridin-2-yl](phenyl)methyl}-1-[2-(3-ethyl-2,4-dioxo-1,2,3,4-tetrahydropyrimidin-1-yl)acetyl]-4-fluoropyrrolidine-2-carboxamide;
N-{[5-(3,3-difluorocyclobutyl)-6-fluoropyridin-2-yl](phenyl)methyl}-4-fluoro-1-[2-(6-oxo-1,6-dihydropyridin-3-yl)acetyl]pyrrolidine-2-carboxamide;
N-{[5-(3,3-difluorocyclobutyl)-6-fluoropyridin-2-yl](phenyl)methyl}-4-fluoro-1-[2-(2-oxo-1,2-dihydropyridin-3-yl)acetyl]pyrrolidine-2-carboxamide;
N-{[5-(3,3-difluorocyclobutyl)-6-fluoropyridin-2-yl](phenyl)methyl}-1-[2-(3-ethyl-5-methyl-2,4-dioxo-1,2,3,4-tetrahydropyrimidin-1-yl)acetyl]-4-fluoropyrrolidine-2-carboxamide;
N-{[5-(3,3-difluorocyclobutyl)-6-fluoropyridin-2-yl](phenyl)methyl}-1-[2-(1-ethyl-6-oxo-1,6-dihydropyridin-3-yl)acetyl]-4-fluoropyrrolidine-2-carboxamide;
N-{[5-(3,3-difluorocyclobutyl)-6-fluoropyridin-2-yl](phenyl)methyl}-4-fluoro-1-[2-(5-methyl-6-oxo-1,6-dihydropyridin-3-yl)acetyl]pyrrolidine-2-carboxamide;
N-{[5-(3,3-difluorocyclobutyl)-6-fluoropyridin-2-yl](phenyl)methyl}-1-[2-(1-ethyl-5-methyl-6-oxo-1,6-dihydropyridin-3-yl)acetyl]-4-fluoropyrrolidine-2-carboxamide;
N-{[5-(3,3-difluorocyclobutyl)-6-fluoropyridin-2-yl](phenyl)methyl}-1-[2-(6-ethoxy-5-methylpyridin-3-yl)acetyl]-4-fluoropyrrolidine-2-carboxamide;
N-{[5-(3,3-difluorocyclobutyl)-6-fluoropyridin-2-yl](phenyl)methyl}-1-[2-(1-ethyl-5-methyl-2-oxo-1,2-dihydropyridin-3-yl)acetyl]-4-fluoropyrrolidine-2-carboxamide;
N-{[5-(3,3-difluorocyclobutyl)-6-fluoropyridin-2-yl](phenyl)methyl}-1-[2-(1-ethyl-2-oxo-1,2-dihydropyridin-3-yl)acetyl]-4-fluoropyrrolidine-2-carboxamide;
N-{[5-(3,3-difluorocyclobutyl)-6-fluoropyridin-2-yl](phenyl)methyl}-1-[2-(4-ethyl-5-oxo-4,5-dihydropyrazin-2-yl)acetyl]-4-fluoropyrrolidine-2-carboxamide;
N-{[5-(3,3-difluorocyclobutyl)-6-fluoropyridin-2-yl](phenyl)methyl}-4-fluoro-1-{2-[4-(trifluoromethyl)-1,3-oxazol-2-yl]acetyl}pyrrolidine-2-carboxamide;
N-{[5-(3,3-difluorocyclobutyl)-6-fluoropyridin-2-yl](phenyl)methyl}-4-fluoro-1-[2-(3-oxo-3,4-dihydropyrazin-2-yl)acetyl]pyrrolidine-2-carboxamide;
N-{[5-(3,3-difluorocyclobutyl)-6-fluoropyridin-2-yl](phenyl)methyl}-4-fluoro-1-[2-(5-oxo-4,5-dihydropyrazin-2-yl)acetyl]pyrrolidine-2-carboxamide;
N-{[5-(3,3-difluorocyclobutyl)-6-fluoropyridin-2-yl](phenyl)methyl}-1-[2-(4-ethyl-3-oxo-3,4-dihydropyrazin-2-yl)acetyl]-4-fluoropyrrolidine-2-carboxamide;
N-{[5-(3,3-difluorocyclobutyl)pyridin-2-yl](phenyl)methyl}-1-{2-[(dimethylcarbamoyl)amino]acetyl}-4-fluoropyrrolidine-2-carboxamide;
1-{2-[(azetidine-1-carbonyl)amino]acetyl}-N-{[5-(3,3-difluorocyclobutyl)pyridin-2-yl](phenyl)methyl}-4-fluoropyrrolidine-2-carboxamide;
N-{[5-(3,3-difluorocyclobutyl)pyridin-2-yl](phenyl)methyl}-4-fluoro-1-[2-(1H-1,2,3-triazol-5-yl)acetyl]pyrrolidine-2-carboxamide;
N-{[5-(3,3-difluorocyclobutyl)pyridin-2-yl](phenyl)methyl}-4-fluoro-1-[2-(5-methyl-2,4-dioxo-1,2,3,4-tetrahydropyrimidin-1-yl)acetyl]pyrrolidine-2-carboxamide;
N-{[5-(3,3-difluorocyclobutyl)pyridin-2-yl](phenyl)methyl}-4-fluoro-1-[2-(5-methyl-1,3-oxazol-2-yl)acetyl]pyrrolidine-2-carboxamide;
1-[2-(1H-1,3-benzodiazol-1-yl)acetyl]-N-{[5-(3,3-difluorocyclobutyl)pyridin-2-yl](phenyl)methyl}-4-fluoropyrrolidine-2-carboxamide;
1-{2-[(3,3-difluoroazetidine-1-carbonyl)amino]acetyl}-N-{[5-(3,3-difluorocyclobutyl)pyridin-2-yl](phenyl)methyl}-4-fluoropyrrolidine-2-carboxamide;
N-{[5-(3,3-difluorocyclobutyl)pyridin-2-yl](phenyl)methyl}-4-fluoro-1-[2-(1,3-oxazol-2-yl)acetyl]pyrrolidine-2-carboxamide;
N-{[5-(3,3-difluorocyclobutyl)pyridin-2-yl](phenyl)methyl}-4-fluoro-1-[2-(4-methyl-1,3-oxazol-2-yl)acetyl]pyrrolidine-2-carboxamide;
N-{[5-(3,3-difluorocyclobutyl)pyridin-2-yl](phenyl)methyl}-4-fluoro-1-[2-(5-methyl-2-oxo-2,3-dihydro-1,3,4-oxadiazol-3-yl)acetyl]pyrrolidine-2-carboxamide;
N-{[5-(3,3-difluorocyclobutyl)pyridin-2-yl](phenyl)methyl}-4-fluoro-1-{2-[4-(trifluoromethyl)-1H-1,2,3-triazol-5-yl]acetyl}pyrrolidine-2-carboxamide;
N-{[5-(3,3-difluorocyclobutyl)pyridin-2-yl](phenyl)methyl}-4-fluoro-1-{2-[4-(trifluoromethyl)-1,3-oxazol-2-yl]acetyl}pyrrolidine-2-carboxamide;
1-{2-[(azetidine-1-carbonyl)amino]acetyl}-N-{[4-(3,3-difluorocyclobutyl)-3-fluorophenyl](phenyl)methyl}-4-fluoropyrrolidine-2-carboxamide;
N-{[4-(3,3-difluorocyclobutyl)-3-fluorophenyl](phenyl)methyl}-1-{2-[(dimethylcarbamoyl)amino]acetyl}-4-fluoropyrrolidine-2-carboxamide;
1-[2-(1H-1,3-benzodiazol-1-yl)acetyl]-N-{[4-(3,3-difluorocyclobutyl)-3-fluorophenyl](phenyl)methyl}-4-fluoropyrrolidine-2-carboxamide;
N-{[4-(3,3-difluorocyclobutyl)-3-fluorophenyl](phenyl)methyl}-4-fluoro-1-[2-(5-methyl-1,3-oxazol-2-yl)acetyl]pyrrolidine-2-carboxamide;
N-{[4-(3,3-difluorocyclobutyl)-3-fluorophenyl](phenyl)methyl}-4-fluoro-1-[2-(5-methyl-2,4-dioxo-1,2,3,4-tetrahydropyrimidin-1-yl)acetyl]pyrrolidine-2-carboxamide;
N-{[4-(3,3-difluorocyclobutyl)-3-fluorophenyl](phenyl)methyl}-4-fluoro-1-[2-(1,3-oxazol-2-yl)acetyl]pyrrolidine-2-carboxamide;
N-{[4-(3,3-difluorocyclobutyl)-3-fluorophenyl](phenyl)methyl}-4-fluoro-1-{2-[4-(trifluoromethyl)-1H-1,2,3-triazol-5-yl]acetyl}pyrrolidine-2-carboxamide;
N-{[4-(3,3-difluorocyclobutyl)-3-fluorophenyl](phenyl)methyl}-4-fluoro-1-[2-(4-methyl-1,3-oxazol-2-yl)acetyl]pyrrolidine-2-carboxamide;
N-{[4-(3,3-difluorocyclobutyl)-3-fluorophenyl](phenyl)methyl}-4-fluoro-1-{2-[4-(trifluoromethyl)-1,3-oxazol-2-yl]acetyl}pyrrolidine-2-carboxamide;
4-fluoro-N-{[3-fluoro-4-(1-methylcyclopropyl)phenyl](phenyl)methyl}-1-[2-(5-methyl-2,4-dioxo-1,2,3,4-tetrahydropyrimidin-1-yl)acetyl]pyrrolidine-2-carboxamide;
4-fluoro-N-{[3-fluoro-4-(1-methylcyclopropyl)phenyl](phenyl)methyl}-1-[2-(1H-1,2,3-triazol-5-yl)acetyl]pyrrolidine-2-carboxamide;
4-fluoro-N-{[3-fluoro-4-(1-methylcyclopropyl)phenyl](pyridin-3-yl)methyl}-1-[2-(1H-1,2,3-triazol-5-yl)acetyl]pyrrolidine-2-carboxamide;
2-[4-fluoro-2-({[3-fluoro-4-(1-methylcyclopropyl)phenyl](phenyl)methyl}carbamoyl)pyrrolidin-1-yl]-2-oxoethyl azetidine-1-carboxylate;
4-fluoro-N-{[3-fluoro-4-(1-methylcyclopropyl)phenyl](phenyl)methyl}-1-[2-(1-methyl-1H-indol-2-yl)acetyl]pyrrolidine-2-carboxamide;
4-fluoro-N-{[3-fluoro-4-(1-methylcyclopropyl)phenyl](phenyl)methyl}-1-[2-(2-oxopiperazin-1-yl)acetyl]pyrrolidine-2-carboxamide;
4-fluoro-N-{[3-fluoro-4-(1-methylcyclopropyl)phenyl](phenyl)methyl}-1-[2-(1-methyl-1H-indol-3-yl)acetyl]pyrrolidine-2-carboxamide;
4-fluoro-N-{[3-fluoro-4-(1-methylcyclopropyl)phenyl](phenyl)methyl}-1-[2-(1-methyl-1H-indazol-3-yl)acetyl]pyrrolidine-2-carboxamide;
N-{2-[4-fluoro-2-({[3-fluoro-4-(1-methylcyclopropyl)phenyl](phenyl)methyl}carbamoyl)pyrrolidin-1-yl]-2-oxoethyl}morpholine-4-carboxamide;
4-fluoro-N-{[3-fluoro-4-(1-methylcyclopropyl)phenyl](phenyl)methyl}-1-[2-(2-methyl-TH-1,3-benzodiazol-1-yl)acetyl]pyrrolidine-2-carboxamide;
1-[2-(1H-1,3-benzodiazol-1-yl)propanoyl]-4-fluoro-N-{[3-fluoro-4-(1-methylcyclopropyl)phenyl](phenyl)methyl}pyrrolidine-2-carboxamide;
4-fluoro-N-{[3-fluoro-4-(1-methylcyclopropyl)phenyl](phenyl)methyl}-1-[2-(3-methyl-2-oxo-2,3-dihydro-1H-1,3-benzodiazol-1-yl)acetyl]pyrrolidine-2-carboxamide;
4-fluoro-N-{[3-fluoro-4-(1-methylcyclopropyl)phenyl](phenyl)methyl}-1-[2-(3-methyl-2,4-dioxo-1,2,3,4-tetrahydropyrimidin-1-yl)acetyl]pyrrolidine-2-carboxamide;
4-fluoro-N-{[3-fluoro-4-(1-methylcyclopropyl)phenyl](phenyl)methyl}-1-[2-(2-oxo-2,3-dihydro-1H-1,3-benzodiazol-1-yl)acetyl]pyrrolidine-2-carboxamide;
4-fluoro-N-{[3-fluoro-4-(1-methylcyclopropyl)phenyl](phenyl)methyl}-1-[2-(1H-indazol-3-yl)acetyl]pyrrolidine-2-carboxamide;
1-[2-(2,5-dioxopiperazin-1-yl)acetyl]-4-fluoro-N-{[3-fluoro-4-(1-methylcyclopropyl)phenyl](phenyl)methyl}pyrrolidine-2-carboxamide;
4-fluoro-N-{[3-fluoro-4-(1-methylcyclopropyl)phenyl](phenyl)methyl}-1-[2-(1-oxo-2,3-dihydro-1H-isoindol-2-yl)acetyl]pyrrolidine-2-carboxamide;
1-[2-(1H-1,2,3-benzotriazol-1-yl)acetyl]-4-fluoro-N-{[3-fluoro-4-(1-methylcyclopropyl)phenyl](phenyl)methyl}pyrrolidine-2-carboxamide;
4-fluoro-N-{[3-fluoro-4-(1-methylcyclopropyl)phenyl](phenyl)methyl}-1-[2-(2-oxo-2,3-dihydro-1H-indol-1-yl)acetyl]pyrrolidine-2-carboxamide;
4-fluoro-N-{[3-fluoro-4-(1-methylcyclopropyl)phenyl](phenyl)methyl}-1-[2-(1-methyl-2-oxo-2,3-dihydro-1H-indol-3-yl)acetyl]pyrrolidine-2-carboxamide;
2,2,2-trifluoroethyl 4-{2-[4-fluoro-2-({[3-fluoro-4-(1-methylcyclopropyl)phenyl](phenyl)methyl}carbamoyl)pyrrolidin-1-yl]-2-oxoethyl}-3-oxopiperazine-1-carboxylate;
2-[4-fluoro-2-({[3-fluoro-4-(1-methylcyclopropyl)phenyl](phenyl)methyl}carbamoyl)pyrrolidin-1-yl]-2-oxoethyl 4-(2-methoxyethyl)piperazine-1-carboxylate;
4-fluoro-N-{[3-fluoro-4-(1-methylcyclopropyl)phenyl](phenyl)methyl}-1-{2-[2-oxo-4-(2,2,2-trifluoroethyl)piperazin-1-yl]acetyl}pyrrolidine-2-carboxamide;
4-fluoro-N-{[3-fluoro-4-(1-methylcyclopropyl)phenyl](phenyl)methyl}-1-[2-(2-oxo-2,3-dihydro-1H-indol-3-yl)acetyl]pyrrolidine-2-carboxamide;
4-fluoro-N-{[3-fluoro-4-(1-methylcyclopropyl)phenyl](phenyl)methyl}-1-[2-(4H-1,2,4-triazol-4-yl)acetyl]pyrrolidine-2-carboxamide;
2-[4-fluoro-2-({[3-fluoro-4-(1-methylcyclopropyl)phenyl](phenyl)methyl}carbamoyl)pyrrolidin-1-yl]-2-oxoethyl 4-methylpiperazine-1-carboxylate;
2-[4-fluoro-2-({[3-fluoro-4-(1-methylcyclopropyl)phenyl](phenyl)methyl}carbamoyl)pyrrolidin-1-yl]-2-oxoethyl 4-(cyclopropylmethyl)piperazine-1-carboxylate;
2-[4-fluoro-2-({[3-fluoro-4-(1-methylcyclopropyl)phenyl](phenyl)methyl}carbamoyl)pyrrolidin-1-yl]-2-oxoethyl 4-(2,2,2-trifluoroethyl)piperazine-1-carboxylate;
1-[2-(1H-1,3-benzodiazol-1-yl)propanoyl]-4-fluoro-N-{[6-fluoro-5-(1-methylcyclopropyl)pyridin-2-yl](phenyl)methyl}pyrrolidine-2-carboxamide;
4-fluoro-N-{[6-fluoro-5-(1-methylcyclopropyl)pyridin-2-yl](phenyl)methyl}-1-[2-(1H-indazol-3-yl)acetyl]pyrrolidine-2-carboxamide;
N-{2-[4-fluoro-2-({[6-fluoro-5-(1-methylcyclopropyl)pyridin-2-yl](phenyl)methyl}carbamoyl)pyrrolidin-1-yl]-2-oxoethyl}morpholine-4-carboxamide;
4-fluoro-N-{[6-fluoro-5-(1-methylcyclopropyl)pyridin-2-yl](phenyl)methyl}-1-[2-(1-methyl-1H-indol-2-yl)acetyl]pyrrolidine-2-carboxamide;
4-fluoro-N-{[6-fluoro-5-(1-methylcyclopropyl)pyridin-2-yl](phenyl)methyl}-1-[2-(1-methyl-1H-indol-3-yl)acetyl]pyrrolidine-2-carboxamide;
4-fluoro-N-{[6-fluoro-5-(1-methylcyclopropyl)pyridin-2-yl](phenyl)methyl}-1-[2-(2-methyl-1H-1,3-benzodiazol-1-yl)acetyl]pyrrolidine-2-carboxamide;
1-[2-(1H-1,2,3-benzotriazol-1-yl)acetyl]-4-fluoro-N-{[6-fluoro-5-(1-methylcyclopropyl)pyridin-2-yl](phenyl)methyl}pyrrolidine-2-carboxamide;
1-[2-(1H-1,3-benzodiazol-1-yl)acetyl]-4-fluoro-N-{[6-fluoro-5-(1-methylcyclopropyl)pyridin-2-yl](phenyl)methyl}pyrrolidine-2-carboxamide;
4-fluoro-N-{[6-fluoro-5-(1-methylcyclopropyl)pyridin-2-yl](phenyl)methyl}-1-[2-(1-methyl-1H-indazol-3-yl)acetyl]pyrrolidine-2-carboxamide;
4-fluoro-N-{[6-fluoro-5-(1-methylcyclopropyl)pyridin-2-yl](phenyl)methyl}-1-[2-(1-methyl-2-oxo-2,3-dihydro-1H-indol-3-yl)acetyl]pyrrolidine-2-carboxamide;
4-fluoro-N-{[6-fluoro-5-(1-methylcyclopropyl)pyridin-2-yl](phenyl)methyl}-1-[2-(1H-indol-2-yl)acetyl]pyrrolidine-2-carboxamide;
4-fluoro-N-{[6-fluoro-5-(1-methylcyclopropyl)pyridin-2-yl](phenyl)methyl}-1-[2-(2-oxo-2,3-dihydro-1H-1,3-benzodiazol-1-yl)acetyl]pyrrolidine-2-carboxamide;
4-fluoro-N-{[6-fluoro-5-(1-methylcyclopropyl)pyridin-2-yl](phenyl)methyl}-1-[2-(3-methyl-2-oxo-2,3-dihydro-1H-1,3-benzodiazol-1-yl)acetyl]pyrrolidine-2-carboxamide;
tert-butyl 2-{2-[4-fluoro-2-({[6-fluoro-5-(1-methylcyclopropyl)pyridin-2-yl](phenyl)methyl}carbamoyl)pyrrolidin-1-yl]-2-oxoethyl}-1H-indole-1-carboxylate;
1-{2-[5-(difluoromethyl)-1,3,4-oxadiazol-2-yl]acetyl}-4-fluoro-N-{[4-methyl-5-(propan-2-yl)pyridin-2-yl](phenyl)methyl}pyrrolidine-2-carboxamide;
1-acetyl-N-[(5-cyclobutylpyridin-2-yl)(phenyl)methyl]-4-fluoropyrrolidine-2-carboxamide;
4-fluoro-N-{[4-methyl-5-(propan-2-yl)pyridin-2-yl](phenyl)methyl}-1-[2-(1H-1,2,3-triazol-5-yl)acetyl]pyrrolidine-2-carboxamide;
4-fluoro-N-{[4-fluoro-5-(propan-2-yl)pyridin-2-yl](phenyl)methyl}-1-[2-(1H-1,2,3-triazol-5-yl)acetyl]pyrrolidine-2-carboxamide;
N-{[4-(difluoromethyl)-5-(propan-2-yl)pyridin-2-yl](phenyl)methyl}-4-fluoro-1-[2-(1H-1,2,3-triazol-5-yl)acetyl]pyrrolidine-2-carboxamide;
4-fluoro-N-{[6-methyl-5-(propan-2-yl)pyridin-2-yl](phenyl)methyl}-1-[2-(1H-1,2,3-triazol-5-yl)acetyl]pyrrolidine-2-carboxamide;
4-fluoro-N-{phenyl[5-(propan-2-yl)-4-(trifluoromethyl)pyridin-2-yl]methyl}-1-[2-(1H-1,2,3-triazol-5-yl)acetyl]pyrrolidine-2-carboxamide;
1-{2-[5-(difluoromethyl)-1,3,4-oxadiazol-2-yl]acetyl}-4-fluoro-N-{[6-methyl-5-(propan-2-yl)pyridin-2-yl](phenyl)methyl}pyrrolidine-2-carboxamide;
N-[(5-cyclobutylpyridin-2-yl)(phenyl)methyl]-4-fluoro-1-[2-(1H-1,2,3-triazol-5-yl)acetyl]pyrrolidine-2-carboxamide;
N-[(5-tert-butylpyridin-2-yl)(phenyl)methyl]-4-fluoro-1-[2-(1H-1,2,3-triazol-5-yl)acetyl]pyrrolidine-2-carboxamide;
4-fluoro-N-{[6-methoxy-5-(propan-2-yl)pyridin-2-yl](phenyl)methyl}-1-[2-(1H-1,2,3-triazol-5-yl)acetyl]pyrrolidine-2-carboxamide;
N-[(2-aminopyridin-3-yl)[3-fluoro-4-(propan-2-yl)phenyl]methyl]-4-fluoro-1-[2-(1H-1,2,3-triazol-5-yl)acetyl]pyrrolidine-2-carboxamide;
N-[(4-cyclopropyl-3-fluorophenyl)(1H-pyrazol-5-yl)methyl]-1-(2-acetamidoacetyl)pyrrolidine-2-carboxamide;
4-fluoro-N-{[3-fluoro-4-(propan-2-yl)phenyl](5-fluoropyridin-2-yl)methyl}-1-[2-(1H-1,2,3-triazol-5-yl)acetyl]pyrrolidine-2-carboxamide;
4-fluoro-N-{[3-fluoro-4-(propan-2-yl)phenyl](5-fluoropyridin-3-yl)methyl}-1-[2-(1H-1,2,3-triazol-5-yl)acetyl]pyrrolidine-2-carboxamide;
N-[(2-aminopyridin-4-yl)[3-fluoro-4-(propan-2-yl)phenyl]methyl]-4-fluoro-1-[2-(1H-1,2,3-triazol-5-yl)acetyl]pyrrolidine-2-carboxamide;
4-fluoro-N-{[3-fluoro-4-(propan-2-yl)phenyl](3-fluoropyridin-4-yl)methyl}-1-[2-(1H-1,2,3-triazol-5-yl)acetyl]pyrrolidine-2-carboxamide;
N-[(6-aminopyridin-3-yl)[3-fluoro-4-(propan-2-yl)phenyl]methyl]-4-fluoro-1-[2-(1H-1,2,3-triazol-5-yl)acetyl]pyrrolidine-2-carboxamide;
N-[(6-aminopyridin-2-yl)[3-fluoro-4-(propan-2-yl)phenyl]methyl]-4-fluoro-1-[2-(1H-1,2,3-triazol-5-yl)acetyl]pyrrolidine-2-carboxamide;
N-[(2-aminopyridin-3-yl)[3-methyl-4-(propan-2-yl)phenyl]methyl]-1-{2-[(dimethylcarbamoyl)amino]acetyl}-4-fluoropyrrolidine-2-carboxamide;
4-fluoro-N-{[3-fluoro-4-(propan-2-yl)phenyl](1H-pyrazol-5-yl)methyl}-1-[2-(1H-1,2,3-triazol-5-yl)acetyl]pyrrolidine-2-carboxamide;
N-[(2-aminopyridin-3-yl)[3-methyl-4-(propan-2-yl)phenyl]methyl]-4-fluoro-1-[2-(1H-1,2,3-triazol-5-yl)acetyl]pyrrolidine-2-carboxamide;
N-[(2-aminopyridin-3-yl)[3-methyl-4-(propan-2-yl)phenyl]methyl]-4-fluoro-1-[2-(1,3,5-trimethyl-1H-pyrazol-4-yl)acetyl]pyrrolidine-2-carboxamide;
4-fluoro-N-{[3-fluoro-4-(1-methylcyclopropyl)phenyl](2-methoxypyridin-3-yl)methyl}-1-[2-(1H-1,2,3-triazol-5-yl)acetyl]pyrrolidine-2-carboxamide;
4-fluoro-N-{[3-fluoro-4-(1-methylcyclopropyl)phenyl](2-methylpyridin-3-yl)methyl}-1-[2-(1H-1,2,3-triazol-5-yl)acetyl]pyrrolidine-2-carboxamide;
4-fluoro-N-{[6-fluoro-5-(propan-2-yl)pyridin-2-yl]({imidazo[1,5-a]pyridin-3-yl})methyl}-1-[2-(1H-1,2,3-triazol-5-yl)acetyl]pyrrolidine-2-carboxamide;
4-fluoro-N-{[6-fluoro-5-(propan-2-yl)pyridin-2-yl]({imidazo[1,5-a]pyridin-1-yl})methyl}-1-[2-(1H-1,2,3-triazol-5-yl)acetyl]pyrrolidine-2-carboxamide;
4-fluoro-N-{[6-fluoro-5-(propan-2-yl)pyridin-2-yl](1H-pyrazol-5-yl)methyl}-1-[2-(1,3-oxazol-2-yl)acetyl]pyrrolidine-2-carboxamide;
4-fluoro-N-{[6-fluoro-5-(propan-2-yl)pyridin-2-yl]({imidazo[1,5-a]pyridin-7-yl})methyl}-1-[2-(1H-1,2,3-triazol-5-yl)acetyl]pyrrolidine-2-carboxamide;
4-fluoro-N-{[6-fluoro-5-(propan-2-yl)pyridin-2-yl](1H-indazol-6-yl)methyl}-1-[2-(1H-1,2,3-triazol-5-yl)acetyl]pyrrolidine-2-carboxamide;
1-acetyl-4-fluoro-N-{[6-fluoro-5-(propan-2-yl)pyridin-2-yl](1H-indazol-6-yl)methyl}pyrrolidine-2-carboxamide;
1-acetyl-4-fluoro-N-{[6-fluoro-5-(propan-2-yl)pyridin-2-yl](1-methyl-TH-indazol-6-yl)methyl}pyrrolidine-2-carboxamide;
1-acetyl-4-fluoro-N-{[6-fluoro-5-(propan-2-yl)pyridin-2-yl](2-methyl-2H-indazol-6-yl)methyl}pyrrolidine-2-carboxamide;
1-acetyl-N-[(5-cyclopropyl-6-fluoropyridin-2-yl)(1H-indazol-6-yl)methyl]-4-fluoropyrrolidine-2-carboxamide;
methyl N-({3-[({4-fluoro-1-[2-(1H-1,2,3-triazol-5-yl)acetyl]pyrrolidin-2-yl}formamido)[6-fluoro-5-(propan-2-yl)pyridin-2-yl]methyl]phenyl}methyl)carbamate;
1-acetyl-N-[(2-methoxyphenyl)[4-(propan-2-yl)phenyl]methyl]pyrrolidine-2-carboxamide;
1-acetyl-N-[(2-methylphenyl)[4-(propan-2-yl)phenyl]methyl]pyrrolidine-2-carboxamide;
1-acetyl-N-[(2-methylphenyl)[5-(propan-2-yl)pyridin-2-yl]methyl]pyrrolidine-2-carboxamide;
N-[(4-cyclopropyl-3-fluorophenyl)(2-oxo-2,3-dihydro-1,3-benzoxazol-7-yl)methyl]-1-(2-acetamidoacetyl)pyrrolidine-2-carboxamide;
N-[(2-oxo-2,3-dihydro-1H-1,3-benzodiazol-4-yl)[4-(propan-2-yl)phenyl]methyl]-1-[2-(1H-1,2,3-triazol-5-yl)acetyl]pyrrolidine-2-carboxamide;
4-fluoro-N-{[3-fluoro-4-(propan-2-yl)phenyl](3-fluorophenyl)methyl}-1-[2-(1H-1,2,3-triazol-5-yl)acetyl]pyrrolidine-2-carboxamide;
4-fluoro-N-{[3-fluoro-4-(propan-2-yl)phenyl](4-fluorophenyl)methyl}-1-[2-(1H-1,2,3-triazol-5-yl)acetyl]pyrrolidine-2-carboxamide;
N-[(4-cyclopropyl-3-fluorophenyl)(2-oxo-2,3-dihydro-TH-1,3-benzodiazol-4-yl)methyl]-1-[2-(1H-1,2,3-triazol-5-yl)acetyl]pyrrolidine-2-carboxamide;
N-[(3-acetamidophenyl)[4-(propan-2-yl)phenyl]methyl]-4-fluoro-1-[2-(1-methyl-1H-1,2,3-triazol-4-yl)acetyl]pyrrolidine-2-carboxamide;
N-[(4-cyclopropyl-3-fluorophenyl)(1-methyl-2-oxo-2,3-dihydro-TH-1,3-benzodiazol-4-yl)methyl]-4-fluoro-1-[2-(1H-1,2,3-triazol-5-yl)acetyl]pyrrolidine-2-carboxamide;
4-fluoro-N-{[6-fluoro-5-(propan-2-yl)pyridin-2-yl](3-methoxyphenyl)methyl}-1-[2-(1H-1,2,3-triazol-5-yl)acetyl]pyrrolidine-2-carboxamide;
N-{cyclopropyl[3-fluoro-4-(propan-2-yl)phenyl]methyl}-4-fluoro-1-[2-(1H-1,2,3-triazol-5-yl)acetyl]pyrrolidine-2-carboxamide;
N-{2-cyclopropyl-1-[3-fluoro-4-(propan-2-yl)phenyl]ethyl}-4-fluoro-1-[2-(1H-1,2,3-triazol-5-yl)acetyl]pyrrolidine-2-carboxamide;
1-acetyl-4-fluoro-N-{[6-fluoro-5-(propan-2-yl)pyridin-2-yl](3-methoxyphenyl)methyl}pyrrolidine-2-carboxamide;
N-{[3-(acetamidomethyl)phenyl][6-fluoro-5-(propan-2-yl)pyridin-2-yl]methyl}-4-fluoro-1-[2-(1H-1,2,3-triazol-5-yl)acetyl]pyrrolidine-2-carboxamide;
4-fluoro-N-{[6-fluoro-5-(propan-2-yl)pyridin-2-yl](3-{[(methylcarbamoyl)amino]methyl}phenyl)methyl}-1-[2-(1H-1,2,3-triazol-5-yl)acetyl]pyrrolidine-2-carboxamide;
N-[(5-cyclopropyl-6-fluoropyridin-2-yl)(2-fluorophenyl)methyl]-1-{2-[5-(difluoromethyl)-1H-1,2,3,4-tetrazol-1-yl]acetyl}-4-fluoropyrrolidine-2-carboxamide;
N-[(5-cyclopropyl-6-fluoropyridin-2-yl)(4-fluorophenyl)methyl]-1-{2-[5-(difluoromethyl)-1H-1,2,3,4-tetrazol-1-yl]acetyl}-4-fluoropyrrolidine-2-carboxamide;
N-{[3,5-difluoro-4-(propan-2-yl)phenyl](phenyl)methyl}-4-fluoro-1-[(1H-1,2,3-triazol-5-yl)methanesulfonyl]pyrrolidine-2-carboxamide;
1-acetyl-N-[(4-cyclobutyl-3-fluorophenyl)(phenyl)methyl]-4-fluoropyrrolidine-2-carboxamide;
N-{[3,5-difluoro-4-(propan-2-yl)phenyl](phenyl)methyl}-4-fluoro-1-[2-(1,3-oxazol-2-yl)acetyl]pyrrolidine-2-carboxamide;
N-[(4-cyclopropyl-3,5-difluorophenyl)(phenyl)methyl]-4-fluoro-1-[2-(1,3-oxazol-2-yl)acetyl]pyrrolidine-2-carboxamide;
4-fluoro-N-{[2-methyl-4-(propan-2-yl)phenyl](phenyl)methyl}-1-[2-(1,3-oxazol-2-yl)acetyl]pyrrolidine-2-carboxamide;
N-[(3-chloro-4-cyclopropylphenyl)(phenyl)methyl]-4-fluoro-1-[2-(1H-1,2,3-triazol-5-yl)acetyl]pyrrolidine-2-carboxamide;
N-[(4-cyclopropyl-3-methylphenyl)(phenyl)methyl]-4-fluoro-1-[2-(1H-1,2,3-triazol-5-yl)acetyl]pyrrolidine-2-carboxamide;
N-{[3,5-difluoro-4-(propan-2-yl)phenyl](phenyl)methyl}-4-fluoro-1-[2-(1H-1,2,3-triazol-5-yl)acetyl]pyrrolidine-2-carboxamide;
N-{[3,5-difluoro-4-(propan-2-yl)phenyl](phenyl)methyl}-4-fluoro-1-[2-(1H-1,2,3-triazol-1-yl)acetyl]pyrrolidine-2-carboxamide;
N-{[3,5-difluoro-4-(propan-2-yl)phenyl](phenyl)methyl}-4-fluoro-1-[2-(1H-1,2,3,4-tetrazol-1-yl)acetyl]pyrrolidine-2-carboxamide;
4-fluoro-N-{[3-methyl-4-(propan-2-yl)phenyl](phenyl)methyl}-1-[2-(1H-1,2,3-triazol-5-yl)acetyl]pyrrolidine-2-carboxamide;
N-{[3-chloro-4-(propan-2-yl)phenyl](phenyl)methyl}-4-fluoro-1-[2-(1H-1,2,3-triazol-5-yl)acetyl]pyrrolidine-2-carboxamide;
N-[(4-cyclobutyl-3-fluorophenyl)(phenyl)methyl]-4-fluoro-1-[2-(1H-1,2,3-triazol-5-yl)acetyl]pyrrolidine-2-carboxamide;
N-[(4-cyclobutyl-3-fluorophenyl)(phenyl)methyl]-4-fluoro-1-[2-(1H-1,2,3,4-tetrazol-1-yl)acetyl]pyrrolidine-2-carboxamide;
N-{[4-(butan-2-yl)-3-fluorophenyl](phenyl)methyl}-4-fluoro-1-[2-(1H-1,2,3-triazol-5-yl)acetyl]pyrrolidine-2-carboxamide;
4-fluoro-N-{[3-fluoro-5-methyl-4-(propan-2-yl)phenyl](phenyl)methyl}-1-[2-(1H-1,2,3-triazol-5-yl)acetyl]pyrrolidine-2-carboxamide;
N-{[3-(difluoromethyl)-4-(propan-2-yl)phenyl](phenyl)methyl}-4-fluoro-1-[2-(1H-1,2,3-triazol-5-yl)acetyl]pyrrolidine-2-carboxamide;
4-fluoro-N-{[3-fluoro-4-(1-methylcyclobutyl)phenyl](phenyl)methyl}-1-[2-(1H-1,2,3-triazol-5-yl)acetyl]pyrrolidine-2-carboxamide;
N-[(4-cyclopropyl-3-fluoro-5-methylphenyl)(phenyl)methyl]-4-fluoro-1-[2-(5-methyl-2H-1,2,3,4-tetrazol-2-yl)acetyl]pyrrolidine-2-carboxamide;
4-fluoro-N-{phenyl[4-(propan-2-yl)-3-(trifluoromethyl)phenyl]methyl}-1-[2-(1H-1,2,3-triazol-5-yl)acetyl]pyrrolidine-2-carboxamide;
N-[(4-tert-butyl-3-fluorophenyl)(phenyl)methyl]-4-fluoro-1-[2-(1H-1,2,3-triazol-5-yl)acetyl]pyrrolidine-2-carboxamide;
4-fluoro-N-{[6-fluoro-5-(propan-2-yl)pyridin-2-yl][3-(1H-pyrazol-5-yl)phenyl]methyl}-1-[2-(1H-1,2,3-triazol-5-yl)acetyl]pyrrolidine-2-carboxamide;
1-acetyl-4-fluoro-N-{[6-fluoro-5-(propan-2-yl)pyridin-2-yl][3-(4H-1,2,4-triazol-3-yl)phenyl]methyl}pyrrolidine-2-carboxamide;
1-acetyl-4-fluoro-N-{[6-fluoro-5-(propan-2-yl)pyridin-2-yl][3-(1H-pyrazol-5-yl)phenyl]methyl}pyrrolidine-2-carboxamide;
1-(2-acetamidoacetyl)-4-fluoro-N-{[6-fluoro-5-(propan-2-yl)pyridin-2-yl][3-(1H-pyrazol-5-yl)phenyl]methyl}pyrrolidine-2-carboxamide;
4-fluoro-N-{[6-fluoro-5-(propan-2-yl)pyridin-2-yl][3-(1,3-oxazol-5-yl)phenyl]methyl}-1-[2-(1H-1,2,3-triazol-5-yl)acetyl]pyrrolidine-2-carboxamide;
4-fluoro-N-{[6-fluoro-5-(propan-2-yl)pyridin-2-yl][3-(1,2-oxazol-5-yl)phenyl]methyl}-1-[2-(1H-1,2,3-triazol-5-yl)acetyl]pyrrolidine-2-carboxamide;
1-acetyl-4-fluoro-N-{[6-fluoro-5-(propan-2-yl)pyridin-2-yl][3-(1-methyl-1H-pyrazol-5-yl)phenyl]methyl}pyrrolidine-2-carboxamide;
1-acetyl-4-fluoro-N-{[6-fluoro-5-(propan-2-yl)pyridin-2-yl][3-(3-methyl-1H-pyrazol-5-yl)phenyl]methyl}pyrrolidine-2-carboxamide;
1-acetyl-N-{[3-(1,3-dimethyl-1H-pyrazol-5-yl)phenyl][6-fluoro-5-(propan-2-yl)pyridin-2-yl]methyl}-4-fluoropyrrolidine-2-carboxamide;
1-acetyl-4-fluoro-N-{[6-fluoro-5-(propan-2-yl)pyridin-2-yl][3-(1,2-oxazol-5-yl)phenyl]methyl}pyrrolidine-2-carboxamide;
1-acetyl-4-fluoro-N-{[6-fluoro-5-(propan-2-yl)pyridin-2-yl][3-(1H-pyrazol-1-yl)phenyl]methyl}pyrrolidine-2-carboxamide;
1-acetyl-4-fluoro-N-{[6-fluoro-5-(propan-2-yl)pyridin-2-yl][3-(1,3,4-oxadiazol-2-yl)phenyl]methyl}pyrrolidine-2-carboxamide;
1-(oct-7-ynoyl)-N-{phenyl[4-(propan-2-yl)phenyl]methyl}pyrrolidine-2-carboxamide;
4-fluoro-1-(1-methyl-1H-indazole-5-carbonyl)-N-{phenyl[4-(propan-2-yl)phenyl]methyl}pyrrolidine-2-carboxamide;
4-fluoro-1-[2-(4-methoxyphenyl)cyclopropanecarbonyl]-N-{phenyl[4-(propan-2-yl)phenyl]methyl}pyrrolidine-2-carboxamide;
4-fluoro-1-(7-oxo-5,6,7,8-tetrahydro-1,8-naphthyridine-2-carbonyl)-N-{phenyl[4-(propan-2-yl)phenyl]methyl}pyrrolidine-2-carboxamide;
4-fluoro-1-[2-(2-methylpropoxy)pyridine-4-carbonyl]-N-{phenyl[4-(propan-2-yl)phenyl]methyl}pyrrolidine-2-carboxamide;
4-fluoro-1-[3-(1H-imidazol-4-yl)propanoyl]-N-{phenyl[4-(propan-2-yl)phenyl]methyl}pyrrolidine-2-carboxamide;
4-fluoro-1-[4-(1H-imidazol-1-yl)pyridine-2-carbonyl]-N-{phenyl[4-(propan-2-yl)phenyl]methyl}pyrrolidine-2-carboxamide;
4-fluoro-N-{phenyl[4-(propan-2-yl)phenyl]methyl}-1-{[(pyridin-3-yl)carbamoyl]carbonyl}pyrrolidine-2-carboxamide;
4-fluoro-1-(5-methoxy-1-methyl-1H-pyrazole-3-carbonyl)-N-{phenyl[4-(propan-2-yl)phenyl]methyl}pyrrolidine-2-carboxamide;
1-[2-(1,4-dimethyl-5-oxo-4,5-dihydro-1H-1,2,4-triazol-3-yl)acetyl]-4-fluoro-N-{phenyl[5-(propan-2-yl)pyridin-2-yl]methyl}pyrrolidine-2-carboxamide;
1-{2-[4-(azetidin-1-yl)-1H-1,2,3-triazol-1-yl]acetyl}-4-fluoro-N-{phenyl[5-(propan-2-yl)pyridin-2-yl]methyl}pyrrolidine-2-carboxamide;
4-fluoro-1-[2-(5-methoxy-1-methyl-1H-1,2,4-triazol-3-yl)acetyl]-N-{phenyl[5-(propan-2-yl)pyridin-2-yl]methyl}pyrrolidine-2-carboxamide;
1-{2-[5-(diethylamino)-1H-1,2,3-triazol-1-yl]acetyl}-4-fluoro-N-{phenyl[5-(propan-2-yl)pyridin-2-yl]methyl}pyrrolidine-2-carboxamide;
1-{2-[5-(3,3-difluoroazetidin-1-yl)-1H-1,2,3-triazol-1-yl]acetyl}-4-fluoro-N-{phenyl[5-(propan-2-yl)pyridin-2-yl]methyl}pyrrolidine-2-carboxamide;
1-{2-[5-(azetidin-1-yl)-1H-1,2,3-triazol-1-yl]acetyl}-4-fluoro-N-{phenyl[5-(propan-2-yl)pyridin-2-yl]methyl}pyrrolidine-2-carboxamide;
N-[(5-cyclopropyl-6-fluoropyridin-2-yl)(phenyl)methyl]-4-fluoro-1-(2-{3-oxo-2H,3H-[1,2,4]triazolo[4,3-a]pyridin-8-yl}acetyl)pyrrolidine-2-carboxamide;
N-[(5-cyclopropyl-6-fluoropyridin-2-yl)(phenyl)methyl]-4-fluoro-1-[2-(4-methyl-5-oxo-4,5-dihydro-1,2,4-oxadiazol-3-yl)acetyl]pyrrolidine-2-carboxamide;
N-[(5-cyclopropyl-6-fluoropyridin-2-yl)(phenyl)methyl]-4-fluoro-1-[2-(1H-1,2,3-triazol-5-yl)acetyl]pyrrolidine-2-carboxamide;
N-[(5-cyclopropyl-6-fluoropyridin-2-yl)(phenyl)methyl]-1-[2-(1,4-dimethyl-5-oxo-4,5-dihydro-1H-1,2,4-triazol-3-yl)acetyl]-4-fluoropyrrolidine-2-carboxamide;
N-[(5-cyclopropyl-6-fluoropyridin-2-yl)(phenyl)methyl]-4-fluoro-1-[2-(1H-pyrazol-1-yl)acetyl]pyrrolidine-2-carboxamide;
N-[(5-cyclopropyl-6-fluoropyridin-2-yl)(phenyl)methyl]-4-fluoro-1-[2-(5-methyl-1H-pyrazol-1-yl)acetyl]pyrrolidine-2-carboxamide;
N-[(5-cyclopropyl-6-fluoropyridin-2-yl)(phenyl)methyl]-1-{2-[5-(difluoromethyl)-1H-pyrazol-1-yl]acetyl}-4-fluoropyrrolidine-2-carboxamide;
1-{2-[4-(azetidin-1-yl)-1H-1,2,3-triazol-1-yl]acetyl}-N-[(5-cyclopropyl-6-fluoropyridin-2-yl)(phenyl)methyl]-4-fluoropyrrolidine-2-carboxamide;
N-[(5-cyclopropyl-6-fluoropyridin-2-yl)(phenyl)methyl]-1-[2-(2,4-dioxo-1,2,3,4-tetrahydropyrimidin-1-yl)acetyl]-4-fluoropyrrolidine-2-carboxamide;
N-[(5-cyclopropyl-6-fluoropyridin-2-yl)(phenyl)methyl]-4-fluoro-1-{2-[4-(trifluoromethyl)-1H-pyrazol-1-yl]acetyl}pyrrolidine-2-carboxamide;
N-[(5-cyclopropyl-6-fluoropyridin-2-yl)(phenyl)methyl]-1-{2-[3-(difluoromethyl)-1H-pyrazol-1-yl]acetyl}-4-fluoropyrrolidine-2-carboxamide;
N-[(5-cyclopropyl-6-fluoropyridin-2-yl)(phenyl)methyl]-4-fluoro-1-{2-[3-(trifluoromethyl)-1H-pyrazol-1-yl]acetyl}pyrrolidine-2-carboxamide;
N-[(5-cyclopropyl-6-fluoropyridin-2-yl)(phenyl)methyl]-4-fluoro-1-[2-(2-oxo-1,2-dihydropyridin-3-yl)acetyl]pyrrolidine-2-carboxamide;
N-[(5-cyclopropyl-6-fluoropyridin-2-yl)(phenyl)methyl]-4-fluoro-1-[2-(5-methyl-6-oxo-1,6-dihydropyridin-3-yl)acetyl]pyrrolidine-2-carboxamide;
N-[(5-cyclopropyl-6-fluoropyridin-2-yl)(phenyl)methyl]-1-[2-(3-ethyl-5-methyl-2,4-dioxo-1,2,3,4-tetrahydropyrimidin-1-yl)acetyl]-4-fluoropyrrolidine-2-carboxamide;
N-[(5-cyclopropyl-6-fluoropyridin-2-yl)(phenyl)methyl]-4-fluoro-1-[2-(6-oxo-1,6-dihydropyridin-3-yl)acetyl]pyrrolidine-2-carboxamide;
N-[(5-cyclopropyl-6-fluoropyridin-2-yl)(phenyl)methyl]-1-[2-(3-ethyl-2,4-dioxo-1,2,3,4-tetrahydropyrimidin-1-yl)acetyl]-4-fluoropyrrolidine-2-carboxamide;
N-[(5-cyclopropyl-6-fluoropyridin-2-yl)(phenyl)methyl]-1-[2-(1-ethyl-6-oxo-1,6-dihydropyridin-3-yl)acetyl]-4-fluoropyrrolidine-2-carboxamide;
N-[(5-cyclopropyl-6-fluoropyridin-2-yl)(phenyl)methyl]-1-[2-(6-ethoxy-5-methylpyridin-3-yl)acetyl]-4-fluoropyrrolidine-2-carboxamide;
N-[(5-cyclopropyl-6-fluoropyridin-2-yl)(phenyl)methyl]-4-fluoro-1-{2-[4-(trifluoromethyl)-1,3-oxazol-2-yl]acetyl}pyrrolidine-2-carboxamide;
N-[(5-cyclopropyl-6-fluoropyridin-2-yl)(phenyl)methyl]-1-[2-(1-ethyl-5-methyl-6-oxo-1,6-dihydropyridin-3-yl)acetyl]-4-fluoropyrrolidine-2-carboxamide;
N-[(5-cyclopropyl-6-fluoropyridin-2-yl)(phenyl)methyl]-1-[2-(1-ethyl-5-methyl-2-oxo-1,2-dihydropyridin-3-yl)acetyl]-4-fluoropyrrolidine-2-carboxamide;
N-[(5-cyclopropyl-6-fluoropyridin-2-yl)(phenyl)methyl]-1-[2-(1-ethyl-2-oxo-1,2-dihydropyridin-3-yl)acetyl]-4-fluoropyrrolidine-2-carboxamide;
N-[(5-cyclopropyl-6-fluoropyridin-2-yl)(phenyl)methyl]-1-[2-(4-ethyl-5-oxo-4,5-dihydropyrazin-2-yl)acetyl]-4-fluoropyrrolidine-2-carboxamide;
N-[(5-cyclopropyl-6-fluoropyridin-2-yl)(phenyl)methyl]-4-fluoro-1-[2-(5-methoxy-1-methyl-1H-1,2,4-triazol-3-yl)acetyl]pyrrolidine-2-carboxamide;
1-(2-{[benzyl(trifluoromethyl)carbamoyl]amino}acetyl)-N-[(5-cyclopropyl-6-fluoropyridin-2-yl)(phenyl)methyl]-4-fluoropyrrolidine-2-carboxamide;
N-[(5-cyclopropyl-6-fluoropyridin-2-yl)(phenyl)methyl]-4-fluoro-1-{2-[5-(trifluoromethyl)-1H-1,2,3-triazol-1-yl]acetyl}pyrrolidine-2-carboxamide;
N-[(5-cyclopropyl-6-fluoropyridin-2-yl)(phenyl)methyl]-4-fluoro-1-[2-(5-oxo-4,5-dihydropyrazin-2-yl)acetyl]pyrrolidine-2-carboxamide;
N-[(5-cyclopropyl-6-fluoropyridin-2-yl)(phenyl)methyl]-1-{2-[5-(difluoromethyl)-1H-1,2,3-triazol-1-yl]acetyl}-4-fluoropyrrolidine-2-carboxamide;
N-[(5-cyclopropyl-6-fluoropyridin-2-yl)(phenyl)methyl]-1-{2-[5-(diethylamino)-1H-1,2,3-triazol-1-yl]acetyl}-4-fluoropyrrolidine-2-carboxamide;
N-[(5-cyclopropyl-6-fluoropyridin-2-yl)(phenyl)methyl]-1-{2-[3-(difluoromethyl)-1-methyl-1H-pyrazol-4-yl]acetyl}-4-fluoropyrrolidine-2-carboxamide;
N-[(5-cyclopropyl-6-fluoropyridin-2-yl)(phenyl)methyl]-1-{2-[5-(difluoromethyl)-3-methyl-1H-pyrazol-1-yl]acetyl}-4-fluoropyrrolidine-2-carboxamide;
N-[(5-cyclopropyl-6-fluoropyridin-2-yl)(phenyl)methyl]-1-{2-[5-(difluoromethyl)-1-methyl-1H-pyrazol-4-yl]acetyl}-4-fluoropyrrolidine-2-carboxamide;
N-[(5-cyclopropyl-6-fluoropyridin-2-yl)(phenyl)methyl]-1-{2-[3-(difluoromethyl)-5-methyl-1H-pyrazol-1-yl]acetyl}-4-fluoropyrrolidine-2-carboxamide;
N-[(5-cyclopropyl-6-fluoropyridin-2-yl)(phenyl)methyl]-1-[2-(4-ethyl-3-oxo-3,4-dihydropyrazin-2-yl)acetyl]-4-fluoropyrrolidine-2-carboxamide;
N-[(5-cyclopropyl-6-fluoropyridin-2-yl)(phenyl)methyl]-1-{2-[4-(difluoromethyl)-1-methyl-1H-pyrazol-5-yl]acetyl}-4-fluoropyrrolidine-2-carboxamide;
N-[(5-cyclopropyl-6-fluoropyridin-2-yl)(phenyl)methyl]-4-fluoro-1-{2-[5-(trifluoromethyl)-1H-1,2,3,4-tetrazol-1-yl]acetyl}pyrrolidine-2-carboxamide;
N-[(5-cyclopropyl-6-fluoropyridin-2-yl)(phenyl)methyl]-1-{2-[4-(difluoromethyl)-1H-1,2,3-triazol-5-yl]acetyl}-4-fluoropyrrolidine-2-carboxamide;
N-[(5-cyclopropyl-6-fluoropyridin-2-yl)(phenyl)methyl]-1-{2-[4-(difluoromethyl)-1-methyl-1H-pyrazol-3-yl]acetyl}-4-fluoropyrrolidine-2-carboxamide;
N-[(5-cyclopropyl-6-fluoropyridin-2-yl)(phenyl)methyl]-1-{2-[5-(3,3-difluoroazetidin-1-yl)-1H-1,2,3-triazol-1-yl]acetyl}-4-fluoropyrrolidine-2-carboxamide;
N-[(5-cyclopropyl-6-fluoropyridin-2-yl)(phenyl)methyl]-1-{2-[3-(difluoromethyl)-4H-1,2,4-triazol-4-yl]acetyl}-4-fluoropyrrolidine-2-carboxamide;
N-[(5-cyclopropyl-6-fluoropyridin-2-yl)(phenyl)methyl]-4-fluoro-1-{2-[5-(trifluoromethyl)-1H-pyrazol-1-yl]acetyl}pyrrolidine-2-carboxamide;
N-[(5-cyclopropyl-6-fluoropyridin-2-yl)(phenyl)methyl]-1-[2-(4,5-dimethyl-1,3-oxazol-2-yl)acetyl]-4-fluoropyrrolidine-2-carboxamide;
1-{2-[5-(azetidin-1-yl)-1H-1,2,3-triazol-1-yl]acetyl}-N-[(5-cyclopropyl-6-fluoropyridin-2-yl)(phenyl)methyl]-4-fluoropyrrolidine-2-carboxamide;
N-[(5-cyclopropyl-6-fluoropyridin-2-yl)(phenyl)methyl]-1-{2-[5-(difluoromethyl)-1H-1,2,3,4-tetrazol-1-yl]acetyl}-4-fluoro-3-hydroxypyrrolidine-2-carboxamide;
1-(3-carbamoyl-2-acetamidopropanoyl)-4-fluoro-N-{[3-fluoro-4-(propan-2-yl)phenyl](phenyl)methyl}pyrrolidine-2-carboxamide;
4-fluoro-N-{[3-fluoro-4-(propan-2-yl)phenyl](phenyl)methyl}-1-[2-(1H-1,2,3,4-tetrazol-1-yl)propanoyl]pyrrolidine-2-carboxamide;
4-fluoro-N-{[3-fluoro-4-(propan-2-yl)phenyl](phenyl)methyl}-1-[2-(1H-imidazol-1-yl)propanoyl]pyrrolidine-2-carboxamide;
4-fluoro-N-{[3-fluoro-4-(propan-2-yl)phenyl](phenyl)methyl}-1-[2-(1H-1,2,3-triazol-5-yl)propanoyl]pyrrolidine-2-carboxamide;
4-fluoro-N-{[3-fluoro-4-(propan-2-yl)phenyl](phenyl)methyl}-1-[(1H-1,2,3-triazol-5-yl)methanesulfonyl]pyrrolidine-2-carboxamide;
4-fluoro-N-{[3-fluoro-4-(propan-2-yl)phenyl](phenyl)methyl}-1-[2-hydroxy-2-(1H-1,2,3-triazol-5-yl)acetyl]pyrrolidine-2-carboxamide;
1-{2-[4-(dimethylamino)-1H-1,2,3-triazol-5-yl]acetyl}-4-fluoro-N-{[3-fluoro-4-(propan-2-yl)phenyl](phenyl)methyl}pyrrolidine-2-carboxamide;
4-fluoro-N-{[6-fluoro-5-(propan-2-yl)pyridin-2-yl](phenyl)methyl}-1-[2-(1H-1,2,3-triazol-5-yl)acetyl]pyrrolidine-2-carboxamide;
1-[2-(3,5-dimethyl-2,4-dioxo-1,2,3,4-tetrahydropyrimidin-1-yl)acetyl]-4-fluoro-N-{[6-fluoro-5-(propan-2-yl)pyridin-2-yl](phenyl)methyl}pyrrolidine-2-carboxamide;
4-fluoro-N-{[6-fluoro-5-(propan-2-yl)pyridin-2-yl](phenyl)methyl}-1-[2-(5-methyl-2,4-dioxo-1,2,3,4-tetrahydropyrimidin-1-yl)acetyl]pyrrolidine-2-carboxamide;
1-[2-(1,4-dimethyl-5-oxo-4,5-dihydro-1H-1,2,4-triazol-3-yl)acetyl]-4-fluoro-N-{[6-fluoro-5-(propan-2-yl)pyridin-2-yl](phenyl)methyl}pyrrolidine-2-carboxamide;
4-fluoro-N-{[6-fluoro-5-(propan-2-yl)pyridin-2-yl](phenyl)methyl}-1-(3-{3-oxo-2H,3H-[1,2,4]triazolo[4,3-a]pyridin-2-yl}propanoyl)pyrrolidine-2-carboxamide;
4-fluoro-N-{[6-fluoro-5-(propan-2-yl)pyridin-2-yl](phenyl)methyl}-1-[2-(1H-pyrazol-1-yl)acetyl]pyrrolidine-2-carboxamide;
4-fluoro-N-{[6-fluoro-5-(propan-2-yl)pyridin-2-yl](phenyl)methyl}-1-[2-(5-methyl-1H-pyrazol-1-yl)acetyl]pyrrolidine-2-carboxamide;
1-{2-[5-(difluoromethyl)-1H-pyrazol-1-yl]acetyl}-4-fluoro-N-{[6-fluoro-5-(propan-2-yl)pyridin-2-yl](phenyl)methyl}pyrrolidine-2-carboxamide;
4-fluoro-N-{[6-fluoro-5-(propan-2-yl)pyridin-2-yl](phenyl)methyl}-1-[2-(5-methyl-6-oxo-1,6-dihydropyridin-3-yl)acetyl]pyrrolidine-2-carboxamide;
1-[2-(2,4-dioxo-1,2,3,4-tetrahydropyrimidin-1-yl)acetyl]-4-fluoro-N-{[6-fluoro-5-(propan-2-yl)pyridin-2-yl](phenyl)methyl}pyrrolidine-2-carboxamide;
1-[2-(3-ethyl-2,4-dioxo-1,2,3,4-tetrahydropyrimidin-1-yl)acetyl]-4-fluoro-N-{[6-fluoro-5-(propan-2-yl)pyridin-2-yl](phenyl)methyl}pyrrolidine-2-carboxamide;
1-{2-[4-(azetidin-1-yl)-1H-1,2,3-triazol-1-yl]acetyl}-4-fluoro-N-{[6-fluoro-5-(propan-2-yl)pyridin-2-yl](phenyl)methyl}pyrrolidine-2-carboxamide;
4-fluoro-N-{[6-fluoro-5-(propan-2-yl)pyridin-2-yl](phenyl)methyl}-1-{2-[4-(trifluoromethyl)-1H-pyrazol-1-yl]acetyl}pyrrolidine-2-carboxamide;
1-{2-[3-(difluoromethyl)-1H-pyrazol-1-yl]acetyl}-4-fluoro-N-{[6-fluoro-5-(propan-2-yl)pyridin-2-yl](phenyl)methyl}pyrrolidine-2-carboxamide;
4-fluoro-N-{[6-fluoro-5-(propan-2-yl)pyridin-2-yl](phenyl)methyl}-1-{2-[3-(trifluoromethyl)-1H-pyrazol-1-yl]acetyl}pyrrolidine-2-carboxamide;
4-fluoro-N-{[6-fluoro-5-(propan-2-yl)pyridin-2-yl](phenyl)methyl}-1-[2-(6-oxo-1,6-dihydropyridin-3-yl)acetyl]pyrrolidine-2-carboxamide;
1-[2-(1-ethyl-6-oxo-1,6-dihydropyridin-3-yl)acetyl]-4-fluoro-N-{[6-fluoro-5-(propan-2-yl)pyridin-2-yl](phenyl)methyl}pyrrolidine-2-carboxamide;
4-fluoro-N-{[6-fluoro-5-(propan-2-yl)pyridin-2-yl](phenyl)methyl}-1-[2-(2-oxo-1,2-dihydropyridin-3-yl)acetyl]pyrrolidine-2-carboxamide;
1-[2-(3-ethyl-5-methyl-2,4-dioxo-1,2,3,4-tetrahydropyrimidin-1-yl)acetyl]-4-fluoro-N-{[6-fluoro-5-(propan-2-yl)pyridin-2-yl](phenyl)methyl}pyrrolidine-2-carboxamide;
1-[2-(1-ethyl-2-oxo-1,2-dihydropyridin-3-yl)acetyl]-4-fluoro-N-{[6-fluoro-5-(propan-2-yl)pyridin-2-yl](phenyl)methyl}pyrrolidine-2-carboxamide;
1-[2-(1-ethyl-5-methyl-6-oxo-1,6-dihydropyridin-3-yl)acetyl]-4-fluoro-N-{[6-fluoro-5-(propan-2-yl)pyridin-2-yl](phenyl)methyl}pyrrolidine-2-carboxamide;
1-[2-(4-ethyl-5-oxo-4,5-dihydropyrazin-2-yl)acetyl]-4-fluoro-N-{[6-fluoro-5-(propan-2-yl)pyridin-2-yl](phenyl)methyl}pyrrolidine-2-carboxamide;
1-[2-(6-ethoxy-5-methylpyridin-3-yl)acetyl]-4-fluoro-N-{[6-fluoro-5-(propan-2-yl)pyridin-2-yl](phenyl)methyl}pyrrolidine-2-carboxamide;
1-[2-(1-ethyl-5-methyl-2-oxo-1,2-dihydropyridin-3-yl)acetyl]-4-fluoro-N-{[6-fluoro-5-(propan-2-yl)pyridin-2-yl](phenyl)methyl}pyrrolidine-2-carboxamide;
4-fluoro-N-{[6-fluoro-5-(propan-2-yl)pyridin-2-yl](phenyl)methyl}-1-[2-(5-methoxy-1-methyl-1H-1,2,4-triazol-3-yl)acetyl]pyrrolidine-2-carboxamide;
4-fluoro-N-{[6-fluoro-5-(propan-2-yl)pyridin-2-yl](phenyl)methyl}-1-{2-[4-(trifluoromethyl)-1,3-oxazol-2-yl]acetyl}pyrrolidine-2-carboxamide;
4-fluoro-N-{[6-fluoro-5-(propan-2-yl)pyridin-2-yl](phenyl)methyl}-1-[2-(5-oxo-4,5-dihydropyrazin-2-yl)acetyl]pyrrolidine-2-carboxamide;
1-{2-[5-(difluoromethyl)-3-methyl-TH-pyrazol-1-yl]acetyl}-4-fluoro-N-{[6-fluoro-5-(propan-2-yl)pyridin-2-yl](phenyl)methyl}pyrrolidine-2-carboxamide;
1-{2-[3-(difluoromethyl)-5-methyl-TH-pyrazol-1-yl]acetyl}-4-fluoro-N-{[6-fluoro-5-(propan-2-yl)pyridin-2-yl](phenyl)methyl}pyrrolidine-2-carboxamide;
1-{2-[5-(diethylamino)-1H-1,2,3-triazol-1-yl]acetyl}-4-fluoro-N-{[6-fluoro-5-(propan-2-yl)pyridin-2-yl](phenyl)methyl}pyrrolidine-2-carboxamide;
1-[2-(4-ethyl-3-oxo-3,4-dihydropyrazin-2-yl)acetyl]-4-fluoro-N-{[6-fluoro-5-(propan-2-yl)pyridin-2-yl](phenyl)methyl}pyrrolidine-2-carboxamide;
1-{2-[5-(3,3-difluoroazetidin-1-yl)-1H-1,2,3-triazol-1-yl]acetyl}-4-fluoro-N-{[6-fluoro-5-(propan-2-yl)pyridin-2-yl](phenyl)methyl}pyrrolidine-2-carboxamide;
1-{2-[5-(azetidin-1-yl)-1H-1,2,3-triazol-1-yl]acetyl}-4-fluoro-N-{[6-fluoro-5-(propan-2-yl)pyridin-2-yl](phenyl)methyl}pyrrolidine-2-carboxamide;
1-[2-(1H-1,3-benzodiazol-1-yl)propanoyl]-N-[(4-cyclopropyl-3-fluorophenyl)(phenyl)methyl]-4-fluoropyrrolidine-2-carboxamide;
   wherein, the (G1-Z1) and (G2-Z2) moieties are each independently optionally substituted with a group bound to L;
   or a stereoisomer or tautomer thereof, or a pharmaceutically acceptable salt of any of the foregoing.

In some embodiments of a compound of formula (I), or any variation or embodiment thereof, or a stereoisomer or tautomer thereof, or a pharmaceutically acceptable salt of any of the foregoing, G1 and G2 are each independently selected from the moiety of the compounds of Table 1A represented by

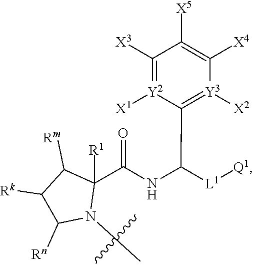

or the moiety of the compounds of Table 1A represented by

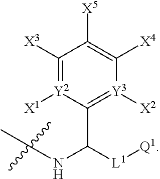

For example, in some embodiments G1 is the moiety of

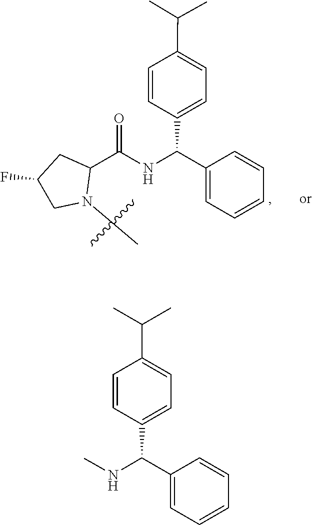 [SMILES: *N1C[C@H](F)CC1C(=O)N[C@@H](c1ccccc1)c1ccc(C(C)C)cc1 ; CN[C@@H](c1ccccc1)c1ccc(C(C)C)cc1]

of compound GZ-1

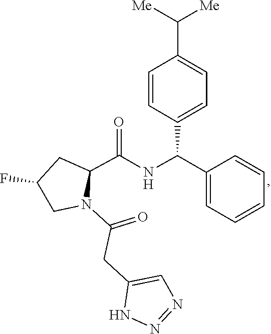 [SMILES: CC(C)c1ccc([C@@H](NC(=O)[C@@H]2C[C@@H](F)CN2C(=O)Cc2cnn[nH]2)c2ccccc2)cc1]

### in Table 1A.

In some embodiments of a compound of formula (I), or a stereoisomer or tautomer thereof, or a pharmaceutically acceptable salt of any of the foregoing, (Z1) and (Z2) are each, independently, -L²-R², wherein L² and R² are as defined for a compound of formula (I-1), or (I-2). In some embodiments, L² and R² are as defined for a compound of formula (I), (I-A), (I-A1), (I-A2), (I-A3), (I-A4), (I-B), (I-B1), (I-B32), (I-C), (I-D), (I-D1), (I-D32), (I-E), (I-F), (I-G), or (I-H).

In one aspect, provided herein is a compound of formula (I), wherein the compound is a compound of formula (II):

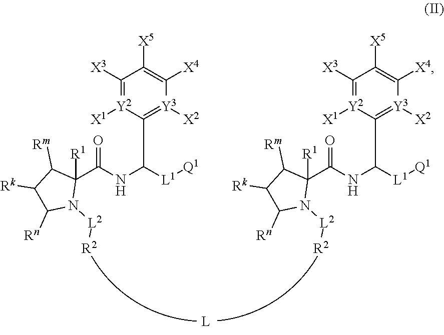

or a stereoisomer or tautomer thereof, or a pharmaceutically acceptable salt of any of the foregoing, wherein L, is as defined for a compound of formula (I) and wherein L¹, L², Y², Y³, R¹, R², R^{k}, R^{m}, Rⁿ, X¹, X², X³, X⁴, X⁵, and Q¹ are as defined for a compound of formula (I-1).

In one aspect, provided herein is a compound of formula (I), or (II), wherein the compound is a compound of formula (II-A):

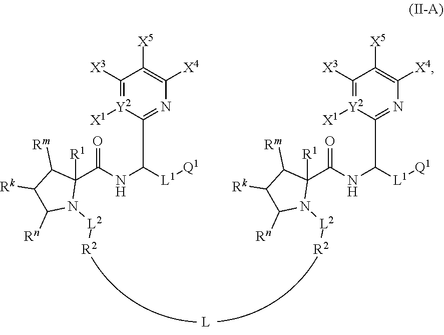

or a stereoisomer or tautomer thereof, or a pharmaceutically acceptable salt of any of the foregoing, wherein L, is as defined for a compound of formula (I) and wherein L¹, L², Y², R¹, R², R^{k}, R^{m}, Rⁿ, X¹, X³, X⁴, X⁵, and Q¹ are as defined for a compound of formula (I-1).

In one aspect, provided herein is a compound of formula (I), wherein the compound is a compound of formula (III):

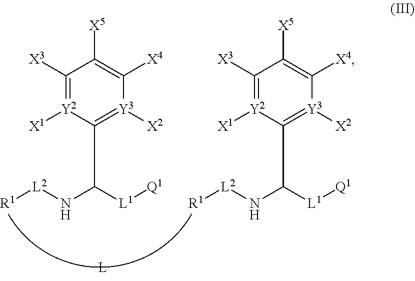

or a stereoisomer or tautomer thereof, or a pharmaceutically acceptable salt of any of the foregoing, wherein L, is as defined for a compound of formula (I) and wherein L¹, L², Y², Y³, R¹, R², R^{k}, R^{m}, Rⁿ, X¹, X², X³, X⁴, X⁵, and Q¹ are as defined for a compound of formula (I-2).

In one aspect, provided herein is a compound of formula (I), or (III) wherein the compound is a compound of formula (III-A):

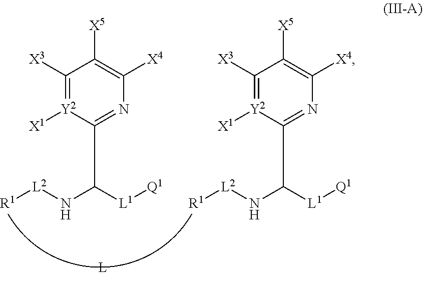

or a stereoisomer or tautomer thereof, or a pharmaceutically acceptable salt of any of the foregoing, wherein L, is as defined for a compound of formula (I) and wherein L¹, L², Y², R¹, R², R^{k}, R^{m}, Rⁿ, X¹, X³, X⁴, X⁵, and Q¹ are as defined for a compound of formula (I-2).

In one aspect, provided herein is a compound of formula (I), wherein the compound is a compound of formula (IV):

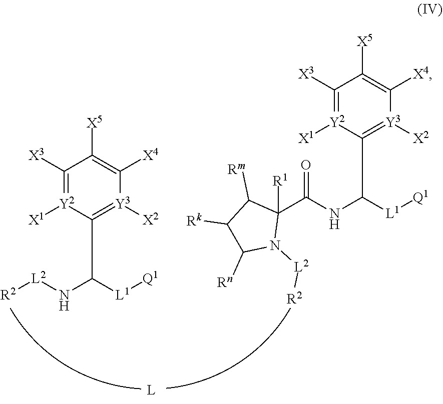

or a stereoisomer or tautomer thereof, or a pharmaceutically acceptable salt of any of the foregoing, wherein L, is as defined for a compound of formula (I) and wherein L¹, L², Y², Y³, R¹, R², R^{k}, R^{m}, Rⁿ, X¹, X², X³, X⁴, X⁵, and Q¹ are as defined for a compound of formula (I-1), or (I-2).

In one aspect, provided herein is a compound of formula (I), or (IV), wherein the compound is a compound of formula (IV-A):

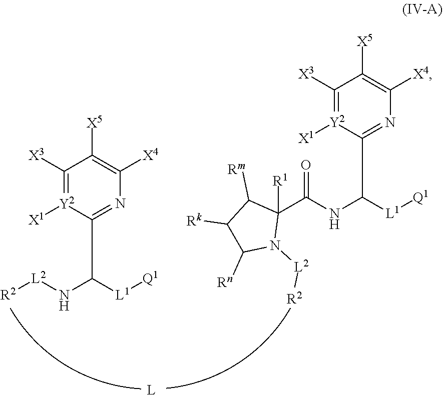

or a stereoisomer or tautomer thereof, or a pharmaceutically acceptable salt of any of the foregoing, wherein L, is as defined for a compound of formula (I) and wherein L¹, L², Y², Y³, R¹, R², R^{k}, R^{m}, Rⁿ, X¹, X², X³, X⁴, X⁵, and Q¹ are as defined for a compound of formula (I-1), or (I-2).

In one aspect, provided herein is a compound of formula (I), wherein the compound is a compound of formula (V):

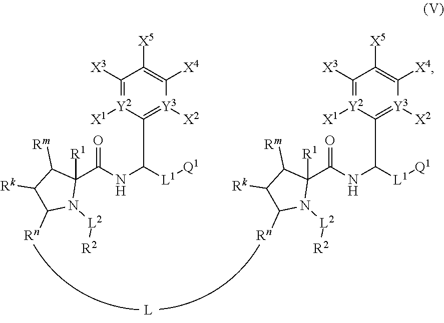

or a stereoisomer or tautomer thereof, or a pharmaceutically acceptable salt of any of the foregoing, wherein L, is as defined for a compound of formula (I) and wherein L¹, L², Y², Y³, R¹, R², R^{k}, R^{m}, Rⁿ, X¹, X², X³, X⁴, X⁵, and Q¹ are as defined for a compound of formula (I-1)

In one aspect, provided herein is a compound of formula (I), or (V), wherein the compound is a compound of formula (V-A):

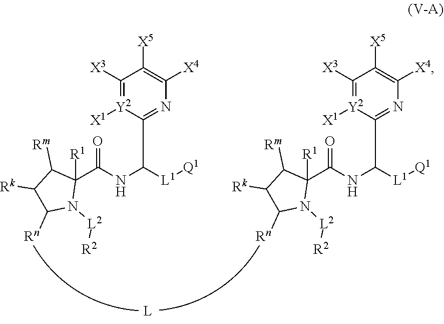

or a stereoisomer or tautomer thereof, or a pharmaceutically acceptable salt of any of the foregoing, wherein L, is as defined for a compound of formula (I) and wherein L¹, L², Y², Y³, R¹, R², R^{k}, R^{m}, Rⁿ, X¹, X², X³, X⁴, X⁵, and Q¹ are as defined for a compound of formula (I-1)

In some embodiments of a compound of formula (I), such as a compound of formula (II), (IV), or (V), or any variation or embodiment thereof, or a stereoisomer or tautomer thereof, or a pharmaceutically acceptable salt of any of the foregoing, wherein independently for each L, L¹, L², Y², Y³, R¹, R², R^{k}, R^{m}, Rⁿ, X¹, X², X³, X⁴, X⁵, and Q¹:
Y² and Y³ are each C, or
one of Y² and Y³ is N and the other of Y² and Y³ is C;
X¹ and X² are each independently H, C₁₋₆alkyl, or C₁₋₆alkoxy;
X³ and X⁴ are each independently H, halo, C₁₋₆alkyl, C₁₋₆alkoxy, or 5-20 membered heteroaryl, wherein the C₁₋₆alkyl of X³ and X⁴ is optionally substituted with one of more halo;
X⁵ is H, C₁₋₆alkyl, C₁₋₆alkoxy, or C₃₋₁₀cycloalkyl;
either
(1) L¹ is absent; and
Q¹ is selected from (i) to (iv):
(i) phenyl, wherein the phenyl of Q¹ is substituted with one or more halo, C₁₋₆alkyl, C₂₋₆alkenyl, —NH₂, —NH—C(O)—(C₁₋₆alkyl), —NH—C(O)-(3-15 membered heterocyclyl), C₃₋₁₀cycloalkyl, or 5-20 membered heteroaryl, wherein
   the C₁₋₆alkyl is optionally substituted with one or more halo, —NH—C(O)—NH(C₁₋₆alkyl), —NH—C(O)—C₁₋₆alkyl, or —NH—C(O)—C₁₋₆alkoxy,
   the C₃₋₁₀cycloalkyl is optionally substituted with one or more halo or C₁₋₆alkyl, and
   the 5-20 membered heteroaryl is optionally substituted with one or more C₁₋₆alkyl,
(ii) 3-15 membered heterocyclyl, wherein the 3-15 membered heterocyclyl of Q¹ is optionally substituted with one or more oxo, or C₁₋₆alkyl,
(iii) 5-20 membered heteroaryl, wherein the 5-20 membered heteroaryl of Q¹ is optionally substituted with one or more halo, C₁₋₆alkyl, C₂₋₆alkenyl, C₁₋₆alkoxy, —NH₂, or C₃₋₁₀cycloalkyl, wherein,
   the C₁₋₆alkyl is optionally substituted with one or more halo, and
   the C₃₋₁₀cycloalkyl is optionally substituted with one or more halo or C₁₋₆alkyl, and
(iv) C₃₋₁₀cycloalkyl;
or
(2) L¹ is —CH₂—; and
Q¹ is C₃₋₁₀cycloalkyl;
L² is —C(O)— or —S(O)₂—
R¹ is H or C₁₋₆alkyl;
R^{k} is H, halo, —OH, —NH₂, or —NH—C(O)C₁₋₆alkyl;
R^{m} is H, —OH, or C₁₋₆alkyl;
Rⁿ is H, C₁₋₆alkyl, or C₃₋₁₀cycloalkyl or Rⁿ taken together with the carbon atom to which it is attached forms C₃₋₅ cycloalkyl;
or R^{k} is taken together with either R^{m} or Rⁿ, and the atoms to which they are attached, to form cyclopropyl; and
R² is selected from (i) to (vii):
(i) C₁₋₆alkyl, wherein the C₁₋₆alkyl of R² is optionally substituted with one or more R^{a}, wherein R^{a} is:
   (a) —OH,
   (b) cyano,
   (c) C₂₋₆alkynyl,
   (d) C₆₋₂₀aryl, wherein the C₆₋₂₀aryl of R^{a} is optionally substituted with one or more halo, cyano, C₁₋₆alkoxy, or —NH—C(O)—C₁₋₆alkyl,
   (e) 3-15 membered heterocyclyl, wherein the 3-15 membered heterocyclyl of R^{a} is optionally substituted with one or more R^{c}, wherein
      R^{co} is halo, oxo, C₁₋₆alkyl, C₁₋₆alkoxy, —C(O)—C₁₋₆alkyl, or —C(O)—C₁₋₆alkoxy, wherein
         the C₁₋₆alkyl of R^{c} is optionally substituted with one or more halo or C₂₋₆alkynyl, and
         the —C(O)—C₁₋₆alkoxy of R^{c} is optionally substituted with one or more halo,
   (f) —N(R^{c})(R^{d}), wherein R^{c} and R^{d} of —N(R^{c})(R^{d}) are, independently of each other, H, C₁₋₆alkyl, —C(O)—C₁₋₆alkyl, —C(O)—C₁₋₆alkoxy, —C(O)—NH₂, —C(O)—NH(C₁₋₆alkyl), —C(O)—N(C₁₋₆alkyl)₂, —C(O)-(3-15 membered heterocyclyl), —CH₂—C(O)—NH₂, 3-15 membered heterocyclyl, or 5-20 membered heteroaryl, wherein
      the C₁₋₆alkyl of R^{c} or R^{d} is optionally substituted with one or more —C(O)—NH₂,
      the —C(O)—C₁₋₆alkyl of R^{c} or R^{d} is optionally substituted with one or more halo,
      the 3-15 membered heterocyclyl and the 5-20 membered heteroaryl of R^{c} or R^{d} are independently optionally substituted with one or more C₁₋₆alkyl,
      the —C(O)-(3-15 membered heterocyclyl) of R^{c} or R^{d} is optionally substituted with one or more halo, —C(O)—C₁₋₆alkoxy, or C₁₋₆alkyl, wherein the C₁₋₆alkyl is optionally substituted with one or more halo, C₁₋₆alkoxy, or C₃₋₁₀cycloalkyl, and
      the C₁₋₆alkyl of the —C(O)—N(C₁₋₆alkyl)₂ of R^{c} or R^{d} are, independently of each other, optionally substituted with one or more halo or C₆₋₂₀aryl,
   (g) —O—R^{e}, wherein R^{e} is C₁₋₆alkyl, C₆₋₂₀aryl, —C(O)-(3-15 membered heterocyclyl), —C(O)—N—(C₁₋₆alkyl)₂, or 5-20 membered heteroaryl, wherein
      the C₁₋₆alkyl of R^{e} is optionally substituted with one or more C₁₋₆alkoxy, wherein the C₁₋₆alkoxy is optionally substituted with one or more C₂₋₆alkynyl,
      the C₆₋₂₀aryl of R^{e} is optionally substituted with one or more C₁₋₆alkyl, and
      the —C(O)-(3-15 membered heterocyclyl) of R^{e} is optionally substituted with one or more C₁₋₆alkyl, C₁₋₆alkoxy, or —C(O)—C₁₋₆alkoxy, wherein the C₁₋₆alkyl is optionally substituted with one or more halo, C₁₋₆alkoxy, or C₃₋₁₀cycloalkyl,
   (h) —C(O)—R^{e}, wherein R^{e} is —NH₂, —OH, or 3-15 membered heterocyclyl, or
   (i) —S(O)₂—R^{f}, wherein R^{f} is C₁₋₆alkyl or 3-15 membered heterocyclyl,
(ii) C₃₋₁₀cycloalkyl, wherein the C₃₋₁₀cycloalkyl of R² is optionally substituted with one or more R^{q}, wherein R^{q} is 5-20 membered heteroaryl or C₆₋₂₀aryl, wherein the C₆₋₂₀aryl of R^{q} is optionally substituted with one or more C₁₋₆alkoxy,
(iii) 3-15 membered heterocyclyl, wherein the 3-15 membered heterocyclyl of R² is optionally substituted with one or more halo, oxo, C₁₋₆alkyl, —C(O)—C₁₋₆alkyl, or 5-20 membered heteroaryl,
(iv) 5-20 membered heteroaryl or —(C₁₋₄alkyl)(5-20 membered heteroaryl), wherein the C₁₋₄ alkyl is optionally substituted with one or more or more —OH, halo, —NH₂, —NH(C₁₋₆alkyl), —N(C₁₋₆alkyl)₂, and wherein the 5-20 membered heteroaryl of the 5-20 membered heteroaryl or —(C₁₋₄alkyl)(5-20 membered heteroaryl), is optionally substituted with one or more R^{s}, wherein
   R^{s} is halo, C₁₋₆alkyl, C₁₋₆alkoxy, —NH₂, —NH(C₁₋₆alkyl), —N(C₁₋₆alkyl)₂, —NH—C(O)—C₁₋₆alkyl, C₆₋₂₀aryl, C₃₋₁₀cycloalkyl, 3-15 membered heterocyclyl, 5-20 membered heteroaryl, or —C(O)—C₁₋₆alkoxy, wherein
      the C₁₋₆alkyl of R^{s} is optionally substituted with one or more halo, C₁₋₆alkoxy, —NH₂, —NH(C₁₋₆alkyl), —N(C₁₋₆alkyl)₂, —NH—C(O)C₁₋₆alkyl, or —NH—C(O)—C₁₋₆alkoxy, and
      the 3-15-membered heterocyclyl of R^{s} is optionally substituted with one or more halo or —C(O)—C₁₋₆alkoxy,
(v) —N(R^{g})(R^{h}), wherein R⁹ and R^{h} are independently H or C₁₋₆alkyl,
(vi) —C(O)—R^{j}, wherein R^{j} is C₃₋₁₀cycloalkyl, —NH(C₁₋₆alkyl), —N(C₁₋₆alkyl)₂, or —NH(5-20 membered heteroaryl), and
(vii) C₆₋₂₀aryl, wherein the C₆₋₂₀aryl of R² is optionally substituted with one or more 5-20 membered heteroaryl or —O—R^{p}, wherein R^{p} is 3-15 membered heterocyclyl, wherein the 3-15 membered heterocyclyl of R^{p} is optionally substituted with one or more —C(O)—C₁₋₆alkyl;
L is selected from the group consisting of a bond or (C₁-C₅₀)alkyl, wherein
   the (C₁-C₅₀)alkyl is optionally substituted with one or more deutero, halo, (C₁-C₆)alkyl, (C₁-C₆)haloalkyl, oxo (C═O), —O(C₁-C₆)alkyl, —O(C₁-C₆)haloalkyl, —O(C₁-C₆)cycloalkyl, —N(R^{x})(R^{y})—, C₃-C₁₅ carbocyclcyl, 3- to 15-membered heterocyclcyl, C₆-C₁₄ aryl, or 5- to 20-membered heteroaryl; and
   one or more of the C atoms in the (C₁-C₅₀)alkyl is optionally replaced with one or more —C(R^{x})═C(R^{x})—, —C≡C—, —O—, —C(O)—, —C(O)O—, —OC(O)—, —OC(O)O—, —OC(O)N(R^{x})—, —N(R^{x})C(O)O—, —N(R^{x})C(O)N(R^{y})—, —C(O)N(R^{x})—, —N(R^{x})C(O)—, —N(R^{x})—, —S(O)ₙ—, —S(O)ₙO—, —OS(O)ₙ—, —OS(O)ₙO—, —OS(O)ₙN(R^{x})—, —N(R^{x})S(O)ₙO—, —N(R^{x})S(O)ₙN(R^{y})—, —S(O)ₙN(R^{x})—, —N(R^{x})S(O)ₙ—, C₃-C₁₅ carbocyclcyl, 3- to 15-membered heterocyclcyl, C₆-C₁₄ aryl, or 5- to 20-membered heteroaryl;
   wherein,
      each C₃-C₁₅ carbocyclcyl, 3- to 15-membered heterocyclcyl, C₆-C₁₄ aryl, or 5- to 20-membered heteroaryl is optionally substituted with one or more deutero, halo, (C₁-C₆)alkyl, (C₁-C₆)haloalkyl, oxo (C═O), —O(C₁-C₆)alkyl, —O(C₁-C₆)haloalkyl, —O(C₁-C₆)cycloalkyl, —N(R^{xx})(R^{yy}), —C(O)R^{z}, —C(O)OR^{z}, —OC(O)R^{z}, —OC(O)OR^{z}, —OC(O)N(R^{xx})(R^{yy}), —N(R^{xx})C(O)OR^{z}, —N(R^{xx})C(O)N((R^{xx})(R^{yy}), —C(O)N(R^{xx})(R^{yy}), —N(R^{xx})C(O)R^{z}, —N(R^{xx})(R^{yy}); —S(O)ₙR^{z}, —S(O)ₙOR^{z}, —OS(O)ₙR^{z}, —OS(O)ₙOR^{z}, —OS(O)ₙN(R^{xx})(R^{yy}), —N(R^{xx})S(O)ₙOR^{z}, —N(R^{xx})S(O)ₙN(R^{xx})(R^{yy}), —S(O)ₙN(R^{xx})(R^{yy}), or —N(R^{xx})S(O)ₙR^{z};
      each R^{x}, R^{xx}, R^{y}, R^{yy}, and R^{z} are independently H, (C₁-C₆)alkyl, (C₁-C₆)haloalkyl, or (C₁-C₆)cycloalkyl; and
   each n is independently 0-2.

In some embodiments of a compound of formula (I), such as a compound of formula (II), (IV), or (V), or any variation or embodiment thereof, or a stereoisomer or tautomer thereof, or a pharmaceutically acceptable salt of any of the foregoing, wherein independently for each L, L¹, L², Y², Y³, R¹, R², R^{k}, R^{m}, Rⁿ, X¹, X², X³, X⁴, X⁵, and Q¹:
Y² and Y³ are each C, or
one of Y² and Y³ is N and the other of Y² and Y³ is C;
X¹ and X² are each independently H, C₁₋₆alkyl, or C₁₋₆alkoxy;
X³ and X⁴ are each independently H, halo, C₁₋₃alkyl, C₁₋₃alkoxy, or 5-10 membered heteroaryl, wherein the C₁₋₃alkyl of X³ and X⁴ is optionally substituted with one of more halo;
X⁵ is H, C₁₋₃alkyl, C₁₋₃alkoxy, or C₃₋₆cycloalkyl;
either
(1) L¹ is absent; and
Q¹ is selected from (i) to (iv):
(i) phenyl, wherein the phenyl of Q¹ is substituted with one or more halo, C₁₋₃alkyl, C₂₋₃alkenyl, —NH₂, —NH—C(O)—(C₁₋₃alkyl), —NH—C(O)-(3-15 membered heterocyclyl), C₃₋₆cycloalkyl, or 5-10 membered heteroaryl, wherein
   the C₁₋₃alkyl is optionally substituted with one or more halo, —NH—C(O)—NH(C₁₋₃alkyl), —NH—C(O)—C₁₋₃alkyl, or —NH—C(O)—C₁₋₃alkoxy,
   the C₃₋₆cycloalkyl is optionally substituted with one or more halo or C₁₋₃alkyl, and
   the 5-10 membered heteroaryl is optionally substituted with one or more C₁₋₃alkyl,
(ii) 3-10 membered heterocyclyl, wherein the 3-10 membered heterocyclyl of Q¹ is optionally substituted with one or more oxo, or C₁₋₃alkyl,
(iii) 5-10 membered heteroaryl, wherein the 5-10 membered heteroaryl of Q¹ is optionally substituted with one or more halo, C₁₋₃alkyl, C₂₋₃alkenyl, C₁₋₃alkoxy, —NH₂, or C₃₋₆cycloalkyl, wherein,
   the C₁₋₃alkyl is optionally substituted with one or more halo, and
   the C₃₋₆cycloalkyl is optionally substituted with one or more halo or C₁₋₃alkyl, and
(iv) C₃₋₆cycloalkyl;
or
(2) L¹ is —CH₂—; and
Q¹ is C₃₋₆cycloalkyl;
L² is —C(O)— or —S(O)₂—
R¹ is H or C₁₋₃alkyl;
R^{k} is H, halo, —OH, —NH₂, or —NH—C(O)C₁₋₃alkyl;
R^{m} is H, —OH, or C₁₋₃alkyl;
Rⁿ is H, C₁₋₃alkyl, or C₃₋₆cycloalkyl or Rⁿ taken together with the carbon atom to which it is attached forms C₃₋₅ cycloalkyl;
or R^{k} is taken together with either R^{m} or Rⁿ, and the atoms to which they are attached, to form cyclopropyl; and
R² is selected from (i) to (vii):
(i) C₁₋₃alkyl, wherein the C₁₋₃alkyl of R² is optionally substituted with one or more R^{a}, wherein R^{a} is:
   (a) —OH,
   (b) cyano,
   (c) C₂₋₃alkynyl,
   (d) C₆₋₁₀aryl, wherein the C₆₋₁₀aryl of R^{a} is optionally substituted with one or more halo, cyano, C₁₋₃alkoxy, or —NH—C(O)—C₁₋₃alkyl,
   (e) 3-10 membered heterocyclyl, wherein the 3-10 membered heterocyclyl of R^{a} is optionally substituted with one or more R^{c}, wherein
      R^{c} is halo, oxo, C₁₋₃alkyl, C₁₋₃alkoxy, —C(O)—C₁₋₃alkyl, or —C(O)—C₁₋₃alkoxy, wherein
         the C₁₋₃alkyl of R^{c} is optionally substituted with one or more halo or C₂₋₃alkynyl, and
         the —C(O)—C₁₋₃alkoxy of R^{c} is optionally substituted with one or more halo,
   (f) —N(R^{c})(R^{d}), wherein R^{c} and R^{d} of —N(R^{c})(R^{d}) are, independently of each other, H, C₁₋₃alkyl, —C(O)—C₁₋₃alkyl, —C(O)—C₁₋₃alkoxy, —C(O)—NH₂, —C(O)—NH(C₁₋₃alkyl), —C(O)—N(C₁₋₃alkyl)₂, —C(O)-(3-10 membered heterocyclyl), —CH₂—C(O)—NH₂, 3-10 membered heterocyclyl, or 5-10 membered heteroaryl, wherein
      the C₁₋₃alkyl of R^{c} or R^{d} is optionally substituted with one or more —C(O)—NH₂,
      the —C(O)—C₁₋₃alkyl of R^{c} or R^{d} is optionally substituted with one or more halo,
      the 3-10 membered heterocyclyl and the 5-10 membered heteroaryl of R^{c} or R^{d} are independently optionally substituted with one or more C₁₋₃alkyl,
      the —C(O)-(3-10 membered heterocyclyl) of R^{c} or R^{d} is optionally substituted with one or more halo, —C(O)—C₁₋₃alkoxy, or C₁₋₃alkyl, wherein the C₁₋₃alkyl is optionally substituted with one or more halo, C₁₋₃alkoxy, or C₃₋₆cycloalkyl, and
      the C₁₋₃alkyl of the —C(O)—N(C₁₋₃alkyl)₂ of R^{c} or R^{d} are, independently of each other, optionally substituted with one or more halo or C₆₋₁₀aryl,
   (g) —O—R^{e}, wherein R^{e} is C₁₋₃alkyl, C₆₋₁₀aryl, —C(O)-(3-10 membered heterocyclyl), —C(O)—N—(C₁₋₃alkyl)₂, or 5-10 membered heteroaryl, wherein
      the C₁₋₃alkyl of R^{e} is optionally substituted with one or more C₁₋₃alkoxy, wherein the C₁₋₃alkoxy is optionally substituted with one or more C₂₋₃alkynyl,
      the C₆₋₁₀aryl of R^{e} is optionally substituted with one or more C₁₋₃alkyl, and
      the —C(O)-(3-10 membered heterocyclyl) of R^{e} is optionally substituted with one or more C₁₋₃alkyl, C₁₋₃alkoxy, or —C(O)—C₁₋₃alkoxy, wherein the C₁₋₆alkyl is optionally substituted with one or more halo, C₁₋₃alkoxy, or C₃₋₆cycloalkyl,
   (h) —C(O)—R^{e}, wherein R^{e} is —NH₂, —OH, or 3-10 membered heterocyclyl, or
   (i) —S(O)₂—R^{f}, wherein R^{f} is C₁₋₆alkyl or 3-10 membered heterocyclyl,
(ii) C₃₋₆cycloalkyl, wherein the C₃₋₆cycloalkyl of R² is optionally substituted with one or more R^{q}, wherein R^{q} is 5-10 membered heteroaryl or C₆₋₁₀aryl, wherein the C₆₋₁₀aryl of R^{q} is optionally substituted with one or more C₁₋₃alkoxy,
(iii) 3-10 membered heterocyclyl, wherein the 3-10 membered heterocyclyl of R² is optionally substituted with one or more halo, oxo, C₁₋₃alkyl, —C(O)—C₁₋₃alkyl, or 5-10 membered heteroaryl,
(iv) 5-10 membered heteroaryl or —(C₁₋₃alkyl)(5-10 membered heteroaryl), wherein the C₁₋₃ alkyl is optionally substituted with one or more or more —OH, halo, —NH₂, —NH(C₁₋₃alkyl), —N(C₁₋₃alkyl)₂, and wherein the 5-10 membered heteroaryl of the 5-10 membered heteroaryl or —(C₁₋₃alkyl)(5-10 membered heteroaryl), is optionally substituted with one or more R^{s}, wherein
   R^{s} is halo, C₁₋₃alkyl, C₁₋₃alkoxy, —NH₂, —NH(C₁₋₃alkyl), —N(C₁₋₃alkyl)₂, —NH—C(O)—C₁₋₃alkyl, C₆₋₁₀aryl, C₃₋₆cycloalkyl, 3-10 membered heterocyclyl, 5-10 membered heteroaryl, or —C(O)—C₁₋₃alkoxy, wherein
      the C₁₋₃alkyl of R^{s} is optionally substituted with one or more halo, C₁₋₃alkoxy, —NH₂, —NH(C₁₋₃alkyl), —N(C₁₋₃alkyl)₂, —NH—C(O)C₁₋₃alkyl, or —NH—C(O)—C₁₋₃alkoxy, and
      the 3-10-membered heterocyclyl of R^{s} is optionally substituted with one or more halo or —C(O)—C₁₋₃alkoxy,
(v) —N(R^{g})(R^{h}), wherein R⁹ and R^{h} are independently H or C₁₋₃alkyl,
(vi) —C(O)—R¹, wherein Ri is C₃₋₆cycloalkyl, —NH(C₁₋₃alkyl), —N(C₁₋₃alkyl)₂, or —NH(5-10 membered heteroaryl), and
(vii) C₆₋₁₀aryl, wherein the C₆₋₁₀aryl of R² is optionally substituted with one or more 5-10 membered heteroaryl or —O—R^{p}, wherein R^{p} is 3-10 membered heterocyclyl, wherein the 3-10 membered heterocyclyl of R^{p} is optionally substituted with one or more —C(O)—C₁₋₃alkyl;
L is selected from the group consisting of a bond or (C₁-C₂₀)alkyl, wherein
   the (C₁-C₂₀)alkyl is optionally substituted with one or more deutero, halo, (C₁-C₃)alkyl, (C₁-C₃)haloalkyl, oxo (C═O), —O(C₁-C₃)alkyl, —O(C₁-C₃)haloalkyl, —O(C₁-C₃)cycloalkyl, —N(R^{x})(R^{y})—, C₃-C₁₀ carbocyclcyl, 3- to 10-membered heterocyclcyl, C₆-C₁₀ aryl, or 5- to 10-membered heteroaryl; and
   one or more of the C atoms in the (C₁-C₂₀)alkyl is optionally replaced with one or more —C(R^{x})═C(R^{x})—, —C≡C—, —O—, —C(O)—, —C(O)O—, —OC(O)—, —OC(O)O—, —OC(O)N(R^{x})—, —N(R^{x})C(O)O—, —N(R^{x})C(O)N(R^{y})—, —C(O)N(R^{x})—, —N(R^{x})C(O)—, —N(R^{x})—, —S(O)ₙ—, —S(O)ₙO—, —OS(O)ₙ—, —OS(O)ₙO—, —OS(O)ₙN(R^{x})—, —N(R^{x})S(O)ₙO—, —N(R^{x})S(O)ₙN(R^{y})—, —S(O)ₙN(R^{x})—, —N(R^{x})S(O)ₙ—, C₃-C₁₅ carbocyclcyl, 3- to 10-membered heterocyclcyl, C₆-C₁₀ aryl, or 5- to 10-membered heteroaryl;
   wherein,
      each C₃-C₁₀ carbocyclcyl, 3- to 10-membered heterocyclcyl, C₆-C₁₀ aryl, or 5- to 10-membered heteroaryl is optionally substituted with one or more deutero, halo, (C₁-C₃)alkyl, (C₁-C₃)haloalkyl, oxo (C═O), —O(C₁-C₃)alkyl, —O(C₁-C₃)haloalkyl, —O(C₁-C₃)cycloalkyl, —N(R^{xx})(R^{yy}), —C(O)R^{z}, —C(O)OR^{z}, —OC(O)R^{z}, —OC(O)OR^{z}, —OC(O)N(R^{xx})(R^{yy}), —N(R^{xx})C(O)OR^{z}, —N(R^{xx})C(O)N((R^{xx})(R^{yy}), —C(O)N(R^{xx})(R^{yy}), —N(R^{xx})C(O)R^{z}, —N(R^{xx})(R^{yy}); —S(O)ₙR^{z}, —S(O)ₙOR^{z}, —OS(O)ₙR^{z}, —OS(O)ₙOR^{z}, —OS(O)ₙN(R^{xx})(R^{yy}), —N(R^{xx})S(O)ₙOR^{z}, —N(R^{xx})S(O)ₙN(R^{xx})(R^{yy}), —S(O)ₙN(R^{xx})(R^{yy}), or —N(R^{xx})S(O)ₙR^{z};
      each R^{x}, R^{xx}, R^{y}, R^{yy}, and R^{z} are independently H, (C₁-C₃)alkyl, (C₁-C₃)haloalkyl, or (C₁-C₃)cycloalkyl; and
   each n is independently 0-2.

In some embodiments of a compound of formula (I), such as a compound of formula (II), (IV), or (V), or any variation or embodiment thereof, or a stereoisomer or tautomer thereof, or a pharmaceutically acceptable salt of any of the foregoing, wherein independently for each L, L¹, L², Y², Y³, R¹, R², R^{k}, R^{m}, Rⁿ, X¹, X², X³, X⁴, X⁵, and Q¹:
one of Y² and Y³ is N and the other of Y² and Y³ is C;
X¹ and X² are each independently H;
X³ and X⁴ are each independently H, or halo;
X⁵ is C₃₋₆cycloalkyl;
L¹ is absent;
Q¹ is phenyl;
L² is —C(O)—;
R¹ is H;
R^{k} is halo;
R^{m} is H;
Rⁿ is H;
R² is selected from (i) to (ii):
(i) C₁₋₃alkyl, wherein the C₁₋₃alkyl of R² is optionally substituted with one or more R^{a}, wherein R^{a} is 3-10 membered heterocyclyl, wherein the 3-10 membered heterocyclyl of R^{a} is optionally substituted with one or more R^{c}, wherein R^{c} is oxo, or C₁₋₃alkyl, or
(ii) 5-10 membered heteroaryl or —(C₁₋₃alkyl)(5-10 membered heteroaryl);
L is (C₁-C₂₀)alkyl, wherein
   one or more of the C atoms in the (C₁-C₂₀)alkyl is optionally replaced with one or more —O—, —N(R^{x})—, or 3- to 10-membered heterocyclcyl;
   wherein,
      each 3- to 10-membered heterocyclcyl is optionally substituted with one or more (C₁-C₃)alkyl, or oxo (C═O); and
      each R^{x} is independently H.

In some embodiments of a compound of formula (I), such as a compound of formula (II), or (IV), or any variation or embodiment thereof, or a stereoisomer or tautomer thereof, or a pharmaceutically acceptable salt of any of the foregoing, wherein independently for each L, L¹, L², Y², Y³, R¹, R², R^{k}, R^{m}, Rⁿ, X¹, X², X³, X⁴, X⁵, and Q¹:
one of Y² and Y³ is N and the other of Y² and Y³ is C;
X¹ and X² are each independently H;
X³ and X⁴ are each independently H, or halo;
X⁵ is C₃₋₆cycloalkyl;
L¹ is absent;
Q¹ is phenyl;
L² is —C(O)—;
R¹ is H;
R^{k} is halo;
R^{m} is H;
Rⁿ is H;
R² is —(C₁₋₃alkyl)(5-10 membered heteroaryl);
L is (C₁-C₂₀)alkyl, wherein
   one or more of the C atoms in the (C₁-C₂₀)alkyl is optionally replaced with one or more —O—, —N(R^{x})—, or 3- to 10-membered heterocyclcyl;
   wherein,
      each 3- to 10-membered heterocyclcyl is optionally substituted with one or more (C₁-C₃)alkyl, or oxo (C═O); and
      each R^{x} is independently H.

In some embodiments of a compound of formula (I), such as a compound of formula (III), or (IV), or any variation or embodiment thereof, or a stereoisomer or tautomer thereof, or a pharmaceutically acceptable salt of any of the foregoing, wherein independently for each L, L¹, L², Y², Y³, R², X¹, X², X³, X⁴, X⁵, and Q¹:
Y² and Y³ are each C, or
one of Y² and Y³ is N and the other of Y² and Y³ is C;
X¹ and X² are each independently H, C₁₋₆alkyl, or C₁₋₆alkoxy;
X³ and X⁴ are each independently H, halo, C₁₋₆alkyl, C₁₋₆alkoxy, or 5-20 membered heteroaryl, wherein the C₁₋₆alkyl of X³ and X⁴ is optionally substituted with one of more halo;
X⁵ is H, C₁₋₆alkyl, C₁₋₆alkoxy, or C₃₋₁₀cycloalkyl;
either
(1) L¹ is absent; and
Q¹ is selected from (i) to (iv):
(i) phenyl, wherein the phenyl of Q¹ is substituted with one or more halo, C₁₋₆alkyl, C₂₋₆alkenyl, —NH₂, —NH—C(O)—(C₁₋₆alkyl), —NH—C(O)-(3-15 membered heterocyclyl), C₃₋₁₀cycloalkyl, or 5-20 membered heteroaryl, wherein
   the C₁₋₆alkyl is optionally substituted with one or more halo, —NH—C(O)—NH(C₁₋₆alkyl), —NH—C(O)—C₁₋₆alkyl, or —NH—C(O)—C₁₋₆alkoxy,
   the C₃₋₁₀cycloalkyl is optionally substituted with one or more halo or C₁₋₆alkyl, and
   the 5-20 membered heteroaryl is optionally substituted with one or more C₁₋₆alkyl,
(ii) 3-15 membered heterocyclyl, wherein the 3-15 membered heterocyclyl of Q¹ is optionally substituted with one or more oxo, or C₁₋₆alkyl,
(iii) 5-20 membered heteroaryl, wherein the 5-20 membered heteroaryl of Q¹ is optionally substituted with one or more halo, C₁₋₆alkyl, C₂₋₆alkenyl, C₁₋₆alkoxy, —NH₂, or C₃₋₁₀cycloalkyl, wherein,
   the C₁₋₆alkyl is optionally substituted with one or more halo, and
   the C₃₋₁₀cycloalkyl is optionally substituted with one or more halo or C₁₋₆alkyl, and
(iv) C₃₋₁₀cycloalkyl;
or
(2) L¹ is —CH₂—; and
Q¹ is C₃₋₁₀cycloalkyl;
L² is —C(O)— or —S(O)₂—
R² is selected from (i) to (vii):
(i) C₁₋₆alkyl, wherein the C₁₋₆alkyl of R² is optionally substituted with one or more R^{a}, wherein R^{a} is:
   (a) —OH,
   (b) cyano,
   (c) C₂₋₆alkynyl,
   (d) C₆₋₂₀aryl, wherein the C₆₋₂₀aryl of R^{a} is optionally substituted with one or more halo, cyano, C₁₋₆alkoxy, or —NH—C(O)—C₁₋₆alkyl,
   (e) 3-15 membered heterocyclyl, wherein the 3-15 membered heterocyclyl of R^{a} is optionally substituted with one or more R^{c}, wherein
      R^{c} is halo, oxo, C₁₋₆alkyl, C₁₋₆alkoxy, —C(O)—C₁₋₆alkyl, or —C(O)—C₁₋₆alkoxy, wherein
         the C₁₋₆alkyl of R^{c} is optionally substituted with one or more halo or C₂₋₆alkynyl, and
         the —C(O)—C₁₋₆alkoxy of R^{c} is optionally substituted with one or more halo,
   (f) —N(R^{c})(R^{d}), wherein R^{c} and R^{d} of —N(R^{c})(R^{d}) are, independently of each other, H, C₁₋₆alkyl, —C(O)—C₁₋₆alkyl, —C(O)—C₁₋₆alkoxy, —C(O)—NH₂, —C(O)—NH(C₁₋₆alkyl), —C(O)—N(C₁₋₆alkyl)₂, —C(O)-(3-15 membered heterocyclyl), —CH₂—C(O)—NH₂, 3-15 membered heterocyclyl, or 5-20 membered heteroaryl, wherein
      the C₁₋₆alkyl of R^{c} or R^{d} is optionally substituted with one or more —C(O)—NH₂,
      the —C(O)—C₁₋₆alkyl of R^{c} or R^{d} is optionally substituted with one or more halo,
      the 3-15 membered heterocyclyl and the 5-20 membered heteroaryl of R^{c} or R^{d} are independently optionally substituted with one or more C₁₋₆alkyl,
      the —C(O)-(3-15 membered heterocyclyl) of R^{c} or R^{d} is optionally substituted with one or more halo, —C(O)—C₁₋₆alkoxy, or C₁₋₆alkyl, wherein the C₁₋₆alkyl is optionally substituted with one or more halo, C₁₋₆alkoxy, or C₃₋₁₀cycloalkyl, and
      the C₁₋₆alkyl of the —C(O)—N(C₁₋₆alkyl)₂ of R^{c} or R^{d} are, independently of each other, optionally substituted with one or more halo or C₆₋₂₀aryl,
   (g) —O—R^{e}, wherein R^{e} is C₁₋₆alkyl, C₆₋₂₀aryl, —C(O)-(3-15 membered heterocyclyl), —C(O)—N—(C₁₋₆alkyl)₂, or 5-20 membered heteroaryl, wherein
      the C₁₋₆alkyl of R^{e} is optionally substituted with one or more C₁₋₆alkoxy, wherein the C₁₋₆alkoxy is optionally substituted with one or more C₂₋₆alkynyl,
      the C₆₋₂₀aryl of R^{e} is optionally substituted with one or more C₁₋₆alkyl, and
      the —C(O)-(3-15 membered heterocyclyl) of R^{e} is optionally substituted with one or more C₁₋₆alkyl, C₁₋₆alkoxy, or —C(O)—C₁₋₆alkoxy, wherein the C₁₋₆alkyl is optionally substituted with one or more halo, C₁₋₆alkoxy, or C₃₋₁₀cycloalkyl,
   (h) —C(O)—R^{e}, wherein R^{e} is —NH₂, —OH, or 3-15 membered heterocyclyl, or
   (i) —S(O)₂—R^{f}, wherein R^{f} is C₁₋₆alkyl or 3-15 membered heterocyclyl,
(ii) C₃₋₁₀cycloalkyl, wherein the C₃₋₁₀cycloalkyl of R² is optionally substituted with one or more R^{q}, wherein R^{q} is 5-20 membered heteroaryl or C₆₋₂₀aryl, wherein the C₆₋₂₀aryl of R^{q} is optionally substituted with one or more C₁₋₆alkoxy,
(iii) 3-15 membered heterocyclyl, wherein the 3-15 membered heterocyclyl of R² is optionally substituted with one or more halo, oxo, C₁₋₆alkyl, —C(O)—C₁₋₆alkyl, or 5-20 membered heteroaryl,
(iv) 5-20 membered heteroaryl or —(C₁₋₄alkyl)(5-20 membered heteroaryl), wherein the C₁₋₄ alkyl is optionally substituted with one or more or more —OH, halo, —NH₂, —NH(C₁₋₆alkyl), —N(C₁₋₆alkyl)₂, and wherein the 5-20 membered heteroaryl of the 5-20 membered heteroaryl or —(C₁₋₄alkyl)(5-20 membered heteroaryl), is optionally substituted with one or more R^{s}, wherein
   R^{s} is halo, C₁₋₆alkyl, C₁₋₆alkoxy, —NH₂, —NH(C₁₋₆alkyl), —N(C₁₋₆alkyl)₂, —NH—C(O)—C₁₋₆alkyl, C₆₋₂₀aryl, C₃₋₁₀cycloalkyl, 3-15 membered heterocyclyl, 5-20 membered heteroaryl, or —C(O)—C₁₋₆alkoxy, wherein
      the C₁₋₆alkyl of R^{s} is optionally substituted with one or more halo, C₁₋₆alkoxy, —NH₂, —NH(C₁₋₆alkyl), —N(C₁₋₆alkyl)₂, —NH—C(O)C₁₋₆alkyl, or —NH—C(O)—C₁₋₆alkoxy, and
      the 3-15-membered heterocyclyl of R^{s} is optionally substituted with one or more halo or —C(O)—C₁₋₆alkoxy,
(v) —N(R^{g})(R^{h}), wherein R^{g} and R^{h} are independently H or C₁₋₆alkyl,
(vi) —C(O)—R^{j}, wherein Ri is C₃₋₁₀cycloalkyl, —NH(C₁₋₆alkyl), —N(C₁₋₆alkyl)₂, or —NH(5-20 membered heteroaryl), and
(vii) C₆₋₂₀aryl, wherein the C₆₋₂₀aryl of R² is optionally substituted with one or more 5-20 membered heteroaryl or —O—R^{p}, wherein R^{p} is 3-15 membered heterocyclyl, wherein the 3-15 membered heterocyclyl of R^{p} is optionally substituted with one or more —C(O)—C₁₋₆alkyl;
L is selected from the group consisting of a bond or (C₁-C₅₀)alkyl, wherein
   the (C₁-C₅₀)alkyl is optionally substituted with one or more deutero, halo, (C₁-C₆)alkyl, (C₁-C₆)haloalkyl, oxo (C═O), —O(C₁-C₆)alkyl, —O(C₁-C₆)haloalkyl, —O(C₁-C₆)cycloalkyl, —N(R^{x})(R^{y})—, C₃-C₁₅ carbocyclcyl, 3- to 15-membered heterocyclcyl, C₆-C₁₄ aryl, or 5- to 20-membered
   heteroaryl; and one or more of the C atoms in the (C₁-C₅₀)alkyl is optionally replaced with one or more —C(R^{x})═C(R^{x})—, —C≡C—, —O—, —C(O)—, —C(O)O—, —OC(O)—, —OC(O)O—, —OC(O)N(R^{x})—, —N(R^{x})C(O)O—, —N(R^{x})C(O)N(R^{y})—, —C(O)N(R^{x})—, —N(R^{x})C(O)—, —N(R^{x})—, —S(O)ₙ—, —S(O)ₙO—, —OS(O)ₙ—, —OS(O)ₙO—, —OS(O)ₙN(R^{x})—, —N(R^{x})S(O)ₙO—, —N(R^{x})S(O)ₙN(R^{y})—, —S(O)ₙN(R^{x})—, —N(R^{x})S(O)ₙ—, C₃-C₁₅ carbocyclcyl, 3- to 15-membered heterocyclcyl, C₆-C₁₄ aryl, or 5- to 20-membered heteroaryl;
   wherein,
      each C₃-C₁₅ carbocyclcyl, 3- to 15-membered heterocyclcyl, C₆-C₁₄ aryl, or 5- to 20-membered heteroaryl is optionally substituted with one or more deutero, halo, (C₁-C₆)alkyl, (C₁-C₆)haloalkyl, oxo (C═O), —O(C₁-C₆)alkyl, —O(C₁-C₆)haloalkyl, —O(C₁-C₆)cycloalkyl, —N(R^{xx})(R^{yy}), —C(O)R^{z}, —C(O)OR^{z}, —OC(O)R^{z}, —OC(O)OR^{z}, —OC(O)N(R^{x})(R^{y}), —N(R^{xx})C(O)OR^{z}, —N(R^{xx})C(O)N((R^{xx})(R^{yy}), —C(O)N(R^{xx})(R^{yy}), —N(R^{xx})C(O)R^{z}, —N(R^{xx})(R^{yy}); —S(O)ₙR^{z}, —S(O)ₙOR^{z}, —OS(O)R^{z}, —OS(O)ₙOR^{z}, —OS(O)ₙN(R^{xx})(R^{yy}), —N(R^{xx})S(O)ₙOR^{z}, —N(R^{xx})S(O)ₙN(R^{xx})(R^{yy}), —S(O)ₙN(R^{xx})(R^{yy}), or —N(R^{xx})S(O)ₙR^{z};
      each R^{x}, R^{xx}, R^{y}, R^{yy}, and R^{z} are independently H, (C₁-C₆)alkyl, (C₁-C₆)haloalkyl, or (C₁-C₆)cycloalkyl; and
each n is independently 0-2.

In some embodiments of a compound of formula (I), such as a compound of formula (III), or (IV) or any variation or embodiment thereof, or a stereoisomer or tautomer thereof, or a pharmaceutically acceptable salt of any of the foregoing, wherein independently for each L, L¹, L², Y², Y³, R², X¹, X², X³, X⁴, X⁵, and Q¹:
Y² and Y³ are each C, or
one of Y² and Y³ is N and the other of Y² and Y³ is C;
X¹ and X² are each independently H, C₁₋₆alkyl, or C₁₋₆alkoxy;
X³ and X⁴ are each independently H, halo, C₁₋₃alkyl, C₁₋₃alkoxy, or 5-10 membered heteroaryl, wherein the C₁₋₃alkyl of X³ and X⁴ is optionally substituted with one of more halo;
X⁵ is H, C₁₋₃alkyl, C₁₋₃alkoxy, or C₃₋₆cycloalkyl;
either
(1) L¹ is absent; and
Q¹ is selected from (i) to (iv):
(i) phenyl, wherein the phenyl of Q¹ is substituted with one or more halo, C₁₋₃alkyl, C₂₋₃alkenyl, —NH₂, —NH—C(O)—(C₁₋₃alkyl), —NH—C(O)-(3-15 membered heterocyclyl), C₃₋₆cycloalkyl, or 5-10 membered heteroaryl, wherein
   the C₁₋₃alkyl is optionally substituted with one or more halo, —NH—C(O)—NH(C₁₋₃alkyl), —NH—C(O)—C₁₋₃alkyl, or —NH—C(O)—C₁₋₃alkoxy,
   the C₃₋₆cycloalkyl is optionally substituted with one or more halo or C₁₋₃alkyl, and
   the 5-10 membered heteroaryl is optionally substituted with one or more C₁₋₃alkyl,
(ii) 3-10 membered heterocyclyl, wherein the 3-10 membered heterocyclyl of Q¹ is optionally substituted with one or more oxo, or C₁₋₃alkyl,
(iii) 5-10 membered heteroaryl, wherein the 5-10 membered heteroaryl of Q¹ is optionally substituted with one or more halo, C₁₋₃alkyl, C₂₋₃alkenyl, C₁₋₃alkoxy, —NH₂, or C₃₋₆cycloalkyl, wherein,
   the C₁₋₃alkyl is optionally substituted with one or more halo, and
   the C₃₋₆cycloalkyl is optionally substituted with one or more halo or C₁₋₃alkyl, and
(iv) C₃₋₆cycloalkyl;
or
(2) L¹ is —CH₂—; and
Q¹ is C₃₋₆cycloalkyl;
L² is —C(O)— or —S(O)₂—; and
R² is selected from (i) to (vii):
(i) C₁₋₃alkyl, wherein the C₁₋₃alkyl of R² is optionally substituted with one or more R^{a}, wherein R^{a} is:
   (a) —OH,
   (b) cyano,
   (c) C₂₋₃alkynyl,
   (d) C₆₋₁₀aryl, wherein the C₆₋₁₀aryl of R^{a} is optionally substituted with one or more halo, cyano, C₁₋₃alkoxy, or —NH—C(O)—C₁₋₃alkyl,
   (e) 3-10 membered heterocyclyl, wherein the 3-10 membered heterocyclyl of R^{a} is optionally substituted with one or more R^{c}, wherein
      R^{c} is halo, oxo, C₁₋₃alkyl, C₁₋₃alkoxy, —C(O)—C₁₋₃alkyl, or —C(O)—C₁₋₃alkoxy, wherein
         the C₁₋₃alkyl of R^{c} is optionally substituted with one or more halo or C₂₋₃alkynyl, and
         the —C(O)—C₁₋₃alkoxy of R^{c} is optionally substituted with one or more halo,
   (f) —N(R^{c})(R^{d}), wherein R^{c} and R^{d} of —N(R^{c})(R^{d}) are, independently of each other, H, C₁₋₃alkyl, —C(O)—C₁₋₃alkyl, —C(O)—C₁₋₃alkoxy, —C(O)—NH₂, —C(O)—NH(C₁₋₃alkyl), —C(O)—N(C₁₋₃alkyl)₂, —C(O)-(3-10 membered heterocyclyl), —CH₂—C(O)—NH₂, 3-10 membered heterocyclyl, or 5-10 membered heteroaryl, wherein
      the C₁₋₃alkyl of R^{c} or R^{d} is optionally substituted with one or more —C(O)—NH₂,
      the —C(O)—C₁₋₃alkyl of R^{c} or R^{d} is optionally substituted with one or more halo,
      the 3-10 membered heterocyclyl and the 5-10 membered heteroaryl of R^{c} or R^{d} are independently optionally substituted with one or more C₁₋₃alkyl,
      the —C(O)-(3-10 membered heterocyclyl) of R^{c} or R^{d} is optionally substituted with one or more halo, —C(O)—C₁₋₃alkoxy, or C₁₋₃alkyl, wherein the C₁₋₃alkyl is optionally substituted with one or more halo, C₁₋₃alkoxy, or C₃₋₆cycloalkyl, and
      the C₁₋₃alkyl of the —C(O)—N(C₁₋₃alkyl)₂ of R^{c} or R^{d} are, independently of each other, optionally substituted with one or more halo or C₆₋₁₀aryl,
   (g) —O—R^{e}, wherein R^{e} is C₁₋₃alkyl, C₆₋₁₀aryl, —C(O)-(3-10 membered heterocyclyl), —C(O)—N—(C₁₋₃alkyl)₂, or 5-10 membered heteroaryl, wherein
      the C₁₋₃alkyl of R^{e} is optionally substituted with one or more C₁₋₃alkoxy, wherein the C₁₋₃alkoxy is optionally substituted with one or more C₂₋₃alkynyl,
      the C₆₋₁₀aryl of R^{e} is optionally substituted with one or more C₁₋₃alkyl, and
      the —C(O)-(3-10 membered heterocyclyl) of R^{e} is optionally substituted with one or more C₁₋₃alkyl, C₁₋₃alkoxy, or —C(O)—C₁₋₃alkoxy, wherein the C₁₋₆alkyl is optionally substituted with one or more halo, C₁₋₃alkoxy, or C₃₋₆cycloalkyl,
   (h) —C(O)—R^{e}, wherein R^{e} is —NH₂, —OH, or 3-10 membered heterocyclyl, or
   (i) —S(O)₂—R^{f}, wherein R^{f} is C₁₋₆alkyl or 3-10 membered heterocyclyl,
(ii) C₃₋₆cycloalkyl, wherein the C₃₋₆cycloalkyl of R² is optionally substituted with one or more R^{q}, wherein R^{q} is 5-10 membered heteroaryl or C₆₋₁₀aryl, wherein the C₆₋₁₀aryl of R^{q} is optionally substituted with one or more C₁₋₃alkoxy,
(iii) 3-10 membered heterocyclyl, wherein the 3-10 membered heterocyclyl of R² is optionally substituted with one or more halo, oxo, C₁₋₃alkyl, —C(O)—C₁₋₃alkyl, or 5-10 membered heteroaryl,
(iv) 5-10 membered heteroaryl or —(C₁₋₃alkyl)(5-10 membered heteroaryl), wherein the C₁₋₃ alkyl is optionally substituted with one or more or more —OH, halo, —NH₂, —NH(C₁₋₃alkyl), —N(C₁₋₃alkyl)₂, and wherein the 5-10 membered heteroaryl of the 5-10 membered heteroaryl or —(C₁₋₃alkyl)(5-10 membered heteroaryl), is optionally substituted with one or more R^{s}, wherein
   R^{s} is halo, C₁₋₃alkyl, C₁₋₃alkoxy, —NH₂, —NH(C₁₋₃alkyl), —N(C₁₋₃alkyl)₂, —NH—C(O)—C₁₋₃alkyl, C₆₋₁₀aryl, C₃₋₆cycloalkyl, 3-10 membered heterocyclyl, 5-10 membered heteroaryl, or —C(O)—C₁₋₃alkoxy, wherein
      the C₁₋₃alkyl of R^{s} is optionally substituted with one or more halo, C₁₋₃alkoxy, —NH₂, —NH(C₁₋₃alkyl), —N(C₁₋₃alkyl)₂, —NH—C(O)C₁₋₃alkyl, or —NH—C(O)—C₁₋₃alkoxy, and
      the 3-10-membered heterocyclyl of R^{s} is optionally substituted with one or more halo or —C(O)—C₁₋₃alkoxy,
(v) —N(R^{g})(R^{h}), wherein R^{g} and R^{h} are independently H or C₁₋₃alkyl,
(vi) —C(O)—R¹, wherein Ri is C₃₋₆cycloalkyl, —NH(C₁₋₃alkyl), —N(C₁₋₃alkyl)₂, or —NH(5-10 membered heteroaryl), and
(vii) C₆₋₁₀aryl, wherein the C₆₋₁₀aryl of R² is optionally substituted with one or more 5-10 membered heteroaryl or —O—R^{p}, wherein R^{p} is 3-10 membered heterocyclyl, wherein the 3-10 membered heterocyclyl of R^{p} is optionally substituted with one or more —C(O)—C₁₋₃alkyl;
L is selected from the group consisting of a bond or (C₁-C₂₀)alkyl, wherein
   the (C₁-C₂₀)alkyl is optionally substituted with one or more deutero, halo, (C₁-C₃)alkyl, (C₁-C₃)haloalkyl, oxo (C═O), —O(C₁-C₃)alkyl, —O(C₁-C₃)haloalkyl, —O(C₁-C₃)cycloalkyl, —N(R^{x})(R^{y})—, C₃-C₁₀ carbocyclcyl, 3- to 10-membered heterocyclcyl, C₆-C₁₀ aryl, or 5- to 10-membered heteroaryl; and
   one or more of the C atoms in the (C₁-C₂₀)alkyl is optionally replaced with one or more —C(R^{x})═C(R^{x})—, —C≡C—, —O—, —C(O)—, —C(O)O—, —OC(O)—, —OC(O)O—, —OC(O)N(R^{x})—, —N(R^{x})C(O)O—, —N(R^{x})C(O)N(R^{y})—, —C(O)N(R^{x})—, —N(R^{x})C(O)—, —N(R^{x})—, —S(O)ₙ—, —S(O)ₙO—, —OS(O)ₙ—, —OS(O)ₙO—, —OS(O)ₙN(R^{x})—, —N(R^{x})S(O)ₙO—, —N(R^{x})S(O)ₙN(R^{y})—, —S(O)ₙN(R^{x})—, —N(R^{x})S(O)ₙ—, C₃-C₁₅ carbocyclcyl, 3- to 10-membered heterocyclcyl, C₆-C₁₀ aryl, or 5- to 10-membered heteroaryl;
   wherein,
      each C₃-C₁₀ carbocyclcyl, 3- to 10-membered heterocyclcyl, C₆-C₁₀ aryl, or 5- to 10-membered heteroaryl is optionally substituted with one or more deutero, halo, (C₁-C₃)alkyl, (C₁-C₃)haloalkyl, oxo (C═O), —O(C₁-C₃)alkyl, —O(C₁-C₃)haloalkyl, —O(C₁-C₃)cycloalkyl, —N(R^{xx})(R^{yy}), —C(O)R^{z}, —C(O)OR^{z}, —OC(O)R^{z}, —OC(O)OR^{z}, —OC(O)N(R^{xx})(R^{yy}), —N(R^{xx})C(O)OR^{z}, —N(R^{xx})C(O)N((R^{xx})(R^{yy}), —C(O)N(R^{xx})(R^{yy}), —N(R^{xx})C(O)R^{z}, —N(R^{xx})(R^{yy}), —S(O)ₙR^{z}, —S(O)ₙOR^{z}, —OS(O)ₙR^{z}, —OS(O)ₙOR^{z}, —OS(O)ₙN(R^{xx})(R^{yy}), —N(R^{xx})S(O)ₙOR^{z}, —N(R^{xx})S(O)ₙN(R^{xx})(R^{yy}), —S(O)ₙN(R^{xx})(R^{yy}), or —N(R^{xx})S(O)ₙR^{z};
   each R^{x}, R^{xx}, R^{y}, R^{yy}, and R^{z} are independently H, (C₁-C₃)alkyl, (C₁-C₃)haloalkyl, or (C₁-C₃)cycloalkyl; and
each n is independently 0-2.

In some embodiments of a compound of formula (I), such as a compound of formula (III), or (IV), or any variation or embodiment thereof, or a stereoisomer or tautomer thereof, or a pharmaceutically acceptable salt of any of the foregoing, wherein independently for each L, L¹, L², Y², Y³, R², X¹, X², X³, X⁴, X⁵, and Q¹:
one of Y² and Y³ is N and the other of Y² and Y³ is C;
X¹ and X² are each independently H;
X³ and X⁴ are each independently H, or halo;
X⁵ is C₃₋₆cycloalkyl;
L¹ is absent;
Q¹ is phenyl;
L² is —C(O)—;
R² is selected from (i) to (ii):
(i) C₁₋₃alkyl, wherein the C₁₋₃alkyl of R² is optionally substituted with one or more R^{a}, wherein R^{a} is 3-10 membered heterocyclyl, wherein the 3-10 membered heterocyclyl of R^{a} is optionally substituted with one or more R^{c}, wherein R^{c} is oxo, or C₁₋₃alkyl, or
(ii) 5-10 membered heteroaryl or —(C₁₋₃alkyl)(5-10 membered heteroaryl);
L is (C₁-C₂M)alkyl, wherein
   one or more of the C atoms in the (C₁-C₂₀)alkyl is optionally replaced with one or more —O—, —N(R^{x})—, or 3- to 10-membered heterocyclcyl;
   wherein,
      each 3- to 10-membered heterocyclcyl is optionally substituted with one or more (C₁-C₃)alkyl, or oxo (C═O); and
      each R^{x} is independently H.

In some embodiments of a compound of formula (I), such as a compound of formula (II), (II-A), (III), (II-A), (IV), (IV-A), (V), or (V-A), or any variation or embodiment thereof, or a stereoisomer or tautomer thereof, or a pharmaceutically acceptable salt of any of the foregoing, G1—Z1 is selected from the group consisting of

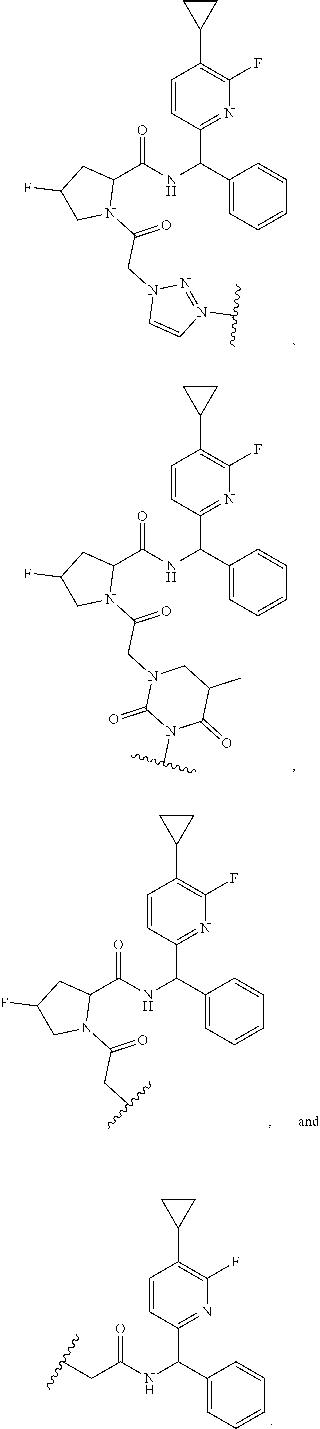

In some embodiments of a compound of formula (I), such as a compound of formula (II), (II-A), (III), (II-A), (IV), (IV-A), (V), or (V-A), or any variation or embodiment thereof, or a stereoisomer or tautomer thereof, or a pharmaceutically acceptable salt ofany of the foregoing, the compound is selected from Table 1.

Compound Names included in Table 1 and for all intermediates and compounds were generated using ChemDraw® Professional software version 17.1.1.0 or Collaborative Drug Discovery Inc. (CDD) CDD Vault update #3.

A Knime workflow was created to retrieve structures from an internal ChemAxon Compound Registry, generate the canonical smiles using RDKit Canon SMILES node, remove the stereochemistry using ChemAxon/Infocom MolConverter node, and name the structure using ChemAxon/Infocom Naming node. The following denotes the version of the Knime Analytics Platform and extensions utilized in the workflow:
Knime Analytics Platform 4.2.2
RDKit Knime Integration 4.0.1.v202006261025 (this extension includes the RDKit Canon SMILES node)
ChemAxon/Infocom Marvin Extensions Feature 4.3.0v202100 (this extension includes the MolConverter node)
ChemAxon/Infocom JChem Extensions Feature 4.3.0v202100 (this extension includes the Naming node)

TABLE 1

| Compound | | |
|---|---|---|
| No. | Structure | IUPAC |
| 1 | 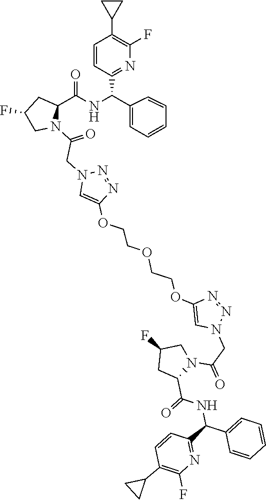 [SMILES: O=C(N[C@@H](c1ccccc1)c1ccc(C2CC2)c(F)n1)[C@@H]1C[C@@H](F)CN1C(=O)Cn1cc(OCCOCCOc2cn(CC(=O)N3C[C@H](F)C[C@H]3C(=O)N[C@@H](c3ccccc3)c3ccc(C4CC4)c(F)n3)nn2)nn1] | (2S,2′S,4R,4′R)-1,1′-(2,2′- (((oxybis(ethane-2,1- diyl))bis(oxy))bis(1H-1,2,3- triazole-4,1-diyl))bis(acetyl))bis(N- ((S)-(5-cyclopropyl-6- fluoropyridin-2-yl)(phenyl)methyl)- 4-fluoropyrrolidine-2-carboxamide) |
| 2 | 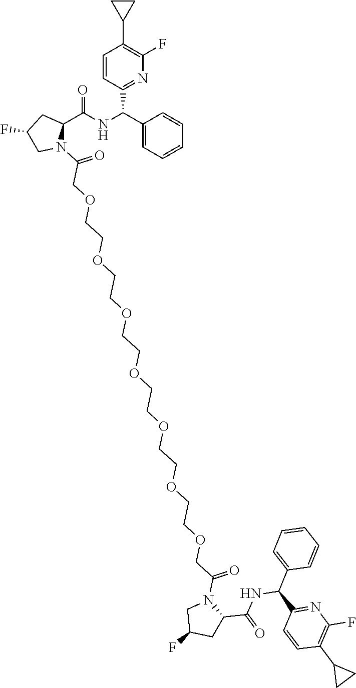 [SMILES: O=C(N[C@@H](c1ccccc1)c1ccc(C2CC2)c(F)n1)[C@@H]1C[C@@H](F)CN1C(=O)COCCOCCOCCOCCOCCOCCOCC(=O)N1C[C@H](F)C[C@H]1C(=O)N[C@@H](c1ccccc1)c1ccc(C2CC2)c(F)n1] | (2S,2′S,4R,4′R)-1,1′- (3,6,9,12,15,18,21- heptaoxatricosanedioyl)bis(N-((S)- (5-cyclopropyl-6-fluoropyridin-2- yl)(phenyl)methyl)-4- fluoropyrrolidine-2-carboxamide) |
| 3 | 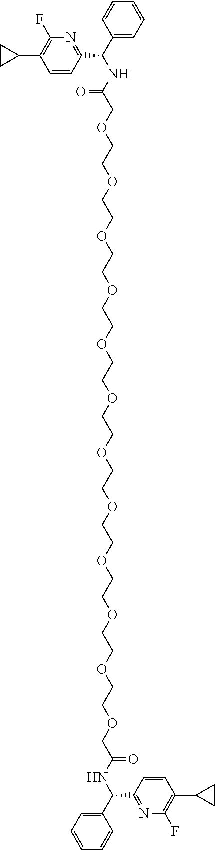 [SMILES: O=C(COCCOCCOCCOCCOCCOCCOCCOCCOCCOCCOCCOCC(=O)N[C@@H](c1ccccc1)c1ccc(C2CC2)c(F)n1)N[C@@H](c1ccccc1)c1ccc(C2CC2)c(F)n1] | N¹,N³⁸-bis((S)-(5-cyclopropyl-6- fluoropyridin-2-yl)(phenyl)methyl)- 3,6,9,12,15,18,21,24,27,30,33,36- dodecaoxaoctatriacontanediamide |
| 4 | 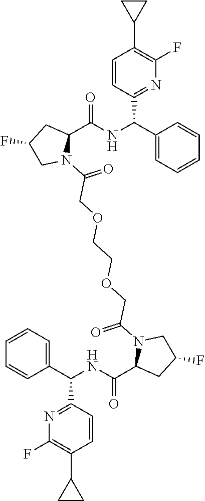 [SMILES: O=C(N[C@@H](c1ccccc1)c1ccc(C2CC2)c(F)n1)[C@@H]1C[C@@H](F)CN1C(=O)COCCOCC(=O)N1C[C@H](F)C[C@H]1C(=O)N[C@@H](c1ccccc1)c1ccc(C2CC2)c(F)n1] | (2S,2′S,4R,4′R)-1,1′-(2,2′-(ethane- 1,2-diylbis(oxy))bis(acetyl))bis(N- ((S)-(5-cyclopropyl-6- fluoropyridin-2-yl)(phenyl)methyl)- 4-fluoropyrrolidine-2-carboxamide) |
| 5 | 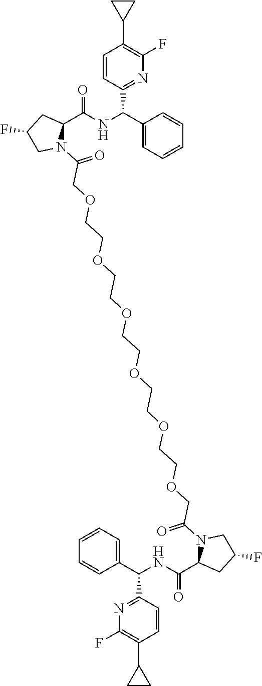 [SMILES: O=C(N[C@@H](c1ccccc1)c1ccc(C2CC2)c(F)n1)[C@@H]1C[C@@H](F)CN1C(=O)COCCOCCOCCOCCOCCOCC(=O)N1C[C@H](F)C[C@H]1C(=O)N[C@@H](c1ccccc1)c1ccc(C2CC2)c(F)n1] | (2S,2′S,4R,4′R)-1,1′- (3,6,9,12,15,18- hexaoxaicosanedioyl)bis(N-((S)-(5- cyclopropyl-6-fluoropyridin-2- yl)(phenyl)methyl)-4- fluoropyrrolidine-2-carboxamide) |
| 6 | 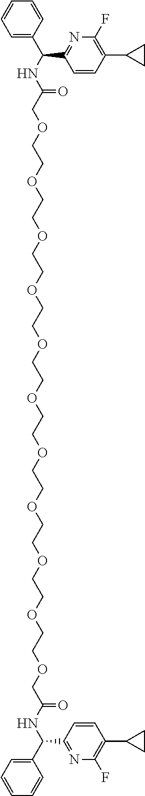 [SMILES: O=C(COCCOCCOCCOCCOCCOCCOCCOCCOCCOCCOCC(=O)N[C@@H](c1ccccc1)c1ccc(C2CC2)c(F)n1)N[C@@H](c1ccccc1)c1ccc(C2CC2)c(F)n1] | N¹,N³⁵-bis((S)-(5-cyclopropyl-6- fluoropyridin-2-yl)(phenyl)methyl)- 3,6,9,12,15,18,21,24,27,30,33- undecaoxapentatriacontanediamide |
| 7 | 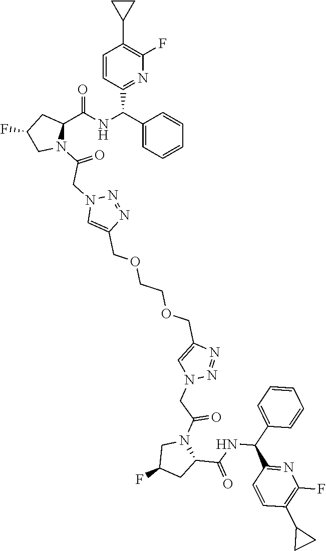 [SMILES: O=C(N[C@@H](c1ccccc1)c1ccc(C2CC2)c(F)n1)[C@@H]1C[C@@H](F)CN1C(=O)Cn1cc(COCCOCc2cn(CC(=O)N3C[C@H](F)C[C@H]3C(=O)N[C@@H](c3ccccc3)c3ccc(C4CC4)c(F)n3)nn2)nn1] | (2S,2′S,4R,4′R)-1,1′-(2,2′-(((ethane- 1,2- diylbis(oxy))bis(methylene))bis(1H- 1,2,3-triazole-4,1- diyl))bis(acetyl))bis(N-((S)-(5- cyclopropyl-6-fluoropyridin-2- yl)(phenyl)methyl)-4- fluoropyrrolidine-2-carboxamide) |
| 8 | 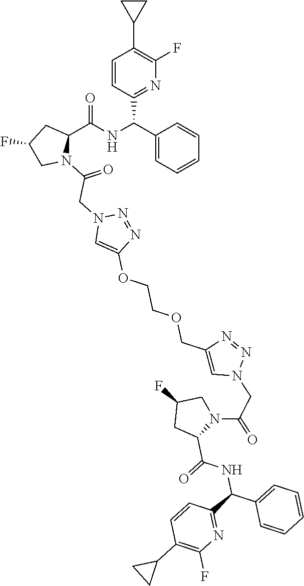 [SMILES: O=C(N[C@@H](c1ccccc1)c1ccc(C2CC2)c(F)n1)[C@@H]1C[C@@H](F)CN1C(=O)Cn1cc(COCCOc2cn(CC(=O)N3C[C@H](F)C[C@H]3C(=O)N[C@@H](c3ccccc3)c3ccc(C4CC4)c(F)n3)nn2)nn1] | (2S,4R)-N-((S)-(5-cyclopropyl-6- fluoropyridin-2-yl)(phenyl)methyl)- 1-(2-(4-(2-((1-(2-((2S,4R)-2-(((S)- (5-cyclopropyl-6-fluoropyridin-2- yl)(phenyl)methyl)carbamoyl)-4- fluoropyrrolidin-1-yl)-2-oxoethyl)- 1H-1,2,3-triazol-4- yl)methoxy)ethoxy)-1H-1,2,3- triazol-1-yl)acetyl)-4- fluoropyrrolidine-2-carboxamide |
| 9 | 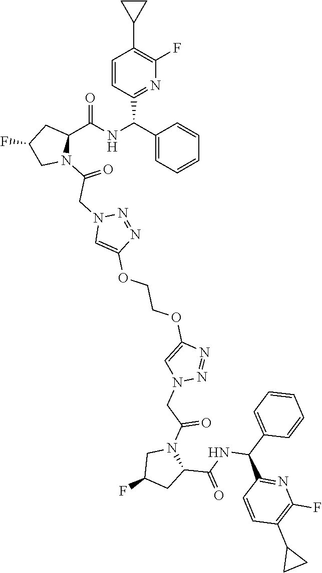 [SMILES: O=C(N[C@@H](c1ccccc1)c1ccc(C2CC2)c(F)n1)[C@@H]1C[C@@H](F)CN1C(=O)Cn1cc(OCCOc2cn(CC(=O)N3C[C@H](F)C[C@H]3C(=O)N[C@@H](c3ccccc3)c3ccc(C4CC4)c(F)n3)nn2)nn1] | (2S,2′S,4R,4′R)-1,1′-(2,2′-((ethane- 1,2-diylbis(oxy))bis(1H-1,2,3- triazole-4,1-diyl))bis(acetyl))bis(N- ((S)-(5-cyclopropyl-6- fluoropyridin-2-yl)(phenyl)methyl)- 4-fluoropyrrolidine-2-carboxamide) |
| 10 | 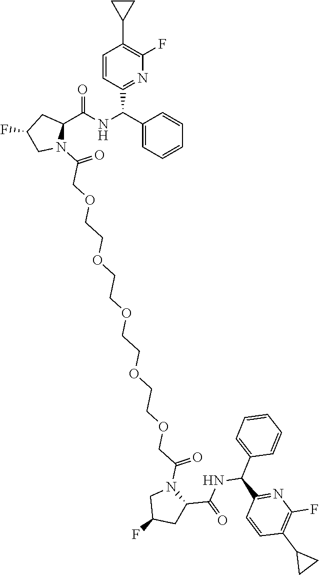 [SMILES: O=C(N[C@@H](c1ccccc1)c1ccc(C2CC2)c(F)n1)[C@@H]1C[C@@H](F)CN1C(=O)COCCOCCOCCOCCOCC(=O)N1C[C@H](F)C[C@H]1C(=O)N[C@@H](c1ccccc1)c1ccc(C2CC2)c(F)n1] | (2S,2′S,4R,4′R)-1,1′-(3,6,9,12,15- pentaoxaheptadecanedioyl)bis(N- ((S)-(5-cyclopropyl-6- fluoropyridin-2-yl)(phenyl)methyl)- 4-fluoropyrrolidine-2-carboxamide) |
| 11 | 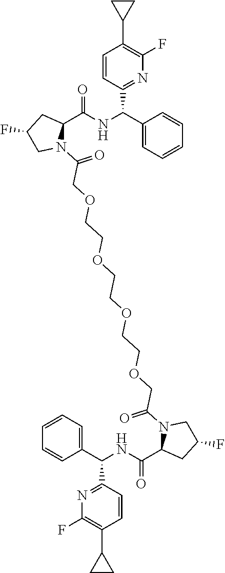 [SMILES: O=C(N[C@@H](c1ccccc1)c1ccc(C2CC2)c(F)n1)[C@@H]1C[C@@H](F)CN1C(=O)COCCOCCOCCOCC(=O)N1C[C@H](F)C[C@H]1C(=O)N[C@@H](c1ccccc1)c1ccc(C2CC2)c(F)n1] | (2S,2′S,4R,4′R)-1,1′-(3,6,9,12- tetraoxatetradecanedioyl)bis(N- ((S)-(5-cyclopropyl-6- fluoropyridin-2-yl)(phenyl)methyl)- 4-fluoropyrrolidine-2-carboxamide) |
| 12 | 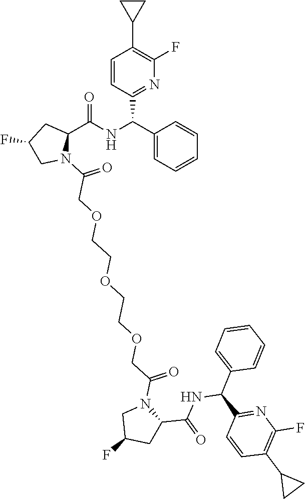 [SMILES: O=C(N[C@@H](c1ccccc1)c1ccc(C2CC2)c(F)n1)[C@@H]1C[C@@H](F)CN1C(=O)COCCOCCOCC(=O)N1C[C@H](F)C[C@H]1C(=O)N[C@@H](c1ccccc1)c1ccc(C2CC2)c(F)n1] | (2S,2′S,4R,4′R)-1,1′-(2,2′- ((oxybis(ethane-2,1- diyl))bis(oxy))bis(acetyl))bis(N- ((S)-(5-cyclopropyl-6- fluoropyridin-2-yl)(phenyl)methyl)- 4-fluoropyrrolidine-2-carboxamide) |
| 13 | 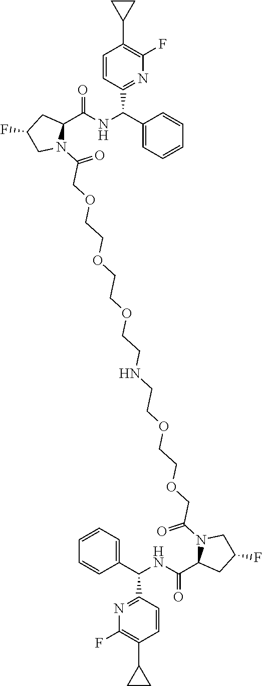 [SMILES: O=C(N[C@@H](c1ccccc1)c1ccc(C2CC2)c(F)n1)[C@@H]1C[C@@H](F)CN1C(=O)COCCOCCNCCOCCOCCOCC(=O)N1C[C@H](F)C[C@H]1C(=O)N[C@@H](c1ccccc1)c1ccc(C2CC2)c(F)n1] | (2S,2′S,4R,4′R)-1,1′-(3,6,12,15,18- pentaoxa-9-azaicosanedioyl)bis(N- ((S)-(5-cyclopropyl-6- fluoropyridin-2-yl)(phenyl)methyl)- 4-fluoropyrrolidine-2-carboxamide) |
| 14 | 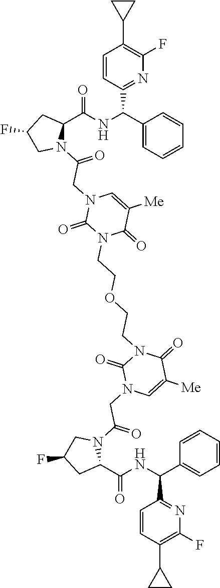 [SMILES: Cc1cn(CC(=O)N2C[C@H](F)C[C@H]2C(=O)N[C@@H](c2ccccc2)c2ccc(C3CC3)c(F)n2)c(=O)n(CCOCCn2c(=O)c(C)cn(CC(=O)N3C[C@H](F)C[C@H]3C(=O)N[C@@H](c3ccccc3)c3ccc(C4CC4)c(F)n3)c2=O)c1=O] | (2S,2′S,4R,4′R)-1,1′-(2,2′- ((oxybis(ethane-2,1-diyl))bis(5- methyl-2,4-dioxo-3,4- dihydropyrimidine-3,1(2H)- diyl))bis(acetyl))bis(N-((S)-(5- cyclopropyl-6-fluoropyridin-2- yl)(phenyl)methyl)-4- fluoropyrrolidine-2-carboxamide) |
| 15 | 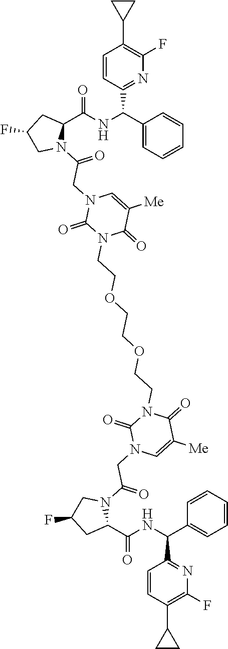 [SMILES: Cc1cn(CC(=O)N2C[C@H](F)C[C@H]2C(=O)N[C@@H](c2ccccc2)c2ccc(C3CC3)c(F)n2)c(=O)n(CCOCCOCCn2c(=O)c(C)cn(CC(=O)N3C[C@H](F)C[C@H]3C(=O)N[C@@H](c3ccccc3)c3ccc(C4CC4)c(F)n3)c2=O)c1=O] | (2S,2′S,4R,4′R)-1,1′-(2,2′-(((ethane- 1,2-diylbis(oxy))bis(ethane-2,1- diyl))bis(5-methyl-2,4-dioxo-3,4- dihydropyrimidine-3,1(2H)- diyl))bis(acetyl))bis(N-((S)-(5- cyclopropyl-6-fluoropyridin-2- yl)(phenyl)methyl)-4- fluoropyrrolidine-2-carboxamide) |
| 16 | 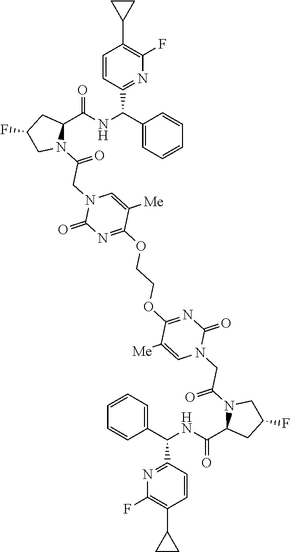 [SMILES: Cc1cn(CC(=O)N2C[C@H](F)C[C@H]2C(=O)N[C@@H](c2ccccc2)c2ccc(C3CC3)c(F)n2)c(=O)nc1OCCOc1nc(=O)n(CC(=O)N2C[C@H](F)C[C@H]2C(=O)N[C@@H](c2ccccc2)c2ccc(C3CC3)c(F)n2)cc1C] | (2S,2′S,4R,4′R)-1,1′-(2,2′-((ethane- 1,2-diylbis(oxy))bis(5-methyl-2- oxopyrimidine-4,1(2H)- diyl))bis(acetyl))bis(N-((S)-(5- cyclopropyl-6-fluoropyridin-2- yl)(phenyl)methyl)-4- fluoropyrrolidine-2-carboxamide) |
| 17 | | (2S,2′S,4R,4′R)-1,1′-(9-methyl- 3,6,12,15,18-pentaoxa-9- azaicosanedioyl)bis(N-((S)-(5- cyclopropyl-6-fluoropyridin-2- yl)(phenyl)methyl)-4- fluoropyrrolidine-2-carboxamide) |

In some embodiments, provided herein is a compound of formula (I), or any variation or embodiment thereof, or a stereoisomer or tautomer thereof, or a pharmaceutically acceptable salt of any of the foregoing, the compound, or a stereoisomer or tautomer thereof, or a pharmaceutically acceptable salt of any of the foregoing, is selected from the group consisting of:
1,1′-(2,2′-(((oxybis(ethane-2,1-diyl))bis(oxy))bis(1H-1,2,3-triazole-4,1-diyl))bis(acetyl))bis(N-((5-cyclopropyl-6-fluoropyridin-2-yl)(phenyl)methyl)-4-fluoropyrrolidine-2-carboxamide);
1,1′-(3,6,9,12,15,18,21-heptaoxatricosanedioyl)bis(N-((5-cyclopropyl-6-fluoropyridin-2-yl)(phenyl)methyl)-4-fluoropyrrolidine-2-carboxamide);
N¹,N³⁸-bis((5-cyclopropyl-6-fluoropyridin-2-yl)(phenyl)methyl)-3,6,9,12,15,18,21,24,27,30,33,36-dodecaoxaoctatriacontanediamide;
1,1′-(2,2′-(ethane-1,2-diylbis(oxy))bis(acetyl))bis(N-((5-cyclopropyl-6-fluoropyridin-2-yl)(phenyl)methyl)-4-fluoropyrrolidine-2-carboxamide);
1,1′-(3,6,9,12,15,18-hexaoxaicosanedioyl)bis(N-((5-cyclopropyl-6-fluoropyridin-2-yl)(phenyl)methyl)-4-fluoropyrrolidine-2-carboxamide);
N¹,N³⁵-bis((5-cyclopropyl-6-fluoropyridin-2-yl)(phenyl)methyl)-3,6,9,12,15,18,21,24,27,30,33-undecaoxapentatriacontanediamide;
1,1′-(2,2′-(((ethane-1,2-diylbis(oxy))bis(methylene))bis(1H-1,2,3-triazole-4,1-diyl))bis(acetyl))bis(N-((5-cyclopropyl-6-fluoropyridin-2-yl)(phenyl)methyl)-4-fluoropyrrolidine-2-carboxamide);
N-((5-cyclopropyl-6-fluoropyridin-2-yl)(phenyl)methyl)-1-(2-(4-(2-((1-(2-(2-(((5-cyclopropyl-6-fluoropyridin-2-yl)(phenyl)methyl)carbamoyl)-4-fluoropyrrolidin-1-yl)-2-oxoethyl)-1H-1,2,3-triazol-4-yl)methoxy)ethoxy)-1H-1,2,3-triazol-1-yl)acetyl)-4-fluoropyrrolidine-2-carboxamide;
1,1′-(2,2′-((ethane-1,2-diylbis(oxy))bis(1H-1,2,3-triazole-4,1-diyl))bis(acetyl))bis(N-((5-cyclopropyl-6-fluoropyridin-2-yl)(phenyl)methyl)-4-fluoropyrrolidine-2-carboxamide);
1,1′-(3,6,9,12,15-pentaoxaheptadecanedioyl)bis(N—((S)-(5-cyclopropyl-6-fluoropyridin-2-yl)(phenyl)methyl)-4-fluoropyrrolidine-2-carboxamide);
1,1′-(3,6,9,12-tetraoxatetradecanedioyl)bis(N-(5-cyclopropyl-6-fluoropyridin-2-yl)(phenyl)methyl)-4-fluoropyrrolidine-2-carboxamide);
1,1′-(2,2′-((oxybis(ethane-2,1-diyl))bis(oxy))bis(acetyl))bis(N-((5-cyclopropyl-6-fluoropyridin-2-yl)(phenyl)methyl)-4-fluoropyrrolidine-2-carboxamide);
N-[(5-cyclopropyl-6-fluoropyridin-2-yl)(phenyl)methyl]-1-[1-(2-{[(5-cyclopropyl-6-fluoropyridin-2-yl)(phenyl)methyl]carbamoyl}-4-fluoropyrrolidin-1-yl)-1-oxo-3,6,9,15,18-pentaoxa-12-azaicosanoyl]-4-fluoropyrrolidine-2-carboxamide; and
N-[(5-cyclopropyl-6-fluoropyridin-2-yl)(phenyl)methyl]-1-(2-{3-[2-(2-{3-[2-(2-{[(5-cyclopropyl-6-fluoropyridin-2-yl)(phenyl)methyl]carbamoyl}-4-fluoropyrrolidin-1-yl)-2-oxoethyl]-5-methyl-2,6-dioxo-1,2,3,6-tetrahydropyrimidin-1-yl}ethoxy)ethyl]-5-methyl-2,4-dioxo-1,2,3,4-tetrahydropyrimidin-1-yl}acetyl)-4-fluoropyrrolidine-2-carboxamide;
1,1′-(2,2′-(((ethane-1,2-diylbis(oxy))bis(ethane-2,1-diyl))bis(5-methyl-2,4-dioxo-3,4-dihydropyrimidine-3,1(2H)-diyl))bis(acetyl))bis(N-((5-cyclopropyl-6-fluoropyridin-2-yl)(phenyl)methyl)-4-fluoropyrrolidine-2-carboxamide);
1,1′-(2,2′-((ethane-1,2-diylbis(oxy))bis(5-methyl-2-oxopyrimidine-4,1(2H)-diyl))bis(acetyl))bis(N-((5-cyclopropyl-6-fluoropyridin-2-yl)(phenyl)methyl)-4-fluoropyrrolidine-2-carboxamide); and 1,1′-(9-methyl-3,6,12,15,18-pentaoxa-9-azaicosanedioyl)bis(N-((5-cyclopropyl-6-fluoropyridin-2-yl)(phenyl)methyl)-4-fluoropyrrolidine-2-carboxamide);
   or a pharmaceutically acceptable salt of any of the foregoing.

In some embodiments, provided herein is a compound of formula (I), or any variation or embodiment thereof, or a stereoisomer or tautomer thereof, or a pharmaceutically acceptable salt of any of the foregoing, the compound, or a stereoisomer or tautomer thereof, or a pharmaceutically acceptable salt of any of the foregoing, is selected from the group consisting of:
(2S,2′S,4R,4′R)-1,1′-(2,2′-(((oxybis(ethane-2,1-diyl))bis(oxy))bis(1H-1,2,3-triazole-4,1-diyl))bis(acetyl))bis(N—((S)-(5-cyclopropyl-6-fluoropyridin-2-yl)(phenyl)methyl)-4-fluoropyrrolidine-2-carboxamide);
(2S,2′S,4R,4′R)-1,1′-(3,6,9,12,15,18,21-heptaoxatricosanedioyl)bis(N—((S)-(5-cyclopropyl-6-fluoropyridin-2-yl)(phenyl)methyl)-4-fluoropyrrolidine-2-carboxamide);
N¹,N³⁸-bis((S)-(5-cyclopropyl-6-fluoropyridin-2-yl)(phenyl)methyl)-3,6,9,12,15,18,21,24,27,30,33,36-dodecaoxaoctatriacontanediamide;
(2S,2′S,4R,4′R)-1,1′-(2,2′-(ethane-1,2-diylbis(oxy))bis(acetyl))bis(N—((S)-(5-cyclopropyl-6-fluoropyridin-2-yl)(phenyl)methyl)-4-fluoropyrrolidine-2-carboxamide);
(2S,2′S,4R,4′R)-1,1′-(3,6,9,12,15,18-hexaoxaicosanedioyl)bis(N—((S)-(5-cyclopropyl-6-fluoropyridin-2-yl)(phenyl)methyl)-4-fluoropyrrolidine-2-carboxamide);
N¹,N³⁵-bis((S)-(5-cyclopropyl-6-fluoropyridin-2-yl)(phenyl)methyl)-3,6,9,12,15,18,21,24,27,30,33-undecaoxapentatriacontanediamide;
(2S,2′S,4R,4′R)-1,1′-(2,2′-(((ethane-1,2-diylbis(oxy))bis(methylene))bis(1H-1,2,3-triazole-4,1-diyl))bis(acetyl))bis(N—((S)-(5-cyclopropyl-6-fluoropyridin-2-yl)(phenyl)methyl)-4-fluoropyrrolidine-2-carboxamide);
(2S,4R)—N—((S)-(5-cyclopropyl-6-fluoropyridin-2-yl)(phenyl)methyl)-1-(2-(4-(2-((1-(2-((2S,4R)-2-(((S)-(5-cyclopropyl-6-fluoropyridin-2-yl)(phenyl)methyl)carbamoyl)-4-fluoropyrrolidin-1-yl)-2-oxoethyl)-1H-1,2,3-triazol-4-yl)methoxy)ethoxy)-1H-1,2,3-triazol-1-yl)acetyl)-4-fluoropyrrolidine-2-carboxamide;
(2S,2′S,4R,4′R)-1,1′-(2,2′-((ethane-1,2-diylbis(oxy))bis(1H-1,2,3-triazole-4,1-diyl))bis(acetyl))bis(N—((S)-(5-cyclopropyl-6-fluoropyridin-2-yl)(phenyl)methyl)-4-fluoropyrrolidine-2-carboxamide);
(2S,2′S,4R,4′R)-1,1′-(3,6,9,12,15-pentaoxaheptadecanedioyl)bis(N—((S)-(5-cyclopropyl-6-fluoropyridin-2-yl)(phenyl)methyl)-4-fluoropyrrolidine-2-carboxamide);
(2S,2′S,4R,4′R)-1,1′-(3,6,9,12-tetraoxatetradecanedioyl)bis(N—((S)-(5-cyclopropyl-6-fluoropyridin-2-yl)(phenyl)methyl)-4-fluoropyrrolidine-2-carboxamide);
(2S,2′S,4R,4′R)-1,1′-(2,2′-((oxybis(ethane-2,1-diyl))bis(oxy))bis(acetyl))bis(N—((S)-(5-cyclopropyl-6-fluoropyridin-2-yl)(phenyl)methyl)-4-fluoropyrrolidine-2-carboxamide);
(2S,2′S,4R,4′R)-1,1′-(3,6,12,15,18-pentaoxa-9-azaicosanedioyl)bis(N—((S)-(5-cyclopropyl-6-fluoropyridin-2-yl)(phenyl)methyl)-4-fluoropyrrolidine-2-carboxamide); and
(2S,2′S,4R,4′R)-1,1′-(2,2′-((oxybis(ethane-2,1-diyl))bis(5-methyl-2,4-dioxo-3,4-dihydropyrimidine-3,1(2H)-diyl))bis(acetyl))bis(N—((S)-(5-cyclopropyl-6-fluoropyridin-2-yl)(phenyl)methyl)-4-fluoropyrrolidine-2-carboxamide);
(2S,2′S,4R,4′R)-1,1′-(2,2′-(((ethane-1,2-diylbis(oxy))bis(ethane-2,1-diyl))bis(5-methyl-2,4-dioxo-3,4-dihydropyrimidine-3,1(2H)-diyl))bis(acetyl))bis(N—((S)-(5-cyclopropyl-6-fluoropyridin-2-yl)(phenyl)methyl)-4-fluoropyrrolidine-2-carboxamide);
(2S,2′S,4R,4′R)-1,1′-(2,2′-((ethane-1,2-diylbis(oxy))bis(5-methyl-2-oxopyrimidine-4,1(2H)-diyl))bis(acetyl))bis(N—((S)-(5-cyclopropyl-6-fluoropyridin-2-yl)(phenyl)methyl)-4-fluoropyrrolidine-2-carboxamide); and
(2S,2′S,4R,4′R)-1,1′-(9-methyl-3,6,12,15,18-pentaoxa-9-azaicosanedioyl)bis(N—((S)-(5-cyclopropyl-6-fluoropyridin-2-yl)(phenyl)methyl)-4-fluoropyrrolidine-2-carboxamide);
   or a stereoisomer or tautomer thereof, or a pharmaceutically acceptable salt of any of the foregoing.

### Methods of Treatment

Provided herein is a method for simultaneously engaging two GYS1 monomers within a single tetrameric complex. This disclosure demonstrates that linking two GYS1 inhibitors together with a variety of linkers creates a bivalent inhibitor with the potential to simultaneously engage two GYS1 monomers within a single tetrameric complex.

Provided herein is a method of modulating GYS1 in a cell, comprising exposing the cell to (i) a composition comprising an effective amount of a compound of formula (I), or any variation or embodiment thereof, or a stereoisomer or tautomer thereof, or a pharmaceutically acceptable salt of any of the foregoing, or (ii) a pharmaceutical composition, comprising an effective amount of a compound of formula (I), or any variation or embodiment thereof, or a stereoisomer or tautomer thereof, or a pharmaceutically acceptable salt of any of the foregoing, and one or more pharmaceutically acceptable excipients.

Provided herein is a method of inhibiting GYS1 in a cell, comprising exposing the cell to (i) a composition comprising an effective amount of a compound of formula (I), or (ii) a pharmaceutical composition, comprising an effective amount of a compound of formula (I), or any variation or embodiment thereof, or a stereoisomer or tautomer thereof, or a pharmaceutically acceptable salt of any of the foregoing, and one or more pharmaceutically acceptable excipients. In some embodiments, the compound or formula (I) is selective for GYS1 over GYS2. In some embodiments, the compound or formula (I) is greater than 500 or 1,000 or 1,500 or 1,700-fold selective for GYS1 over GYS2.

Provided herein is a method of inhibiting GYS1 in a cell, comprising exposing the cell to (i) an effective amount of a compound of formula (I), or any variation or embodiment thereof, or a stereoisomer or tautomer thereof, or a pharmaceutically acceptable salt of any of the foregoing, or (ii) a pharmaceutical composition, comprising an effective amount of a compound of formula (I), or any variation or embodiment thereof, or a stereoisomer or tautomer thereof, or a pharmaceutically acceptable salt of any of the foregoing, and one or more pharmaceutically acceptable excipients.

Provided herein is a method of reducing tissue glycogen stores in an individual in need thereof, comprising administering to the individual an effective amount of (i) an effective amount of a compound of formula (I), or any variation or embodiment thereof, or a stereoisomer or tautomer thereof, or a pharmaceutically acceptable salt of any of the foregoing, or (ii) a pharmaceutical composition, comprising an effective amount of a compound of formula (I), or any variation or embodiment thereof, or a stereoisomer or tautomer thereof, or a pharmaceutically acceptable salt of any of the foregoing, and one or more pharmaceutically acceptable excipients. In some embodiments, the compound of formula (I) is selective for GYS1 over GYS2. In some embodiments, the compound of formula (I) is greater than 500 or 1,000 or 1,500 or 1,700-fold selective for GYS1 over GYS2. In some embodiments, the individual has a GYS1-mediated disease, disorder, or condition is selected from the group consisting of Pompe disease, Cori disease (GSD III), adult polyglucosan body disease (APBD), and Lafora disease. In some embodiments, the GYS1-mediated disease, disorder, or condition is cancer. In some embodiments, the GYS1-mediated disease, disorder, or condition is selected from the group consisting of Ewing sarcoma (ES), clear cell renal cell carcinoma (ccRCC), glycogen rich clear cell carcinoma (GRCC) breast cancer, non-small-cell lung carcinoma (NSCLC), and acute myeloid leukemia (AML). In some embodiments, the GYS1-mediated disease, disorder, or condition is Pompe disease. In some embodiments, the GYS1-mediated disease, disorder, or condition is late-onset Pompe disease (LOPD).

Provided herein is a method of reducing tissue glycogen stores in an individual in need thereof, comprising administering to the individual (i) a composition comprising an effective amount of a compound of formula (I), or any variation or embodiment thereof, or a stereoisomer or tautomer thereof, or a pharmaceutically acceptable salt of any of the foregoing, or (ii) a pharmaceutical composition, comprising an effective amount of a compound of formula (I), or any variation or embodiment thereof, or a stereoisomer or tautomer thereof, or a pharmaceutically acceptable salt of any of the foregoing, and one or more pharmaceutically acceptable excipients.

Provided herein is a method of inhibiting glycogen synthesis in an individual in need thereof, comprising administering to the individual an effective amount of (i) an effective amount of a compound of formula (I), or any variation or embodiment thereof, or a stereoisomer or tautomer thereof, or a pharmaceutically acceptable salt of any of the foregoing, or (ii) a pharmaceutical composition, comprising an effective amount of a compound of formula (I), or any variation or embodiment thereof, or a stereoisomer or tautomer thereof, or a pharmaceutically acceptable salt of any of the foregoing, and one or more pharmaceutically acceptable excipients. In some embodiments, the pharmaceutical composition is (i) a composition comprising an effective amount of a compound of formula (I), or any variation or embodiment thereof, or a stereoisomer or tautomer thereof, or a pharmaceutically acceptable salt of any of the foregoing, or (ii) a pharmaceutical composition, comprising an effective amount of a compound of formula (I), or any variation or embodiment thereof, or a stereoisomer or tautomer thereof, or a pharmaceutically acceptable salt of any of the foregoing, and one or more pharmaceutically acceptable excipients. In some embodiments, the compounds and/or compositions inhibit the hGYS enzyme, and subsequently, the glycogen synthesis in cells.

Provided herein is a method of treating a GYS1-mediated disease, disorder, or condition in an individual in need thereof, comprising subjecting the individual to glycogen substrate reduction therapy comprising administering to the individual an effective amount of (i) an effective amount of a compound of formula (I), or any variation or embodiment thereof, or a stereoisomer or tautomer thereof, or a pharmaceutically acceptable salt of any of the foregoing, or (ii) a pharmaceutical composition, comprising an effective amount of a compound of formula (I), or any variation or embodiment thereof, or a stereoisomer or tautomer thereof, or a pharmaceutically acceptable salt of any of the foregoing, and one or more pharmaceutically acceptable excipients. In some embodiments, the compound of formula (I) is selective for GYS1 over GYS2. In some embodiments, the compound of formula (I) is greater than 500 or 1,000 or 1,500 or 1,700-fold selective for GYS1 over GYS2. In some embodiments, the GYS1-mediated disease, disorder, or condition is selected from the group consisting of Pompe disease, Cori disease (GSD III), adult polyglucosan body disease (APBD), and Lafora disease. In some embodiments, the GYS1-mediated disease, disorder, or condition is cancer. In some embodiments, the GYS1-mediated disease, disorder, or condition is selected from the group consisting of Ewing sarcoma (ES), clear cell renal cell carcinoma (ccRCC), glycogen rich clear cell carcinoma (GRCC) breast cancer, non-small-cell lung carcinoma (NSCLC), and acute myeloid leukemia (AML). In some embodiments, the GYS1-mediated disease, disorder, or condition is Pompe disease.

Provided herein is a method of treating a GYS1-mediated disease, disorder, or condition in an individual in need thereof, comprising administering to the individual (i) a composition comprising an effective amount of a compound of formula (I), or any variation or embodiment thereof, or a stereoisomer or tautomer thereof, or a pharmaceutically acceptable salt of any of the foregoing, or (ii) a pharmaceutical composition, comprising an effective amount of a compound of formula (I), or any variation or embodiment thereof, or a stereoisomer or tautomer thereof, or a pharmaceutically acceptable salt of any of the foregoing, and one or more pharmaceutically acceptable excipients. In some embodiments, the GYS1-mediated disease, disorder, or condition is selected from the group consisting of Pompe disease, Cori disease (GSD III), adult polyglucosan body disease (APBD), and Lafora disease. In some embodiments, the GYS1-mediated disease, disorder, or condition is cancer. In some embodiments, the GYS1-mediated disease, disorder, or condition is selected from the group consisting of Ewing sarcoma (ES), clear cell renal cell carcinoma (ccRCC), glycogen rich clear cell carcinoma (GRCC) breast cancer, non-small-cell lung carcinoma (NSCLC), and acute myeloid leukemia (AML).

Provided herein is a method of treating a GYS1-mediated disease, disorder, or condition in an individual in need thereof, comprising administering to the individual (i) a composition comprising a compound of formula (I), or any variation or embodiment thereof, or a stereoisomer or tautomer thereof, or a pharmaceutically acceptable salt of any of the foregoing, or (ii) a pharmaceutical composition, comprising a compound of formula (I), or any variation or embodiment thereof, or a stereoisomer or tautomer thereof, or a pharmaceutically acceptable salt of any of the foregoing, and one or more pharmaceutically acceptable excipients. In some embodiments, the GYS1-mediated disease, disorder, or condition is selected from the group consisting of Pompe disease, Cori disease (GSD III), adult polyglucosan body disease (APBD), and Lafora disease. In some embodiments, the GYS1-mediated disease, disorder, or condition is cancer. In some embodiments, the GYS1-mediated disease, disorder, or condition is selected from the group consisting of Ewing sarcoma (ES), clear cell renal cell carcinoma (ccRCC), glycogen rich clear cell carcinoma (GRCC) breast cancer, non-small-cell lung carcinoma (NSCLC), and acute myeloid leukemia (AML).

Provided herein is a method of treating a glycogen storage disease, disorder, or condition in an individual in need thereof, comprising subjecting the individual to glycogen substrate reduction therapy comprising administering to the individual an effective amount of (i) a compound of formula (I), or any variation or embodiment thereof, or a stereoisomer or tautomer thereof, or a pharmaceutically acceptable salt of any of the foregoing, or (ii) a pharmaceutical composition, comprising an effective amount of a compound of formula (I), or any variation or embodiment thereof, or a stereoisomer or tautomer thereof, or a pharmaceutically acceptable salt of any of the foregoing, and one or more pharmaceutically acceptable excipients. In some embodiments, the compound of formula (I) is selective for GYS1 over GYS2. In some embodiments, the compound of formula (I) is greater than 500 or 1,000 or 1,500 or 1,700-fold selective for GYS1 over GYS2. In some embodiments, the level of glycogen in the individual is reduced upon treatment. In some embodiments, the level of glycogen in muscle is reduced. In some embodiments, the level of glycogen is skeletal muscle is reduced. In some embodiments, the level of glycogen is reduced at least 10%, at least 20%, at least 30% or at least 50% upon administration of the compound. In some embodiments, the compounds provided herein are effective for treating a lysosomal disorder. In some embodiments, the glycogen storage disease, disorder, or condition is selected from the group consisting of Pompe disease, Cori disease (GSD III), adult polyglucosan body disease (APBD), and Lafora disease. In some embodiments, the glycogen storage disease, disorder, or condition is Pompe disease. In some embodiments, the individual has late onset Pompe Disease.

Provided herein is a method of treating a glycogen storage disease, disorder, or condition in an individual in need thereof, comprising subjecting the individual to glycogen substrate reduction therapy comprising administering to the individual (i) a compound of formula (I), or any variation or embodiment thereof, or a stereoisomer or tautomer thereof, or a pharmaceutically acceptable salt of any of the foregoing, or (ii) a pharmaceutical composition, comprising a compound of formula (I), or any variation or embodiment thereof, or a stereoisomer or tautomer thereof, or a pharmaceutically acceptable salt of any of the foregoing, and one or more pharmaceutically acceptable excipients. In some embodiments, the compound of formula (I) is selective for GYS1 over GYS2. In some embodiments, the compound of formula (I) is greater than 500 or 1,000 or 1,500 or 1,700-fold selective for GYS1 over GYS2. In some embodiments, the level of glycogen in the individual is reduced upon treatment. In some embodiments, the level of glycogen in muscle is reduced. In some embodiments, the level of glycogen is skeletal muscle is reduced. In some embodiments, the level of glycogen is reduced at least 10%, at least 20%, at least 30% or at least 50% upon administration of the compound. In some embodiments, the compounds provided herein are effective for treating a lysosomal disorder. In some embodiments, the glycogen storage disease, disorder, or condition is selected from the group consisting of Pompe disease, Cori disease (GSD III), adult polyglucosan body disease (APBD), and Lafora disease. In some embodiments, the glycogen storage disease, disorder, or condition is Pompe disease. In some embodiments, the individual has late onset Pompe Disease.

Provided herein is a method of treating a glycogen storage disease, disorder, or condition in an individual in need thereof, comprising administering to the individual (i) a composition comprising an effective amount of a compound of formula (I), or any variation or embodiment thereof, or a stereoisomer or tautomer thereof, or a pharmaceutically acceptable salt of any of the foregoing, or (ii) a pharmaceutical composition, comprising an effective amount of a compound of formula (I), or any variation or embodiment thereof, or a stereoisomer or tautomer thereof, or a pharmaceutically acceptable salt of any of the foregoing, and one or more pharmaceutically acceptable excipients. In some embodiments, the level of glycogen in the individual is reduced upon treatment. In some embodiments, the level of glycogen in muscle is reduced. In some embodiments, the level of glycogen is skeletal muscle is reduced. In some embodiments, the level of glycogen is reduced at least 10%, at least 20%, at least 30% or at least 50% upon administration of the compound. In some embodiments, the compounds provided herein are effective for treating a lysosomal disorder. In some embodiments, the glycogen storage disease, disorder, or condition is selected from the group consisting of Pompe disease, Cori disease (GSD III), adult polyglucosan body disease (APBD), and Lafora disease.

Provided herein is a method of treating a glycogen storage disease, disorder, or condition in an individual in need thereof, comprising administering to the individual (i) a composition comprising a compound of formula (I), or any variation or embodiment thereof, or a stereoisomer or tautomer thereof, or a pharmaceutically acceptable salt of any of the foregoing, or (ii) a pharmaceutical composition, comprising a compound of formula (I), or any variation or embodiment thereof, or a stereoisomer or tautomer thereof, or a pharmaceutically acceptable salt of any of the foregoing, and one or more pharmaceutically acceptable excipients. In some embodiments, the level of glycogen in the individual is reduced upon treatment. In some embodiments, the level of glycogen in muscle is reduced. In some embodiments, the level of glycogen is skeletal muscle is reduced. In some embodiments, the level of glycogen is reduced at least 10%, at least 20%, at least 30% or at least 50% upon administration of the compound. In some embodiments, the compounds provided herein are effective for treating a lysosomal disorder. In some embodiments, the glycogen storage disease, disorder, or condition is selected from the group consisting of Pompe disease, Cori disease (GSD III), adult polyglucosan body disease (APBD), and Lafora disease.

Provided herein is a method of treating Pompe disease in an individual in need thereof, comprising administering to the individual (i) a composition comprising an effective amount of a compound of formula (I), or any variation or embodiment thereof, or a stereoisomer or tautomer thereof, or a pharmaceutically acceptable salt of any of the foregoing, or (ii) a pharmaceutical composition, comprising an effective amount of a compound of formula (I), or any variation or embodiment thereof, or a stereoisomer or tautomer thereof, or a pharmaceutically acceptable salt of any of the foregoing, and one or more pharmaceutically acceptable excipients. In some embodiments, the individual has infant onset Pompe disease. In some embodiments, the individual has non-classic infant-onset Pompe disease. In some embodiments, the individual has late-onset Pompe disease. In some embodiments, the individual has a deficiency in acid alfa glucosidase (GAA). In some embodiments, the individual has reduced expression of GAA.

Provided herein is a method of treating Pompe disease in an individual in need thereof, comprising administering to the individual (i) a composition comprising a compound of formula (I), or any variation or embodiment thereof, or a stereoisomer or tautomer thereof, or a pharmaceutically acceptable salt of any of the foregoing, or (ii) a pharmaceutical composition, comprising a compound of formula (I), or any variation or embodiment thereof, or a stereoisomer or tautomer thereof, or a pharmaceutically acceptable salt of any of the foregoing, and one or more pharmaceutically acceptable excipients. In some embodiments, the individual has infant onset Pompe disease. In some embodiments, the individual has non-classic infant-onset Pompe disease. In some embodiments, the individual has late-onset Pompe disease. In some embodiments, the individual has a deficiency in acid alfa glucosidase (GAA). In some embodiments, the individual has reduced expression of GAA.

In some embodiments, the methods provided herein reduce and/or eliminate one or more symptoms associated with Pompe disease. In some embodiments, the methods reduce and/or eliminate weak muscles, poor muscle tone, enlarged liver, failure to grow and gain weight, trouble breathing, feeding problems, infections in the respiratory system, problems with hearing, motor skill delay, heart enlargement, tiredness, lung infection, frequent falling, or irregular heartbeat. In some embodiments, the methods herein delay progression of Pompe disease.

In some embodiments, the methods provided herein increase the lifespan of the individual.

In some embodiments, the lifespan is increased at least 5, at least 10, or at least 20 years upon treatment.

In some embodiments, the methods provided herein prevent, reduce, or delay muscle weakness. In some embodiments, muscle weakness is determined by manual muscle testing, sit to stand test, heel-raise test, hand-held dynamometry, or hand grip dynamometry. In some embodiments, strength is graded according to the following scale: 0: No visible muscle contraction; 1: Visible muscle contraction with no or trace movement; 2: Limb movement, but not against gravity; 3: Movement against gravity but not resistance; 4: Movement against at least some resistance supplied by the examiner; 5: Full strength.

Also provided herein is a method of inhibiting a GYS1 enzyme in an individual comprising administering an effective amount of a compound of formula (I) or a pharmaceutically acceptable salt thereof to the individual. In some embodiments the GYS1 enzyme is human GYS1 (hGYS1). In some embodiments, the compounds provided herein inhibit GYS1 at a concentration of less than 10 μM, less than 1 μM, less than 0.5 μM, or less than 0.1 μM. In some embodiments, the compounds provided herein inhibit GYS1 at a concentration of 1-10 μM, 0.01 to 1 μM, or 0.01 to 10 μM.

In some embodiments, the compounds have an IC₅₀ of less than 10 nM, less than 10 μM, less than 1 μM, less than 0.5 μM, or less than 0.1 μM. In some embodiments, the compounds provided herein have an IC₅₀ of 1 to 10 nM, 1 to 10 μM, 0.01 to 1 μM, 0.01 to 10 μM, or 0.001 to 0.01 μM.

In some embodiments, glycogen synthesis is inhibited upon administration of a compound provided herein. In some embodiments, glycogen synthesis is reduced at least 10%, at least 20%, at least 40% or at least 50% upon administration.

In some embodiments, the individual receiving treatment is a juvenile human or an infant. In some embodiments, the individual is less than 10 years old, less than 9 years old, less than 8 years old, less than 7 years old, less than 6 years old, less than 5 years old, less than 4 years old, less than 3 years old, less than 2 years old, or less than one year old.

In some embodiments, the methods further comprise enzyme replacement therapy (ERT). Exemplary ERTs include alglucosidase alfa (human recombinant alpha-glucosidase (human GAA)) and those described in Byrne B J et al (2011). Pompe disease: design, methodology, and early findings from the Pompe Registry. Mol Genet Metab 103: 1-11 (herein incorporated by reference in its entirety). In some embodiments, the ERT is selected from the group consisting of Myozyme and Lumizyme. In some embodiments, the ERT is Myozyme. In some embodiments, the ERT is Lumizyme. In some embodiments, the individual has an advanced glycogen storage disease. In some embodiments, the individual has late onset Pompe Disease. Thus, provided herein is a method of treating a GYS1-mediated disease, disorder, or condition in an individual in need thereof, comprising subjecting the individual to (a) glycogen substrate reduction therapy, such as administering to the individual (i) an effective amount of a compound of formula (I), or any variation or embodiment thereof, or a stereoisomer or tautomer thereof, or a pharmaceutically acceptable salt of any of the foregoing, or (ii) a pharmaceutical composition, comprising an effective amount of a compound of formula (I), and one or more pharmaceutically acceptable excipients and (b) enzyme replacement therapy. In some embodiments, the GYS1-mediated disease, disorder, or condition is Pompe disease, such as late-onset Pompe disease. In some embodiments, the compound of formula (I) is selective for GYS1 over GYS2. In some embodiments, the compound of formula (I) is greater than 500 or 1,000 or 1,500 or 1,700-fold selective for GYS1 over GYS2.

In some embodiments, the individual has a mutation in the GAA gene. In some embodiments, the mutation reduces the level of GAA protein. In some embodiments, the mutation is a loss-of-function mutation. In some embodiments, the mutation is a missense mutation. In some embodiments, the mutation is a deletion. In some embodiments, the mutation is a recessive mutation. In some embodiments, the mutation is a splicing variant.

In some embodiments of the foregoing, the administration is oral administration.

### Kits

The present disclosure further provides kits for carrying out the methods of the invention. The kits may comprise a compound or pharmaceutically acceptable salt thereof as described herein and suitable packaging. The kits may comprise one or more containers comprising any compound described herein. In one aspect, a kit includes a compound of the disclosure or a pharmaceutically acceptable salt thereof, and a label and/or instructions for use of the compound in the treatment of a disease or disorder described herein. The kits may comprise a unit dosage form of the compound.

Provided herein are kits, comprising (i) a composition comprising an effective amount of a compound of formula (I), or any variation or embodiment thereof, or a stereoisomer or tautomer thereof, or a pharmaceutically acceptable salt of any of the foregoing, and (ii) instructions for use in treating an GYS1-mediated disease, disorder, or condition in an individual in need thereof. Also provided herein are kits, comprising (i) a pharmaceutical composition comprising an effective amount of a compound of formula (I), or any variation or embodiment thereof, or a stereoisomer or tautomer thereof, or a pharmaceutically acceptable salt of any of the foregoing, and one or more pharmaceutically acceptable excipients; and (ii) instructions for use in treating an GYS1-mediated disease, disorder, or condition in an individual in need thereof

Articles of manufacture are also provided, wherein the article of manufacture comprises a compound of formula (I), or any variation or embodiment thereof, as described elsewhere herein, or a stereoisomer or tautomer thereof, or a pharmaceutically acceptable salt of any of the foregoing, in a suitable container. Also provided herein are articles of manufacture, comprising a pharmaceutical composition comprising a compound of formula (I), or any variation or embodiment thereof, as described elsewhere herein, or a stereoisomer or tautomer thereof, or a pharmaceutically acceptable salt of any of the foregoing, in a suitable container. The container may be a vial, jar, ampoule, preloaded syringe, or intravenous bag.

### Methods of Preparing

The present disclosure further provides processes for preparing the compounds of present invention. In some aspect, provided herein are processes of preparing a compound of formula (I), or any embodiment or variation thereof, such as a compound of formula (II), (II-A), (III), (II-A), (IV), (IV-A), (V), or (V-A), or a stereoisomer or tautomer thereof, or a pharmaceutically acceptable salt of any of the foregoing.

In some embodiments, a process for preparing a compound of formula (I), or a stereoisomer or tautomer thereof, or a pharmaceutically acceptable salt of any of the foregoing, comprises reacting a compound of formula (I-1B):

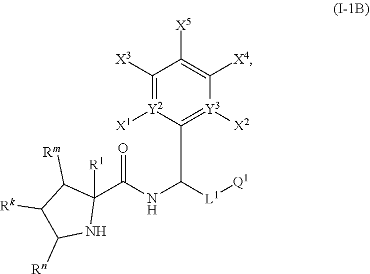

Y² and Y³ are each C, or
one of Y² and Y³ is N and the other of Y² and Y³ is C;
X¹ and X² are each independently H, C₁₋₆alkyl, or C₁₋₆alkoxy;
X³ and X⁴ are each independently H, halo, C₁₋₆alkyl, C₁₋₆alkoxy, or 5-20 membered heteroaryl, wherein the C₁₋₆alkyl of X³ and X⁴ is optionally substituted with one of more halo;
X⁵ is H, C₁₋₆alkyl, C₁₋₆alkoxy, or C₃₋₁₀cycloalkyl;
either
(1) L¹ is absent; and
Q¹ is selected from (i) to (iv):
(i) phenyl, wherein the phenyl of Q¹ is substituted with one or more halo, C₁₋₆alkyl, C₂₋₆alkenyl, —NH₂, —NH—C(O)—(C₁₋₆alkyl), —NH—C(O)-(3-15 membered heterocyclyl), C₃₋₁₀cycloalkyl, or 5-20 membered heteroaryl, wherein
   the C₁₋₆alkyl is optionally substituted with one or more halo, —NH—C(O)—NH(C₁₋₆alkyl), —NH—C(O)—C₁₋₆alkyl, or —NH—C(O)—C₁₋₆alkoxy,
   the C₃₋₁₀cycloalkyl is optionally substituted with one or more halo or C₁₋₆alkyl, and
   the 5-20 membered heteroaryl is optionally substituted with one or more C₁₋₆alkyl,
(ii) 3-15 membered heterocyclyl, wherein the 3-15 membered heterocyclyl of Q¹ is optionally substituted with one or more oxo, or C₁₋₆alkyl,
(iii) 5-20 membered heteroaryl, wherein the 5-20 membered heteroaryl of Q¹ is optionally substituted with one or more halo, C₁₋₆alkyl, C₂₋₆alkenyl, C₁₋₆alkoxy, —NH₂, or C₃₋₁₀cycloalkyl, wherein,
   the C₁₋₆alkyl is optionally substituted with one or more halo, and
   the C₃₋₁₀cycloalkyl is optionally substituted with one or more halo or C₁₋₆alkyl, and
(iv) C₃₋₁₀cycloalkyl;
or
(2) L¹ is —CH₂—; and
Q¹ is C₃₋₁₀cycloalkyl;
R¹ is H or C₁₋₆alkyl;
R^{k} is H, halo, —OH, —NH₂, or —NH—C(O)C₁₋₆alkyl;
R^{m} is H, —OH, or C₁₋₆alkyl;
Rⁿ is H, C₁₋₆alkyl, or C₃₋₁₀cycloalkyl or Rⁿ taken together with the carbon atom to which it is attached forms C₃₋₅ cycloalkyl; and
or R^{k} is taken together with either R^{m} or Rⁿ, and the atoms to which they are attached, to form cyclopropyl;
   with a compound of formula (L-1):

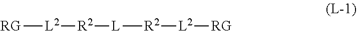

wherein
L² is —C(O)— or —S(O)₂—;
R² is selected from (i) to (vii):
(i) C₁₋₆alkyl, wherein the C₁₋₆alkyl of R² is optionally substituted with one or more R^{a}, wherein R^{a} is:
   (a) —OH,
   (b) cyano,
   (c) C₂₋₆alkynyl,
   (d) C₆₋₂₀aryl, wherein the C₆₋₂₀aryl of R^{a} is optionally substituted with one or more halo, cyano, C₁₋₆alkoxy, or —NH—C(O)—C₁₋₆alkyl,
   (e) 3-15 membered heterocyclyl, wherein the 3-15 membered heterocyclyl of R^{a} is optionally substituted with one or more R^{c}, wherein
      R^{c} is halo, oxo, C₁₋₆alkyl, C₁₋₆alkoxy, —C(O)—C₁₋₆alkyl, or —C(O)—C₁₋₆alkoxy, wherein
         the C₁₋₆alkyl of R^{c} is optionally substituted with one or more halo or C₂₋₆alkynyl, and
         the —C(O)—C₁₋₆alkoxy of R^{c} is optionally substituted with one or more halo,
   (f) —N(R^{c})(R^{d}), wherein R^{c} and R^{d} of —N(R^{c})(R^{d}) are, independently of each other, H, C₁₋₆alkyl, —C(O)—C₁₋₆alkyl, —C(O)—C₁₋₆alkoxy, —C(O)—NH₂, —C(O)—NH(C₁₋₆alkyl), —C(O)—N(C₁₋₆alkyl)₂, —C(O)-(3-15 membered heterocyclyl), —CH₂—C(O)—NH₂, 3-15 membered heterocyclyl, or 5-20 membered heteroaryl, wherein
      the C₁₋₆alkyl of R^{c} or R^{d} is optionally substituted with one or more —C(O)—NH₂,
      the —C(O)—C₁₋₆alkyl of R^{c} or R^{d} is optionally substituted with one or more halo,
      the 3-15 membered heterocyclyl and the 5-20 membered heteroaryl of R^{c} or R^{d} are independently optionally substituted with one or more C₁₋₆alkyl,
      the —C(O)-(3-15 membered heterocyclyl) of R^{c} or R^{d} is optionally substituted with one or more halo, —C(O)—C₁₋₆alkoxy, or C₁₋₆alkyl, wherein the C₁₋₆alkyl is optionally substituted with one or more halo, C₁₋₆alkoxy, or C₃₋₁₀cycloalkyl, and
      the C₁₋₆alkyl of the —C(O)—N(C₁₋₆alkyl)₂ of R^{c} or R^{d} are, independently of each other, optionally substituted with one or more halo or C₆₋₂₀aryl,
   (g) —O—R^{e}, wherein R^{e} is C₁₋₆alkyl, C₆₋₂₀aryl, —C(O)-(3-15 membered heterocyclyl), —C(O)—N—(C₁₋₆alkyl)₂, or 5-20 membered heteroaryl, wherein
      the C₁₋₆alkyl of R^{e} is optionally substituted with one or more C₁₋₆alkoxy, wherein the C₁₋₆alkoxy is optionally substituted with one or more C₂₋₆alkynyl,
      the C₆₋₂₀aryl of R^{e} is optionally substituted with one or more C₁₋₆alkyl, and
      the —C(O)-(3-15 membered heterocyclyl) of R^{e} is optionally substituted with one or more C₁₋₆alkyl, C₁₋₆alkoxy, or —C(O)—C₁₋₆alkoxy, wherein the C₁₋₆alkyl is optionally substituted with one or more halo, C₁₋₆alkoxy, or C₃₋₁₀cycloalkyl,
   (h) —C(O)—R^{e}, wherein R^{e} is —NH₂, —OH, or 3-15 membered heterocyclyl, or
   (i) —S(O)₂—R^{f}, wherein R^{f} is C₁₋₆alkyl or 3-15 membered heterocyclyl,
(ii) C₃₋₁₀cycloalkyl, wherein the C₃₋₁₀cycloalkyl of R² is optionally substituted with one or more R^{q}, wherein R^{q} is 5-20 membered heteroaryl or C₆₋₂₀aryl, wherein the C₆₋₂₀aryl of R^{q} is optionally substituted with one or more C₁₋₆alkoxy,
(iii) 3-15 membered heterocyclyl, wherein the 3-15 membered heterocyclyl of R² is optionally substituted with one or more halo, oxo, C₁₋₆alkyl, —C(O)—C₁₋₆alkyl, or 5-20 membered heteroaryl,
(iv) 5-20 membered heteroaryl or —(C₁₋₄alkyl)(5-20 membered heteroaryl), wherein the C₁₋₄ alkyl is optionally substituted with one or more or more —OH, halo, —NH₂, —NH(C₁₋₆alkyl), —N(C₁₋₆alkyl)₂, and wherein the 5-20 membered heteroaryl of the 5-20 membered heteroaryl or —(C₁₋₄alkyl)(5-20 membered heteroaryl), is optionally substituted with one or more R^{s}, wherein
   R^{s} is halo, C₁₋₆alkyl, C₁₋₆alkoxy, —NH₂, —NH(C₁₋₆alkyl), —N(C₁₋₆alkyl)₂, —NH—C(O)—C₁₋₆alkyl, C₆₋₂₀aryl, C₃₋₁₀cycloalkyl, 3-15 membered heterocyclyl, 5-20 membered heteroaryl, or —C(O)—C₁₋₆alkoxy, wherein
      the C₁₋₆alkyl of R^{s} is optionally substituted with one or more halo, C₁₋₆alkoxy, —NH₂, —NH(C₁₋₆alkyl), —N(C₁₋₆alkyl)₂, —NH—C(O)C₁₋₆alkyl, or —NH—C(O)—C₁₋₆alkoxy, and
      the 3-15-membered heterocyclyl of R^{s} is optionally substituted with one or more halo or —C(O)—C₁₋₆alkoxy,
(v) —N(R^{g})(R^{h}), wherein R^{g} and R^{h} are independently H or C₁₋₆alkyl,
(vi) —C(O)—R^{j}, wherein Ri is C₃₋₁₀cycloalkyl, —NH(C₁₋₆alkyl), —N(C₁₋₆alkyl)₂, or —NH(5-20 membered heteroaryl), and
(vii) C₆₋₂₀aryl, wherein the C₆₋₂₀aryl of R² is optionally substituted with one or more 5-20 membered heteroaryl or —O—R^{p}, wherein R^{p} is 3-15 membered heterocyclyl, wherein the 3-15 membered heterocyclyl of R^{p} is optionally substituted with one or more —C(O)—C₁₋₆alkyl; and
each RG is independently a reactive group suitable for attaching L-1 to the compound of formula (I-1B);
   to form a compound of formula (I), such as a compound of formula (II).

In some embodiments, each RG is independently selected from the group consisting of carboxylic acid or hydroxy groups to form amides, and sulfonic acid, groups to form sulfonamides. In some embodiments, RG is a carboxylic acid. In some embodiments, RG is a sulfonic acid.

In some embodiments, the process further comprises the presence of a coupling or condensation reagent. In some embodiments, the condensation reagent is TCFH-NMI.

In some embodiments, a process for preparing a compound of formula (I), or a stereoisomer or tautomer thereof, or a pharmaceutically acceptable salt of any of the foregoing, comprises reacting a compound of formula (I-2B):

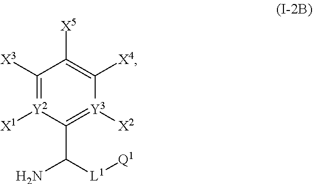

wherein,
Y² and Y³ are each C, or
one of Y² and Y³ is N and the other of Y² and Y³ is C;
X¹ and X² are each independently H, C₁₋₆alkyl, or C₁₋₆alkoxy;
X³ and X⁴ are each independently H, halo, C₁₋₆alkyl, C₁₋₆alkoxy, or 5-20 membered heteroaryl, wherein the C₁₋₆alkyl of X³ and X⁴ is optionally substituted with one of more halo;
X⁵ is H, C₁₋₆alkyl, C₁₋₆alkoxy, or C₃₋₁₀cycloalkyl;
either
(1) L¹ is absent; and
Q¹ is selected from (i) to (iv):
(i) phenyl, wherein the phenyl of Q¹ is substituted with one or more halo, C₁₋₆alkyl, C₂₋₆alkenyl, —NH₂, —NH—C(O)—(C₁₋₆alkyl), —NH—C(O)-(3-15 membered heterocyclyl), C₃₋₁₀cycloalkyl, or 5-20 membered heteroaryl, wherein
   the C₁₋₆alkyl is optionally substituted with one or more halo, —NH—C(O)—NH(C₁₋₆alkyl), —NH—C(O)—C₁₋₆alkyl, or —NH—C(O)—C₁₋₆alkoxy,
   the C₃₋₁₀cycloalkyl is optionally substituted with one or more halo or C₁₋₆alkyl, and
   the 5-20 membered heteroaryl is optionally substituted with one or more C₁₋₆alkyl,
(ii) 3-15 membered heterocyclyl, wherein the 3-15 membered heterocyclyl of Q¹ is optionally substituted with one or more oxo, or C₁₋₆alkyl,
(iii) 5-20 membered heteroaryl, wherein the 5-20 membered heteroaryl of Q¹ is optionally substituted with one or more halo, C₁₋₆alkyl, C₂₋₆alkenyl, C₁₋₆alkoxy, —NH₂, or C₃₋₁₀cycloalkyl, wherein,
   the C₁₋₆alkyl is optionally substituted with one or more halo, and
   the C₃₋₁₀cycloalkyl is optionally substituted with one or more halo or C₁₋₆alkyl, and
(iv) C₃₋₁₀cycloalkyl;
or
(2) L¹ is —CH₂—; and
Q¹ is C₃₋₁₀cycloalkyl;
   with a compound of formula (L-1):

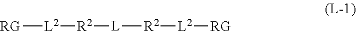

wherein
L² is —C(O)— or —S(O)₂—;
R² is selected from (i) to (vii):
(i) C₁₋₆alkyl, wherein the C₁₋₆alkyl of R² is optionally substituted with one or more R^{a}, wherein R^{a} is:
   (a) —OH,
   (b) cyano,
   (c) C₂₋₆alkynyl,
   (d) C₆₋₂₀aryl, wherein the C₆₋₂₀aryl of R^{a} is optionally substituted with one or more halo, cyano, C₁₋₆alkoxy, or —NH—C(O)—C₁₋₆alkyl,
   (e) 3-15 membered heterocyclyl, wherein the 3-15 membered heterocyclyl of R^{a} is optionally substituted with one or more R^{c}, wherein
      R^{c} is halo, oxo, C₁₋₆alkyl, C₁₋₆alkoxy, —C(O)—C₁₋₆alkyl, or —C(O)—C₁₋₆alkoxy, wherein
         the C₁₋₆alkyl of R^{c} is optionally substituted with one or more halo or C₂₋₆alkynyl, and
         the —C(O)—C₁₋₆alkoxy of R^{c} is optionally substituted with one or more halo,
   (f) —N(R^{c})(R^{d}), wherein R^{c} and R^{d} of —N(R^{c})(R^{d}) are, independently of each other, H, C₁₋₆alkyl, —C(O)—C₁₋₆alkyl, —C(O)—C₁₋₆alkoxy, —C(O)—NH₂, —C(O)—NH(C₁₋₆alkyl), —C(O)—N(C₁₋₆alkyl)₂, —C(O)-(3-15 membered heterocyclyl), —CH₂—C(O)—NH₂, 3-15 membered heterocyclyl, or 5-20 membered heteroaryl, wherein
      the C₁₋₆alkyl of R^{c} or R^{d} is optionally substituted with one or more —C(O)—NH₂,
      the —C(O)—C₁₋₆alkyl of R^{c} or R^{d} is optionally substituted with one or more halo,
      the 3-15 membered heterocyclyl and the 5-20 membered heteroaryl of R^{c} or R^{d} are independently optionally substituted with one or more C₁₋₆alkyl,
      the —C(O)-(3-15 membered heterocyclyl) of R^{c} or R^{d} is optionally substituted with one or more halo, —C(O)—C₁₋₆alkoxy, or C₁₋₆alkyl, wherein the C₁₋₆alkyl is optionally substituted with one or more halo, C₁₋₆alkoxy, or C₃₋₁₀cycloalkyl, and
      the C₁₋₆alkyl of the —C(O)—N(C₁₋₆alkyl)₂ of R^{c} or R^{d} are, independently of each other, optionally substituted with one or more halo or C₆₋₂₀aryl,
   (g) —O—R^{e}, wherein R^{e} is C₁₋₆alkyl, C₆₋₂₀aryl, —C(O)-(3-15 membered heterocyclyl), —C(O)—N—(C₁₋₆alkyl)₂, or 5-20 membered heteroaryl, wherein
      the C₁₋₆alkyl of R^{e} is optionally substituted with one or more C₁₋₆alkoxy, wherein the C₁₋₆alkoxy is optionally substituted with one or more C₂₋₆alkynyl,
      the C₆₋₂₀aryl of R^{e} is optionally substituted with one or more C₁₋₆alkyl, and
      the —C(O)-(3-15 membered heterocyclyl) of R^{e} is optionally substituted with one or more C₁₋₆alkyl, C₁₋₆alkoxy, or —C(O)—C₁₋₆alkoxy, wherein the C₁₋₆alkyl is optionally substituted with one or more halo, C₁₋₆alkoxy, or C₃₋₁₀cycloalkyl,
(h) —C(O)—R^{e}, wherein R^{e} is —NH₂, —OH, or 3-15 membered heterocyclyl, or
(i) —S(O)₂—R^{f}, wherein R^{f} is C₁₋₆alkyl or 3-15 membered heterocyclyl,
(ii) C₃₋₁₀cycloalkyl, wherein the C₃₋₁₀cycloalkyl of R² is optionally substituted with one or more R^{q}, wherein R^{q} is 5-20 membered heteroaryl or C₆₋₂₀aryl, wherein the C₆₋₂₀aryl of R^{q} is optionally substituted with one or more C₁₋₆alkoxy,
(iii) 3-15 membered heterocyclyl, wherein the 3-15 membered heterocyclyl of R² is optionally substituted with one or more halo, oxo, C₁₋₆alkyl, —C(O)—C₁₋₆alkyl, or 5-20 membered heteroaryl,
(iv) 5-20 membered heteroaryl or —(C₁₋₄alkyl)(5-20 membered heteroaryl), wherein the C₁₋₄ alkyl is optionally substituted with one or more or more —OH, halo, —NH₂, —NH(C₁₋₆alkyl), —N(C₁₋₆alkyl)₂, and wherein the 5-20 membered heteroaryl of the 5-20 membered heteroaryl or —(C₁₋₄alkyl)(5-20 membered heteroaryl), is optionally substituted with one or more R^{s}, wherein
   R^{s} is halo, C₁₋₆alkyl, C₁₋₆alkoxy, —NH₂, —NH(C₁₋₆alkyl), —N(C₁₋₆alkyl)₂, —NH—C(O)—C₁₋₆alkyl, C₆₋₂₀aryl, C₃₋₁₀cycloalkyl, 3-15 membered heterocyclyl, 5-20 membered heteroaryl, or —C(O)—C₁₋₆alkoxy, wherein
      the C₁₋₆alkyl of R^{s} is optionally substituted with one or more halo, C₁₋₆alkoxy, —NH₂, —NH(C₁₋₆alkyl), —N(C₁₋₆alkyl)₂, —NH—C(O)C₁₋₆alkyl, or —NH—C(O)—C₁₋₆alkoxy, and
      the 3-15-membered heterocyclyl of R^{s} is optionally substituted with one or more halo or —C(O)—C₁₋₆alkoxy,
(v) —N(R^{g})(R^{h}), wherein R^{g} and R^{h} are independently H or C₁₋₆alkyl,
(vi) —C(O)—R^{j}, wherein Ri is C₃₋₁₀cycloalkyl, —NH(C₁₋₆alkyl), —N(C₁₋₆alkyl)₂, or —NH(5-20 membered heteroaryl), and
(vii) C₆₋₂₀aryl, wherein the C₆₋₂₀aryl of R² is optionally substituted with one or more 5-20 membered heteroaryl or —O—R^{p}, wherein R^{p} is 3-15 membered heterocyclyl, wherein the 3-15 membered heterocyclyl of R^{p} is optionally substituted with one or more —C(O)—C₁₋₆alkyl; and
each RG is independently a reactive group suitable for attaching L-1 to the compound of formula (I-1B);
   to form a compound of formula (I), such as a compound of formula (III).

In some embodiments, each RG is independently selected from the group consisting of carboxylic acid or hydroxy groups to form amides, and sulfonic acid, groups to form sulfonamides.

In some embodiments, RG is a carboxylic acid. In some embodiments, RG is a sulfonic acid.

In some embodiments, the process further comprises the presence of a coupling or condensation reagent. In some embodiments, the condensation reagent is TCFH-NMI.

### ENUMERATED EMBODIMENTS

Embodiment 1. The compound of formula (I), or a stereoisomer or tautomer thereof, or a pharmaceutically acceptable salt of any of the foregoing, wherein the compound is selected from the group consisting of:

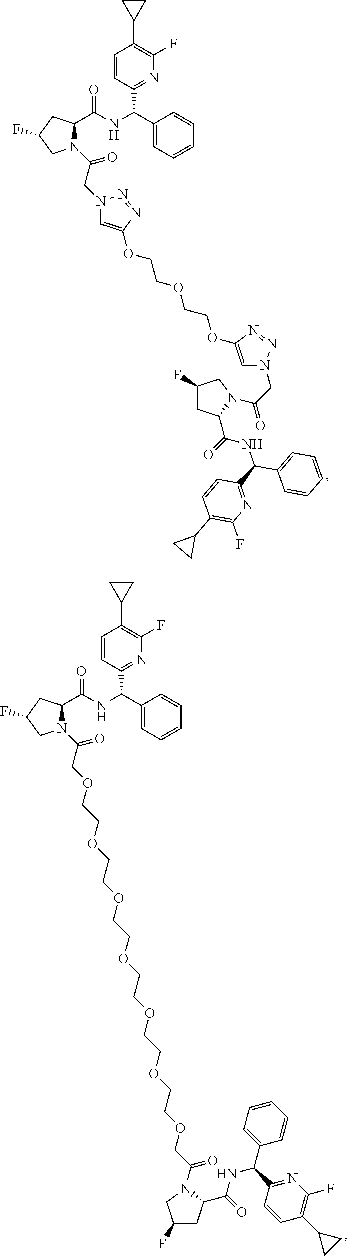 [SMILES: O=C(N[C@@H](c1ccccc1)c1ccc(C2CC2)c(F)n1)[C@@H]1C[C@@H](F)CN1C(=O)Cn1cc(OCCOCCOc2cn(CC(=O)N3C[C@H](F)C[C@H]3C(=O)N[C@@H](c3ccccc3)c3ccc(C4CC4)c(F)n3)nn2)nn1 ; O=C(N[C@@H](c1ccccc1)c1ccc(C2CC2)c(F)n1)[C@@H]1C[C@@H](F)CN1C(=O)COCCOCCOCCOCCOCCOCCOCC(=O)N1C[C@H](F)C[C@H]1C(=O)N[C@@H](c1ccccc1)c1ccc(C2CC2)c(F)n1]

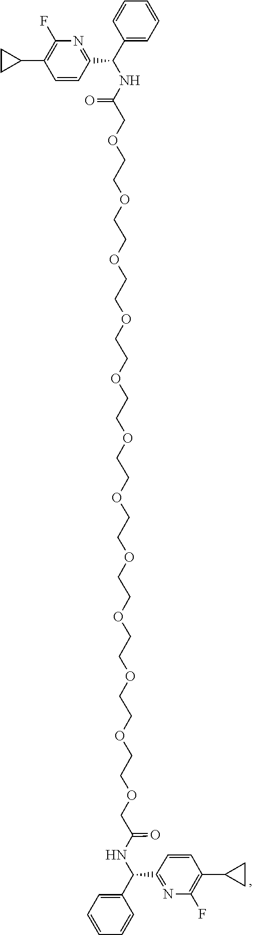 [SMILES: O=C(COCCOCCOCCOCCOCCOCCOCCOCCOCCOCCOCCOCC(=O)N[C@@H](c1ccccc1)c1ccc(C2CC2)c(F)n1)N[C@@H](c1ccccc1)c1ccc(C2CC2)c(F)n1]

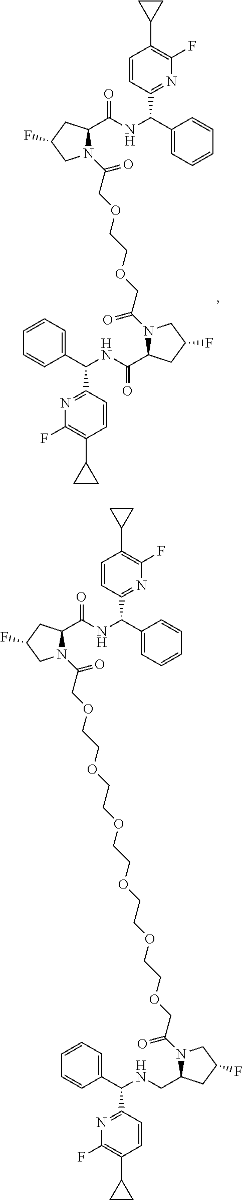

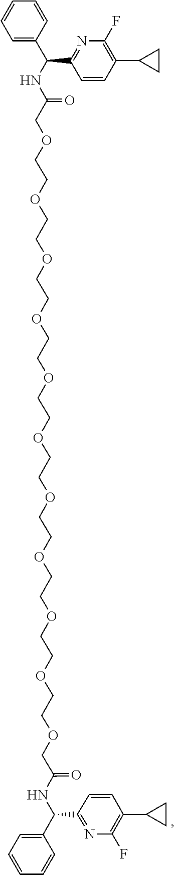 [SMILES: O=C(COCCOCCOCCOCCOCCOCCOCCOCCOCCOCCOCC(=O)N[C@@H](c1ccccc1)c1ccc(C2CC2)c(F)n1)N[C@@H](c1ccccc1)c1ccc(C2CC2)c(F)n1]

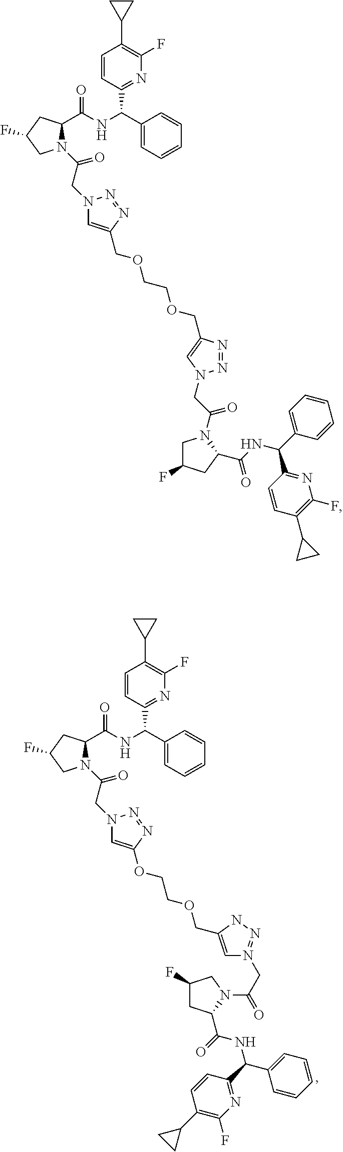

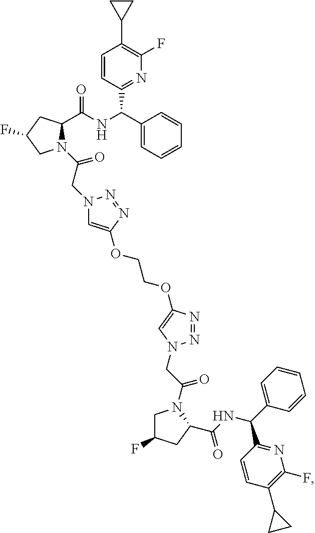 [SMILES: O=C(N[C@@H](c1ccccc1)c1ccc(C2CC2)c(F)n1)[C@@H]1C[C@@H](F)CN1C(=O)Cn1cc(OCCOc2cn(CC(=O)N3C[C@H](F)C[C@H]3C(=O)N[C@@H](c3ccccc3)c3ccc(C4CC4)c(F)n3)nn2)nn1]

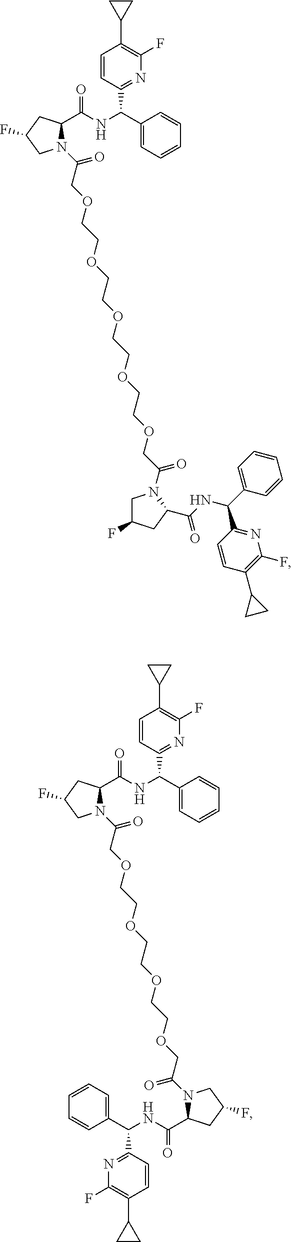 [SMILES: O=C(N[C@@H](c1ccccc1)c1ccc(C2CC2)c(F)n1)[C@@H]1C[C@@H](F)CN1C(=O)COCCOCCOCCOCCOCC(=O)N1C[C@H](F)C[C@H]1C(=O)N[C@@H](c1ccccc1)c1ccc(C2CC2)c(F)n1 ; O=C(N[C@@H](c1ccccc1)c1ccc(C2CC2)c(F)n1)[C@@H]1C[C@@H](F)CN1C(=O)COCCOCCOCCOCC(=O)N1C[C@H](F)C[C@H]1C(=O)N[C@@H](c1ccccc1)c1ccc(C2CC2)c(F)n1]

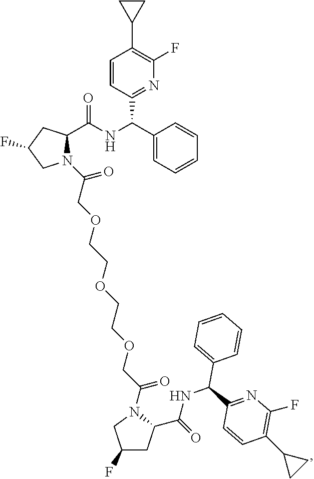 [SMILES: O=C(N[C@@H](c1ccccc1)c1ccc(C2CC2)c(F)n1)[C@@H]1C[C@@H](F)CN1C(=O)COCCOCCOCC(=O)N1C[C@H](F)C[C@H]1C(=O)N[C@@H](c1ccccc1)c1ccc(C2CC2)c(F)n1]

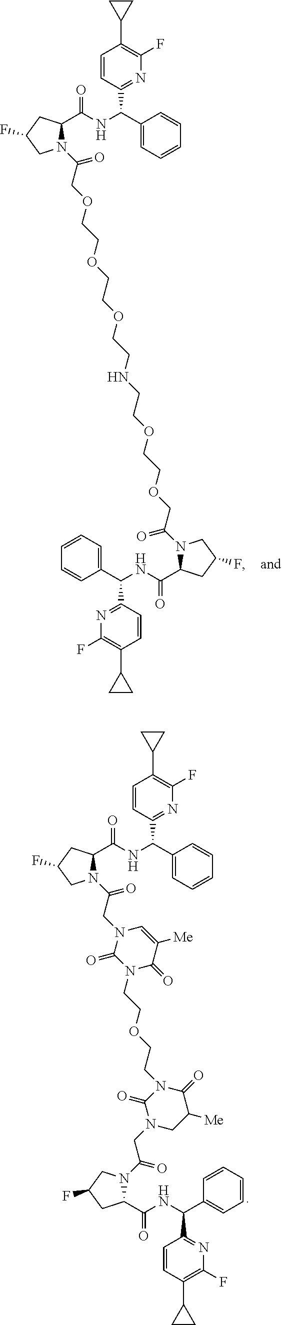 [SMILES: O=C(N[C@@H](c1ccccc1)c1ccc(C2CC2)c(F)n1)[C@@H]1C[C@@H](F)CN1C(=O)COCCOCCNCCOCCOCCOCC(=O)N1C[C@H](F)C[C@H]1C(=O)N[C@@H](c1ccccc1)c1ccc(C2CC2)c(F)n1 ; Cc1cn(CC(=O)N2C[C@H](F)C[C@H]2C(=O)N[C@@H](c2ccccc2)c2ccc(C3CC3)c(F)n2)c(=O)n(CCOCCN2C(=O)C(C)CN(CC(=O)N3C[C@H](F)C[C@H]3C(=O)N[C@@H](c3ccccc3)c3ccc(C4CC4)c(F)n3)C2=O)c1=O]

### EXAMPLES

The following synthetic reaction schemes, which are detailed in the Schemes and Examples, are merely illustrative of some of the methods by which the compounds of the present disclosure, or an embodiment or aspect thereof, can be synthesized. Various modifications to these synthetic reaction schemes can be made, as will be apparent to those of ordinary skill in the art.

The starting materials and the intermediates of the synthetic reaction schemes can be isolated and purified if desired using conventional techniques, including, but not limited to, filtration, distillation, crystallization, chromatography, and the like. Such materials can be characterized using conventional means, including physical constants and spectral data.

Although certain exemplary embodiments are depicted and described herein, the compounds of the present disclosure, or any variation or embodiment thereof, may be prepared using appropriate starting materials according to the methods described generally herein and/or by methods available to one of ordinary skill in the art.

### SYNTHETIC EXAMPLES

As depicted in the Schemes and Examples below, in certain exemplary embodiments, compounds of formula (I), or any variation or embodiment thereof, as described elsewhere herein, or a stereoisomer or tautomer thereof, or a pharmaceutically acceptable salt of any of the foregoing, are prepared according to the general procedures. The general methods below, and other methods known to synthetic chemists of ordinary skill in the art, can be applied to all formulae, variations, embodiments, and species described herein.

### Schemes

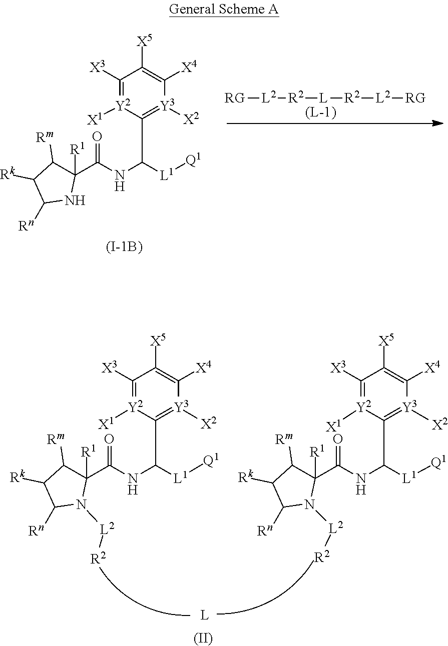

Compounds of the formula (I), such as compounds of formula (II) may be prepared according to the general synthetic scheme outlined in General Scheme A. Compounds of formula (I-1B), wherein R^{k}, R^{m}, Rⁿ, R¹, X¹, X², X³, X⁴, X⁵, Y², Y³, L¹, and Q¹ are as defined for a compound of formula (I), are coupled with compounds of formula (L-1), wherein each L² and R² are independently as defined for a compound of formula (I), and wherein each RG is independently a reactive group suitable for attaching L-1 to the compound of formula (I-1B). Compounds of formula (I-1B) are processed to compounds of formula (II) using methods outlined in Schemes 1-18 and the Examples below. In some embodiments, compounds of formula (I-1B) are compounds of formula S1-8, S2-10, S3-11, S4-15, S5-12, S6-17, S7-10, S8-12, S9-13, S11-9, or S13-2.

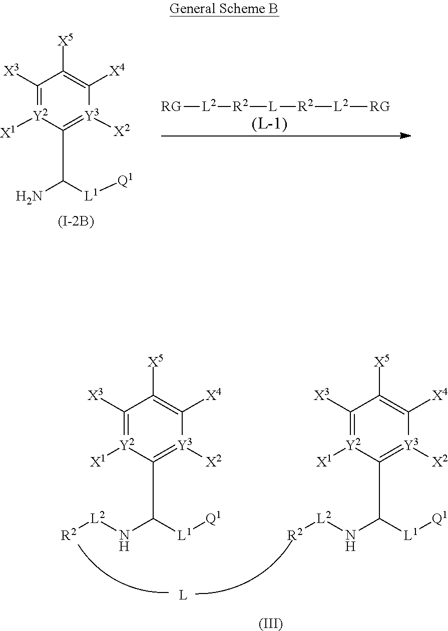

Compounds of the formula (I), such as compounds of formula (III) may be prepared according to the general synthetic scheme outlined in General Scheme B. Compounds of formula (I-2B), wherein X¹, X², X³, X⁴, X⁵, Y², Y³, L¹, and Q¹ are as defined for a compound of formula (I), are coupled with compounds of formula (L-1), wherein each L² and R² are independently as defined for a compound of formula (I), and wherein each RG is independently a reactive group suitable for attaching L-1 to the compound of formula (I-1). Compounds of formula (I-2B) are processed to compounds of formula (II) using methods outlined in Schemes 1-18 and the Examples below. In some embodiments, compounds of formula (I-2B) are compounds of formula S1-5, S2-7, S3-8, S4-12, S5-7, S6-14, S7-5, S8-9, S9-10, S10-5, S11-6, S12-4, S14-4, or S15-4.

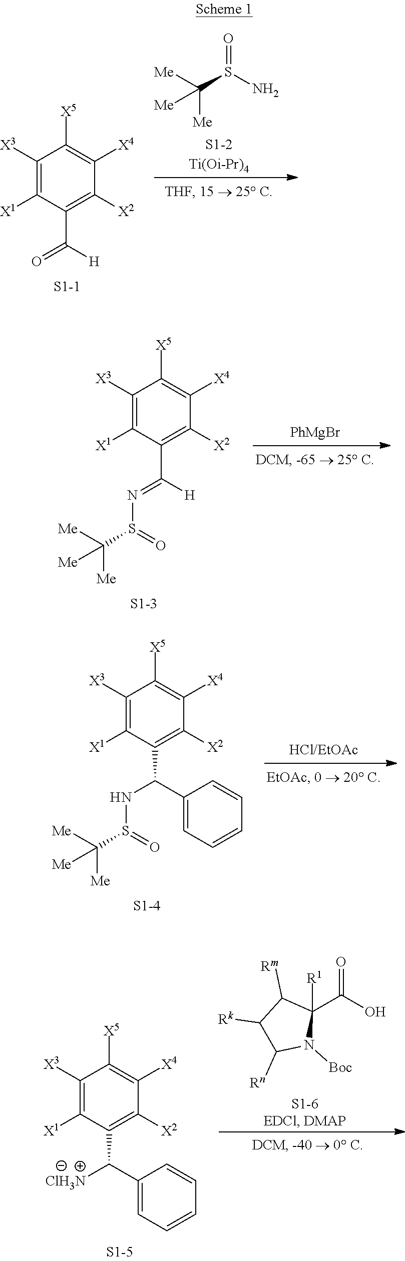

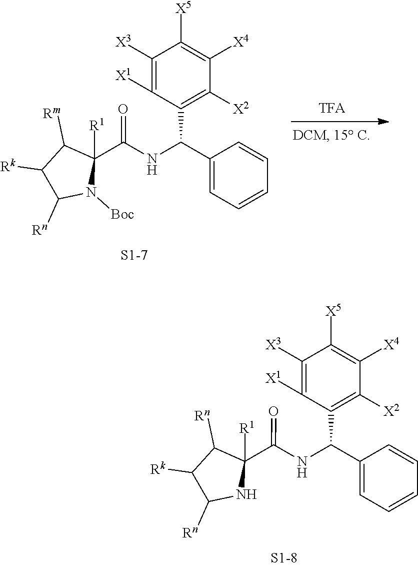

Compounds of the formula S1-8 may be prepared according to the general synthetic scheme outlined in Scheme 1.

Condensation of a chiral sulfinamide such as S1-2 with an aldehyde such as S1-1 provides sulfinimine S1-3. Addition of a reagent such as phenylmagnesium bromide at low temperature, followed by warming to ambient temperature provides benzhydryl sulfinamide S1-4. Sulfinamide S1-3 can be converted to the corresponding amine hydrochloride salt upon treatment with HCl in a solvent such as EtOAc. Amide bond formation between S1-5 and a substituted proline analog such as S1-6 may be achieved with a carbodiimide reagent such as EDCI and DMAP as a catalyst. Removal of the N-Boc group of S1-7 via treatment with a protic acid such as trifluoroacetic acid gives rise to amines such as S1-8.

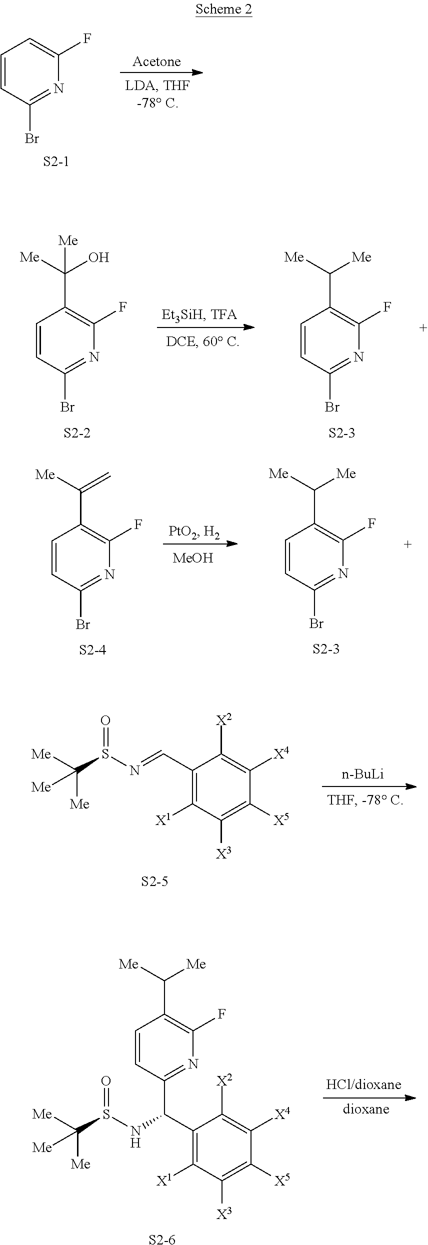

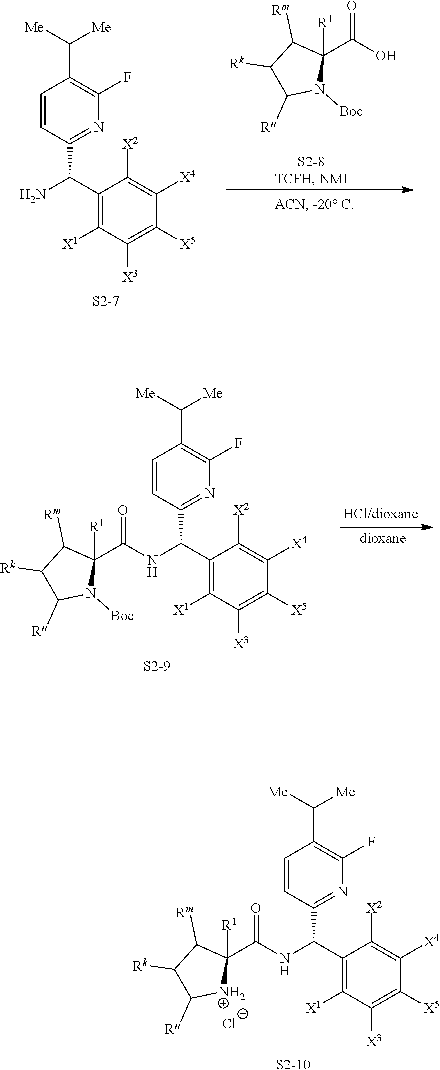

Compounds of the formula S2-10 may be prepared according to the general synthetic scheme outlined in Scheme 2.

Directed ortho-metalation of pyridine S2-1 with an amide base such as LDA in an aprotic solvent such as THF at −78° C. followed by reaction with a ketone such as acetone can generate pyridine S2-2. Treatment of S2-2 with a reducing agent such as triethyl silane and a protic acid such as trifluoroacetic acid generates a mixture of compounds, S2-3 and S2-4. This mixture can be converted to S2-3 by reduction with hydrogen gas and a metal catalyst such as PtO₂. Metal-halogen exchange can be affected by treatment of S2-3 at −78° C. with n-butyllithium, and the pyridyllithium intermediate may then be reacted with sulfinimine S2-5 to generate S2-6. Treatment with a protic acid such as HCl generates the amine S2-7. Coupling of amine S2-7 with proline derivative S2-8 using TCFH and N-methylimidazole base gives rise to S2-9. Removal of the proline N-Boc group by treatment with a protic acid such as HCl, in an aprotic solvent such as 1,4-dioxane, provides amine S2-10.

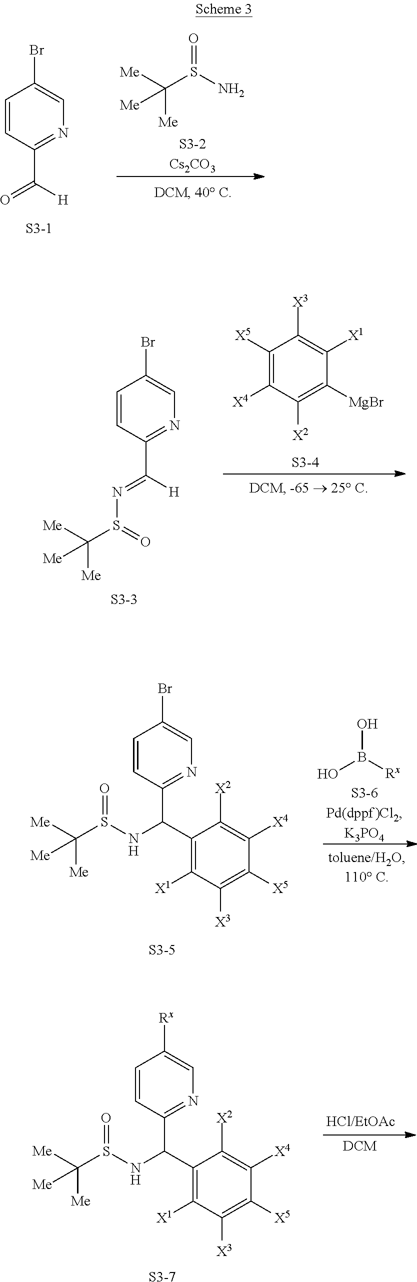

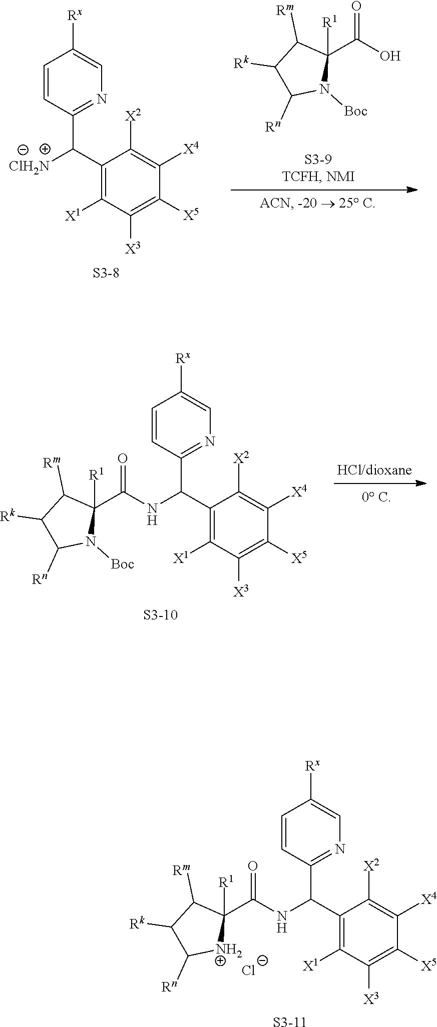

Compounds of the formulae S3-11 may be prepared according to the general synthetic scheme outlined in Scheme 3.

Condensation of racemic sulfinamide S3-2 with pyridyl aldehyde S3-1 generates sulfinimine S3-3. Reaction with arylmagnesium bromide S3-4 generates S3-5, as a racemate. A Suzuki cross-coupling of S3-5 with a boronic acid such as S3-6 using a palladium catalyst such as Pd(dppf)Cl₂ and an inorganic base such as K₃PO₄ generates compound S3-7. Treatment of S3-7 with a protic acid such as HCl in an aprotic solvent mixture such as EtOAc/DCM provides amine S3-8. S3-8 may then be coupled with carboxylic acid S3-9 and processed to compounds S3-11 using methods outlined in Scheme 1.

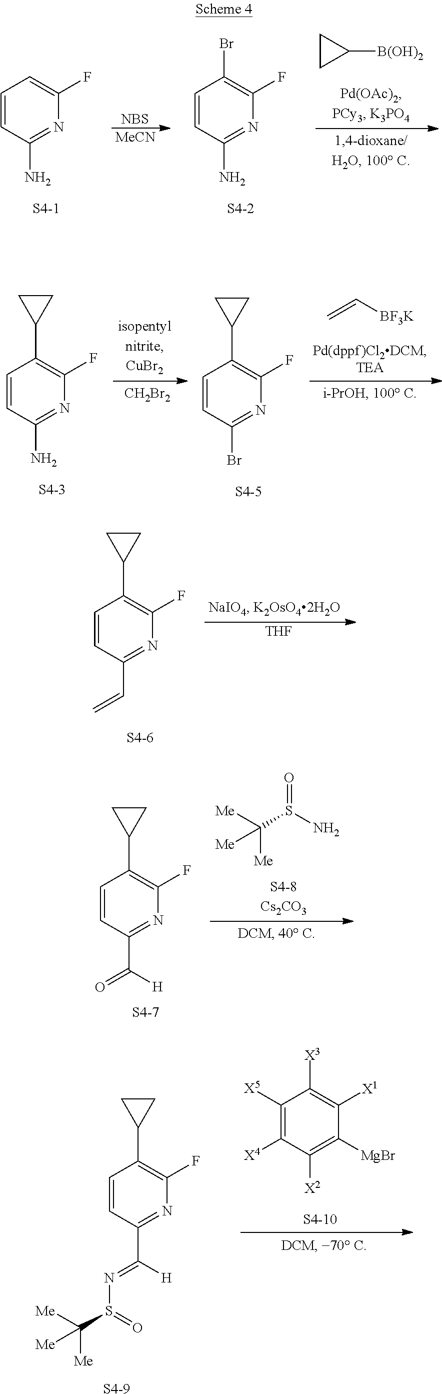

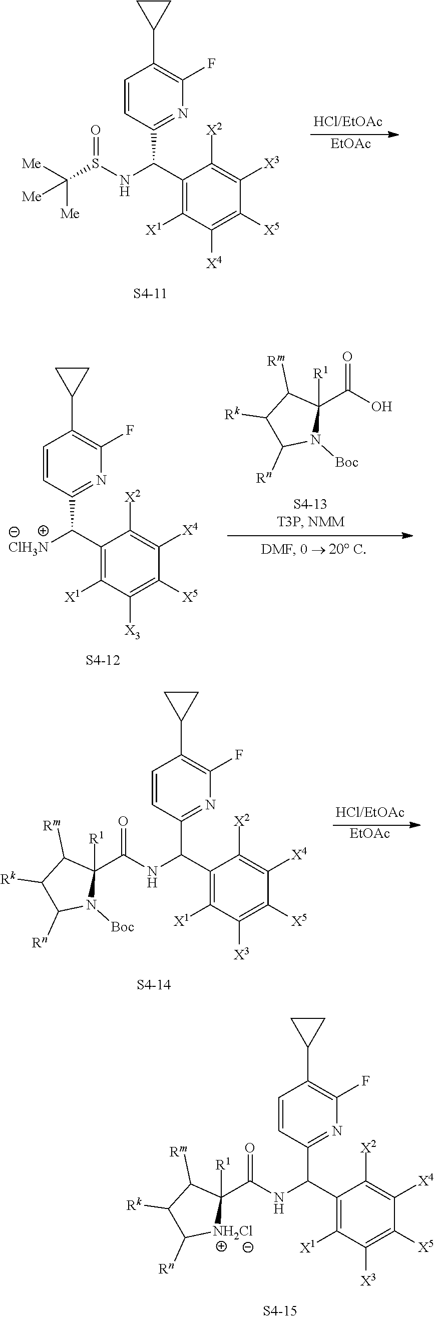

Compounds of the formula S4-15 may be prepared according to the general synthetic scheme outlined in Scheme 4.

Reaction of pyridine S3-1 with an electrophilic brominating agent such as NBS gives pyridine S4-2. Suzuki cross-coupling with cyclopropylboronic acid, using a catalyst such as palladium acetate, a ligand such as tricyclohexyl phosphine, and an inorganic base such K₃PO₄ in a mixed solvent system such as 1,4-dioxane and water, provides S4-3. Conversion of S4-3 to pyridyl bromide S4-5 can be achieved with a Sandmeyer reaction under the action of isopentyl nitrite and cupric bromide in dibromomethane solvent. Suzuki cross coupling of potassium vinyltrifluoroborate with S4-6 using a palladium catalyst such as Pd(dppf)Cl₂ and an inorganic base such as K₃PO₄. Oxidative cleavage of olefin S4-6 with NaIO₄ and K₂OsO₄·2H₂O in a THE generates aldehyde S4-7. Condensation with sulfinamide S4-8 generates sulfinimine S4-9. Reaction of S4-9 with aryl Grignard reagent S4-10 in a solvent such as DCM at low temperature gives rise to S4-11. Cleavage of sulfinamide S4-11 with a HCl in EtOAc generates amine salt S4-12. S4-12 may then be joined with carboxylic acid S4-13 using a coupling agent such as T3P and a base such as NMM in DMF to produce S4-14. Removal of the N-Boc group with HCl in EtOAc generates amine S4-15.

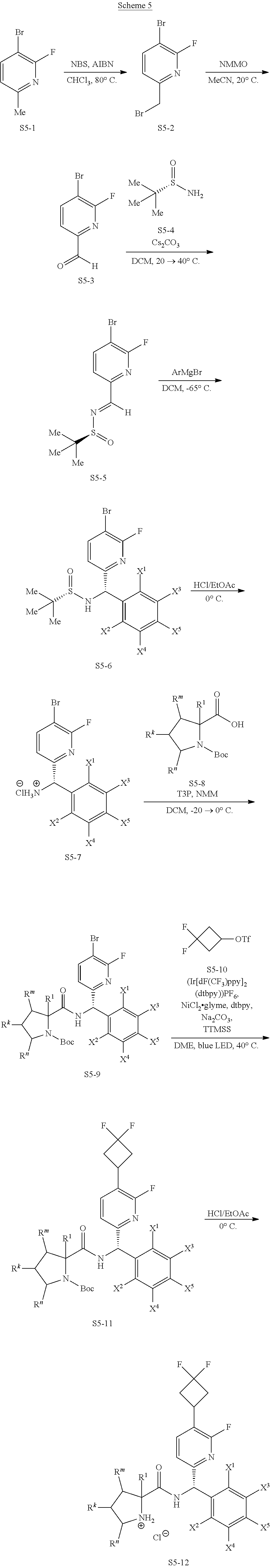

Compounds of the general formula S5-12 can be prepared according to the general scheme outlined in Scheme 5.

Radical bromination of S5-1 with NBS and catalytic AIBN provides S5-2. Oxidation under the action NMMO gives aldehyde S5-3. Condensation of aldehyde with sulfinamide S5-4 using an inorganic base such as cesium carbonate provide sulfinimine S5-5. Addition of an aryl Grignard reagent such as phenylmagnesium bromide at low temperature provides S5-6. Cleavage of the sulfinimide to generate a primary amine salt can be achieved upon treatment with a protic acid such as HCl in a solvent such as EtOAc. Amide bond formation with proline derivative S5-8 proceeds as previously described to give S5-9. Photoredox coupling of triflate S5-10 with S5-9 using an iridium photocatalyst such as (Ir[dF(CF₃)ppy]₂(dtbpy))PF₆, a nickel co-catalyst such as NiCl₂·glyme, a ligand such as 4,4-di-tert-butyl-2,2-bipyridyl, sodium carbonate as base, and tris(trimethylsilyl)silane and blue LED gives cyclobutyl adduct S5-11. Removal of the proline Boc protecting group with HCl in EtOAc, gives compounds of formula S5-12.

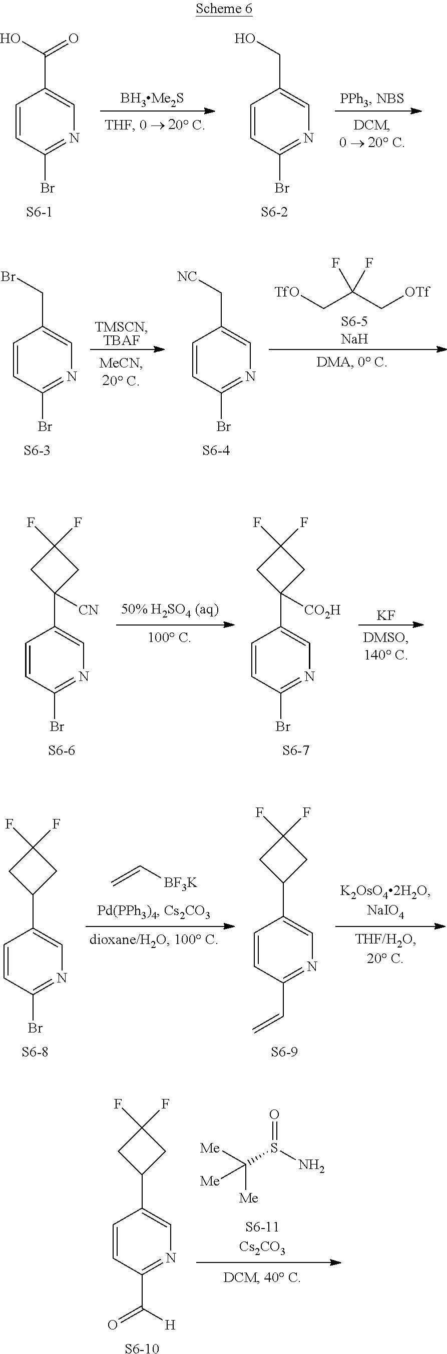

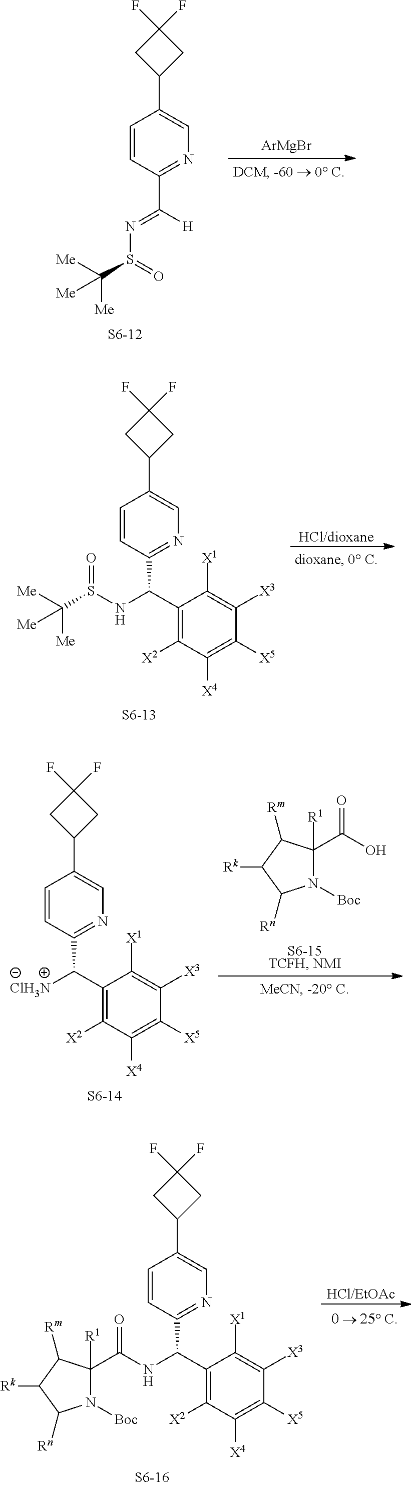

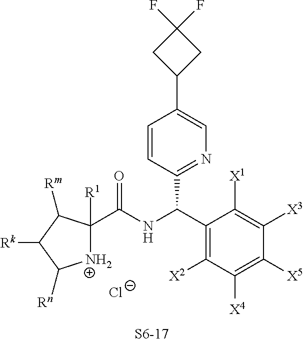

Compounds of the general formula S6-17 can be prepared according to the general scheme outlined in Scheme 6.

Reduction of carboxylic acid S6-1 with borane-methylsulfide complex gives alcohol S6-2. Conversion of S6-2 to alkyl bromide S6-3 is achieved by treatment with triphenylphosphine and NBS in a solvent such as DCM. Selective displacement of the primary bromide can be achieved upon reaction with TMSCN and TBAF in acetonitrile to provide S6-4. Double alkylation of nitrile S6-4 with a di-triflate such as S6-5 gives rise to cyclobutane S6-6. Hydrolysis of the nitrile on treatment with sulfuric acid at elevated temperature gives acid S6-7. Decarboxylation can be achieved by reaction with KF in DMSO at high temperature to provide S6-8. Suzuki-type cross coupling with potassium vinyltrifluoroborate, a palladium catalyst such as tetrakis triphenylphosphine palladium(O) and a base such as cesium carbonate generates S6-9. Lemieux-Johnson oxidation of S6-9 gives aldehyde S6-10, which may then be condensed with sulfinimide S6-11 under conditions previously described to give S6-12. Addition of an aryl Grignard reagent such as phenylmagnesium bromide to S6-12 gives S6-13. Generation of primary amine salt S6-14 may occur upon treatment with HCl in dioxane. Coupling of S6-14 with proline derivative S6-15 using chloro-N,N,N′,N′-tetramethylformamidinium hexafluorophosphate (TCFH) and N-methylimidazole (NMI) gives S6-16. Boc deprotection as described in Scheme 5 gives compounds of formula S6-17.

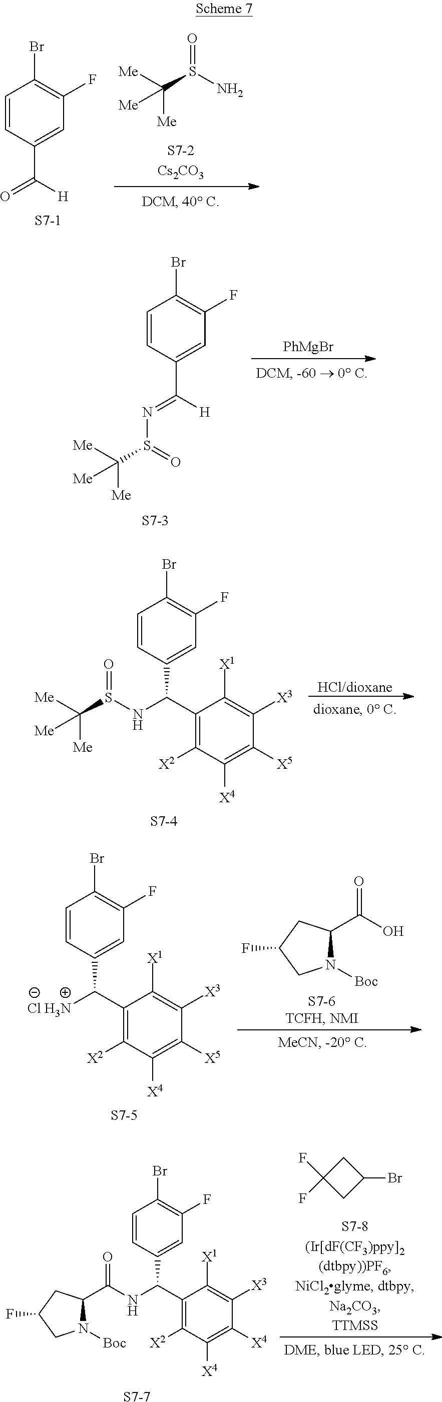

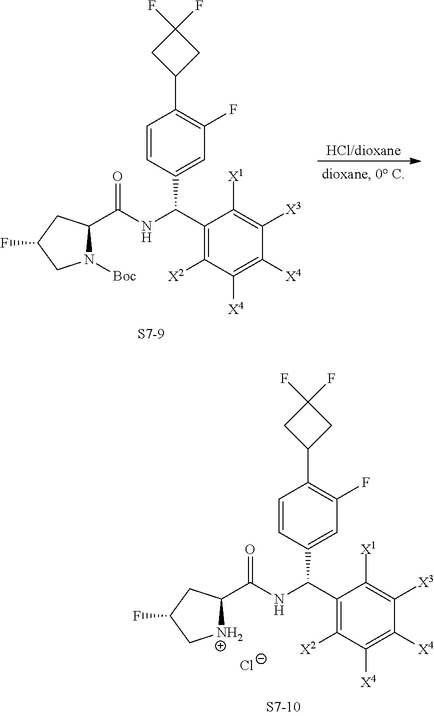

Compounds of the general formulae S7-10 can be prepared according to the general scheme outlined in Scheme 7.

Condensation of sulfinimide S7-2 with aldehyde S7-1 as previously described, followed by addition of an aryl Grignard reagent such as phenylmagnesium bromide, gives S7-4. Generation of the amine salt and coupling with proline derivative S7-6 may be achieved under conditions previously described. Photoredox coupling of cyclobutyl bromide S7-8 under conditions like those described in Scheme 5 gives S7-9. As previously described, Boc deprotection generates compounds of formula S7-10.

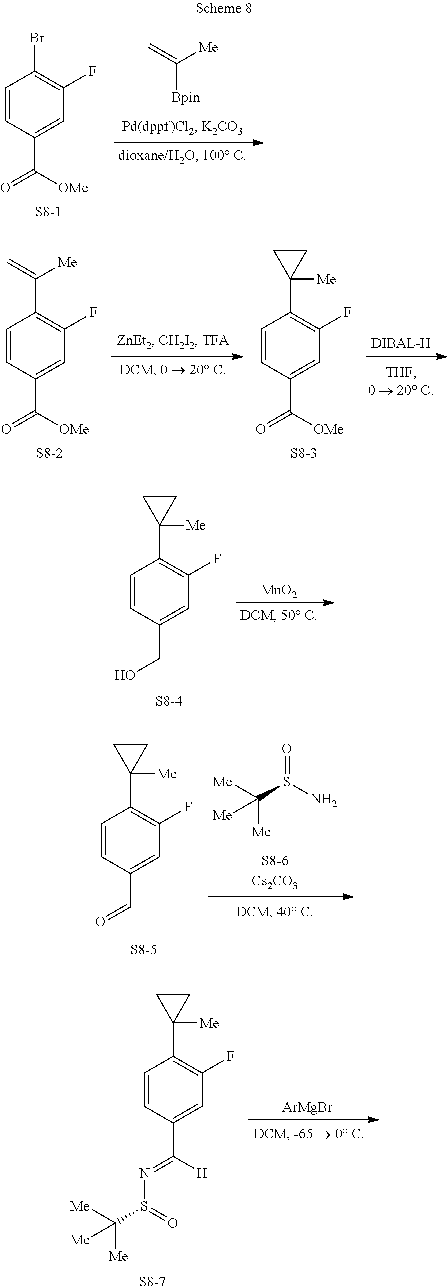

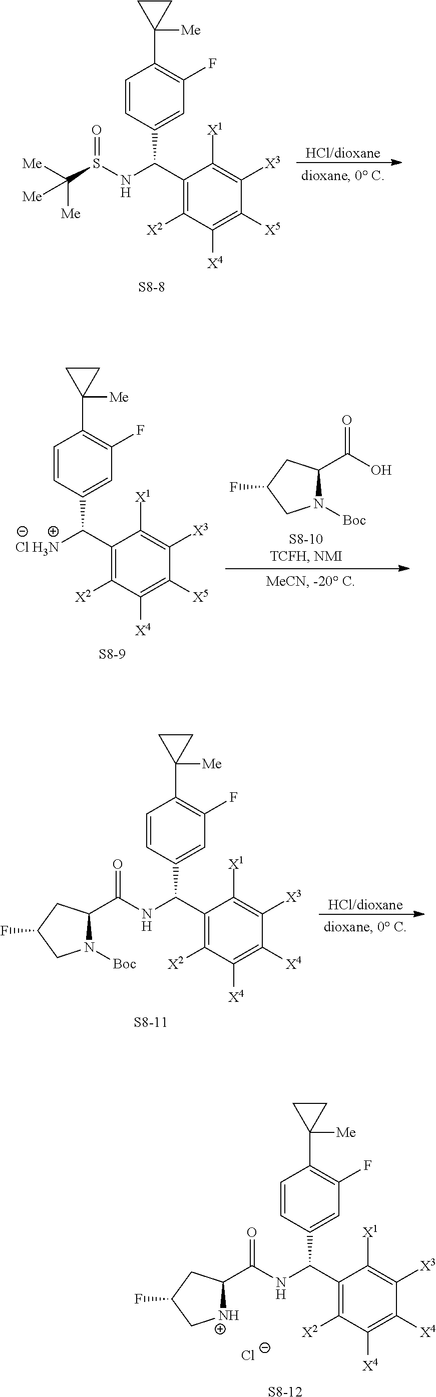

Compounds of the general formula S8-12 can be prepared according to the general scheme outlined in Scheme 8.

An alternative generation of benzhydryl fragments begins with cross-coupling of S8-1 with isopropenylboronic acid pinacol ester with a catalyst such as [1,1′-bis(diphenylphosphino)ferrocene]palladium(II) dichloride and potassium carbonate as base in a mixed dioxane/water solvent at elevated temperature gives S8-2. Simmons-Smith cyclopropanation gives S8-3. Reduction of ester S8-3 with DIBAL-H, followed by oxidation with MnO₂ gives aldehyde S8-5. Aldehyde S8-5 may then be processed over several steps previously described to give compounds of formula S8-12.

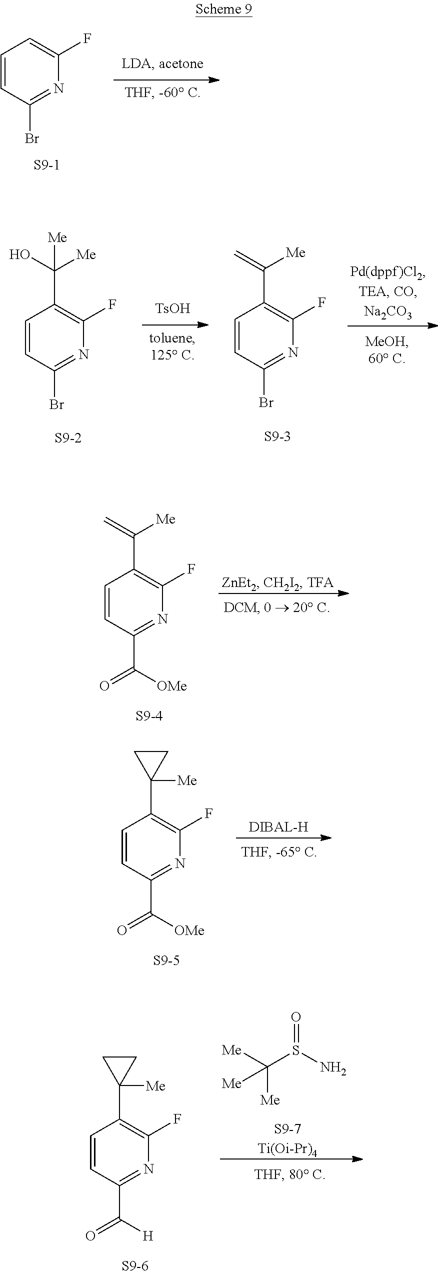

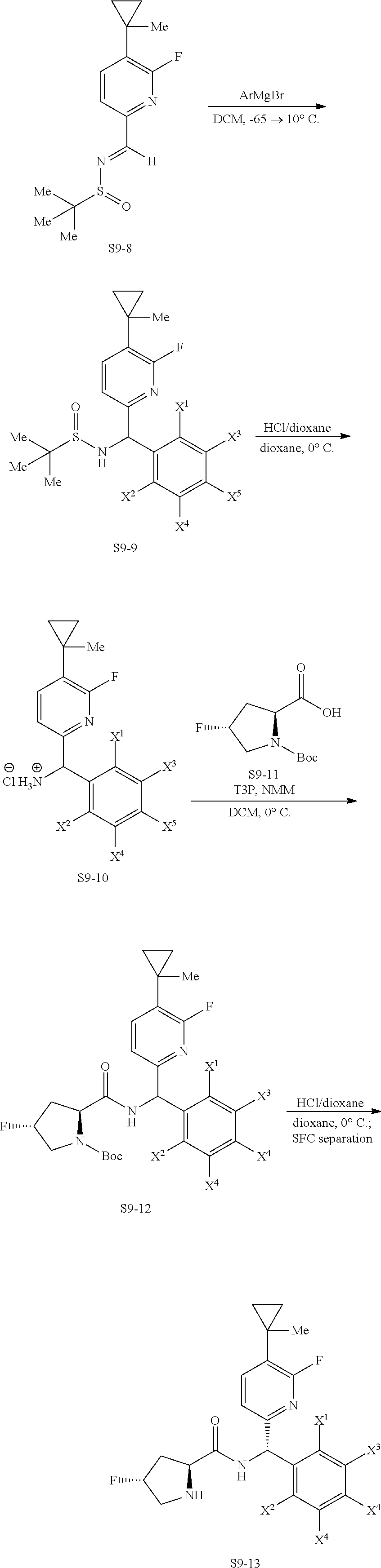

Compounds of the general formula S9-13 can be prepared according to the general scheme outlined in Scheme 9.

An alternative approach to cyclopropane containing analogs begins with directed metalation of S9-1 with LDA and reaction with acetone to generate S9-2. Dehydration under the action of a protic acid such as p-toluene sulfonic acid in toluene at elevated temperature generates olefin S9-3. Cyclopropanation under conditions described in Scheme 8 gives S9-5. Partial reduction of S9-5 to aldehyde S9-6 can be achieved with DIBAL-H in THF at low temperature. Condensation of aldehyde S9-6 with racemic sulfinimide S9-7 gives S9-8. Addition of a Grignard reagent and deprotection under conditions previously described gives amine salt S9-10 as a racemate. Coupling with proline derivative S9-11 under standard conditions and Boc deprotection generates an intermediate which can be further purified by chiral SFC to give amine S9-13 as a single isomer.

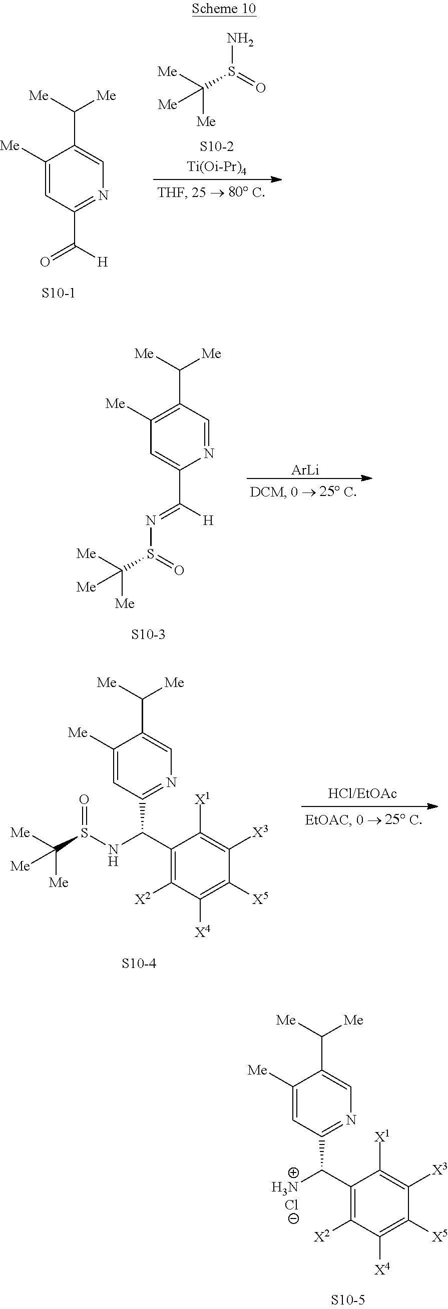

Compounds of the general formula S10-5 can be prepared according to the general scheme outlined in Scheme 10.

An alternative approach to compounds of formula S10-5 starts with condensation of pyridine S10-1 with sulfinimide S10-2. Addition of an aryllithium reagent such as phenyllithium provides S10-4. Deprotection under standard conditions gives amine salts of formula S10-5, which may be further elaborated as described in the Schemes 1-9 and Schemes 11-15.

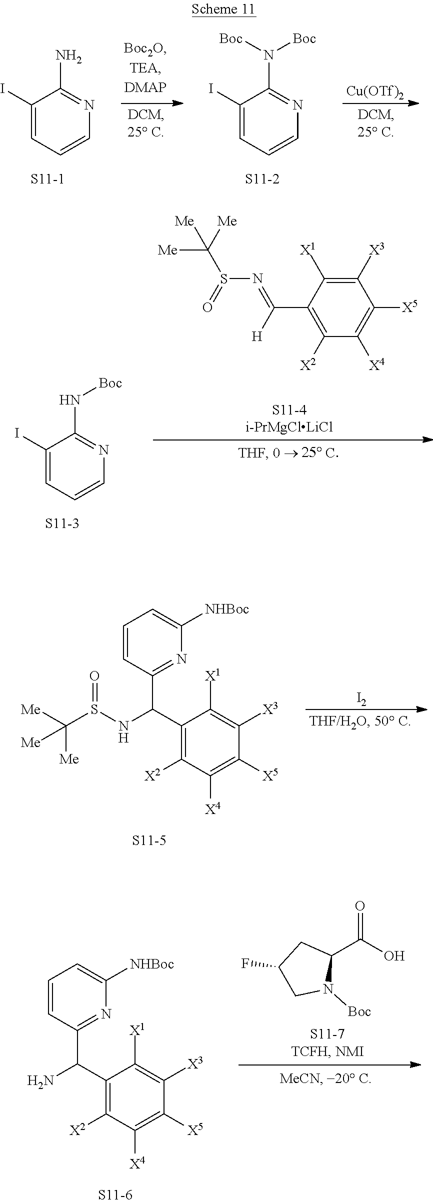

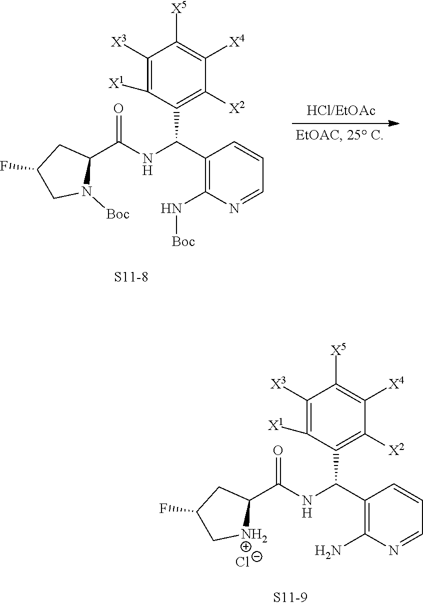

Compounds of the general formula S11-9 can be prepared according to the general scheme outlined in Scheme 11.

Yet another alternative approach to amine intermediates such as S11-6 starts with pyridine S11-1. Bis-carbamate formation, followed by treatment copper (II) triflate gives pyridine S11-3. Conversion of S11-3 to the corresponding Grignard reagent with isopropylmagnesium chloride-lithium chloride complex followed by addition to a racemic sulfinimine such as S11-4 gives S11-5. Selective cleavage of the sulfinamide may occur on reaction with iodine at elevated temperature. Coupling with proline derivative S11-7 and further processing to generate compounds of formula S11-9 occurs as previously described.

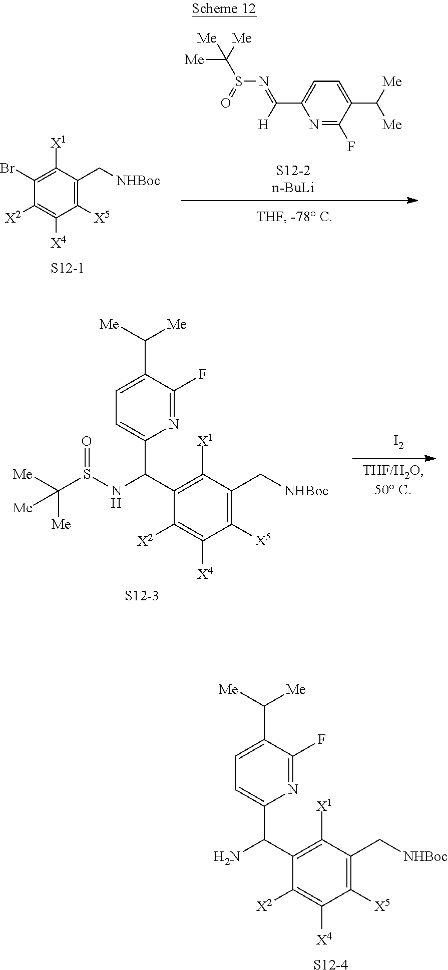

Compounds of the general formula S12-4 can be prepared according to the general scheme outlined in Scheme 12.

An alternative sequence enabling late-stage elaboration of the benzhydryl moieties begins with lithiation of S12-1 and addition of sulfinimine S12-2. Oxidative cleavage with iodine generates S12-4.

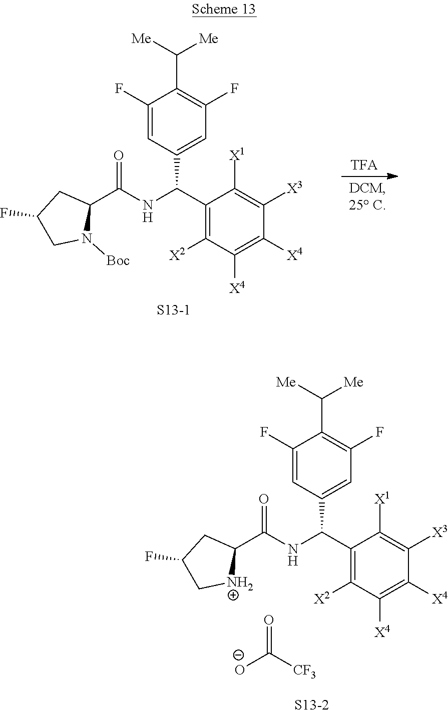

Compounds of the general formula S13-2 can be prepared according to the general scheme outlined in Scheme 13.

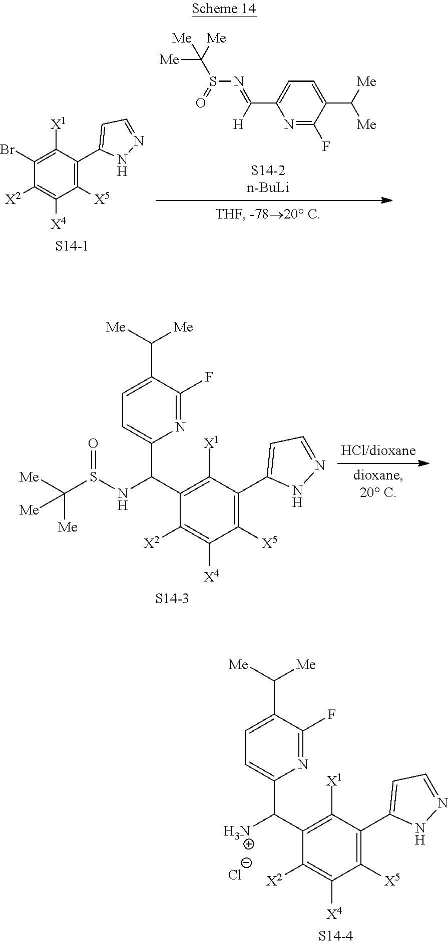

Compounds of the general formulae S14-4 can be prepared according to the general scheme outlined in Scheme 14.

Bromobenzene analogs bearing heterocycles such as pyrazoles can undergo metalation using an excess of n-BuLi and addition to sulfinimines such as S14-2 to give adducts such as S14-3. Conversion to the amine HCl salt can occur under previously described conditions.

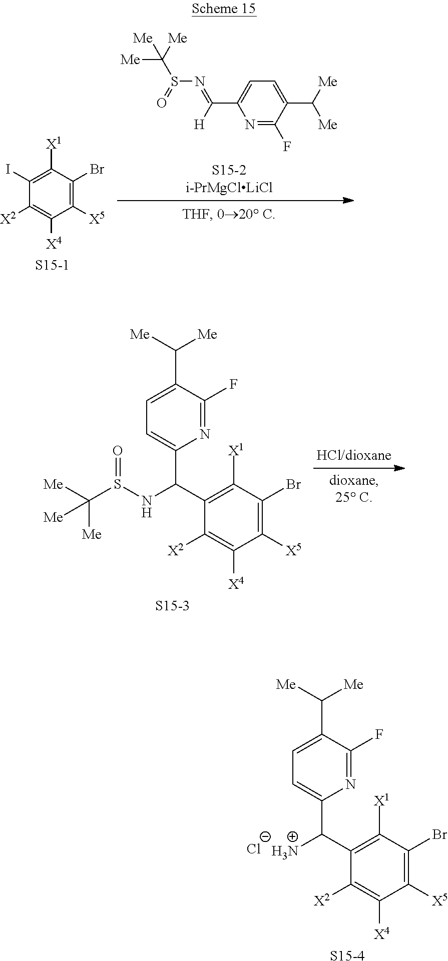

Compounds of the general formula S15-4 can be prepared according to the general scheme outlined in Scheme 15.

An alternative strategy that enables late-stage elaboration to generate compounds of formulae S15-4 begins by selective metal-halogen exchange with S15-1 and addition to sulfinimine S15-2 to generate S15-3. Generation of the amine HCl salt under conditions previously described gives S15-4.

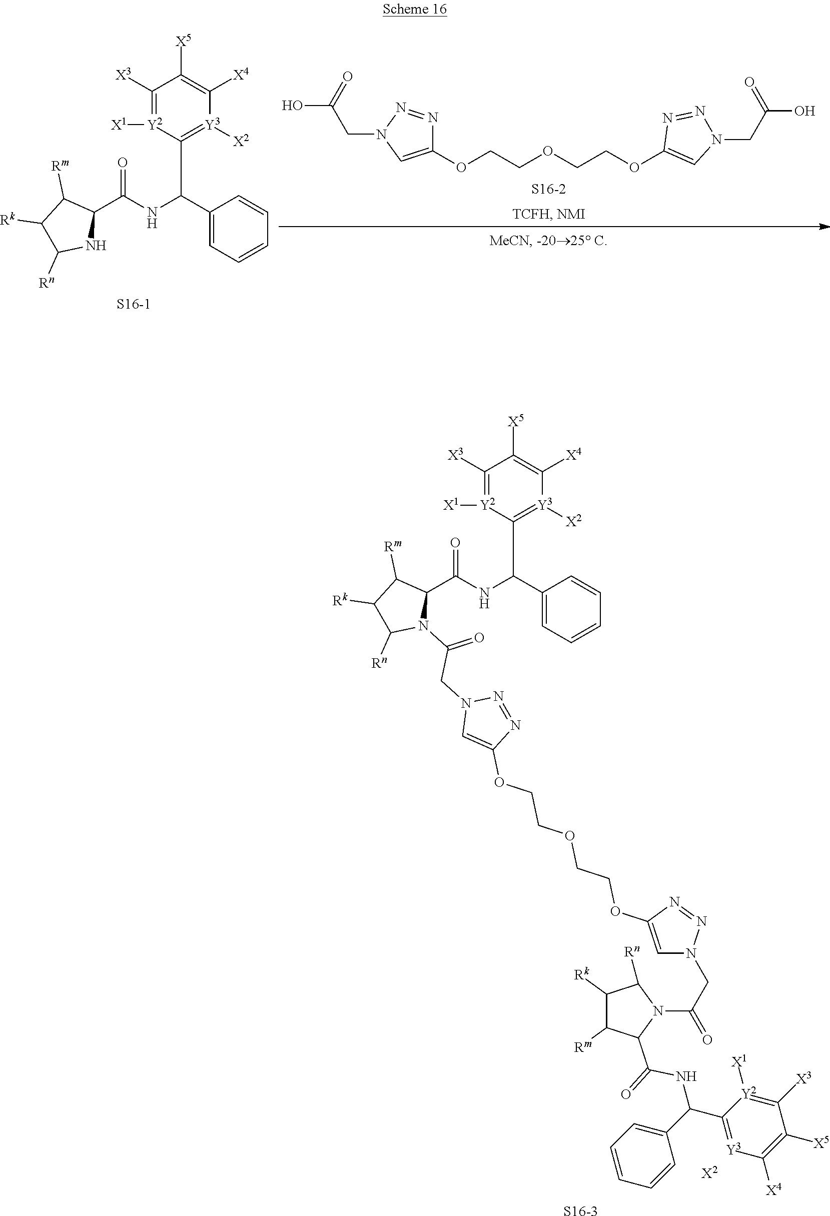

Compounds of the general formula S16-3 can be prepared according to the general scheme outlined in Scheme 16. Coupling of a dicarboxylic acid such as S16-2 with an excess of an amine such as S16-1 in the presence of a coupling reagent such as N,N,N′,N′-tetramethylchloroformamidinium hexafluorophosphate (TFCH) and a base such as N-methylimidazole gives bivalent inhibitors of the formula S16-3.

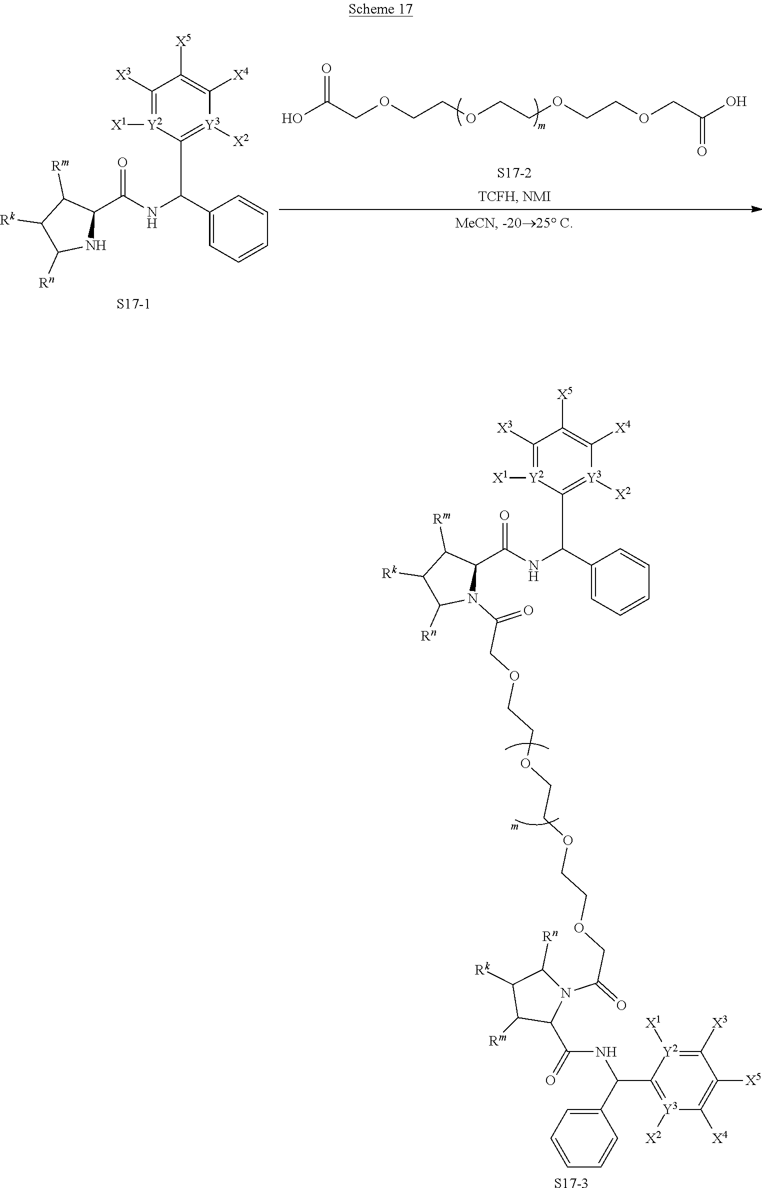

Compounds of the general formula S17-3 can be prepared according to the general scheme outlined in Scheme 17. Coupling of PEG-linked dicarboxylic acid such as S17-2 with an excess of an amine such as S17-1 in the presence of a coupling reagent such as N,N,N′,N′-tetramethylchloroformamidinium hexafluorophosphate (TFCH) and a base such as N-methylimidazole gives bivalent inhibitors of the formula S17-3.

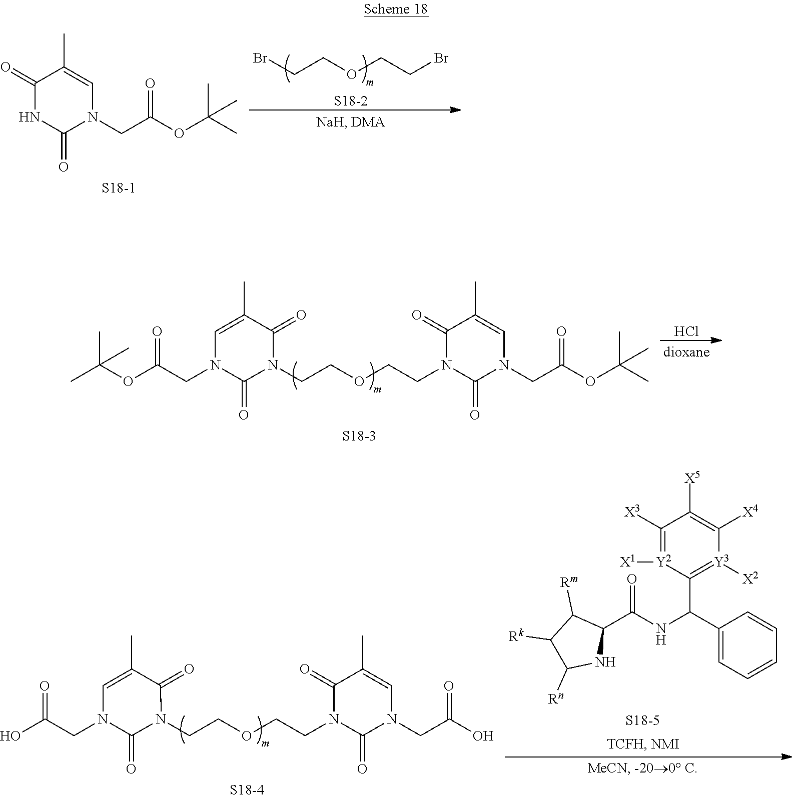

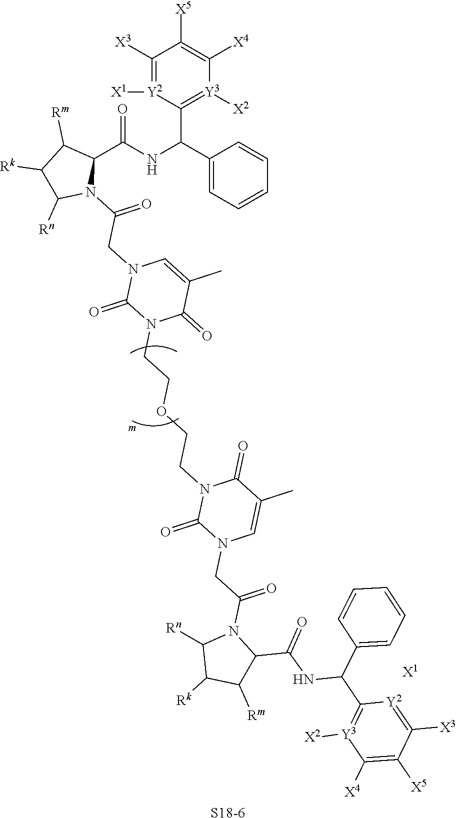

Compounds of the general formula S18-6 can be prepared according to the general scheme outlined in Scheme 18. Alkylation of a heterocyclic compound such as S18-1 with a bis-electrophile such as S18-2 gives S18-3. Removal of the tert-butyl ester protecting groups with HCl in an aprotic solvent such as dioxane gives dicarboxylic acid S18-4. Coupling of S18-4 with an excess of an amine such as S18-5 in the presence of a coupling reagent such as N,N,N′,N′-tetramethylchloroformamidinium hexafluorophosphate (TFCH) and a base such as N-methylimidazole gives bivalent inhibitors of the formula S18-6.

Abbreviations used are those conventional in the art and are in accordance with the Periodic Table of the Elements, CAS version, Handbook of Chemistry and Physics, 75^{th} Ed. The following examples are intended to be illustrative only and not limiting in any way.

| | | | |
|---|---|---|---|
| ° C. | degrees Celsius | K₂CO₃ | potassium carbonate |
| μL | microliter | LDA | lithium diisopropylamide |
| [M + XX]⁺ | observed mass | LiHMDS | lithium |
| AC₅₀ | half-maximal activity | | bis(trimethylsilyl)amide |
| | concentration | MeOH | Methanol |
| ACN | acetonitrile | MeCN | acetonitrile |
| app | apparent (NMR) | m | multiplet (NMR) |
| BH₃•THF | borane-tetrahydrofuran | mg | milligrams |
| | complex | min | minutes |
| BBr₃ | boron tribromide | mL | milliliter |
| Calc'd | calculated | mmol | millimole |
| Cbz-Cl | benzyl chloroformate | mM | millimolar |
| CO₂ | carbon dioxide | M | molarity or molar |
| Cs₂CO₃ | cesium carbonate | MS | mass spectrometry |
| d | deuterated (NMR | MsCl | methanesulfonyl chloride |
| | solvents) | MTBE | methyl tert-butyl ether |
| d | doublet (NMR) | n/a | not applicable |
| dd | doublet of doublets | NBS | N-bromosuccinimide |
| | (NMR) | NH₄ | ammonium |
| DCM | dichloromethane | NH₄OH | ammonium hydroxide |
| DIAD | diisopropyl | NH₄HCO₃ | ammonium bicarbonate |
| | azodicarboxylate | Na₂SO₄ | sodium sulfate |
| DMF | N,N-dimethylformamide | NaBH₃CN | sodium |
| EC₅₀ | half-maximal effective | | cyanoborohydride |
| | concentration | NMI | N-methylimidazole |
| EDCI | 1-Ethyl-3-(3- | NMM | N-methylmorpholine |
| | dimethylaminopropyl)car- | NMR | nuclear magnetic |
| | bodiimide | | resonance |
| ESI | electrospray ionization | NaOH | sodium hydroxide |
| EtOAc | ethyl acetate | PCy₃ | Tricyclohexylphosphine |
| EtOH | ethanol | PdCl₂(dppf) | [1,1′- |
| eq | equivalents | | Bis(diphenylphosphino)fer- |
| g | grams | | rocene]dichloropalladium(II) |
| h | hours | pH | potential of hydrogen |
| H | hydrogen | PPh₃ | triphenyl phosphine |
| HCl | hydrochloric acid | s | singlet (NMR) |
| HPLC | high-performance liquid | SFC | super fluid |
| | chromatography | | chromatography |
| IC₅₀ | half-maximal inhibitory | t | triplet (NMR) |
| | concentration | T3P | Propanephosphonic acid |
| In vacuo | in a vacuum | | anhydride |
| IUPAC | International Union of | TBAB | tetrabutylammonium |
| | Pure and Applied | | bromide |
| | Chemistry | TEA | triethylamine |
| MHz | megahertz | TFA | trifluoroacetic acid |
| J | J-coupling value (NMR) | THF | tetrahydrofuran |
| TFCH | N,N,N′,N′- | TMSCl | trimethylsilyl chloride |
| | tetramethylchloroformamidinium | wt. % | weight percent |
| | hexafluorophosphate | | |

### Intermediate A-1: Synthesis of (2S,4R)—N—((S)-(5-cyclopropyl-6-fluoropyridin-2-yl)(phenyl)methyl)-4-fluoropyrrolidine-2-carboxamide

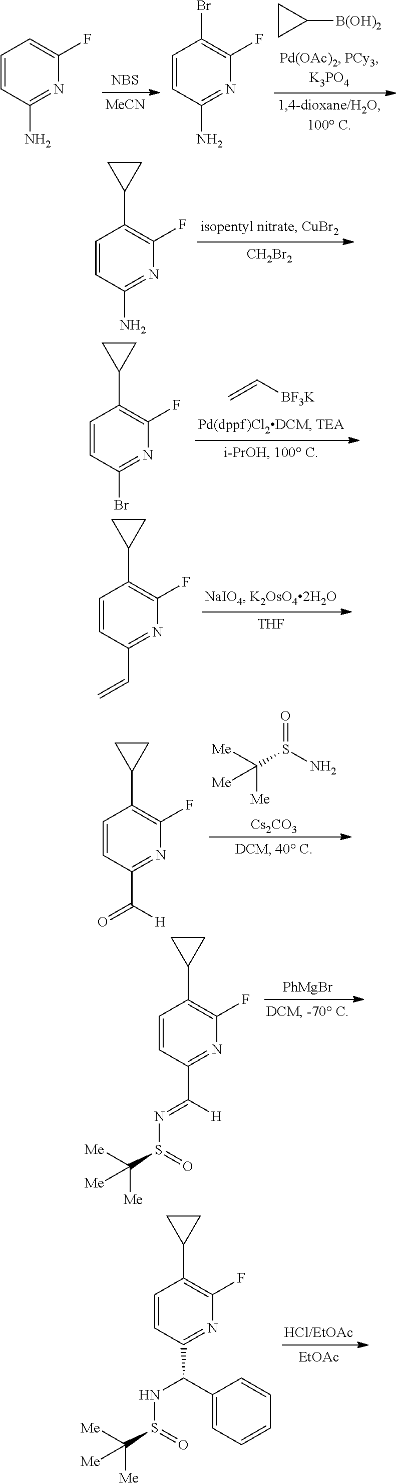

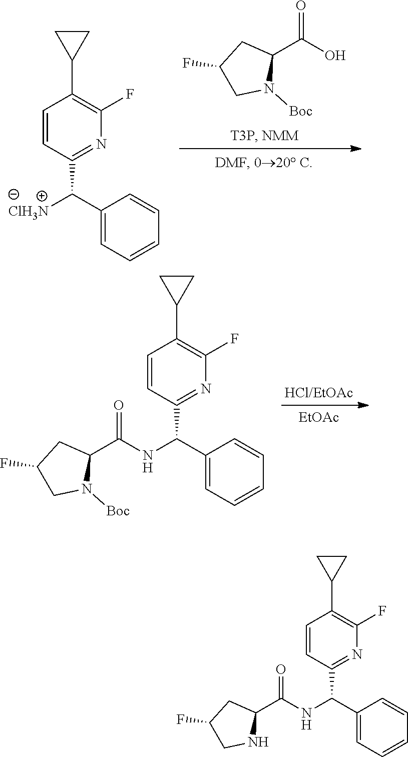

Step a: In four parallel reactions, 6-fluoropyridin-2-amine (125 g, 1.11 mol, 1 eq) in MeCN (1.2 L) at 0° C. under N₂ was treated with NBS (209 g, 1.17 mmol, 1.05 eq) in MeCN (1.2 L). The reaction mixtures were stirred at 20° C. for 2 h. The four parallel reactions were combined, and the resulting mixture was concentrated under reduced pressure. The resulting crude residue was purified by column chromatography to give 5-bromo-6-fluoropyridin-2-amine. LC-MS (ESI): m/z: [M+H]⁺ calculated for C₅H₄BrFN₂: 190.9; found 191.0.

Step b: To a mixture of 5-bromo-6-fluoropyridin-2-amine (200 g, 1.04 mol, 1 eq) and cyclopropylboronic acid (226 g, 2.63 mol, 2.5 eq) in 1,4-dioxane (2 L) and H₂O (200 mL) under N₂ were added K₃PO₄ (666 g, 3.14 mol, 3 eq), PCy₃ (58.6 g, 209 mmol, 0.2 eq), and Pd(OAc)₂ (11.7 g, 52.3 mmol, 0.05 eq). The system was then degassed and charged with nitrogen three times. The reaction mixture was warmed to 100° C. and stirred for 12 h. The reaction mixture was then cooled to room temperature and filtered through Celite. The resulting filtrate was diluted with H₂O (2 L) and then extracted with EtOAc (3×500 mL). The combined organic extracts were washed with brine (2×300 mL), dried over anhydrous Na₂SO₄, filtered, and concentrated under reduced pressure. The crude residue obtained was purified by column chromatography to give 5-cyclopropyl-6-fluoropyridin-2-amine. LC-MS (ESI): m/z: [M+H]⁺ calculated for C₈H₉FN₂: 153.1; found 153.0.

Step c: To a mixture of 5-cyclopropyl-6-fluoropyridin-2-amine (120 g, 788 mmol, 1 eq) in dibromomethane (564 mL) under N₂ was added isopentyl nitrite (110 g, 946 mmol, 127 mL, 1.2 eq). To the resulting mixture was added CuBr₂ (211 g, 946 mmol, 44.3 mL, 1.2 eq) over 0.5 h. The final mixture was then degassed and charged with nitrogen three times before stirring at 20° C. for 16 h. The reaction mixture was then filtered, and the filtrate was diluted with H₂O (500 mL) and extracted with EtOAc (3×300 mL). The combined organic extracts were washed with brine (300 mL), dried over anhydrous Na₂SO₄, filtered, and concentrated under reduced pressure. The crude residue obtained was purified by column chromatography to give 6-bromo-3-cyclopropyl-2-fluoropyridine. LC-MS (ESI): m/z: [M+H]⁺ calculated for C₈H₇BrFN: 216.0; found 216.1.

Step d: To a mixture of 6-bromo-3-cyclopropyl-2-fluoropyridine (90 g, 416 mmol, 1 eq) and trifluoro(vinyl)-λ4-borane, potassium salt (83.7 g, 624 mmol, 1.5 eq) in i-PrOH (900 mL) at 20° C. under N₂ was added TEA (126 g, 1.25 mol, 3 eq) and Pd(dppf)Cl₂·DCM (17 g, 20.8 mmol, 0.05 eq). The resulting mixture was degassed and charged with nitrogen three times. The reaction mixture was then warmed to 100° C. and stirred for 2 h. The reaction mixture was then cooled to room temperature and filtered. The filtrate was diluted with H₂O (500 mL) and extracted with EtOAc (3×300 mL). The combined organic extracts were washed with brine (300 mL), dried over anhydrous Na₂SO₄, filtered, and concentrated under reduced pressure. The crude residue obtained was then purified by column chromatography to give 3-cyclopropyl-2-fluoro-6-vinylpyridine. LC-MS (ESI): m/z: [M+H]⁺ calculated for C₁₀H₁₀FN: 164.1; found 164.1.

Step e: To a mixture of 3-cyclopropyl-2-fluoro-6-vinylpyridine (47 g, 288 mmol, 1 eq) in THF (800 mL) and H₂O (160 mL) at 20° C. under N₂ was added NaIO₄ (246 g, 1.15 mol, 4 eq) and K₂OsO₄·2H₂O (2.12 g, 5.76 mmol, 0.02 eq). The resulting mixture was degassed and charged with nitrogen three times before stirring for 2 h. The reaction mixture was then filtered, and the filtrate was diluted with H₂O (500 mL), and extracted with EtOAc (3×300 mL). The combined organic extracts were washed with brine (300 mL), dried over anhydrous Na₂SO₄, filtered, and concentrated under reduced pressure. The resulting crude residue was purified by column chromatography to give 5-cyclopropyl-6-fluoropicolinaldehyde. LC-MS (ESI): m/z: [M+H]⁺ calculated for C₉H₈FNO: 166.1; found 166.2.

Step f: To a mixture of 5-cyclopropyl-6-fluoropicolinaldehyde (38 g, 230 mmol, 1 eq) and (S)-2-methylpropane-2-sulfinamide (30.6 g, 253 mmol, 1.1 eq) in DCM (200 mL) at 20° C. under N₂ was added Cs₂CO₃ (82.4 g, 253 mmol, 1.1 eq). The system was then degassed and charged with nitrogen three times. The resulting mixture was then warmed to 40° C. and stirred for 12 h. The reaction solution was then diluted with H₂O (300 mL) and extracted with DCM (3×200 mL). The combined organic extracts were washed with brine (200 mL), dried over anhydrous Na₂SO₄, filtered, and concentrated under reduced pressure. The resulting crude residue was then purified by column chromatography to give (S,E)-N-((5-cyclopropyl-6-fluoropyridin-2-yl)methylene)-2-methylpropane-2-sulfinamide. LC-MS (ESI): m/z: [M+H]⁺ calculated for C₁₃H₁₇FN₂OS: 269.1; found 269.2.

Step g: To a solution of (S,E)-N-((5-cyclopropyl-6-fluoropyridin-2-yl)methylene)-2-methylpropane-2-sulfinamide (58 g, 216 mmol, 1 eq) in dry DCM (600 mL) at −70° C. under nitrogen was added PhMgBr (3 M in Et₂O, 93.6 mL, 281 mmol, 1.3 eq) in a dropwise manner. The resulting reaction mixture was stirred at −70° C. for 1 h. The reaction mixture was then quenched with saturated aqueous NH₄Cl solution (500 mL), warmed to room temperature, and extracted with EtOAc (3×200 mL). The combined organic extracts were washed with brine (200 mL), dried over anhydrous Na₂SO₄, filtered, and concentrated under reduced pressure. The crude residue obtained was purified by column chromatography to give (S)—N—((S)-(5-cyclopropyl-6-fluoropyridin-2-yl)(phenyl)methyl)-2-methylpropane-2-sulfinamide. LC-MS (ESI): m/z: [M+H]⁺ calculated for C₁₉H₂₃FN₂OS: 347.2; found 347.3.

Step h: To a solution of (S)—N—((S)-(5-cyclopropyl-6-fluoropyridin-2-yl)(phenyl)methyl)-2-methylpropane-2-sulfinamide (74 g, 213 mmol, 1 eq) in EtOAc (100 mL) at 0° C. under N₂ was added HCl/EtOAc (4 M, 740 mL, 2940 mmol, 13.8 eq). The resulting mixture was then warmed 20° C. and stirred for 1 h. The reaction mixture was then concentrated under reduced pressure, and the crude residue obtained was triturated with MTBE (500 mL). The resulting solid was collected by filtration and dried under reduced pressure to give (S)-(5-cyclopropyl-6-fluoropyridin-2-yl)(phenyl)methanaminium chloride. LC-MS (ESI): m/z: [M+H]⁺ calculated for C₁₅H₁₅FN₂: 243.1; found 243.2.

Step i: To a mixture of (S)-(5-cyclopropyl-6-fluoropyridin-2-yl)(phenyl)methanaminium chloride (56 g, 200 mmol, 1 eq) and (2S,4R)-1-(tert-butoxycarbonyl)-4-fluoropyrrolidine-2-carboxylic acid (60.9 g, 261 mmol, 1.3 eq) in DMF (500 mL) at 0° C. under N₂ was added N-methylmorpholine (NMM, 101 g, 1.00 mol, 110 mL, 5 eq) and T3P (50% in ethyl acetate, 166 g, 261 mmol, 155 mL, 1.3 eq). The resulting mixture was degassed and charged with nitrogen three times, warmed to 20° C. and stirred 1 h. The reaction mixture was then diluted with H₂O (200 mL) and extracted with EtOAc (3×300 mL). The combined organic extracts were washed with brine (500 mL), dried over anhydrous Na₂SO₄, filtered, and concentrated under reduced pressure. The crude residue obtained was purified by column chromatography to give tert-butyl (2S,4R)-2-(((S)-(5-cyclopropyl-6-fluoropyridin-2-yl)(phenyl)methyl)carbamoyl)-4-fluoropyrrolidine-1-carboxylate. LC-MS (ESI): m/z: [M+H]⁺ calculated for C₂₅H₂₉F₂N₃O₃: 458.2; found 458.2.

Step j: To a solution of tert-butyl (2S,4R)-2-(((S)-(5-cyclopropyl-6-fluoropyridin-2-yl)(phenyl)methyl)carbamoyl)-4-fluoropyrrolidine-1-carboxylate (90 g, 196 mmol, 1 eq) in EtOAc (100 mL) at 0° C. under N₂ was added HCl/EtOAc (4 M, 900 mL, 18.3 eq). The resulting mixture was warmed to 20° C. and stirred for 1 h. The reaction mixture was then concentrated under reduced pressure, and the resulting crude residue was added to H₂O (100 mL). The resulting mixture was cooled to 0° C., adjusted to pH=7-8 with saturated aqueous NaHCO₃ and extracted with EtOAc (3×200 mL). The combined organic extracts were washed with brine (100 mL), dried with anhydrous Na₂SO₄, filtered, and concentrated under reduced pressure. The resulting crude material was then triturated with i-PrOH (500 mL), and the resulting solid was isolated via filtration. The solid obtained was dried under reduced pressure to give (2S,4R)—N—((S)-(5-cyclopropyl-6-fluoropyridin-2-yl)(phenyl)methyl)-4-fluoropyrrolidine-2-carboxamide. LC-MS (ESI): m/z: [M+H]⁺ calculated for C₂₀H₂₁F₂N₃O: 358.2; found 358.3.

### Intermediate A-2: Synthesis of 2,2′-((ethane-1,2-diylbis(oxy))bis(1H-1,2,3-triazole-4,1-diyl))diacetic acid

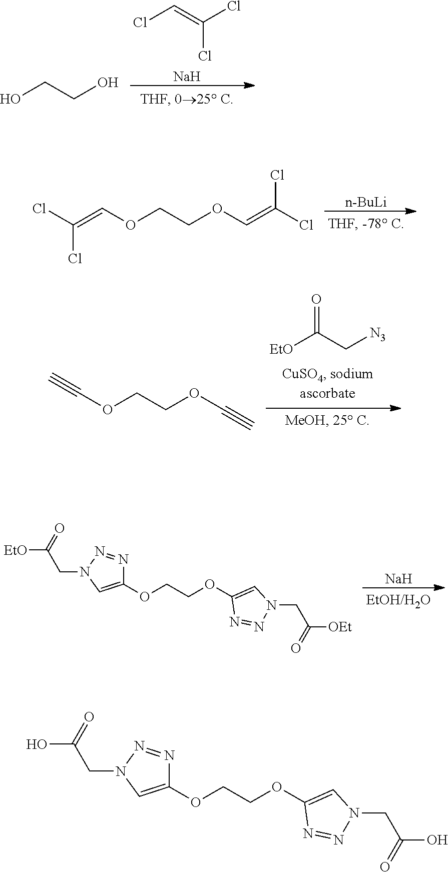

Step a: To a mixture of NaH (3.22 g, 80.5 mmol, 60% in mineral oil, 5 eq) in THF (20 L) at 0° C. under N₂ atmosphere was added ethane-1,2-diol (1.0 g, 16.1 mmol, 1 eq) in a dropwise manner. The resulting mixture was warmed to 25° C. and stirred for 1 h. The reaction mixture was then cooled to 0° C., and 1,1,2-trichloroethene (5.29 g, 40.2 mmol, 2.5 eq) was added in a dropwise manner. The resulting mixture was warmed to 25° C. and stirred for 12 h. The reaction mixture was then cooled to 0° C. and quenched by addition of H₂O (50 mL). The resulting mixture was adjusted to pH 6-7 with aqueous HCl (1N), and the resulting biphasic mixture was extracted with ethyl acetate (2×30 mL). The combined organic extracts were washed with brine (2×40 mL), dried over anhydrous Na₂SO₄, filtered, and concentrated under reduced pressure. The resulting crude residue was purified by column chromatography to give 1,2-bis((2,2-dichlorovinyl)oxy)ethane, which was carried forward to the next step without further characterization.

Step b: To a solution of 1,2-bis((2,2-dichlorovinyl)oxy)ethane (1.0 g, 3.97 mmol, 1 eq) in THF (15 mL) at −78° C. under N₂ atmosphere was added n-BuLi (2.5 M in hexane, 7.94 mL, 5 eq) in a dropwise manner. The resulting mixture was stirred at −78° C. for 1 h. The reaction mixture was then quenched by addition MeOH (10 mL) at −78° C. The reaction mixture was then allowed to warm to 23° C. before it was diluted with H₂O (30 mL). The resulting biphasic mixture was extracted with DCM (3×30 mL). The combined organic extracts were washed with brine (2×50 mL), dried over anhydrous Na₂SO₄, filtered, and concentrated under reduced pressure to give crude 1,2-bis(ethynyloxy)ethane, which was carried forward to the next step without further purification or characterization.

Step c: A mixture of 1,2-bis(ethynyloxy)ethane (0.8 g, 7.27 mmol, 1 eq), ethyl 2-azidoacetate (1.88 g, 14.5 mmol, 2 eq), CuSO₄ (231 mg, 1.45 mmol, 0.2 eq), sodium L-ascorbate (287 mg, 1.45 mmol, 0.2 eq) in MeOH (5 mL) was stirred at 25° C. for 16 h. The reaction mixture was then quenched by addition H₂O (20 mL). The resulting biphasic mixture was then extracted with DCM (2×10 mL). The combined organic extracts were washed with brine (2×20 mL), dried over anhydrous Na₂SO₄, filtered, and concentrated under reduced pressure. The crude residue obtained was purified by prep-HPLC to give diethyl 2,2′-((ethane-1,2-diylbis(oxy))bis(1H-1,2,3-triazole-4,1-diyl))diacetate. LC-MS (ESI): m/z: [M+H]⁺ calculated for C₁₄H₂₀N₆O₆: 369.1; found 369.0.

Step d: To a solution of diethyl 2,2′-((ethane-1,2-diylbis(oxy))bis(1H-1,2,3-triazole-4,1-diyl))diacetate (150 mg, 325 μmol, 1 eq) in THF (2 mL) and H₂O (0.5 mL) was added LiOH·H₂O (68.3 mg, 1.63 mmol, 5 eq). The resulting mixture was stirred at 25° C. for 2 h before it was partially concentrated under reduced pressure. The resulting aqueous mixture was adjusted pH to 5-6 with aqueous HCl (1N). The resulting mixture was concentrated under reduced pressure. The crude residue obtained was purified by prep-HPLC to give 2,2′-((ethane-1,2-diylbis(oxy))bis(1H-1,2,3-triazole-4,1-diyl))diacetic acid. LC-MS (ESI): m/z: [M+H]⁺ calculated for C₁₀H₁₂N₆O₆: 313.1; found 313.1.

The following compounds in Table B-1 were synthesized using procedures similar to Intermediate A-2 using the appropriate starting materials.

TABLE B-1

| | | | Exact | |
|---|---|---|---|---|
| Intermediate | | | mass | |
| No. | Structure | IUPAC | (g/mol) | LCMS, Found [M + H]⁺ |
| B-1-1 | 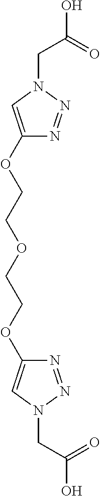 [SMILES: O=C(O)Cn1cc(OCCOCCOc2cn(CC(=O)O)nn2)nn1] | 2,2′- (((oxybis(ethane- 2,1- diyl))bis(oxy))bis(1H- 1,2,3-triazole- 4,1-diyl))diacetic acid | 356.1 | 357.0 |

### Intermediate A-3: Synthesis of 2-(4-(2-((1-(carboxymethyl)-1H-1,2,3-triazol-4-yl)methoxy)ethoxy)-1H-1,2,3-triazol-1-yl)acetic acid

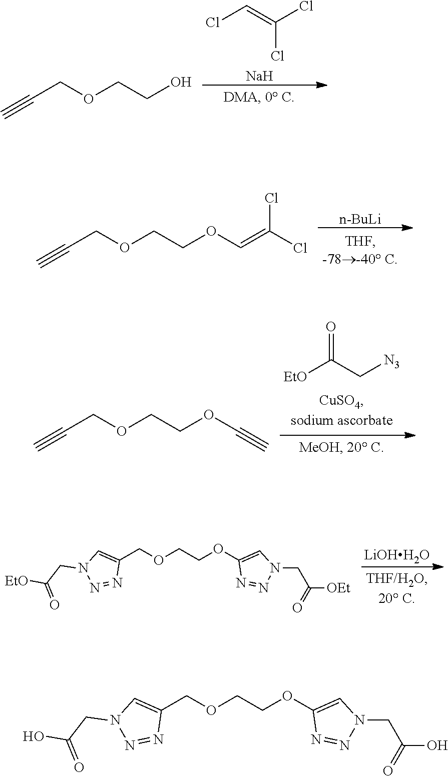

Step a: To a solution of 2-(prop-2-yn-1-yloxy)ethan-1-ol (500 mg, 5.81 mmol, 1 eq) in DMA (4 mL) at 0° C. under N₂ was added NaH (580 mg, 14.5 mmol, 60% in mineral oil, 2.5 eq). The resulting mixture was stirred for 1 h before a solution of 1,1,2-trichloroethene (915 mg, 6.97 mmol, 1.2 eq) in DMA (2 mL) was added at 0° C. The resulting mixture was stirred at 0° C. for 1 h. The reaction mixture was then quenched with saturated aqueous NH₄Cl (20 mL) and extracted with EtOAc (3×10 mL). The combined organic extracts were washed with brine (20 mL), dried over anhydrous Na₂SO₄, filtered, and concentrated under reduced pressure. The crude residue obtained was purified by column chromatography to give 3-(2-((2,2-dichlorovinyl)oxy)ethoxy)prop-1-yne, which was carried forward to the next step without further purification or characterization.

Step b: To a solution of 3-(2-((2,2-dichlorovinyl)oxy)ethoxy)prop-1-yne (600 mg, 3.08 mmol, 1 eq) in THF (5 mL) at −78° C. under N₂ atmosphere was dropwise added n-BuLi (2.5 M in hexane, 3.08 mL, 2.5 eq). The resulting mixture was warmed to −40° C. and stirred for 2 h. The reaction mixture was then quenched with MeOH (2 mL), warmed to 25° C., and stirred for 30 min. The mixture was then extracted with DCM (3×10 mL). The combined organic extracts were washed with brine (20 mL), dried over anhydrous Na₂SO₄, filtered, and concentrated under reduced pressure to give 3-(2-(ethynyloxy)ethoxy)prop-1-yne, which was carried forward to the next step without further purification or characterization.

Step c: To a solution of 3-(2-(ethynyloxy)ethoxy)prop-1-yne (300 mg, 2.42 mmol, 1 eq) and ethyl 2-azidoacetate (1.25 g, 9.67 mmol, 4 eq) in MeOH (2 mL) was added sodium L-ascorbate (95.7 mg, 483 μmol, 0.2 eq) and CuSO₄ (77.1 mg, 483 μmol, 0.2 eq). The resulting mixture was stirred at 25° C. for 16 h. The reaction mixture was then diluted with H₂O (10 mL) and extracted with EtOAc (3×10 mL). The combined organic extracts were washed with brine (20 mL), dried over anhydrous Na₂SO₄, filtered, and concentrated under reduced pressure. The crude residue obtained was purified by column chromatography to give ethyl 2-(4-(2-((1-(2-ethoxy-2-oxoethyl)-1H-1,2,3-triazol-4-yl)methoxy)ethoxy)-1H-1,2,3-triazol-1-yl)acetate. LC-MS (ESI): m/z: [M+H]⁺ calculated for C₁₅H₂₂N₆O₆: 383.2; found 383.1.

Step d: To a solution of ethyl 2-(4-(2-((1-(2-ethoxy-2-oxoethyl)-1H-1,2,3-triazol-4-yl)methoxy)ethoxy)-1H-1,2,3-triazol-1-yl)acetate (140 mg, 366 μmol, 1 eq) in THF (2 mL) and H₂O (0.5 mL) was added LiOH—H₂O (46.0 mg, 1.10 mmol, 3 eq). The resulting mixture was stirred at 20° C. for 3 h. The reaction mixture was then concentrated under reduced pressure to remove THF, the remaining aqueous phase was adjusted to pH 5-6 with aqueous HCl (1 N). The resulting solution was then concentrated under reduced pressure and purified by prep-HPLC to give 2-(4-(2-((1-(carboxymethyl)-1H-1,2,3-triazol-4-yl)methoxy)ethoxy)-1H-1,2,3-triazol-1-yl)acetic acid. LC-MS (ESI): m/z: [M+H]⁺ calculated for C₁₁H₁₄N₆O₆: 327.1; found 327.0.

### Intermediate A-4: Synthesis of 2,2′-(((ethane-1,2-diylbis(oxy))bis(methylene))bis(1H-1,2,3-triazole-4,1-diyl))diacetic acid

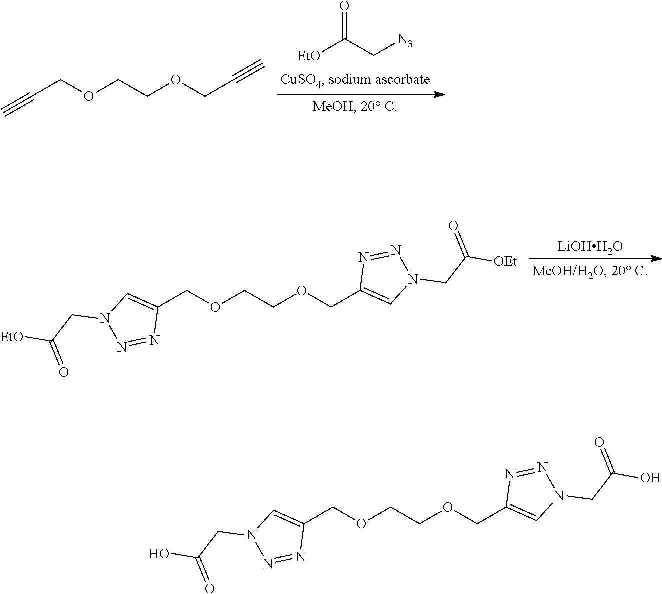

Step a: A mixture of 1,2-bis(prop-2-yn-1-yloxy)ethane (500 mg, 3.62 mmol, 1 eq), ethyl 2-azidoacetate (1.64 g, 12.6 mmol, 1.45 mL, 3.5 eq), CuSO₄ (231 mg, 1.45 mmol, 0.4 eq) and sodium L-ascorbate (286 mg, 1.45 mmol, 0.4 eq) in MeOH (10 mL) was stirred at 20° C. under N₂ atmosphere for 16 h. The reaction mixture was then diluted with H₂O (20 mL) and extracted with EtOAc (3×20 mL). The combined organic extracts were dried over anhydrous Na₂SO₄, filtered, and concentrated under reduced pressure. The crude residue obtained was purified by column chromatography to give diethyl 2,2′-(((ethane-1,2-diylbis(oxy))bis(methylene))bis(1H-1,2,3-triazole-4,1-diyl))diacetate. LC-MS (ESI): m/z: [M+H]⁺ calculated for C₁₆H₂₄N₆O₆: 397.2; found 397.2.

Step b: To a solution of diethyl 2,2′-(((ethane-1,2-diylbis(oxy))bis(methylene))bis(1H-1,2,3-triazole-4,1-diyl))diacetate (200 mg, 504 μmol, 1 eq) in MeOH (3 mL) and H₂O (1 mL) was added LiOH—H₂O (63.5 mg, 1.51 mmol, 3 eq). The resulting mixture was stirred at 20° C. for 2 h. The reaction mixture was then concentrated under reduced pressure to remove MeOH, and the remaining aqueous phase was adjusted to pH 5-6 with aqueous HCl (1 N). The aqueous mixture was then concentrated under reduced pressure. The crude residue obtained was purified by prep-HPLC to give 2,2′-(((ethane-1,2-diylbis(oxy))bis(methylene))bis(1H-1,2,3-triazole-4,1-diyl))diacetic acid. LC-MS (ESI): m/z: [M+H]⁺ calculated for C₁₂H₁₆N₆O₆: 341.1; found 341.1.

### Intermediate A-5: Synthesis of 3,6,9,12,15,18,21-heptaoxatricosanedioic acid

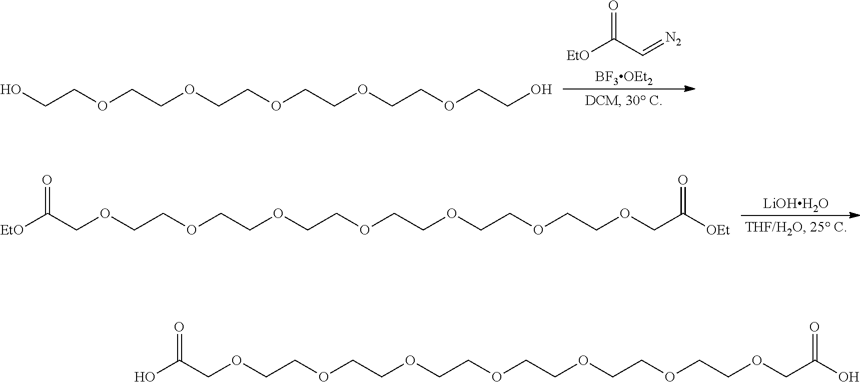

Step a: To a solution of 3,6,9,12,15-pentaoxaheptadecane-1,17-diol (700 mg, 2.48 mmol, 1 eq) and ethyl 2-diazoacetate (1.13 g, 9.92 mmol, 4 eq) in DCM (7 mL) was added BF₃·OEt₂ (35.1 mg, 247 μmol, 0.1 eq). The resulting mixture was stirred at 20° C. for 16 h. The reaction mixture was then diluted with H₂O (20 mL) and extracted with DCM (3×10 mL). The combined organic extracts were washed with brine (20 mL), dried over anhydrous Na₂SO₄, filtered, and concentrated under reduced pressure to give diethyl 3,6,9,12,15,18,21-heptaoxatricosanedioate. LC-MS (ESI): m/z: [M+H]⁺ calculated for C₂₀H₃₈O₁₁: 455.2; found 455.3.

Step b: To a solution of diethyl 3,6,9,12,15,18,21-heptaoxatricosanedioate (700 mg, 1.54 mmol, 1 eq) in THF (8 mL) and H₂O (2 mL) was added LiOH—H₂O (193 mg, 4.62 mmol, 3 eq). The resulting mixture was stirred at 20° C. for 16 h. The reaction mixture was then concentrated under reduced pressure to remove THF, and the remaining aqueous solution was adjusted to pH 5-7 with aqueous HCl (1N). The aqueous mixture was then concentrated under reduced pressure. The crude residue obtained was purified by prep-HPLC to give 3,6,9,12,15,18,21-heptaoxatricosanedioic acid. LC-MS (ESI): m/z: [M/2-H]⁻ calculated for C₁₆H₃₀O₁₁: 198.1; found 198.1.

The following compounds in Table B-2 were synthesized using procedures similar to Intermediate A-5 using the appropriate starting materials.

TABLE B-2

| | | | Exact | |
|---|---|---|---|---|
| Intermediate | | | mass | |
| No. | Structure | IUPAC | (g/mol) | LCMS, Found [M − H]⁻ |
| B-2-1 | 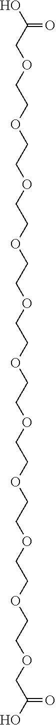 [SMILES: O=C(O)COCCOCCOCCOCCOCCOCCOCCOCCOCCOCCOCC(=O)O] | 3,5,9,12,15,18,21,24, 27,30,33- undecaoxapentatria- contanedioic acid | 574.3 | 573.3 |
| B-2-2 | 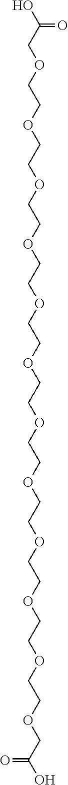 [SMILES: O=C(O)COCCOCCOCCOCCOCCOCCOCCOCCOCCOCCOCCOCC(=O)O] | 3,6,9,12,15,18,21,24, 27,30,33,36- dodecaoxaoctatriacon- tanedioic acid | 618.3 | 617.3 |
| B-2-3 | 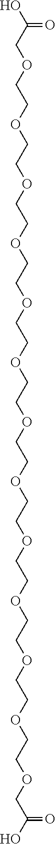 [SMILES: O=C(O)COCCOCCOCCOCCOCCOCCOCCOCCOCCOCCOCCOCCOCC(=O)O] | 3,6,9,12,15,18,21,24, 27,30,33,36,39- tridecaoxahentetracon- tanedioic acid | 662.34 | 680.5 [M + 18]⁺ |

### Intermediate A-6: Synthesis of 2,2′-((oxybis(ethane-2,1-diyl))bis(5-methyl-2,4-dioxo-3,4-dihydropyrimidine-3,1(2H)-diyl))diacetic acid

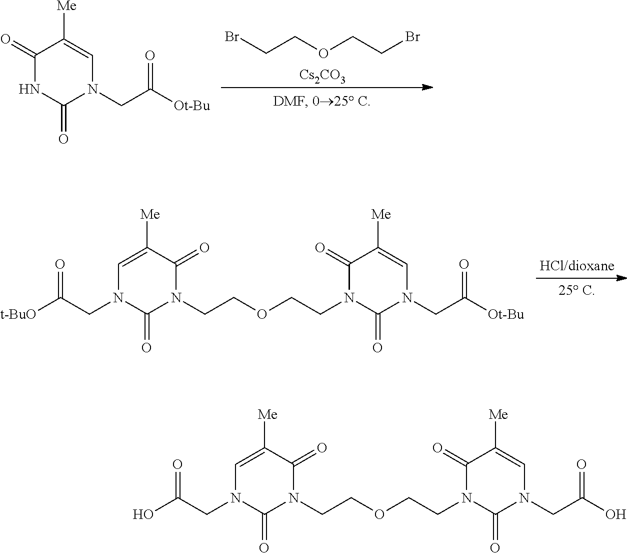

Step a: To a solution of tert-butyl 2-(5-methyl-2,4-dioxo-3,4-dihydropyrimidin-1(2H)-yl)acetate (1 g, 4.16 mmol, 2.2 eq) in DMF (15 mL) at 0° C. was added Cs₂CO₃ (1.85 g, 5.68 mmol, 3 eq) and 1-bromo-2-(2-bromoethoxy)ethane (438 mg, 1.89 mmol, 1 eq), sequentially. The resulting mixture was warmed to 25° C. and stirred for 2 h. The reaction mixture was then quenched by addition water (30 mL), and the resulting biphasic mixture was extracted with EtOAc (3×20 mL). The combined organic extracts were washed with brine (30 mL), dried over anhydrous Na₂SO₄, filtered, and concentrated under reduced pressure. The crude residue obtained was purified by column chromatography to give di-tert-butyl 2,2′-((oxybis(ethane-2,1-diyl))bis(5-methyl-2,4-dioxo-3,4-dihydropyrimidine-3,1(2H)-diyl))diacetate. LC-MS (ESI): m/z: [M+H]⁺ calculated for C₂₆H₃₈N₄O₉: 551.3; found 551.3.

Step b: To a solution of di-tert-butyl 2,2′-((oxybis(ethane-2,1-diyl))bis(5-methyl-2,4-dioxo-3,4-dihydropyrimidine-3,1(2H)-diyl))diacetate (0.5 g, 908.10 μmol, 1 eq) in dioxane (5 mL) was added HCl/dioxane (4 M, 5 mL), and the resulting mixture was stirred at 25° C. for 1 h. The reaction mixture was then concentrated under reduced pressure. The crude residue obtained was triturated with EtOAc (100 mL) at 25° C. for 10 min. The resulting slurry was filtered, and the solid collected was dried under reduced pressure to give 2,2′-((oxybis(ethane-2,1-diyl))bis(5-methyl-2,4-dioxo-3,4-dihydropyrimidine-3,1(2H)-diyl))diacetic acid. LC-MS (ESI): m/z: [M−H]⁻ calculated for C₁₈H₂₂N₄O₉: 437.1; found 437.1.

The following compounds in Table B-3 were synthesized using procedures similar to Intermediate A-6 using the appropriate starting materials.

TABLE B-3

| | | | Exact | |
|---|---|---|---|---|
| Intermediate | | | mass | LCMS, Found |
| No. | Structure | IUPAC | (g/mol) | [M − H]⁻ |
| B-3-1 | 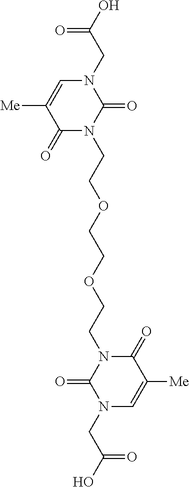 [SMILES: Cc1cn(CC(=O)O)c(=O)n(CCOCCOCCn2c(=O)c(C)cn(CC(=O)O)c2=O)c1=O] | 2,2′-(((ethane-1,2- diylbis(oxy))bis(eth- ane-2,1-diyl))bis(5- methyl-2,4-dioxo- 3,4- dihydropyrimidine- 3,1(2H)- diyl))diacetic acid | 482.2 | 481.1 |

### Intermediate A-7: Synthesis of 2,2′-((ethane-1,2-diylbis(oxy))bis(5-methyl-2-oxopyrimidine-4,1(2H)-diyl))diacetic acid

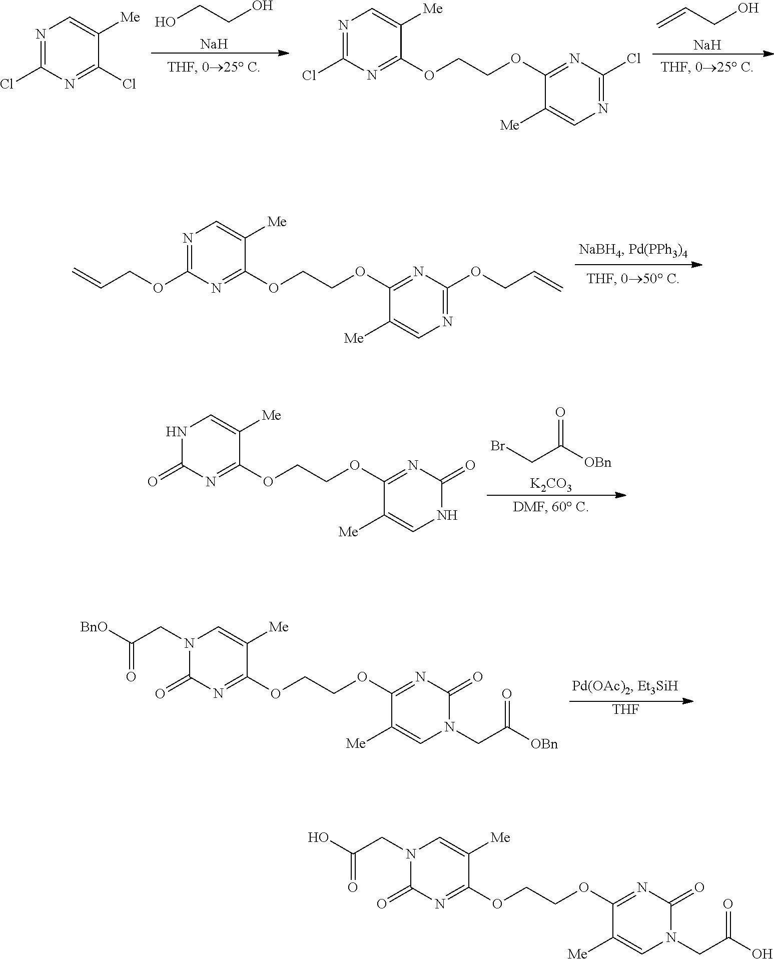

Step a: To a solution of ethylene glycol (1.73 g, 27.89 mmol, 1.56 mL, 1 eq) in THF (100 mL) was at 0° C. under N₂ was added NaH (4.02 g, 83.66 mmol, 50% purity, 3 eq). The resulting mixture was stirred for 1 h before 2,4-dichloro-5-methylpyrimidine (10 g, 61.35 mmol, 2.2 eq) was added at 0° C. The resulting mixture was warmed to 25° C. and stirred for 1 h. The reaction mixture was then cooled to 0° C. and quenched by addition of water (150 mL). The resulting biphasic mixture was extracted with EtOAc (3×100 mL). The combined organic extracts were washed with brine (200 mL), dried over anhydrous Na₂SO₄, filtered, and concentrated under reduced pressure. The crude residue obtained was purified by column chromatography to give 1,2-bis((2-chloro-5-methylpyrimidin-4-yl)oxy)ethane. LC-MS (ESI): m/z: [M+H]⁺ calculated for C₁₂H₁₂Cl₂N₄O₂: 315.0; found 315.3.

Step b: To a solution of NaH (5.14 g, 128.51 mmol, 60% purity, 5 eq) in THF (60 mL) at 0° C. under N₂ was added prop-2-en-1-ol (8.78 g, 151.17 mmol, 5.88 eq). The resulting mixture was stirred at 0° C. for 1 h before a solution of 1,2-bis((2-chloro-5-methylpyrimidin-4-yl)oxy)ethane (8.1 g, 25.70 mmol, 1 eq) in THF (80 mL) was added into the mixture at 0° C. under N₂. The resulting mixture was then warmed to 25° C. and stirred for 4 h under N₂. The reaction mixture was then quenched by addition water (100 mL). The resulting biphasic mixture was extracted with EtOAc (3×100 mL). The combined organic extracts were washed with brine (200 mL), dried over anhydrous Na₂SO₄, filtered, and concentrated under reduced pressure. The crude residue obtained was purified by column chromatography to give 1,2-bis((2-(allyloxy)-5-methylpyrimidin-4-yl)oxy)ethane. LC-MS (ESI): m/z: [M+H]⁺ calculated for C₁₈H₂₂N₄O₄: 359.2; found 359.2.

Step c: To a solution of 1,2-bis((2-(allyloxy)-5-methylpyrimidin-4-yl)oxy)ethane (3.9 g, 10.88 mmol, 1 eq) in THF (40 mL) at 0° C. under N₂ was added NaBH₄ (1.65 g, 43.53 mmol, 4 eq) and Pd(PPh₃)₄ (628 mg, 544.10 μmol, 0.05 eq), sequentially. The resulting mixture was warmed to 50° C. and stirred for 1 h. The reaction mixture was then cooled to 25° C. and quenched by addition of water (50 mL). The resulting biphasic mixture was extracted with EtOAc (3×50 mL). The combined organic extracts were washed with brine (100 mL), dried over anhydrous Na₂SO₄, filtered, and concentrated under reduced pressure. The crude residue obtained was purified by column chromatography to give 4,4′-(ethane-1,2-diylbis(oxy))bis(5-methylpyrimidin-2(1H)-one). LC-MS (ESI): m/z: [M+H]⁺ calculated for C₁₂H₁₄N₄O₄: 279.1; found 279.1.

Step d: To a solution of 4,4′-(ethane-1,2-diylbis(oxy))bis(5-methylpyrimidin-2(1H)-one) (0.5 g, 1.80 mmol, 1 eq) and benzyl 2-bromoacetate (905 mg, 3.95 mmol, 2.2 eq) in DMF (10 mL) was added K₂CO₃ (745 mg, 5.39 mmol, 3 eq). The resulting mixture was warmed to 60° C. and stirred for 2 h. The reaction mixture was then cooled to room temperature and poured into ice water (60 mL). The resulting precipitate was collected via filtration, and the filter cake was washed with water (5×10 mL). The filter cake was then dried under reduced pressure to give dibenzyl 2,2′-((ethane-1,2-diylbis(oxy))bis(5-methyl-2-oxopyrimidine-4,1(2H)-diyl))diacetate. LC-MS (ESI): m/z: [M+H]⁺ calculated for C₃₀H₃₀N₄O₈: 575.2; found 575.3.

Step e: To a solution of dibenzyl 2,2′-((ethane-1,2-diylbis(oxy))bis(5-methyl-2-oxopyrimidine-4,1(2H)-diyl))diacetate (100 mg, 174.04 μmol, 1 eq) in DMF (1 mL) was added Et₃SiH (40 mg, 348.08 μmol, 2 eq), Et₃N (3.5 mg, 34.81 μmol, 0.2 eq), and Pd(OAc)₂ (9.7 mg, 43.51 μmol, 0.25 eq) under N₂. The resulting mixture was warmed to 50° C. and stirred for 1.5 h.

The reaction mixture was then concentrated under reduced pressure. The crude residue obtained was purified by prep-HPLC to give 2,2′-((ethane-1,2-diylbis(oxy))bis(5-methyl-2-oxopyrimidine-4,1(2H)-diyl))diacetic acid. LC-MS (ESI): m/z: [M−H]⁻ calculated for C₁₆H₁₈N₄O: 393.1; found 393.1.

### Intermediate A-8: Synthesis of 3,6,12,15,18-pentaoxa-9-azaicosanedioic acid

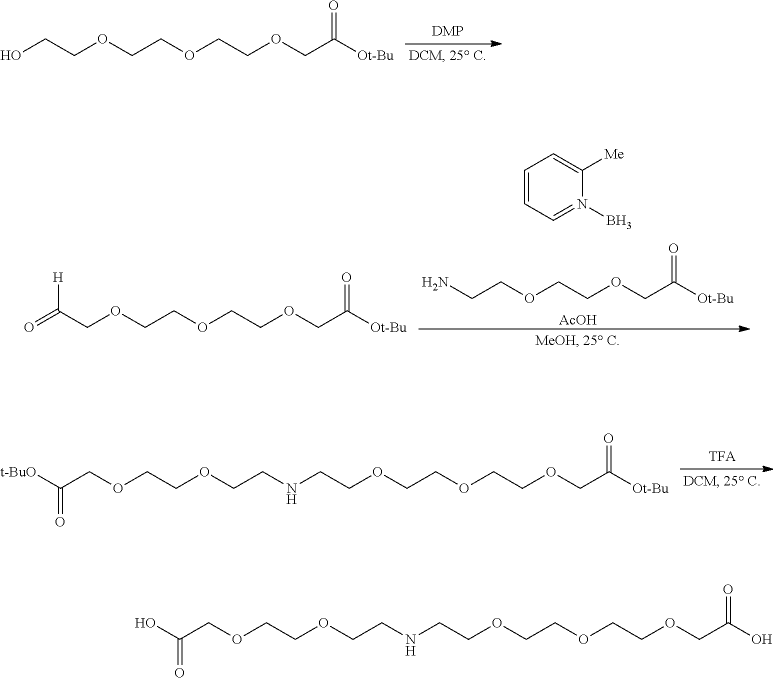

Step a: To a solution of tert-butyl 2-(2-(2-(2-hydroxyethoxy)ethoxy)ethoxy)acetate (1 g, 3.78 mmol, 1 eq) in DCM (60 mL) at 0° C. was added DMP (4.01 g, 9.46 mmol, 2.5 eq). The resulting mixture was warmed to 25° C. and stirred for 3 h. The reaction mixture was then quenched by addition of saturated aqueous Na₂SO₃ (20 mL), and the resulting biphasic mixture was extracted with EtOAc (2×15 mL). The combined organic extracts were washed with brine (20 mL), dried over anhydrous Na₂SO₄, filtered, and concentrated under reduced pressure. The crude residue obtained was purified by column chromatography to give tert-butyl 2-(2-(2-(2-oxoethoxy)ethoxy)ethoxy)acetate, which was carried forward to the next step without further purification or characterization.

Step b: To a solution of tert-butyl 2-(2-(2-(2-oxoethoxy)ethoxy)ethoxy)acetate (95.7 mg, 364.84 μmol, 1.6 eq) and tert-butyl 2-(2-(2-aminoethoxy)ethoxy)acetate (50 mg, 228.02 μmol, 1 eq) in MeOH (2 mL) was added 2-methylpyridine borane complex (73.2 mg, 684.07 μmol, 3 eq) and AcOH (5 mg, 1.75 mmol, 7.67 eq). The resulting mixture was then stirred at 25° C. for 16 h. The reaction mixture was then concentrated under reduced pressure. The crude residue obtained was purified by column chromatography to give di-tert-butyl 3,6,12,15,18-pentaoxa-9-azaicosanedioate, which was carried forward to the next step without further purification or characterization.

Step c: To a solution of di-tert-butyl 3,6,12,15,18-pentaoxa-9-azaicosanedioate (100 mg, 214.79 μmol, 1 eq) in DCM (0.75 mL) was added TFA (770.00 mg, 6.75 mmol, 0.5 mL, 31.44 eq), and the resulting mixture was stirred at 25° C. for 16 h. The reaction mixture was concentrated under reduced pressure to give 3,6,12,15,18-pentaoxa-9-azaicosanedioic acid. LC-MS (ESI): m/z: [M−H]⁻ calculated for C₁₄H₂₇NO₉: 352.2; found 352.1.

### Example S-1: Synthesis of (2S,2′S,4R,4′R)-1,1′-(2,2′-(((oxybis(ethane-2,1-diyl))bis(oxy))bis(1H-1,2,3-triazole-4,1-diyl))bis(acetyl))bis(N—((S)-(5-cyclopropyl-6-fluoropyridin-2-yl)(phenyl)methyl)-4-fluoropyrrolidine-2-carboxamide) (Compound 1)

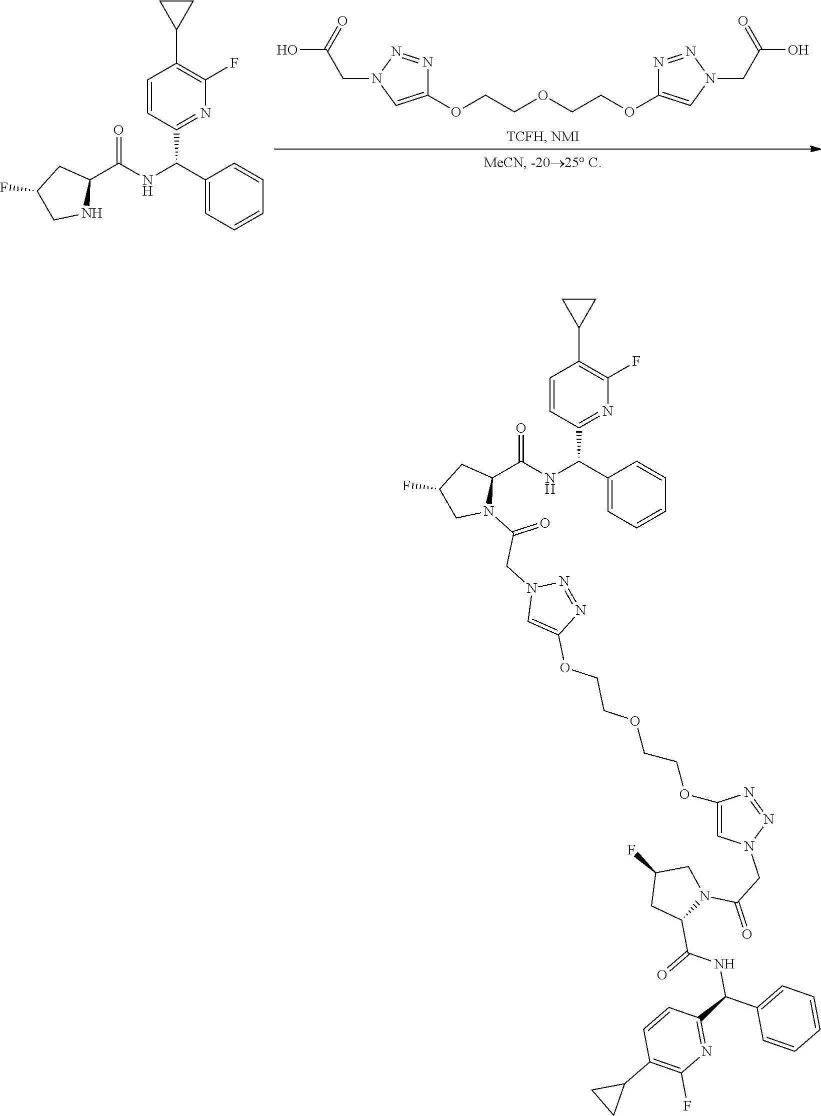 [SMILES: O=C(N[C@@H](c1ccccc1)c1ccc(C2CC2)c(F)n1)[C@@H]1C[C@@H](F)CN1 ; O=C(O)Cn1cc(OCCOCCOc2cn(CC(=O)O)nn2)nn1 ; O=C(N[C@@H](c1ccccc1)c1ccc(C2CC2)c(F)n1)[C@@H]1C[C@@H](F)CN1C(=O)Cn1cc(OCCOCCOc2cn(CC(=O)N3C[C@H](F)C[C@H]3C(=O)N[C@@H](c3ccccc3)c3ccc(C4CC4)c(F)n3)nn2)nn1]

Step a: To a solution of 2,2′-(((oxybis(ethane-2,1-diyl))bis(oxy))bis(1H-1,2,3-triazole-4,1-diyl))diacetic acid (0.1 g, 280 μmol, 1 eq), (2S,4R)—N—((S)-(5-cyclopropyl-6-fluoropyridin-2-yl)(phenyl)methyl)-4-fluoropyrrolidine-2-carboxamide (220 mg, 617 μmol, 2.2 eq), and NMI (230 mg, 2.81 mmol, 10 eq) in CH₃CN (5 mL) at −20° C. under N₂ atmosphere was added TCFH (393 mg, 1.40 mmol, 5 eq). The resulting mixture was warmed to 25° C. and stirred for 3 h. The reaction mixture was then quenched by addition of H₂O (10 mL). The resulting biphasic mixture was then extracted with EtOAc (3×10 mL). The combined organic extracts were washed with brine (20 mL), dried over anhydrous Na₂SO₄, filtered, and concentrated under reduced pressure. The crude residue obtained was purified by prep-HPLC to give (2S,2′S,4R,4′R)-1,1′-(2,2′-(((oxybis(ethane-2,1-diyl))bis(oxy))bis(1H-1,2,3-triazole-4,1-diyl))bis(acetyl))bis(N—((S)-(5-cyclopropyl-6-fluoropyridin-2-yl)(phenyl)methyl)-4-fluoropyrrolidine-2-carboxamide). ¹H NMR (3.2:1 rotamer ratio, asterisk denotes distinct minor rotamer peaks, obscured peaks not reported, 400 MHz, DMSO-d₆) δ 9.28* (d, J=7.8 Hz, 2H), 8.92 (d, J=8.0 Hz, 2H), 7.60-7.46 (m, 4H), 7.42-7.18 (m, 12H), 6.11* (d, J=7.7 Hz, 2H), 5.97 (d, J=8.0 Hz, 2H), 5.54-5.20 (m, 6H), 4.93* (t, J=8.0 Hz, 2H), 4.70-4.59 (m, 2H), 4.26-4.16 (m, 4H), 4.10-3.88 (m, 2H), 3.88-3.66 (m, 6H), 2.87-2.69* (m, 2H), 2.30-2.11* (m, 2H), 2.11-2.01* (m, 2H), 2.01-1.89 (m, 3H), 1.02-0.91 (m, 4H), 0.80-0.68 (in, 4H). LC-MS (ESI): m/z: [M+H]⁺ calculated for C₅₂H₅₄F₄N₁₂O₇: 1035.4; found 1035.4.

The following compounds in Table T-1 were synthesized using procedures similar to Compound 1 using the appropriate starting materials.

TABLE T-1

| | | | Exact | LCMS, |
|---|---|---|---|---|
| Cmpd | | | mass | Found [M + |
| No. | Structure | IUPAC | (g/mol) | H]⁺ |
| 2 | 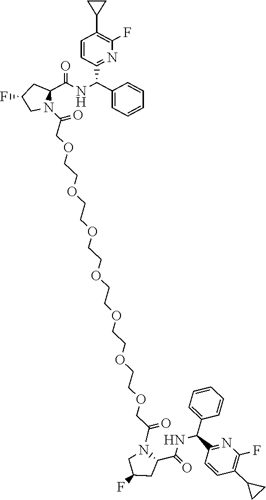 [SMILES: O=C(N[C@@H](c1ccccc1)c1ccc(C2CC2)c(F)n1)[C@@H]1C[C@@H](F)CN1C(=O)COCCOCCOCCOCCOCCOCCOCC(=O)N1C[C@H](F)C[C@H]1C(=O)N[C@@H](c1ccccc1)c1ccc(C2CC2)c(F)n1] | (2S,2′S,4R,4′R)-1,1′- (3,6,9,12,15,18,21- heptaoxatricosanedioyl)bis (N-((S)-(5-cyclopropyl- 6-fluoropyridin-2- yl)(phenyl)methyl)-4- fluoropyrrolidine-2- carboxamide) | 1076.5 | 1077.3 |
| 3 | 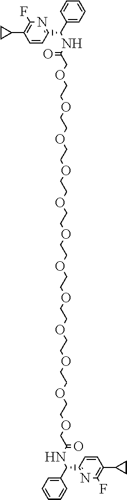 [SMILES: O=C(COCCOCCOCCOCCOCCOCCOCCOCCOCCOCCOCCOCC(=O)N[C@@H](c1ccccc1)c1ccc(C2CC2)c(F)n1)N[C@@H](c1ccccc1)c1ccc(C2CC2)c(F)n1] | N1,N38-bis((S)-(5- cyclopropyl-6- fluoropyridin-2- yl)(phenyl)methyl)- 3,6,9,12,15,18,21,24,27, 30,33,36- dodecaoxactatriacontane- diamide | 1066.5 | 1067.3 |
| 4 | 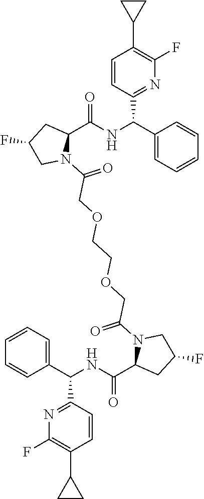 [SMILES: O=C(N[C@@H](c1ccccc1)c1ccc(C2CC2)c(F)n1)[C@@H]1C[C@@H](F)CN1C(=O)COCCOCC(=O)N1C[C@H](F)C[C@H]1C(=O)N[C@@H](c1ccccc1)c1ccc(C2CC2)c(F)n1] | (2S,2′S,4R,4′R)-1,1′-(2,2′- (ethane-1,2- diylbis(oxy))bis(acetyl)) bis(N-((S)-(5- cyclopropyl-6- fluoropyridin-2- yl)(phenyl)methyl)-4- fluoropyrrolidine-2- carboxamido) | 856.4 | 857.1 |
| 5 | 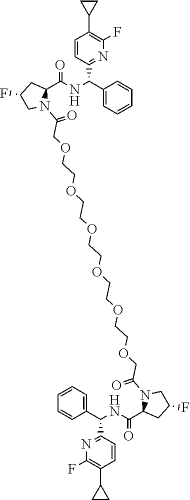 [SMILES: O=C(N[C@@H](c1ccccc1)c1ccc(C2CC2)c(F)n1)[C@@H]1C[C@@H](F)CN1C(=O)COCCOCCOCCOCCOCCOCC(=O)N1C[C@H](F)C[C@H]1C(=O)N[C@@H](c1ccccc1)c1ccc(C2CC2)c(F)n1] | (2S,2′S,4R,4′R)-1,1′- (3,6,9,12,15,18- hexaoxaicosanedioyl)bis(N- ((S)-(5-cyclopropyl-6- fluoropyridin-2- yl)(phenyl)methyl)-4- fluoropyrrolidine-2- carboxamide) | 1032.5 | 1033.6 |
| 6 | 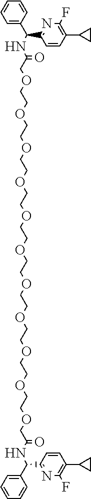 [SMILES: O=C(COCCOCCOCCOCCOCCOCCOCCOCCOCCOCCOCC(=O)N[C@@H](c1ccccc1)c1ccc(C2CC2)c(F)n1)N[C@@H](c1ccccc1)c1ccc(C2CC2)c(F)n1] | N¹,N³⁵-bis((S)-(5- cyclopropyl-6- fluoropyridin-2- yl)(phenyl)methyl)- 3,6,9,12,15,18,21,24,27, 30,33- undecaoxapentatriacontane- diamide | 1022.5 | 1023.3 |
| 7 | 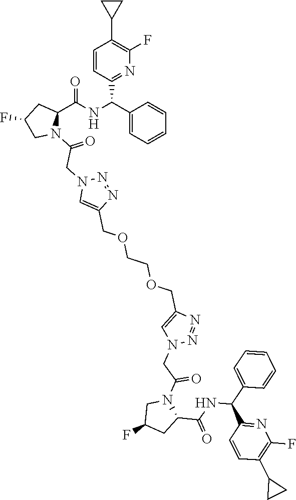 [SMILES: O=C(N[C@@H](c1ccccc1)c1ccc(C2CC2)c(F)n1)[C@@H]1C[C@@H](F)CN1C(=O)Cn1cc(COCCOCc2cn(CC(=O)N3C[C@H](F)C[C@H]3C(=O)N[C@@H](c3ccccc3)c3ccc(C4CC4)c(F)n3)nn2)nn1] | (2S,2′S,4R,4′R)-1,1′-(2,2′- (((ethane-1,2- diylbis(oxy))bis(methylene)) bis(1H-1,2,3- triazole-4,1- diyl))bis(acetyl))bis(N- ((S)-(5-cyclopropyl-6- fluoropyridin-2- yl)(phenyl)methyl)-4- fluoropyrrolidine-2- carboxamide) | 1018.4 | 1019.2 |
| 8 | 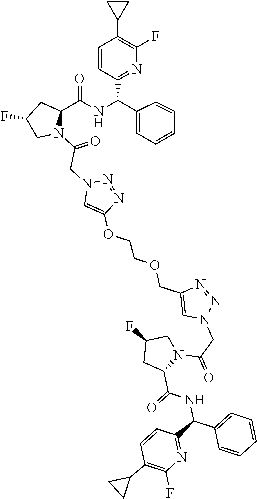 [SMILES: O=C(N[C@@H](c1ccccc1)c1ccc(C2CC2)c(F)n1)[C@@H]1C[C@@H](F)CN1C(=O)Cn1cc(COCCOc2cn(CC(=O)N3C[C@H](F)C[C@H]3C(=O)N[C@@H](c3ccccc3)c3ccc(C4CC4)c(F)n3)nn2)nn1] | (2S,4R)-N-((S)-(5- cyclopropyl-6- fluoropyridin-2- yl)(phenyl)methyl)-1-(2- (4-(2-((1-(2-((2S,4R)-2- (((S)-(5-cyclopropyl-6- fluoropyridin-2- yl)(phenyl)methyl)caba- moyl)-4- fluoropyrrolidin-1-yl)-2- oxoethyl)-1H-1,2,3- triazol-4- yl)methoxy)ethoxy)-1H- 1,2,3-triazol-1-yl)acetyl)- 4-fluoropyrrolidine-2- carboxamide | 1004.4 | 1005.3 |
| 9 | 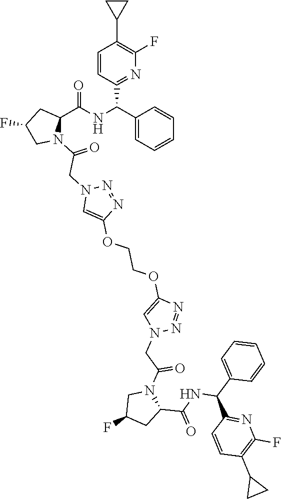 [SMILES: O=C(N[C@@H](c1ccccc1)c1ccc(C2CC2)c(F)n1)[C@@H]1C[C@@H](F)CN1C(=O)Cn1cc(OCCOc2cn(CC(=O)N3C[C@H](F)C[C@H]3C(=O)N[C@@H](c3ccccc3)c3ccc(C4CC4)c(F)n3)nn2)nn1] | (2S,2′S,4R,4′R)-1,1′-(2,2′- ((ethane-1,2- diylbis(oxy))bis(1H- 1,2,3-triazole-4,1- diyl))bis(acetyl))bis(N- ((S)-(5-cyclopropyl-6- fluoropyridin-2- yl)(phenyl)methyl)-4- fluoropyrrolidine-2- carboxamide | 990.4 | 991.4 |
| 10 | 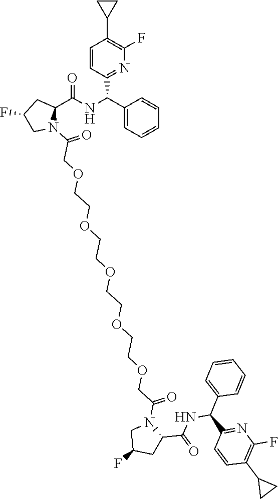 [SMILES: O=C(N[C@@H](c1ccccc1)c1ccc(C2CC2)c(F)n1)[C@@H]1C[C@@H](F)CN1C(=O)COCCOCCOCCOCCOCC(=O)N1C[C@H](F)C[C@H]1C(=O)N[C@@H](c1ccccc1)c1ccc(C2CC2)c(F)n1] | (2S,2′S,4R,4′R)-1,1′- (3,6,9,12,15- pentaoxaheptadecanedioyl) bis(N-((S)-(5- cyclopropyl-6- fluoropyridin-2- yl)(phenyl)methyl)-4- fluoropyrrolidine-2- carboxamide) | 988.4 | 989.4 |
| 11 | 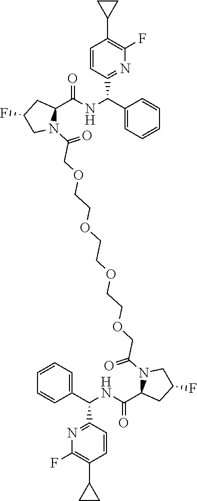 [SMILES: O=C(N[C@@H](c1ccccc1)c1ccc(C2CC2)c(F)n1)[C@@H]1C[C@@H](F)CN1C(=O)COCCOCCOCCOCC(=O)N1C[C@H](F)C[C@H]1C(=O)N[C@@H](c1ccccc1)c1ccc(C2CC2)c(F)n1] | (2S,2′S,4R,4′R)-1,1′- (3,6,9,12- tetraoxatetradecanedioyl) bis(N-((S)-(5- cyclopropyl-6- fluoropyridin-2- yl)(phenyl)methyl)-4- fluoropyrrolidine-2- carboxamide) | 944.4 | 945.1 |
| 12 | 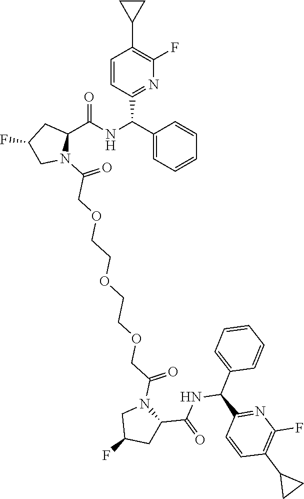 [SMILES: O=C(N[C@@H](c1ccccc1)c1ccc(C2CC2)c(F)n1)[C@@H]1C[C@@H](F)CN1C(=O)COCCOCCOCC(=O)N1C[C@H](F)C[C@H]1C(=O)N[C@@H](c1ccccc1)c1ccc(C2CC2)c(F)n1] | (2S,2′S,4R,4′R)-1,1′-(2,2′- ((oxybis(ethane-2,1- diyl))bis(oxy))bis(acetyl)) bis(N-((S)-(5- cyclopropyl-6- fluoropyridin-2- yl)(phenyl)methyl)-4- fluoropyrrolidine-2- carboxamide | 900.4 | 901.4 |
| 13 | 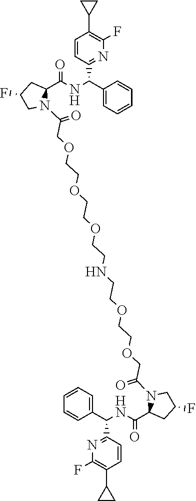 [SMILES: O=C(N[C@@H](c1ccccc1)c1ccc(C2CC2)c(F)n1)[C@@H]1C[C@@H](F)CN1C(=O)COCCOCCNCCOCCOCCOCC(=O)N1C[C@H](F)C[C@H]1C(=O)N[C@@H](c1ccccc1)c1ccc(C2CC2)c(F)n1] | (2S,2′S,4R,4′R)-1,1′- (3,6,12,15,18-pentaoxa- 9-azaicosanedioyl)bis(N- ((S)-(5-cyclopropyl-6- fluoropyridin-2- yl)(phenyl)methyl)-4- fluoropyrrolidine-2- carboxamide | 1031.5 | 1032.4 |
| 14 | 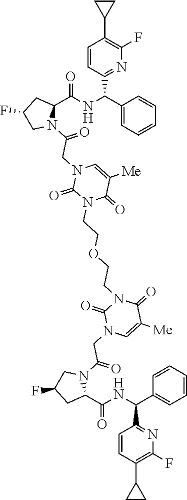 [SMILES: Cc1cn(CC(=O)N2C[C@H](F)C[C@H]2C(=O)N[C@@H](c2ccccc2)c2ccc(C3CC3)c(F)n2)c(=O)n(CCOCCn2c(=O)c(C)cn(CC(=O)N3C[C@H](F)C[C@H]3C(=O)N[C@@H](c3ccccc3)c3ccc(C4CC4)c(F)n3)c2=O)c1=O] | (2S,2′S,4R,4′R)-1,1′-(2,2′- ((oxybis(ethane-2,1- diyl))bis(5-methyl-2,4- dioxo-3,4- dihydropyrimidine- 3,1(2H)- diyl))bis(acetyl))bis(N- ((S)-(5-cyclopropyl-6- fluoropyridin-2- yl)(phenyl)methyl)-4- fluoropyrrolidine-2- carboxamide) | 1116.4 | 1117.4 |
| 15 | 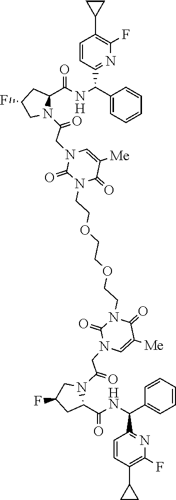 [SMILES: Cc1cn(CC(=O)N2C[C@H](F)C[C@H]2C(=O)N[C@@H](c2ccccc2)c2ccc(C3CC3)c(F)n2)c(=O)n(CCOCCOCCn2c(=O)c(C)cn(CC(=O)N3C[C@H](F)C[C@H]3C(=O)N[C@@H](c3ccccc3)c3ccc(C4CC4)c(F)n3)c2=O)c1=O] | (2S,2′S,4R,4′R)-1,1′-(2,2′- (((ethane-1,2- diylbis(oxy))bis(ethane- 2,1-diyl))bis(5-methyl- 2,4-dioxo-3,4- dihydropyrimidine- 3,1(2H)- diyl))bis(acetyl))bis(N- ((S)-(5-cyclopropyl-6- fluoropyridin-2- yl)(phenyl)methyl)-4- fluoropyrrolidine-2- carboxamide) | 1160.5 | 1161.4 |
| 16 | 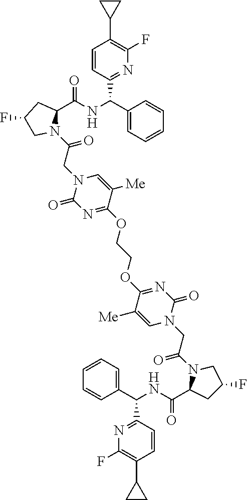 [SMILES: Cc1cn(CC(=O)N2C[C@H](F)C[C@H]2C(=O)N[C@@H](c2ccccc2)c2ccc(C3CC3)c(F)n2)c(=O)nc1OCCOc1nc(=O)n(CC(=O)N2C[C@H](F)C[C@H]2C(=O)N[C@@H](c2ccccc2)c2ccc(C3CC3)c(F)n2)cc1C] | (2S,2′S,4R,4′R)-1,1′-(2,2′- ((ethane-1,2- diylbis(oxy))bis(5- methyl-2-oxopyrimidine- 4,1-(2H)- diyl))bis(acetyl))bis(N- ((S)-(5-cyclopropyl-6- fluoropyridin-2- yl)(phenyl)methyl)-4- fluoropyrrolidine-2- carboxamide) | 1072.4 | 1073.4 |

### Example S-2: Synthesis of (2S,2′S,4R,4′R)-1,1′-(9-methyl-3,6,12,15,18-pentaoxa-9-azaicosanedioyl)bis(N—((S)-(5-cyclopropyl-6-fluoropyridin-2-yl)(phenyl)methyl)-4-fluoropyrrolidine-2-carboxamide) (Compound 17)

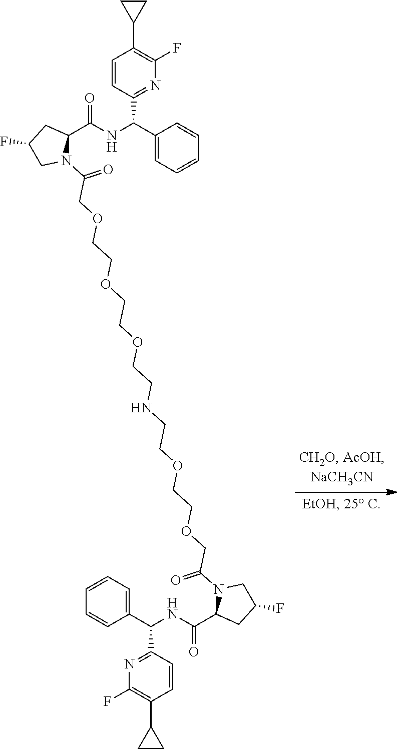 [SMILES: O=C(N[C@@H](c1ccccc1)c1ccc(C2CC2)c(F)n1)[C@@H]1C[C@@H](F)CN1C(=O)COCCOCCNCCOCCOCCOCC(=O)N1C[C@H](F)C[C@H]1C(=O)N[C@@H](c1ccccc1)c1ccc(C2CC2)c(F)n1]

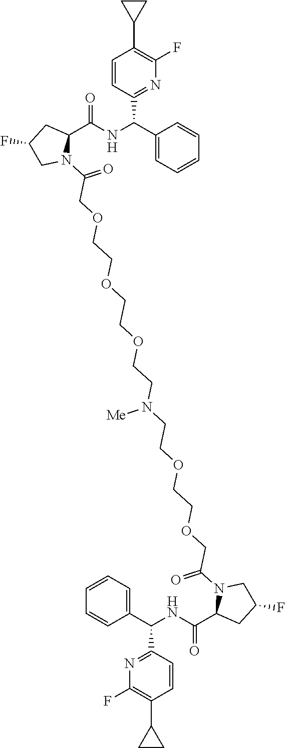 [SMILES: CN(CCOCCOCCOCC(=O)N1C[C@H](F)C[C@H]1C(=O)N[C@@H](c1ccccc1)c1ccc(C2CC2)c(F)n1)CCOCCOCC(=O)N1C[C@H](F)C[C@H]1C(=O)N[C@@H](c1ccccc1)c1ccc(C2CC2)c(F)n1]

Step a: To a solution of (2S,2′S,4R,4′R)-1,1′-(3,6,12,15,18-pentaoxa-9-azaicosanedioyl)bis(N—((S)-(5-cyclopropyl-6-fluoropyridin-2-yl)(phenyl)methyl)-4-fluoropyrrolidine-2-carboxamide) (25 mg, 24.22 μmol, 1 eq) and formaldehyde (1 mL, 37% aq.) in EtOH (1 mL) was added acetic acid (2 mg, 24.22 μmol, 1 eq) and sodium cyanoborohydride (3.1 mg, 48.44 μmol, 2 eq). The resulting mixture was stirred at 25° C. for 12 h. The reaction mixture was then concentrated under reduced pressure. The crude residue obtained was purified by prep-HPLC to give (2S,2′S,4R,4′R)-1,1′-(9-methyl-3,6,12,15,18-pentaoxa-9-azaicosanedioyl)bis(N—((S)-(5-cyclopropyl-6-fluoropyridin-2-yl)(phenyl)methyl)-4-fluoropyrrolidine-2-carboxamide). ¹H NMR (3.1:1 rotamer ratio, asterisk denotes distinct minor rotamer peaks, obscured peaks not reported, 400 MHz, DMSO-d₆)¹H NMR (400 MHz, DMSO) δ 8.90 (d, 1H), 7.54 (t, J=8.9 Hz, 2H), 7.37-7.22 (m, 11H), 6.06* (d, J=8.0 Hz, 1H), 5.99 (d, J=8.1 Hz, 1H), 5.46-5.16 (m, 2H), 4.62 (t, J=8.5 Hz, 1H), 4.17-3.98 (m, 3H), 3.91-3.79 (m, 2H), 3.75-3.37 (m, 20H), 2.18 (s, 3H), 2.08-1.82 (m, 4H), 1.00-0.94 (m, 4H), 0.77-0.72 (m, 4H). LC-MS (ESI): m/z: [M+H]⁺ calculated for C₅₅H₆₇F₄N₇O₉: 1046.5; found 1046.4.

### BIOLOGICAL EXAMPLES

### Example B-1

The GYS1 coupled enzyme assay is a kinetic biochemical assay that indirectly quantifies the rate of glycogen synthesis by coupling the conversion of GYS1 substrate UDP-glucose into UDP with downstream enzymatic reactions. UDP is released from UDP-glucose as glucose monomers are linked into the growing glycogen strand by GYS1. The coupled assay then proceeds with pyruvate kinase utilizing UDP and phospho(enol)pyruvate (PEP) to form pyruvate. Lactate dehydrogenase then converts pyruvate and NADH into lactate and NAD+. Oxidation of NADH to NAD+ can be measured continuously with a plate reader by quantifying the decrease in NADH absorbance at 340 nm over time.

Compounds that inhibit the hGYS1 enzyme and, subsequently, the downstream conversion of NADH to NAD+, were tested using assay ready plates (black, clear bottom 384 well plates) in a final DMSO reaction volume of 2.5% DMSO. The Assay Buffer contained 50 mM Tris pH 7.5, 2 mM MgCl₂, and 100 mM KCl. Fresh stocks of BSA at a final concentration of 0.02% and TCEP at 1 mM were added before splitting buffer into hGYS1 buffer and substrate buffer. To the hGYS1 buffer, rabbit liver glycogen was added at a final concentration of 0.2% glycogen. Glucose-6-Phosphate was added at 1 mM, recombinant hGYS1/GN1 protein was added at 50 nM to the substrate buffer, phosphoenolpyruvate (PEP) was added at 2 mM, UDP-Glucose was added at 0.8 mM, NADH) was added at 0.6 mM, and Pyruvate Kinase/Lactate Dehydrogenase was added at 20 units/mL. The reaction was initiated by mixing hGYS1 buffer and substrate buffer at a 1:1 ratio. Both buffers were plated using a liquid dispensing device with hGYS1 buffer plated first followed by the substrate buffer. Plates were spun briefly to eliminate air bubbles and are immediately read in continuous mode at an absorbance of 340 nm, for 10 time points in one-minute increments, for a total of 10 minutes. The slope from these 10 time points was normalized to the positive and negative control wells. The duplicate % inhibition values are then averaged and fit to a Hill equation for dose response according to the Levenberg-Marquardt algorithm with the Hill equation maximum set to 100 and the minimum set to 0.

The results are shown in Table 2 below, which reports the IC₅₀ of each compound. Unless otherwise specified, IC₅₀ values are reported as the geometric mean of at least 2 assay runs on separate days. Each run represents the average of a technical replicate, where each compound was assayed twice in the same plate. As shown in the table below, the compounds of the present invention are potent inhibitors of human GYS1. A superscript “#” symbol in Table 2 indicates results from a single assay run.

Note that, in Table 2, the compounds are referred to by the corresponding Compound No. in Table 1, which is also referred to in the synthetic examples.

TABLE 2

| Cmpd | PK/LDH |
|---|---|
| No. | IC50 (nM) |
| 1 | 10.8 |
| 2 | 5.6 |
| 3 | 5706.0 |
| 4 | 8911.0 |
| 5 | 5.4 |
| 6 | 7504.0 |
| 7 | 11.2 |
| 8 | 5.2 |
| 9 | 20.1 |
| 10 | 14.4 |
| 11 | 14.6 |
| 12 | 82.1 |
| 13 | >10000.0 |
| 14 | 4.9 |
| 15 | 5.3 |
| 16 | 23.0 |
| 17 | 7.5 |

### Example B-2

The GYS1 cell based assay is a bioluminescent assay that can quantify the glucose resulting from glycogen digestion; the quantified glucose is an indirect measure of GYS1 glycogen synthesis. Newly synthesized glycogen is digested using Glucoamylase; the resulting glucose is quantified by using the Glucose-glo assay kit from Promega. Glucose-glo would work by coupling glucose oxidation and NADH production with a bioluminescent system that is activated with NADH. Glucose is oxidized by Glucose dehydrogenase and the reaction reduces NAD+ to NADH; NADH activates Reductase which reduces a pro-luciferin Reductase Substrate to luciferin. Luciferin is detected in a luciferase reaction using Ultra-Glo rLuciferase and ATP, and the luminesce produced is proportional to the glucose in the sample. The luminescence is measured as a single point read in a plate reader.

Compounds that inhibit the hGYS1 enzyme and, subsequently, the glycogen synthesis in cells, can be tested using assay ready plates (white, clear bottom 384 well plates) in a final DMSO reaction volume of 1% DMSO. Compounds in the assay ready plates are mixed with media with no additives, except for 20 mM glucose prior to cell addition. HeLa cells are starved in media with no additives, except for 1× Glutamax for 24 h. Starved HeLa cells were plated, in a 1:1 ratio to the media in the assay ready plate and incubated for 24 h at 37° C. and 5% CO₂. Cells are washed in 1×PBS buffer and lysed in lysis buffer containing 50% 1×PBS and 25% 0.3 N HCl of the final volume in the well or reaction volume; cells are incubated with lysis buffer for 10 minutes and quenched with the remaining 25% of the reaction volume that consists of 450 mM Tris pH 8.0. Lysates are mixed in a 1:1 ratio with Glucoamylase in 100 mM Sodium Acetate buffer, pH 5.3; the mixture is incubated for 1 h at 37° C. The digested lysate is mixed in a 1:1 ratio with Glucose-glo detection mixture as per vendor recommendations (Luciferase detection buffer, Reductase, Reductase substrate, Glucose dehydrogenase, and NAD) in read-out plates (solid white 384-well plates) and incubated for 1 h at RT. The plates are read using a plate reader with luminescence capabilities. Each compound concentration Relative Luminescence Unit (RLU) can be averaged and normalized to the average RLU of the positive and negative controls to obtain a percentage inhibition. The normalized data vs. concentration can be plotted; to determine the half-maximal concentration (IC₅₀), the Levenberg-Marquardt algorithm would be used to fit a Hill equation to the dose response data.

The results would report the IC₅₀ of each compound. Unless otherwise specified, IC₅₀ values would be reported as the geometric mean of at least 2 assay runs on separate days. The compounds of the present invention would be shown as potent inhibitors of human GYS1. Unless otherwise specified, IC₅₀ values would be reported as the geometric mean of at least two assay runs on separate days. Each run would represent the average of a technical replicate, where each compound was assayed twice in the same plate.

### Example B-3

The GYS2 coupled enzyme assay is a kinetic biochemical assay that can indirectly quantify the rate of glycogen synthesis by coupling the conversion of GYS2 substrate UDP-glucose into UDP with downstream enzymatic reactions. UDP is released from UDP-glucose as glucose monomers are linked into the growing glycogen strand by GYS2. The coupled assay then proceeds with pyruvate kinase utilizing UDP and phospho(enol)pyruvate (PEP) to form pyruvate. Lactate dehydrogenase then converts pyruvate and NADH into lactate and NAD+. Oxidation of NADH to NAD+ is measured continuously with a plate reader by quantifying the decrease in NADH absorbance at 340 nm over time.

Compounds that inhibit the hGYS2 enzyme and, subsequently, the downstream conversion of NADH to NAD+, would be tested using assay ready plates (black, clear bottom 384 well plates) in a final DMSO reaction volume of 2.5% DMSO. The Assay Buffer contained 50 mM Tris pH 7.5, 2 mM MgCl₂, and 100 mM KCl. Fresh stocks of BSA at a final concentration of 0.02% and TCEP 1 mM would be added before splitting buffer into hGYS2 buffer and substrate buffer. To the hGYS2 buffer, rabbit liver glycogen would be added at a final concentration of 0.2% glycogen. Glucose-6-Phosphate would be added at 2 mM, recombinant hGYS2/GN1 protein would be added at 200 nM to the substrate buffer, phosphoenolpyruvate (PEP) would be added at 2 mM, UDP-Glucose would be added at 2 mM, NADH would be added at 0.6 mM, and Pyruvate Kinase/Lactate Dehydrogenase would be added at 20 units/mL. The reaction would be initiated by mixing hGYS2 buffer and substrate buffer at a 1:1 ratio. Both buffers would be plated using a liquid dispensing device with hGYS2 buffer plated first followed by the substrate buffer. Plates would be spun briefly to eliminate air bubbles and would be immediately read in continuous mode at an absorbance of 340 nm, for 10 time points in one-minute increments, for a total of 10 minutes. The slope from these 10 time points would be normalized to the positive and negative control wells. The duplicate % inhibition values would then be averaged and fit to a Hill equation for dose response according to the Levenberg-Marquardt algorithm with the Hill equation maximum set to 100 and the minimum set to 0.

The results would report the IC₅₀ of each compound. Unless otherwise specified, IC₅₀ values would be reported as the geometric mean of at least 2 assay runs on separate days. The compounds of the present invention would be shown to not be potent inhibitors of human GYS2. Unless otherwise specified, IC₅₀ values would be reported as the geometric mean of at least two assay runs on separate days. Each run would represent the average of a technical replicate, where each compound was assayed twice in the same plate.

### Example B-4

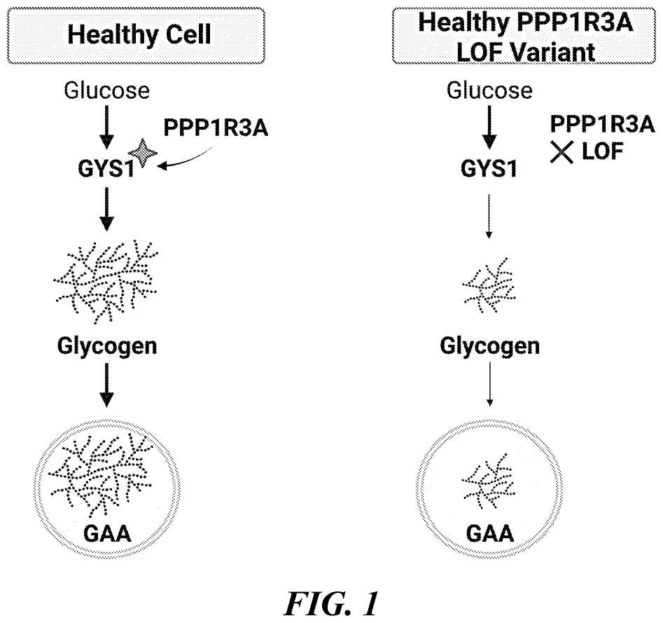

Pompe disease is a glycogen storage disease caused by mutations in the enzyme acid alpha-glucosidase resulting in pathological accumulation of glycogen. Glycogen can accumulate in virtually all tissues, but the primary pathology affects skeletal and cardiac muscle. Inhibiting the synthesis of muscle glycogen could reduce the pathologic build-up of glycogen by acting as a substrate reduction therapy. Savage et. al. identified a predicted protein truncating variant (PTV) in the PPP1R3A gene (a regulator of glycogen metabolism) in ˜0.5% of Europeans, which results in ˜65% reduction in muscle glycogen (Savage et. al., A Prevalent Variant in PPP1R3A Impairs Glycogen Synthesis and Reduces Muscle Glycogen Content in Humans and Mice. PLoS Medicine. 2008; herein incorporated by reference in its entirety). PPP1R3A functions as a key activator of muscle glycogen synthase 1 (GYS1) by dephosphorylating the enzyme and maximizing activity. FIG. 1 demonstrates the pathway in which PPP1R3A (loss of function) LoF leads to reduction in muscle glycogen.

Large biobanks enable investigation of the consequences of genetic variation on many health-related phenotypes. To assess the consequences of a predicted 65% loss of muscle glycogen, association study was performed in the UK Biobank comparing phenotypes between PPP1R3A PTV carriers and non-carriers. Genetic association studies were performed using REGENIE (Mbatchou, J., Barnard, L., Backman, J. et al. Computationally efficient whole-genome regression for quantitative and binary traits. Nat Genet 53, 1097-1103, 2021), adjusted for age, sex, and the first 10 principal components of ancestry. Quantitative traits were normalized using an inverse rank normal transformation.

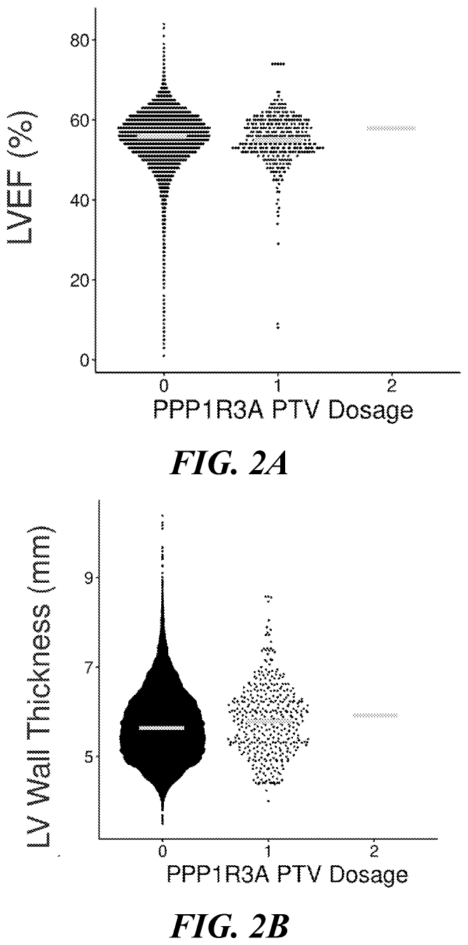

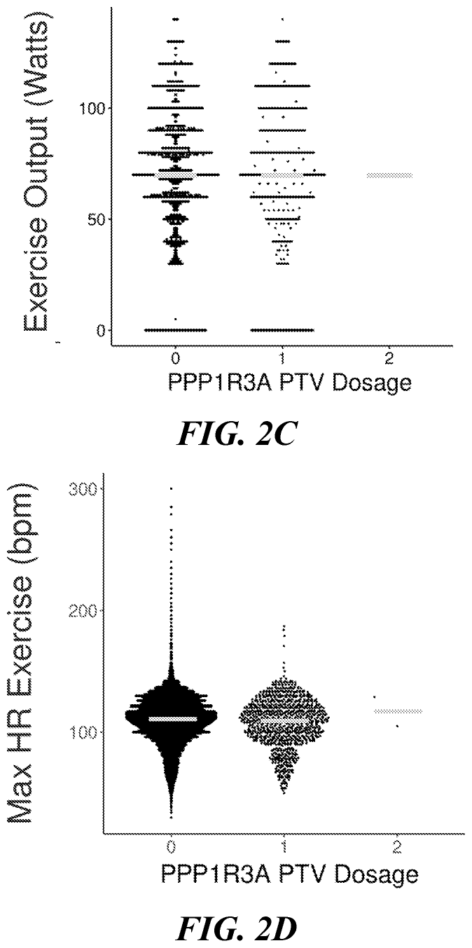

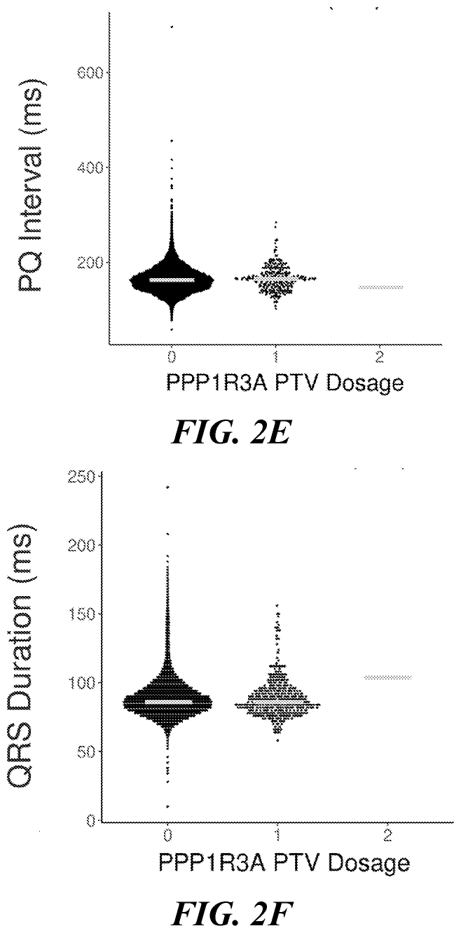

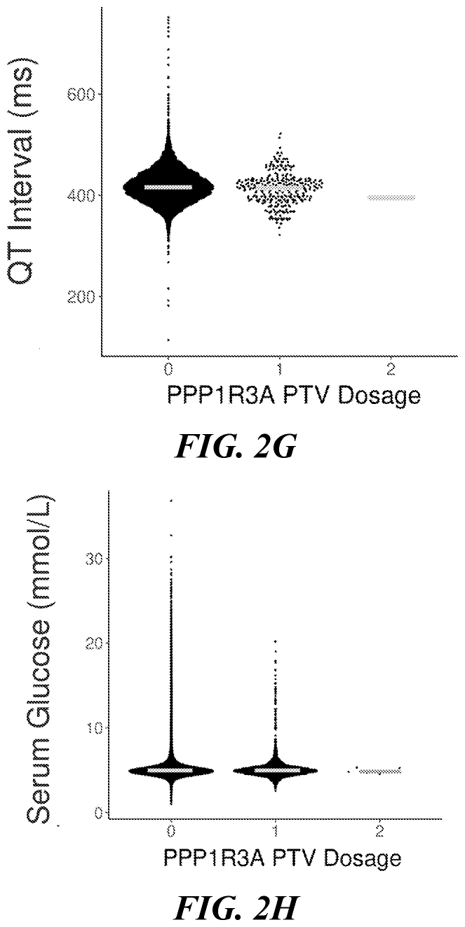

With regards to FIGS. 2A-2H, the association between PPP1R3A PTV and the quantitative phenotypes of left ventricular ejection (LVEF) (%) (FIG. 2A), left ventricle wall thickness (mm) (FIG. 2B), exercise output (watts) (FIG. 2C), max heart rate (HR) exercise (bpm) (FIG. 2D), PQ interval (ms) (FIG. 2E), QRS duration (ms) (FIG. 2F), QT interval (ms) (FIG. 2G), and serum glucose (mmol/L) (FIG. 2H), are depicted. Phenotype values are plotted by PPP1R3A dosage for UK Biobank participants. No association between PPP1R3A PTV and the quantitative phenotypes in the UK Biobank was identified.

Table 5 below lists the P-value and number of participants (N) for the results depicted in FIGS. 2A-H. No associations between PPP1R3A PTV and cardiac parameters, including left ventricular ejection fraction (p=0.871) and wall thickness (p=0.168) were identified. There was no evidence of changes in EKG cardiac conduction intervals nor in any muscle performance measurements (n=49,616), including maximum heart rate (p=0.444) and maximum workload during an exercise test (p=0.100). Further, no changes in serum glucose (p=0.71) or any other members of a panel of ˜170 serum metabolites were observed.

TABLE 5

| Phenotype | P-value | N |
|---|---|---|
| LVEF | 0.871 | 27,716 |
| LV Wall Thickness | 0.168 | 27,579 |
| Exercise Output | 0.100 | 49,616 |
| Max HR Exercise | 0.444 | 49,603 |
| QRS Duration | 0.527 | 29,507 |
| PQ Interval | 0.366 | 16,694 |
| QT Interval | 0.222 | 17,574 |
| Serum Glucose | 0.477 | 294,042 |

As shown in Table 6 below, no association between PPP1R3A PTV and key health outcomes was also observed. In addition to the phenotypes in Table 6, no phenome-wide significant associations between PPP1R3A PTV and rates of any ICD10 code with over 100 occurrences in UK Biobank was observed.

TABLE 6

| Disease | Effect (SE) | P-value | N Cases |
|---|---|---|---|
| Type 2 Diabetes | −0.094 (0.073) | 0.200 | 18,868 |
| Liver Cirrhosis | −0.041 (0.273) | 0.880 | 1,325 |
| Heart Failure | −0.061 (0.127) | 0.630 | 6,117 |

After performing an extensive Phenome-wide association study in UK Biobank, no significant associations between any key outcomes or phenotypes and loss of function of PPP1R3A were found. The results provided herein demonstrate that loss of function variants in the PPP1R3A gene are not associated with adverse health outcomes in a large biobank population. This suggests that partial reduction in muscle glycogen (˜65%) from birth is well tolerated and supports the potential safety of pharmacologic reduction of muscle glycogen.

All publications, patent applications, patents, and other references mentioned herein are expressly incorporated by reference in their entireties, to the same extent as if each were incorporated by reference individually.

It is to be understood that, while the disclosure has been described in conjunction with the above embodiments, the foregoing description and examples are intended to illustrate and not limit the scope of the disclosure. Other aspects, advantages and modifications within the scope of the disclosure will be apparent to those skilled in the art to which the disclosure pertains.

What is claimed is:

1. A compound of formula (I): 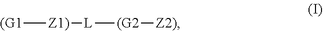
or a stereoisomer or tautomer thereof, or a pharmaceutically acceptable salt of any of the foregoing, wherein:
(i) G1 and/or G1-Z1, and (ii) G2 and/or G2-Z2 are each, independently, a GYS1 inhibiting moiety; and
L is a linker.

2. The compound of claim 1, or a stereoisomer or tautomer thereof, or a pharmaceutically acceptable salt of any of the foregoing, wherein L is selected from the group consisting of a bond or (C₁-C₅₀)alkyl, wherein
the (C₁-C₅₀)alkyl is optionally substituted with one or more deutero, halo, (C₁-C₆)alkyl, (C₁-C₆)haloalkyl, oxo (C═O), —O(C₁-C₆)alkyl, —O(C₁-C₆)haloalkyl, —O(C₁-C₆)cycloalkyl, —N(R^{x})(R^{y})—, C₃-C₁₅ carbocyclcyl, 3- to 15-membered heterocyclcyl, C₆-C₁₄ aryl, or 5- to 20-membered heteroaryl; and
one or more of the C atoms in the (C₁-C₅₀)alkyl is optionally replaced with one or more —C(R^{x})═C(R^{x})—, —C≡C—, —O—, —C(O)—, —C(O)O—, —OC(O)—, —OC(O)O—, —OC(O)N(R^{x})—, —N(R^{x})C(O)O—, —N(R^{x})C(O)N(R^{y})—, —C(O)N(R^{x})—, —N(R^{x})C(O)—, —N(R^{x})—, —S(O)ₙ—, —S(O)ₙO—, —OS(O)ₙ—, —OS(O)ₙO—, —OS(O)ₙN(R^{x})—, —N(R^{x})S(O)ₙO—, —N(R^{x})S(O)ₙN(R^{y})—, —S(O)ₙN(R^{x})—, —N(R^{k})S(O)ₙ—, C₃-C₁₅ carbocyclcyl, 3- to 15-membered heterocyclcyl, C₆-C₁₄ aryl, or 5- to 20-membered heteroaryl;
wherein,
each C₃-C₁₅ carbocyclcyl, 3- to 15-membered heterocyclcyl, C₆-C₁₄ aryl, or 5- to 20-membered heteroaryl is optionally substituted with one or more deutero, halo, (C₁-C₆)alkyl, (C₁-C₆)haloalkyl, oxo (C═O), —O(C₁-C₆)alkyl, —O(C₁-C₆)haloalkyl, —O(C₁-C₆)cycloalkyl, —N(R^{xx})(R^{yy}), —C(O)R^{z}, —C(O)OR^{z}, —OC(O)R^{z}, —OC(O)OR^{z}, —OC(O)N(R^{xx})(R^{yy}), —N(R^{xx})C(O)OR^{z}, —N(R^{xx})C(O)N((R^{xx})(R^{yy}), —C(O)N(R^{xx})(R^{yy}), —N(R^{xx})C(O)R^{z}, —N(R^{xx})(R^{yy}), —S(O)ₙR^{z}, —S(O)ₙOR^{z}, —OS(O)R^{z}, —OS(O)ₙOR^{z}, —OS(O)ₙN(R^{xx})(R^{yy}), —N(R^{xx})S(O)ₙOR^{z}, —N(R^{xx})S(O)ₙN(R^{x})(R^{y}), —S(O)ₙN(R^{xx})(R^{yy}), or —N(R^{xx})S(O)ₙR^{z};
each R^{x}, R^{xx}, R^{y}, R^{yy}, and R^{z} are independently H, (C₁-C₆)alkyl, (C₁-C₆)haloalkyl, or (C₁-C₆)cycloalkyl; and
each n is independently 0-2.

3. The compound of claim 2, wherein L is (C₁-C₅₀)alkyl optionally substituted with one or more deutero, halo, (C₁-C₆)alkyl, (C₁-C₆)haloalkyl, oxo (C═O), —O(C₁-C₆)alkyl, —O(C₁-C₆)haloalkyl, —O(C₁-C₆)cycloalkyl, —N(R^{x})(R^{y})—, C₃-C₁₅ carbocyclcyl, 3- to 15-membered heterocyclcyl, C₆-C₁₄ aryl, or 5- to 20-membered heteroaryl, wherein
one or more of the C atoms of the (C₁-C₅₀)alkyl is optionally replaced with one or more —C(R^{x})═C(R^{x})—, —C≡C—, —O—, —C(O)—, —N(R^{x})C(O)N(R^{y})—, —C(O)N(R^{x})—, —N(R^{x})C(O)—, —N(R^{x})—, —S(O)ₙ—, —N(R^{x})S(O)ₙN(R^{y})—, —S(O)ₙN(R^{x})—, —N(R^{x})S(O)ₙ—, C₃-C₁₅ carbocyclcyl, 3- to 15-membered heterocyclcyl, C₆-C₁₄ aryl, or 5- to 20-membered heteroaryl;
wherein
each C₃-C₁₅ carbocyclcyl, 3- to 15-membered heterocyclcyl, C₆-C₁₄ aryl, and 5- to 20-membered heteroaryl are optionally substituted with one or more deutero, halo, (C₁-C₆)alkyl, (C₁-C₆)haloalkyl, oxo (C═O), —O(C₁-C₆)alkyl, —O(C₁-C₆)haloalkyl, —O(C₁-C₆)cycloalkyl, —N(R^{xx})(R^{yy}), —C(O)R^{z}, —C(O)OR^{z}, —OC(O)R^{z}, —N(R^{xx})C(O)N((R^{xx})(R^{yy}), —C(O)N(R^{xx})(R^{yy}), —N(R^{xx})(R^{yy}), —S(O)ₙR^{z}, —N(R^{xx})S(O)ₙN(R^{xx})(R^{yy}), —S(O)ₙN(R^{xx})(R^{yy}), or —N(R^{xx})S(O)ₙR^{z};
each R^{x}, R^{xx}, R^{y}, R^{yy}, and R^{z} are independently H, (C₁-C₆)alkyl, (C₁-C₆)haloalkyl, or (C₁-C₆)cycloalkyl; and
each n is independently 0-2.

4. The compound of claim 3, wherein L is (C₁-C₂₀)alkyl, wherein
the (C₁-C₂₀)alkyl is optionally substituted with one or more deutero, halo, (C₁-C₆)alkyl, (C₁-C₆)haloalkyl, oxo (C═O), —O(C₁-C₆)alkyl, —O(C₁-C₆)haloalkyl, —O(C₁-C₆)cycloalkyl, —N(R^{x})(R^{y})—, C₃-C₁₅ carbocyclcyl, 3- to 15-membered heterocyclcyl, C₆-C₁₄ aryl, or 5- to 20-membered heteroaryl; and
one or more of the C atoms of the (C₁-C₂₀)alkyl is optionally replaced with one or more —C(R^{x})═C(R^{x})—, —C≡C—, —O—, —C(O)—, —N(R^{x})C(O)N(R^{y})—, —C(O)N(R^{x})—, —N(R^{x})C(O)—, —N(R^{x})—, —S(O)ₙ—, —N(R^{x})S(O)ₙN(R^{y})—, —S(O)ₙN(R^{x})—, —N(R^{x})S(O)ₙ—, C₃-C₁₅ carbocyclcyl, 3- to 15-membered heterocyclcyl, C₆-C₁₄ aryl, or 5- to 20-membered heteroaryl;
wherein
each C₃-C₁₅ carbocyclcyl, 3- to 15-membered heterocyclcyl, C₆-C₁₄ aryl, and 5- to 20-membered heteroaryl are optionally substituted with one or more deutero, halo, (C₁-C₆)alkyl, (C₁-C₆)haloalkyl, oxo (C═O), —O(C₁-C₆)alkyl, —O(C₁-C₆)haloalkyl, —O(C₁-C₆)cycloalkyl, —N(R^{xx})(R^{yy}), —C(O)R^{z}, —C(O)OR^{z}, —OC(O)R^{z}, —N(R^{xx})C(O)N((R^{xx})(R^{yy}), —C(O)N(R^{xx})(R^{yy}), —N(R^{xx})(R^{yy}), —S(O)ₙR^{z}, —N(R^{xx})S(O)ₙN(R^{xx})(R^{yy}), —S(O)ₙN(R^{xx})(R^{yy}), or —N(R^{xx})S(O)ₙR^{z};
each R^{x}, R^{xx}, R^{y}, R^{yy}, and R^{z} are independently H, (C₁-C₆)alkyl, (C₁-C₆)haloalkyl, or (C₁-C₆)cycloalkyl; and
each n is independently 0-2.

5. The compound of claim 4, wherein
the (C₁-C₂₀)alkyl is optionally substituted with one or more deutero, halo, (C₁-C₆)alkyl, (C₁-C₆)haloalkyl, oxo (C═O), —O(C₁-C₆)alkyl, —O(C₁-C₆)haloalkyl, —O(C₁-C₆)cycloalkyl, —N(R^{x})(R^{y})—, C₃-C₈ cycloalkyl, 3- to 8-membered heterocyclcyl, C₆-C₁₀ aryl, or 5- to 10-membered heteroaryl; and
one or more of the C atoms of the (C₁-C₂₀)alkyl is optionally replaced with one or more —C(R^{x})═C(R^{y})—, —C≡C—, —O—, —C(O)—, —N(R^{x})C(O)N(R^{y})—, —C(O)N(R^{x})—, —N(R^{x})C(O)—, —N(R^{x})—, —S(O)ₙ—, —N(R^{x})S(O)ₙN(R^{y})—, —S(O)ₙN(R^{x})—, —N(R^{x})S(O)ₙ—, C₃-C₈ cycloalkyl, 3- to 8-membered heterocyclcyl, C₆-C₁₀ aryl, or 5- to 10-membered heteroaryl;
wherein
each C₃-C₈ cycloalkyl, 3- to 8-membered heterocyclcyl, C₆-C₁₀ aryl, and 5- to 10-membered heteroaryl are optionally substituted with one or more deutero, halo, (C₁-C₆)alkyl, (C₁-C₆)haloalkyl, oxo (C═O), —O(C₁-C₆)alkyl, —O(C₁-C₆)haloalkyl, —O(C₁-C₆)cycloalkyl, —N(R^{xx})(R^{yy}), —C(O)R^{z}, —C(O)OR^{z}, —OC(O)R^{z}, —N(R^{xx})C(O)N((R^{xx})(R^{yy}), —C(O)N(R^{xx})(R^{yy}), —N(R^{xx})(R^{yy}), —S(O)ₙR^{z}, —N(R^{xx})S(O)ₙN(R^{xx})(R^{yy}), —S(O)ₙN(R^{xx})(R^{yy}), or —N(R^{xx})S(O)ₙR^{z};
each R^{x}, R^{xx}, R^{y}, R^{yy}, and R^{z} are independently H, (C₁-C₆)alkyl, (C₁-C₆)haloalkyl, or (C₁-C₆)cycloalkyl; and
each n is independently 0-2.

6. The compound of claim 5, wherein
the (C₁-C₂₀)alkyl is optionally substituted with one or more deutero, halo, (C₁-C₆)alkyl, (C₁-C₆)haloalkyl, oxo (C═O), —O(C₁-C₆)alkyl, —O(C₁-C₆)haloalkyl, —O(C₁-C₆)cycloalkyl, —N(R^{x})(R^{y})—, C₃-C₈ cycloalkyl, 3- to 8-membered heterocyclcyl, C₆-C₁₀ aryl, or 5- to 10-membered heteroaryl; and
one or more of the C atoms of the (C₁-C₂₀)alkyl is optionally replaced with one or more —C(R^{x})═C(R^{x})—, —C≡C—, —O—, —C(O)—, —N(R^{x})C(O)N(R^{y})—, —C(O)N(R^{x})—, —N(R^{x})C(O)—, —N(R^{x})—, —S(O)ₙ—, —N(R^{x})S(O)ₙN(R^{y})—, —S(O)ₙN(R^{x})—, —N(R^{x})S(O)ₙ—, C₃-C₈ cycloalkyl, 3- to 8-membered heterocyclcyl, C₆-C₁₀ aryl, or 5- to 10-membered heteroaryl;
wherein
each R^{x} and R^{y} are independently H, (C₁-C₆)alkyl, (C₁-C₆)haloalkyl, or (C₁-C₆)cycloalkyl; and
each n is independently 0-2.

7. The compound of claim 6, wherein
the (C₁-C₂₀)alkyl is optionally substituted with one or more deutero, halo, (C₁-C₆)alkyl, (C₁-C₆)haloalkyl, oxo (C═O), —O(C₁-C₆)alkyl, —O(C₁-C₆)haloalkyl, —O(C₁-C₆)cycloalkyl, or —N(R^{x})(R^{y})—; and
one or more of the C atoms of the (C₁-C₂₀)alkyl is optionally replaced with one or more —O—, —C(O)—, —N(R^{x})C(O)N(R^{y})—, —C(O)N(R^{x})—, —N(R^{x})C(O)—, —N(R^{x})—, —S(O)ₙ—, —N(R^{x})S(O)ₙN(R^{y})—, —S(O)ₙN(R^{x})—, —N(R^{x})S(O)ₙ—, C₃-C₈ cycloalkyl, 3- to 8-membered heterocyclcyl, C₆-C₁₀ aryl, or 5- to 10-membered heteroaryl;
wherein
each R^{x} and R^{y} are independently H, (C₁-C₆)alkyl, (C₁-C₆)haloalkyl, or (C₁-C₆)cycloalkyl; and
each n is independently 0-2.

8. The compound of claim 7, wherein the (C₁-C₂₀)alkyl is optionally substituted with one or more deutero, halo, (C₁-C₄)alkyl, (C₁-C₄)haloalkyl, oxo (C═O), —O(C₁-C₄)alkyl, —O(C₁-C₄)haloalkyl, or —O(C₁-C₄)cycloalkyl; and
one or more of the C atoms of the (C₁-C₂₀)alkyl is optionally replaced with one or more —O—, —C(O)—, —N(R^{x})C(O)N(R^{y})—, —C(O)N(R^{x})—, —N(R^{x})C(O)—, —N(R^{x})—, 3- to 8-membered heterocyclcyl, C₆-C₁₀ aryl, or 5- to 10-membered heteroaryl;
wherein
each R^{x} and R^{y} are independently H, (C₁-C₃)alkyl, (C₁-C₃)haloalkyl, or (C₁-C₃)cycloalkyl; and
each n is independently 0-2.

9. The compound of claim 8, wherein one or more C atoms of the (C₁-C₂₀)alkyl is optionally replaced with one or more —O—, —C(O)—, —N(R^{x})C(O)N(R^{y})—, —C(O)N(R^{x})—, —N(R^{x})C(O)—, or —N(R^{x})—; wherein
each R^{x} and R^{y} are independently H, (C₁-C₃)alkyl, (C₁-C₃)haloalkyl, or (C₁-C₃)cycloalkyl; and
each n is independently 0-2.

10. The compound of claim 9, wherein one or more of the C atoms of the (C₁-C₂₀)alkyl is replaced with one or more —O—.

11. The compound of claim 10, wherein L is selected from the group consisting of -(—O—CH₂CH₂)ₘ—O—, wherein m is 1-12.

12. The compound of claim 1 or claim 2, or a stereoisomer or tautomer thereof, or a pharmaceutically acceptable salt of any of the foregoing, wherein L comprises an optionally substituted alkylene, optionally substituted heteroalkylene, optionally substituted alkenylene, optionally substituted heteroalkenylene, optionally substituted alkynylene, optionally substituted heteroalkynylene, optionally substituted carbocyclylene, optionally substituted heterocyclylene, optionally substituted arylene, optionally substituted heteroarylene, or any combination thereof.

13. The compound of any one of claims 1-2, or 12, or a stereoisomer or tautomer thereof, or a pharmaceutically acceptable salt of any of the foregoing, wherein L is selected from the group consisting of ethylene glycol, polyethylene glycol (PEG), and PEG derivatives.

14. The compound of any one of claims 1-2, 12, or 13, or a stereoisomer or tautomer thereof, or a pharmaceutically acceptable salt of any of the foregoing, wherein L comprises a polymer.

15. The compound of any one of claims 1-2, or 12-14, or a stereoisomer or tautomer thereof, or a pharmaceutically acceptable salt of any of the foregoing, wherein L is polyethylene glycol.

16. The compound of claim 15, or a stereoisomer or tautomer thereof, or a pharmaceutically acceptable salt of any of the foregoing, wherein the polyethylene glycol comprises from about 2 to about 20 ethylene glycol units.

17. The compound of any one of claims 1-16, or a stereoisomer or tautomer thereof, or a pharmaceutically acceptable salt of any of the foregoing, wherein G1 and G2 are identical and/or G1-Z1 and G2-Z2 are identical.

18. The compound of any one of claims 1-16, or a stereoisomer or tautomer thereof, or a pharmaceutically acceptable salt of any of the foregoing, wherein G1 and G2 are different and/or G1-Z1 and G2-Z2 are different.

19. The compound of any one of claims 1-16 or claim 18, or a stereoisomer or tautomer thereof, or a pharmaceutically acceptable salt of any of the foregoing, wherein one of (i) G1 or G1-Z1, and (ii) G2 or G2-Z2, has enhanced GYS1 inhibition compared to the other of (i) G1 or G1-Z1, and (ii) G2 or G2-Z2.

20. The compound of any one of claims 1-19, or a stereoisomer or tautomer thereof, or a pharmaceutically acceptable salt of any of the foregoing, wherein the compound of formula (I) has enhanced GYS1 inhibition compared to G1, G1-Z1, G2, and/or G2-Z2.

21. The compound of any one of claims 1-20, or a stereoisomer or tautomer thereof, or a pharmaceutically acceptable salt of any of the foregoing, wherein (G1-Z1) and (G2-Z2) are each, independently, of formula (I-1) or (I-2): 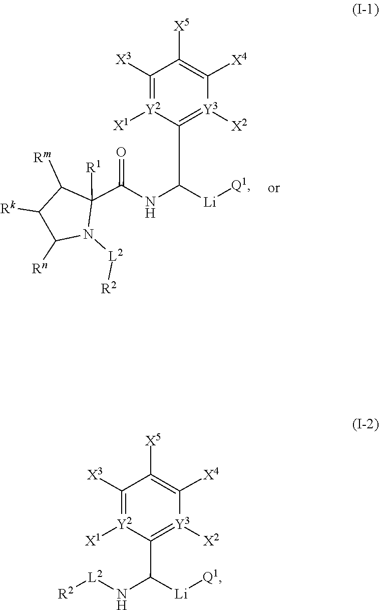
or a stereoisomer or tautomer thereof, or a pharmaceutically acceptable salt of any of the foregoing, wherein:
Y² and Y³ are each C, or
one of Y² and Y³ is N and the other of Y² and Y³ is C;
X¹ and X² are each independently H, C₁₋₆alkyl, or C₁₋₆alkoxy;
X³ and X⁴ are each independently H, halo, C₁₋₆alkyl, C₁₋₆alkoxy, or 5-20 membered heteroaryl, wherein the C₁₋₆alkyl of X³ and X⁴ is optionally substituted with one of more halo;
X⁵ is H, C₁₋₆alkyl, C₁₋₆alkoxy, or C₃₋₁₀cycloalkyl;
either
(1) L¹ is absent; and
Q¹ is selected from (i) to (iv):
(i) phenyl, wherein the phenyl of Q¹ is substituted with one or more halo, C₁₋₆alkyl, C₂₋₆alkenyl, —NH₂, —NH—C(O)—(C₁₋₆alkyl), —NH—C(O)-(3-15 membered heterocyclyl), C₃₋₁₀cycloalkyl, or 5-20 membered heteroaryl, wherein
the C₁₋₆alkyl is optionally substituted with one or more halo, —NH—C(O)—NH(C₁₋₆alkyl), —NH—C(O)—C₁₋₆alkyl, or —NH—C(O)—C₁₋₆alkoxy,
the C₃₋₁₀cycloalkyl is optionally substituted with one or more halo or C₁₋₆alkyl, and
the 5-20 membered heteroaryl is optionally substituted with one or more C₁₋₆alkyl,
(ii) 3-15 membered heterocyclyl, wherein the 3-15 membered heterocyclyl of Q¹ is optionally substituted with one or more oxo, or C₁₋₆alkyl,
(iii) 5-20 membered heteroaryl, wherein the 5-20 membered heteroaryl of Q¹ is optionally substituted with one or more halo, C₁₋₆alkyl, C₂₋₆alkenyl, C₁₋₆alkoxy, —NH₂, or C₃₋₁₀cycloalkyl, wherein,
the C₁₋₆alkyl is optionally substituted with one or more halo, and
the C₃₋₁₀cycloalkyl is optionally substituted with one or more halo or C₁₋₆alkyl, and
(iv) C₃₋₁₀cycloalkyl;
or
(2) L¹ is —CH₂—; and
Q¹ is C₃₋₁₀cycloalkyl;
L² is —C(O)— or —S(O)₂—
R¹ is H or C₁₋₆alkyl;
R^{k} is H, halo, —OH, —NH₂, or —NH—C(O)C₁₋₆alkyl;
R^{m} is H, —OH, or C₁₋₆alkyl;
Rⁿ is H, C₁₋₆alkyl, or C₃₋₁₀cycloalkyl or Rⁿ taken together with the carbon atom to which it is attached forms C₃₋₅ cycloalkyl;
or R^{k} is taken together with either R^{m} or Rⁿ, and the atoms to which they are attached, to form cyclopropyl; and
R² is selected from (i) to (vii):
(i) C₁₋₆alkyl, wherein the C₁₋₆alkyl of R² is optionally substituted with one or more R^{a}, wherein R^{a} is:
(a) —OH,
(b) cyano,
(c) C₂₋₆alkynyl,
(d) C₆₋₂₀aryl, wherein the C₆₋₂₀aryl of R^{a} is optionally substituted with one or more halo, cyano, C₁₋₆alkoxy, or —NH—C(O)—C₁₋₆alkyl,
(e) 3-15 membered heterocyclyl, wherein the 3-15 membered heterocyclyl of R^{a} is optionally substituted with one or more R^{c}, wherein
R^{c} is halo, oxo, C₁₋₆alkyl, C₁₋₆alkoxy, —C(O)—C₁₋₆alkyl, or —C(O)—C₁₋₆alkoxy, wherein
the C₁₋₆alkyl of R^{c} is optionally substituted with one or more halo or C₂₋₆alkynyl, and
the —C(O)—C₁₋₆alkoxy of R^{c} is optionally substituted with one or more halo,
(f) —N(R^{c})(R^{d}), wherein R^{c} and R^{d} of —N(R^{c})(R^{d}) are, independently of each other, H, C₁₋₆alkyl, —C(O)—C₁₋₆alkyl, —C(O)—C₁₋₆alkoxy, —C(O)—NH₂, —C(O)—NH(C₁₋₆alkyl), —C(O)—N(C₁₋₆alkyl)₂, —C(O)-(3-15 membered heterocyclyl), —CH₂—C(O)—NH₂, 3-15 membered heterocyclyl, or 5-20 membered heteroaryl, wherein
the C₁₋₆alkyl of R^{c} or R^{d} is optionally substituted with one or more —C(O)—NH₂,
the —C(O)—C₁₋₆alkyl of R^{c} or R^{d} is optionally substituted with one or more halo,
the 3-15 membered heterocyclyl and the 5-20 membered heteroaryl of R^{c} or R^{d} are independently optionally substituted with one or more C₁₋₆alkyl,
the —C(O)-(3-15 membered heterocyclyl) of R^{c} or R^{d} is optionally substituted with one or more halo, —C(O)—C₁₋₆alkoxy, or C₁₋₆alkyl, wherein the C₁₋₆alkyl is optionally substituted with one or more halo, C₁₋₆alkoxy, or C₃₋₁₀cycloalkyl, and
the C₁₋₆alkyl of the —C(O)—N(C₁₋₆alkyl)₂ of R^{c} or R^{d} are, independently of each other, optionally substituted with one or more halo or C₆₋₂₀aryl,
(g) —O—R^{e}, wherein R^{e} is C₁₋₆alkyl, C₆₋₂₀aryl, —C(O)-(3-15 membered heterocyclyl), —C(O)—N—(C₁₋₆alkyl)₂, or 5-20 membered heteroaryl, wherein
the C₁₋₆alkyl of R^{e} is optionally substituted with one or more C₁₋₆alkoxy, wherein the C₁₋₆alkoxy is optionally substituted with one or more C₂₋₆alkynyl,
the C₆₋₂₀aryl of R^{e} is optionally substituted with one or more C₁₋₆alkyl, and
the —C(O)-(3-15 membered heterocyclyl) of R^{e} is optionally substituted with one or more C₁₋₆alkyl, C₁₋₆alkoxy, or —C(O)—C₁₋₆alkoxy, wherein the C₁₋₆alkyl is optionally substituted with one or more halo, C₁₋₆alkoxy, or C₃₋₁₀cycloalkyl,
(h) —C(O)—R^{e}, wherein R^{e} is —NH₂, —OH, or 3-15 membered heterocyclyl, or
(i) —S(O)₂—R^{f}, wherein R^{f} is C₁₋₆alkyl or 3-15 membered heterocyclyl,
(ii) C₃₋₁₀cycloalkyl, wherein the C₃₋₁₀cycloalkyl of R² is optionally substituted with one or more R^{q}, wherein R^{q} is 5-20 membered heteroaryl or C₆₋₂₀aryl, wherein the C₆₋₂₀aryl of R^{q} is optionally substituted with one or more C₁₋₆alkoxy,
(iii) 3-15 membered heterocyclyl, wherein the 3-15 membered heterocyclyl of R² is optionally substituted with one or more halo, oxo, C₁₋₆alkyl, —C(O)—C₁₋₆alkyl, or 5-20 membered heteroaryl,
(iv) 5-20 membered heteroaryl or —(C₁₋₄alkyl)(5-20 membered heteroaryl), wherein the C₁₋₄ alkyl is optionally substituted with one or more or more —OH, halo, —NH₂, —NH(C₁₋₆alkyl), —N(C₁₋₆alkyl)₂, and wherein the 5-20 membered heteroaryl of the 5-20 membered heteroaryl or —(C₁₋₄alkyl)(5-20 membered heteroaryl), is optionally substituted with one or more R^{s}, wherein
R^{s} is halo, C₁₋₆alkyl, C₁₋₆alkoxy, —NH₂, —NH(C₁₋₆alkyl), —N(C₁₋₆alkyl)₂, —NH—C(O)—C₁₋₆alkyl, C₆₋₂₀aryl, C₃₋₁₀cycloalkyl, 3-15 membered heterocyclyl, 5-20 membered heteroaryl, or —C(O)—C₁₋₆alkoxy, wherein
the C₁₋₆alkyl of R^{s} is optionally substituted with one or more halo, C₁₋₆alkoxy, —NH₂, —NH(C₁₋₆alkyl), —N(C₁₋₆alkyl)₂, —NH—C(O)C₁₋₆alkyl, or —NH—C(O)—C₁₋₆alkoxy, and
the 3-15-membered heterocyclyl of R^{s} is optionally substituted with one or more halo or —C(O)—C₁₋₆alkoxy,
(v) —N(R^{g})(R^{h}), wherein R^{g} and R^{h} are independently H or C₁₋₆alkyl,
(vi) —C(O)—R^{j}, wherein R^{j} is C₃₋₁₀cycloalkyl, —NH(C₁₋₆alkyl), —N(C₁₋₆alkyl)₂, or —NH(5-20 membered heteroaryl), and
(vii) C₆₋₂₀aryl, wherein the C₆₋₂₀aryl of R² is optionally substituted with one or more 5-20 membered heteroaryl or —O—R^{p}, wherein R^{p} is 3-15 membered heterocyclyl, wherein the 3-15 membered heterocyclyl of R^{p} is optionally substituted with one or more —C(O)—C₁₋₆alkyl; and
wherein (G1-Z1) and (Z2-G2) are each independently substituted with a group bound to L.

22. The compound of claim 21, wherein when R² is unsubstituted methyl, then either:
(1) Q¹ is 5-20 membered heteroaryl, wherein the 5-20 membered heteroaryl of Q¹ is optionally substituted with one or more halo, C₁₋₆alkyl, C₂₋₆alkenyl, C₁₋₆alkoxy, —NH₂, C₃₋₁₀cycloalkyl, or —OH, and wherein Q¹ is not unsubstituted pyridyl, or
(2) Q¹ is phenyl, wherein the phenyl of Q¹ is substituted with
(i) at least one C₃₋₆alkyl, wherein the at least one C₃₋₆alkyl is optionally substituted with one or more halo, or
(ii) at least one C₃₋₁₀cycloalkyl, wherein the at least one C₃₋₁₀cycloalkyl is optionally substituted with one or more halo or C₁₋₆alkyl, or
(iii) at least one 5-20 membered heteroaryl, wherein the at least one 5-20 membered heteroaryl is optionally substituted with one or more C₁₋₆alkyl.

23. The compound of claim 21 wherein the group bound to L comprises a carbon, oxygen or nitrogen atom.

24. The compound of claim 21 or claim 22, or a stereoisomer or tautomer thereof, or a pharmaceutically acceptable salt of any of the foregoing, wherein G1 and G2 are each, independently, the moiety of formula (I-1) represented by 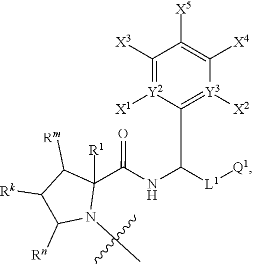
or the moiety of formula (I-1) or (I-2) represented by 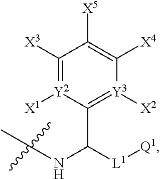
wherein R^{k}, R^{m}, Rⁿ, R¹, X¹, X², X³, X⁴, X⁵, Y², Y³, L¹, and Q¹ are as defined for a compound of formula (I-1), or (I-2), as defined in claim 21.

25. The compound of any one of claims 21-24, or a stereoisomer or tautomer thereof, or a pharmaceutically acceptable salt of any of the foregoing, wherein (Z1) and (Z2) are each, independently, 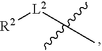
wherein L² and R² are as defined for a compound of formula (I-1), or (I-2), as defined in claim 21.

26. The compound of any one of claims 21-25, or a stereoisomer or tautomer thereof, or a pharmaceutically acceptable salt of any of the foregoing, wherein the moiety represented by 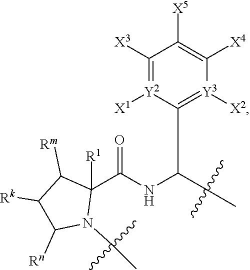
with carbon atoms bearing moieties 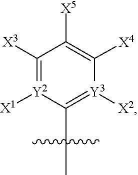
R^{k}, R^{m}, Rⁿ, and R¹, has a stereochemical configuration of the formula:

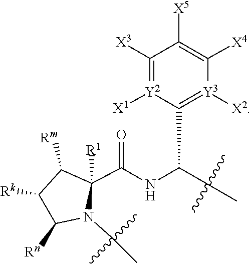

27. The compound of any one of claims 1-26, or a stereoisomer or tautomer thereof, or a pharmaceutically acceptable salt of any of the foregoing, wherein L is bound to:
(i) the Z1 moiety of (G1-Z1); and
(ii) the Z2 moiety of (G2-Z2).

28. The compound of any one of claims 1-26, or a stereoisomer or tautomer thereof, or a pharmaceutically acceptable salt of any of the foregoing, wherein L is bound to:
(i) the G1 moiety of (G1-Z1); and
(ii) the G2 moiety of (G2-Z2).

29. The compound of any one of claims 1-17, or 19-27, or a stereoisomer or tautomer thereof, or a pharmaceutically acceptable salt of any of the foregoing, wherein the compound is a compound of formula (II):

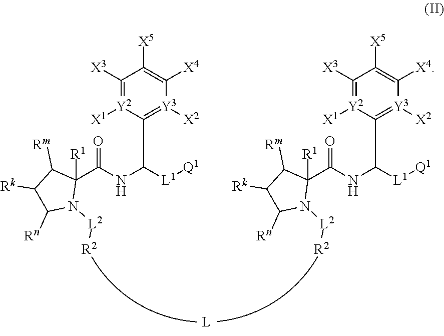

30. The compound of any one of claims 1-17, or 19-25, or 27, or a stereoisomer or tautomer thereof, or a pharmaceutically acceptable salt of any of the foregoing, wherein the compound is a compound of formula (III):

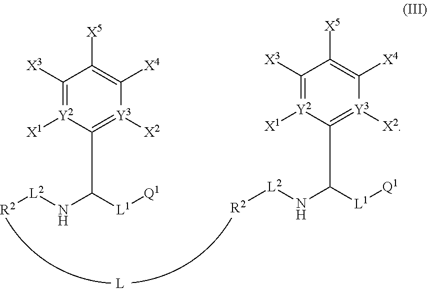

31. The compound of any one of claims 1-16, or 18-26 or a stereoisomer or tautomer thereof, or a pharmaceutically acceptable salt of any of the foregoing, wherein the compound is a compound of formula (IV):

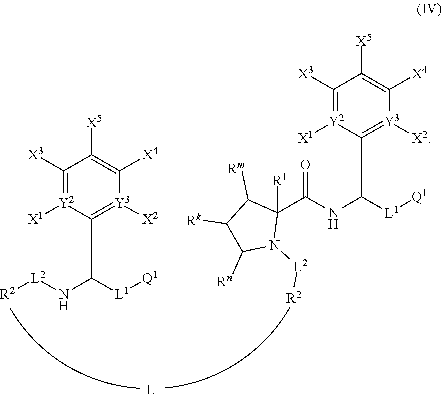

32. The compound of any one of claims 1-17, or 19-27, or a stereoisomer or tautomer thereof, or a pharmaceutically acceptable salt of any of the foregoing, wherein the compound is a compound of formula (V):

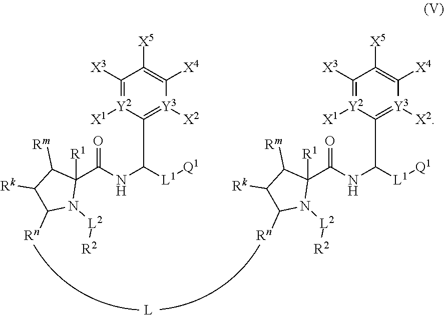

33. The compound of any one of claims 21, 29, 31, or 32, or a stereoisomer or tautomer thereof, or a pharmaceutically acceptable salt of any of the foregoing, wherein independently for each L, L¹, L², Y², Y³, R¹, R², R^{k}, R^{m}, Rⁿ, X¹, X², X³, X⁴, X⁵, and Q¹:
one of Y² and Y³ is N and the other of Y² and Y³ is C;
X¹ and X² are each independently H;
X³ and X⁴ are each independently H, or halo;
X⁵ is C₃₋₆cycloalkyl;
L¹ is absent;
Q¹ is phenyl;
L² is —C(O)—;
R¹ is H;
R^{k} is halo;
R^{m} is H;
Rⁿ is H;
R² is selected from (i) to (ii):
(i) C₁₋₃alkyl, wherein the C₁₋₃alkyl of R² is optionally substituted with one or more R^{a}, wherein R^{a} is 3-10 membered heterocyclyl, wherein the 3-10 membered heterocyclyl of R^{a} is optionally substituted with one or more R^{c}, wherein R^{c} is oxo, or C₁₋₃alkyl, or
(ii) 5-10 membered heteroaryl or —(C₁₋₃alkyl)(5-10 membered heteroaryl);
L is (C₁-C₂₀)alkyl, wherein
one or more of the C atoms in the (C₁-C₂₀)alkyl is optionally replaced with one or more —O—, —N(R^{x})—, or 3- to 10-membered heterocyclcyl;
wherein,
each 3- to 10-membered heterocyclcyl is optionally substituted with one or more (C₁-C₃)alkyl, or oxo (C═O); and
each R^{x} is independently H.

34. The compound of any one of claims 21, 30, or 31, or a stereoisomer or tautomer thereof, or a pharmaceutically acceptable salt of any of the foregoing, wherein independently for each L, L¹, L², Y², Y³, R², X¹, X², X³, X⁴, X⁵, and Q¹:
one of Y² and Y³ is N and the other of Y² and Y³ is C;
X¹ and X² are each independently H;
X³ and X⁴ are each independently H, or halo;
X⁵ is C₃₋₆cycloalkyl;
L¹ is absent;
Q¹ is phenyl;
L² is —C(O)—;
R² is selected from (i) to (ii):
(i) C₁₋₃alkyl, wherein the C₁₋₃alkyl of R² is optionally substituted with one or more R^{a}, wherein R^{a} is 3-10 membered heterocyclyl, wherein the 3-10 membered heterocyclyl of R^{a} is optionally substituted with one or more R^{c}, wherein R^{c} is oxo, or C₁₋₃alkyl, or
(ii) 5-10 membered heteroaryl or —(C₁₋₃alkyl)(5-10 membered heteroaryl);
L is (C₁-C₂₀)alkyl, wherein
one or more of the C atoms in the (C₁-C₂₀)alkyl is optionally replaced with one or more —O—, —N(R^{x})—, or 3- to 10-membered heterocyclcyl;
wherein,
each 3- to 10-membered heterocyclcyl is optionally substituted with one or more (C₁-C₃)alkyl, or oxo (C═O); and
each R^{x} is independently H.

35. The compound of claim 1, or a stereoisomer or tautomer thereof, or a pharmaceutically acceptable salt of any of the foregoing, wherein the compound is selected from the group consisting of: 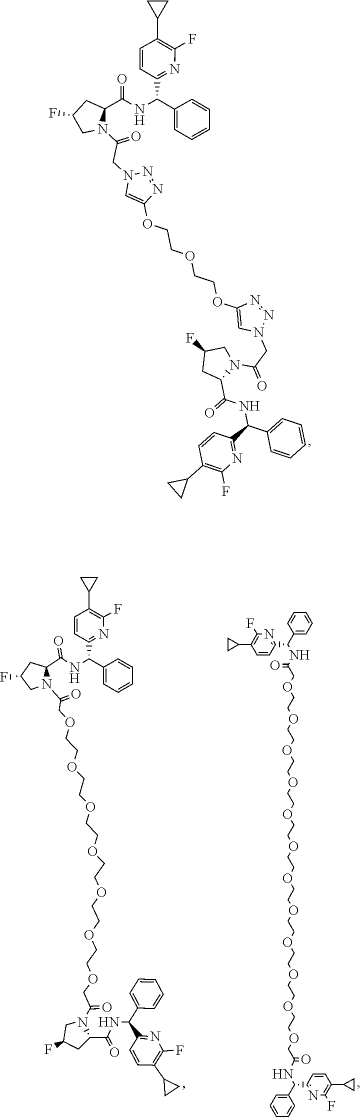 [SMILES: O=C(N[C@@H](c1ccccc1)c1ccc(C2CC2)c(F)n1)[C@@H]1C[C@@H](F)CN1C(=O)Cn1cc(OCCOCCOc2cn(CC(=O)N3C[C@H](F)C[C@H]3C(=O)N[C@@H](c3ccccc3)c3ccc(C4CC4)c(F)n3)nn2)nn1 ; O=C(N[C@@H](c1ccccc1)c1ccc(C2CC2)c(F)n1)[C@@H]1C[C@@H](F)CN1C(=O)COCCOCCOCCOCCOCCOCCOCC(=O)N1C[C@H](F)C[C@H]1C(=O)N[C@@H](c1ccccc1)c1ccc(C2CC2)c(F)n1 ; O=C(COCCOCCOCCOCCOCCOCCOCCOCCOCCOCCOCCOCC(=O)N[C@@H](c1ccccc1)c1ccc(C2CC2)c(F)n1)N[C@@H](c1ccccc1)c1ccc(C2CC2)c(F)n1] 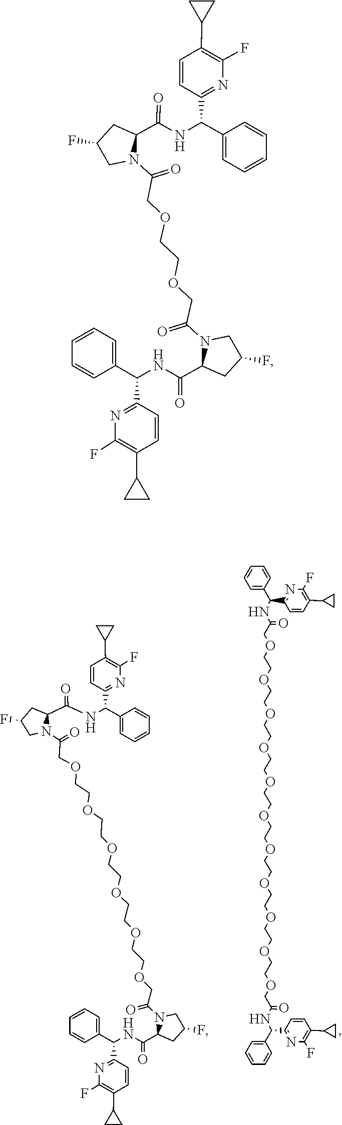 [SMILES: O=C(N[C@@H](c1ccccc1)c1ccc(C2CC2)c(F)n1)[C@@H]1C[C@@H](F)CN1C(=O)COCCOCC(=O)N1C[C@H](F)C[C@H]1C(=O)N[C@@H](c1ccccc1)c1ccc(C2CC2)c(F)n1 ; O=C(N[C@@H](c1ccccc1)c1ccc(C2CC2)c(F)n1)[C@@H]1C[C@@H](F)CN1C(=O)COCCOCCOCCOCCOCCOCC(=O)N1C[C@H](F)C[C@H]1C(=O)N[C@@H](c1ccccc1)c1ccc(C2CC2)c(F)n1 ; O=C(COCCOCCOCCOCCOCCOCCOCCOCCOCCOCCOCC(=O)N[C@@H](c1ccccc1)c1ccc(C2CC2)c(F)n1)N[C@@H](c1ccccc1)c1ccc(C2CC2)c(F)n1] 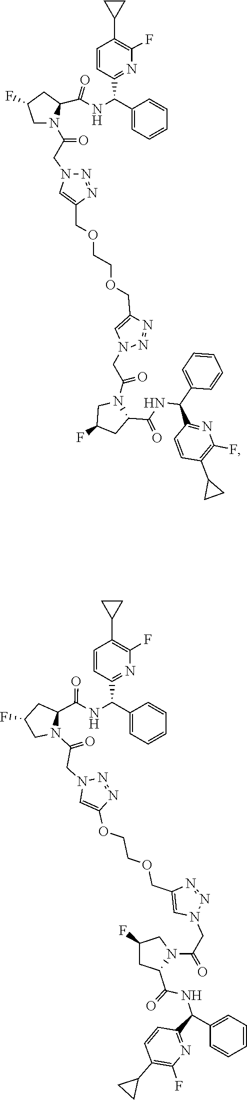 [SMILES: O=C(N[C@@H](c1ccccc1)c1ccc(C2CC2)c(F)n1)[C@@H]1C[C@@H](F)CN1C(=O)Cn1cc(COCCOCc2cn(CC(=O)N3C[C@H](F)C[C@H]3C(=O)N[C@@H](c3ccccc3)c3ccc(C4CC4)c(F)n3)nn2)nn1 ; O=C(N[C@@H](c1ccccc1)c1ccc(C2CC2)c(F)n1)[C@@H]1C[C@@H](F)CN1C(=O)Cn1cc(COCCOc2cn(CC(=O)N3C[C@H](F)C[C@H]3C(=O)N[C@@H](c3ccccc3)c3ccc(C4CC4)c(F)n3)nn2)nn1] 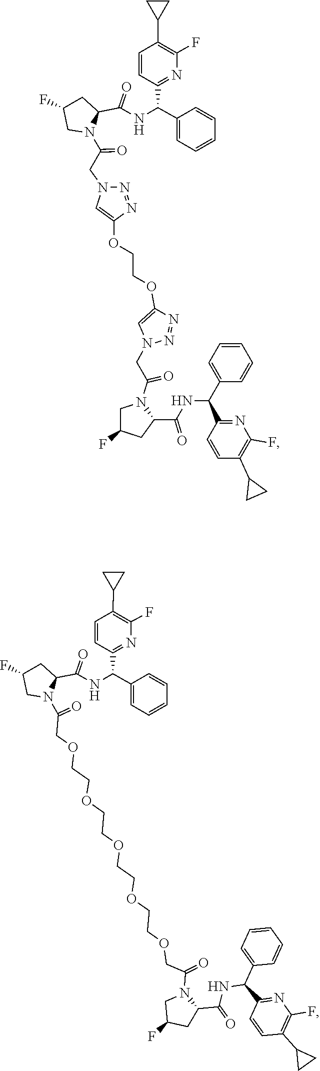 [SMILES: O=C(N[C@@H](c1ccccc1)c1ccc(C2CC2)c(F)n1)[C@@H]1C[C@@H](F)CN1C(=O)Cn1cc(OCCOc2cn(CC(=O)N3C[C@H](F)C[C@H]3C(=O)N[C@@H](c3ccccc3)c3ccc(C4CC4)c(F)n3)nn2)nn1 ; O=C(N[C@@H](c1ccccc1)c1ccc(C2CC2)c(F)n1)[C@@H]1C[C@@H](F)CN1C(=O)COCCOCCOCCOCCOCC(=O)N1C[C@H](F)C[C@H]1C(=O)N[C@@H](c1ccccc1)c1ccc(C2CC2)c(F)n1] 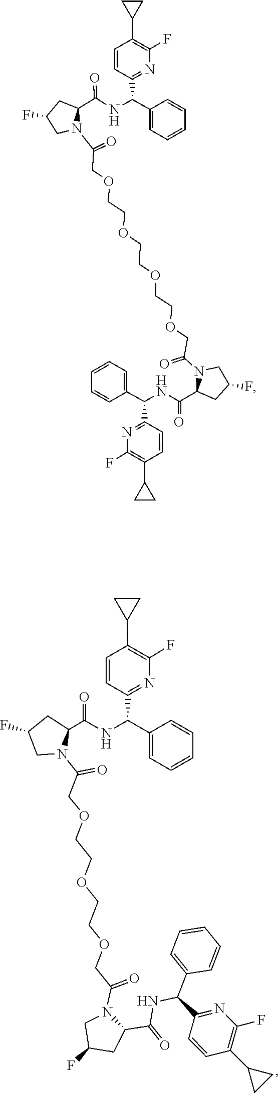 [SMILES: O=C(N[C@@H](c1ccccc1)c1ccc(C2CC2)c(F)n1)[C@@H]1C[C@@H](F)CN1C(=O)COCCOCCOCCOCC(=O)N1C[C@H](F)C[C@H]1C(=O)N[C@@H](c1ccccc1)c1ccc(C2CC2)c(F)n1 ; O=C(N[C@@H](c1ccccc1)c1ccc(C2CC2)c(F)n1)[C@@H]1C[C@@H](F)CN1C(=O)COCCOCCOCC(=O)N1C[C@H](F)C[C@H]1C(=O)N[C@@H](c1ccccc1)c1ccc(C2CC2)c(F)n1] 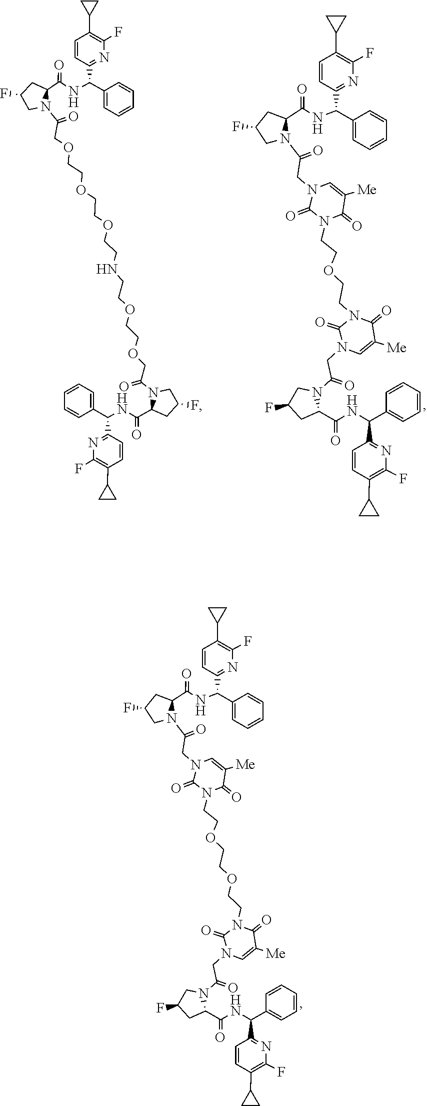 [SMILES: O=C(N[C@@H](c1ccccc1)c1ccc(C2CC2)c(F)n1)[C@@H]1C[C@@H](F)CN1C(=O)COCCOCCNCCOCCOCCOCC(=O)N1C[C@H](F)C[C@H]1C(=O)N[C@@H](c1ccccc1)c1ccc(C2CC2)c(F)n1 ; Cc1cn(CC(=O)N2C[C@H](F)C[C@H]2C(=O)N[C@@H](c2ccccc2)c2ccc(C3CC3)c(F)n2)c(=O)n(CCOCCn2c(=O)c(C)cn(CC(=O)N3C[C@H](F)C[C@H]3C(=O)N[C@@H](c3ccccc3)c3ccc(C4CC4)c(F)n3)c2=O)c1=O ; Cc1cn(CC(=O)N2C[C@H](F)C[C@H]2C(=O)N[C@@H](c2ccccc2)c2ccc(C3CC3)c(F)n2)c(=O)n(CCOCCOCCn2c(=O)c(C)cn(CC(=O)N3C[C@H](F)C[C@H]3C(=O)N[C@@H](c3ccccc3)c3ccc(C4CC4)c(F)n3)c2=O)c1=O] 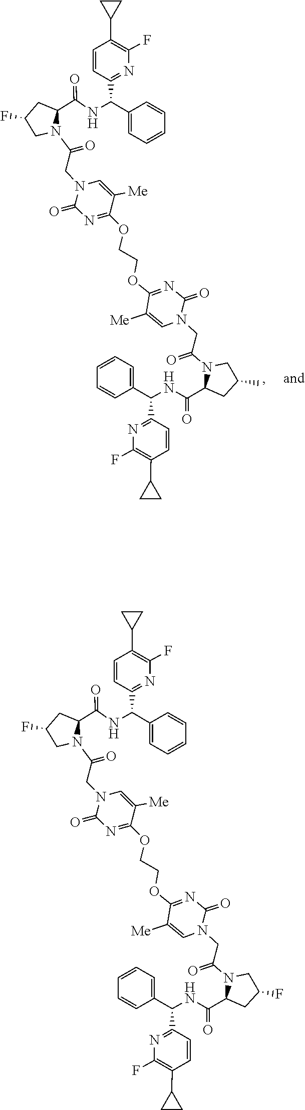 [SMILES: Cc1cn(CC(=O)N2C[C@H](C)C[C@H]2C(=O)N[C@@H](c2ccccc2)c2ccc(C3CC3)c(F)n2)c(=O)nc1OCCOc1nc(=O)n(CC(=O)N2C[C@H](F)C[C@H]2C(=O)N[C@@H](c2ccccc2)c2ccc(C3CC3)c(F)n2)cc1C ; Cc1cn(CC(=O)N2C[C@H](F)C[C@H]2C(=O)N[C@@H](c2ccccc2)c2ccc(C3CC3)c(F)n2)c(=O)nc1OCCOc1nc(=O)n(CC(=O)N2C[C@H](F)C[C@H]2C(=O)N[C@@H](c2ccccc2)c2ccc(C3CC3)c(F)n2)cc1C]
or a stereoisomer or tautomer thereof, or a pharmaceutically acceptable salt of any of the foregoing.

36. A process for preparing a compound of any one of claims 1-17, 19-27, or 29, or a stereoisomer or tautomer thereof, or a pharmaceutically acceptable salt of any of the foregoing, wherein the process comprises:
reacting a compound of formula (I-1B): 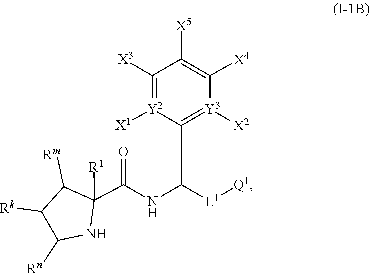
Y² and Y³ are each C, or
one of Y² and Y³ is N and the other of Y² and Y³ is C;
X¹ and X² are each independently H, C₁₋₆alkyl, or C₁₋₆alkoxy;
X³ and X⁴ are each independently H, halo, C₁₋₆alkyl, C₁₋₆alkoxy, or 5-20 membered heteroaryl, wherein the C₁₋₆alkyl of X³ and X⁴ is optionally substituted with one of more halo;
X⁵ is H, C₁₋₆alkyl, C₁₋₆alkoxy, or C₃₋₁₀cycloalkyl;
either
(1) L¹ is absent; and
Q¹ is selected from (i) to (iv):
(i) phenyl, wherein the phenyl of Q¹ is substituted with one or more halo, C₁₋₆alkyl, C₂₋₆alkenyl, —NH₂, —NH—C(O)—(C₁₋₆alkyl), —NH—C(O)-(3-15 membered heterocyclyl), C₃₋₁₀cycloalkyl, or 5-20 membered heteroaryl, wherein
the C₁₋₆alkyl is optionally substituted with one or more halo, —NH—C(O)—NH(C₁₋₆alkyl), —NH—C(O)—C₁₋₆alkyl, or —NH—C(O)—C₁₋₆alkoxy,
the C₃₋₁₀cycloalkyl is optionally substituted with one or more halo or C₁₋₆alkyl, and
the 5-20 membered heteroaryl is optionally substituted with one or more C₁₋₆alkyl,
(ii) 3-15 membered heterocyclyl, wherein the 3-15 membered heterocyclyl of Q¹ is optionally substituted with one or more oxo, or C₁₋₆alkyl,
(iii) 5-20 membered heteroaryl, wherein the 5-20 membered heteroaryl of Q¹ is optionally substituted with one or more halo, C₁₋₆alkyl, C₂₋₆alkenyl, C₁₋₆alkoxy, —NH₂, or C₃₋₁₀cycloalkyl, wherein,
the C₁₋₆alkyl is optionally substituted with one or more halo, and
the C₃₋₁₀cycloalkyl is optionally substituted with one or more halo or C₁₋₆alkyl, and
(iv) C₃₋₁₀cycloalkyl;
or
(2) L¹ is —CH₂—; and
Q¹ is C₃₋₁₀cycloalkyl;
R¹ is H or C₁₋₆alkyl;
R^{k} is H, halo, —OH, —NH₂, or —NH—C(O)C₁₋₆alkyl;
R^{m} is H, —OH, or C₁₋₆alkyl;
Rⁿ is H, C₁₋₆alkyl, or C₃₋₁₀cycloalkyl or Rⁿ taken together with the carbon atom to which it is attached forms C₃₋₅ cycloalkyl; and
or R^{k} is taken together with either R^{m} or Rⁿ, and the atoms to which they are attached, to form cyclopropyl;
with a compound of formula (L-1): 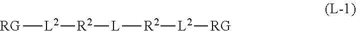
wherein
L² is —C(O)— or —S(O)₂—;
R² is selected from (i) to (vii):
(i) C₁₋₆alkyl, wherein the C₁₋₆alkyl of R² is optionally substituted with one or more R^{a} wherein R^{a} is:
(a) —OH,
(b) cyano,
(c) C₂₋₆alkynyl,
(d) C₆₋₂₀aryl, wherein the C₆₋₂₀aryl of R^{a} is optionally substituted with one or more halo, cyano, C₁₋₆alkoxy, or —NH—C(O)—C₁₋₆alkyl,
(e) 3-15 membered heterocyclyl, wherein the 3-15 membered heterocyclyl of R^{a} is optionally substituted with one or more R^{c}, wherein
R^{c} is halo, oxo, C₁₋₆alkyl, C₁₋₆alkoxy, —C(O)—C₁₋₆alkyl, or —C(O)—C₁₋₆alkoxy, wherein
the C₁₋₆alkyl of R^{c} is optionally substituted with one or more halo or C₂₋₆alkynyl, and
the —C(O)—C₁₋₆alkoxy of R^{c} is optionally substituted with one or more halo,
(f) —N(R^{c})(R^{d}), wherein R^{c} and R^{d} of —N(R^{c})(R^{d}) are, independently of each other, H, C₁₋₆alkyl, —C(O)—C₁₋₆alkyl, —C(O)—C₁₋₆alkoxy, —C(O)—NH₂, —C(O)—NH(C₁₋₆alkyl), —C(O)—N(C₁₋₆alkyl)₂, —C(O)-(3-15 membered heterocyclyl), —CH₂—C(O)—NH₂, 3-15 membered heterocyclyl, or 5-20 membered heteroaryl, wherein
the C₁₋₆alkyl of R^{c} or R^{d} is optionally substituted with one or more —C(O)—NH₂,
the —C(O)—C₁₋₆alkyl of R^{c} or R^{d} is optionally substituted with one or more halo,
the 3-15 membered heterocyclyl and the 5-20 membered heteroaryl of R^{c} or R^{d} are independently optionally substituted with one or more C₁₋₆alkyl,
the —C(O)-(3-15 membered heterocyclyl) of R^{c} or R^{d} is optionally substituted with one or more halo, —C(O)—C₁₋₆alkoxy, or C₁₋₆alkyl, wherein the C₁₋₆alkyl is optionally substituted with one or more halo, C₁₋₆alkoxy, or C₃₋₁₀cycloalkyl, and
the C₁₋₆alkyl of the —C(O)—N(C₁₋₆alkyl)₂ of R^{c} or R^{d} are, independently of each other, optionally substituted with one or more halo or C₆₋₂₀aryl,
(g) —O—R^{e}, wherein R^{e} is C₁₋₆alkyl, C₆₋₂₀aryl, —C(O)-(3-15 membered heterocyclyl), —C(O)—N—(C₁₋₆alkyl)₂, or 5-20 membered heteroaryl, wherein
the C₁₋₆alkyl of R^{e} is optionally substituted with one or more C₁₋₆alkoxy, wherein the C₁₋₆alkoxy is optionally substituted with one or more C₂₋₆alkynyl,
the C₆₋₂₀aryl of R^{e} is optionally substituted with one or more C₁₋₆alkyl, and
the —C(O)-(3-15 membered heterocyclyl) of R^{e} is optionally substituted with one or more C₁₋₆alkyl, C₁₋₆alkoxy, or —C(O)—C₁₋₆alkoxy, wherein the C₁₋₆alkyl is optionally substituted with one or more halo, C₁₋₆alkoxy, or C₃₋₁₀cycloalkyl,
(h) —C(O)—R^{e}, wherein R^{e} is —NH₂, —OH, or 3-15 membered heterocyclyl, or
(i) —S(O)₂—R^{f}, wherein R^{f} is C₁₋₆alkyl or 3-15 membered heterocyclyl,
(ii) C₃₋₁₀cycloalkyl, wherein the C₃₋₁₀cycloalkyl of R² is optionally substituted with one or more R^{q}, wherein R^{q} is 5-20 membered heteroaryl or C₆₋₂₀aryl, wherein the C₆₋₂₀aryl of R^{q} is optionally substituted with one or more C₁₋₆alkoxy,
(iii) 3-15 membered heterocyclyl, wherein the 3-15 membered heterocyclyl of R² is optionally substituted with one or more halo, oxo, C₁₋₆alkyl, —C(O)—C₁₋₆alkyl, or 5-20 membered heteroaryl,
(iv) 5-20 membered heteroaryl or —(C₁₋₄alkyl)(5-20 membered heteroaryl), wherein the C₁₋₄ alkyl is optionally substituted with one or more or more —OH, halo, —NH₂, —NH(C₁₋₆alkyl), —N(C₁₋₆alkyl)₂, and wherein the 5-20 membered heteroaryl of the 5-20 membered heteroaryl or —(C₁₋₄alkyl)(5-20 membered heteroaryl), is optionally substituted with one or more R^{s}, wherein
R^{s} is halo, C₁₋₆alkyl, C₁₋₆alkoxy, —NH₂, —NH(C₁₋₆alkyl), —N(C₁₋₆alkyl)₂, —NH—C(O)—C₁₋₆alkyl, C₆₋₂₀aryl, C₃₋₁₀cycloalkyl, 3-15 membered heterocyclyl, 5-20 membered heteroaryl, or —C(O)—C₁₋₆alkoxy, wherein
the C₁₋₆alkyl of R^{s} is optionally substituted with one or more halo, C₁₋₆alkoxy, —NH₂, —NH(C₁₋₆alkyl), —N(C₁₋₆alkyl)₂, —NH—C(O)C₁₋₆alkyl, or —NH—C(O)—C₁₋₆alkoxy, and
the 3-15-membered heterocyclyl of R^{s} is optionally substituted with one or more halo or —C(O)—C₁₋₆alkoxy,
(v) —N(R^{g})(R^{h}), wherein R^{g} and R^{h} are independently H or C₁₋₆alkyl,
(vi) —C(O)—R^{j}, wherein R^{j} is C₃₋₁₀cycloalkyl, —NH(C₁₋₆alkyl), —N(C₁₋₆alkyl)₂, or —NH(5-20 membered heteroaryl), and
(vii) C₆₋₂₀aryl, wherein the C₆₋₂₀aryl of R² is optionally substituted with one or more 5-20 membered heteroaryl or —O—R^{p}, wherein R^{p} is 3-15 membered heterocyclyl, wherein the 3-15 membered heterocyclyl of R^{p} is optionally substituted with one or more —C(O)—C₁₋₆alkyl; and
each RG is independently a reactive group suitable for attaching L-1 to the compound of formula (I-1B);
to form a compound of any one of claims 1-17, 19-27, or 29.

37. A process for preparing a compound of any one of claims 1-17, or 19-25, 27, or 30, or a stereoisomer or tautomer thereof, or a pharmaceutically acceptable salt of any of the foregoing, wherein the process comprises:
reacting a compound of formula (I-2B): 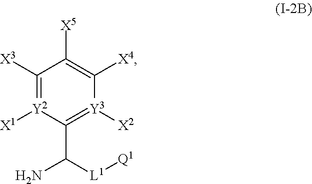
wherein,
Y² and Y³ are each C, or
one of Y² and Y³ is N and the other of Y² and Y³ is C;
X¹ and X² are each independently H, C₁₋₆alkyl, or C₁₋₆alkoxy;
X³ and X⁴ are each independently H, halo, C₁₋₆alkyl, C₁₋₆alkoxy, or 5-20 membered heteroaryl, wherein the C₁₋₆alkyl of X³ and X⁴ is optionally substituted with one of more halo;
X⁵ is H, C₁₋₆alkyl, C₁₋₆alkoxy, or C₃₋₁₀cycloalkyl;
either
(1) L¹ is absent; and
Q¹ is selected from (i) to (iv):
(i) phenyl, wherein the phenyl of Q¹ is substituted with one or more halo, C₁₋₆alkyl, C₂₋₆alkenyl, —NH₂, —NH—C(O)—(C₁₋₆alkyl), —NH—C(O)-(3-15 membered heterocyclyl), C₃₋₁₀cycloalkyl, or 5-20 membered heteroaryl, wherein
the C₁₋₆alkyl is optionally substituted with one or more halo, —NH—C(O)—NH(C₁₋₆alkyl), —NH—C(O)—C₁₋₆alkyl, or —NH—C(O)—C₁₋₆alkoxy,
the C₃₋₁₀cycloalkyl is optionally substituted with one or more halo or C₁₋₆alkyl, and
the 5-20 membered heteroaryl is optionally substituted with one or more C₁₋₆alkyl,
(ii) 3-15 membered heterocyclyl, wherein the 3-15 membered heterocyclyl of Q¹ is optionally substituted with one or more oxo, or C₁₋₆alkyl,
(iii) 5-20 membered heteroaryl, wherein the 5-20 membered heteroaryl of Q¹ is optionally substituted with one or more halo, C₁₋₆alkyl, C₂₋₆alkenyl, C₁₋₆alkoxy, —NH₂, or C₃₋₁₀cycloalkyl, wherein,
the C₁₋₆alkyl is optionally substituted with one or more halo, and
the C₃₋₁₀cycloalkyl is optionally substituted with one or more halo or C₁₋₆alkyl, and
(iv) C₃₋₁₀cycloalkyl;
or
(2) L¹ is —CH₂—; and
Q¹ is C₃₋₁₀cycloalkyl;
with a compound of formula (L-1): 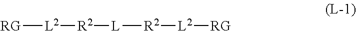
wherein
L² is —C(O)— or —S(O)₂—;
R² is selected from (i) to (vii):
(i) C₁₋₆alkyl, wherein the C₁₋₆alkyl of R² is optionally substituted with one or more R^{a} wherein R^{a} is:
(a) —OH,
(b) cyano,
(c) C₂₋₆alkynyl,
(d) C₆₋₂₀aryl, wherein the C₆₋₂₀aryl of R^{a} is optionally substituted with one or more halo, cyano, C₁₋₆alkoxy, or —NH—C(O)—C₁₋₆alkyl,
(e) 3-15 membered heterocyclyl, wherein the 3-15 membered heterocyclyl of R^{a} is optionally substituted with one or more R^{c}, wherein
R^{c} is halo, oxo, C₁₋₆alkyl, C₁₋₆alkoxy, —C(O)—C₁₋₆alkyl, or —C(O)—C₁₋₆alkoxy, wherein
the C₁₋₆alkyl of R^{c} is optionally substituted with one or more halo or C₂₋₆alkynyl, and
the —C(O)—C₁₋₆alkoxy of R^{c} is optionally substituted with one or more halo,
(f) —N(R^{c})(R^{d}), wherein R^{c} and R^{d} of —N(R^{c})(R^{d}) are, independently of each other, H, C₁₋₆alkyl, —C(O)—C₁₋₆alkyl, —C(O)—C₁₋₆alkoxy, —C(O)—NH₂, —C(O)—NH(C₁₋₆alkyl), —C(O)—N(C₁₋₆alkyl)₂, —C(O)-(3-15 membered heterocyclyl), —CH₂—C(O)—NH₂, 3-15 membered heterocyclyl, or 5-20 membered heteroaryl, wherein
the C₁₋₆alkyl of R^{c} or R^{d} is optionally substituted with one or more —C(O)—NH₂,
the —C(O)—C₁₋₆alkyl of R^{c} or R^{d} is optionally substituted with one or more halo,
the 3-15 membered heterocyclyl and the 5-20 membered heteroaryl of R^{c} or R^{d} are independently optionally substituted with one or more C₁₋₆alkyl, the —C(O)-(3-15 membered heterocyclyl) of R^{c} or R^{d} is optionally substituted with one or more halo, —C(O)—C₁₋₆alkoxy, or C₁₋₆alkyl, wherein the C₁₋₆alkyl is optionally substituted with one or more halo, C₁₋₆alkoxy, or C₃₋₁₀cycloalkyl, and
the C₁₋₆alkyl of the —C(O)—N(C₁₋₆alkyl)₂ of R^{c} or R^{d} are, independently of each other, optionally substituted with one or more halo or C₆₋₂₀aryl,
(g) —O—R^{e}, wherein R^{e} is C₁₋₆alkyl, C₆₋₂₀aryl, —C(O)-(3-15 membered heterocyclyl), —C(O)—N—(C₁₋₆alkyl)₂, or 5-20 membered heteroaryl, wherein
the C₁₋₆alkyl of R^{e} is optionally substituted with one or more C₁₋₆alkoxy, wherein the C₁₋₆alkoxy is optionally substituted with one or more C₂₋₆alkynyl,
the C₆₋₂₀aryl of R^{e} is optionally substituted with one or more C₁₋₆alkyl, and
the —C(O)-(3-15 membered heterocyclyl) of R^{e} is optionally substituted with one or more C₁₋₆alkyl, C₁₋₆alkoxy, or —C(O)—C₁₋₆alkoxy, wherein the C₁₋₆alkyl is optionally substituted with one or more halo, C₁₋₆alkoxy, or C₃₋₁₀cycloalkyl,
(h) —C(O)—R^{e}, wherein R^{e} is —NH₂, —OH, or 3-15 membered heterocyclyl, or
(i) —S(O)₂—R^{f}, wherein R^{f} is C₁₋₆alkyl or 3-15 membered heterocyclyl,
(ii) C₃₋₁₀cycloalkyl, wherein the C₃₋₁₀cycloalkyl of R² is optionally substituted with one or more R^{q}, wherein R^{q} is 5-20 membered heteroaryl or C₆₋₂₀aryl, wherein the C₆₋₂₀aryl of R^{q} is optionally substituted with one or more C₁₋₆alkoxy,
(iii) 3-15 membered heterocyclyl, wherein the 3-15 membered heterocyclyl of R² is optionally substituted with one or more halo, oxo, C₁₋₆alkyl, —C(O)—C₁₋₆alkyl, or 5-20 membered heteroaryl,
(iv) 5-20 membered heteroaryl or —(C₁₋₄alkyl)(5-20 membered heteroaryl), wherein the C₁₋₄ alkyl is optionally substituted with one or more or more —OH, halo, —NH₂, —NH(C₁₋₆alkyl), —N(C₁₋₆alkyl)₂, and wherein the 5-20 membered heteroaryl of the 5-20 membered heteroaryl or —(C₁₋₄alkyl)(5-20 membered heteroaryl), is optionally substituted with one or more R^{s}, wherein
R^{s} is halo, C₁₋₆alkyl, C₁₋₆alkoxy, —NH₂, —NH(C₁₋₆alkyl), —N(C₁₋₆alkyl)₂, —NH—C(O)—C₁₋₆alkyl, C₆₋₂₀aryl, C₃₋₁₀cycloalkyl, 3-15 membered heterocyclyl, 5-20 membered heteroaryl, or —C(O)—C₁₋₆alkoxy, wherein
the C₁₋₆alkyl of R^{s} is optionally substituted with one or more halo, C₁₋₆alkoxy, —NH₂, —NH(C₁₋₆alkyl), —N(C₁₋₆alkyl)₂, —NH—C(O)C₁₋₆alkyl, or —NH—C(O)—C₁₋₆alkoxy, and
the 3-15-membered heterocyclyl of R^{s} is optionally substituted with one or more halo or —C(O)—C₁₋₆alkoxy,
(v) —N(R^{g})(R^{h}), wherein R^{g} and R^{h} are independently H or C₁₋₆alkyl,
(vi) —C(O)—R^{j}, wherein R^{j} is C₃₋₁₀cycloalkyl, —NH(C₁₋₆alkyl), —N(C₁₋₆alkyl)₂, or —NH(5-20 membered heteroaryl), and
(vii) C₆₋₂₀aryl, wherein the C₆₋₂₀aryl of R² is optionally substituted with one or more 5-20 membered heteroaryl or —O—R^{p}, wherein R^{p} is 3-15 membered heterocyclyl, wherein the 3-15 membered heterocyclyl of R^{p} is optionally substituted with one or more —C(O)—C₁₋₆alkyl; and
each RG is independently a reactive group suitable for attaching L-1 to the compound of formula (I-1B); and
to form a compound of any one of claims 1-17, or 19-25, 27, or 30.

38. The process of claim 36, or claim 37, wherein RG is an —OH group.

39. A pharmaceutical composition comprising (i) a compound of any one of claims 1-35, or a stereoisomer or tautomer thereof, or a pharmaceutically acceptable salt of any of the foregoing, and (ii) one or more pharmaceutically acceptable excipients.

40. A method of treating a GYS1-mediated disease, disorder, or condition in an individual in need thereof, comprising administering to the individual an effective amount of a compound of any one of claims 1-33, or a stereoisomer or tautomer thereof, or a pharmaceutically acceptable salt of any of the foregoing, or the pharmaceutical composition of claim 39.

41. The method of claim 40, wherein the disease, disorder, or condition is a glycogen storage disorder (GSD).

42. The method of claim 40 or claim 41, wherein the disease, disorder, or condition is selected from the group consisting of Pompe disease, Cori disease (GSD III), adult polyglucosan body disease (APBD), and Lafora disease.

43. The method of any one of claims 40-42, wherein the disease, disorder, or condition is Pompe disease.

44. The method of claim 40, wherein the disease, disorder, or condition is cancer.

45. The method of claim 44, wherein the disease, disorder, or condition is selected from the group consisting of Ewing sarcoma (ES), clear cell renal cell carcinoma (ccRCC), glycogen rich clear cell carcinoma (GRCC) breast cancer, non-small-cell lung carcinoma (NSCLC), and acute myeloid leukemia (AML).

46. The method of claim 40, wherein the individual has a GAA mutation.

47. The method of claim 46, wherein the GAA mutation comprises a loss-of-function mutation.

48. A kit, comprising (i) a compound of any one of claims 1-35, or a stereoisomer or tautomer thereof, or a pharmaceutically acceptable salt of any of the foregoing, or the pharmaceutical composition of claim 39, and (ii) instructions for use in treating an GYS1-mediated disease, disorder, or condition in an individual in need thereof.

49. The kit of claim 48, wherein the disease, disorder, or condition comprises a glycogen storage disorder (GSD).

50. The kit of claim 48 or claim 49, wherein the disease, disorder, or condition is selected from the group consisting of Pompe disease, Cori disease (GSD III), adult polyglucosan body disease (APBD), and Lafora disease.

51. The kit of any one of claims 48-50, wherein the disease, disorder, or condition is Pompe disease.

52. The kit of claim 51, wherein the disease, disorder, or condition is cancer.

53. The kit of claim 48 or claim 52, wherein the disease, disorder, or condition is selected from the group consisting of Ewing sarcoma (ES), clear cell renal cell carcinoma (ccRCC), glycogen rich clear cell carcinoma (GRCC) breast cancer, non-small-cell lung carcinoma (NSCLC), and acute myeloid leukemia (AML).

54. The kit of claim 48, wherein the individual has a GAA mutation.

55. The kit of claim 54, wherein the GAA mutation comprises a loss-of-function mutation.

56. A method of modulating GYS1 in a cell, comprising exposing the cell to a composition comprising an effective amount of a compound of any one of claims 1-35, or a stereoisomer or tautomer thereof, or a pharmaceutically acceptable salt of any of the foregoing, or the pharmaceutical composition of claim 39.

57. A method of inhibiting GYS1 in a cell, comprising exposing the cell to a composition comprising an effective amount of a compound of any one of claims 1-35, or a stereoisomer or tautomer thereof, or a pharmaceutically acceptable salt of any of the foregoing, or the pharmaceutical composition of claim 39.

58. A method of reducing tissue glycogen stores in an individual in need thereof, comprising administering to the individual an effective amount of a compound of any one of claims 1-35, or a stereoisomer or tautomer thereof, or a pharmaceutically acceptable salt of any of the foregoing, or the pharmaceutical composition of claim 39.

59. A method of treating a GYS1-mediated disease, disorder, or condition in an individual in need thereof, comprising subjecting the individual to glycogen substrate reduction therapy, wherein the glycogen substrate reduction therapy comprises administering to the individual an effective amount of a compound of any one of claims 1-35, or a stereoisomer or tautomer thereof, or a pharmaceutically acceptable salt of any of the foregoing, or the pharmaceutical composition of claim 39.

60. The method of claim 59, comprising subjecting the individual to glycogen substrate reduction therapy in combination with enzyme replacement therapy.

61. The method of claim 60, wherein the enzyme replacement therapy is selected from the group consisting of alglucosidase alfa (human recombinant alpha-glucosidase (human GAA)) Myozyme and Lumizyme.

62. The method of any one of claims 59-61, wherein the disease, disorder, or condition is a glycogen storage disorder (GSD).

63. The method of any one of claims 59-62, wherein the disease, disorder, or condition is selected from the group consisting of Pompe disease, Cori disease (GSD III), adult polyglucosan body disease (APBD), and Lafora disease.

64. The method of any one of claims 59-63, wherein the disease, disorder, or condition is Pompe disease.

65. The method of any one of claims 59-61, wherein the disease, disorder, or condition is cancer.

66. The method of any one of claims 59-61, or 65, wherein the disease, disorder, or condition is selected from the group consisting of Ewing sarcoma (ES), clear cell renal cell carcinoma (ccRCC), glycogen rich clear cell carcinoma (GRCC) breast cancer, non-small-cell lung carcinoma (NSCLC), and acute myeloid leukemia (AML).

67. The method of any one of claims 59-61, wherein the individual has a GAA mutation.

68. The method of claim 67, wherein the GAA mutation comprises a loss-of-function mutation.

69. The method of any one of claims 56-58 wherein the compound is selective for GYS1 over GYS2.

70. The method of claim 69, wherein the compound is greater than 500 or 1,000 or 1,500 or 1,700-fold selective for GYS1 over GYS2.

71. The method of any one of claims 40-47 or 57-70, comprising reducing the level of glycogen in skeletal muscle.

72. A compound of any one of claims 1-33, or a stereoisomer or tautomer thereof, or a pharmaceutically acceptable salt of any of the foregoing, or the pharmaceutical composition of claim 39, for use in treating a GYS1-mediated disease, disorder, or condition in an individual in need thereof.

73. A compound of any one of claims 1-35, or a stereoisomer or tautomer thereof, or a pharmaceutically acceptable salt of any of the foregoing, or the pharmaceutical composition of claim 39, for use in modulating GYS1 in a cell.

74. A compound of any one of claims 1-35, or a stereoisomer or tautomer thereof, or a pharmaceutically acceptable salt of any of the foregoing, or the pharmaceutical composition of claim 39, for use in inhibiting GYS1 in a cell.

75. A compound of any one of claims 1-35, or a stereoisomer or tautomer thereof, or a pharmaceutically acceptable salt of any of the foregoing, or the pharmaceutical composition of claim 39, for use in reducing tissue glycogen stores in an individual in need thereof.

76. A compound of any one of claims 1-35, or a stereoisomer or tautomer thereof, or a pharmaceutically acceptable salt of any of the foregoing, or the pharmaceutical composition of claim 39, for use in a glycogen substrate reduction therapy for treating a GYS1-mediated disease, disorder, or condition in an individual in need thereof.

77. Use of a compound of any one of claims 1-33, or a stereoisomer or tautomer thereof, or a pharmaceutically acceptable salt of any of the foregoing, or the pharmaceutical composition of claim 39, in the manufacture of a medicament for use in treating a GYS1-mediated disease, disorder, or condition in an individual in need thereof.

78. Use of a compound of any one of claims 1-35, or a stereoisomer or tautomer thereof, or a pharmaceutically acceptable salt of any of the foregoing, or the pharmaceutical composition of claim 39, in the manufacture of a medicament for use in modulating GYS1 in a cell.

79. Use of a compound of any one of claims 1-35, or a stereoisomer or tautomer thereof, or a pharmaceutically acceptable salt of any of the foregoing, or the pharmaceutical composition of claim 39, in the manufacture of a medicament for use in inhibiting GYS1 in a cell.

80. Use of a compound of any one of claims 1-35, or a stereoisomer or tautomer thereof, or a pharmaceutically acceptable salt of any of the foregoing, or the pharmaceutical composition of claim 39, in the manufacture of a medicament for use in reducing tissue glycogen stores in an individual in need thereof.

81. Use of a compound of any one of claims 1-35, or a stereoisomer or tautomer thereof, or a pharmaceutically acceptable salt of any of the foregoing, or the pharmaceutical composition of claim 39, in the manufacture of a medicament for use in a glycogen substrate reduction therapy for treating a GYS1-mediated disease, disorder, or condition in an individual in need thereof.
